(19) United States
(12) Patent Application Publication
(10) Pub. No.: US 2026/0286374 A1
(43) Pub. Date: Sep. 24, 2026

(54) METHODS AND COMPOSITIONS FOR GENOME EDITING IN PLANTS

(71) Applicant: Napigen, Inc., Wilmington, DE (US)

(72) Inventors: Hajime Sakai, Newark, DE (US); Emil M. Orozco, Jr., Cochranville, PA (US); Sachie Kimura, Wilmington, DE (US); Elmer Heppard, Wilmington, DE (US); Kamal Bhattarai, Newark, DE (US); Mina Hamidi, Elkton, MD (US); Colleen McMichael, Wilmington, DE (US); Cheryl S. Caster, Landenberg, PA (US); Dawn Eigner, Newark, DE (US); Debra J. Wilkinson, Newark, DE (US)

(21) Appl. No.: 19/445,949

(22) Filed: Jan. 12, 2026

Related U.S. Application Data
(60) Provisional application No. 63/744,608, filed on Jan. 13, 2025.

(51) Int. Cl. C12N 15/82; C07K 14/415

(52) U.S. Cl. CPC C12N 15/8214; C07K 14/415

(57) ABSTRACT

Various aspects of the present disclosure relate to methods and compositions for modulating or editing the genome of plants, including nuclear, plastid, and mitochondrial genomes. The disclosed methods employ genome editing tools, such as site-directed nucleases. Provided herein, in some aspects, are compositions (e.g., a plant) comprising the altered genome.

### CROSS-REFERENCE

This application claims the benefit of U.S. Provisional Application No. 63/744,608, filed Jan. 13, 2025, which is hereby incorporated by reference in its entirety.

### STATEMENT AS TO FEDERALLY SPONSORED RESEARCH

This invention was made with government support under Agreement No. 58-5062-0-001 awarded by the United States Department of Agriculture (USDA) for a Cooperative Research and Development Agreement (CRADA), and under Grant Number 2024-70501-41924 awarded by the Small Business Innovation Research Program (SBIR) of the United States Department of Agriculture National Institute of Food and Agriculture (USDA-NIFA). The government has certain rights in the invention.

### SEQUENCE LISTING

The instant application contains a Sequence Listing which has been submitted electronically in XML format and is hereby incorporated by reference in its entirety. Said XML copy, created on Jun. 1, 2026, is named 51090-708.201.xml and is 281,693 bytes in size.

### BACKGROUND

Modification of mitochondrial and plastid genomes is of immense importance for basic and applied research. Transgenic plants with stably modified mitochondrial or plastid genomes can have new traits such as herbicide tolerance, insect resistance, and/or accumulation of valuable proteins such as pharmaceutical proteins and industrial enzymes.

### SUMMARY OF THE INVENTION

Provided herein is a method for replacing an endogenous target polynucleotide present in a genome of a mitochondrion or a plastid of a cell with an internal polynucleotide, the method comprising: (a) introducing into the mitochondrion or the plastid of the cell a replacement nucleic acid comprising the internal polynucleotide, wherein the internal polynucleotide comprises a sequence heterologous to the genome of the mitochondrion or the plastid; (b) selecting an altered cell comprising at least one altered mitochondrion genome or at least one altered plastid genome, wherein the at least one altered mitochondrion genome or the at least one altered plastid genome comprises the internal polynucleotide; (c) introducing into the altered cell a polynucleotide encoding a site-directed nuclease (SDN) or the SDN, wherein the SDN binds to a target sequence present in the endogenous target polynucleotide; and (d) selecting a further altered cell, wherein at least about 50% of mitochondrial genomes of a plurality of mitochondrial genomes or at least about 50% of plastid genomes of a plurality of plastid genomes in the further altered cell comprise the internal polynucleotide. In some embodiments, the SDN cleaves and degrades a mitochondrial DNA or a plastid DNA lacking the internal polynucleotide. In some embodiments, the replacement nucleic acid and the polynucleotide encoding the SDN are introduced concurrently or sequentially. In some embodiments, the replacement nucleic acid further comprises a 5′-homologous region (5′-HR) polynucleotide and a 3′-homologous region (3′-HR) polynucleotide. In some embodiments, the replacement nucleic acid comprises, in 5′ to 3′ direction, (i) the 5′-HR polynucleotide; (ii) the internal polynucleotide; and (iii) the 3′-HR polynucleotide. In some embodiments, the 5′-HR polynucleotide comprises a first sequence homologous to a genomic region adjacent to a first end of the endogenous target polynucleotide. In some embodiments, the 3′-HR polynucleotide comprises a second sequence homologous to a genomic region adjacent to a second end of the endogenous target polynucleotide. In some embodiments the SDN is a modified SDN. In some embodiments, the SDN comprises an organelle targeting amino acid sequence (OTS). In some embodiments, the endogenous target polynucleotide further comprises an additional target sequence for an additional SDN. In some embodiments, the target sequence or the additional target sequence or both are not present in the replacement nucleic acid. In some embodiments, the OTS is a mitochondrion targeting amino acid sequence (MTS), a plastid targeting amino acid sequence (PTS), or an amino acid sequence that targets the mitochondrion and the plastid. In some embodiments, the OTS is the MTS. In some embodiments, the MTS comprises or consists of an amino acid sequence that is at least about 80% identical to an amino acid sequence of SEQ ID NO: 42 or SEQ ID NO: 36 or SEQ ID NO: 129. In some embodiments, the OTS is the PTS. In some embodiments, the PTS comprises or consists of an amino acid sequence that is at least about 80% identical to an amino acid sequence of SEQ ID NO: 88. In some embodiments, the SDN is selected from the group consisting of: a polynucleotide guided nuclease, a transcription activator-like effector nuclease (TALEN), a zinc-finger nuclease, a meganuclease, a restriction enzyme, and any combination thereof. In some embodiments, the SDN is the polynucleotide guided nuclease. In some embodiments, the polynucleotide guided nuclease is selected from the group consisting of: a Cas9 protein, a Cas3 protein, a MAD2 protein, a MAD7 protein, a CRISPR nuclease, a nuclease domain of a Cas protein, a Cpf1 protein, modified versions thereof, a biologically active fragment thereof, and any combination thereof. In some embodiments, the internal polynucleotide encodes a guide ribonucleic acid (RNA), wherein the guide RNA directs the polynucleotide guided nuclease to cleave the target sequence. In some embodiments, the internal polynucleotide further encodes a polycistronic RNA, wherein the guide RNA is present on the polycistronic RNA. In some embodiments, the guide RNA is arrayed with multiple tRNA sequences for processing from the polycistronic RNA. In some embodiments, the SDN is the TALEN. In some embodiments, the TALEN comprises a first polypeptide and a second polypeptide, wherein each of the first polypeptide and the second polypeptide comprise a nuclease domain, wherein the first polypeptide and the second polypeptide are capable of forming a dimer. In some embodiments, the first polypeptide comprises a first OTS, and the second polypeptide comprises a second OTS. In some embodiments, the first OTS and the second OTS are the same. In some embodiments, the first OTS and the second OTS are different. In some embodiments, the nuclease domain comprises or consists of an amino acid sequence that is at least about 80% identical to an amino sequence of SEQ ID NO: 3, SEQ ID NO: 5, or SEQ ID NO: 60. In some embodiments, the nuclease domain comprises or consists of an amino acid sequence of SEQ ID NO: 3, SEQ ID NO: 5, or SEQ ID NO: 60. In some embodiments, the SDN comprises a single-chain TALEN. In some embodiments, the single-chain TALEN comprises two copies of a nuclease domain. In some embodiments, the two copies are capable of forming a dimer. In some embodiments, the single-chain TALEN comprises the OTS. In some embodiments, the single-chain TALEN binds to a target sequence in the endogenous target polynucleotide and cleaves the endogenous target polynucleotide. In some embodiments, the nuclease domain comprises or consists of an amino acid sequence that is at least about 80% identical to an amino sequence of SEQ ID NO: 3, SEQ ID NO: 5, or SEQ ID NO: 60. In some embodiments, the nuclease domain comprises or consists of an amino acid sequence of SEQ ID NO: 3, SEQ ID NO: 5, or SEQ ID NO: 60. In some embodiments, the replacement nucleic acid is introduced into the mitochondrion or the plastid by biolistic particle bombardment. In some embodiments, the method further comprises introducing into a nucleus of the cell a third polynucleotide encoding an additional selectable marker polypeptide, wherein the additional selectable marker polypeptide provides tolerance to an additional selective agent, and selecting a cell that grows in the presence of the additional selective agent. In some embodiments, the cell is grown simultaneously in a presence of the additional selective agent and in a presence of the herbicide, wherein the additional selective agent and the herbicide are each present at an effective concentration. In some embodiments, the cell is grown sequentially first in a presence of the additional selective agent and subsequently in a presence of the herbicide, or is grown sequentially first in a presence of the herbicide and subsequently in a presence of the additional selective agent, wherein the additional selective agent and the herbicide are each present at an effective concentration. In some embodiments, the additional selectable marker polypeptide is a polypeptide with hygromycin phosphotransferase (HPT) activity and the additional selective agent is hygromycin. In some embodiments, a polypeptide or polynucleotide disclosed herein can be introduced into a cell or organelle by a suitable method disclosed herein. In some embodiments, the polynucleotide encoding the SDN is introduced into the altered cell by microinjection, meristem transformation, electroporation, Agrobacterium-mediated transformation, viral based gene transfer, transfection, vacuum infiltration, biolistic particle bombardment or any combination thereof. In some embodiments, the polynucleotide encoding the SDN is introduced into the altered cell by the Agrobacterium-mediated transformation. In some embodiments, the polynucleotide encoding the SDN is stably integrated into a nuclear genome of the cell. In some embodiments, the cell is selected from the group consisting of a yeast cell, an algal cell, a plant cell, an insect cell, a non-human animal cell, and a mammalian tissue culture cell. In some embodiments, the cell is the plant cell. In some embodiments, the plant cell is selected from the group consisting of a wheat cell, a maize cell, a rice cell, a barley cell, a sorghum cell, a rye cell, a canola cell, a broccoli cell, a cauliflower cell, a cotton cell, and a soybean cell. In some embodiments, a plant is grown from the plant cell. In some embodiments, the internal polynucleotide encodes a polypeptide selected from the group consisting of a cytoplasmic male sterility (CMS) polypeptide, a polypeptide with an herbicide-resistant enzyme activity, a polypeptide with an antibiotic-resistance enzyme activity, a polypeptide with phosphite dehydrogenase activity, and any combination thereof. In some embodiments, the CMS polypeptide comprises a rice orf79 polypeptide, and wherein the plant cell is a rice cell. In some embodiments, the orf79 polypeptide comprises an amino acid sequence having at least about 80% sequence identity to SEQ ID NO: 8. In some embodiments, the orf79 polypeptide is encoded by a nucleic acid sequence having at least about 80% sequence identity to SEQ ID NO: 29. In some embodiments, the CMS polypeptide comprises a rice orf79 polypeptide, a rice WA352 polypeptide, or both. In some embodiments, the rice WA352 polypeptide comprises an amino acid sequence having at least about 80% sequence identity to SEQ ID NO: 128. In some embodiments, the plant cell is a rice cell. In some embodiments, the CMS polypeptide comprises a wheat orf256 polypeptide or a wheat orf279 polypeptide, and wherein the plant cell is a wheat cell. In some embodiments, the orf256 polypeptide comprises an amino acid sequence having at least about 80% sequence identity to SEQ ID NO: 9. In some embodiments, the orf279 polypeptide comprises an amino acid sequence having at least about 80% sequence identity to SEQ ID NO: 10. In some embodiments, the CMS polypeptide comprises a wheat orf256 polypeptide, a wheat orf279 polypeptide, or both. In some embodiments, the plant cell is a wheat cell. In some embodiments, the polypeptide with the herbicide-resistant enzyme activity is a plant-derived polypeptide. In some embodiments, the herbicide-resistant enzyme activity is selected from the group consisting of an herbicide-resistant acetolactate synthase (ALS) activity, an herbicide-resistant glutamine synthetase (GS) activity, an herbicide-resistant 5-enol-pyruvyl-shikimate-3-phosphate synthase (EPSPS) activity, and any combination thereof. In some embodiments, the antibiotic-resistance enzyme activity is at least one selected from the group consisting of an aminoglycoside adenylyltransferase (aadA) activity, an hygromycin phosphotransferase (hpt) activity, a neomycin phosphotransferase activity (nptII), and any combination thereof. In some embodiments, at least about 60% of mitochondrial genomes of the plurality of mitochondrial genomes or at least about 60% of plastid genomes of the plurality of plastid genomes in the further altered cell comprise the internal polynucleotide. In some embodiments, at least about 70% of mitochondrial genomes of the plurality of mitochondrial genomes or at least about 70% of plastid genomes of the plurality of plastid genomes in the further altered cell comprise the internal polynucleotide. In some embodiments, at least about 80% of mitochondrial genomes of the plurality of mitochondrial genomes or at least about 80% of plastid genomes of the plurality of plastid genomes in the further altered cell comprise the internal polynucleotide. In some embodiments, at least about 90% of mitochondrial genomes of the plurality of mitochondrial genomes or at least about 90% of plastid genomes of the plurality of plastid genomes in the further altered cell comprise the internal polynucleotide. In some embodiments, at least about 95% of mitochondrial genomes of the plurality of mitochondrial genomes or at least about 95% of plastid genomes of the plurality of plastid genomes in the further altered cell comprise the internal polynucleotide. In some embodiments, at least about 98% of mitochondrial genomes of the plurality of mitochondrial genomes or at least about 98% of plastid genomes of the plurality of plastid genomes in the further altered cell comprise the internal polynucleotide. In some embodiments, the plurality of mitochondrial genomes or the plurality of plastid genomes comprises the internal polynucleotide. Provided herein is a cell produced by the method described herein. Further provided herein is a plant produced by the method described herein.

Provided herein is a plant comprising a cell having an altered mitochondrion genome or an altered plastid genome, wherein the altered mitochondrion genome or the altered plastid genome comprises a heterologous polynucleotide comprising an internal polynucleotide in place of an endogenous polynucleotide, wherein the plant comprises a polynucleotide encoding a site-directed nuclease (SDN), wherein the SDN binds to a target sequence present in the endogenous polynucleotide, and wherein at least about 50% of mitochondrial genomes of a plurality of mitochondrial genomes or at least about 50% of the plastid genomes of a plurality of plastid genomes in the cell comprise the internal polynucleotide. In some embodiments, the heterologous polynucleotide encodes a polypeptide selected from the group consisting of: a cytoplasmic male sterility (CMS) polypeptide, a polypeptide with an herbicide-resistant enzyme activity, a polypeptide with an antibiotic-resistance enzyme activity, a polypeptide with phosphite dehydrogenase activity, and any combination thereof. In some embodiments, the herbicide-resistant enzyme activity is selected from the group consisting of an herbicide-resistant acetolactate synthase (ALS) activity, an herbicide-resistant glutamine synthetase (GS) activity, an herbicide-resistant 5-enol-pyruvyl-shikimate-3-phosphate synthase (EPSPS) activity, and any combination thereof. In some embodiments, the plant is selected from the group consisting of: a wheat plant, a maize plant, a rice plant, a barley plant, a sorghum plant, a rye plant, a canola plant, a broccoli plant, a cauliflower plant, a cotton plant, and a soybean plant. In some embodiments, the plant is the rice plant, and wherein the plant comprises the altered mitochondrion genome. In some embodiments, the plant is a rice plant, and wherein the plant comprises the altered plastid genome. In some embodiments, the polynucleotide encoding the SDN is stably integrated into a nuclear genome of the cell. In some embodiments, the SDN is a modified SDN. In some embodiments, the SDN comprises a mitochondrial targeting amino acid sequence (MTS). In some embodiments, the SDN is selected from the group consisting of: a polynucleotide guided nuclease, a TALEN, a zinc-finger nuclease, a meganuclease, a restriction enzyme, and any combination thereof. In some embodiments, the SDN cleaves and degrades a mitochondrial DNA lacking the internal polynucleotide. In some embodiments, the target sequence is not present in the heterologous polynucleotide. In some embodiments, the polynucleotide encoding the SDN encodes a TALEN, wherein the TALEN comprises a first polypeptide and a second polypeptide, wherein each of the first polypeptide and the second polypeptide comprises an MTS and a nuclease domain capable of forming a dimer. In some embodiments, the first polypeptide comprises a first MTS, and the second polypeptide comprises a second MTS. In some embodiments, the first MTS and the second MTS are the same. In some embodiments, the first MTS and the second MTS are different. In some embodiments, the SDN comprises a plastid targeting amino acid sequence (PTS). In some embodiments, the SDN is selected from the group consisting of: a polynucleotide guided nuclease, a TALEN, a zinc-finger nuclease, a meganuclease, a restriction enzyme, and any combination thereof, and further wherein the SDN targets a sequence present in the endogenous polynucleotide and not present in the heterologous polynucleotide. In some embodiments, the polynucleotide encoding the SDN encodes a TALEN, wherein the TALEN comprises a first polypeptide and a second polypeptide, wherein each of the first polypeptide and the second polypeptide comprises a PTS and a nuclease domain capable of forming a dimer. In some embodiments, the first polypeptide comprises a first PTS, and the second polypeptide comprises a second PTS. In some embodiments, the first PTS and the second PTS are the same. In some embodiments, the first PTS and the second PTS are different. In some embodiments, at least about 60% of mitochondrial genomes of the plurality of mitochondrial genomes or at least about 60% of plastid genomes of the plurality of plastid genomes in the cell comprise the internal polynucleotide. In some embodiments, at least about 70% of mitochondrial genomes of the plurality of mitochondrial genomes or at least about 70% of plastid genomes of the plurality of plastid genomes in the cell comprise the internal polynucleotide. In some embodiments, at least about 80% of mitochondrial genomes of the plurality of mitochondrial genomes or at least about 80% of plastid genomes of the plurality of plastid genomes in the cell comprise the internal polynucleotide. In some embodiments, at least about 90% of mitochondrial genomes of the plurality of mitochondrial genomes or at least about 90% of plastid genomes of the plurality of plastid genomes in the cell comprise the internal polynucleotide. In some embodiments, at least about 95% of mitochondrial genomes of the plurality of mitochondrial genomes or at least about 95% of plastid genomes of the plurality of plastid genomes in the cell comprise the internal polynucleotide. In some embodiments, the plant or cell disclosed herein can be provided in a kit with instructions for use.

Provided herein is a polypeptide comprising a transcription activator-like (TAL) effector DNA-binding domain and a functional nuclease domain, wherein the functional nuclease domain comprises or consists of an amino acid sequence that is at least about 70% identical to an amino acid sequence of SEQ ID NO: 5 or SEQ ID NO: 60. In some embodiments, the functional nuclease domain comprises or consists of an amino acid sequence of SEQ ID NO: 5 or SEQ ID NO: 60. In some embodiments, the functional nuclease domain is derived from an endonuclease, wherein the endonuclease comprises or consists of an amino acid sequence that is at least about 70% identical to an amino acid sequence of SEQ ID NO: 4. In some embodiments, the endonuclease comprises or consists of SEQ ID NO: 4. In some embodiments, the functional nuclease domain is fused to a carboxy-terminus of the TAL effector DNA-binding domain. In some embodiments, the functional nuclease domain is fused to an amino-terminus of the TAL effector DNA-binding domain. In some embodiments, two copies of the functional nuclease domain are fused to the carboxy-terminus of the TAL effector DNA-binding domain. In some embodiments, two copies of the functional nuclease domain are fused to the amino-terminus of the TAL effector DNA-binding domain. Provided herein is a polynucleotide encoding the polypeptide described herein. Provided herein is a plant cell comprising the polynucleotide described herein. Provided herein is a plant comprising the plant cell described herein. In some embodiments, the plant is selected from the group consisting of: a wheat plant, a maize plant, a rice plant, a barley plant, a sorghum plant, a rye plant, a canola plant, a broccoli plant, a cauliflower plant, a cotton plant, and a soybean plant. In some embodiments, the TAL effector DNA-binding domain and/or the functional nuclease domain disclosed herein can be provided in a kit with instructions for use.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a duplex PCR analysis of the regions specific to Donor DNA and to wild-type rice mitochondrial DNA. Two independent PCR reactions were performed for each sample. Callus DNA was used from the indicated transgenic events. Lanes 1 & 2: wild-type rice DNA; Lanes 3 & 4: AA1 event; Lanes 5 & 6: wild-type rice DNA; Lanes 7 & 8: AA5 event; Lanes 9 & 10: AA9 event; and Lanes 11 & 12; C97 event. The PCR-amplified DNA corresponding to Donor DNA (318 bp) and wild-type mitochondrial DNA (205 bp) are indicated with arrows.

FIG. 2 shows a duplex PCR analysis of the regions specific to Donor DNA and wild-type mitochondrial DNA. Two independent PCR reactions were performed for each sample. Leaf or callus DNA from the transgenic events were used as indicated. Lanes 1 & 2: wild-type rice DNA; Lanes 3 & 4: leaf DNA from event HH43; Lanes 5 & 6: callus DNA from event GG61; Lanes 7 & 8: callus DNA from event GG62; Lanes 9 & 10: callus DNA from event GG63; Lanes 11 & 12: callus DNA from event GG65; and Lanes 13 & 14: callus DNA from event C75. The PCR-amplified DNA corresponding to Donor DNA (318 bp) and wild-type mitochondrial DNA (205 bp) are indicated with arrows.

FIG. 3 shows an example duplex PCR analysis of the regions specific to Donor DNA and to wild-type rice mitochondrial DNA in three regenerated T0 plants. Lane 1: Control plasmid pNAP974 that contains both the Donor DNA fragment and the wild-type DNA fragment that is replaced upon transformation; Lane 2: wild-type rice DNA; Lane 3: DNA from young leaf of plant #1; Lane 4: DNA from older leaf of plant #1; Lane 5: DNA from young leaf of plant #2; Lane 6: DNA from older leaf of plant #2; Lane 7: DNA from young leaf of plant #3; Lane 8: DNA from older leaf of plant #3. The PCR-amplified DNAs corresponding to Donor DNA (318 bp) and wild-type mitochondrial DNA (205 bp) are indicated with arrows.

FIG. 4 shows three examples of regenerated T0 transgenic plants. Rice callus was initially transformed via the bombardment method with pNAP432 (described in Example 2) and pNAP159 (described in Example 1). After antibiotic selection with sulfonylurea and hygromycin, a transgenic callus event designated AO29 was secondarily transformed via the Agrobacterium method with pNAP651, a plasmid containing TALENs having a novel NAP12 nuclease domain. Three T0 plants were regenerated from the Agrobacterium-transformed callus designated AX13. #1: T0 plant designated 72.1.3.4; #2: T0 plant designated 72.1.3.5; #3: T0 plant designated 72.1.3.6.

### DETAILED DESCRIPTION

In some cases, mitochondrial genome editing and/or plastid genome editing can be more challenging to accomplish than nuclear genome editing. In some cases, disclosed herein are methods for editing mitochondrial or plastid genome. In some cases, site-directed nucleases can be used to generate and identify a cell comprising a plurality of edited mitochondrial genomes or edited plastid genomes. In some cases, site-directed nucleases can be used to generate a plant that is homoplasmic for an edited mitochondrial genome or an edited plastid genome.

Disclosed herein are methods for replacing an endogenous polynucleotide present in a genome of a mitochondrion or a plastid of a cell. In some embodiments, the method comprises: (a) introducing into the mitochondrion or the plastid of the cell a replacement DNA comprising in the 5′-to-3′ direction: (i) a 5′-homologous region (5′-HR) polynucleotide; (ii) an internal polynucleotide; and (iii) a 3′-homologous region (3′-HR) polynucleotide; wherein the internal polynucleotide comprises a sequence heterologous to the genome of the mitochondrion or the plastid, wherein the 5′-HR polynucleotide comprises a sequence with homology to a genomic region adjacent to one end of the endogenous polynucleotide to be replaced, and further wherein the 3′-HR polynucleotide comprises a sequence with homology to a genomic region adjacent to the other end of the endogenous polynucleotide to be replaced; (b) introducing into the cell a modified site-directed nuclease, wherein the modified site-directed nuclease comprises a site-directed nuclease (SDN) operably linked to a mitochondrial targeting amino acid sequence or a plastid targeting amino acid sequence, wherein the SDN recognizes a target sequence in the endogenous polynucleotide to be replaced; and (c) selecting a modified cell, wherein of the plurality of mitochondrial genomes or the plurality of plastid genomes, at least 50% comprise the internal polynucleotide and less than 50% comprise the endogenous polynucleotide to be replaced. In some embodiments, the replacement DNA can be introduced either before, at the same time, or after the introduction of a site-directed nuclease.

### Definitions

In some embodiments, the meaning of abbreviations can be as follows: “sec” can mean second(s), “min” can mean minute(s), “h” can mean hour(s), “d” can mean day(s), “μL” can mean microliter(s), “ml” can mean milliliter(s), “L” can mean liter(s), “μM” can mean micromolar, “mM” can mean millimolar, “M” can mean molar, “mmol” can mean millimole(s), “μmole” can mean micromole(s), “g” can mean gram(s), “μg” can mean microgram(s), “ng” can mean nanogram(s), “U” can mean unit(s), “nt” can mean nucleotide(s); “bp” can mean base pair(s), “kb” can mean kilobase(s) and “kbp” can mean kilobase pair(s).

In some embodiments, “transgenic” can refer to any cell, cell line, callus, tissue, organism part or whole organism (e.g., plant), the genome of which has been edited or altered by the presence of a heterologous or exogenous nucleic acid, such as a recombinant DNA construct. In some embodiments, transgenic events can include those created by sexual crosses or asexual propagation. In some embodiments, the term “transgenic” may not encompass an edited genome or alteration of a genome (e.g., chromosomal, or extra-chromosomal) by breeding methods or by naturally occurring events such as random cross-fertilization, non-recombinant viral infection, non-recombinant bacterial transformation, non-recombinant transposition, or spontaneous mutation. In some embodiments, the term “transgenic” may encompass an edited genome or alteration of a genome (e.g., chromosomal, or extra-chromosomal) by breeding methods or by naturally occurring events such as random cross-fertilization, non-recombinant viral infection, non-recombinant bacterial transformation, non-recombinant transposition, or spontaneous mutation.

In some embodiments, “genome”, for example, of a cell or whole organism can encompass chromosomal DNA found within a nucleus (nuclear DNA), and DNA found within a cytoplasmic organelle (e.g., mitochondrial DNA, plastid DNA). Methods and compositions described herein can be used for editing the genome of a nucleus, a cytoplasmic organelle (e.g., mitochondrion or plastid), or any combination thereof.

In some embodiments, “polynucleotide”, “nucleic acid”, “nucleic acid sequence”, “nucleotide sequence”, or “nucleic acid fragment”, which can be used interchangeably, can refer to a polymer of a nucleic acid (e.g., RNA, DNA, or both, and analogs thereof) that can be single-stranded or double-stranded (or both single-stranded and double-stranded), optionally containing synthetic, non-natural or altered nucleotide bases. In some embodiments, nucleotides (e.g., in their 5′-monophosphate form) can be referred to by a single letter designation as follows (for RNA or DNA, respectively): “A” for adenylate or deoxyadenylate, “C” for cytidylate or deoxycytidylate, “G” for guanylate or deoxyguanylate, “U” for uridylate, “T” for deoxythymidylate, “R” for purine-based nucleotides (A or G), “Y” for pyrimidine-based nucleotides (C or T), “K” for G or T, “H” for A or C or T, “I” for inosine, and “N” for any nucleotide. In some embodiments, a polynucleotide can be linear or circular.

In some embodiments, as used herein, a “nucleic acid” can refer to a polynucleotide sequence, or fragment thereof. In some embodiments, a nucleic acid can comprise nucleotides. In some embodiments, a nucleic acid can exist in a cell-free environment. In some embodiments, a nucleic acid can be a gene or fragment thereof. In some embodiments, a nucleic acid can be DNA. In some embodiments, a nucleic acid can be RNA. In some embodiments, a nucleic acid can comprise one or more analogs (e.g., altered backbone, sugar, or nucleobase). In some embodiments, non-limiting examples of analogs can include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine.

In some embodiments, “polypeptide”, “peptide”, “amino acid sequence” and “protein”, which can be used interchangeably herein, can refer to a polymer of amino acid residues. In some embodiments, these terms can apply to amino acid polymers in which one or more amino acid residues can be, for example, an artificial chemical analogue of a corresponding naturally occurring amino acid and/or to naturally occurring amino acid polymers. In some embodiments, the terms “polypeptide”, “peptide”, “amino acid sequence”, and “protein” can be inclusive of modifications including, but not limited to, glycosylation, lipid attachment, sulfation, gamma-carboxylation of glutamic acid residues, hydroxylation and ADP-ribosylation. In some embodiments, the polypeptide having an enzymatic activity can comprise an active site that facilitates enzymatic activity. In some embodiments, the active site of the polypeptide having an enzymatic activity can comprise a substrate binding domain and a catalytic domain that catalyze a reaction of the substrate.

In some embodiments, a “functional fragment” of a polynucleotide or polypeptide can refer to any subset of contiguous nucleotides or contiguous amino acids, respectively, in which an original (e.g., wild type) activity (or substantially similar activity) of a polynucleotide or polypeptide can be retained. In some embodiments, the terms “functional fragment”, “functional subfragment”, “fragment that is functionally equivalent”, “subfragment that is functionally equivalent”, “functionally equivalent fragment”, “a biologically active fragment” and “functionally equivalent subfragment” can be used interchangeably herein.

In some embodiments, the terms “variant”, “functional variant”, “variant that is functionally equivalent” and “functionally equivalent variant” can be used interchangeably herein. In some embodiments, in the context of a polynucleotide or a polypeptide, these terms can refer to a variant of the nucleic acid sequence or the amino acid sequence, respectively, in which the original activity (or substantially similar activity) of the polynucleotide or polypeptide can be retained. In some embodiments, fragments and variants can be obtained via methods such as site-directed mutagenesis and synthetic construction.

In some embodiments, an activity of a functional fragment or functional variant can be, for example, about: 100%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, or less than 10% of that of an original (e.g., wild type) activity.

In some embodiments, an “RNA transcript” can refer to a product resulting from an RNA polymerase-catalyzed transcription of a DNA sequence. In some embodiments, when an RNA transcript is a perfect complementary copy of a DNA sequence, it can be referred to as a primary transcript. In some embodiments, an RNA transcript can be referred to as a mature RNA, for example, when it is an RNA sequence derived from post-transcriptional processing of a primary transcript.

In some embodiments, a “messenger RNA” or “mRNA” can refer to an RNA that is without introns and that can be translated into protein by a cell.

In some embodiments, “sense” RNA can refer to an RNA transcript that includes an mRNA. In some embodiments, sense RNA can be translated into protein within a cell or in vitro.

In some embodiments, “antisense RNA” can refer to an RNA transcript that can be complementary to all or part of a target RNA (e.g., a primary transcript or mRNA). In some embodiments, antisense RNA can be used to block expression of a target gene. In some embodiments, a complementarity of an antisense RNA may be with any part of a specific gene transcript, i.e., at a 5′ non-coding sequence, 3′ non-coding sequence, introns, or a coding sequence.

In some embodiments, “functional RNA” can refer to antisense RNA, ribozyme RNA, ribosomal RNA (rRNA), transfer RNA (tRNA), or other RNA that may not be translated but yet can have an effect on cellular processes.

In some embodiments, the terms “complement” and “reverse complement” can be used interchangeably herein, for example, with respect to mRNA transcripts and can be used to define the antisense RNA of a message.

In some embodiments, a “coding region” can refer to a portion of a messenger RNA (or a corresponding portion of another nucleic acid molecule such as a DNA molecule) which can encode a protein or polypeptide. In some embodiments, a “non-coding region” can refer to a portion of a messenger RNA or other nucleic acid molecule that is not a coding region, including but not limited to, for example, a promoter region, a 5′ untranslated region (“UTR”), a 3′ UTR, an intron, and a terminator. In some embodiments, the terms “coding region” and “coding sequence” can be used interchangeably herein. In some embodiments, the terms “non-coding region” and “non-coding sequence” can be used interchangeably herein.

In some embodiments, “coding sequence” can be abbreviated “CDS”. In some embodiments, “open reading frame” can be abbreviated “ORF”.

In some embodiments, “gene” can refer to a nucleic acid fragment that can express a functional molecule such as, but not limited to, a specific protein, including: introns, exons, regulatory sequences preceding (5′ non-coding sequences) and following (3′ non-coding sequences) a coding sequence. In some embodiments, “native gene” can refer to a gene as found in nature, for example, with its own regulatory sequences.

In some embodiments, a “mutated gene” can be a gene that has been altered relative to a corresponding naturally occurring gene; e.g., through human intervention. In some embodiments, such a “mutated gene” can have a sequence that differs from a sequence of a corresponding non-mutated gene by at least one nucleotide addition, deletion, or substitution. In some embodiments, a mutated gene can comprise an alteration that results from a polynucleotide guided polypeptide system as disclosed herein. In some embodiments, a mutated organism can be an organism comprising a mutated gene; e.g., a mutated plant with an organellar genome comprising a mutated gene. In some embodiments, the terms “mutated gene” and “mutant gene” can be used interchangeably herein.

In some embodiments, the term “SDN” can refer to “site-directed nuclease”. In some embodiments, an SDN-induced mutation can include; an induction of site-specific random mutations; an induction of mutations in a predefined sequence of a particular gene; a replacement or an insertion of an entire gene; or any combination thereof. In some embodiments, SDN-induced mutations can be referred to as SDN-1, SDN-2, and SDN-3, respectively.

In some embodiments, a “codon-modified gene” or “codon-preferred gene” or “codon-optimized gene” can be a gene having its frequency of codon usage designed to mimic a frequency of preferred codon usage of a host cell in a compartment of interest. In some embodiments, a compartment of interest can comprise a nucleus, a mitochondrion, a chloroplast, or any combination thereof.

In some embodiments, a “mature” protein can refer to a post-translationally processed polypeptide; for example, one from which any pre- or pro-peptides present in a primary translation product have been removed.

In some embodiments, a “precursor” protein can refer to a primary product of translation of an mRNA; for example, with pre- and pro-peptides still present. In some embodiments, pre- and pro-peptides may, for example, comprise intracellular localization signals.

In some embodiments, “isolated” can refer to materials, such as nucleic acid molecules, proteins, and cells that may be substantially free or otherwise removed from components that normally accompany or interact with materials in a naturally occurring environment. In some embodiments, isolated polynucleotides can be purified from a host cell in which they can naturally occur. In some embodiments, nucleic acid purification methods can be used to obtain isolated polynucleotides. In some embodiments, isolated polynucleotides can include, for example, recombinant polynucleotides and chemically synthesized polynucleotides.

In some embodiments, “heterologous”, for example, with respect to sequence, can mean a sequence that originates from a foreign species, or, if from the same species, is substantially modified from its native form in composition and/or genomic locus by deliberate human intervention. In some embodiments, the terms “heterologous nucleotide sequence”, “heterologous sequence”, “heterologous nucleic acid fragment”, and “heterologous nucleic acid sequence” can be used interchangeably herein. In some embodiments, “heterologous” can refer to a nucleic acid sequence which does not naturally occur in a genome. In some embodiments, “heterologous” can refer to a nucleic acid sequence which has been artificially introduced into a genome. In some embodiments, “heterologous” can refer to a nucleic acid sequence which is present in a genome as a result of genetic editing. In some embodiments, a heterologous nucleic acid can be exogenous or endogenous to a cell. In some embodiments, “heterologous” can refer to a nucleic acid sequence which does not naturally occur in one organelle of a cell, but which does naturally occur in another organelle of the same cell. In some embodiments, “heterologous” can refer to a nucleic acid sequence that has been introduced into a mitochondrion of a cell which does not naturally occur in the mitochondrion of that cell, but which does naturally occur in a nucleus of that cell. In some embodiments, methods disclosed herein can produce an organelle that comprises a heterologous nucleic acid sequence that has not been integrated into a genome of the organelle. In some embodiments, a heterologous nucleic acid sequence that has not been integrated into a genome of the organelle can comprise a sequence that is part of a plasmid.

In some embodiments, “exogenous”, for example, with respect to sequence, can mean a sequence that originates from a foreign species. In some embodiments, the terms “exogenous nucleotide sequence”, “exogenous sequence”, “exogenous nucleic acid fragment”, and “exogenous nucleic acid sequence” can be used interchangeably herein. In some embodiments, “exogenous” can refer to a nucleic acid sequence which does not naturally occur in a genome. In some embodiments, “exogenous” can refer to a nucleic acid sequence which has been artificially introduced into a genome.

In some embodiments, “recombinant” can refer to an artificial combination of two or more otherwise separated segments of sequence, e.g., by chemical synthesis or by a manipulation of isolated segments of nucleic acids by genetic engineering techniques. In some embodiments, “recombinant” can also include reference to a cell or vector, for example, that has been modified by an introduction of a heterologous nucleic acid or a cell derived from a cell so modified.

In some embodiments, a “recombinant DNA construct” can refer to a combination of nucleic acid fragments that may not normally be found together in nature. In some embodiments, a recombinant DNA construct may comprise, for example, regulatory sequences and coding sequences that are derived from different sources, or regulatory sequences and coding sequences derived from the same source. In some embodiments, sequences in a recombinant DNA construct can be arranged in a manner different than that normally found in nature. In some embodiments, the terms “recombinant DNA construct”, “recombinant DNA molecule”, “recombinant construct”, “DNA construct” and “construct” can be used interchangeably herein. In some embodiments, a recombinant DNA construct may be any of the following non-limiting examples: single-stranded, double-stranded, or both single-stranded and double-stranded; linear or circular; DNA, RNA, or a combination of DNA and RNA; a plasmid DNA, a viral DNA, a viral RNA, or a viroid RNA.

In some embodiments, “expression” can refer to a production of a functional product. For example, expression of a nucleic acid fragment may refer to transcription of the nucleic acid fragment (e.g., transcription resulting in mRNA or functional RNA) and/or translation of mRNA into a precursor or mature protein.

In some embodiments, an “expression cassette” can refer to a construct containing, for example, a polynucleotide, a regulatory element(s), and a polynucleotide that allow for expression of a polynucleotide in a host. In some embodiments, the terms “expression cassette” and “expression construct” can be used interchangeably herein.

In some embodiments, the terms “entry clone” and “entry vector” can be used interchangeably herein.

In some embodiments, “regulatory sequences” can refer to nucleotide sequences, for example, located upstream (e.g., 5′ non-coding sequences), within (e.g., in introns), or downstream (e.g., 3′ non-coding sequences) of a coding sequence. In some embodiments, regulatory sequences can influence, for example, the transcription, RNA processing or stability, or translation of the associated coding sequence. In some embodiments, regulatory sequences may include, but are not limited to, promoters, translation leader sequences, 5′ untranslated sequences, 3′ untranslated sequences, introns, polyadenylation target sequences, RNA processing sites, effector binding sites, and stem-loop structures. In some embodiments, a regulatory sequence may act in “cis” or “trans”. In some embodiments, the nucleic acid molecule regulated by a regulatory sequence may not necessarily have to encode a functional peptide or polypeptide, e.g., the regulatory sequence can modulate the expression of a short interfering RNA or an antisense RNA. In some embodiments, the terms “regulatory sequence” and “regulatory element” can be used interchangeably herein.

In some embodiments, “promoter” can refer to a nucleic acid fragment that can control transcription of another nucleic acid fragment. In some embodiments, a promoter can include a core promoter (also known as minimal promoter) sequence. In some embodiments, a core promoter can be a minimal sequence for direct transcription initiation. In some embodiments, a core promoter can optionally include enhancers or other regulatory elements. In some embodiments, promoters may be derived in their entirety from a native gene, or be composed of different elements derived from different promoters found in nature, or even comprise synthetic DNA segments. Different promoters may direct the expression of a gene in different tissues or cell types, or at different stages of development, or in response to different environmental conditions.

In some embodiments, a “promoter functional in a plant” can be a promoter that can control transcription in plant cells. In some embodiments, a promoter can be from any suitable origin, which can include plant cells and non-plant cells.

In some embodiments, a “tissue-specific promoter” and “tissue-preferred promoter” can be used interchangeably and can refer to a promoter that can be expressed predominantly in one tissue, one organ or one cell type. In some embodiments, a tissue-specific promoter may not be necessarily exclusive in one tissue, one organ or one cell type. In some embodiments, a root-preferred promoter can include, for example, the following: soybean root-specific glutamine synthetase gene; cytosolic glutamine synthetase (GS); root-specific control element in the GRP 1.8 gene of French bean; root-specific promoter of A. tumefaciens mannopine synthase (MAS); root-specific promoters isolated from Parasponia andersonii and Trema tomentosa; A. rhizogenes rolC and rolD root-inducing genes; Agrobacterium wound-induced TR1′ and TR2′ genes; VfENOD-GRP3 gene promoter; and roll promoter. In some embodiments, a seed-preferred promoter can include a seed-specific promoter active during seed development, a seed-germinating promoter active during seed germination, or any combination thereof. In some embodiments, a seed-preferred promoter can include Cim1 (cytokinin-induced message); cZ19B1 (maize 19 kDa zein); milps (myo-inositol-1-phosphate synthase); END1; and END2, or any combination thereof. In some embodiments, for a dicot, a seed-preferred promoter can include; bean β-phaseolin; napin; β-conglycinin; soybean lectin; cruciferin; and any combination thereof. In some embodiments, for monocots, a seed-preferred promoter can include maize 15 kDa zein; 22 kDa zein; 27 kDa gamma zein; waxy; shrunken 1; shrunken 2; globulin 1; oleosin; nud; Zea mays-Rootmet2 promoter, or any combination thereof. In some embodiments, a leaf-preferred promoter can include a plant rbcS promoter, such as a soybean rbcS promoter, a maize rbcS promoter; a Zea mays PEPC1 promoter, or any combination thereof.

In some embodiments, an “inducible promoter” can refer to a promoter that selectively expresses an operably linked DNA sequence in response to a presence of an endogenous or exogenous stimulus, for example by a chemical compound (e.g., a chemical inducer) or in response to an environmental, hormonal, chemical, and/or developmental signal. In some embodiments, an inducible or regulated promoter can include, for example, promoters regulated by light, heat, stress, flooding, or drought, phytohormones, wounding, or chemicals such as ethanol, jasmonate, salicylic acid, or safeners. In some embodiments, a pathogen-inducible promoter that can be induced following infection by a pathogen can include, those regulating expression of PR proteins, SAR proteins, beta-1,3-glucanase, chitinase, or any combination thereof. In some embodiments, a stress-inducible promoter can include a plant RAB17 promoter, such as a maize RAB17 promoter. In some embodiments, a chemical-inducible promoter can include a maize ln2-2 promoter; a maize GST promoter; a tobacco PR-1a promoter, or any combination thereof. In some embodiments, a maize ln2-2 promoter can be activated by benzene sulfonamide herbicide safeners. In some embodiments, a maize GST promoter can be activated by a hydrophobic electrophilic compound. In some embodiments, a maize GST promoter can be used as a pre-emergent herbicide. In some embodiments, a tobacco PR-1a promoter can be activated by salicylic acid. In some embodiments, a chemical-regulated promoter can include a steroid-responsive promoter, for example, a glucocorticoid-inducible promoter, a tetracycline-inducible and a tetracycline-repressible promoter.

In some embodiments, a “constitutive promoter” can refer to promoters active in all or most tissues or cell types of an organism at all or most developing stages. In some embodiments, a promoter classified as “constitutive” (e.g., ubiquitin), some variation in absolute levels of expression can exist among different tissues or stages. In some embodiments, the term “constitutive promoter” or “tissue-independent promoter” can be used interchangeably herein. In some embodiments, constitutive promoters include the following: the core promoter of the Rsyn7 promoter; the core CaMV 35S promoter; plant actin promoter, such as a rice actin promoter and a maize actin promoter; plant ubiquitin promoter, such as a maize ubiquitin promoter and a soy bean ubiquitin promoter; pEMU; MAS promoter; ALS promoter; plant GOS2 promoter, such as a maize GOS2 promoter; soybean GM-EF1 A2 promoter; plant U6 polymerase III promoter, such as a maize U6 polymerase III promoter and a soybean U6 polymerase III promoter (GM-U6-9.1 and GM-U6-13.1); and any combination thereof.

In some embodiments, an enhancer element can be any nucleic acid molecule that increases transcription of a nucleic acid molecule when functionally linked to a promoter regardless of its relative position. In some embodiments, an enhancer may be an innate element of the promoter, or a heterologous element inserted to enhance the level or tissue-specificity of a promoter.

In some embodiments, a repressor (also sometimes called herein silencer) can be defined as any nucleic acid molecule which inhibits the transcription when functionally linked to a promoter regardless of relative position.

In some embodiments, a “transcription terminator”, “termination sequence”, or “terminator” can refer to DNA sequences that, when operably linked to the 3′ end of a polynucleotide sequence that is to be expressed, can terminate transcription from the polynucleotide sequence. In some embodiments, a transcription termination can refer to the process by which RNA synthesis by RNA polymerase can be stopped and both the RNA and the enzyme are released from the DNA template.

In some embodiments, “operably linked” can refer to the association of fragments in a single fragment (e.g., a polynucleotide or polypeptide), or in a single complex, so that the function of one can be regulated by the other. In some embodiments, a linkage may be covalent or non-covalent. In some embodiments, with respect to nucleic acid fragments, a promoter can be operably linked with a nucleic acid fragment if the promoter can regulate the transcription of that nucleic acid fragment. In some embodiments, with respect to a polypeptide, an organelle targeting peptide can be operably linked with a polypeptide if the organelle targeting peptide can transport that polypeptide into the relevant organelle. In some embodiments, with respect to a complex, a guide RNA can be operably linked to a Cas polypeptide if the guide RNA/Cas polypeptide complex can cleave a target sequence as directed by the guide RNA.

In some embodiments, a “phenotype” can refer to the detectable characteristics of a cell or organism.

In some embodiments, the term “introduced” can mean providing a polynucleic acid (e.g., expression construct) or protein into a cell. In some embodiments, “introduced” can include reference to the incorporation of a nucleic acid into a eukaryotic or prokaryotic cell, for example, where the nucleic acid may be incorporated into the genome of the cell. In some embodiments, “introduced” can include reference to the transient provision of a nucleic acid or protein to the cell. In some embodiments, “introduced” can include reference to stable or transient gene editing methods. In some embodiments, “introduced” can include reference to stable or transient transformation methods. Introduced can include sexually crossing. In some embodiments, “introduced”, for example, in the context of inserting a nucleic acid fragment (e.g., a recombinant DNA construct) into a cell, can include “transfection” or “transformation” or “transduction”. In some embodiments, “introduced” can include reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

In some embodiments, an “edited genome” may comprise introduction of (i) a substitution of at least one nucleotide, (ii) a deletion of at least one nucleotide (iii) an insertion of at least one nucleotide or (iv) any combination of (i)-(iii). In some embodiments, substitution and replacement are both used herein to mean the interchange of an existing nucleotide with an alternative nucleotide. In some embodiments, a cell may comprise an edited genome (e.g., of a mitochondrion, a plastid, or both) with at least one nucleotide substitution, deletion, or insertion. In some embodiments, a cell may comprise a transformed organelle (e.g., transformed mitochondrion, transformed plastid) wherein the transformed organelle comprises the edited genome.

In some embodiments, a “transformed cell” can be any cell in which a nucleic acid fragment (e.g., a recombinant DNA construct) has been introduced or edited.

In some embodiments, “transformation” as used herein can refer to a stable transformation. In some embodiments, a transformation can refer to transient transformation.

In some embodiments, “stable transformation” can refer to an introduction of a nucleic acid fragment into a genome (e.g., of the nucleus, mitochondrion, plastid) of a host organism resulting in genetically stable inheritance. In some embodiments, once stably transformed, the nucleic acid fragment can be stably integrated in the genome of the host organism and any subsequent generation.

In some embodiments, a “transient transformation” can refer to the introduction of a nucleic acid fragment into the nucleus, or DNA-containing cytoplasmic organelle (e.g., mitochondrion, plastid), thereby editing or modifying a host organism nucleus or organelle genomes resulting in gene expression without genetically stable inheritance.

In some embodiments, host organisms containing the transformed nucleic acid fragments can be referred to as “transgenic” organisms.

In some embodiments, a “transformation cassette” can refer to a construct having elements that facilitates transformation of a particular host cell. In some embodiments, the terms “transformation cassette” and “transformation construct” can be used interchangeably herein.

In some embodiments, “homoplasmic”, when used with respect to mitochondria, can refer to a eukaryotic cell in which the copies of mitochondrial DNA are all identical. In some embodiments, “heteroplasmic” can refer to a eukaryotic cell in which the copies of mitochondrial DNA are not all identical.

In some embodiments, “homoplasmic”, when used with respect to plastids, can refer to a eukaryotic cell in which the copies of plastid DNA are all identical. In some embodiments, “heteroplasmic” can refer to a eukaryotic cell in which the copies of plastid DNA are not all identical.

In some embodiments, the term “percent homoplasmy” when used with respect to mitochondria or plastids, can refer to a eukaryotic cell in which the percentage of the copies of mitochondrial DNA or plastid DNA, respectively, have a given change.

In some embodiments, an “allele” can be one of several alternative forms of a gene occupying a given locus on a chromosome. In some embodiments, when the alleles present at a given locus on a pair of homologous chromosomes in a diploid plant are the same that plant can be homozygous at that locus. In some embodiments, if the alleles present at a given locus on a pair of homologous chromosomes in a diploid plant differ, that plant can be heterozygous at that locus. In some embodiments, if a transgene is present on one of a pair of homologous chromosomes in a diploid plant that plant can be hemizygous at that locus.

In some embodiments, an “organelle” can be a DNA-containing organelle of the cell. In some embodiments, an organelle can be a nucleus, a mitochondrion, a plastid (e.g., a chloroplast), or any combination thereof. In some embodiments, an organelle can be a DNA-containing cytoplasmic organelle. In some embodiments, an organelle can be a mitochondrion, a plastid (e.g., a chloroplast), or any combination thereof. In some embodiments, a plastid can be a proplastid, an etioplast, a leucoplast, an amyloplast, an elaioplast, a proteinoplast, a chromoplast, a chloroplast, a geronoplast, or any combination thereof.

In some embodiments, the terms “organelle-specific” and “organelle-preferred” can be used interchangeably, and when used to describe a regulatory element (e.g., an organelle-specific promoter), refer to a regulatory element that is functional within a given cell (e.g., a plant cell) predominantly but not necessarily exclusively in an organelle (e.g., a mitochondrion, a plastid).

In some embodiments, an organelle-specific regulatory domain may be derived from an organellar polynucleotide of interest (e.g., a mitochondrial polynucleotide, a plastid polynucleotide). In some embodiments, an organelle-specific regulatory domain may comprise all or part of the nucleic acid sequence of an organellar polynucleotide of interest. In some embodiments, the organelle-specific regulatory domain may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the organellar polynucleotide of interest.

In some embodiments, the terms “mitochondrial-specific” and “mitochondrial-preferred” can be used interchangeably, and when used to describe a regulatory element (e.g., a mitochondrial-specific promoter), refer to a regulatory element that is functional within a given cell (e.g., a plant cell) predominantly but not necessarily exclusively in mitochondria.

In some embodiments, the terms “plastid-specific” and “plastid-preferred” can be used interchangeably, and when used to describe a regulatory element (e.g., a plastid-specific promoter), refer to a regulatory element that is functional within a given cell (e.g., a plant cell) predominantly but not necessarily exclusively in plastids.

In some embodiments, the terms “chloroplast-specific” and “chloroplast-preferred” can be used interchangeably, and when used to describe a regulatory element (e.g., a chloroplast-specific promoter), refer to a regulatory element that is functional within a given cell (e.g., a plant cell) predominantly but not necessarily exclusively in chloroplasts.

In some embodiments, the terms “mitochondrial genome” and “genome of a mitochondrion” can be used interchangeably and refer to the nucleic acid sequences present within endogenous mitochondrial genetic elements. In some embodiments, the mitochondrial genome may be edited by the addition of a sequence (e.g., a heterologous sequence) into an endogenous mitochondrial genetic element. In some embodiments, an autonomously replicating heterologous episomal element (e.g., a plasmid DNA) introduced into a mitochondrion is considered to be an independent genetic element and is not considered to be part of the mitochondrial genome.

In some embodiments, the terms “plastid genome”, “chloroplast genome”, “genome of a plastid” and “genome of a chloroplast” can be used interchangeably and refer to a nucleic acid sequence present within endogenous plastid genetic elements. In some embodiments, a plastid genome may be edited by the addition of a sequence (e.g., a heterologous sequence) into an endogenous plastid genetic element. In some embodiments, an autonomously replicating heterologous episomal element (e.g., a plasmid DNA) introduced into a plastid is considered to be an independent genetic element and is not considered to be part of the plastid genome.

In some embodiments, a “chloroplast transit peptide” can be an amino acid sequence that can direct a protein to the chloroplast or other plastid types present in the cell. In some embodiments, a chloroplast transit peptide can be translated in conjunction with the protein in the cell in which the protein can be made. In some embodiments, the terms “chloroplast transit peptide”, “plastid transit peptide”, “chloroplast targeting peptide” and “plastid targeting peptide” can be used interchangeably herein. “Chloroplast transit sequence” can refer to a nucleotide sequence that can encode a chloroplast transit peptide.

In some embodiments, a “signal peptide” can be an amino acid sequence that can direct a protein to the secretory system. The signal peptide can be translated in conjunction with a protein. For example, if the protein is to be directed to a vacuole, a vacuolar targeting signal (supra) can further be added, or if to an endoplasmic reticulum, an endoplasmic reticulum retention signal (supra) may be added. If a protein is to be directed to the nucleus, any signal peptide present can be removed and a nuclear localization signal can be included.

In some embodiments, a “mitochondrial targeting peptide” can be an amino acid sequence which can direct a precursor protein into the mitochondria. In some embodiments, the terms “mitochondrial targeting peptide”, “mitochondrial signal peptide” and “mitochondrial transit peptide” can be used interchangeably herein.

In some embodiments, an “organelle targeting polynucleotide” can be a nucleotide sequence which can direct import of the polynucleotide into an organelle. In some embodiments, the terms “organelle targeting polynucleotide”, “organelle targeting nucleic acid” and “organelle targeting nucleic acid sequence” can be used interchangeably herein. In some embodiments, an organelle targeting polynucleotide may be directed to, for example, the plastid (“plastid targeting polynucleotide”) or the mitochondria (“mitochondria targeting polynucleotide”). In some embodiments, a polynucleotide can be RNA (“organelle targeting RNA”), DNA (“organelle targeting DNA) or a combination of RNA and DNA. In some embodiments, an organelle targeting RNA directed to the plastid can be termed a “plastid targeting RNA”. In some embodiments, the terms “plastid targeting RNA”, “chloroplast targeting RNA” and “transit RNA” are used interchangeably herein. In some embodiments, an organelle targeting RNA directed to the mitochondria can be termed a “mitochondria targeting RNA”.

In some embodiments, RNAs can be imported into mitochondria. In some embodiments, one such mitochondrial targeting RNA can be the yeast tRNALys. In some embodiments, yeast tRNALys and its variants can be imported into human mitochondria. In some embodiments, another RNA that can be imported into mitochondria can be 5S rRNA. In some embodiments, 5S rRNA can function as a vector for delivering heterologous RNA sequences into, for example, mitochondria (e.g., human). In some embodiments, RNAs can be used with the compositions and methods of the disclosure for example, for targeting an organelle (e.g., the mitochondria).

In some embodiments, RNAs can be imported into plastids. In some embodiments, plastid targeting RNAs that can mediate import of attached heterologous RNA can include vd-5′UTR (e.g., viroid-derived ncRNA sequence acting as 5′UTR) and eIF4E1 mRNA. In some embodiments, RNAs can be used with the compositions and methods of the disclosure for targeting to an organelle (e.g., the plastid).

In some embodiments, as used herein, “fusion” can refer to a protein and/or nucleic acid comprising one or more non-native sequences (e.g., moieties). In some embodiments, any of the molecules described herein (e.g., nucleic acids, proteins, polypeptides, a polynucleic acid, a transcription activator-like effector nuclease (TALEN), a meganuclease, a restriction endonuclease, a Cas protein, a guide polynucleotide) can be engineered as fusions or as fusion proteins. In some embodiments, a fusion can comprise one or more of the same non-native sequences. In some embodiments, a fusion can comprise one or more of different non-native sequences. In some embodiments, a fusion can be a chimera. In some embodiments, a fusion can comprise a nucleic acid affinity tag. In some embodiments, a fusion can comprise a barcode. In some embodiments, a fusion can comprise a peptide affinity tag. In some embodiments, a fusion can provide for subcellular localization of the site-directed polypeptide. In some embodiments, a fusion can provide a non-native sequence (e.g., affinity tag) that can be used to track or purify. In some embodiments, a fusion can be a small molecule such as biotin or a dye such as alexa fluor dyes, Cyanine3 dye, Cyanine5 dye, or any combination thereof.

In some embodiments, a fusion can refer to any protein with a functional effect. In some embodiments, a fusion protein can comprise nuclease activity, nicking activity, deaminase activity, cytidine deaminase activity (US Patent Publication No. US20150166980, herein incorporated by reference), adenine deaminase activity (US Patent Publication No. US20180073012, herein incorporated by reference), uracil glycosylase inhibitor activity (US Patent Publication No. US20170121693, herein incorporated by reference), methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, or demyristoylation activity. In some embodiments, an effector protein can modify a genomic locus. In some embodiments, a fusion protein can be a fusion in a Cas protein. In some embodiments, a Cas protein can be a modified form that has nickase activity or that has no substantial nucleic acid-cleaving activity. In some embodiments, a fusion protein can be a non-native sequence in a Cas protein.

In some embodiments, “silencing”, as used herein with respect to the target gene, can refer to the suppression of levels of mRNA or protein/enzyme expressed by the target gene, and/or the level of the enzyme activity or protein functionality. In some embodiments, the terms “suppression”, “suppressing” and “silencing”, which can be used interchangeably herein, can include lowering, reducing, declining, decreasing, inhibiting, eliminating, or preventing. In some embodiments, “silencing” or “gene silencing” can occur by any suitable mechanism. In some embodiments, non-limiting examples of silencing can include antisense, co-suppression, viral-suppression, hairpin suppression, stem-loop suppression, RNAi-based approaches, small RNA-based approaches, and any combination thereof.

In some embodiments, suppression of gene expression can also be achieved by, for example, use of artificial miRNA precursors, ribozyme constructs and gene disruption. In some embodiments, a modified plant miRNA precursor may be used, wherein the precursor has been modified, for example, to replace the miRNA encoding region with a sequence designed to produce a miRNA directed to the nucleotide sequence of interest. In some embodiments, a gene disruption may be achieved by use of transposable elements or by use of chemical agents that cause site-specific mutations.

### Sequence Identity, Similarity, and Variation

In some embodiments, a sequence alignment and percent identity or similarity calculation may be determined using a variety of comparison methods designed to detect homologous sequences including, but not limited to, the MEGALIGN™ program of the LASERGENE™ bioinformatics computing suite (DNASTAR™ Inc., Madison, WI). In some embodiments, where sequence analysis software is used for analysis, results of an analysis can be based on “default values” of a program referenced. In some embodiments, as used herein “default values” can mean any set of values or parameters that originally load with the software when first initialized.

In some embodiments, “Clustal V method of alignment” can correspond to an alignment method labeled Clustal V and, for example, found in a MEGALIGN™ program of a LASERGENE™ bioinformatics computing suite (DNASTAR™ Inc., Madison, WI). In some embodiments, for multiple alignments, default values can correspond to GAP PENALTY=10 and GAP LENGTH PENALTY=10. In some embodiments, default parameters for pairwise alignments and calculation of percent identity of protein sequences using the Clustal method can be, for example, KTUPLE=1, GAP PENALTY=3, WINDOW=5 and DIAGONALS SAVED=5. In some embodiments, for nucleic acids these parameters can be for example KTUPLE=2, GAP PENALTY=5, WINDOW=4 and DIAGONALS SAVED=4. In some embodiments, after alignment of sequences using the Clustal V program, “percent identity” and “divergence” values can be obtained by viewing the “sequence distances” table in the same program.

In some embodiments, the “Clustal W method of alignment” can correspond to the alignment method labeled Clustal W and, for example, found in the MEGALIGN™ v6.1 program of the LASERGENE™ bioinformatics computing suite (DNASTAR™ Inc., Madison, WI). In some embodiments, default parameters for multiple alignment can correspond to for example: GAP PENALTY=10, GAP LENGTH PENALTY=0.2, Delay Divergence Sequences=30%, DNA Transition Weight-0.5, Protein Weight Matrix-Gonnet Series, DNA Weight Matrix=IUB. In some embodiments, after alignment of the sequences using the Clustal W program, “percent identity” values can be obtained by viewing the “sequence distances” table in the same program.

In some embodiments, sequence identity/similarity values can also be obtained using GAP Version 10 (GCG, ACCELRYS™, San Diego, CA) using for example the following parameters: % identity and % similarity for a nucleotide sequence using a gap creation penalty weight of 50 and a gap length extension penalty weight of 3, and the nwsgapdna. cmp scoring matrix; % identity and % similarity for an amino acid sequence using a GAP creation penalty weight of 8 and a gap length extension penalty of 2, and the BLOSUM62 scoring matrix. In some embodiments, GAP can use an algorithm to find an alignment of two complete sequences that can maximize the number of matches and minimize the number of gaps. In some embodiments, GAP can consider all possible alignments and gap positions. In some embodiments, GAP can create the alignment with the largest number of matched bases and the fewest gaps, using, for example, a gap creation penalty and a gap extension penalty in units of matched bases.

In some embodiments, “BLAST” can be a searching algorithm provided by the National Center for Biotechnology Information (NCBI) that can be used to find regions of similarity between biological sequences. In some embodiments, BLAST can compare nucleotide or protein sequences to sequence databases. In some embodiments, BLAST can calculate the statistical significance of matches to identify sequences having sufficient similarity to a query sequence such that the similarity may not be predicted to have occurred randomly. In some embodiments, BLAST can report the identified sequences and their local alignment to the query sequence.

In some embodiments, the term “conserved domain” or “motif” can mean a set of amino acids conserved at specific positions along an aligned sequence of evolutionarily related proteins. In some embodiments, while amino acids at other positions can vary between homologous proteins, amino acids that are highly conserved at specific positions can indicate, for example, amino acids that are essential to the structure, the stability, or the activity of a protein.

In some embodiments, conserved domains or motifs can be identified by their high degree of conservation in aligned sequences of a family of protein homologues. In some embodiments, conserved domains can be used as identifiers, or “signatures”, for example, to determine if a protein with a newly determined sequence belongs to a previously identified protein family.

In some embodiments, polynucleotide and polypeptide sequences, variants thereof, and the structural relationships of these sequences can be described by the terms “homology”, “homologous”, “substantially identical”, “substantially similar” and “corresponding substantially” which are used interchangeably herein. In some embodiments, these can refer to polypeptide or nucleic acid fragments wherein changes in one or more amino acids or nucleotide bases may not affect the function of the molecule, such as the ability to mediate gene expression or to produce a certain phenotype. In some embodiments, these terms can also refer to modification(s) of nucleic acid fragments that may not substantially alter the functional properties of the resulting nucleic acid fragment relative to the initial, unmodified fragment. In some embodiments, these modifications can include deletion, substitution, insertion, or any combination thereof, of one or more nucleotides in the nucleic acid fragment.

In some embodiments, substantially similar nucleic acid sequences encompassed may be defined by their ability to hybridize (for example, under moderately stringent conditions, e.g., 0.5×SSC, 0.1% SDS, 60° C.) with the sequences exemplified herein, or to any portion of the nucleotide sequences disclosed herein. In some embodiments, substantially similar nucleic acid sequences can be functionally equivalent to any of the nucleic acid sequences disclosed herein. In some embodiments, stringency conditions can be adjusted to screen for moderately similar fragments, such as homologous sequences from distantly related organisms to highly similar fragments, such as genes that duplicate functional enzymes from closely related organisms. In some embodiments, post-hybridization washes can determine stringency conditions.

In some embodiments, the term “selectively hybridizes” can include reference to hybridization, for example under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. In some embodiments, selectively hybridizing sequences can have, for example, about at least 80% sequence identity, or 90% sequence identity, up to and including 100% sequence identity (i.e., fully complementary) with each other.

In some embodiments, the term “stringent conditions” or “stringent hybridization conditions” can include reference to conditions under which a probe can selectively hybridize to its target sequence in an in vitro hybridization assay. In some embodiments, stringent conditions can be sequence-dependent. In some embodiments, stringent conditions can be different in different circumstances. In some embodiments, by controlling the stringency of the hybridization and/or washing conditions, target sequences can be identified which are 100% complementary to the probe (homologous probing).

In some embodiments, stringency conditions can be adjusted to allow some mismatching in sequences so that lower degrees of similarity are detected (heterologous probing). In some embodiments, a probe can be less than about 1000 nucleotides in length, optionally less than 500 nucleotides in length.

In some embodiments, stringent conditions can comprise those in which a salt concentration is less than about 1.5 M Na ion. In some embodiments, stringent conditions can comprise those in which a salt concentration is less than about 0.01 to 1.0 M Na ion concentration (or other salt(s)) at pH 7.0 to 8.3. In some embodiments, stringent conditions can comprise a temperature of about 30° C. for short probes (e.g., 10 to 50 nucleotides). In some embodiments, stringent conditions can comprise a temperature of at least about 60° C. for long probes (e.g., greater than 50 nucleotides). In some embodiments, stringent conditions can also be achieved with the addition of destabilizing agents such as formamide. In some embodiments, exemplary low stringency conditions can include hybridization with a buffer solution of, for example, 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. In some embodiments, exemplary moderate stringency conditions can include hybridization in 40 to 45% formamide, 1 M NaCl, 1% SDS at 37° C., and a wash in 0.5× to 1×SSC at 55 to 60° C. In some embodiments, exemplary high stringency conditions can include hybridization in, for example, 50% formamide, 1 M NaCl, 1% SDS at 37° C., and a wash in 0.1×SSC at 60 to 65° C.

In some embodiments, “sequence identity” or “identity” in the context of nucleic acid or polypeptide sequences can refer to the nucleic acid bases or amino acid residues in two sequences that are the same when aligned for maximum correspondence over a specified comparison window.

In some embodiments, the term “percentage of sequence identity” can refer to a value determined by comparing two optimally aligned sequences over a comparison window. In some embodiments, a portion of the polynucleotide or polypeptide sequence in the comparison window may comprise additions or deletions (i.e., gaps) as compared to the reference sequence (which may or may not comprise additions or deletions) for optimal alignment of the two sequences. In some embodiments, a percentage can be calculated by, for example, determining a number of positions at which an identical nucleic acid base or amino acid residue occurs in both sequences to yield a number of matched positions, dividing a number of matched positions by a total number of positions in a window of comparison and multiplying the results by 100 to yield the percentage of sequence identity. In some embodiments, percent sequence identities can include, but are not limited to, 50%, 55%, 60%, 65%, 70%, 75%, 80%, 85%, 90% or 95%, or any percentage from 50% to 100%. In some embodiments, sequence identity can include an integer percentage from 50% to 100%. In some embodiments, these identities can be determined using any of the programs described herein.

In some embodiments, sequence identity can be useful in identifying polypeptides from other species or modified naturally or synthetically wherein such polypeptides have the same or similar function or activity. In some embodiments, percent identities can include, but are not limited to, 50%, 55%, 60%, 65%, 70%, 75%, 80%, 85%, 90% or 95%. In some embodiments, sequence identity (e.g., amino acid sequence identity) can include an integer percentage from 50% to 100%. In some embodiments, sequence (e.g., amino acid) identity can include, for example, about: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

### Definitions, Traits, and Processes Relevant to Plants

In some embodiments, “plant” can include reference to whole plants, plant organs, plant tissues, plant propagules, seeds and plant cells and progeny of the same. In some embodiments, plant cells include, without limitation, cells from seeds, suspension cultures, embryos, meristematic regions, callus tissue, leaves, roots, shoots, gametophytes, sporophytes, pollen, and microspores.

In some embodiments, a “propagule” can include products of meiosis and/or mitosis able to propagate a new plant. In some embodiments, a propagule can include seeds, spores and parts of a plant that can serve as a means of vegetative reproduction, such as corms, tubers, offsets, or runners. In some embodiments, a propagule can include grafts where one portion of a plant can be grafted to another portion of a different plant (even one of a different species) to create a living organism. In some embodiments, a propagule can include plants and seeds produced by cloning or by bringing together meiotic products, or allowing meiotic products to come together to form an embryo or fertilized egg (naturally or with human intervention).

In some embodiments, a “progeny” can comprise any subsequent generation of a plant.

In some embodiments, the terms “monocot” and “monocotyledonous plant” can be used interchangeably herein. In some embodiments, a monocot can include the Gramineae.

In some embodiments, the terms “dicot” and “dicotyledonous plant” can be used interchangeably herein. In some embodiments, a dicot can include, for example, the following families: Brassicaceae, Leguminosae, and Solanaceae.

In some embodiments, “transgenic plant” can include reference to a plant which can comprise within its genome a heterologous polynucleotide. In some embodiments, a heterologous polynucleotide can be stably integrated within a genome (e.g., nuclear, plastid, mitochondrial) such that a polynucleotide can be passed on to successive generations. In some embodiments, a heterologous polynucleotide can be integrated into a genome alone or as part of a recombinant DNA construct.

In some embodiments, a “transgenic plant” can include reference to plants which can comprise more than one heterologous polynucleotide within their genome. In some embodiments, each heterologous polynucleotide can confer a different trait to a transgenic plant.

In some embodiments, multiple traits can be introduced into crop plants, and can be referred to as a gene stacking approach. In some embodiments, gene stacking can be used, for example, for development of genetically improved germplasm. In some embodiments, multiple genes conferring different characteristics of interest can be introduced into a plant. In some embodiments, gene stacking can be accomplished by many means including but not limited to co-transformation, retransformation, and crossing lines with different transgenes. In some embodiments, as used herein, the term “stacked” can include having multiple traits present in the same plant (e.g., both traits are incorporated into the nuclear genome, one trait is incorporated into the nuclear genome and one trait is incorporated into the genome of an organelle, or both traits are incorporated into the genome of an organelle).

In some embodiments, the term “crossed” or “cross” or “crossing” in the context of the disclosure can mean the fusion of gametes (e.g., via pollination) to produce progeny (e.g., cells, seeds, or plants). In some embodiments, the term can encompass both sexual crosses (e.g., the pollination of one plant by another) and selfing (e.g., self-pollination; when the pollen and ovule are from the same plant or genetically identical plants).

In some embodiments, the term “maternal inheritance” can refer to the transmission of traits that can be solely dependent on properties of the genome of the female gamete.

In some embodiments, the term “paternal inheritance” can refer to the transmission of traits that are solely dependent on properties of the genome of the male gamete.

In some embodiments, the term “introgression” can refer to the transmission of a desired allele of a genetic locus from one genetic background to another. In some embodiments, introgression of a desired allele at a specified locus can be transmitted to at least one progeny plant via a sexual cross between two parent plants, where at least one of the parent plants has the desired allele within its genome. In some embodiments, transmission of an allele can occur by recombination between two donor genomes, e.g., in a fused protoplast, where at least one of the donor protoplasts has the desired allele in its genome. In some embodiments, a desired allele can be, e.g., a transgene or a selected allele of a marker or QTL.

In some embodiments, “a plant-optimized nucleotide sequence” can be a nucleotide sequence that has been optimized for increased expression in plants, particularly for increased expression in a given plant or in one or more plants of interest. In some embodiments, a plant-optimized nucleotide sequence can be synthesized by modifying a nucleotide sequence encoding a protein by using plant-preferred codons for improved expression. In some embodiments, a host-preferred codon usage can be utilized for codon optimization. In some embodiments, a frequency of codon usage can be designed to mimic the frequency of preferred codon usage of a host cell in a compartment of interest, e.g., a nucleus, a mitochondrion, or a chloroplast.

In some embodiments, plant-preferred genes can be synthesized. In some embodiments, additional sequence modifications can enhance gene expression in a plant host. In some embodiments, these can include, for example, elimination of any of the following: one or more sequences encoding spurious polyadenylation signals, one or more exon-intron splice site signals, one or more transposon-like repeats, and sequences that can be deleterious to gene expression. In some embodiments, a G-C content of a sequence may be adjusted, for example, to levels average for a given plant host, as calculated by reference to genes expressed in a host plant cell. In some embodiments, when possible, a sequence can be modified to avoid one or more predicted hairpin secondary mRNA structures. In some embodiments, “a plant-optimized nucleotide sequence” of a present disclosure can comprise one or more of such sequence modifications.

In some embodiments, a “trait” can refer to, for example, a physiological, morphological, biochemical, or physical characteristic of a plant or particular plant material or cell. In some instances, a characteristic can be visible to a human eye, such as seed or plant size, or can be measured by biochemical techniques, such as detecting a protein, starch, or oil content of seed or leaves, or by observation of a metabolic or physiological process, e.g. by measuring tolerance to water deprivation or particular salt or sugar concentrations, or by an observation of an expression level of a gene or genes, or by agricultural observations such as osmotic stress tolerance or yield.

In some embodiments, an “agronomic characteristic” can be a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

In some embodiments, an “effective concentration” of a selective agent or an herbicide can refer to the minimum concentration of the selective agent or the herbicide required to kill all non-transformed cells, or a portion thereof. In some embodiments, the selective agent and/or the herbicide is present at least at an effective concentration. In some embodiments, applying an effective concentration of the selective agent or the herbicide can facilitate proper enrichment, isolation, or selection of cells of interest. That is, cells possessing a particular genetic trait or genetic change can be more easily enriched, isolated, or selected when the selective agent or the herbicide is applied at an effective concentration. In some embodiments, application of an effective concentration of a selective agent or an herbicide to a non-transformed cell and a transformed cell kills or hinders the growth of the non-transformed cell or portion thereof, but does not kill or hinder the growth of the transformed cell or portion thereof.

### Genome Modification

Disclosed herein, in some embodiments, are compositions and methods that can be used, for genome modification of a target sequence in a genome (e.g., a nucleus, a plastid, or a mitochondrial genome) of an organism or cell (e.g., a plant or plant cell), for selecting the modified organism or cell, for gene editing, and for inserting a donor polynucleotide (e.g., a replacement nucleic acid comprising an internal polynucleotide) into the genome (e.g., a nucleus, a plastid, or a mitochondrial genome) of an organism or cell. In some embodiments, the methods and compositions can edit, modify, or replace an endogenous target polynucleotide present in a genome of a cell. In some embodiments, the methods and compositions described herein can edit, modify, or replace an endogenous target polynucleotide present in a genome of a mitochondrion or a plastid of a cell. In some embodiments, the methods and compositions described herein can edit, modify, or replace an endogenous target polynucleotide with a replacement nucleic acid or a portion thereof (e.g., an internal polynucleotide) in a genome of a mitochondrion or a plastid of a cell.

In some embodiments, methods disclosed herein can employ a site-directed nuclease. In some embodiments, a site-directed nuclease can introduce a single strand or double strand break at a specific target site into a genome of a cell. In some embodiments, a site-directed nuclease can provide for an effective system for modifying target sites within a genome of a plant, plant cell or seed. In some embodiments, the methods and compositions described herein may not require a site-directed nuclease or site-directed nuclease mediated a single strand or double strand break to replace an endogenous target polynucleotide with a replacement nucleic acid or a portion thereof (e.g., an internal polynucleotide). For example, in some embodiments, the methods and compositions described herein can replace an endogenous target polynucleotide present in a genome of a mitochondrion or a plastid of a cell with an internal polynucleotide, wherein the method can comprise introducing a replacement nucleic acid or a portion thereof (e.g., an internal polynucleotide) into organelle genomes by utilizing a homologous recombination, without a site-directed nuclease mediated single strand or double strand breaks.

In some embodiments, a variety of methods can be employed to further modify a target site to introduce a donor polynucleotide of interest (e.g., a replacement nucleic acid comprising an internal polynucleotide). In some embodiments, a nucleotide sequence to be edited (e.g., a nucleotide sequence of interest) can be located within or outside a target site that can be recognized by a site-directed nuclease.

Also disclosed herein are methods and compositions employing a site-directed nuclease for modification of multiple target sites within a genome of an organelle. Modification of multiple target sites within a genome of an organelle can facilitate a creation of a homoplasmic transformation event.

In some embodiments, the methods described herein can comprise (a) introducing into the mitochondrion or the plastid a replacement nucleic acid comprising an internal polynucleotide, (b) selecting an altered cell comprising at least one altered mitochondrion genome or at least one altered plastid genome comprising the replacement nucleic acid or a portion thereof (e.g., an internal polynucleotide). In some embodiments, the methods can further comprise (c) introducing into the altered cell a polynucleotide encoding a site-directed nuclease (SDN), wherein the SDN binds to a target sequence present in the endogenous target polynucleotide (e.g., within the endogenous target polynucleotide). In some embodiments, the SDN cleaves within the endogenous target polynucleotide. In some embodiments, the method can further comprise (d) selecting a further altered cell.

In some embodiments, the methods described herein can comprise (a) introducing into the cell a polynucleotide encoding a site-directed nuclease (SDN), wherein the SDN binds to a target sequence present in the endogenous target polynucleotide, and (b) selecting an altered cell comprising the SDN. In some embodiments, the method can further comprise (c) introducing into the mitochondrion or the plastid a replacement nucleic acid comprising the internal polynucleotide. The method can further comprise (d) selecting a further altered cell comprising at least one altered mitochondrion genome or at least one altered plastid genome comprising the internal polynucleotide.

In some embodiments, the methods described herein can comprise (a) introducing into the mitochondrion or the plastid a replacement nucleic acid comprising the internal polynucleotide, and introducing into the nucleus a polynucleotide encoding a site-directed nuclease (SDN). For example, in some embodiments, the SDN and the replacement nucleic acid can be introduced concurrently. In some embodiments, the method can further comprise (b) selecting an altered cell comprising at least one altered mitochondrion genome or at least one altered plastid genome.

In some embodiments, the methods described herein can comprise introducing into a nucleus of the cell a third polynucleotide encoding an additional selectable marker polypeptide, wherein the additional selectable marker polypeptide provides tolerance to an additional selective agent. The method can further comprise selecting a cell that grows in the presence of the additional selective agent. In some embodiments, the cell is grown simultaneously in a presence of the additional selective agent and in a presence of a herbicide. In some embodiments, the cell is grown sequentially first in a presence of the additional selective agent and subsequently in a presence of the herbicide. In some embodiments, the cell is grown sequentially first in a presence of the herbicide and subsequently in a presence of the additional selective agent. The additional selective agent or the herbicide can be present at least at an effective concentration.

In some embodiments, the methods and compositions described herein can exhibit enhanced transformation efficiency. For example, in some embodiments, the methods and compositions described herein can enhance transformation efficiency by at least about 10%, at least about 20%, at least about 30%, at least about 40%, at least about 50%, at least about 60%, at least about 70%, at least about 80%, at least about 90%, at least about 100%, at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold as compared to that of a control method. In some embodiments, a control method can comprise genome modification or editing that lacks one or more compositions described herein (e.g., lacking the site-directed nuclease, the engineered site-directed nuclease) or one or more method steps (e.g., methods that do not comprise introducing the site-directed nuclease or the engineered site directed nuclease that binds to the target sequence present in the endogenous target polynucleotide).

In some embodiments, the altered cell or the further altered cell described herein can have at least about 50% of mitochondrial genomes of a plurality of mitochondrial genomes or at least about 50% of the plastid genomes of a plurality of plastid genomes in the cell (e.g., the altered cell or the further altered cell) comprise the replacement nucleic acid or a portion thereof (e.g., an internal polynucleotide). In some embodiments, the altered cell or the further altered cell described herein can have at least about 50% of mitochondrial genomes of a plurality of mitochondrial genomes or at least about 50% of the plastid genomes of a plurality of plastid genomes in the cell (e.g., the altered cell or the further altered cell) comprise the replacement nucleic acid or a portion thereof (e.g., an internal polynucleotide). In some embodiments, the altered cell or the further altered cell described herein can have at least about 60% of mitochondrial genomes of a plurality of mitochondrial genomes or at least about 60% of the plastid genomes of a plurality of plastid genomes in the cell (e.g., the altered cell or the further altered cell) comprise the replacement nucleic acid or a portion thereof (e.g., an internal polynucleotide). In some embodiments, the altered cell or the further altered cell described herein can have at least about 70% of mitochondrial genomes of a plurality of mitochondrial genomes or at least about 70% of the plastid genomes of a plurality of plastid genomes in the cell (e.g., the altered cell or the further altered cell) comprise the replacement nucleic acid or a portion thereof (e.g., an internal polynucleotide). In some embodiments, the altered cell or the further altered cell described herein can have at least about 80% of mitochondrial genomes of a plurality of mitochondrial genomes or at least about 80% of the plastid genomes of a plurality of plastid genomes in the cell (e.g., the altered cell or the further altered cell) comprise the replacement nucleic acid or a portion thereof (e.g., an internal polynucleotide). In some embodiments, the altered cell or the further altered cell described herein can have at least about 90% of mitochondrial genomes of a plurality of mitochondrial genomes or at least about 90% of the plastid genomes of a plurality of plastid genomes in the cell (e.g., the altered cell or the further altered cell) comprise the replacement nucleic acid or a portion thereof (e.g., an internal polynucleotide). In some embodiments, the altered cell or the further altered cell described herein can have at least about 95% of mitochondrial genomes of a plurality of mitochondrial genomes or at least about 95% of the plastid genomes of a plurality of plastid genomes in the cell (e.g., the altered cell or the further altered cell) comprise the replacement nucleic acid or a portion thereof (e.g., an internal polynucleotide). In some embodiments, the altered cell or the further altered cell described herein can achieve homoplasmy, where 100% of mitochondrial genomes of a plurality of mitochondrial genomes or 100% of the plastid genomes of a plurality of plastid genomes in the cell (e.g., the altered cell or the further altered cell) comprise the replacement nucleic acid or a portion thereof (e.g., an internal polynucleotide).

### Site-Directed Nucleases and Methods Utilizing the Site-Directed Nuclease

Provided herein are methods and compositions for replacement of an endogenous target sequence with a donor DNA or portion thereof (e.g., an internal polynucleotide described herein) or genome modification of a target sequence in a genome (e.g., a nucleus, a plastid, or a mitochondrial genome) of an organism or cell (e.g., a plant or plant cell).

In some embodiments, the methods and composition described herein can comprise replacing an endogenous target sequence with a replacement nucleic acid or a portion thereof (e.g., an internal polynucleotide). In some embodiments, the replacement of the endogenous target sequence with a replacement nucleic acid or a portion thereof (e.g., an internal polynucleotide) can be enhanced or increased by introducing a site-directed nuclease (SDN) that binds to a specific sequence (e.g., target sequence) within the endogenous target sequence to be replaced and cleaves the endogenous target sequence (e.g., single-strand or double-strand cleavage). As used herein, the term “within the endogenous target sequence” refers to a specific nucleotide region that resides entirely inside the defined boundaries of the endogenous target sequence to be replaced. A nuclease that targets and cleaves “within the endogenous target sequence” interacts specifically with a target sequence that is contained within the endogenous sequence, as opposed to sequences flanking or adjacent to the endogenous target sequence. For example, if the endogenous target sequence spans nucleotide positions N1 to N2 in the genome, “within the endogenous target sequence” encompasses binding and cleavage at any position between N1 and N2, inclusive. Cleavage “within the endogenous target sequence” may be distinct from cleavage at adjacent or flanking regions and may be specifically employed to disrupt, modify, or selectively target cells retaining the unmodified endogenous target sequence. For example, in some embodiments, the site-directed nuclease can cleave and degrade a mitochondrial DNA or a plastid DNA lacking the internal polynucleotide, thereby allowing selections of the altered cells or transformed cells comprising the internal polynucleotide. In some embodiments, the site-directed nuclease can be introduced before introducing the replacement nucleic acid (e.g., a replacement nucleic acid comprising an internal polynucleotide). In some embodiments, the site-directed nuclease can be introduced with the replacement nucleic acid (e.g., a replacement nucleic acid comprising an internal polynucleotide) concurrently, at the same time, or in the same solution. In some embodiments, the site-directed nuclease can be introduced after introducing the replacement nucleic acid (e.g., a replacement nucleic acid comprising an internal polynucleotide).

In some embodiments, site-directed nucleases can comprise restriction endonucleases that cleave DNA at specific sites without damaging bases. In some embodiments, the SDN is selected from the group consisting of: a polynucleotide guided nuclease, a transcription activator-like effector nuclease (TALEN), a zinc-finger nuclease, a meganuclease, a restriction enzyme, and any combination thereof. In some embodiments, restriction endonucleases can include Type I, Type II, Type III, and Type IV endonucleases, which can further include subtypes. In some embodiments, Type I and Type III systems, both a methylase and restriction activity can be contained in a single complex.

In some embodiments, site-directed nucleases can also include meganucleases, also known as homing endonucleases (HEases). In some embodiments, a meganuclease can bind and cut at a specific recognition site, which can be about 18 bp or more. In some embodiments, a meganuclease can be classified into four families based on conserved sequence motifs. In some embodiments, a meganuclease family can comprise LAGLIDADG (SEQ ID NO: 1), GIY-YIG, H—N—H, and His-Cys box families. In some embodiments, a meganuclease family can comprise of an amino acid sequence having at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% or more sequence identity to SEQ ID NO: 1. In some embodiments, motifs can participate in a coordination of metal ions and hydrolysis of phosphodiester bonds. In some embodiments, HEases can have long recognition sites and can tolerate sequence polymorphisms in their DNA substrates. In some embodiments, a naming convention for a meganuclease can be similar to a convention for other restriction endonuclease.

In some embodiments, a meganuclease can also be characterized by prefix F-, I-, or PI- for enzymes encoded by free-standing ORFs, introns, and inteins, respectively. In some embodiments, one step in a recombination process can involve polynucleotide cleavage at or near a recognition site. In some embodiments, a cleaving activity can be used to produce a double-strand break. In some embodiments, a recombinase can be from an Integrase or Resolvase family.

In some embodiments, compositions and methods described herein can use Transcription activator-like effector nucleases (TALENs; TAL effector nucleases). In some embodiments, TALENs can be a class of sequence-specific nucleases. In some embodiments, TALENs can be used to cleave (e.g., double-strand breaks, single-strand nicks) at or near specific target sequences (e.g., a TALEN DNA-binding site in a genome of a plant or other organism). In some embodiments, TALENs can be created by fusing a native or engineered transcription activator-like (TAL) effector DNA-binding domain, or functional part thereof, to a functional nuclease domain of an endonuclease. In some embodiments, the TALEN comprises a first polypeptide and a second polypeptide, wherein each of the first polypeptide and the second polypeptide comprise a nuclease domain. In some embodiments, the first polypeptide and the second polypeptide bind to target sequences that are separated by 12, 13, 14, 15, 16, 17, 18, 19, 20, 21, or 22 nucleotides. In some embodiments, the first polypeptide and the second polypeptide are capable of forming a dimer that has nuclease activity. In some embodiments, the TALEN, in dimeric form, cleaves at or near the target sequence(s). In some embodiments, the TALEN may be a single-chain TALEN that comprises a single-chain polypeptide having two copies of a nuclease domain. In some embodiments, the single-chain TALEN binds to the target sequence and cleaves at or near the target sequence.

In some embodiments, the functional nuclease domain for a TALEN can be derived from a TypeII restriction endonuclease. In some embodiments, the endonuclease can be a TypeIIS restriction endonuclease. In some embodiments, the endonuclease can have an amino-terminal DNA recognition domain and a carboxy-terminal nuclease domain. In some embodiments, the endonuclease can be FokI (SEQ ID NO: 2), and the functional nuclease domain can comprise SEQ ID NO: 3. In some embodiments, TALENs described herein can comprise a functional nuclease domain derived from FokI or a variant thereof. For example, in some embodiments, a variant of FokI can comprise or consist of an amino acid sequence that is at least about 10%, at least about 20%, at least about 30%, at least about 40%, at least about 50%, at least about 60%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more identical to an amino acid sequence of SEQ ID NO: 2. In some embodiments, TALENs described herein can comprise a functional nuclease domain or a variant derived from FokI (or a variant of FokI), wherein the functional nuclease domain or a variant thereof can comprise or consist of an amino acid sequence that is at least about 10%, at least about 20%, at least about 30%, at least about 40%, at least about 50%, at least about 60%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more identical to an amino acid sequence of SEQ ID NO: 3.

In some embodiments, the endonuclease can have an amino-terminal nuclease domain and a carboxy-terminal DNA-binding domain. Provided herein is an endonuclease comprising an amino-terminal nuclease domain. In some embodiments, the endonuclease can be NAP12 (SEQ ID NO: 4) or a variant thereof. In some embodiments, TALENs described herein can comprise a nuclease domain, a nuclease domain fragment, or a variant thereof derived from NAP12 (or derived from a variant of NAP12). In some embodiments, a variant of NAP12 can comprise or consist of an amino acid sequence that is at least about 10%, at least about 20%, at least about 30%, at least about 40%, at least about 50%, at least about 60%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more identical to an amino acid sequence of SEQ ID NO: 4. In some embodiments, TALENs described herein can comprise the nuclease domain (e.g., SEQ ID NO: 5), a nuclease domain fragment (SEQ ID NO: 60), or a variant thereof derived from NAP12 (or derived from a variant of NAP12). In some embodiments, TALENs described herein can comprise a functional nuclease domain, a functional nuclease domain fragment, or a functional variant thereof derived from NAP12 (or derived from a variant of NAP12). In some embodiments, the functional nuclease domain, the functional nuclease domain fragment, or the functional variant thereof can comprise or consist of an amino acid sequence that is at least about 10%, at least about 20%, at least about 30%, at least about 40%, at least about 50%, at least about 60%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more identical to an amino acid sequence of SEQ ID NO: 5 or SEQ ID NO: 60.

In some embodiments, TALENs comprising a nuclease domain (e.g., a nuclease domain fragment or a variant thereof) of NAP12 or variants of NAP12 can exhibit an enhanced nuclease activity, improve transformation efficiency (e.g., as measured by the % replacement of an endogenous target polynucleotide with an exogenous polynucleotide (e.g., a donor DNA or an internal polynucleotide described herein)) as compared to that of TALENs lacking the nuclease domain (e.g., the nuclease domain fragment or the variant thereof) of NAP12 or the variant of NAP12. In some embodiments, TALENs comprising a nuclease domain (e.g., a nuclease domain fragment or a variant thereof) of NAP12 or variants of NAP12 can exhibit an enhanced nuclease activity, wherein the nuclease activity is enhanced by at least about 10%, at least about 20%, at least about 30%, at least about 40%, at least about 50%, at least about 60%, at least about 70%, at least about 80%, at least about 90%, at least about 100%, at least about 1.5-fold, at least about 2-fold, at least about 3-fold, at least about 4-fold, at least about 5-fold or more when compared to that of TALENs lacking the nuclease domain (e.g., the nuclease domain fragment or the variant thereof) of NAP12 or the variant of NAP12. In some embodiments, TALENs comprising a nuclease domain (e.g., a nuclease domain fragment or a variant thereof) of NAP12 or variants of NAP12 can improve transformation efficiency (e.g., as measured by the % replacement of an endogenous target polynucleotide with an exogenous polynucleotide (e.g., a donor DNA or an internal polynucleotide described herein)), wherein the transformation efficiency is increased by at least about 10%, at least about 20%, at least about 30%, at least about 40%, at least about 50%, at least about 60%, at least about 70%, at least about 80%, at least about 90%, at least about 100%, at least about 1.5-fold, at least about 2-fold, at least about 3-fold, at least about 4-fold, at least about 5-fold or more when compared to that of TALENs lacking the nuclease domain (e.g., the nuclease domain fragment or the variant thereof) of NAP12 or the variant of NAP12. In some embodiments, TALENs comprising a nuclease domain (e.g., a nuclease domain fragment or a variant thereof) of NAP12 or variants of NAP12 can be further modified or engineered. In some embodiments, the engineered TALENs comprising a nuclease domain (e.g., a nuclease domain fragment or a variant thereof) of NAP12 or a variant of NAP12 can exhibit enhanced nuclease activity or improved transformation efficiency (e.g., as measured by % replacement of an endogenous target polynucleotide with an exogenous polynucleotide (e.g., donor DNA or an internal polynucleotide described herein)). In some embodiments, the engineered TALENs comprising a nuclease domain (e.g., a nuclease domain fragment or a variant thereof) of NAP12 or a variant of NAP12 can comprise a NAP12 nuclease domain associated to an amino-terminus of the transcription activator-like (TAL) domain (e.g., a DNA binding domain). In some embodiments, the engineered TALENs comprising a nuclease domain (e.g., a nuclease domain fragment or a variant thereof) of NAP12 or a variant of NAP12 can be associated to a carboxy-terminus of the TAL domain. In some embodiments, the engineered TALENs comprising a nuclease domain (e.g., a nuclease domain fragment or a variant thereof) of NAP12 or a variant of NAP12 can have at least about 2 NAP12 nuclease domains (or nuclease domain fragments), at least about 3 NAP12 nuclease domains, at least about 4 NAP12 nuclease domains, at least about 5 NAP12 nuclease domains, at least about 6 NAP12 nuclease domains, at least about 7 NAP12 nuclease domains, at least about 8 NAP12 nuclease domains, at least about 9 NAP12 nuclease domains, or at least about 10 NAP12 nuclease domains that are operably associated with the TAL domain. In some embodiments, the engineered TALENs comprising a nuclease domain (e.g., a nuclease domain fragment or a variant thereof) of NAP12 or a variant of NAP12 can comprise a plurality of NAP12 nuclease domains (e.g., comprising two NAP12 nuclease domains) such that the engineered TALENs exhibit enhanced dimerization between the plurality of NAP12 domains (e.g, between the two NAP12 nuclease domains).

In some embodiments, the engineered TALENs comprising a nuclease domain (e.g., a nuclease domain fragment or a variant thereof) of NAP12 or a variant of NAP12 can comprise modifications in the length of the DNA spacer region located between the two target sequences (e.g., DNA-binding sites) for the two polypeptides that comprise the TALEN-NAP12. For example, in some embodiments, the length of the DNA spacer region can be increased or decreased. In some embodiments, the first polypeptide and the second polypeptide that comprise the TALEN-NAP12 can bind to target sequences that are separated by at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

In some embodiments, the engineered TALENs comprising a nuclease domain (e.g., a nuclease domain fragment or a variant thereof) of NAP12 or a variant of NAP12 can comprise a modified linker between the TAL domain and NAP12 (or a variant thereof) nuclease domain. For example, in some embodiments, the length of the linker can be increased or decreased. In some embodiments, the linker can be increased or decrease by adding or removing at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, or at least 20 amino acids. In some embodiments, the linker can be a flexible linker. For example, in some embodiments, the linker can comprise glycine-serine repeats, polyglycine (PolyGly), or PEG-based linkers. In some embodiments, the linker can comprise can comprise or consist of an amino acid sequence that is at least about 10%, at least about 20%, at least about 30%, at least about 40%, at least about 50%, at least about 60%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more identical to an amino acid sequence of SEQ ID NO: 61. In some embodiments, the linker can comprise or consist of SEQ ID NO: 61. In some embodiments, the engineered TALENs comprising a nuclease domain (e.g., a nuclease domain fragment or a variant thereof) of NAP12 or a variant of NAP12 described herein can exhibit an enhanced nuclease activity, improve transformation efficiency (e.g., as measured by the % replacement of an endogenous target polynucleotide with an exogenous polynucleotide (e.g., a donor DNA or an internal polynucleotide described herein)) as compared to that of the native TALENs (e.g., without one or more modifications described herein). As used herein, the term “native TALENs” refers to TALENs that have not been modified or optimized in their DNA-binding domains, nuclease domains, spacer regions, linker regions, or other structural components as described herein. In some embodiments, the engineered TALENs comprising a nuclease domain (e.g., a nuclease domain fragment or a variant thereof) of NAP12 or a variant of NAP12 can exhibit an enhanced nuclease activity, wherein the nuclease activity is enhanced by at least about 10%, at least about 20%, at least about 30%, at least about 40%, at least about 50%, at least about 60%, at least about 70%, at least about 80%, at least about 90%, at least about 100%, at least about 1.5-fold, at least about 2-fold, at least about 3-fold, at least about 4-fold, at least about 5-fold or more when compared to that of native TALENs. In some embodiments, the engineered TALENs comprising a nuclease domain (e.g., a nuclease domain fragment or a variant thereof) of NAP12 or a variant of NAP12 can improve transformation efficiency (e.g., as measured by the % replacement of an endogenous target polynucleotide with an exogenous polynucleotide (e.g., a donor DNA or an internal polynucleotide described herein)), wherein the transformation efficiency is increased by at least about 10%, at least about 20%, at least about 30%, at least about 40%, at least about 50%, at least about 60%, at least about 70%, at least about 80%, at least about 90%, at least about 100%, at least about 1.5-fold, at least about 2-fold, at least about 3-fold, at least about 4-fold, at least about 5-fold or more when compared to that the native TALENS.

In some embodiments, a dimerization of the nuclease domain may be required for cleavage activity. In some embodiments, two TALENs with nuclease domains capable of dimerization may have DNA-binding sites separated by at least about 5, 6, 7, 8, 9, 10, 11, 12, 13, 14, 15, 16, 17, 18, 19, 20, 21, or 22 base pairs. In some embodiments, the TALENs can be a single-chain TALEN (scTALEN), in which two functional nuclease domains are fused to a single TAL effector DNA-binding domain. In some embodiments, a scTALEN may have an amino-terminal TAL effector DNA-binding domain and two carboxy-terminal nuclease domains. In some embodiments, a scTALEN may have two amino-terminal nuclease domains and a carboxy-terminal TAL effector DNA-binding domain. In some embodiments, a unique, modular TAL effector DNA binding domain can allow for a design of proteins with potentially any given DNA recognition specificity.

Disclosed herein in some embodiments, are compositions and methods comprising use of zinc finger nucleases (ZFNs). In some embodiments, ZFNs can be engineered cleavage (e.g., double-strand break, single-strand nick) inducing agents comprised of a zinc finger DNA binding domain and a double-strand-break-inducing agent domain. In some embodiments, recognition site specificity can be conferred by a zinc finger domain, which can comprise two, three, or four zinc fingers, for example having a C2H2 structure. In some embodiments, a zinc finger domain can be amenable for designing polypeptides which specifically bind a selected polynucleotide recognition sequence. In some embodiments, a ZFN can consist of an engineered DNA-binding zinc finger domain linked to a non-specific endonuclease domain, for example, a nuclease domain from a Type IIS endonuclease such as FokI (SEQ ID NO: 2) or NAP12 (SEQ ID NO: 4) or an endonuclease having at least 80%, 90%, or 95% sequence identity to SEQ NOs: 2 or 4. In some embodiments, additional functionalities can be fused to a zinc-finger binding domain, including transcriptional activator domains, transcription repressor domains, and methylases. In some examples, a dimerization of the nuclease domain may be required for cleavage activity. In some embodiments, each zinc finger can recognize, for example, three consecutive base pairs in a target DNA. In some embodiments, a 3-finger domain can recognize a sequence of 9 contiguous nucleotides, with a dimerization requirement of a nuclease, two sets of zinc finger triplets can be used to bind an 18 nucleotide recognition sequence. In some embodiments, the ZFN can be a single-chain ZFN (scZFN), in which two functional nuclease domains are fused to a single zinc-finger binding domain. In some embodiments, a scZFN may have an amino-terminal zinc-finger binding domain and two carboxy-terminal nuclease domains. In some embodiments, a scZFN may have two amino-terminal nuclease domains and a carboxy-terminal zinc-finger binding domain.

In some embodiments, a site-directed nuclease disclosed herein can be a polynucleotide guided nuclease, e.g., a polynucleotide guided polypeptide with nuclease activity (e.g., a CRISPR nuclease). In some embodiments, a polynucleotide guided nuclease can be a Cas protein of a CRISPR/Cas system, a modified version thereof, or a biologically active fragment thereof. In some embodiments, a polynucleotide guided nuclease can be a Cas9 protein, a modified version thereof, or a biologically active fragment thereof. In some embodiments, a polynucleotide guided nuclease can be a Cas3 protein, a modified version thereof, or a biologically active fragment thereof. In some embodiments, a polynucleotide guided nuclease can be a Cpf1 protein, a modified version thereof, or a biologically active fragment thereof. In some embodiments, a polynucleotide guided nuclease can be a CRISPR nuclease, a modified version thereof, or a biologically active fragment thereof. In some embodiments, a polynucleotide guided nuclease can be a nuclease domain of a Cas protein, a modified version thereof, or a biologically active fragment thereof. In some embodiments, a polynucleotide guided nuclease can be a MAD protein, a modified version thereof, or a biologically active fragment thereof. In some embodiments, a polynucleotide guided nuclease can be an Argonaute protein, a modified version thereof, or a biologically active fragment thereof. In some embodiments, a polynucleotide guided nuclease can be a MAD2 protein, a modified version thereof, or a biologically active fragment thereof. In some embodiments, a polynucleotide guided nuclease can be a MAD7 protein, a modified version thereof, or a biologically active fragment thereof. In some embodiments, a polynucleotide guided nuclease can form a complex with a guide polynucleotide. In some embodiments, a polynucleotide guided nuclease can be directed to a target nucleic acid by a guide polynucleotide. In some embodiments, a polynucleotide guided nuclease can complex with a guide polynucleotide to recognize a target nucleic acid. In some embodiments, a polynucleotide guided nuclease can introduce a single strand or double strand break at a specific target site (e.g., the genome of a cell).

In some embodiments, as used herein, the term “guide polynucleotide”, can refer to a polynucleotide sequence that can form a complex with a polynucleotide guided nuclease (e.g., a Cas protein, a MAD protein). In some embodiments, a guide polynucleotide can direct a polynucleotide guided nuclease to recognize and cleave (or nick) a DNA target site. In some embodiments, the terms “guide polynucleotide” and “guide polynucleic acid” can be used interchangeably herein. In some embodiments, a guide polynucleotide can be comprised of a single molecule (unimolecular) or two molecules (bimolecular). In some embodiments, a type II CRISPR/Cas system from bacteria can employ a crRNA and tracrRNA to guide a Cas polypeptide to a nucleic acid target. In some embodiments, a crRNA (CRISPR RNA) can contain a region complementary to one strand of a double strand DNA target. In some embodiments, a crRNA can base pair with a tracrRNA (trans-activating CRISPR RNA) to form an RNA duplex that can direct a Cas polypeptide to recognize and optionally cleave a DNA target. In some embodiments, a guide polynucleotide sequence can be an RNA sequence, a DNA sequence, or a combination thereof (an RNA-DNA combination sequence). In some embodiments, a guide polynucleotide that solely can comprise ribonucleic acids can also be referred to as a “guide RNA” (gRNA). In some embodiments, the term “single guide RNA” (sgRNA) can refer to a synthetic fusion of two RNA molecules, for example, a crRNA (CRISPR RNA) comprising a variable targeting domain (or VT domain), and a tracrRNA. In some embodiments, a guide RNA can comprise a VT domain of 12 to 30 nucleotide sequences and an RNA fragment that can interact with a Cas protein. In some embodiments, a guide polynucleic acid can be introduced into a plant cell via transformation of a recombinant DNA construct comprising a polynucleotide encoding a guide polynucleic acid operably linked to a promoter functional in a plant; e.g., a plant U6 polymerase III promoter, a CaMV 35S polymerase II promoter, a mitochondrial promoter, a plastid promoter. In some embodiments, a plurality of guide polynucleic acids can be multiplexed to target multiple target nucleic acids. For example, 2, 3, 4, 5, 6, 7, 8, 9, 10, or more than 10 target nucleic acids can be targeted simultaneously or iteratively.

In some embodiments, a Cas protein can comprise a Cas9 protein. In some embodiments, a Cas9 protein can comprise a Cas9 sequence listed in SEQ ID NOS: 462, 474, 489, 494, 499, 505, or 518 of WO2007/025097 and incorporated herein by reference. In some embodiments, a Cas9 protein can comprise or consist of a nucleic acid sequence having at least about 10%, at least about 20%, at least about 30%, at least about 40%, at least about 50%, at least about 60%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more sequence identity to SEQ ID NOS: 462, 474, 489, 494, 499, 505, or 518 of WO2007/025097 and incorporated herein by reference. In some embodiments, a Cas9 protein can be any one of Cas9 proteins disclosed in WO2007/025097. In some embodiments, a Cas9 protein can be a Streptococcus pyogenes Cas9 protein.

In some embodiments, a Cas9 protein can unwind a DNA duplex in close proximity to a genomic target site. In some embodiments, a Cas9 protein can cleave both DNA strands upon recognition of a target sequence by a guide polynucleic acid. In some embodiments, a Cas9 endonuclease can cleave only if a correct protospacer-adjacent motif (PAM) is approximately oriented at a 3′ end of a target sequence. In some embodiments, a mutagenesis of Streptococcus pyogenes Cas9 catalytic domains can produce “nicking” enzymes (Cas9n) that can induce single-strand nicks rather than double-strand breaks.

In some embodiments, a polynucleotide guided nuclease can be a MAD polypeptide, e.g., a MAD2 or a MAD7 polypeptide, with amino acid sequence corresponding to SEQ ID NO: 2 and SEQ ID NO: 7 of U.S. Pat. No. 9,982,279, respectively (herein incorporated by reference). In some embodiments, a polynucleotide guided nuclease can be a MAD polypeptide, e.g., a MAD2 or a MAD7 polypeptide, having least about 10%, at least about 20%, at least about 30%, at least about 40%, at least about 50%, at least about 60%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more sequence identity to SEQ ID NO: 2 or SEQ ID NO: 7 of U.S. Pat. No. 9,982,279, respectively (herein incorporated by reference). In some embodiments, the MAD7 polypeptide can comprise a sequence having at least 80%, 90%, or 95% sequence identity to SEQ ID NO: 31.

In some embodiments, a MAD7 can be a Class 2 Type V-A CRISPR-Cas system isolated from Eubacterium rectale and re-engineered by INSCRIPTA™ (Boulder, CO). In some embodiments, analogous to Cas9, MAD7 can be an RNA-guided nuclease with a diverse protein structure, mechanism of action, and a demonstrated gene editing activity in E. coli and yeast cells. In some embodiments, similar to Acidaminococcus sp. Cas12a, MAD7 does not require a tracrRNA and prefers T-rich PAMs (TTTV and CTTV). In some embodiments, a mutagenesis of MAD2 or MAD7 can produce a “nicking” enzyme that can induce single-strand nicks rather than double-strand breaks.

In some embodiments, a polynucleotide guided nuclease may be an Argonaute protein such as Natronobacterium gregoryi Argonaute (“NgAgo”). In some embodiments, an Argonaute protein can be a DNA-guided endonuclease. In some embodiments, an Argonaute protein can bind a guide DNA such as a 5′-phosphorylated single-stranded guide DNA (gDNA) of, for example, 24 nucleotides. In some embodiments, an Argonaute protein can create a site-specific target nucleic acid (e.g., DNA) break (e.g., double-stranded breaks) when loaded with a gDNA. In some embodiments, an Argonaute protein/gDNA system may not require a protospacer-adjacent motif (PAM) for recognition of a target nucleic acid.

In some embodiments, a site-directed nuclease as used herein can be a wildtype or a modified form of a site-directed nuclease. In some embodiments, a site-directed nuclease can be an active variant, an inactive variant, or a fragment of a wild type or modified site-directed nuclease. In some embodiments, a site-directed nuclease can comprise an amino acid change such as a deletion, replacement, insertion, substitution, variant, mutation, fusion, chimera, or any combination thereof relative to a wild-type version of a site-directed nuclease. In some embodiments, a site-directed nuclease can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary site-directed nuclease (e.g., Cas9 from S. pyogenes). In some embodiments, a site-directed nuclease can be a polypeptide with at most about 5%, 10%, 20%, 30%, 40%, 50%, 60%, 70%, 80%, 90%, 100% sequence identity and/or sequence similarity to a wild type exemplary site-directed nuclease. In some embodiments, variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified site-directed nuclease or a portion thereof.

In some embodiments, a nucleic acid encoding a site-directed nuclease can be codon optimized for efficient translation into protein in a particular cell, organelle (e.g., nucleus, plastid, or mitochondrion), or organism (e.g., wheat or rice).

In some embodiments, a nucleic acid encoding a site-directed nuclease can be stably integrated in a genome (nuclear, mitochondrial, plastid) of a cell. In some embodiments, a nucleic acid encoding a site-directed nuclease can be operably linked to a regulatory sequence active in a cell. In some embodiments, a nucleic acid encoding a site-directed nuclease can be in an expression construct. In some embodiments, an expression construct can include any regulatory sequence that can direct expression of a nucleic acid sequence of interest (promoter, terminator, RNA-editing site). In some embodiments, an expression construct can include any nucleic acid sequence that encodes a peptide capable of targeting a protein into an organelle of interest (e.g., into a nucleus, mitochondrion, or plastid).

In some embodiments, a site-directed nuclease coding sequence can be modified to use codons preferred by a target organism, e.g., a plant, maize, or soybean (nuclear, mitochondrial or plastid) codon-optimized sequence. In some embodiments, a sequence that encodes a site-directed nuclease can be operably linked to one or more sequences encoding nuclear localization signals; e.g., to a SV40 nuclear targeting signal upstream of a site-directed nuclease coding region and a bipartite VirD2 nuclear localization signal downstream of the site-directed nuclease coding region. In some embodiments, a sequence that encodes a site-directed nuclease can be operably linked to one or more sequences encoding chloroplast or mitochondrial localization signals, i.e., a chloroplast transit sequence or a mitochondrial targeting sequence.

In some embodiments, a site-directed nuclease can be provided in any form. In some embodiments, a site-directed nuclease can be provided in a form of a protein, such as a site-directed nuclease alone or in the case of a polynucleotide guided nuclease complexed with a guide nucleic acid. In some embodiments, a site-directed nuclease can be provided in a form of a nucleic acid encoding a site-directed nuclease, such as an RNA (e.g., messenger RNA (mRNA)) or DNA.

In some embodiments, a site-directed nuclease described herein can be further modified. In some embodiments, a site-directed nuclease can comprise an organelle targeting amino acid sequence (OTS). For example, in some embodiments, the OTS can be amino acid sequences that directs the protein to specific subcellular organelles. In some embodiments, the OTS can be a mitochondrial targeting amino acid sequence (MTS). In some embodiments, the OTS can be a plastid targeting amino acid sequence (PTS). In some embodiments, the OTS can be a chloroplast targeting amino acid sequence (CTS). In some embodiments, the terms “PTS” and “CTS” are used interchangeably. In some embodiments, the MTS can comprise an amino acid sequence derived from a yeast, an alga, a plant, or an animal. In some embodiments, the MTS may comprise an amino acid sequence derived from Saccharomyces cerevisiae, Chlamydomonas reinhardtii, rice, wheat, corn, or soybean. In some embodiments, the PTS may comprise an amino acid sequence derived from an alga or a plant. In some embodiments, the PTS may comprise an amino acid sequence derived from Chlamydomonas reinhardtii, rice, wheat, corn, or soybean.

In some embodiments, MTS or PTS can be at least about 5 amino acids, at least about 10 amino acids, at least about 15 amino acids, at least about 20 amino acids, at least about 25 amino acids, at least about 30 amino acids, at least about 35 amino acids, at least about 40 amino acids, at least about 45 amino acids, at least about 50 amino acids, at least about 55 amino acids, at least about 60 amino acids, or more long.

In some embodiments, the MTS can comprise or consist of an amino acid sequence that is at least about 60%, at least about 65%, at least about 70%, at least about 75%, at least about 80%, at least about 85%, at least about 90%, at least about 91%, at least about 92%, at least about 92%, at least about 94%, at least about 95%, at least about 96%, at least about 97%, at least about 98%, at least about 99%, or more identical to an amino acid sequence of SEQ ID NO: 36 or SEQ ID NO: 42 or SEQ ID NO: 129. In some embodiments, the MTS can comprise or consist of an amino acid sequence of SEQ ID NO: 36 or SEQ ID NO: 42 or SEQ ID NO: 129.

In some embodiments, the PTS can comprise or consist of an amino acid sequence that is at least about 60%, at least about 65%, at least about 70%, at least about 75%, at least about 80%, at least about 85%, at least about 90%, at least about 91%, at least about 92%, at least about 92%, at least about 94%, at least about 95%, at least about 96%, at least about 97%, at least about 98%, at least about 99%, or more identical to an amino acid sequence of SEQ ID NO: 88. In some embodiments, the PTS can comprise or consist of an amino acid sequence of SEQ ID NO: 88.

In some embodiments, the methods and compositions described herein can comprise introducing a plurality of SDNs into a cell (e.g., into the nucleus of a cell). In some embodiments, the plurality of SDNs can comprise at least two SDNs, at least three SDNs, at least four SDNs, at least four SDNs, at least five SDNs, at least six SDNs, at least seven SDNs, at least eight SDNs, at least nine SDNs, or at least ten SDNs. In some embodiments, a plurality of SDNs can bind to the same target sequence within the endogenous target polynucleotide. In some embodiments, a plurality of SDNs can bind to two or more different target sequences within the endogenous target polynucleotide. In some embodiments, a plurality of SDNs may comprise identical SDN (e.g., multiple TALENs). In some embodiments, a plurality of SDNs may comprise distinct SDN (e.g., TALENs and Cas Protein). For example, in some embodiments, a plurality of SDNs can be from the group consisting of: a polynucleotide guided nuclease, a transcription activator-like effector nuclease (TALEN), a zinc-finger nuclease, a meganuclease, a restriction enzyme, and any combination thereof.

In some embodiments, a coding sequence for a site-directed nuclease can be modified to use codons preferred by a target organism, e.g., a plant, maize, or soybean (nuclear, mitochondrial or plastid) codon-optimized sequence. In some embodiments, a sequence that encodes a site-directed nuclease can be operably linked to one or more sequences encoding nuclear localization signals; e.g., to a SV40 nuclear targeting signal upstream of a site-directed nuclease coding region and a bipartite VirD2 nuclear localization signal downstream of the site-directed nuclease coding region. In some embodiments, a sequence that encodes a site-directed nuclease can be operably linked to one or more sequences encoding chloroplast or mitochondrial localization signals, i.e., a chloroplast transit sequence or a mitochondrial targeting sequence.

### Target Sites for Genome Modification

In some embodiments, the terms “target site”, “target sequence”, “target polynucleotide”, “target polynucleic acid”, “target locus”, “genomic target site”, “genomic target sequence”, and “genomic target locus” can be used interchangeably herein. In some embodiments, an “endogenous target polynucleotide” can refer to an endogenous polynucleotide that contains a target site. In some embodiments, a target polynucleic acid can refer to a polynucleotide sequence in a genome (e.g., a plastid or a mitochondrial genome). In some embodiments, a genome can be part of a plant cell. In some embodiments, a target polynucleic acid can refer to a site (e.g., a DNA-binding site in a genome) recognized by a site-directed nuclease. In some embodiments, a target polynucleic acid can refer to a site (e.g., in a genome) at (or near to) which a single-strand or double-strand break can be induced. In some embodiments, a target site can be an endogenous site in a genome. In some embodiments, a target site can be found in a heterologous genomic location compared to where it occurs in nature. In some embodiments, as used herein, the terms “endogenous target sequence” and “native target sequence” can be used interchangeably herein and can refer to a target sequence that can be endogenous or native to a genome of an organism. In some embodiments, endogenous target sequence can occur at an endogenous or native position of a target sequence in a genome of an organism. In some embodiments, the target site is not present in the replacement nucleic acid.

In some embodiments, a target polynucleic acid can be DNA, RNA, or both. In some embodiments, a target polynucleic acid can be DNA (e.g., target DNA). In some embodiments, a target polynucleic acid can be genomic DNA. In some embodiments, a target polynucleic acid can be nuclear DNA, mitochondrial DNA, plastid DNA, or any combination thereof.

In some embodiments, the terms “artificial target site” and “artificial target sequence” can be used interchangeably herein and can refer to a target sequence that has been introduced into a genome of a plant. In some embodiments, such an artificial target sequence can be identical in sequence to an endogenous or native target sequence in a genome of an organism but may be located in a different position (i.e., a non-endogenous or non-native position) in a genome of an organism.

In some embodiments, an “altered target site”, “altered target sequence”, “modified target site”, “modified target sequence” can be used interchangeably herein and can refer to a target sequence as disclosed herein that can comprise at least one alteration when compared to a non-altered target sequence. In some embodiments, such “alterations” can include, for example: (i) a substitution of at least one nucleotide, (ii) a deletion of at least one nucleotide, (iii) an insertion of at least one nucleotide, or (iv) any combination of (i)-(iii).

In some embodiments, a length of a target site can vary and can include, for example, target sites that are at least 12, 13, 14, 15, 16, 17, 18, 19, 20, 21, 22, 23, 24, 25, 26, 27, 28, 29, 30 or more nucleotides in length. In some embodiments, a target site can be palindromic. In some embodiments, a palindromic sequence can comprise a sequence that on one strand reads the same in the opposite direction on the complementary strand. In some embodiments, a nick/cleavage site can be within a target sequence. In some embodiments, a nick/cleavage site can be outside of a target sequence. In some embodiments, a cleavage could occur at nucleotide positions immediately opposite each other to produce a blunt end cut or, in other cases, incisions could be staggered to produce single-stranded overhangs, also called “sticky ends”, which can be either 5′ overhangs, or 3′ overhangs.

In some embodiments, active variants of genomic target sites can also be used. In some embodiments, active variants can comprise at least 65%, 70%, 75%, 80%, 85%, 90%, 91%, 92%, 93%, 94%, 95%, 96%, 97%, 98%, 99% or more sequence identity to a given target site. In some embodiments, active variants can retain biological activity. In some embodiments, active variants can be recognized by a site-directed nuclease. In some embodiments, active variants can be cleaved (e.g., either single-strand nicks or double-strand breaks) by a site-directed nuclease.

### Methods Utilizing a Site-Directed Nuclease

In some embodiments, a polynucleotide encoding a site-directed nuclease described herein can be stably integrated into an organellar genome of a cell (a nuclear, mitochondrial, or plastid genome) or can be transiently expressed in an organellar genome of a cell. In some embodiments, a polynucleotide encoding a site-directed nuclease described herein can be stably integrated into a nuclear genome of the cell.

In some embodiments, a polynucleotide can encode a modified site-directed nuclease comprising an enzymatically active site-directed nuclease (e.g., TALEN, meganuclease, restriction enzyme, Cas polypeptide, MAD polypeptide) fused to an organellar transport sequence (e.g., a mitochondrial targeting peptide (e.g., a mitochondrion targeting sequence, MTS) or a plastid targeting peptide (e.g., a plastid targeting sequence, PTS)). In some embodiments, a polynucleotide encoding a modified site-directed nuclease described herein can be stably integrated into a nuclear genome of a cell or can be transiently expressed in a nuclear genome of a cell.

In some embodiments, expression of a polynucleotide encoding a site-directed nuclease or a modified site-directed nuclease can be under control of a promoter. In some embodiments, a promoter can be a constitutive promoter, a tissue-specific promoter, or an inducible promoter, e.g., a temperature-inducible, stress-inducible, developmental stage inducible, or chemically inducible promoter. In some cases, expression of a site-directed nuclease or a modified site-directed nuclease in a cell line or transgenic organism may have little or no consequence to cell viability.

In some embodiments, a polynucleotide encoding a site-directed nuclease or a modified site-directed nuclease can be introduced into a cell via direct delivery methods, biological delivery methods, or a combination thereof. In some embodiments, a direct delivery method can comprise a particle bombardment, an electroporation, a vacuum infiltration, a nanoparticle-mediated delivery, or any combination thereof. In some embodiments, a biological delivery method can comprise an Agrobacterium-mediated DNA delivery method.

In some embodiments, an Agrobacterium-mediated procedure for transformation of Japonica rice callus tissue can be performed essentially as described in the scientific literature (Hiei, Y., Komari, T. 2008 Agrobacterium-mediated transformation of rice using immature embryos or calli induced from mature seed. Nature Protocols 3, 824-834).

In some embodiments, Agrobacterium strain AGL-1 harboring a binary vector, e.g., a pCAMBIA0380-derived vector, can be cultured on 1.5% agar plates containing LB medium, 15 mg/L rifampicin, 50 mg/L carbenicillin, 50 mg/L kanamycin for 3-4 days in the dark at ambient room temperature or 28° C. (Spring/Summer at room temperature; Autumn/Winter in a 28° C. incubator). Agrobacterium culture can be scraped from the plates and suspended in AAM medium to yield an OD₆₆₀ of approximately 0.1. The OD₆₆₀ value can be varied, e.g., from 0.005 to 0.3. The calli can then be soaked in 5 ml sterile water in a 90-mm Petri dish. The Petri dish can then be slanted, the calli can be collected at the lower side of the Petri dish, and the water can then be removed. 1.0 ml of bacterial suspension can be added to the slanted dish by Pasteur pipette and the calli and the bacterial suspension can be mixed by pipetting. The slanted dish can then be incubated for 2 min at 25° C. The calli and the bacterial suspension can be mixed again by pipetting. The bacterial suspension can be removed by Pasteur pipette. The calli can then be blotted dry with a sterilized filter paper to remove excess bacteria. The calli can then be transferred with forceps onto a sterilized filter paper that has been moistened with 1 ml of AAM medium placed on 2N6-AS medium and the plate can be sealed with Parafilm. The plate can then be incubated in the dark at 25° C. for 3 days. After 3 days of co-cultivation at 25° C. in the dark, the calli can be washed four times in sterilized water and then washed two times in sterilized water containing 250 mg/L Cefataxime (or 250 mg/L Timentin) to remove Agrobacterium.

In some embodiments, a site-directed nuclease may be a polynucleotide guided nuclease (e.g., Cas polypeptide, MAD polypeptide), wherein corresponding guide polynucleotides (e.g., guide RNAs, single guide RNAs, or crRNAs) can be introduced by a variety of methods into cells containing a stably-integrated and expressed expression cassette for a polynucleotide guided nuclease. In some embodiments, a guide polynucleotide (e.g., guide RNAs, single guide RNAs, or crRNAs) can be chemically or enzymatically synthesized and introduced into a polynucleotide guided polypeptide expressing cells via direct delivery methods such a particle bombardment or electroporation. In some embodiments, a guide polynucleic acid can be fused to an RNA molecule that allows for transport into an organelle. In some embodiments, a guide polynucleotide can be fused to an RNA molecule that allows for binding to a protein that facilitates transport into an organelle. In some embodiments, a guide polynucleotide can be transported into an organelle by association with a modified polynucleotide guided nuclease comprising an enzymatically active polynucleotide guided nuclease fused to an organellar transport sequence.

In some embodiments, a gene can efficiently express a guide polynucleotide in a target cell. In some embodiments a guide polynucleotide can comprise a guide RNA, a single guide RNA, a crRNA, or a combination thereof. In some embodiments a gene that can efficiently express a guide polynucleotide in a target cell can be synthesized chemically, enzymatically or in a biological system. In some embodiments, a gene that can efficiently express a guide polynucleotide in a target cell can be introduced into a polynucleotide guided polypeptide expressing cell, via direct delivery methods, biological delivery methods, or a combination thereof. In some embodiments, a direct delivery method can comprise a particle bombardment, an electroporation, a vacuum infiltration, a nanoparticle-mediated delivery, or any combination thereof. In some embodiments, a biological delivery method can comprise an Agrobacterium-mediated DNA delivery method.

### Methods for Selecting a Plant Comprising an Altered Organellar Genome

Disclosed herein in some embodiments, are methods for selecting a plant comprising an altered organellar genome (e.g., mitochondrial or plastid genome). In some embodiments, a method can be used to identify those cells having an altered genome at or near a target site without using a screenable or selectable marker phenotype. In some embodiments, a method can comprise directly analyzing a target sequence to detect any change in a target sequence, including but not limited to PCR methods, sequencing methods, nuclease digestion, Southern blots, and any combination thereof.

In some embodiments, sufficient homology or sequence identity can indicate that two polynucleotide sequences can have sufficient structural similarity to act as substrates for a homologous recombination reaction. In some embodiments, a structural similarity can include an overall length of each polynucleotide fragment, a sequence similarity of each polynucleotide, or a combination thereof. In some embodiments, a sequence similarity can be described by a percent sequence identity over a whole length of multiple sequences, by conserved regions comprising localized similarities such as contiguous nucleotides having 100% sequence identity, by percent sequence identity over a portion of a length of multiple sequences, or any combination thereof.

In some embodiments, an amount of homology or sequence identity shared by a target and a donor polynucleotide can vary. For example, a length of sequence homology can be at least about 20 bp, at least about 50 bp, at least about 75 bp, at least about 100 bp, at least about 150 bp, at least about 250 bp, at least about 300 bp, at least about 400 bp, at least about 500 bp, at least about 600 bp, at least about 700 bp, at least about 800 bp, at least about 900 bp, at least about 1000 bp, at least about 1250 bp, at least about 1500 bp, at least about 1750 bp, at least about 2000 bp, at least about 2.5 kb, at least about 3 kb, at least about 4 kb, at least about 5 kb, at least about 6 kb, at least about 7 kb, at least about 8 kb, at least about 9 kb, or at least about 10 kb. In some embodiments, an amount of homology can also be described by a percent sequence identity over a fully aligned length of two polynucleotides which can include a percent sequence identity of at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, sufficient homology can include any combination of polynucleotide length, global percent sequence identity, conserved regions of contiguous nucleotides, local percent sequence identity, or any combination thereof. In some embodiments, a sufficient homology can be described as a region of 75-150 bp having at least 80% sequence identity to a region of a target locus. In some embodiments, a sufficient homology can also be described by a predicted ability of two polynucleotides to specifically hybridize under high stringency conditions.

In some embodiments, a plant cell having an introduced sequence can be grown or regenerated into a plant. In some embodiments, a plant can then be grown, and either pollinated with a same transformed strain or with a different transformed or untransformed strain, and a resulting progeny having a desired characteristic and/or comprising an introduced polynucleotide or polypeptide identified. In some embodiments, two or more generations can be grown to ensure that a polynucleotide can be stably maintained and inherited, and seeds harvested.

In some embodiments, any plant can be used. In some embodiments, a plant can comprise a monocot plant or a dicot plant. In some embodiments, a monocot plant can comprise a maize (Zea mays), a rice (Oryza sativa), a rye (Secale cereale), a sorghum (Sorghum bicolor, Sorghum vulgare), a millet (e.g., pearl millet (Pennisetum glaucum)), a proso millet (Panicum miliaceum), a foxtail millet (Setaria italica), a finger millet (Eleusine coracana), a wheat (Triticum aestivum), a sugarcane (Saccharum spp.), an oat (Avena), a barley (Hordeum), a switchgrass (Panicum virgatum), a pineapple (Ananas comosus), a banana (Musa spp.), a palm, an ornamental, a turfgrass, another grass plant, or any combination thereof. In some embodiments, a dicot plant can comprise a soybean (Glycine max), a canola (Brassica napus and B. campestris), an alfalfa (Medicago sativa), a tobacco (Nicotiana tabacum), an Arabidopsis (Arabidopsis thaliana), a sunflower (Helianthus annuus), a cotton (Gossypium arboreum), a peanut (Arachis hypogaea), a tomato (Solanum lycopersicum), a potato (Solanum tuberosum) plant, or any combination thereof.

In some embodiments, after creating a designed change in an organellar DNA, a next step can be to maintain an edited organellar DNA in a pool of unmodified organellar DNA and to shift a balance among organellar DNA to favor a maintenance of genome edited organellar DNA. In some embodiments, this can be achieved by reducing an amplification of unmodified organellar DNA. In some embodiments, site-directed nucleases can be designed for multiple target sites in an unmodified organelle genome. In some embodiments, a donor polynucleotide (e.g., a replacement nucleic acid) can comprise an internal polynucleotide. In some embodiments, an internal polynucleotide can comprise a sequence heterologous to the genome of the mitochondrion or the plastid. In some embodiments, a donor polynucleotide (e.g., a replacement nucleic acid comprising an internal polynucleotide) can be designed such that a target site has been altered to no longer be recognized by a relevant site-directed nuclease. In some embodiments, an expression of a site-directed nuclease can result in an introduction of single-strand or double-strand breaks into an unmodified organellar DNA and can thereby increase a proportion of modified genomes. In some embodiments, a cell can be pretreated with relevant site-directed nucleases to introduce cleavages in organellar DNA. In some embodiments, a pretreatment can reduce a number of organelle DNA molecules available for homologous recombination. In some embodiments, the pretreatment can reduce a number of organelle DNA molecules available for homologous recombination by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% as compared to a number of organelle DNA molecules of untreated cell (e.g., cells received no treatment with one or more nucleases described herein).

In some embodiments, a cell may be selected that is homoplasmic for an altered genome of an organelle (e.g., a mitochondrion or a plastid). In some embodiments, a cell may be selected that comprises a plurality of mitochondrial (or plastid) genomes, wherein at least 10%-100% of the plurality of mitochondrial (or plastid) genomes comprise the edited mitochondrial (or plastid) genome. In some embodiments, the selected cell may comprise a plurality of mitochondrial (or plastid) genomes wherein about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100% of the plurality of mitochondrial (or plastid) genomes comprise the edited mitochondrial (or plastid) genome. In some embodiments, the selected cell may comprise a plurality of mitochondrial (or plastid) genomes wherein about 10%, about 20%, about 30%, about 40%, about 50%, about 60%, about 70%, about 80%, about 90%, or about 100% of the plurality of mitochondrial (or plastid) genomes comprise the edited mitochondrial (or plastid) genome. In some embodiments, the selected cell may comprise a plurality of mitochondrial (or plastid) genomes wherein at least about 10%, about 20%, about 30%, about 40%, about 50%, about 60%, about 70%, about 80%, or about 90% of the plurality of mitochondrial (or plastid) genomes comprise the edited mitochondrial (or plastid) genome. In some embodiments, the selected cell may comprise a plurality of mitochondrial (or plastid) genomes wherein at most about 20%, about 30%, about 40%, about 50%, about 60%, about 70%, about 80%, about 90%, or about 100% of the plurality of mitochondrial (or plastid) genomes comprise the edited mitochondrial (or plastid) genome. In some embodiments, an organelle can comprise a nucleus, a mitochondrion, a plastid, or a combination thereof.

In some embodiments, an expression level of a protein, an RNA, or a combination thereof can be higher when transformed into a plastid or mitochondrion as compared with that in a nucleus. In some embodiments, a protein and/or an RNA expression level can be at least about: 1%, 5%, 10%, 20%, 30%, 40%, 50%, 60%, 70%, 80%, or 90% higher with transformation of plastid or mitochondrial DNA as compared with a nuclear DNA transformation. In some embodiments, an expression stability of a protein, a transcript, or a combination thereof can be higher with a plastid or a mitochondrial transformation as compared with a nuclear transformation.

### Replacement Nucleic Acid (Donor Nucleic Acid) and Methods for Integrating a Donor Polynucleotide

In some embodiments, the disclosure provides methods to obtain an organelle (e.g., mitochondrion or plastid) comprising a donor polynucleotide (e.g., a replacement nucleic acid comprising an internal polynucleotide described herein). In some embodiments, a method can employ homologous recombination to provide integration of a polynucleotide at a target site. In some embodiments, a homologous recombination can be occurred without introducing a single or double-strand break via a site-directed nucleases (SDNs). In some embodiments, a homologous recombination can be enhanced by introducing a double-strand break (DSBs) at selected endonuclease target sites. In some embodiments, use of a site-directed nuclease can provide a high frequency of double-strand breaks at an organellar DNA target site. In some embodiments, a specific cleavage can enable efficient gene editing or replacement of a nucleotide sequence of interest. In some embodiments, a nucleotide sequence of interest to be edited or replaced can be located within or outside a target site recognized and/or cleaved by a site-directed nuclease. In some embodiments, the “donor polynucleotide” or “replacement nucleic acid” may comprise DNA and be referred to as a “donor DNA” or may comprise RNA and be referred to as a “donor RNA”. In some embodiments, a donor polynucleotide may be either single-stranded or double-stranded.

In some embodiments, the donor polynucleotide (e.g., a replacement nucleic acid) can comprise a polynucleotide encoding a variant of a naturally occurring polypeptide having an enzyme activity (e.g., an herbicide-resistant ALS, an herbicide-resistant EPSPS, or an herbicide-resistant GS). In some embodiments, the donor polynucleotide can comprise the polynucleotide encoding the variant of the naturally occurring polypeptide having the enzyme activity, and an additional polynucleotide encoding a gene of interest. In some embodiments, a gene of interest can be a cytoplasmic male sterility (CMS) coding region. In some embodiments, a donor polynucleotide can be inserted into a genome, e.g., at a cleavage site of a site-directed nuclease. In some embodiments, a donor polynucleotide can be inserted into a genome by homologous recombination. In some embodiments, the method further comprises removing the polynucleotide encoding the variant of the naturally occurring polypeptide having the enzyme activity after integration of the gene of interest.

In some embodiments, a donor polynucleotide (e.g., a replacement nucleic acid) of any suitable size can be integrated into a genome. In some embodiments, a donor polynucleotide integrated into a genome can be less than 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, about 500 kb, or less than about 500 kilobases (kb) in length. In some embodiments, a donor polynucleotide integrated into a genome can be at least about 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, about 500 kb, or less than about 500 kilobases (kb) in length. In some embodiments, a donor polynucleotide integrated into a genome can be up to about 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, or up to about 500 kb in length.

In some embodiments, an endogenous target polynucleotide to be replaced by a donor polynucleotide (e.g., a replacement nucleic acid) can be at least about 20 base pair (bp), at least about 50 bp, at least about 75 bp, at least about 100 bp, at least about 150 bp, at least about 250 bp, at least about 300 bp, at least about 400 bp, at least about 500 bp, at least about 600 bp, at least about 700 bp, at least about 800 bp, at least about 900 bp, at least about 1000 bp, at least about 1250 bp, at least about 1500 bp, at least about 1750 bp, at least about 2000 bp, at least about 2.5 kb, at least about 3 kb, at least about 4 kb, at least about 5 kb, at least about 6 kb, at least about 7 kb, at least about 8 kb, at least about 9 kb, at least about 10 kb, at least about 20 kb, at least about 50 kb, at least about 100 kb, at least about 200 kb, at least about 300 kb, at least about 400 kb, or at least about 500 kb in length.

In some embodiments, an endogenous target polynucleotide to be replaced by a donor polynucleotide (e.g., a replacement nucleic acid) can be less than about 50 base pair (bp), less than about 75 bp, less than about 100 bp, less than about 150 bp, less than about 250 bp, less than about 300 bp, less than about 400 bp, less than about 500 bp, less than about 600 bp, less than about 700 bp, less than about 800 bp, less than about 900 bp, less than about 1000 bp, less than about 1250 bp, less than about 1500 bp, less than about 1750 bp, less than about 2000 bp, less than about 2.5 kb, less than about 3 kb, less than about 4 kb, less than about 5 kb, less than about 6 kb, less than about 7 kb, less than about 8 kb, less than about 9 kb, less than about 10 kb, less than about 20 kb, less than about 50 kb, less than about 100 kb, less than about 200 kb, less than about 300 kb, less than about 400 kb, or less than about 500 kb in length.

In some embodiments, a donor polynucleotide (e.g., a replacement nucleic acid comprising an internal polynucleotide described herein) can comprise a polynucleotide of interest, a polynucleotide modification template, a heterologous expression cassette, or any combination thereof. In some embodiments, a donor polynucleotide (e.g., a replacement nucleic acid comprising an internal polynucleotide described herein) can encode a polypeptide selected from the group consisting of a cytoplasmic male sterility (CMS) polypeptide, a polypeptide with an herbicide-resistant enzyme activity, a polypeptide with an antibiotic-resistance enzyme activity, a polypeptide with phosphite dehydrogenase activity, and any combination thereof. In some embodiments, the term “polynucleotide modification template” can refer to a polynucleotide that can comprise at least one nucleotide modification when compared to a nucleotide sequence to be edited. In some embodiments, a nucleotide modification can be at least one nucleotide substitution, addition, deletion, or any combination thereof. In some embodiments, a minor genome modification created by use of a polynucleotide modification template can include creation of a mutant allele (e.g., antibiotic resistant rRNA gene) and removal of a target site for a polynucleotide guided polypeptide. In some embodiments, a donor polynucleotide (e.g., a replacement nucleic acid comprising an internal polynucleotide described herein) can comprise a first and a second region of homology. In some embodiments, a donor polynucleotide comprises a heterologous sequence that is flanked by a first and a second region of homology. In some embodiments, a first and second region of homology of a donor polynucleotide (e.g., a replacement nucleic acid comprising an internal polynucleotide described herein) can share homology to a first and a second genomic region, respectively, present in or flanking a target site (e.g., of an organellar genome).

In some embodiments, the CMS polypeptide comprises a rice orf79 polypeptide, a rice WA352 polypeptide, or both, and the plant cell is a rice cell. In some embodiments, the CMS polypeptide comprises a wheat orf256 polypeptide or a wheat orf279 polypeptide, or both, and the plant cell is a wheat cell. In some embodiments, a donor polynucleotide (e.g., a replacement nucleic acid comprising an internal polynucleotide described herein) can encode a polypeptide with herbicide-resistant enzyme activity, wherein the polypeptide is a plant-derived polypeptide. For example, in some embodiments, the herbicide-resistant enzyme activity is selected from the group consisting of an herbicide-resistant acetolactate synthase (ALS) activity, an herbicide-resistant glutamine synthetase (GS) activity, an herbicide-resistant 5-enol-pyruvyl-shikimate-3-phosphate synthase (EPSPS) activity, and any combination thereof. In some embodiments, a donor polynucleotide (e.g., a replacement nucleic acid comprising an internal polynucleotide described herein) can encode the polypeptide with the antibiotic-resistance enzyme activity. For example, in some embodiments, the polypeptide with the antibiotic-resistance enzyme can comprise an aminoglycoside adenylyltransferase (aadA) activity, an hygromycin phosphotransferase (hpt) activity, a neomycin phosphotransferase activity (nptII), and any combination thereof.

In some embodiments, a donor polynucleotide or a replacement nucleic acid described herein can comprise an internal polynucleotide, a 5′-homologous region (5′HR) polynucleotide and a 3′-homologous region (3′HR) polynucleotide. In some embodiments, a donor polynucleotide or a replacement nucleic acid described herein can comprise, in 5′ to 3′ direction, (i) the 5′-HR polynucleotide; (ii) the internal polynucleotide; and (iii) the 3′-HR polynucleotide. In some embodiments, the 5′-HR polynucleotide comprises a first sequence homologous to a genomic region adjacent to a first end of the endogenous target polynucleotide. In some embodiments, the 3′-HR polynucleotide comprises a second sequence homologous to a genomic region adjacent to a second end of the endogenous target polynucleotide.

In some embodiments, “homology” can mean DNA sequences that are similar. In some embodiments, homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology or identity. In some embodiments, a “region of homology to a genomic region” can be a region of DNA that has a similar sequence to a given “genomic region” in an organellar genome. In some embodiments, a region of homology can be of any length that can be sufficient to promote homologous recombination at a cleaved target site. In some embodiments, a region of homology can comprise at least 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that a region of homology can have sufficient homology to undergo homologous recombination with a corresponding genomic region. In some embodiments, a “sufficient homology” can indicate that two polynucleotide sequences can have sufficient structural similarity to act as substrates for a homologous recombination reaction. In some embodiments, a donor polynucleotide (e.g., a replacement nucleic acid comprising an internal polynucleotide described herein) may comprise an expression cassette (e.g., encoding a heterologous polynucleotide of interest). In some embodiments, a donor polynucleotide may comprise multiple expression cassettes. In some embodiments, an expression cassette may be a polycistronic expression cassette, e.g., where multiple protein-coding regions, functional RNAs, or a combination of both, are expressed under control of a single promoter.

In some embodiments, the method can further comprise introducing into a nucleus of the cell, a polynucleotide comprising a Rep coding region, and introducing into the mitochondrion or plastid of the cell a VOR-Donor-VOR polynucleotide. In some embodiments, a modified Rep protein comprising a Rep protein operably linked to a mitochondrial targeting peptide or a plastid targeting peptide can be introduced into the nucleus of the cell. In some embodiments, the modified Rep protein can comprise an amino acid sequence having at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 6. In some embodiments, the polynucleotide encoding a Rep protein or a modified Rep protein (e.g., nMTS-Rep) can be operably linked to an inducible promoter. In some embodiments, the polynucleotide encoding a Rep protein, or a modified Rep protein can be operably linked to a constitutively active promoter.

In some embodiments, the VOR-Donor DNA-VOR polynucleotide can comprise geminivirus VOR sequences such that the 5′ and 3′ Donor DNA regions with sequence homologous to target sites is flanked by a VOR sequences to yield a VOR-Donor DNA-VOR configuration. In some embodiments, the VOR sequence can comprise a nucleic acid sequence having at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 7.

In some embodiments, the Rep protein can induce replication of the donor DNA flanked by the VOR sequences, target sites for the geminivirus Rep protein. In some embodiments, amplification of the donor DNA mediated by Rep-VOR interaction, after its transformation into organelle, results in gene expression of the donor DNA being enhanced by at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400% or 500% compared to gene expression of the donor DNA lacking VOR elements, Rep protein, or both.

In some embodiments, modification of an organellar genome with a donor polynucleotide (e.g., a replacement nucleic acid described herein) can result in a replacement of part or all of an organelle DNA with a replacement nucleic acid. In some embodiments, a replacement nucleic acid may comprise in the 5′-to-3′ direction: (i) a 5′-homologous region (5′-HR) polynucleotide; (ii) an internal polynucleotide; and (iii) a 3′-homologous region (3′-HR) polynucleotide. The internal polynucleotide may comprise a sequence heterologous to the genome of the mitochondrion or the plastid. The 5′-HR polynucleotide may comprise a sequence with homology to a genomic region adjacent to one end of the endogenous polynucleotide to be replaced, and the 3′-HR polynucleotide may comprise a sequence with homology to a genomic region adjacent to the other end of the endogenous polynucleotide to be replaced. In some embodiments, an endogenous organellar DNA can be reduced or eliminated by use of a site-directed nuclease such as a TALEN, a meganuclease, a restriction enzyme, a polynucleotide guided nuclease (e.g., Cas9 polypeptide, MAD7 polypeptide), or any combination thereof. In some embodiments, an endogenous organellar DNA can be reduced or eliminated by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or 100% by use of a site-directed nuclease such as a TALEN, a meganuclease, a restriction enzyme, a polynucleotide guided nuclease (e.g., Cas9 polypeptide, MAD7 polypeptide), or any combination thereof. In some embodiments, a replacement nucleic acid can be introduced either before, at the same time, or after the introduction of a site-directed nuclease.

In some embodiments, the “replacement nucleic acid” may comprise DNA and be referred to as a “replacement DNA” or may comprise RNA and be referred to as a “replacement RNA”. In some embodiments, the term “replacement DNA” can refer to fragments of organellar DNA or complete organellar DNA that can convey a new genotype and corresponding trait(s) when transformed into an organelle. In some embodiments, the terms “replacement DNA” and “replacement organellar DNA” can be used interchangeably herein. In some embodiments, in the case of organellar DNA fragments, they can be integrated into a remaining endogenous organellar DNA by homologous recombination. In a case of complete organellar DNA replacement, a replacement DNA can be isolated from cultivars, lines, sub species and other species which possess DNA compositions distinct from an endogenous organellar DNA of recipient cells. In some embodiments, a replacement DNA can also be partially and/or completely synthesized in vitro. In some embodiments, a replacement DNA can comprise both native and non-native sequences. In some embodiments, when a replacement DNA is created in vitro, it can be a linear DNA with a repeat sequence at the ends. In some embodiments, a repeat sequence can be direct repeats or inverted repeats. In some embodiments, the ends can facilitate homologous recombination in vitro or in vivo to create circular DNA for replication of organellar DNA in cells. In some embodiments, a DNA created in vitro can also include heterologous DNA elements such as ones to allow selected amplification in bacterial cells. In some embodiments, a replacement DNA can comprise a DNA element functioning as a DNA replication origin in a recipient organelle. In some embodiments, a replacement DNA can comprise multiple DNA fragments that are capable of recombination within an organelle to result in a complete replacement DNA. In some embodiments, a replacement nucleic acid may be either single-stranded or double-stranded.

In some embodiments, a sequence functional as an origin of replication can be included with compositions (e.g., polynucleotides, constructs, cassettes) of the disclosure. Such sequences can include the origin of replication for an organelle. In some embodiments, an origin of replication sequence can be a plastid origin of replication (e.g., plastid rRNA intergenic region) sequence. In some embodiments, an origin of replication sequence can be a mitochondrial origin of replication sequence.

In some embodiments, as used herein, a “genomic region” can refer to a segment of DNA in a genome of, for example, an organelle (e.g., a mitochondrion or a plastid). In some embodiments, a genomic region can be present on either side of a target site. In some embodiments, a genomic region can comprise a portion of a target site. In some embodiments, a genomic region can comprise at least 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases. In some embodiments, a genomic region can comprise sufficient homology to undergo homologous recombination with a corresponding region of homology that is present on a donor DNA (e.g., a replacement nucleic acid comprising an internal polynucleotide as described herein).

In some embodiments, two or more polynucleotides encoding RNA and/or proteins can be included in a cassette as a polycistronic unit. In some embodiments, two or more polynucleotides encoding RNA and/or proteins can be expressed from separate cassettes.

In some embodiments, a guide polynucleotide/polypeptide system can be used for introducing one or more donor polynucleotides (e.g., a replacement nucleic acid comprising an internal polynucleotide described herein) or one or more traits of interest into one or more target sites by providing one or more guide polynucleotides, one or more SDNs (e.g., TALENs, Cas polypeptides, MAD polypeptides), and optionally one or more donor polynucleotides (e.g., replacement nucleic acids comprising internal polynucleotides) to a plant cell. In some embodiments, an organism can be produced from a cell that can comprise an alteration at said one or more target sites of an organellar DNA (e.g., mitochondrial DNA or plastid DNA), wherein an alteration can be selected from a group consisting of (i) a substitution of at least one nucleotide, (ii) a deletion of at least one nucleotide, (iii) an insertion of at least one nucleotide, and (iv) any combination of (i)-(iii).

In some embodiments, a structural similarity between a given genomic region and a corresponding region of homology of a donor polynucleotide (e.g., a replacement nucleic acid comprising an internal polynucleotide described herein) can be any degree of sequence identity that allows for homologous recombination to occur. In some embodiments, an amount of homology or sequence identity shared by a “region of homology” of a donor polynucleotide (e.g., donor DNA) and a “genomic region” of a plant genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

In some embodiments, a region of homology of a donor polynucleotide (e.g., a replacement nucleic acid comprising an internal polynucleotide described herein) can have homology to any sequence flanking a target site. While in some embodiments, regions of homology can share significant sequence homology to a genomic sequence immediately flanking a target site, the regions of homology can be designed to have sufficient homology to regions that may be further 5′ or 3′ to a target site. In still other embodiments, regions of homology can also have homology with a fragment of a target site along with downstream genomic regions. In one embodiment, a first region of homology can further comprise a first fragment of a target site and a second region of homology can comprise a second fragment of a target site, wherein a first and second fragments are dissimilar.

In some embodiments, as used herein, “homologous recombination” can refer to an exchange of DNA fragments between two DNA molecules at sites of homology. In some embodiments, a frequency of homologous recombination can be influenced by a number of factors. In some embodiments, a length of a region of homology can affect a frequency of homologous recombination events, for example, a longer a region of homology, can have a greater frequency of homologous recombination. In some embodiments, a length of a homology region needed to observe homologous recombination may vary among species.

In some embodiments, an intermolecular recombination can occur in mitochondria and in plastids, for example, plants with transformed mitochondrial DNA or transformed plastid DNA can arise through site-specific integration of foreign sequences by homologous recombination with a flanking sequence on a transformation vector.

In some embodiments, an intramolecular recombination between repeated sequences can generate, for example, inversions when repeats are palindromic or deletions when direct.

In some embodiments, endogenous mitochondrial or plastid sequences can be used to target insertions to achieve efficient foreign sequence integration by homologous recombination. In some embodiments, a positive correlation can be present between a rate of recombination and a length and/or degree of sequence homology.

In some embodiments, a minimum flanking sequence length for homologous recombination with an organellar genome can be influenced by an introduction of single-stranded or double-stranded breaks (or both) in an organellar genome, e.g., by polynucleotide guided polypeptide(s).

In some embodiments, an efficiency of a disclosed methods for genome engineering or modification can be at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100%.

In some embodiments, a method can comprise introducing into an organelle (e.g., a mitochondrion or a plastid) of a cell (e.g., a plant cell) a donor polynucleotide (e.g., a donor DNA), and a site-directed nuclease (e.g., a TALEN, a meganuclease, a restriction enzyme, a polynucleotide guided nuclease with one or more guide polynucleotides). In some embodiments, a first and/or a second region of homology of a donor polynucleotide (e.g., replacement nucleic acid comprising an internal polynucleotide) can undergo homologous recombination with their corresponding genomic regions of homology resulting in exchange of DNA between the donor and the genome. In some embodiments, the donor DNA (e.g., replacement nucleic acid comprising an internal polynucleotide) can be introduced into the cell either before, at the same time, or after the introduction of a site-directed nuclease. In some embodiments, methods disclosed herein can result in an integration of all or part of a donor polynucleotide (e.g., replacement nucleic acid comprising an internal polynucleotide) into an organellar genome such that an original genomic target site is either altered or removed, wherein an altered genomic target site is no longer recognized by the site-directed nuclease. In some embodiments, endogenous DNA not comprising all or part of a donor DNA can be eliminated by introduction of a site-directed nuclease.

In some embodiments, a cell can be a eukaryotic cell. In some embodiments, a cell can comprise, a human cell, an animal cell, a non-human animal cell, a bacterial cell, a fungal cell, an insect cell, a plant cell, a protist cell, a yeast cell, an algal cell, or any combination thereof. In some embodiments, a cell can be a wheat cell, a maize cell, a rice cell, a barley cell, a sorghum cell, a rye cell, a canola cell, a broccoli cell, a cauliflower cell, and a soybean cell. In some embodiments, a cell can be part of an organism or a tissue. In some embodiments, an organism can comprise a plant, a transgenic plant, or parts thereof comprising a cell, a tissue, a propagation material, a seed, a pollen, a progeny, or any combination thereof produced by the methods described herein. In some embodiments, a cell can be an isolated and purified human cell.

In some embodiments, a nucleotide to be edited can be located within or outside a target site recognized and cleaved by a site-directed nuclease. In some embodiments, at least one nucleotide modification may not be a modification at a target site recognized and cleaved by a site-directed nuclease. In some embodiments, there can be at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the organellar DNA target site. In some embodiments, a nucleotide to be edited can be located both within and outside a target site (or multiple target sites) recognized and cleaved by a site-directed nuclease.

In some embodiments, a donor polynucleotide (e.g., a replacement nucleic acid comprising an internal polynucleotide) can be introduced by any suitable means. In some embodiments, a plant having a target site can be provided. In some embodiments, a donor polynucleotide (e.g., a replacement nucleic acid comprising an internal polynucleotide) can be provided by any suitable transformation method including, for example, Agrobacterium-mediated transformation or biolistic particle bombardment. In some embodiments, a donor polynucleotide (e.g., a replacement nucleic acid comprising an internal polynucleotide) may be present transiently in a cell or it can be introduced via a viral replicon. In some embodiments, in a presence of a target site and a site-directed nuclease (e.g., a TALEN, a meganuclease, a restriction enzyme, a polynucleotide guided nuclease with one or more guide polynucleotides), all or part of a donor polynucleotide (e.g., a replacement nucleic acid comprising an internal polynucleotide) can be inserted into an organellar genome.

### Polynucleotides of Interest for Integration Into a Genome

In some embodiments, further provided are methods for identifying at least one plant cell comprising an organelle comprising a genome comprising a polynucleotide of interest integrated at a target site. In some embodiments, an organelle can comprise a mitochondrion, a plastid, or a combination thereof. In some embodiments, a donor polynucleotide can comprise a polynucleotide of interest. In some embodiments, a variety of methods can be used for identifying those plant cells with an insertion into a genome at or near to a target site without using a screenable marker phenotype. In some embodiments, a method can be viewed as directly analyzing a target sequence to detect any change in a target sequence, including but not limited to PCR methods, sequencing methods, nuclease digestion, Southern blots, and any combination thereof.

In some embodiments, a method can also comprise recovering a plant from a plant cell comprising a polynucleotide of interest integrated into its organellar genome. In some embodiments, a plant can be sterile or fertile.

In some embodiments, a polynucleotide or polypeptide of interest can comprise an herbicide-tolerance coding sequence, an insecticidal coding sequence, a nematocidal coding sequence, an antimicrobial coding sequence, an antifungal coding sequence, an antiviral coding sequence, an abiotic stress tolerance coding sequence, a biotic stress tolerance coding sequence, a sequence modifying a plant trait, or any combination thereof. In some embodiments, a plant trait can comprise yield, grain quality, nutrient content, starch quality and quantity, nitrogen fixation and/or utilization, and oil content and/or composition, or any combination thereof. In some embodiments, a polynucleotide of interest can include, a gene that improves crop yield, a polypeptide that improves a desirability of a crop, a gene encoding a protein conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms. In some embodiments, genes of interest can include, for example, those genes involved in information, such as zinc fingers, those involved in communication, such as kinases, and those involved in housekeeping, such as heat shock proteins. In some embodiments, a polynucleotide of interest can include a gene encoding an important trait for agronomics, insect resistance, disease resistance, herbicide resistance, fertility or sterility, grain characteristics, commercial products, or any combination thereof. In some embodiments, a gene of interest can include those involved in; oil, starch, carbohydrate, or nutrient metabolism; those affecting photosynthesis, photorespiration, and ATP metabolism; or any combination thereof.

In some embodiments, commercial traits can also be obtained by expression of proteins encoded on a polynucleotide. In some embodiments, a commercial use of transformed plants can be a production of polymers and bioplastics. In some embodiments, polynucleotides of interest can include genes encoding proteins such as β-ketothiolase, PHBase (polyhydroxybutyrate synthase), and acetoacetyl-CoA reductase which can facilitate expression of polyhydroxyalkanoates (PHAs). In some embodiments, a commercial use can be expression of a gene or genes that can increase starch for ethanol production.

In some embodiments, a polynucleotide or polypeptide that can influence amino acid biosynthesis can include, for example, anthranilate synthase (AS; EC 4.1.3.27) which can catalyze a first reaction branching from an aromatic amino acid pathway to a biosynthesis of tryptophan in plants, fungi, and bacteria. In some embodiments, in plants, a chemical process for a biosynthesis of tryptophan can be compartmentalized in a chloroplast. In some embodiments, additional donor sequences of interest can include Chorismate Pyruvate Lyase (CPL) which can refer to a gene encoding an enzyme which can catalyze a conversion of chorismate to pyruvate and pHBA. In some embodiments, a CPL gene can be from E. coli. In some embodiments, a CPL gene can bear GenBank accession number M96268.

In some embodiments, a polynucleotide sequence of interest can encode proteins involved in providing disease or pest resistance. In some embodiments, “disease resistance” or “pest resistance” can cause a plant to at least in part avoid a harmful symptom or outcome from a plant-pathogen interaction. In some embodiments, a pest resistance gene can encode resistance to a pest that has great yield drag. In some embodiments, a pest that has great yield drag can comprise rootworm, cutworm, European Corn Borer, or any combination thereof. In some embodiments, a disease resistance or insect resistance gene can comprise a lysozyme, a cecropin, or a combination thereof. In some embodiments, a disease resistance or insect resistance gene can provide antibacterial protection, antifungal protection, nematode protection, insect protection, or any combination thereof. In some embodiments, an antifungal resistance gene or protein can comprise a defensin, a glucanase, a chitinase or any combination thereof. In some embodiments, a nematode or insect protection gene or protein can comprise a Bacillus thuringiensis endotoxin, a protease inhibitor, a collagenase, a lectin, a glycosidase, or any combination thereof. In some embodiments, a gene encoding a disease resistance trait can include a detoxification gene. In some embodiments, a detoxification gene can comprise a fumonisin gene; an avirulence (avr) gene, a disease resistance (R) gene, or any combination thereof. In some embodiments, an insect resistance gene can encode resistance to pests that have great yield drag such as rootworm, cutworm, European Corn Borer, or any combination thereof. In some embodiments, an insect resistance gene can comprise a Bacillus thuringiensis (Bt) toxic protein gene.

In some embodiments, transgenes, recombinant DNA molecules, DNA sequences of interest, or donor polynucleotides can comprise one or more DNA sequences for gene silencing of a target gene. In some embodiments, a target gene can comprise a plant pest gene or a plant pathogen gene. In some embodiments, a method for gene silencing can comprise expression of a DNA sequence in a plant. In some embodiments, a method for gene silencing can comprise cosuppression, antisense suppression, double-stranded RNA (dsRNA) interference, hairpin RNA (hpRNA) interference, intron-containing hairpin RNA (ihpRNA) interference, transcriptional gene silencing, and microRNA (miRNA) interference.

In some embodiments, a fertile plant can be a plant that can produce viable male and female gametes and can be self-fertile. In some embodiments, a self-fertile plant can produce a progeny plant without a contribution from any other plant of a gamete and a genetic material contained therein. Also disclosed herein in some embodiments, are methods comprising a use of a plant that may not be self-fertile. In some embodiments, a plant may not produce male gametes, or female gametes, or both, that are viable or otherwise capable of fertilization. In some embodiments, as used herein, a “male-sterile plant” can be a plant that does not produce male gametes that are viable or otherwise capable of fertilization. In some embodiments, as used herein, a “female-sterile plant” can be a plant that does not produce female gametes that are viable or otherwise capable of fertilization. In some embodiments, male-sterile and female-sterile plants can be female-fertile and male-fertile, respectively. In some embodiments, a male-fertile (but female-sterile) plant can produce viable progeny when crossed with a female-fertile plant. In some embodiments, a female-fertile (but male-sterile) plant can produce viable progeny when crossed with a male-fertile plant. In some embodiments, in some crop species a use of hybrid plants has been shown to dramatically increase crop yield. In some embodiments, a hybrid crop system can require a male sterile line that can serve as a female parent to produce hybrid seed through fertilization with pollen donor plants. In some embodiments, a method to convey male sterility without manual or mechanical intervention can comprise a use of a cytoplasmic male sterility (CMS) gene. In some embodiments, a CMS gene can comprise a nucleic acid. In some embodiments, a CMS gene can comprise a heterologous nucleic acid. In some embodiments, a nucleic acid can comprise DNA, RNA, or a combination thereof. In some embodiments, a CMS gene can comprise a coding region, an open reading frame, or a combination thereof. In some embodiments, a CMS gene can be a maternally inherited trait conferred by a mitochondrial genome that results in a failure to produce functional pollen and/or male reproductive organs except in a presence of restorer-of-fertility (RF) genes. In some embodiments, a chimeric mitochondrial ORF can be found to lead to male sterility, producing unisex-female plants. In some embodiments, creation of a chimeric CMS gene can be a consequence of the highly recombinogenic, repetitive nature of plant mitochondrial genomes. In some embodiments, methods described herein could be used to introduce one or more naturally occurring or custom-designed CMS protein-coding sequences into mitochondria of various monocot species, dicot species, or a combination thereof. In some embodiments, a monocot species can comprise wheat, maize, rice, barley, sorghum, sugarcane, or rye. In some embodiments, a dicot species can comprise soybean, potato, tomato, canola, broccoli, or cauliflower. In some embodiments, a CMS protein-coding sequence of a CMS gene can be operably linked to an endogenous regulatory sequence, a heterologous regulatory sequence, or any combination thereof.

In some embodiments, a CMS gene may be from a cultivated rice (Oryza sativa), from a wild relative of rice (Oryza rufipogon), from a cultivated wheat (Triticum aestivum), or from a wild relative of wheat (Triticum timopheevii or Hordeum chilense). In some embodiments, a CMS gene can comprise all or part of an orf79 gene (e.g., an orf79 protein-coding sequence) from rice. In some embodiments, the CMS gene may encode an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 8. In some embodiments, a CMS gene can comprise all or part of an WA352 gene (e.g., a WA352 protein-coding sequence) from rice. In some embodiments, the WA352 gene may be a WA352a, a WA352b, or a WA352c gene. In some embodiments, the CMS gene may encode an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 128. In some embodiments, a CMS gene can comprise all or part of an orf256 gene (e.g., an orf256 protein-coding sequence) from wheat. In some embodiments, the CMS gene may encode an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 9. In some embodiments, a CMS gene can comprise all or part of an orf279 gene (e.g., an orf279 protein-coding sequence) from wheat. In some embodiments, the CMS gene may encode an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 10. In some embodiments, a CMS gene can comprise all or part of a T-urf13 gene (e.g., a T-urf13 protein-coding sequence) from maize.

### Selection of Transformed Cells Using Inhibitors of Plant Enzymes

In some embodiments, an embryogenic callus culture of a plant can be initiated and maintained for a minimum of 4-8 weeks (e.g., 4-6 weeks) on a Chu-N6-based induction & maintenance medium supplemented with the plant growth regulator 2,4-D. In some embodiments, the plant may be selected from the group consisting of: rice, wheat, maize, sorghum, barley, rye, canola, broccoli, cauliflower, and soybean. In some embodiments, the plant is rice. In some embodiments, four days prior to transformation, calli can be prepared for transformation by plating tissue in the target zone on the same N6-based medium supplemented with mannitol and sorbitol for osmotic protection.

In some embodiments, a plant callus (e.g., a rice callus) can be transformed with one or with multiple plant enzyme expression constructs (e.g., herbicide-resistant plant enzyme; regulatory plant enzyme). In some embodiments, a plant callus can be transformed with one or more plant enzymes (e.g., an herbicide-resistant ALS, an herbicide-resistant EPSPS, and/or an herbicide-resistant GS expression constructs). For example, the herbicide-resistant GS gene or EPSPS gene can be co-transformed with an herbicide-resistant ALS gene. In some embodiments, the herbicide-resistant plant enzyme expression cassette is a mitochondrial expression cassette or a plastid expression cassette. In some embodiments, the expression cassette is a nuclear expression cassette that encodes a plant enzyme (or a variant of plant enzyme) fused with a mitochondrial targeting sequence or a plastid targeting sequence.

In some embodiments, a plant callus (e.g., a rice callus) can be transformed with one or with multiple ALS expression constructs (e.g., an herbicide-resistant ALS-LS; a regulatory ALS-SS). In some embodiments, the herbicide-resistant ALS-LS expression cassette is a mitochondrial expression cassette or is a plastid expression cassette. In some embodiments, the ALS-SS expression cassette is a mitochondrial expression cassette or is a plastid expression cassette. In some embodiments, the ALS-SS (or the herbicide-resistant ALS-LS) expression cassette is a nuclear expression cassette that encodes an ALS-SS (or the herbicide-resistant ALS-LS) fused with a mitochondrial targeting sequence or fused with a plastid targeting sequence.

In some embodiments, the herbicide-resistant ALS can comprise an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 11. In some embodiments, the polynucleotide encoding the herbicide resistant ALS can comprise a nucleic acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 12. In some embodiments, the modified regulatory subunit of the acetolactate synthase or the modified biologically active fragment thereof can comprise an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 13.

In some embodiments, a plant callus (e.g., a rice callus) can be transformed with one or with multiple EPSPS expression constructs (e.g., ATP1 promoter driving coding regions for an herbicide-resistant version of EPSPS). In some embodiments, the herbicide-resistant EPSPS expression cassette is a mitochondrial expression cassette or is a plastid expression cassette. In some embodiments, the herbicide-resistant EPSPS expression cassette is a nuclear expression cassette that encodes an herbicide-resistant EPSPS fused with a mitochondrial targeting sequence or fused with a plastid targeting sequence.

In some embodiments, the herbicide-resistant EPSPS can comprise an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 14. In some embodiments, the polynucleotide encoding herbicide resistant EPSPS can comprise a nucleic acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 15.

In some embodiments, a plant callus (e.g., a rice callus) can be transformed with one or with multiple GS expression constructs (e.g., ATP1 promoter driving coding regions for an herbicide-resistant version of GS). In some embodiments, the herbicide-resistant GS expression cassette is a mitochondrial expression cassette or is a plastid expression cassette. In some embodiments, the herbicide-resistant GS expression cassette is a nuclear expression cassette that encodes an herbicide-resistant EPSPS fused with a mitochondrial targeting sequence or fused with a plastid targeting sequence.

In some embodiments, the herbicide-resistant GS can comprise an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 16. In some embodiments, the polynucleotide encoding herbicide resistant GS can comprise a nucleic acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 17.

In some embodiments, a sequence encoding a start codon of the variant of a naturally occurring polypeptide having an enzyme activity (e.g., an herbicide-resistant ALS, an herbicide-resistant EPSPS, or an herbicide-resistant GS) can be replaced with a sequence encoding a mitochondrial RNA editing site. For example, the mitochondrial RNA editing site can comprise a rice mitochondrial nad4L RNA editing site (e.g., SEQ ID NO: 18), a rice mitochondrial cox2 RNA editing site (e.g., SEQ ID NO: 19), a wheat mitochondrial cox2 RNA editing site (e.g., SEQ ID NO: 20), or any combination thereof. In some embodiments, the rice mitochondrial nad4L RNA editing site described herein can have a nucleic acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 18.

In some embodiments, the rice mitochondrial cox2 RNA editing site described herein can have a nucleic acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 19.

In some embodiments, the wheat mitochondrial cox2 RNA editing site described herein can have a nucleic acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 20.

In some embodiments, transformation is performed using a technique selected from the group consisting of: microinjection, meristem transformation, electroporation, Agrobacterium-mediated transformation, viral based gene transfer, transfection, vacuum infiltration, carbon nanotube based delivery, biolistic particle bombardment, or any combination thereof. In some embodiments, transformation may be performed using biolistic particle bombardment. In some embodiments, a variation of a transformation condition can comprise varying particle size and amount. In some embodiments, a variation of a transformation condition can comprise varying the amount of DNA on the particle. In some embodiments, a variation in transformation conditions can be the concentration of a selective agent in the first selection after bombardment, or in subsequent selections. In some embodiments, the following steps can be followed for bombardment, culture, selection, and regeneration:

In some embodiments, the bombardment procedure can be performed essentially as described in the scientific literature (Hamada, H., Linghu, Q., Nagira, Y. et al. 2017 An in planta biolistic method for stable wheat transformation. Sci Rep 7, 11443).

In some embodiments, plasmid DNA and/or purified DNA fragments (1-5 μg per DNA; approx. 1 μg/μl), can be combined with 50 μl 50% glycerol containing 3 mg of 0.6 μm gold particles (Bio-Rad, USA), 20 μL 0.1 M spermidine, and 50 μL 2.5 M CaCl₂ in a 1.5-mL tube and mixed by continuously vortexing for 3 minutes. After incubation on ice for 10 minutes, the DNA-coated gold particles can be centrifuged at 10,000 rpm for 15 seconds and the supernatant can be removed. The pellet can then be washed with ethanol (250 μL, 100%) and vortexed for 2 minutes. After a second centrifugation at 10,000 rpm for 30 seconds, the supernatant can be removed, and the final pellet can be resuspended in ethanol (60 μL, 100%) by finger tapping the tube and gently pumping the suspension in the pipette tip just before use. Aliquots (10 μL) can be spread onto macrocarrier membranes (Bio-Rad, USA). The rest of the DNA prep can then be additionally distributed across the macrocarriers. The macrocarriers and open plates of rice callus tissue which can be arranged in a central circular “target” on filter paper placed on a high osmotic medium can then be allowed to dry in a laminar flow hood for 10 minutes prior to the start of bombardment. Bombardment of one shot per plate of rice callus can be conducted using a PDS-1000/He™ device (Bio-Rad, USA) with a target distance of 8 cm from the stopping plate. The vacuum in the chamber can be −28.5 inches of Hg and the helium pressure can be 1100 psi.

After bombardment, a callus can be incubated in darkness for 16-20 hours at 26° C., then clumps approximately 1-3 mm in size can be sub-cultured to selective media. In some embodiments, selective media are supplemented with an inhibitor of a plant enzyme (e.g., ALS).

In some embodiments, the plant enzyme can be ALS, and the inhibitor of ALS is a sulfonylurea. In some embodiments, the inhibitor of ALS is chlorsulfuron. In some embodiments, the selective media are supplemented with chlorsulfuron at a concentration of 20 nM-100 nM, 100 nM-1 μM, 1 μM-20 μM, or 20 μM-100 μM. In some embodiments, the plant enzyme can be EPSPS, and inhibitor of EPSPS is a glyphosate. In some embodiments, the selective media can be supplemented with glyphosate at a concentration of at least about 0.1 mM, 0.5 mM, 1.0 mM, 1.5 mM, 2 mM, 2.5 mM, 3 mM, 3.5 mM, 4 mM, 4.5 mM, 5 mM, 10 mM, 20 mM, 30 mM, 40 mM, 50 mM, 60 mM, 70 mM, 80 mM, 90 mM, 100 mM, or more. In some embodiments, the plant enzyme can be GS, and inhibitor of GS can be a glyphosate, a bialaphos, a phosalacine. In some embodiments, the selective media are supplemented with glufosinate at a concentration of at 1-1000 mg/L, 10-500 mg/L, 20-400 mg/L, 30-300 mg/L, 40-200 mg/L, or 50-100 mg/L.

Calli on selective media can then be returned to dark incubation for 2-3 weeks. After 2-3 weeks of dark incubation, small clumps approximately 1-3 mm in size can again be subcultured to fresh selective medium containing a plant enzyme inhibitor (e.g., chlorsulfuron) and incubated for approximately 2 weeks in a lighted plant growth chamber with a 16 hr light-8 hr dark photoperiod, at intensity of 60 μmoles per square meter per second, at 26° C. In some embodiments, additional rounds of subculturing to fresh selection medium after 2-week periods of maintenance in the light can be performed.

At the end of the second selection period or later, between 5-8 weeks after bombardment, vigorously growing calli (individual initial events) can be picked from the surrounding dying tissue and transferred to individual plates of fresh selective medium supplemented with the plant enzyme inhibitor (e.g., chlorsulfuron), maintaining their individual identity.

At the end of this last 2-week or more selection period of individual plates, calli which are sustaining growth (representing putative mitochondrial or plastid transformation events) can be transferred to an N6-based medium for embryo maturation, still containing the plant enzyme inhibitor (e.g., chlorsulfuron) as a selective agent, but omitting the growth regulator 2,4-D, and supplementing with 2.5 g/L Phytagel.

After a minimum of 10-14 days, mature somatic embryos showing signs of normal maturation can be transferred to an N6-based germination medium, still containing the plant enzyme inhibitor (e.g., chlorsulfuron) as a selective agent. In some embodiments, this medium can be supplemented with growth regulators 0.2 mg/L naphthaleneacetic acid and 2 mg/L 6-benzylamino purine, and 2.5 g/L Phytagel.

In some embodiments, these events can be grown in a continuous light growth environment at 26-28° C. for root and shoot formation. In some embodiments, these events can be grown in a 16 h/8 h light/dark growth chamber at 26-28° C. for root and shoot formation.

In some embodiments, plants showing both root and shoot development after the previous step may be transferred to pots containing an artificial potting medium and gently acclimatized to greenhouse conditions. The plants may be grown to maturity and seed production in a greenhouse.

In some embodiments, immature scutella of a plant can be used. Approximately twenty-four hours prior to transformation, immature scutella approximately 2 mm in length of wheat cultivars Fielder and/or Bobwhite can be prepared for transformation by excising them from immature seeds, removing the small embryo axis, and plating them in a circular target zone on a high-osmotic medium. In some embodiments, the medium can comprise an agar-solidified MS basal medium supplemented with amino acids, sucrose and 2,4-D, with or without the addition of cefotaxime antibiotic at the rate of 250 mg/L for contamination control.

In some embodiments, the precultured wheat scutella can be transformed with one or with multiple plant enzyme expression constructs (e.g., an herbicide-resistant plant enzyme; a regulatory plant enzyme) using the biolistics method (particle bombardment). In some embodiments, the scutella can be co-transformed with a mitochondrial oligomycin resistance gene (oliR) linked to the plant enzyme described herein.

In some embodiments, immediately after bombardment, or up to 2 days after bombardment, the scutella can be spread out across the bombarded plates or spaced out onto additional new plates of the same high osmotic medium and incubated in the dark for up to 7 days at 26° C. In some embodiments, cultured scutella can be transferred to a selective callus induction medium (e.g., the MS-based high osmotic medium supplemented, an inhibitor of the plant enzyme and/or cefotaxime). In some embodiments, the culture can be in dark incubation for at least up to 1, 2, 3, 4, 5 weeks.

In some embodiments, after incubation on selective callus induction medium containing the inhibitor of the plant enzyme, the scutella can be continuously maintained on the MS-based selective callus induction medium with the inhibitor of plant enzyme (e.g., chlorsulfuron). In some embodiments, after incubation on selective callus induction medium containing the inhibitor of the plant enzyme, the scutella can be transferred to a first stage agarose-solidified regeneration medium (RZ) supplemented with maltose, 2,4-D, zeatin and silver nitrate in presence of the inhibitor of the plant enzyme. In some embodiments, cefotaxime use can be discontinued after three to six weeks of culture on the callus induction medium.

In some embodiments, the scutella on callus induction medium can be cultured in the light (16/8 photoperiod) and transferred to fresh medium with the inhibitor of the plant enzyme (e.g., chlorsulfuron) approximately every three weeks for 32 weeks. In some embodiments, calli induced from individual bombarded scutella can be subdivided into smaller pieces and maintain their original identity.

In some embodiments, scutella on shoot induction medium can be sub-cultured to fresh first stage regeneration medium every three weeks and cultured in the light until shoot formation is visible. In some embodiments, selected green sectors of callus and small shoots can be transferred to a second stage regeneration medium (R0) which is the same as the first stage regeneration medium, but without growth regulators. Developing plants can be transferred to domed clear culture vessels and grown on to transplantable size. In some embodiments, the developing plants can be transplanted to soil and acclimatized in the greenhouse.

### Dual Selection Process

In some embodiments, the methods described herein can have multiple selection processes. In some embodiments, the gene expression cassette comprising a plant enzyme or a variant thereof (e.g., an herbicide-resistant ALS) can be co-transformed with a second selectable marker expression cassette, for example, a 35S: HPT nuclear expression cassette conferring hygromycin (e.g., hygromycin B) resistance. In some embodiments, a selective medium can comprise one or more inhibitors of the plant enzyme and selective agents (e.g., hygromycin or hygromycin B). In some embodiments, a selective medium can comprise one or more inhibitors of the plant enzyme (e.g., chlorsulfuron) and at least about 1-100 mg/L, 10-75 mg/L, or 25-50 mg/L of hygromycin (e.g., hygromycin B).

In some embodiments, the gene expression cassette comprising a plant enzyme or a variant thereof (e.g., a herbicide-resistant ALS) can be also linked and co-transformed with an oliR expression cassette, conferring resistance to the antibiotic oligomycin. In some embodiments, oligomycin can be incorporated into the selective medium at about 0.5-100 mg/L, 1-50 mg/L, 1-50 mg/L, or 1-5 mg/L. In some embodiments, the carbon source (e.g., sucrose) can be reduced to about 0.1% (1 mg/L), 0.2% (2 mg/L), 0.3% (3 mg/L), 0.4% (4 mg/L), 0.5% (5 mg/L) 0.6% (6 mg/L), 0.7% (7 mg/L), 0.8% (8 mg/L), 0.9% (9 mg/L) or replaced with at least about 10 mL, 20 mL, 30 mL, 40 mL, 50 mL, 60 ml, 70 mL, 80 mL, 90 mL, or 100 mL of a sterile 50% glycerol solution per L of selective medium to enhance the effectiveness of oligomycin.

In some embodiments, the compound disulfiram can be also incorporated into the selective medium at about 20 μM, 40 μM, 60 μM, 800 M, 100 μM, 150 μM, 200 μM, 300 μM, 400 μM, or 500 μM, to inhibit the ability of cells to utilize any alcohol produced by anaerobic respiration of treated cells. In some embodiments, the gene expression cassette comprising a plant enzyme or a variant thereof (e.g., an herbicide-resistant ALS gene) can be linked to geminivirus VOR sequences.

In some embodiments, the gene expression cassette comprising a plant enzyme or a variant thereof (e.g., an herbicide-resistant ALS) can be co-transformed with a second selectable marker expression cassette comprising a polynucleotide encoding a phosphite dehydrogenase enzyme or a biologically active fragment thereof. In some embodiments, the selective medium can comprise one or more inhibitors of the plant enzyme and selective agents (e.g., phosphite). In some embodiments, the selective medium can comprise one or more inhibitors of the plant enzyme (e.g., chlorsulfuron) and at least about 0.1-0.25 mM, 0.25-0.5 mM, 0.5-0.75 mM, 0.75-1.0 mM, 1.0-2.5 mM, 2.5-5.0 mM, 5.0-7.5 mM, 7.5-10 mM, 10-15 mM, 15-20 mM, 20-25 mM, 25-30 mM, 30-35 mM, 35-40 mM, 40-45 mM, and 45-50 mM.

In some embodiments, the gene expression cassette comprising the plant enzyme or a variant thereof (e.g., an herbicide-resistant ALS gene) can be co-transformed with at least one expression cassette conferring resistance to an additional selection marker (e.g., conferring hygromycin or hygromycin B resistance or oligomycin resistance), at least two expression cassettes (e.g., conferring hygromycin or hygromycin B resistance and oligomycin resistance), or at least three expression cassettes (e.g., conferring hygromycin or hygromycin B resistance, oligomycin resistance and an additional selection agent). In some embodiments, one or more selective agents (e.g., hygromycin, hygromycin B, oligomycin) can be added in place of, or in addition to, the inhibitor of the plant enzyme. In some embodiments, variations can be made in the timing of the inception of one or more selection agents in conjunction with an inhibitor of the plant enzyme (e.g., chlorsulfuron). For example, after an initial period of one or more cycles of subculture and selection, the use of the inhibitor of the plant enzyme can be discontinued and one or more selection agents (e.g., hygromycin, hygromycin B, oligomycin) can be utilized. Alternatively, after an initial period of one or more cycles of subculture and selection, the use of one or more selection agents (e.g., hygromycin, hygromycin B, oligomycin) can be discontinued and the inhibitor of the plant enzyme can be utilized.

### Inducible Expression System

In some embodiments, the target tissue for transformation (e.g., biolistics transformation) of the plant (e.g., rice) can be from a different source. The different source of callus tissue can be derived from a previous Agrobacterium tumefaciens transformation. In some embodiments, a dexamethasone-inducible system can be used to produce a geminivirus Rep protein. In some embodiments, to supply enough tissue for bombardment, the event can be maintained on the first selective medium, which can be supplemented with 40 mg/L hygromycin, amino acids, proline, maltose and 2,4-D growth regulator prior to the transformation.

In some embodiments, the tissue to be bombarded can be derived from the inducible line, precultured for 4 days prior to bombardment on the first selective medium. In some embodiments, the preculture medium can be also supplemented with 1,000 μl of 10 μM dexamethasone (DEX). In some embodiments, DMSO can be used as a control.

In some embodiments, calli can be prepared for bombardment by plating tissue in the target zone on the N6-based callus induction medium supplemented with mannitol and sorbitol for osmotic protection, but without DEX. After bombardment, the calli can be incubated in the dark for 16-20 hours at 26° C., then clumps of callus tissue approximately 1-3 mm in size can be sub-cultured to the N6-based callus maintenance medium supplemented with growth regulator 2,4-D and the appropriate selective agents including appropriate an inhibitor of the plant enzyme (e.g., 20-100 nM chlorsulfuron) and one or more additional selection marker (e.g., 25-50 mg/L hygromycin and 1-5 mg/L oligomycin) with reduced or alternate carbon source. In some embodiments, chemical induction with DEX in the medium can begin with the first round of selection. Calli on selective media with DEX can be then returned to dark incubation for the first round of selection. In some embodiments, DEX was introduced at a later point in the selection process. After 2-3 weeks of dark incubation, small (1-3 mm) clumps can be again sub-cultured to a fresh selective medium and then incubated as described. Additional rounds of sub-culturing to fresh selection medium with DEX and/or DMSO after 2 to 3-week periods of maintenance in the light can be most performed. In some embodiments, the induction with DEX can be continued throughout the entire selection process. In some embodiments, the use of DEX and/or DMSO can be discontinued for one or more culture periods. In some experiments, DEX can be re-introduced at a later point in the selection process.

### Screenable and Selectable Markers

In some embodiments, a polynucleotide (e.g., a donor polynucleotide; a replacement nucleic acid comprising an internal polynucleotide) can also encode a phenotypic marker. In some embodiments, a phenotypic marker can be a screenable or a selectable marker that can include a visual screenable marker, a selectable marker, or a combination thereof. In some embodiments, a selectable marker can comprise a positive or negative selectable marker. In some embodiments, any phenotypic marker can be used. In some embodiments, a selectable or screenable marker can comprise a DNA segment that can allow one to identify or select for or against a molecule or a cell that contains it, e.g., under particular conditions. In some embodiments, a marker can encode an activity, such as, but not limited to, production of RNA, peptide, or protein, or can provide a binding site for RNA, peptides, proteins, inorganic and organic compounds or compositions and the like.

In some embodiments, an example of a selectable or screenable marker can include, but are not limited to, DNA segments that comprise restriction enzyme sites; DNA segments that encode products which provide resistance against otherwise toxic compounds including antibiotics, such as, spectinomycin, ampicillin, kanamycin, tetracycline, hygromycin; DNA segments that encode products which are otherwise lacking in a recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan fluorescent protein (CFP), yellow fluorescent protein (YFP), red fluorescent protein (RFP), and cell surface proteins); the generation of new primer sites for PCR (e.g., the juxtaposition of two DNA sequence not previously juxtaposed); the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification, or any combination thereof.

In some embodiments, additional selectable markers can include polynucleotides that encode proteins that can confer resistance/tolerance to herbicidal compounds, such as glyphosate, sulfonylureas, glufosinate ammonium, bromoxynil, imidazolinones, and 2,4-dichlorophenoxyacetate (2,4-D). In some embodiments, herbicide resistance proteins can include herbicide tolerant versions of the following: an acetyl coenzyme A carboxylase (ACCase); a 4-hydroxyphenylpyruvate dioxygenase (HPPD); an acetolactate synthase (ALS); a glutamine synthetase (GS); a 5-enolpyruvylshikimate-3-phosphate synthase (EPSPS); a protoporphyrinogen oxidase (PPO or PROTOX); an auxin enzyme or receptor; or any combination thereof. In some embodiments, herbicide resistance proteins can include herbicide inactivating enzymes such as the following: a glyphosate oxidoreductase (GOX); a glyphosate N-acetyltransferase (GAT); a phosphinothricin acetyl transferase (PAT); a dicamba monooxygenase; a P450 polypeptide; or any combination thereof. In some embodiments, non-limiting examples of genes useful for conferring herbicide resistance in plants can include genes that encode the above proteins. In some embodiments, a neomycin phosphotransferase II (nptII) gene can encode a protein to provide resistance to antibiotics kanamycin and geneticin and a hygromycin phosphotransferase (HPT) gene can encode a protein to provide resistance to hygromycin.

In some embodiments, a DNA transformation of organellar genomes can be performed, for example, in plastids and mitochondria. In some embodiments, a selectable marker gene can include, for example, genes for photosynthesis (atpB, tscA, psaA/B, petB, petA, ycf3, rpoA, rbcL), antibiotic resistance (rrnS, rrnL, aadA, nptII, aphA-6), herbicide resistance (psbA, bar, AHAS (ALS), EPSPS, HPPD, sul) and metabolism (BADH, codA, ARG8, ASA2) genes. In some embodiments, a sul gene from bacteria can encode a protein with herbicidal sulfonamide-insensitive 7,8-dihydropteroate synthase activity and can be used as a selectable marker when a protein product is targeted to, or expressed within, plant mitochondria. In some embodiments, a sul gene can be a sul1 gene, a sul2 gene, a sul3 gene, a sul4 gene, or any functional fragment thereof. In some embodiments, a folk gene may be used with a sul gene. In some embodiments, a folk gene can encode a protein with 6-hydroxymethyl-7,8-dihydropterin pyrophosphokinase activity. In some embodiments, a protein product with folk activity is targeted to, or expressed within, plant mitochondria. In some embodiments, a gene encoding a bifunctional protein having both sul and folk activities may be used.

In some embodiments, a sequence encoding a marker can be incorporated into a genome of an organelle. In some embodiments, an incorporated sequence encoding a marker can be subsequently removed from a transformed organellar genome. In some embodiments, a removal of a sequence encoding a marker may be facilitated by a presence of direct repeats before and after a region encoding a marker. In some embodiments, removal of a sequence encoding a marker can occur via an endogenous homologous recombination system of an organelle or by use of a site-specific recombinase system such as cre-lox or a site-directed recombination method. In some embodiments, a site-directed recombination method can comprise FLP-FRT recombination.

In some embodiments, Caspase Activatable-GFP (CA-GFP) is a modified version of GFP in which fluorescence is completely quenched by appendage of a hydrophobic quenching peptide that tetramerizes GFP and prevents maturation of a chromophore. In some embodiments, a sequence of a CA-GFP protein can correspond to a GFP with a fusion of DEVDFQGPCNDSSDPLVVAASIIGILHLILWILDRL (SEQ ID NO: 21) at the carboxy terminus. In some embodiments, a sequence of a CA-GFP protein can correspond to a GFP with a fusion having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more sequence identity to SEQ ID NO: 21. In some embodiments, a caspase recognition sequence comprising the amino acids DEVD (SEQ ID NO: 22) can be present in CA-GFP between the fluorescence and the quenching domains. In some embodiments, GFP fluorescence can be fully restored in vivo by catalytic removal of a quenching peptide by cleavage with caspase. In some embodiments, a nucleic acid sequence encoding CA-GFP can be modified by replacement of a caspase recognition sequence with a mitochondrial RNA editing sequence. In some embodiments, an RNA editing sequence can be selected such that a C-to-U conversion results in creation of a stop codon in an mRNA. In some embodiments, expression of a nucleic acid sequence encoding a modified CA-GFP would result in quenching in a cytoplasm or in plastids but would produce fluorescence in mitochondria, thus providing a screenable marker. In some embodiments, a candidate RNA editing sequence for this purpose is present in a wheat mitochondrial cox2 gene at positions 449, 587 and 620 of a gene. In some embodiments, a candidate RNA editing sequence for this purpose that is present in a wheat mitochondrial cox2 gene at positions 449, 587 and 620 of a gene can comprise SEQ ID NO: 23, SEQ ID NO: 24, and SEQ ID NO: 25, respectively. In some embodiments, a candidate RNA editing sequence present in a wheat mitochondrial cox2 gene at positions 449, 587 and 620 of a gene can comprise a nucleic acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to SEQ ID NO: 23, SEQ ID NO: 24, and SEQ ID NO: 25, respectively.

Disclosed herein in some embodiments, are methods that can provide transformation efficiency into an organelle (e.g., mitochondria, plastids) of, for example, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99%, or at least about 100% transformation efficiency.

Use of Multiple Selectable and/or Screenable Markers

The systems and methods described herein may utilize at least one, at least two, at least three, at least four, or at least five selectable or screenable markers. Commonly used selectable marker genes in plant may include, for example, genes that confer resistance to antibiotics, such as neomycin, kanamycin, and paromomycin (neomycin phosphotransferase II gene, nptII), hygromycin B (hygromycin phosphotransferase gene, hpt, aphIV), streptomycin or spectinomycin (aminoglycoside adenylyltransferase gene, aadA) and gentamicin (aac3 and aacC4), or those that provide resistance to herbicides such as glufosinate (phosphinothricin acetyltransferase gene, bar or pat), dicamba (DMO) and glyphosate (aroA or EPSPS). In some cases, a screenable marker may provide an ability to visually screen transformants such as luciferase or green fluorescent protein (GFP), or genes expressing known uidA genes (GUS) or beta glucuronidase of various chromogenic substrates. In some embodiments, one or more selectable or screenable markers may be used at different growth stages of a cell, a tissue, a propagation material, a seed, a pollen, a progeny, or any combination thereof. For example, a cell may be co-transformed with a first selectable marker (e.g., a gene that confers resistance to the antibiotic hygromycin) and a second selectable maker (e.g., an herbicide-resistant ALS-LS), and may grow in a presence of a first selective agent (hygromycin) and then subsequently in a presence of a second selective agent (e.g., an inhibitor of ALS) at different growth stages. The transformation may also be performed in the absence of selection during one or more stages or steps of development or regeneration of the transformed cell, tissue, propagation material, seed, pollen, progeny, or any combination thereof. In some embodiments, one or more selectable or screenable markers may be incorporated in different organelles (e.g., nuclear and mitochondrial genomes). In some embodiments, one or more selectable or screenable markers may be removed upon successful transformation.

Herbicide-resistant ALS Large Subunit as a Selectable Marker

The acetolactate synthase large subunit (ALS-LS; EC: 2.2.1.6) catalyzes the first common step of the biosynthetic pathway for the synthesis of the branched-chain amino acids leucine, isoleucine, and valine. Acetolactate synthase activity comprises conversion of two molecules of pyruvate to one molecule of acetolactate and one molecule of carbon dioxide. Acetolactate synthase is also known as acetohydroxyacid synthase (AHAS). In some cases, acetolactate synthase also has a regulatory small subunit (ALS-SS). In some cases, the regulatory small subunit stimulates activity of the acetolactate synthase catalytic large subunit seven- to ten-fold and confers sensitivity to inhibition by valine and activation by ATP. In yeast, ALS is in the mitochondria. In yeast, the genes for the large and small subunits are encoded in the nucleus and the primary translation products have mitochondrial targeting sequences. In plants and algae, ALS is in the plastid. In plants and algae, the genes for the large and small subunits of ALS are encoded in the nucleus and the primary translation products have plastid targeting sequences.

In some cases, inhibitors of ALS are used as herbicides by inhibiting the production of branch-chain amino acids. These inhibitors are not a chemistry class but rather a mechanism class having diverse chemistries. The ALS inhibitor family includes sulfonylureas (SUs), imidazolinones (IMIs), triazolopyrimidines (TPs), pyrimidinyl benzoates (PYBs), sulfonanilide, and sulfonylamino carbonyl triazolinones (SCTs). ALS herbicides do not bind to the catalytic site but instead at a site specific to herbicidal action. Consequently, resistance mutations have shown widely varying effects on normal ALS catalysis activity, i.e., positive, negative, and neutral. For example, resistance in Hordeum murinum due to a proline-to-serine substitution at amino acid 197 was found to increase ALS activity by 2-to-3 fold. Herbicide-resistance to ALS has been identified in many weed species.

In some embodiments, an herbicide-resistant ALS-LS can have the following: altered Km, altered Vmax, altered cofactor affinity, altered cofactor specificity, altered thermostability, altered feedback regulation, or any combination thereof.

In some embodiments, an herbicide-resistant ALS-LS can be identified from an herbicide-resistant weed population. In some embodiments, an herbicide-resistant weed population can be from the following species: Xanthium strumarium, Kochia scoparia, Amaranthus hybridus, Apera spica-venti, Amaranthus powellii, and Oryza sativa var. sylvantica. In some embodiments, an herbicide-resistant ALS-LS can have at least one mutation at any of the following amino acids: Ala-122, Pro-197, Ala-205, Asp-376, Arg-377, Trp-574, Ser-653, Gly-654, and any combination thereof. In some cases, the amino acid number is standardized to the Arabidopsis thaliana sequence.

In some embodiments, an herbicide-resistant ALS-LS can be intentionally selected, for example, by laboratory selections. In some embodiments, an herbicide-resistant population can be from the following species: Oryza sativa, Zea mays, Arabidopsis thaliana, Camelina sativa, Sorghum bicolor. In some embodiments, an herbicide-resistant ALS-LS can be from Oryza sativa and can have at least one of the following amino acid mutations: Trp548Leu, Ser627Ile, Trp548Met, Ser627Asn, and any combination thereof. In some embodiments, an herbicide-resistant ALS-LS can be from Zea mays and can have at least one of the following amino acid mutations: Pro165Ser, Pro165Ala, Pro165Leu, Pro165Trp. In some embodiments, an herbicide-resistant ALS-LS can be from Arabidopsis thaliana and can have at least one of the following amino acid mutations: Ser653Asn, Ala122Thr, Ala122Val, Ala205Val, Trp574Ser, Trp574Leu, Ser653Asn, Pro197Ala, and any combination thereof. In some embodiments, an herbicide-resistant ALS-LS can be from Camelina sativa and can have at least one of the following amino acid mutations: Alal22Thr, Pro197Ser, Trp574Leu, and any combination thereof. In some embodiments, an herbicide-resistant ALS-LS can be from Sorghum bicolor and can have at least one of the following amino acid mutations: Val531Ile, Trp545Leu, and any combination thereof.

In some embodiments, an herbicide-resistant ALS-LS can be of plant origin. In some embodiments, an herbicide-resistant ALS-LS can be lacking a chloroplast transit sequence. In some embodiments, a polynucleotide encoding an herbicide-resistant ALS-LS lacking a chloroplast transit sequence can be introduced into the mitochondria. In some embodiments, an enzyme can comprise both an herbicide-resistant ALS-LS and a regulatory ALS-SS. In some embodiments, an ALS-SS can be of plant origin. In some embodiments, an ALS-SS can be lacking a chloroplast transit sequence. In some embodiments, an ALS-SS is fused to a mitochondrial targeting sequence. In some embodiments, a polynucleotide encoding an ALS-SS fused to a mitochondrial targeting sequence can be introduced into the nuclear genome. In some embodiments, a polynucleotide encoding an ALS-SS lacking a chloroplast transit sequence can be introduced into the mitochondria. In some embodiments, an herbicide-resistant ALS-LS can be from Oryza sativa. In some embodiments, an ALS-SS can be from Oryza sativa. In some embodiments, the presence of an herbicide-resistant ALS-LS in mitochondria can enable synthesis in the cell of branched-chain amino acids (valine, leucine, and isoleucine) in the presence of an inhibitor of acetolactate synthase which can allow for its use as a selectable marker.

### Benefits of Organisms Having Mitochondria or Plastids Transformed to Express a Polypeptide Having Herbicide-Resistant Enzyme Activity

In some embodiments, introduction into a mitochondrion or a plastid of a polynucleotide encoding a polypeptide having herbicide-resistant enzyme activity can allow for selection of a plant cell or an algal cell having stably transformed mitochondria or plastids. In some embodiments, transformation of mitochondria or plastids with a polynucleotide encoding a polypeptide having herbicide-resistant enzyme activity can allow for co-transformation with an additional polynucleotide of interest. In some embodiments, at least 50%, 60%, 70%, 80%, 90%, 95% or 100% of the mitochondrial genomes or the plastid genomes in a cell can be transformed. In some embodiments, a cell can be homoplasmic for the transformed mitochondria or the transformed plastid.

In some embodiments, the method described herein may promote growth or cultivation of a plant of interest comprising the edited mitochondrial genome or the edited plastid genome, while suppressing the growth of an undesired plant (e.g., weed) that does not comprise the edited mitochondrial genome or the edited plastid genome. For example, a plurality of plants may be grown in a presence of an inhibitor of plant enzyme described herein (e.g., ALS, EPSPS, GS), wherein at least one desired plant of the plurality of plants comprises a mitochondrion or a plastid having a heterologous polynucleotide that encodes a polypeptide having herbicide-resistant plant enzyme activity or a biologically active fragment thereof and at least one undesired plant (e.g., weed) of the plurality of plants lacking a mitochondrion or a plastid having a heterologous polynucleotide that encodes a polypeptide having herbicide-resistant acetolactate synthase activity or a biologically active fragment thereof. In some embodiments, the presence of the inhibitor of the plant enzyme is sufficient to selectively promote growth of the at least one desired plant of the plurality of plants, resulting in an increased growth of the at least one desired plant of the plurality of plants relative to undesired plants (e.g., weed) lacking a polypeptide having herbicide-resistant acetolactate synthase activity or a biologically active fragment thereof. In some embodiments, the inhibitor of the plant enzyme may be applied to the plant, the plurality of plants, soil adjacent to the plants or any combination thereof. In some embodiments, the inhibitor of the plant enzyme is applied as a foliar amendment, a soil amendment, or any combination thereof. In some embodiments, the inhibitor of the plant enzyme may be dissolved in water and applied to the plant, the plurality of plants, soil adjacent to the plants or any combination thereof.

In some embodiments, a plant having mitochondria or plastids transformed with a polynucleotide encoding a polypeptide having herbicide-resistant plant enzyme activity can transmit the transformed mitochondria or the transformed plastids to progeny plants by maternal inheritance. In some embodiments, a plant having mitochondria or plastids transformed with a polynucleotide encoding a polypeptide having herbicide-resistant plant enzyme activity can have less horizontal gene transfer (e.g., to a weed species) than a plant having a nuclear genome transformed with a polynucleotide encoding a polypeptide having herbicide-resistant acetolactate synthase activity.

### Herbicide Resistance in Plants

In some embodiments, an “herbicide resistance protein” or a protein resulting from expression of an “herbicide resistance-encoding nucleic acid molecule” can include proteins that can confer upon a cell the ability to tolerate a higher concentration of an herbicide, for example, compared with cells that do not express the protein. In some embodiments, an herbicide resistance protein can have enzymatic activity. In some embodiments, an herbicide resistance protein can have enzymatic activity in the presence of an herbicide that targets said enzymatic activity. In some embodiments, an herbicide resistance protein can have enzymatic activity that results in degradation and/or inactivation of an herbicide. In some embodiments, an herbicide resistance protein can be monomeric or multimeric. In some embodiments, an herbicide resistance protein can comprise a single polypeptide. In some embodiments, an herbicide resistance protein can comprise two or more distinct polypeptides. In some embodiments, the terms “herbicide resistance protein”, “herbicide-resistant protein”, “herbicide tolerance protein” and “herbicide tolerant protein” may be used interchangeably herein when used to describe a molecule (e.g., a protein, an enzyme, a subunit, or a nucleic acid) that can confer upon a cell (or plant) the ability to tolerate a higher concentration of an herbicide compared with a cell (or plant) that does not express the molecule.

In some embodiments, an herbicide resistance protein or a protein resulting from expression of an herbicide resistance-encoding nucleic acid molecule can include proteins that can confer upon a cell an ability to tolerate a concentration of a herbicide for a longer period of time than cells that do not express a protein. In some embodiments, herbicide resistance traits may be introduced into plants by, for example, genes coding for resistance to herbicides. In some embodiments, genes coding for resistance to herbicides include, for example, the following: genes that act to convey tolerance to inhibitors of acetolactate synthase (ALS), which is also known as acetohydroxyacid synthase (AHAS), such as the sulfonylurea-type herbicides; genes (e.g., the bar gene, the pat gene) that act to convey tolerance to inhibitors of glutamine synthetase, such as phosphinothricin or basta; genes that act to convey tolerance to inhibitors of the EPSP synthase gene, such as glyphosate; genes that act to convey tolerance to inhibitors of HPPD; genes that act to convey tolerance to inhibitors of an acetyl coenzyme A carboxylase (ACCase); and genes that act to convey tolerance to inhibitors of protoporphyrinogen oxidase (PPO or PROTOX).

In some embodiments, genes useful for conferring herbicide resistance in plants can include genes that encode herbicide resistance proteins. In some embodiments, herbicide resistance proteins can include herbicide tolerant versions of the following: an acetyl coenzyme A carboxylase (ACCase); a 4-hydroxyphenylpyruvate dioxygenase (HPPD); an acetolactate synthase (ALS); a glutamine synthetase (GS); an 5-enolpyruvylshikimate-3-phosphate synthase (EPSPS); a protoporphyrinogen oxidase (PPO or PROTOX); an auxin enzyme or receptor; or any combination thereof. In some embodiments, herbicide resistance proteins can include herbicide inactivating enzymes such as the following: a glyphosate oxidoreductase (GOX); a glyphosate N-acetyltransferase (GAT); a phosphinothricin acetyl transferase (PAT); a dicamba monooxygenase; a P450 polypeptide; or any combination thereof.

In some embodiments, as used herein, “Hydroxyphenylpyruvate dioxygenase” and “HPPD”, “4-hydroxy phenyl pyruvate (or pyruvic acid) dioxygenase (4-HPPD)” and “p-hydroxy phenyl pyruvate (or pyruvic acid) dioxygenase (p-OHPP)” can be synonymous and can refer to a non-heme iron-dependent oxygenase that catalyzes the conversion of 4-hydroxyphenylpyruvate to homogentisate. In some embodiments, in organisms that degrade tyrosine, a reaction catalyzed by HPPD can be a second step in a pathway. In some embodiments, in plants, formation of homogentisate can be necessary for the synthesis of plastoquinone, which can serve as a redox cofactor, and tocopherol. In some embodiments, a polynucleotide molecule encoding an herbicide tolerant hydroxyphenylpyruvate dioxygenase (HPPD) can provide tolerance to HPPD inhibitors.

In some embodiments, as used herein, an “HPPD inhibitor” can comprise any compound or combinations of compounds which can decrease an ability of HPPD to catalyze a conversion of 4-hydroxyphenylpyruvate to homogentisate. In specific embodiments, an HPPD inhibitor can comprise an herbicidal inhibitor of HPPD. In some embodiments, non-limiting examples of HPPD inhibitors include, triketones (such as, mesotrione, sulcotrione, topramezone, and tembotrione); isoxazoles (such as, pyrasulfotole and isoxaflutole); pyrazoles (such as, benzofenap, pyrazoxyfen, and pyrazolynate); and benzobicyclon. In some embodiments, agriculturally acceptable salts of various inhibitors can include salts (e.g., cations or anions) for a formation of salts for agricultural or horticultural use.

In some embodiments, an “herbicide-resistant ALS polypeptide”, an “herbicide-tolerant ALS polypeptide” and an “ALS inhibitor-tolerant polypeptide” can be used interchangeably and can comprise any polypeptide which when expressed in a plant can confer tolerance to at least one acetolactate synthase (ALS) inhibitor. In some embodiments, ALS inhibitors can include, for example, sulfonylurea, imidazolinone, triazolopyrimidines, pryimidinyoxy(thio)benzoates, and/or sulfonylaminocarbonyltriazolinone herbicides. In some embodiments, ALS mutations can fall into different classes with regard to tolerance to, for example, sulfonylureas, imidazolinones, triazolopyrimidines, pyrimidinyl(thio)benzoates, and sulfonylaminocarbonyltriazolinone. In some embodiments, ALS mutations can include mutations having one or more of the following characteristics: (1) broad tolerance to all five of these groups (i.e., sulfonylureas, imidazolinones, triazolopyrimidines, pyrimidinyl(thio)benzoates, and sulfonylaminocarbonyltriazolinone); (2) tolerance to four of these groups (e.g., sulfonylureas, triazolopyrimidines, pyrimidinyl(thio)benzoates, and sulfonylaminocarbonyltriazolinone); (3) tolerance to imidazolinones and pyrimidinyl(thio)benzoates; (4) tolerance to sulfonylureas and triazolopyrimidines; and (5) tolerance to sulfonylureas and imidazolinones.

In some embodiments, the imidazolinone can include an imazapyr, an imazapic, an imazethapyr, an imazamox, an imazamethabenz, an imazaquin, a salt of any of these, a stereoisomer of any of these, or any combination thereofs. In some embodiments, the triazolopyrimidine can include a penoxsulam, a cloransulam-methyl, a diclosulam, a florasulam, a flumetsulam, a metosulam, a pyroxsulam, a salt of any of these, a stereoisomer of any of these, or any combination thereof. In some embodiments, the pyrimidinyl benzoate can include a bispyribac-sodium, a pyribenzoxim, a pyrithiobac-sodium, a salt of any of these, a stereoisomer of any of these, or any combination thereof. In some embodiments, the sulfonanilide can include a pyrimisulfan, a triafamone, a salt of any of these, a stereoisomer of any of these, or any combination thereof. In some embodiments, the sulfonylaminocarbonyltriazolinone can include a Flucarbazone-Na, a propoxycarbazone-Na, a thiencarbazone-methyl, a salt of any of these, a stereoisomer of any of these, or any combination thereof. In some embodiments, the sulfonylurea can include an amidosulfuron, an azimsulfuron, a bensulfuron-methyl, a chlorimuron-ethyl, a chlorsulfuron, a cinosulfuron, a cyclosulfamuron, an ethametsulfuron-methyl, an ethoxysulfuron, a flazasulfuron, a flucetosulfuron, a flupyrsulfuron-methyl-na, a foramsulfuron, a halosulfuron-methyl, an imazosulfuron, an iodosulfuron-methyl-na, a mesosulfuron-methyl, a metazosulfuron, a metsulfuron-methyl, a nicosulfuron, an orthosulfamuron, an oxasulfuron, a primisulfuron-methyl, a propyrisulfuron, a prosulfuron, a pyrazosulfuron-ethyl, a rimsulfuron, a sulfometuron-methyl, a sulfosulfuron, a thifensulfuron-methyl, a triasulfuron, a tribenuron-methyl, a trifloxysulfuron-na, a triflusulfuron-methyl, a tritosulfuron, a salt of any of these, a stereoisomer of any of these, or any combination thereof.

In some embodiments, polynucleotide molecules encoding proteins involved in herbicide resistance can include a polynucleotide molecule encoding an herbicide tolerant 5-enolpymvylshikimate-3-phosphate synthase (EPSPS) for example, for imparting glyphosate tolerance.

In some embodiments, glyphosate tolerance can also be obtained by expression of polynucleotide molecules encoding a glyphosate oxidoreductase (GOX) or a glyphosate-N-acetyl transferase (GAT).

In some embodiments, polynucleotides encoding a heterologous phosphinothricin acetyltransferase can be used for herbicide resistance. In some embodiments, plants containing a heterologous phosphinothricin acetyltransferase can exhibit improved tolerance to glufosinate herbicides, which can inhibit, for example, the enzyme glutamine synthetase.

In some embodiments, polynucleotides encoding proteins with altered protoporphyrinogen oxidase (PPO or PROTOX) activity can be used for herbicide resistance. In some embodiments, plants containing such polynucleotides can exhibit improved tolerance to any of a variety of herbicides which can target, for example, the PPO enzyme (also referred to as “PPO inhibitors” or “PROTOX inhibitors”).

In some embodiments, dicamba monooxygenase can be used for providing dicamba tolerance.

In some embodiments, a polynucleotide molecule encoding AAD12 or encoding AADI can be used for providing resistance to, for example, auxin herbicides.

In some embodiments, a P450-encoding polynucleotide can be used for conferring herbicide resistance. In some embodiments, a P450-encoding sequence can provide tolerance to HPPD inhibitors by, for example, metabolism of the herbicide. Such sequences include, but are not limited to, the NSF1 gene.

### Resistance to Plant Pests

In some embodiments, a “plant pest” can mean any living stage of an entity that can directly or indirectly injure, cause damage to, or cause disease in any plant or plant product. In some embodiments, a plant pest can include a protozoan, a nonhuman animal, a parasitic plant, a bacterium, a fungus, a virus, a viroid, an infectious agent, a pathogen, or any article similar to or allied thereof.

In some embodiments, a plant pest invertebrate can comprise a pest nematode, a pest mollusk, a pest insect, or any combination thereof. In some embodiments, a pest mollusk can comprise a slug, a snail, or a combination thereof. In some embodiments, a plant pathogen can comprise a fungus, a nematode, or a combination thereof.

In some embodiments, a plant pathogen can be a eukaryotic plant pathogen. In some embodiments, a plant pathogen can include, for example, a fungal pathogen, such as a phytopathogenic fungus.

In some embodiments, a target gene of interest (e.g., for gene silencing) can include any coding or non-coding sequence from any species (including, but not limited to, eukaryotes such as fungi; plants, including monocots and dicots, such as crop plants, ornamental plants, and non-domesticated or wild plants; invertebrates such as arthropods, annelids, nematodes, and mollusks; and vertebrates such as amphibians, fish, birds, and mammals). In some embodiments, non-limiting examples of a non-coding sequence (e.g., that can be expressed by a gene expression element such as a regulatory sequence) can include, 5′ untranslated regions, promoters, enhancers, or other non-coding transcriptional regions, 3′ untranslated regions, terminators, introns, microRNAs, microRNA precursor DNA sequences, small interfering RNAs, RNA components of ribosomes or ribozymes, small nucleolar RNAs, and other non-coding RNAs, or any combination thereof. In some embodiments, a gene of interest can include, translatable (coding) sequence, such as genes encoding transcription factors and genes encoding enzymes involved in a biosynthesis or catabolism of molecules of interest (such as amino acids, fatty acids and other lipids, sugars and other carbohydrates, biological polymers, and secondary metabolites including alkaloids, terpenoids, polyketides, non-ribosomal peptides, and secondary metabolites of mixed biosynthetic origin).

In some embodiments, a target gene (e.g., for gene silencing) can be an essential gene of a plant pest or plant pathogen. In some embodiments, essential genes can include genes that can be required for development of a pest or pathogen to a fertile reproductive adult. In some embodiments, essential genes can include genes that, when silenced or suppressed, can result in a death of an organism (e.g., as an adult or at any developmental stage, including gametes) or in an organism's inability to successfully reproduce (e. g., sterility in a male or female parent or lethality to a zygote, embryo, or larva).

In some embodiments, a plant can be transformed (e.g., in a nucleus, a cytoplasmic organelle, or both) with an expression cassette encoding, for example, a dsRNA, a siRNA or a miRNA. The dsRNA, siRNA, or miRNA can suppress (e.g., expression of) at least one (e.g., at least 1, at least 2, at least 3, at least 4, at least 5, at least 6, at least 7, at least 8, at least 9, or at least 10) target genes present in a plant pest. In some embodiments, a dsRNA, siRNA, or miRNA can suppress, for example, 1, 2, 3, 4, 5, 6, 7, 8, 9, 10, 15, 20 or more target genes of a plant pest. In some embodiments, suppression of a target gene present in a plant pest can provide complete or nearly complete protection from a plant pest. In some embodiments, “complete protection” can mean that no (e.g., substantial) damage can be caused to a plant by a plant pest.

In some embodiments, resistance to pests in plants can be achieved by, for example, transgenic control. In some embodiments, in-plant transgenic control of, for example, insect pests, can be achieved through, for example, plant expression of crystal (Cry) delta endotoxin genes and/or Vegetative Insecticidal Proteins (VIP) such as from Bacillus thuringiensis. In some embodiments, non-limiting examples of Cry toxins include, for example, the 60 main groups of “Cry” toxins (e.g., Cry1-Cry59) and VIP toxins. In some embodiments, cry toxins can include subgroups of Cry toxins, for example, Cry 1a.

In some embodiments, an expression cassette for use in transformation (e.g., into an organelle) may be constructed using, for example, a Cry sequence. In some embodiments, a Cry sequence can include, for example, a wild-type (e.g., native) nucleic acid sequence encoding at least one protein selected from a group consisting of: Cry1Ac, Cyt1Aa, Cry1Ab, Cry2Aa, Cry 1I, Cry1C, Cry1D, Cry1E, Cry1Be, Cry1Fa and Vip3A. In some embodiments, a Cry sequence can include, for example, a modified (e.g., truncated or fusion) nucleic acid sequence encoding at least one protein selected from a group consisting of: Cry1Ac, Cyt1Aa, Cry1Ab, Cry2Aa, Cry1I, Cry1C, Cry1D, Cry1E, Cry1Be, Cry1Fa and Vip3A. In some embodiments, a modified sequence can comprise a truncated nucleic acid sequence. In some embodiments, a modified sequence can encode a modified protein fragment. In some embodiments, a truncated protein fragment can retain insecticidal activity. In some embodiments, a nucleic acid sequence can encode a full-length, or modified (e.g., truncated) protein. In some embodiments, a modified protein can be codon-optimized for an organelle of interest.

### Methods for Delivery

In some embodiments, any suitable delivery method can be used for introducing a composition and molecule disclosure herein into a host cell or organelle. In some embodiments, an organelle can comprise a mitochondrion, a plastid, or a combination thereof. In some embodiments, a host cell can comprise a yeast cell, a plant cell, or a combination thereof. In some embodiments, a composition can comprise a site-directed nuclease, a donor polynucleotide, a nucleic acid encoding a composition, or any combination thereof. In some embodiments, a composition can be delivered simultaneously or temporally separated. In some embodiments, a choice of method of genetic modification can be dependent on a type of cell being transformed, a circumstance under which a transformation is taking place, or a combination thereof. In some embodiments, a circumstance under which a transformation is taking place can be in vitro, ex vivo, in vivo, in planta, or any combination thereof.

In some embodiments, a delivery method or transformation can include, an Agrobacterium-mediated transformation, a viral or bacteriophage infection, a transfection, a conjugation, a protoplast fusion, a lipofection, an electroporation, a calcium phosphate precipitation, a polyethyleneimine (PEI)-mediated transfection, a DEAE-dextran mediated transfection, a liposome-mediated transfection, a particle bombardment technology, a calcium phosphate precipitation, a direct micro injection, a nanoparticle-mediated nucleic acid delivery, a lipid nanoparticle, lipid-based vectors, polymeric vectors, polyethylenimine, poly(L-lysine), a vacuum infiltration, or any combination thereof.

In some embodiments, a DNA transformation can comprise a yeast nuclear genome transformation. In some embodiments, a DNA transformation can be facilitated by a development of shuttle vectors that can replicate in E. coli and yeast as autonomous plasmids. In some embodiments, a vector system can include low-copy-number plasmids and integrative DNA through homologous recombination.

In some embodiments, disclosed herein are methods comprising delivering a polynucleotide as described herein, a vector as described herein, a transcript thereof, a protein translated therefrom, or any combination thereof to a host cell or organelle. In some embodiments, disclosed herein is a cell produced by a method disclosed herein, an organism produced by a method disclosed herein, an organelle comprising or produced from a cell disclosed herein, or any combination thereof. In some embodiments, an organism can comprise an animal, a plant, a fungus, or a combination thereof.

In some embodiments, a method to introduce nucleic acids can comprise viral based gene transfer methods, non-viral based gene transfer methods, or a combination thereof. In some embodiments, a delivery vehicle can comprise a liposome. In some embodiments, a viral vector delivery system can include a DNA virus, an RNA virus, or a combination thereof. In some embodiments, a viral vector delivery system can have either episomal or integrated genomes after delivery to a cell. In some embodiments, a viral vector based system for gene transfer can comprise a retrovirus, a lentivirus, an adenovirus, an adeno-associated virus, a herpes simplex virus, or any combination thereof.

In some embodiments, an adenoviral-based system can be used. In some embodiments, an adenoviral-based system can lead to a transient expression of a transgene. In some embodiments, an adenoviral based vector can have a high transduction efficiency in cells and may not require cell division. In some embodiments, a high titer, high levels of expression, or a combination thereof can be obtained with an adenoviral based vector. In some embodiments, an adeno-associated virus (“AAV”) vector can be used to transduce a cell with a target nucleic acid. In some embodiments, a vector can be used to transduce a cell with a target nucleic acid for an in vitro production of nucleic acids and peptides, for in vivo and ex vivo gene therapy procedures, or any combination thereof.

In some embodiments, a cell transfected with one or more vectors described herein can be used to establish a new cell line comprising one or more vector-derived sequences. In some embodiments, a cell can be transiently transfected with a composition disclosed herein. In some embodiments, transient transfection can comprise transient transfection of one or more vectors, transfection with RNA, or a combination thereof. In some embodiments, a transiently transfected cell can be modified through an activity of a site-directed nuclease. In some embodiments, a cell modified through an activity of a site-directed nuclease can be used to establish a new cell line comprising cells containing a modification but lacking any other heterologous sequence.

In some embodiments, a composition as disclosed herein can be operably linked (e.g., covalently, or non-covalently) to a polypeptide permeant domain to promote uptake by a cell or an organelle. In some embodiments, a polynucleotide composition can comprise a DNA, an RNA, or a combination thereof. In some embodiments, a disclosure can be associated with a peptide-based polynucleotide carrier that can comprise two functional units: a polynucleotide-binding domain (e.g., a polycationic KH repeat domain) and a polypeptide permeant domain.

In some embodiments, a number of polypeptide permeant domains can be used in a non-integrating polypeptide as disclosed herein, including a peptide, a peptidomimetic, a non-peptide carrier, and any combination thereof. In some embodiments, the terms “permeant peptide”, “cell penetrating peptide”, “CPP”, “protein transduction domain” and “PTD” can be used interchangeably herein. In some embodiments, a permeant peptide can be derived from a third alpha helix of Drosophila melanogaster transcription factor Antennapaedia, referred to as penetratin. In some embodiments, a CPP can comprise an amino acid sequence as described in SEQ ID NOS: 10, 11, 12, 13, 14, 15, 16, 17, 18, 19, 20, 21, 22, 23, 24, 25, 26, or 27 of International Patent Application PCT/US22/80942, herein incorporated by reference. In some embodiments, a CPP can comprise an amino acid sequence having at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to SEQ ID NOS: 10, 11, 12, 13, 14, 15, 16, 17, 18, 19, 20, 21, 22, 23, 24, 25, 26, or 27 of International Patent Application PCT/US22/80942, herein incorporated by reference. In some embodiments, a permeant peptide can comprise an HIV-1 tat basic region amino acid sequence, which can include, for example, amino acids 49-57 of a naturally-occurring tat protein. In some embodiments, a permeant domain can include a poly-arginine motif. In some embodiments, a poly-arginine motif can comprise a region of amino acids 34-56 of an HIV-1 rev protein, a nona-arginine, an octa-arginine, or any combination thereof. In some embodiments, a nona-arginine (R9) sequence can be used. In some embodiments, other cell penetrating peptides can include: Pep-1, MPG, gamma-ZEIN, Transportan, MAP, Pept 1, Pept 2, IVV-14, Ig (v), Amphiphilic model peptide, pVEC, HRSV, Bp100 TAT2 or any combination thereof. In some embodiments, a composition as disclosed herein can be fused to a combination of a polypeptide permeant domain. In some embodiments, a site at which a fusion can be made can be selected in order to optimize a biological activity, secretion, or binding characteristics of a polypeptide.

In some embodiments, a polynucleotide composition can comprise a DNA, an RNA, or any combination thereof. In some embodiments, a polynucleotide composition disclosed herein can be associated with a peptide-based polynucleotide carrier that can comprise an organellar targeting signal. In some embodiments, for organelle-specific delivery, a peptide-based polynucleotide carrier can comprise two functional units: a polynucleotide-binding domain (e.g., a polycationic KH repeat domain) and an organelle-targeting peptide (e.g., a chloroplast transit peptide, a mitochondrial targeting peptide).

In some embodiments, where two or more different site-directed nucleases can be provided to a cell, site-directed nucleases can be provided simultaneously. In some embodiments, two or more different site-directed nucleases can be provided consecutively.

### Methods of Plant Growth

In some embodiments, a cell, a plant, a transgenic seed, a progeny plant, or a transgenic plant comprising one or more exogenous polynucleotides in an edited mitochondria genome or an edited plastid genome described herein may be grown in a temperature-controlled incubator, a bioreactor, a greenhouse, or a combination thereof. In some cases, the temperature-controlled incubator and/or greenhouse is further configured to control a light-dark cycle. In some embodiment, a cell, a plant, a transgenic seed, a progeny plant, or a transgenic plant can be grown in darkness for predetermined duration in predetermined temperature. In some embodiments, a cell, a plant, a transgenic seed, a progeny plant, or a transgenic plant can be grown in darkness for 16-20 hours at 26° C. In some embodiments, a plant, a transgenic seed, a progeny plant, or a transgenic plant can be grown in a continuous light growth environment at 26-28° C. for root and shoot formation. In some embodiments, a plant, a transgenic seed, a progeny plant, or a transgenic plant can be grown in a 16 h/8 h light/dark growth chamber at 26-28° C. for root and shoot formation. In some embodiments, a progeny plant or a transgenic plant showing both root and shoot development may be transferred to pots containing an artificial potting medium and gently acclimatized to greenhouse conditions. In some embodiments, a plant, a transgenic seed, a progeny plant, or a transgenic plant can be grown in a field. In some embodiments, a field may be treated with an herbicide, e.g., an inhibitor of acetolactate synthase.

### Compositions and Kits

Also provided herein are compositions that include any of the polynucleotides, polypeptides, vectors, or reagents described herein. Any of the compositions can include any of the polynucleotides, polypeptides, vectors, or reagents described herein and one or more (e.g., 1, 2, 3, 4, or 5) acceptable carriers or diluents. In some embodiments, the kit can include a cell, a tissue, a propagation material, a seed, a pollen, a progeny, or any combination thereof.

In some embodiments, any of the compositions described herein can include one or more buffers (e.g., a neutral-buffered saline, a phosphate-buffered saline (PBS)), one or more growth regulators (e.g., naphthaleneacetic acid, 6-benzylamino purine, phytagel), and one or more medium (e.g., germination medium, growth medium, maturation medium, phosphite medium).

In some embodiments, any of the compositions described herein can further include one or more (e.g., 1, 2, 3, 4, or 5) agents that promote the entry of any of the vectors or nucleic acids described herein into a cell (e.g., a plant cell).

In some embodiments, any of the vectors or nucleic acids described herein can be formulated using natural and/or synthetic polymers. Non-limiting examples of polymers that can be included in any of the pharmaceutical compositions described herein can include, but are not limited to: poloxamer, chitosan, dendrimers, and poly(lactic-co-glycolic acid) (PLGA) polymers.

Also provided are kits that include any of the compositions described herein that include any of the polynucleotides, any of the polypeptides, any of the reagents, or any of the vectors described herein.

In some embodiments, the kit can include instructions for performing any of the methods described herein.

In some embodiments, a nucleic acid sequence or amino acid sequence disclosed herein have at least about 10%, at least about 20%, at least about 30%, at least about 40%, at least about 50%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to a reference nucleic acid sequence or amino acid sequence disclosed herein. Furthermore, a method or composition disclosed herein can use the appropriate nucleic acid or amino acid or portion thereof as needed. In some embodiments, the composition disclosed herein can be provided in a kit with instructions for use.

The section headings used herein are for organizational purposes only and are not to be construed as limiting the subject matter described.

### EXAMPLES

The present disclosure is further defined in the following Examples, in which parts and percentages are by weight and degrees are Celsius, unless otherwise stated. It should be understood that these Examples, while indicating embodiments, are given by way of illustration only. From the above discussion and these Examples, the essential characteristics of this disclosure can be ascertained, and without departing from the spirit and scope thereof, various changes and modifications of the disclosure can be envisioned to adapt it to various usages and conditions. Such modifications are also intended to fall within the scope of the appended claims.

### Example 1: Use of TALENs to Enhance Replacement of a Wild-Type Mitochondrial DNA Region with a Donor DNA

Wild-type callus cells were induced from the embryos of mature seed of rice, Oryza sativa L. ssp. Japonica cv. Nipponbare, and were co-transformed with a Donor DNA fragment of the mitochondrial-expression construct pNAP433 and a plasmid DNA of the nuclear-expression construct pNAP159 by use of the biolistic method. The Donor DNA (SEQ ID NO: 26) of plasmid pNAP433 contained an mALS (herbicide resistance-large subunit, HR-LS) coding region (with a rice mitochondrial cox2 RNA editing site; SEQ ID NO: 19) operably linked to a hybrid T7+rice ATP1 promoter (SEQ ID NO: 27) and a truncated version of the hybrid T7+rice ATP1 terminator (SEQ ID NO: 28). The mitochondrial cox2 RNA editing site was included to produce a targeted C-to-U conversion to create the translation start codon, AUG from ACG, in the corresponding mRNA. The Donor DNA fragment also contained a nucleotide sequence (SEQ ID NO: 29) encoding the orf79 protein (SEQ ID NO: 8) and a gRNA cassette (SEQ ID NO: 30) for potential use with a MAD7 nuclease (SEQ ID NO: 31). The Donor DNA also had rice mitochondrial DNA homologous regions at the 5′ and 3′ ends of 1.6 kb (SEQ ID NO: 32) and 1.2 kb (SEQ ID NO: 33), respectively.

Plasmid pNAP159 had the following two nuclear expression cassettes: ZmUbi1 promoter::MTS-T7 Polymerase::NOS terminator; and CaMV 35S promoter::HPT::CaMV 35S terminator. The nucleotide sequence encoding the MTS-T7 RNA polymerase is presented as SEQ ID NO: 34. The corresponding amino acid sequence of the MTS-T7 RNA polymerase is presented as SEQ ID NO: 35. The MTS amino acid sequence (SEQ ID NO: 36) used for the MTS-T7 RNA polymerase was from the Arabidopsis thaliana At5g47030 gene.

Using the mALS (HR-LS) and HPT genes as positive selectable markers for chlorsulfuron and hygromycin resistance, respectively, mitochondrial transformants were selected as described in International Patent Application Publication No. WO2024187111A2 (Published Sep. 12, 2024). The events were further screened for the correct integration of the Donor DNA at the targeted mitochondrial genome site, i.e., downstream of the mitochondrial atp6 gene. To confirm the correct integration of Donor DNA into the mitochondrial genome, PCR analyses of the junction regions were performed. Each junction region was amplified with a primer specific to the Donor DNA region and another primer specific to mitochondrial genomic sequence in the vicinity of the homologous region of Donor DNA. Multiple events were identified by this analysis (International Patent Application Publication No. WO2024187111A2). The correct integration of positive events was also confirmed by sequencing of the junction PCR fragments.

The mtDNA carrying the Donor DNA insertion was found in a minor percentage of the entire mtDNA population as evident from the PCR analysis described above. In the analysis, the PCR junction fragments were detectable after more than 35 cycles of PCR reactions using total cell DNA as a template. Using the same total cell DNA, a wild-type mtDNA gene, e.g., the cox2 gene, could be amplified by PCR reactions with fewer than 30 cycles. This indicated that the proportion of mtDNA molecules carrying the Donor DNA was in the range of a few percentages of the total mtDNA molecules in callus cells even after several months of chlorsulfuron selection for the mALS (HR-LS) marker gene carried on the Donor DNA. This was also confirmed by deep sequencing of polymorphisms present in the homologous region of Donor and wild-type mitochondrial DNA.

To achieve further enrichment of engineered mtDNA containing the Donor DNA insertion, which is critical for stable transmission of the engineered mtDNA over cell divisions and generations without selection, a new method was implemented to reduce or eliminate wild-type DNA (e.g., by at least about 10%, at least about 20%, at least about 30%, at least about 40%, at least about 50%, at least about 60%, at least about 70%, at least about 80%, at least about 90%, or at least about 95% as compared to untreated cells. in the transformed plant cells. For this purpose, we introduced into the transformed callus tissue nuclear-expression units encoding site-directed nucleases (SDNs) that cleave wild-type mtDNA, but not the engineered mtDNA. In this Example, each plasmid encoded a pair of mitochondrially targeted TAL-effector nucleases (TALENs) that were designed to recognize wild-type mtDNA sequences that were not present in the engineered mtDNA containing the Donor DNA, i.e., the region of the wild-type mtDNA that was replaced in the engineered mtDNA. The wild-type mtDNA region replaced by the pNAP433 Donor DNA was a 505 bp-long region (SEQ ID NO: 37) downstream of the mitochondrial atp6 gene.

A pair of TALENs were designed to recognize mitochondrial sequences that were separated by 12 bp, with the two TALEN recognition sites present on complementary DNA strands. One member of a TALEN pair, TAL-C, targeted the following 18-mer sequence of the wild-type mitochondrial DNA:

(SEQ ID NO: 38)
CTCATCGCCAACAAAAGG

The corresponding other member of the TALEN pair, TAL-D, targeted the following 18-mer sequence:

(SEQ ID NO: 39)
CCATAGTACATGCCGATT

The coding sequences for the TAL-C and TAL-D TALENs were synthesized in the form of mRNA by the vendor, Thermo Fisher Scientific, Inc. The TALEN sequences encoded in each mRNA were converted into the corresponding DNA by reverse transcriptase reactions and the DNA fragments were cloned into a Gateway vector.

Subsequently, each TALEN ORF was modified to have a mitochondrial targeting sequence (MTS) instead of a nuclear targeting sequence (NLS), and the sequences of constant regions were altered to avoid internal homologous recombination. The resulting ORFs were each operably linked to a promoter and a terminator sequence for expression in the nucleus. The resulting two TALEN expression units, encoding MTS-TAL-C(SEQ ID NO: 41) and MTS-TAL-D (SEQ ID NO: 44), were cloned into a binary construct carrying a selectable marker gene for nuclear transformation encoding MTS(AT5G47030)-Sul1 (SEQ ID NO: 46), the Sul1 protein (SEQ ID NO: 47) with the mitochondrial targeting sequence (SEQ ID NO: 36) from AT5G47030, which confers resistance to the sulfonamide, sulfadiazine (SDZ). The binary vector also contained an expression unit for producing MTS(rps10)-folk (SEQ ID NO: 49), the folk protein (SEQ ID NO: 2) with the mitochondrial targeting sequence (SEQ ID NO: 42) from rice rps10.

The resulting T-DNA construct, pNAP544, was in the pCAMBIA0380 vector and had the following expression cassettes in the region transferred to the nuclear genome:
OsTubA1 promoter::MTS(rps10)-TAL-C::HSP18.2 terminator (SEQ ID NO: 50);
ZmUbi1 promoter::MTS(AT5G47030)-TAL-D::OCS terminator (SEQ ID NO: 51);
OsActin1 promoter::MTS(AT5G47030)-Sul1::NOS terminator (SEQ ID NO: 52); and
CaMV 35S promoter::MTS(rps10)-folK::CaMV 35S terminator (SEQ ID NO: 53).

To eliminate wild-type mtDNA in the events transformed with mitochondrial Donor DNA, selected mitochondrial transgenic events were re-transformed with the Agrobacterium strain, AGL-1, carrying the T-DNA construct, pNAP544. Agrobacterium-transformed cells underwent selection (e.g., for at least about 2 weeks, at least about 4 weeks, at least about 6 weeks, at least about 8 weeks, at least about 3 months, at least about 4 months, at least about 5 months or at least about 6 months) on medium containing both chlorsulfuron and sulfadiazine. The events obtained by this selection were subjected to PCR analyses to reconfirm the presence of the integrated Donor DNA as well as the enrichment of the engineered mtDNA in the cellular pool of mitochondrial genomes.

The assessment of the replacement of wild-type mtDNA with engineered mtDNA was performed by a duplex PCR analysis as follows. One pair of primers (Donor-3 and Donor-4), specific to the Donor DNA, had the following sequences:

Donor-3:
(SEQ ID NO: 54)
GCTACTGGTGTTGGGCAACATCAA
Donor-4:
(SEQ ID NO: 55)
ATATGTATGCGCCCTATTCGCCTTA

A second pair of primers (WT-2 and WT-3), specific to the wild-type mtDNA replaced by the Donor DNA, had the following sequences:

WT-2:
(SEQ ID NO: 56)
TCGGTAATCCGAAGCCCACC
WT-3:
(SEQ ID NO: 57)
CCATAGTACATGCCGATTGAAGGATG

All primers had similar Tₘ temperatures of around 61° C. to allow for similar annealing efficiency in the duplex PCR reactions. Each duplex PCR reaction was set up with the following: 10 pmol of each of the four primers; One-Taq® DNA polymerase (New England Biolabs, Inc.); and total cell DNA from a callus sample (approximately 100 ng). The duplex PCR assay conditions were as follows: 94° C. for 30 sec, 94° C. for 20 sec, 54° C. for 30 sec, 68° C. for 30 sec (repeat 30 or 32 cycles to step 2), 68° C. for 5 min. PCR products were then separated on a 1.5% agarose gel.

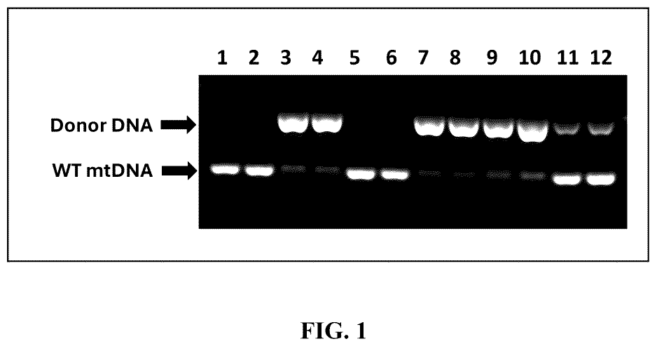

FIG. 1 shows a duplex PCR analysis of the regions specific to Donor DNA and to wild-type rice mitochondrial DNA. Two independent PCR reactions were performed for each sample. Wild-type rice DNA (lanes 1, 2, 5, and 6) was compared to DNA from events AA1 (lanes 3 & 4); AA5 (lanes 7 & 8). AA9 (lanes 9 & 10) and C97 (lanes 11 & 12).

Gel images were analyzed using ImageJ, a Java-based image processing program developed at the National Institutes of Health and the Laboratory for Optical and Computational Instrumentation (LOCI, University of Wisconsin).

The background intensity was subtracted from the intensity of each band. Since a larger DNA fragment shows a stronger intensity than a smaller DNA fragment due to randomly intercalated ethidium bromide molecules into the DNA, the intensity was normalized by multiplying the wild-type intensity value by 1.55 (318 bp/205 bp), so that the intensity of the wild-type band reflects the intensity of a band with the size same as the Donor DNA band. Accordingly, the following formula was used to calculate the percent replacement of wild-type mtDNA with gene-edited mtDNA:

$$Replacement \% = [ ( Intensity of Donor DNA band ) / [ ( Intensity of Donor DNA band ) + ( 1.55 ) ( Intensity of wild ‐ type DNA band ) ] ] ⨯ 100 \%$$

The quantitative analysis of the gel image shown in FIG. 1 is presented in Table 1.

TABLE 1
Replacement % of wild-type mtDNA with a donor
DNA after TALENs (e.g., TALENs comprising
FokI endonuclease domain) transformation

| | | Donor | WT | | Average |
|---|---|---|---|---|---|
| | | DNA* | DNA* | Replace- | Replace- |
| Lane | Sample | (318 bp) | (205 bp) | ment %** | ment % |
| 1 | WT-1 | 0.062 | 93.51 | 0.04276 | 0.3693 |
| 2 | WT-2 | 1.112 | 102.4 | 0.6958 | |
| 3 | AA1-1 | 163.4 | 15.63 | 87.09 | 87.51 |
| 4 | AA1-2 | 157.3 | 13.93 | 87.93 | |
| 5 | WT-2 | 1.604 | 107.3 | 0.9548 | 0.9048 |
| 6 | WT-2 | 1.239 | 92.71 | 0.8548 | |
| 7 | AA5-1 | 149.7 | 9.188 | 91.31 | 91.86 |
| 8 | AA5-2 | 143.5 | 7.602 | 92.41 | |
| 9 | AA9-1 | 146.3 | 12.46 | 88.34 | 86.32 |
| 10 | AA9-1 | 167.8 | 20.16 | 84.31 | |
| 11 | C97 | 37.38 | 104.0 | 18.82 | 19.19 |
| 12 | C97 | 43.35 | 115.0 | 19.56 | |

*Net intensity of band after subtraction of background intensity.
**Replacement % as calculated by equation given above.

The percent replacement of wild-type mtDNA with Donor DNA of three independent events after TALEN transformation (AA1, AA5 and AA9) was 88%, 92% and 86%, respectively, while the founder line of these events (C97) exhibited 19% replacement after continuous cultivation without TALEN transformation. Other independent events derived from TALEN transformation of the founder event C97 also gave comparable values for Donor DNA replacement of wild-type mtDNA (AA2: 85%; AA3: 90%; and AA7: 93%).

The percent of Donor DNA within the entire mtDNA cellular pool was also analyzed by determining the frequency of a polymorphic allele present in the 3′ HR. After deep sequencing of PCR products, the AA2 event showed 97.0% replacement (22,755 reads of Donor polymorphism and 695 reads of wild-type), the AA3 event showed 96.6% replacement (21,268 reads of Donor polymorphism and 755 reads of wild-type), and the AA7 event showed 97.6% replacement (22,255 reads of Donor polymorphism and 548 reads of wild-type). Given the fact that the rice nuclear genome contained endogenous regions of mtDNA sequence, which would be expected to be represented in the duplex PCR and deep sequencing analyses, the events created after TAL transformation were at or near homoplasmy.

### Example 2: Use of Novel TALENs to Enhance Replacement of a Wild-Type Mitochondrial DNA Region with a Donor DNA

Wild-type callus cells were induced from the embryos of mature seed of rice, Oryza sativa L. ssp. Japonica cv. Nipponbare, and were co-transformed with the Donor DNA fragment (SEQ ID NO: 58) of the mitochondrial-expression construct pNAP432 and the plasmid DNA of the nuclear-expression construct pNAP195 by use of the biolistic method. The Donor DNA of plasmid pNAP432 is identical to the Donor DNA fragment of pNAP433 as described in Example 1, with the exception that the mALS (HR-LS) coding region comprises a rice mitochondrial nad4L RNA editing site (SEQ ID NO: 18) instead of a cox2 RNA editing site (SEQ ID NO: 19). The nad4L RNA editing site was included to produce a targeted C-to-U conversion to create the translation start codon, AUG from ACG, in the corresponding mRNA. Plasmid pNAP195 has the following three nuclear expression cassettes: ZmUbi1 promoter::MTS-T7 Polymerase::OCS terminator; OsActin1 promoter: MTS-ALS (SS)::NOS terminator; and CaMV 35S promoter::HPT::CaMV 35S terminator. The pNAP195 plasmid is similar to pNAP159, but also has an MTS-ALS (SS) expression unit. SEQ ID NO: 59 corresponds to the nucleotide sequence encoding the MTS-ALS (SS) fusion protein. SEQ ID NO: 13 corresponds to the amino acid sequence of the MTS-ALS (SS) fusion protein encoded by SEQ ID NO: 59. The 36 amino acid MTS sequence (SEQ ID NO: 36) used for the MTS-ALS (SS) fusion protein was from the Arabidopsis thaliana At5g47030 gene.

Using the mALS (HR-LS) and HPT genes as positive selectable markers for chlorsulfuron and hygromycin resistance, respectively, mitochondrial transformants were selected as described in International Patent Application Publication No. WO2024187111A2 (Published Sep. 12, 2024). The events were further screened for the correct integration of the Donor DNA at the targeted mitochondrial genome site, i.e., downstream of the mitochondrial atp6 gene. To confirm the correct integration of Donor DNA into the mitochondrial genome, PCR analyses of the junction regions were performed. Each junction region was amplified with a primer specific to the Donor DNA region and another primer specific to mitochondrial genomic sequence in the vicinity of the homologous region of Donor DNA. Multiple events were identified by this analysis (International Patent Application Publication No. WO2024187111A2). The correct integration of positive events was also confirmed by sequencing of the junction PCR fragments.

In this example, we made nuclear-expression constructs encoding a pair of novel mitochondrially targeted TAL-effector nucleases (TAL) that contained an alternate nuclease domain, the NAP12 nuclease domain (SEQ ID NO: 5), in place of the FokI nuclease domain (SEQ ID NO: 3). The NAP12 nuclease domain was derived from a TypeIIS restriction endonuclease from Lysobacter (GenBank Accession No. WP_056305938.1; SEQ ID NO: 4). The NAP12 nuclease domain fragment (SEQ ID NO: 60) contains both the amino-terminal nuclease domain of NAP12 (SEQ ID NO: 5) and the linker region (SEQ ID NO: 61) between the nuclease domain the carboxy-terminal DNA-binding domain.

As in the previous Example, we designed a pair of TALENs (e.g., TALENs comprising the NAP12 nuclease domain) to target the wild-type mtDNA region replaced by the pNAP432 Donor DNA, which was a 505 bp-long region (SEQ ID NO: 37) downstream of the mitochondrial atp6 gene. This pair of TALENs was designed to recognize mitochondrial DNA sequences that were separated by 15 bp and were coded on each of complementary DNA strands. One member of the TALEN pair, TAL-A1, targeted the following 18-mer sequence of the wild-type mitochondrial DNA:

(SEQ ID NO: 99)
TCGGTAATCCGAAGCCCA

The corresponding other member of the TALEN pair, TAL-B1, targeted the following 18-mer sequence:

(SEQ ID NO: 100)
GCAAGATCCTCAAGAAAA

The coding sequences for the corresponding TALENs were synthesized in the form of mRNA by the vendor, Thermo Fisher Scientific, Inc. The TALEN sequences encoded in each mRNA were converted into the corresponding DNA by reverse transcriptase reactions and the DNA fragments were cloned into a Gateway vector.

Each TALEN ORF was modified to have a mitochondrial targeting sequence (MTS) instead of a nuclear targeting sequence (NLS), and the sequences of constant regions were altered to avoid internal homologous recombination. The resulting ORFs were each operably linked to a promoter and a terminator sequence for expression in the nucleus. Additionally, the FokI nuclease domain of each TALEN was replaced with the NAP12 nuclease domain, to yield the following pair of TALEN proteins: MTS-TAL-A1-NAP12 (SEQ ID NO: 102) and MTS-TAL-B1-NAP12 (SEQ ID NO: 105). The corresponding pair of TALEN expression units was cloned into a binary construct carrying a selectable marker for nuclear transformation encoding MTS(AT5G47030)-Sul1 (SEQ ID NO: 46), the Sul1 protein with the mitochondrial targeting sequence from AT5G47030, which confers resistance to the sulfonamide, sulfadiazine (SDZ). The binary vector also contained an expression unit for producing MTS(rps10)-folk (SEQ ID NO: 49) the folk protein with the mitochondrial targeting sequence from rice rps10.

The resulting T-DNA construct, pNAP651, had the following expression cassettes in the region transferred to the nuclear genome:
OsTubA1 promoter::MTS(rps10)-TAL-A1-NAP12::HSP18.2 terminator (SEQ ID NO: 103);
ZmUbi1 promoter::MTS(AT5G47030)-TAL-B1-NAP12::OCS terminator (SEQ ID NO: 106);
OsActin1 promoter::MTS(AT5G47030)-Sul1::NOS terminator (SEQ ID NO: 52); and
CaMV 35S promoter::MTS(rps10)-folK::CaMV 35S terminator (SEQ ID NO: 53).

To eliminate wild-type mtDNA in the events transformed with mitochondrial Donor DNA, selected mitochondrial transgenic events were re-transformed with the Agrobacterium strain, AGL-1, carrying the T-DNA construct, pNAP651. Agrobacterium-transformed cells underwent selection (e.g., for at least about 2 weeks, at least about 4 weeks, at least about 6 weeks, at least about 8 weeks, at least about 3 months, at least about 4 months, at least about 5 months or at least about 6 months) on medium containing both chlorsulfuron and sulfadiazine. The events obtained by this selection were subjected to PCR analyses to reconfirm the presence of the integrated Donor DNA as well as the enrichment of the engineered mtDNA in the cellular pool of mitochondrial genomes.

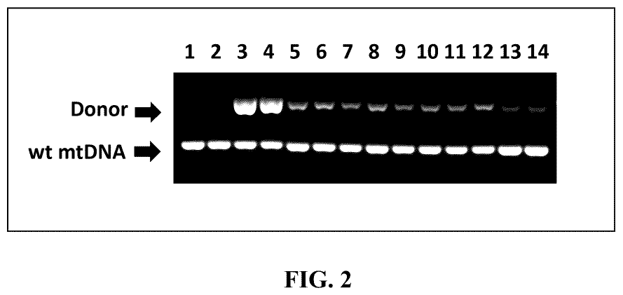

A regenerated plantlet from one re-transformation event, HH43, which was derived from Agrobacterium transformation of the parent C75 line with pNAP651, was selected for enrichment analysis by duplex PCR as described in the previous Example. FIG. 2 shows the duplex PCR analysis of regions specific to Donor DNA and to wild-type mitochondrial DNA. Wild-type rice DNA (lanes 1 & 2) is compared to leaf DNA from the TALENs transgenic event, HH43 (lanes 3 & 4), and to callus DNA from the following other transgenic events that did not contain TALENs expression units: GG61 (lanes 5 & 6); GG62 (lanes 7 & 8); GG63 (lanes 9 & 10); GG65 (lanes 11 & 12); and C75 (lanes 13 & 14).

The quantitative analysis of the gel image shown in FIG. 2 is presented in Table 2.

TABLE 2
Replacement % of wild-type mtDNA with a donor
DNA after TALENs (e.g., TALENs comprising
NAP12 endonuclease domain) transformation

| | | Donor | WT | | Average |
|---|---|---|---|---|---|
| | | DNA* | DNA* | Replace- | Replace- |
| Lane | Sample | (318 bp) | (205 bp) | ment %** | ment % |
| 1 | WT-1 | 0 | 87.41 | 0 | 0.1729 |
| 2 | WT-2 | 0.468 | 86.99 | 0.3459 | |
| 3 | HH43Leaf-1 | 131.9 | 81.08 | 51.21 | 51.30 |
| 4 | HH43Leaf-2 | 127.1 | 77.57 | 51.40 | |
| 5 | GG61-1 | 33.78 | 88.21 | 19.81 | 19.95 |
| 6 | GG61-2 | 32.94 | 84.59 | 20.08 | |
| 7 | GG62-1 | 21.47 | 80.10 | 14.74 | 16.53 |
| 8 | GG62-2 | 31.31 | 90.05 | 18.32 | |
| 9 | GG63-1 | 18.32 | 81.71 | 12.64 | 13.75 |
| 10 | GG63-2 | 23.02 | 85.08 | 14.86 | |
| 11 | GG65-1 | 20.21 | 78.49 | 14.25 | 15.29 |
| 12 | GG65-2 | 24.59 | 81.20 | 16.34 | |
| 13 | C75-1 | 8.774 | 103.2 | 5.198 | 5.237 |
| 14 | C75-2 | 9.101 | 105.4 | 5.276 | |

*Net intensity of band after subtraction of background intensity.
**Replacement % as calculated by equation given in previous Example.

The percent replacement of wild-type mtDNA with Donor DNA in leaf tissue of a regenerated plantlet from the HH43 event was 51%. The parent mitochondrial transformed event, C75, had 5% of engineered mtDNA prior to Agrobacterium re-transformation. Other events that were not re-transformed with pNAP651 but had only been selected for SU resistance (GG events in FIG. 2), had percent replacement values of 13-19% by the duplex PCR analysis of callus tissue.

We designed another pair of TALENs (e.g., TALENs comprising the NAP12 nuclease domain) to target the wild-type mtDNA region replaced by the pNAP432 Donor DNA, which was a 505 bp-long region (SEQ ID NO: 37) downstream of the mitochondrial atp6 gene. This pair of TALENs was designed to recognize mitochondrial DNA sequences that were separated by 17 bp and were coded on each of complementary DNA strands. One member of the TALEN pair, TAL-C1, targeted the following 18-mer sequence of the wild-type mitochondrial DNA:

(SEQ ID NO: 62)
GCAGCCTCATCGCCAACA

The corresponding other member of the TALEN pair, TAL-D, targeted the following 18-mer sequence:

(SEQ ID NO: 39)
CCATAGTACATGCCGATT

The coding sequences for the corresponding TALENs were synthesized in the form of mRNA by the vendor, Thermo Fisher Scientific, Inc. The TALEN sequences encoded in each mRNA were converted into the corresponding DNA by reverse transcriptase reactions and the DNA fragments were cloned into a Gateway vector.

Each TALEN ORF was modified to have a mitochondrial targeting sequence (MTS) instead of a nuclear targeting sequence (NLS), and the sequences of constant regions were altered to avoid internal homologous recombination. The resulting ORFs were each operably linked to a promoter and a terminator sequence for expression in the nucleus. Additionally, the FokI nuclease domain of each TALEN was replaced with the NAP12 nuclease domain, to yield the following pair of TALEN proteins: MTS-TAL-C1-NAP12 (SEQ ID NO: 64) and MTS-TAL-D-NAP12 (SEQ ID NO: 66). The corresponding pair of TALEN expression units was cloned into a binary construct carrying a selectable marker for nuclear transformation encoding MTS(AT5G47030)-Sul1 (SEQ ID NO: 46), the Sul1 protein with the mitochondrial targeting sequence from AT5G47030, which confers resistance to the sulfonamide, sulfadiazine (SDZ). The binary vector also contained an expression unit for producing MTS(rps10)-folk (SEQ ID NO: 49) the folk protein with the mitochondrial targeting sequence from rice rps10.

The resulting T-DNA construct, pNAP913, had the following expression cassettes in the region transferred to the nuclear genome:
OsTubA1 promoter::MTS(rps10)-TAL-C1-NAP12::HSP18.2 terminator (SEQ ID NO: 67);
ZmUbi1 promoter::MTS(AT5G47030)-TAL-D-NAP12::OCS terminator (SEQ ID NO: 68);
OsActin1 promoter::MTS(AT5G47030)-Sul1::NOS terminator (SEQ ID NO: 52); and CaMV 35S promoter::MTS(rps10)-folk::CaMV 35S terminator (SEQ ID NO: 53).

To eliminate wild-type mtDNA in the events transformed with mitochondrial Donor DNA, selected mitochondrial transgenic events are re-transformed with the Agrobacterium strain, AGL-1, carrying the T-DNA construct, pNAP913. Agrobacterium-transformed cells undergo selection (e.g., for at least about 2 weeks, at least about 4 weeks, at least about 6 weeks, at least about 8 weeks, at least about 3 months, at least about 4 months, at least about 5 months or at least about 6 months) on medium containing both chlorsulfuron and sulfadiazine. The events obtained by this selection are subjected to PCR analyses to reconfirm the presence of the integrated Donor DNA as well as the enrichment of the engineered mtDNA in the cellular pool of mitochondrial genomes.

### Example 3: Replacement of a Wild-Type Plastid DNA Region with Donor DNA

Wild-type callus cells were induced from the embryos of mature seed of rice, Oryza sativa L. ssp. Japonica cv. Nipponbare, and were co-transformed with the Donor DNA fragment of the plastid-expression construct pNAP670 and the plasmid DNA of the nuclear-expression construct pNAP199 by use of the biolistic method. The Donor DNA fragment (SEQ ID NO: 69) of plasmid pNAP670 contained an mALS (HR-LS) coding region operably linked to a rice plastid 16SrRNA promoter (SEQ ID NO: 70), a rice plastid psbA 5′-UTR (SEQ ID NO: 71), and a rice plastid psbA terminator (SEQ ID NO: 72). The Donor DNA had rice plastid DNA homologous regions at the 5′ and 3′ ends of 2.0 kb (SEQ ID NO: 73) and 2.0 kb (SEQ ID NO: 74), respectively. Plasmid pNAP199 had the following nuclear expression cassette: CaMV 35S promoter::HPT::CaMV 35S terminator (SEQ ID NO: 75). Using the mALS (HR-LS) and HPT genes as positive selectable markers for chlorsulfuron and hygromycin resistance, respectively, plastid transformants were selected as described for mitochondrial transformants in International Patent Application Publication No. WO2024187111A2 (Published Sep. 12, 2024). The events were further screened for the correct integration of the Donor DNA at the targeted plastid genome site, i.e., upstream of the plastid rpoB gene. To confirm the correct integration of Donor DNA into the plastid genome, PCR analyses of the junction regions were performed. Each junction region was amplified with a primer specific to the Donor DNA region and another primer specific to plastid genomic sequence in the vicinity of the homologous region of Donor DNA. The correct integration of a positive event was also confirmed by sequencing of the junction PCR fragments.

Primers rpoB_rev_3 (SEQ ID NO: 76) and us_ALS_rev_1 (SEQ ID NO: 77) were used to amplify a 2237-bp 5′ junction fragment corresponding to the 5′ region of Donor DNA insertion. Primers pED10_fwd_2 (SEQ ID NO: 78) and ALS_D626_for (SEQ ID NO: 79) were used to amplify a 2207-bp 3′ junction fragment corresponding to the 3′ region of Donor DNA insertion. For PCR analyses, total DNA from callus of each herbicide resistant event (5-20 mg) was isolated and resuspended in 30 μl TE. The DNA yield was about 420 ng/μl on average. 1 μl of DNA was used for each PCR reaction. The PCR reaction was prepared as follows: 1 μl of total DNA, 100 μmol of each primer and 10 μl LongAmp Taq 2× Master Mix (New England Labs Inc.) in a 20 μl reaction mix. The PCR reaction for the 5′ junction amplification was performed as follows: 2 min at 94° C.; then 40 cycles of 15 sec at 94° C., 30 sec at 59° C., and 2 min 10 sec at 65° C.; followed by the final incubation for ten minutes at 65° C. The PCR reaction for the 3′ junction amplification was performed as follows: 2 min at 94° C.; then 40 cycles of 15 sec at 94° C., 30 sec at 59° C., and 2 min 30 sec at 65° C.; followed by the final incubation for ten minutes at 65° C. The PCR samples were separated on 0.7% agarose gels.

SEQ ID NO: 80 shows a 160-bp region of the 2237-bp 5′ junction fragment that has 80-bp of unique plastid DNA sequence and 80-bp of 5′-HR DNA sequence that is present in both the Donor DNA and the wild-type plastid DNA. SEQ ID NO: 81 shows a 129-bp region of the 2237-bp 5′ junction fragment that has 80-bp of 5′-HR DNA that is present in both the Donor DNA and the wild-type plastid DNA and 49-bp of unique Donor DNA sequence. The 5′-junction of the Donor DNA integration is thereby confirmed.

SEQ ID NO: 82 shows a 133-bp region of the 2207-bp 3′ junction fragment that has 80-bp of unique Donor DNA sequence and 53-bp of 3′-HR DNA sequence that is present in both the Donor DNA and wild-type plastid DNA. SEQ ID NO: 83 shows a 109-bp region of the 2207-bp 3′ junction fragment that has 80-bp of 3′-HR DNA sequence that is present in both Donor DNA and wild-type plastid DNA and 29-bp of unique plastid DNA sequence. The 3′-junction of the Donor DNA integration is thereby confirmed.

### Example 4: Construction of TALENs to Enhance Replacement of a Wild-Type Plastid DNA Region with Donor DNA

A pair of TALENs were designed to recognize plastid sequences that were separated by 16 bp, with the two TALEN recognition sites present on complementary DNA strands. One member of a TALEN pair, Chl-TAL3, targeted the following 18-mer sequence of the rice wild-type plastid DNA:

(SEQ ID NO: 84)
CCGAACAGCATTATTATC

The corresponding other member of the TALEN pair, Chl-TAL4, targeted the following 18-mer sequence:

(SEQ ID NO: 85)
CTCCACAGAGATATGTAC

The coding sequences for the corresponding TALENs were synthesized in the form of mRNA by the vendor, Thermo Fisher Scientific, Inc. The TALEN sequences encoded in each mRNA were converted into the corresponding DNA by reverse transcriptase reactions and the DNA fragments were cloned into a Gateway vector.

Subsequently, each TALEN ORF was modified to have a chloroplast (plastid) targeting sequence (CTS) instead of a nuclear targeting sequence (NLS), and the sequences of constant regions were altered to avoid internal homologous recombination. Additionally, the FokI nuclease domain of each TALEN was replaced with the NAP12 nuclease domain. The resulting ORFs were each operably linked to a promoter and a terminator sequence for expression in the nucleus. The resulting two TALEN expression units, encoding CTS-Chl-TAL3 (SEQ ID NO: 87) and CTS-Chl-TAL4 (SEQ ID NO: 90), were cloned into a binary construct carrying a selectable marker gene for nuclear transformation encoding MTS(AT5G47030)-Sul1 (SEQ ID NO: 46) the Sul1 protein with the mitochondrial targeting sequence from AT5G47030, which confers resistance to the sulfonamide, sulfadiazine (SDZ). The binary vector also contained an expression unit for producing MTS(rps10)-folk (SEQ ID NO: 49), the folk protein with the mitochondrial targeting sequence from rice rps10.

The resulting T-DNA construct in pCAMBIA0380 vector, pNAP739, had the following expression cassettes in the region transferred to the nuclear genome:
OsTubA1 promoter::CTS (rbcS)-Chl-TAL3::HSP18.2 terminator (SEQ ID NO: 91);
ZmUbi1 promoter::CTS (rbcS)-Chl-TAL4::OCS terminator (SEQ ID NO: 92);
OsActin1 promoter::MTS(AT5G47030)-Sul1::NOS terminator (SEQ ID NO: 52);
CaMV 35S promoter::MTS(rps10)-folK::CaMV 35S terminator (SEQ ID NO: 53).

### Example 5: Use of TALENs to Enhance Replacement of a Wild-Type Plastid DNA Region with a Donor DNA

To eliminate wild-type plastid DNA in the events transformed with plastid Donor DNA, selected plastid transgenic events are re-transformed with the Agrobacterium strain, AGL-1, carrying the T-DNA construct, pNAP739. Agrobacterium-transformed cells undergo selection (e.g., for at least about 2 weeks, at least about 4 weeks, at least about 6 weeks, at least about 8 weeks, at least about 3 months, at least about 4 months, at least about 5 months or at least about 6 months) on medium containing both chlorsulfuron and sulfadiazine. The events from this selection are subjected to PCR analyses to reconfirm the presence of the integrated Donor DNA as well as the enrichment of the engineered plastid DNA in the cellular pool of plastid genomes.

The assessment of the percent replacement of wild-type plastid DNA with engineered plastid DNA is performed by a duplex PCR analysis as follows. One pair of primers (Donor-3 and Donor-4), specific to the Donor DNA, have the following sequences:

Donor-3:
(SEQ ID NO: 54)
GCTACTGGTGTTGGGCAACATCAA
Donor-4:
(SEQ ID NO: 55)
ATATGTATGCGCCCTATTCGCCTTA

A second pair of primers (CH2-WT-1 and CH2-WT-2), specific to the wild-type plastid DNA (SEQ ID NO: 93) targeted for replacement by the Donor DNA, have the following sequences:

CH2-WT-1:
(SEQ ID NO: 94)
ACACACATTGCTAACACTAGAAAACTTCC
CH2-WT-2:
(SEQ ID NO: 95)
CGGACCTGGAGTTTACCTATATACGGA

All primers have similar Tₘ temperatures of around 60-61° C. to allow for similar annealing efficiency in the duplex PCR reactions. Each duplex PCR reaction is set up with the following: 10 μmol of each of the four primers; OneTaq® DNA polymerase (New England Biolabs, Inc.); and total cell DNA from a callus sample (approximately 100 ng). The duplex PCR assay conditions are as follows: 94° C. for 30 sec; then 30 or 32 cycles of 94° C. for 20 sec, 54° C. for 30 sec, 68° C. for 30 sec; followed by a final incubation of 68° C. for 5 min. PCR products are then separated on a 1.5% agarose gel.

Gel images are analyzed using ImageJ, which is a Java-based image processing program developed at the National Institutes of Health and the Laboratory for Optical and Computational Instrumentation (LOCI, University of Wisconsin). Similar to that of mitochondrial transformation events, treatment with TALENs enhance % replacement of a wild-type plastid DNA region with a donor DNA.

### Example 6: Replacement of a Wild-Type Plastid DNA from the Inverted Repeat Region with Donor DNA

Wild-type callus cells were induced from the embryos of mature seed of rice, Oryza sativa L. ssp. Japonica cv. Nipponbare, and were co-transformed with the Donor DNA fragment of the plastid-expression construct pNAP662 and the plasmid DNA of the nuclear-expression construct pNAP199 by use of the biolistic method. The Donor DNA fragment (SEQ ID NO: 107) of plasmid pNAP662 contained an mALS (HR-LS) coding region operably linked to a rice plastid 16SrRNA promoter (SEQ ID NO: 70), a rice plastid psbA 5′-UTR (SEQ ID NO: 71), and a rice plastid psbA terminator (SEQ ID NO: 72). The Donor DNA had rice plastid DNA homologous regions at the 5′ and 3′ ends of 2 kb (SEQ ID NO: 108) and 2 kb (SEQ ID NO: 109), respectively. The homologous regions of the Donor DNA of plasmid pNAP662 targeted the region between the rps12 gene and the 16S rRNA gene in an inverted repeat region of the plastid DNA. In the earlier EXAMPLE 3, a Donor DNA of plasmid pNAP670 was targeted to a single-copy region of the plastid genome, e.g., upstream of the plastid rpoB gene. Plasmid pNAP199 had the following nuclear expression cassette: CaMV 35S promoter::HPT::CaMV 35S terminator (SEQ ID NO: 75). Using the mALS (HR-LS) and HPT genes as positive selectable markers for chlorsulfuron and hygromycin resistance, respectively, plastid transformants were selected as described for mitochondrial transformants in International Patent Application Publication No. WO2024187111A2 (Published Sep. 12, 2024). The events were further screened for the correct integration of the Donor DNA at the targeted plastid genome site, specifically, between the rps12 gene and the 16S rRNA gene. To confirm the correct integration of Donor DNA into the plastid genome, PCR analyses of the junction regions were performed. Each junction region was amplified with a primer specific to the Donor DNA region and another primer specific to plastid genomic sequence in the vicinity of the homologous region of Donor DNA.

Primer 132 (SEQ ID NO: 110) and primer 133 (SEQ ID NO: 111) were used to amplify a 2061 bp 5′ junction fragment corresponding to the 5′ region of Donor DNA insertion. Primer 127 (SEQ ID NO: 112) and primer 143 (SEQ ID NO: 113) were used to amplify a 2483 bp 3′ junction fragment corresponding to the 3′ region of Donor DNA insertion. For PCR analyses, total DNA from callus of each herbicide resistant event (5-20 mg) was isolated and resuspended in 30 μl TE. The DNA yield was about 420 ng/μl on average. 1 μl of DNA was used for each PCR reaction. The PCR reaction was prepared as follows: 1 μl of total DNA, 100 μM of each primer and 10 μl LongAmp Taq 2× Master Mix (New England Labs Inc.) in a 20 μl reaction mix. The PCR reaction for the 5′ junction amplification was performed as follows: 2 minutes at 94° C.; then 40 cycles of 15 seconds at 94° C., 30 sec at 59° C., and 2 minutes 10 seconds at 65° C.; followed by the final incubation for 10 minutes at 65° C. The PCR reaction for the 3′ junction amplification was performed as follows: 2 minutes at 94° C.; then 40 cycles of 15 seconds at 94° C., 30 seconds at 59° C., and 2 minutes 30 seconds at 65° C.; followed by the final incubation for 10 minutes at 65° C. The PCR samples were separated on 0.7% agarose gels.

### Example 7: Construction of TALENs Vector pNAP738 to Enhance Replacement of a Wild-Type Plastid DNA from the Inverted Repeat Region with Donor DNA

A pair of TALENs were designed to recognize plastid sequences that were separated by 16 bp, with the two TALEN recognition sites present on complementary DNA strands. One member of a TALEN pair, ChlA-F, targeted the following 18-mer sequence of the rice wild-type plastid DNA:

(SEQ ID NO: 114)
CAATTGTCAACCGGTCCT

The corresponding other member of the TALEN pair, ChlA-R, targeted the following 18-mer sequence:

(SEQ ID NO: 115)
ATGGCTCGAATCCATAGT

The coding sequences for the corresponding TALENs were synthesized in the form of mRNA by the vendor, Thermo Fisher Scientific, Inc. The TALEN sequences encoded in each mRNA were converted into the corresponding DNA by reverse transcriptase reactions and the DNA fragments were cloned into a Gateway vector.

Subsequently, each TALEN ORF was modified to have a chloroplast (plastid) targeting sequence (CTS) instead of a nuclear targeting sequence (NLS), and the sequences of constant regions were altered to avoid internal homologous recombination. Additionally, the FokI nuclease domain of each TALEN was replaced with the NAP12 nuclease domain. The resulting ORFs were each operably linked to a promoter and a terminator sequence for expression in the nucleus. The resulting two TALEN expression units, encoding CTS-ChlA-F (SEQ ID NO: 117) and CTS-ChlA-R (SEQ ID NO: 119), were cloned into a binary construct carrying a selectable marker gene for nuclear transformation encoding MTS(AT5G47030)-Sul1 (SEQ ID NO: 46), the Sul1 protein with the mitochondrial targeting sequence from AT5G47030, which confers resistance to the sulfonamide, sulfadiazine (SDZ). The binary vector also contained an expression unit for producing MTS(rps10)-folk (SEQ ID NO: 49), the folk protein with the mitochondrial targeting sequence from rice rps10.

The resulting T-DNA construct in pCAMBIA0380 vector, pNAP738, had the following expression cassettes in the region transferred to the nuclear genome:
OsTubA1 promoter::CTS (rbcS)-ChlA-F::HSP18.2 terminator (SEQ ID NO: 120);
ZmUbi1 promoter::CTS (rbcS)-ChlA-R::OCS terminator (SEQ ID NO: 121);
OsActin1 promoter::MTS(AT5G47030)-Sul1::NOS terminator (SEQ ID NO: 52);
CaMV 35S promoter::MTS(rps10)-folK::CaMV 35S terminator (SEQ ID NO: 53).

### Example 8: Use of TALENs to Enhance Replacement of a Wild-Type Plastid DNA from the Inverted Repeat Region with a Donor DNA

To eliminate wild-type plastid DNA in the events transformed with plastid Donor DNA, selected plastid transgenic events are re-transformed with the Agrobacterium strain, AGL-1, carrying the T-DNA construct, pNAP738. Agrobacterium-transformed cells undergo selection (e.g., for at least about 2 weeks, at least about 4 weeks, at least about 6 weeks, at least about 8 weeks, at least about 3 months, at least about 4 months, at least about 5 months or at least about 6 months) on medium containing both chlorsulfuron and sulfadiazine. The events from this selection are subjected to PCR analyses to reconfirm the presence of the integrated Donor DNA as well as the enrichment of the engineered plastid DNA in the cellular pool of plastid genomes.

The assessment of the percent replacement of wild-type plastid DNA with engineered plastid DNA is performed by a duplex PCR analysis as follows. One pair of primers (Donor-3 and Donor-4), specific to the Donor DNA, have the following sequences:

Donor-3:
(SEQ ID NO: 54)
GCTACTGGTGTTGGGCAACATCAA
Donor-4:
(SEQ ID NO: 55)
ATATGTATGCGCCCTATTCGCCTTA

A second pair of primers (CH1-WT-1 and CH1-WT-2), specific to the wild-type plastid DNA (SEQ ID NO: 122) targeted for replacement by the Donor DNA, have the following sequences:

CH1-WT-1:
(SEQ ID NO: 123)
CTCCGGGCTTTCTTTCCACAC
CH1-WT-2:
(SEQ ID NO: 124)
AATCGTACAGATTTTATGAAACCATGTGCT

All primers have similar Tₘ temperatures of around 60-61° C. to allow for similar annealing efficiency in the duplex PCR reactions. Each duplex PCR reaction is set up with the following: 10 μmol of each of the four primers; OneTaq® DNA polymerase (New England Biolabs, Inc.); and total cell DNA from a callus sample (approximately 100 ng). The duplex PCR assay conditions are as follows: 94° C. for 30 seconds; then 28 or 30 cycles of 94° C. for 20 seconds, 54° C. for 30 seconds, 68° C. for 30 seconds; followed by a final incubation of 68° C. for 5 minutes. PCR products are then separated on a 1.5% agarose gel. The 318 bp DNA band corresponds to Donor DNA and the 139 bp DNA band to the wild-type plastid DNA.

Gel images are analyzed using ImageJ, which is a Java-based image processing program developed at the National Institutes of Health and the Laboratory for Optical and Computational Instrumentation (LOCI, University of Wisconsin). Similar to that of mitochondrial transformation events, treatment with TALENs enhance % replacement of a wild-type plastid DNA region with a donor DNA.

### Example 9: Alternate Method of Calculating Donor DNA Replacement Percentages

A first method for calculating the Donor DNA replacement percentage used PCR reactions and assumed that two distinct PCR DNA fragments, one representing Donor DNA and the other wild-type mtDNA that was replaced by Donor DNA, were amplified with the same efficiency. For this purpose, PCR primers selected for the amplification of both DNA fragments had the same range of Tm values. However, PCR amplification can be influenced by internal sequences, as well. To assess the efficiency of PCR amplification of the two DNA sequences, we conducted a control experiment in which we used a plasmid DNA carrying the sequences of both DNA fragments. That is, this control PCR reaction had exactly the same number of molecules for each DNA template. The control plasmid was synthesized in the pUC-GW-Kan vector and named pNAP974. The insert of the control plasmid pNAP974 contained SEQ ID NO: 125, which has one copy of a wild-type mtDNA fragment (303-nt; underlined) replaced by Donor DNA, and one copy of a Donor DNA fragment (418-nt; in bold).

Using pNAP974 as a template, we performed PCR reactions as described in previous Examples. We discovered that the amplification of the two PCR fragments was not equal. The fragment of the wild-type mtDNA was amplified more efficiently than the Donor DNA fragment. To accommodate this biased PCR reaction, we developed a second method for determining the replacement percentage using the pNAP974 as a standard for this advanced analysis.

Four variables were used for calculating the replacement amount accurately. These variables were: (1) D: Donor DNA intensity of pNAP974 PCR reaction, (2) W: Wild-type DNA intensity of pNAP974 PCR reaction, (3) d: Donor DNA intensity of a sample, and (4) w: Wild-type DNA intensity of a sample.

Since the same number of molecules were present for Donor DNA and wild-type DNA in the control PCR reaction with pNAP974, D should be 50% of the sum of D and W. However, the actual reaction differed from the assumption. To make an adjustment, we calculated the fold change (FD) of the control to make D to 50%.

$$FD = 0.5 ⨯ ( D + W ) / D$$

The Donor DNA intensity of a sample (d) could be normalized by FD.

Normalized Donor DNA intensity of a sample (nd)=d×FD=d×0.5×(D+W)/D.

Similarly, the wild-type DNA intensity of a sample could be normalized by the intensity of the control DNA. The fold change (FW) of the control to make W to 50% is:

$$FW = 0.5 ⨯ ( D + W ) / W$$

The wild-type DNA intensity of a sample (w) could be normalized by FW.

Normalized wild-type DNA intensity of a sample (nw)=w×FW=w×0.5×(D+W)/W.

Further, the replaced amount (R) of wild-type DNA with donor DNA could be calculated by the following:

$$R = nd / ( nd + nw ) \\ R = [ d ⨯ 0.5 ⨯ ( D + W ) / D ] / [ d ⨯ 0.5 ⨯ ( D + W ) / D + w ⨯ 0.5 ⨯ ( D + W ) / W ] \\ R = [ d ⨯ ( D + W ) / D ] / [ d ⨯ ( D + W ) / D + w ⨯ ( D + W ) / W ] \\ R = ( d / D ) / ( d / D + w / W ) \\ R = d / ( d + w ⨯ D / W )$$

The non-replaced amount (NR) of the wild-type fragment was given by a similar calculation:

$$NR = nw / ( nd + nw ) \\ NR = [ w ⨯ 0.5 ⨯ ( D + W ) / W ] / [ d ⨯ 0.5 ⨯ ( D + W ) / D + w ⨯ 0.5 ⨯ ( D + W ) / W ] \\ NR = [ w ⨯ ( D + W ) / D ] / [ d ⨯ ( D + W ) / D + w ⨯ ( D + W ) / W ] \\ NR = w / W / ( d / D + w / W ) \\ NR = w / ( w + d ⨯ W / D )$$

The replaced (R) and non-replaced (NR) amounts from the above equations were calculated as a fractional rate. The sum of the R and NR was equal to one. The replacement percentage was calculated by R×100%. Likewise, the non-replacement percentage was calculated by NR×100%.

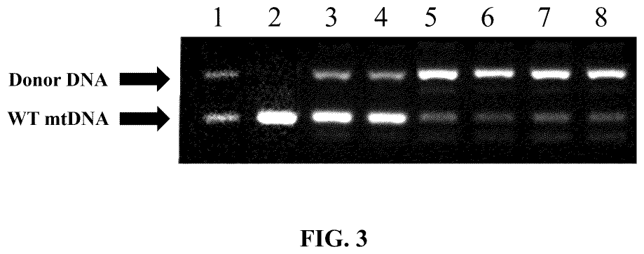

FIG. 3 shows PCR amplification of Donor DNA fragments and replaced wild-type fragments for three regenerated transgenic plants. PCR of the control plasmid pNAP974 is shown in lane 1 and PCR of wild-type rice DNA is shown in lane 2. Lanes 3-8 show PCR results for three regenerated plants, designated #1, #2, and #3. For each plant, young leaves (yl) and older leaves (ol) were assayed. The old and new methods were used to calculate the replacement amounts for the donor DNA. The results are shown in Table 4.

TABLE 4
Replacement Percentages as Calculated by 1st and 2nd Methods

| Lane Number | 3 | 4 | 5 | 6 | 7 | 8 |
|---|---|---|---|---|---|---|
| Tissue Sample | #1-yl | #1-ol | #2-yl | #2-ol | #3-yl | #3-ol |
| 1st Method | 21% | 22% | 66% | 71% | 68% | 69% |
| 2nd Method | 44% | 45% | 83% | 84% | 84% | 84% |

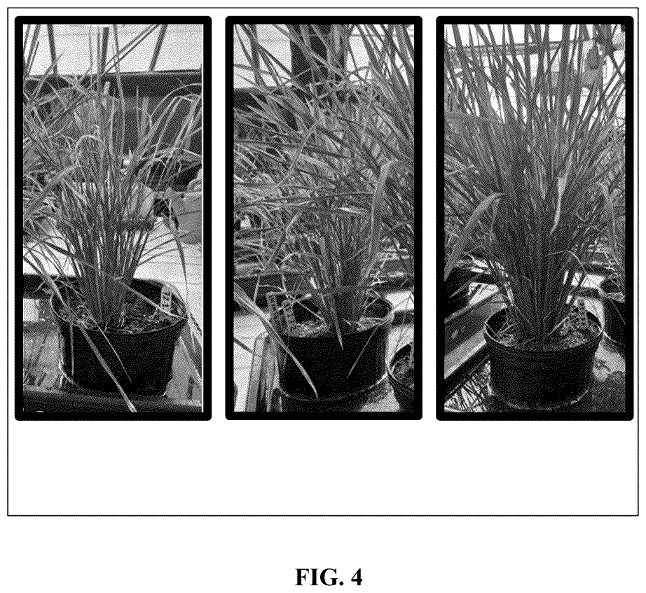

The regenerated plants in this experiment, shown in FIG. 4, were obtained from transgenic callus tissue that was secondarily transformed via the Agrobacterium method with TALEN plasmid pNAP651. This plasmid was described in Example 2 and contains the novel NAP12 nuclease domain. The Agrobacterium transformation was performed on callus tissue that initially had been transformed via the bombardment method with pNAP432 (described in Example 2) and pNAP159 (described in Example 1). Plasmid pNAP432 contains the orf79 coding region and the mALS (HR-LS) selectable marker gene. Plasmid pNAP159 contains the MTS-T7 polymerase expression unit and the HPT selectable marker gene.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### BRIEF DESCRIPTION OF THE SEQUENCES

A full list of the sequences disclosed in this application is provided in Table 3. Information regarding the sequences is provided below and throughout the description and examples of the specification:

SEQ ID NO: 1 (Artificial sequence) corresponds to the amino acid sequence of LAGLIDADG (SEQ ID NO: 1), a conserved sequence motif for one of the four Meganuclease families.

SEQ ID NO: 2 (Planomicrobium okeanokoites) corresponds to the amino acid sequence of the FokI endonuclease.

SEQ ID NO: 3 (Planomicrobium okeanokoites) corresponds to the amino acid sequence that comprises the functional nuclease domain of SEQ ID NO: 2.

SEQ ID NO: 4 (Lysobacter) corresponds to the amino acid sequence of the NAP12 endonuclease, which is the name used in this study for a 362-aa putative restriction endonuclease from Lysobacter (GenBank Accession No. WP_056305938.1).

SEQ ID NO: 5 (Lysobacter) corresponds to the amino acid sequence that comprises the functional nuclease domain of SEQ ID NO: 4.

SEQ ID NO: 6 (Artificial sequence) corresponds to the amino acid sequence (393-aa) of a MTS-Rep polypeptide. The amino-terminal 36-aa MTS was derived from the protein encoded by the Arabidopsis gene At5G47030.

SEQ ID NO: 7 (Beet curly top virus) corresponds to the nucleotide sequence (201-nt) of a geminivirus VOR element.

SEQ ID NO: 8 (Oryza sativa) corresponds to the amino acid sequence of the orf79 protein associated with CMS in rice.

SEQ ID NO: 9 (Triticum aestivum) corresponds to the amino acid sequence of the orf256 protein associated with CMS in wheat.

SEQ ID NO: 10 (Triticum aestivum) corresponds to the amino acid sequence of the orf279 protein associated with CMS in wheat.

SEQ ID NO: 11 (Artificial sequence) corresponds to the amino acid sequence of mALS (HR-LS), the herbicide-resistant ALS large subunit protein lacking a functional amino-terminal chloroplast transit sequence, for expression in mitochondria.

SEQ ID NO: 12 (Artificial sequence) corresponds to the nucleotide sequence encoding mALS (HR-LS) which was modified by removal of certain restriction sites and was optimized for expression in rice mitochondria by replacing rare codons for mitochondria with more frequently used codons.

SEQ ID NO: 13 (Artificial sequence) corresponds to the amino acid sequence of the MTS-ALS (SS) fusion protein encoded by SEQ ID NO: 12. The first 36 amino acids correspond to the MTS of the Arabidopsis At5g47030 gene.

SEQ ID NO: 14 (Artificial sequence) corresponds to the amino acid sequence (445 aa) of the mEPSPS-HR protein that is lacking a chloroplast transit peptide. Two amino acids residues that can confer resistance to glyphosate in the EPSPS-HR protein are isoleucine at position 103 and serine at position 107 (shown in bold font in TABLE 3).

SEQ ID NO: 15 (Artificial sequence) corresponds to the optimized nucleotide sequence (1338 nt) of the mEPSPS-HR coding region. In TABLE 3, lower case letters were the nucleotides modified from the corresponding rice EPSPS gene.

SEQ ID NO: 16 (Artificial sequence) corresponds to the amino acid sequence (370 aa) of the herbicide-resistant GS1-HR protein. One amino acid residue at position 61 (underlined and in bold font in TABLE 3) can confer resistance to glufosinate.

SEQ ID NO: 17 (Artificial sequence) corresponds to the nucleotide sequence (1110 nt) of the optimized GS1-HR coding region minus the translation initiation codon. In TABLE 3, lower case letters indicate the nucleotides modified from the corresponding wild-type rice GS1 gene.

SEQ ID NO: 18 (Oryza sativa) corresponds to the nucleotide sequence encoding an RNA editing site of the rice mitochondrial nad4L transcript.

SEQ ID NO: 19 (Oryza sativa) corresponds to the nucleotide sequence encoding an RNA editing site of the rice mitochondrial cox2 transcript.

SEQ ID NO: 20 (Artificial sequence) corresponds to the nucleotide sequence (75 nt) that encodes the wheat mitochondrial cox2 RNA editing site at the cox2 translation start site. In TABLE 3, the sequence derived from the wheat cox2 RNA editing site is underlined and the C nucleotide at position 41 that is edited in U in the corresponding RNA is in bold font.

SEQ ID NO: 21 (Artificial sequence) corresponds to the amino acid sequence of a hydrophobic quenching peptide that tetramerizes GFP and prevents maturation of the chromophore.

SEQ ID NO: 22 (Artificial sequence) corresponds to the amino acid sequence of a caspase recognition sequence.

SEQ ID NO: 23 (Triticum aestivum) corresponds to the nucleotide sequence for a candidate RNA editing sequence present in the wheat mitochondrial cox2 gene at position 449 of the gene.

SEQ ID NO: 24 (Triticum aestivum) corresponds to the nucleotide sequence for a candidate RNA editing sequence present in the wheat mitochondrial cox2 gene at position 587 of the gene.

SEQ ID NO: 25 (Triticum aestivum) corresponds to the nucleotide sequence for a candidate RNA editing sequence present in the wheat mitochondrial cox2 gene at position 620 of the gene.

SEQ ID NO: 26 (Artificial sequence) corresponds to the nucleotide sequence of the pNAP433 Donor DNA fragment having the rice mitochondrial cox2 RNA editing site. In TABLE 3, the homologous regions are underlined, the mALS (HR-LS) ORF is highlighted in bold, and the RNA editing site to create AUG in mitochondria is shown in lower case.

SEQ ID NO: 27 (Artificial sequence) corresponds to the nucleotide sequence of a hybrid T7+ATP1 terminator.

SEQ ID NO: 28 (Artificial sequence) corresponds to the nucleotide sequence of a truncated version of the hybrid T7+ATP1 terminator presented as SEQ ID NO: 29.

SEQ ID NO: 29 (Oryza sativa) corresponds to the nucleotide sequence encoding an orf79 protein.

SEQ ID NO: 30 (Artificial sequence) corresponds to the nucleotide sequence encoding a gRNA cassette for use with a MAD7 nuclease.

SEQ ID NO: 31 (Artificial sequence) corresponds to the amino acid sequence of MAD7.

SEQ ID NO: 32 (Artificial sequence) corresponds to the nucleotide sequence of the 5′ homologous region in the Donor DNA of plasmids pNAP432 and pNAP433. Certain nucleotides were changed in the 5′ homologous region to prevent future recognition by gRNA2 and the MAD7 enzyme.

SEQ ID NO: 33 (Artificial sequence) corresponds to the nucleotide sequence of the 3′ homologous region of the Donor DNA of plasmids pNAP432 and pNAP433. Certain nucleotides were changed in the 3′ homologous region to prevent future recognition by gRNA4 and the MAD7 enzyme.

SEQ ID NO: 34 (Artificial sequence) corresponds to the nucleotide sequence encoding the MTS-T7 RNA polymerase. The first 108 nucleotides encode the MTS derived from At5g47030.

SEQ ID NO: 35 (Artificial sequence) corresponds to the amino acid sequence of the MTS-T7 RNA polymerase. The first 36 amino acids are the MTS derived from At5g47030.

SEQ ID NO: 36 (Arabidopsis thaliana) corresponds to the amino acid sequence of the 36-aa MTS derived from At5g47030.

SEQ ID NO: 37 (Oryza sativa) corresponds to the nucleic acid sequence of the 505-bp region of rice mitochondrial DNA to be replaced by the Donor DNA.

SEQ ID NO: 38 (Oryza sativa) corresponds to the nucleic acid sequence of the 18-nt region of the rice mitochondrial DNA targeted by TAL-C.

SEQ ID NO: 39 (Oryza sativa) corresponds to the nucleic acid sequence of the 18-nt region of the rice mitochondrial DNA targeted by TAL-D.

SEQ ID NO: 40 (Artificial sequence) corresponds to the nucleic acid sequence of the modified ORF for MTS-TAL-C, which has a mitochondrial targeting sequence (MTS) instead of a nuclear targeting sequence (NLS), and in which sequences of the constant regions were altered to avoid internal homologous recombination. The 168-nt MTS coding region derived from the rice rps10 gene is underlined in TABLE 3.

SEQ ID NO: 41 (Artificial sequence) corresponds to the amino acid sequence of the MTS-TAL-C protein encoded by SEQ ID NO: 40. The 56-aa MTS derived from the rice rps10 protein is underlined in TABLE 3.

SEQ ID NO: 42 (Oryza sativa) corresponds to the amino acid sequence of the 56-aa MTS derived from the rice rps10 protein (GenBank No. ABA98720.1).

SEQ ID NO: 43 (Artificial sequence) corresponds to the nucleic acid sequence of the modified ORF for MTS-TAL-D, which has a mitochondrial targeting sequence (MTS) instead of a nuclear targeting sequence (NLS), and in which sequences of the constant regions were altered to avoid internal homologous recombination. The 108-nt MTS coding region derived from the Arabidopsis thaliana AT5G47030 gene is underlined in TABLE 3.

SEQ ID NO: 44 (Artificial sequence) corresponds to the amino acid sequence of the MTS-TAL-D protein encoded by SEQ ID NO: 43. The 36-aa MTS derived from the Arabidopsis thaliana AT5G47030 protein is underlined in TABLE 3.

SEQ ID NO: 45 (Artificial sequence) corresponds to the nucleic acid sequence coding for the MTS-Sul1 protein. The 108-nt MTS coding region derived from the Arabidopsis thaliana AT5G47030 gene is underlined in TABLE 3.

SEQ ID NO: 46 (Artificial sequence) corresponds to the amino acid sequence of the MTS-Sul1 protein encoded by SEQ ID NO: 45. The 36-aa MTS derived from the Arabidopsis thaliana AT5G47030 protein is underlined in TABLE 3.

SEQ ID NO: 47 (Escherichia coli) corresponds to the amino acid sequence of the Sul1 protein.

SEQ ID NO: 48 (Artificial sequence) corresponds to the nucleic acid sequence encoding the MTS-folk protein. The 168-nt MTS coding region derived from the rice rps10 gene is underlined in TABLE 3.

SEQ ID NO: 49 (Artificial sequence) corresponds to the amino acid sequence of the MTS-folK protein encoded by SEQ ID NO: 48. The 56-aa MTS derived from the rice rps10 protein is underlined in TABLE 3.

SEQ ID NO: 50 (Artificial sequence) corresponds to the nucleic acid sequence of the expression unit comprising the MTS(rps10)-TAL-C coding region operably linked to the OsTubA1 promoter & intron and to the HSP18.2 terminator. The MTS(rps10)-TAL-C coding region is in bold font in TABLE 3.

SEQ ID NO: 51 (Artificial sequence) corresponds to the nucleic acid sequence of the expression unit comprising the MTS(AT5G47030)-TAL-D coding region operably linked to the ZmUbi1 promoter & intron and to the OCS terminator. The MTS(AT5G47030)-TAL-D coding region is in bold font in TABLE 3.

SEQ ID NO: 52 (Artificial sequence) corresponds to the nucleic acid sequence of the expression unit comprising the MTS(AT5G47030)-Sul1 coding region operably linked to the OsActin1 promoter & intron and to the NOS terminator. The MTS(AT5G47030)-Sul1coding region is in bold font in TABLE 3.

SEQ ID NO: 53 (Artificial sequence) corresponds to the nucleic acid sequence of the expression unit comprising the MTS(rps10)-folK coding region operably linked to the CaMV 35S promoter and to the CaMV 35S terminator. The MTS(rps10)-folk coding region is in bold font in TABLE 3.

SEQ ID NO: 54 (Artificial sequence) corresponds to the nucleic acid sequence for the Donor-3 PCR primer. It is used to amplify a region of the mALS (HR-LS) coding sequence in the Donor DNA.

SEQ ID NO: 55 (Artificial sequence) corresponds to the nucleic acid sequence for the Donor-4 PCR primer. It is used to amplify a region of the mALS (HR-LS) coding sequence in the Donor DNA.

SEQ ID NO: 56 (Oryza sativa) corresponds to the nucleic acid sequence for the WT-2 PCR primer. It is used to amplify a portion of 505-bp region of rice mtDNA to be replaced by the Donor DNA.

SEQ ID NO: 57 (Oryza sativa) corresponds to the nucleic acid sequence for the WT-3 PCR primer. It is used to amplify a portion of 505-bp region of rice mtDNA to be replaced by the Donor DNA.

SEQ ID NO: 58 (Artificial sequence) corresponds to the nucleotide sequence of the pNAP432 Donor DNA fragment having the rice mitochondrial nad4L RNA editing site. In TABLE 3, the homologous regions are underlined, the mALS (HR-LS) ORF is highlighted in bold, and the RNA editing site to create AUG in mitochondria is shown in lower case.

SEQ ID NO: 59 (Artificial sequence) corresponds to the nucleotide sequence encoding the MTS-ALS (SS) fusion protein presented as SEQ ID NO: 13.

SEQ ID NO: 60 (Lysobacter) corresponds to the amino acid sequence of the 198-aa NAP12 nuclease region used in this study. The 198-aa NAP12 nuclease region includes the 161-aa amino-terminal nuclease domain (SEQ ID NO: 5) and the 37-aa linker region (SEQ ID NO: 61) between the nuclease domain and the carboxy-terminal DNA-binding domain.

SEQ ID NO: 61 (Lysobacter) corresponds to the amino acid sequence of the NAP12 37-aa linker region between the amino-terminal nuclease domain and the carboxy-terminal DNA-binding domain.

SEQ ID NO: 62 (Oryza sativa) corresponds to the nucleic acid sequence of the 18-nt region of the rice mitochondrial DNA targeted by TAL-C1.

SEQ ID NO: 63 (Artificial sequence) corresponds to the nucleic acid sequence of the modified ORF for MTS-TAL-C1-NAP12, which has a mitochondrial targeting sequence (MTS) instead of a nuclear targeting sequence (NLS), where sequences of the constant regions were altered to avoid internal homologous recombination, and where the FokI nuclease domain has been replaced with the NAP12 nuclease domain. The 168-nt MTS coding region derived from the rice rps10 gene is underlined in TABLE 3.

SEQ ID NO: 64 (Artificial sequence) corresponds to the amino acid sequence of the MTS-TAL-C1-NAP12 protein encoded by SEQ ID NO: 63. The 56-aa MTS derived from the rice rps10 protein is underlined in TABLE 3.

SEQ ID NO: 65 (Artificial sequence) corresponds to the nucleic acid sequence of the modified ORF for MTS-TAL-D-NAP12, which has a mitochondrial targeting sequence (MTS) instead of a nuclear targeting sequence (NLS), where sequences of the constant regions were altered to avoid internal homologous recombination, and where the FokI nuclease domain has been replaced with the NAP12 nuclease domain. The 108-nt MTS coding region derived from the Arabidopsis thaliana AT5G47030 gene is underlined in TABLE 3.

SEQ ID NO: 66 (Artificial sequence) corresponds to the amino acid sequence of the MTS-TAL-D-NAP12 protein encoded by SEQ ID NO: 65. The 36-aa MTS derived from the Arabidopsis thaliana AT5G47030 protein is underlined in TABLE 3.

SEQ ID NO: 67 (Artificial sequence) corresponds to the nucleic acid sequence of the expression unit comprising the MTS(rps10)-TAL-C1-NAP12 coding region operably linked to the OsTubA1 promoter & intron and to the HSP18.2 terminator. The MTS(rps10)-TAL-C1-NAP12 coding region is in bold font in TABLE 3.

SEQ ID NO: 68 (Artificial sequence) corresponds to the nucleic acid sequence of the expression unit comprising the MTS(AT5G47030)-TAL-D-NAP12 coding region operably linked to the ZmUbi1 promoter & intron and to the OCS terminator. The MTS(AT5G47030)-TAL-D-NAP12 coding region is in bold font in TABLE 3.

SEQ ID NO: 69 (Artificial sequence) corresponds to the nucleic acid sequence of the Donor DNA fragment of plasmid pNAP670. In TABLE 3, the mALS (HR-LS) coding region is shown in bold font, the rice plastid 16S rRNA promoter is underlined, and the psbA 5′-UTR and psbA terminator are shown in italic font.

SEQ ID NO: 70 (Oryza sativa) corresponds to the nucleic acid sequence of the rice plastid 16S rRNA promoter in plasmid pNAP670.

SEQ ID NO: 71 (Oryza sativa) corresponds to the nucleic acid sequence of the rice plastid psbA 5′-untranslated region (5′-UTR) in plasmid pNAP670.

SEQ ID NO: 72 (Oryza sativa) corresponds to the nucleic acid sequence of the rice plastid psbA terminator region in plasmid pNAP670.

SEQ ID NO: 73 (Oryza sativa) corresponds to the nucleic acid sequence of the 2-kb 5′ homologous region in the Donor DNA of plasmid pNAP670.

SEQ ID NO: 74 (Oryza sativa) corresponds to the nucleic acid sequence of the 2-kb 3′ homologous region in the Donor DNA of plasmid pNAP670.

SEQ ID NO: 75 (Artificial sequence) corresponds to the nucleic acid sequence of the CaMV 35S promoter::HPT::CaMV terminator expression unit in pNAP199.

SEQ ID NO: 76 (Oryza sativa) corresponds to the nucleic acid sequence of PCR primer rpoB_rev_3.

SEQ ID NO: 77 (Artificial sequence) corresponds to the nucleic acid sequence of PCR primer us_ALS_rev_1.

SEQ ID NO: 78 (Oryza sativa) corresponds to the nucleic acid sequence of PCR primer pED10_fwd_2.

SEQ ID NO: 79 (Artificial sequence) corresponds to the nucleic acid sequence of PCR primer ALS_D626_for.

SEQ ID NO: 80 (Artificial sequence) corresponds to the nucleic acid sequence of a 160-bp region of the 2237-bp 5′ junction fragment that has 80-bp of unique plastid DNA sequence and 80-bp of 5′-HR DNA sequence that is present in both the Donor DNA and the wild-type plastid DNA.

SEQ ID NO: 81 (Artificial sequence) corresponds to the nucleic acid sequence of a 129-bp region of the 2237-bp 5′ junction fragment that has 80-bp of 5′-HR DNA that is present in both the Donor DNA and the wild-type plastid DNA and 49-bp of unique Donor DNA sequence.

SEQ ID NO: 82 (Artificial sequence) corresponds to the nucleic acid sequence of a 133-bp region of the 2207-bp 3′ junction fragment that has 80-bp of unique Donor DNA sequence and 53-bp of 3′-HR DNA sequence that is present in both the Donor DNA and wild-type plastid DNA.

SEQ ID NO: 83 (Artificial sequence) corresponds to the nucleic acid sequence of a 110-bp region of the 2207-bp 3′ junction fragment that has 81-bp of 3′-HR DNA sequence that is present in both Donor DNA and wild-type plastid DNA and 29-bp of unique plastid DNA sequence.

SEQ ID NO:84 (Oryza sativa) corresponds to the nucleic acid sequence of the 18-nt region of rice plastid DNA targeted by Chl-TAL3.

SEQ ID NO: 85 (Oryza sativa) corresponds to the nucleic acid sequence of the 18-nt region of rice plastid DNA targeted by Chl-TAL4.

SEQ ID NO: 86 (Artificial sequence) corresponds to the nucleic acid sequence of the modified ORF for CTS-Chl-TAL3, which has a chloroplast targeting sequence (CTS) instead of a nuclear targeting sequence (NLS), and in which sequences of the constant regions were altered to avoid internal homologous recombination. The 135-nt CTS coding region derived from the rice rbsS gene is underlined in TABLE 3.

SEQ ID NO: 87 (Artificial sequence) corresponds to the amino acid sequence of the CTS-Chl-TAL3 protein encoded by SEQ ID NO: 86. The 45-aa CTS derived from the rice rbcs protein is underlined in TABLE 3.

SEQ ID NO: 88 (Artificial sequence) corresponds to the amino acid sequence of the 45-aa CTS derived from the rice rbcS protein.

SEQ ID NO: 89 (Artificial sequence) corresponds to the nucleic acid sequence of the modified ORF for CTS-Chl-TAL4, which has a chloroplast targeting sequence (CTS) instead of a nuclear targeting sequence (NLS), and in which sequences of the constant regions were altered to avoid internal homologous recombination. The 135-nt CTS coding region derived from the rbcs gene is underlined in TABLE 3.

SEQ ID NO: 90 (Artificial sequence) corresponds to the amino acid sequence of the CTS-Chl-TAL4 protein encoded by SEQ ID NO: 89. The 45-aa MTS derived from the rbcs protein is underlined in TABLE 3.

SEQ ID NO: 91 (Artificial sequence) corresponds to the nucleic acid sequence of the 5934-nt transcription unit with the following elements: OsTubA1 promoter::CTS (rbcS)-Chl-TAL3::HSP18.2 terminator.

SEQ ID NO: 92 (Artificial sequence) corresponds to the nucleic acid sequence of the 5807-nt transcription unit with the following elements: ZmUbi1 promoter::CTS (rbcS)-Chl-TAL4::OCS terminator.

SEQ ID NO: 93 (Oryza sativa) corresponds to the nucleic acid sequence of the 368-bp region of wild-type rice plastid DNA targeted for replacement by the Donor DNA.

SEQ ID NO: 94 (Oryza sativa) corresponds to the nucleic acid sequence for the CH2-WT-1 PCR primer. It is used to amplify a portion of 368-bp region of rice plastid DNA to be replaced by the Donor DNA.

SEQ ID NO: 95 (Oryza sativa) corresponds to the nucleic acid sequence for the CH2-WT-2 PCR primer. It is used to amplify a portion of 368-bp region of rice plastid DNA to be replaced by the Donor DNA.

SEQ ID NO: 96 (Artificial sequence) corresponds to the amino acid sequence of the herbicide-resistant ALS large subunit protein encoded by the donor DNA of pNAP432 having the rice mitochondrial nad4L RNA editing site.

SEQ ID NO: 97 (Artificial sequence) corresponds to the amino acid sequence of the herbicide-resistant ALS large subunit protein encoded by the donor DNA of pNAP433 having the rice mitochondrial cox2 RNA editing site.

SEQ ID NO: 98 (Artificial sequence) corresponds to the amino acid sequence of the herbicide-resistant ALS large subunit protein encoded by the donor DNA of pNAP610.

SEQ ID NO: 99 (Oryza sativa) corresponds to the nucleic acid sequence of the 18-nt region of the rice mitochondrial DNA targeted by TAL-A1.

SEQ ID NO: 100 (Oryza sativa) corresponds to the nucleic acid sequence of the 18-nt region of the rice mitochondrial DNA targeted by TAL-B1.

SEQ ID NO: 101 (Artificial sequence) corresponds to the nucleic acid sequence of the modified ORF for MTS-TAL-A1-NAP12, which has a mitochondrial targeting sequence (MTS) instead of a nuclear targeting sequence (NLS), where sequences of the constant regions were altered to avoid internal homologous recombination, and where the FokI nuclease domain has been replaced with the NAP12 nuclease domain. The 168-nt MTS coding region derived from the rice rps10 gene is underlined in TABLE 3.

SEQ ID NO: 102 (Artificial sequence) corresponds to the amino acid sequence of the MTS-TAL-A1-NAP12 protein encoded by SEQ ID NO: 63. The 56-aa MTS derived from the rice rps10 protein is underlined in TABLE 3.

SEQ ID NO: 103 (Artificial sequence) corresponds to the nucleic acid sequence of the expression unit comprising the MTS(rps10)-TAL-A1-NAP12 coding region operably linked to the OsTubA1 promoter & intron and to the HSP18.2 terminator. The MTS(rps10)-TAL-A1-NAP12 coding region is in bold font in TABLE 3.

SEQ ID NO: 104 (Artificial sequence) corresponds to the nucleic acid sequence of the modified ORF for MTS-TAL-B1-NAP12, which has a mitochondrial targeting sequence (MTS) instead of a nuclear targeting sequence (NLS), where sequences of the constant regions were altered to avoid internal homologous recombination, and where the FokI nuclease domain has been replaced with the NAP12 nuclease domain. The 108-nt MTS coding region derived from the Arabidopsis thaliana AT5G47030 gene is underlined in TABLE 3.

SEQ ID NO: 105 (Artificial sequence) corresponds to the amino acid sequence of the MTS-TAL-B1-NAP12 protein encoded by SEQ ID NO: 65. The 36-aa MTS derived from the Arabidopsis thaliana AT5G47030 protein is underlined in TABLE 3.

SEQ ID NO: 106 (Artificial sequence) corresponds to the nucleic acid sequence of the expression unit comprising the MTS(AT5G47030)-TAL-B1-NAP12 coding region operably linked to the ZmUbi1 promoter & intron and to the OCS terminator. The MTS(AT5G47030)-TAL-B1-NAP12 coding region is in bold font in TABLE 3.

SEQ ID NO: 107 (Artificial sequence) corresponds to the nucleotide sequence of the pNAP662 Donor DNA fragment. In TABLE 3, the 5′ and 3′ homologous regions are underlined and the mALS (HR-LS) ORF is highlighted in bold.

SEQ ID NO: 108 (Artificial sequence) corresponds to the nucleotide sequence of the 5′ homologous region in the Donor DNA of plasmids pNAP662.

SEQ ID NO: 109 (Artificial sequence) corresponds to the nucleotide sequence of the 3′ homologous region in the Donor DNA of plasmids pNAP662.

SEQ ID NO: 110 (Artificial sequence) corresponds to the nucleotide sequence of PCR primer 132 used to amplify a 2061-bp 5′ junction fragment corresponding to the 5′ region of Donor DNA insertion from plasmid pNAP662.

SEQ ID NO: 111 (Artificial sequence) corresponds to the nucleotide sequence of PCR primer 133 used to amplify a 2061-bp 5′ junction fragment corresponding to the 5′ region of Donor DNA insertion from plasmid pNAP662.

SEQ ID NO: 112 (Artificial sequence) corresponds to the nucleotide sequence of PCR primer 127 used to amplify a 2483-bp 3′ junction fragment corresponding to the 3′ region of Donor DNA insertion from plasmid pNAP662.

SEQ ID NO: 113 (Artificial sequence) corresponds to the nucleotide sequence of PCR primer 143 used to amplify a 2483-bp 3′ junction fragment corresponding to the 3′ region of Donor DNA insertion from plasmid pNAP662.

SEQ ID NO: 114 (Oryza sativa) corresponds to the nucleic acid sequence of the 18-nt region of the rice plastid DNA targeted by ChlA-F.

SEQ ID NO: 115 (Oryza sativa) corresponds to the nucleic acid sequence of the 18-nt region of the rice plastid DNA targeted by ChlA-R.

SEQ ID NO: 116 (Artificial sequence) corresponds to the nucleic acid sequence of the modified ORF for CTS-ChlA-F, which has a chloroplast targeting sequence (CTS) instead of a nuclear targeting sequence (NLS), and in which sequences of the constant regions were altered to avoid internal homologous recombination. The 135-nt CTS coding region for a rice rbcS gene is underlined in TABLE 3. The 597-nt NAP12 nuclease domain coding region is in bold font in TABLE 3.

SEQ ID NO: 117 (Artificial sequence) corresponds to the amino acid sequence of the CTS-ChlA-F protein encoded by SEQ ID NO: 116. In TABLE 3 the 45-aa CTS derived from the rice rbcS protein is underlined and the 198-aa NAP12 nuclease domain is in bold font.

SEQ ID NO: 118 (Artificial sequence) corresponds to the nucleic acid sequence of the modified ORF for CTS-ChlA-R, which has a chloroplast targeting sequence (CTS) instead of a nuclear targeting sequence (NLS), and in which sequences of the constant regions were altered to avoid internal homologous recombination. The 135-nt CTS coding region for a rice rbcS gene is underlined in TABLE 3. The 597-nt NAP12 nuclease domain coding region is in bold font in TABLE 3.

SEQ ID NO: 119 (Artificial sequence) corresponds to the amino acid sequence of the CTS-ChlA-R protein encoded by SEQ ID NO: 118. In TABLE 3 the 45-aa CTS derived from the rice rbcS protein is underlined and the 198-aa NAP12 nuclease domain is in bold font.

SEQ ID NO: 120 (Artificial sequence) corresponds to the nucleic acid sequence of the expression unit comprising the CTS-ChlA-F coding region operably linked to the OsTubA1 promoter & intron and to the HSP18.2 terminator. The CTS-ChlA-F coding region is in bold font in TABLE 3. The sequences encoding the N-terminal rbcS plastid targeting sequence and the C-terminal NAP12 domain are underlined.

SEQ ID NO: 121 (Artificial sequence) corresponds to the nucleic acid sequence of the expression unit comprising the CTS-ChlA-R coding region operably linked to the ZmUbi1 promoter & intron and to the OCS terminator. The CTS-ChlA-R coding region is in bold font in TABLE 3. The sequences encoding the N-terminal rbcS plastid targeting sequence and the C-terminal NAP12 domain are underlined.

SEQ ID NO: 122 (Oryza sativa) corresponds to the nucleic acid sequence of the region of wild-type rice plastid DNA targeted for replacement by the Donor DNA of plasmid pNAP662.

SEQ ID NO: 123 (Oryza sativa) corresponds to the nucleic acid sequence for the CH1-WT-1 PCR primer. It is used to amplify a portion of the rice plastid DNA to be replaced by the Donor DNA of plasmid pNAP662.

SEQ ID NO: 124 (Oryza sativa) corresponds to the nucleic acid sequence for the CH1-WT-2 PCR primer. It is used to amplify a portion of the rice plastid DNA to be replaced by the Donor DNA of plasmid pNAP662.

SEQ ID NO: 125 (Artificial sequence) corresponds to the nucleotide sequence of the insert of the control plasmid pNAP974, which has one copy of a wild-type mtDNA fragment (303-nt; underlined) replaced by Donor DNA, and one copy of a Donor DNA fragment (418-nt; in bold).

SEQ ID NO: 126 (Oryza rufipogon) corresponds to the amino acid sequence of WA352a from Oryza rufipogon W-105 isolate (GenBank Accession No. AOT98974.1).

SEQ ID NO: 127 (Oryza rufipogon) corresponds to the amino acid sequence of WA352b from Oryza rufipogon 8w-12 isolate (GenBank Accession No. AOT98982.1).

SEQ ID NO: 128 (Oryza sativa) corresponds to the amino acid sequence of WA352c from Oryza sativa Indica Group ZS97A isolate (GenBank Accession No. AOT98984.1). The amino acid sequence of WA352c from Oryza sativa is identical to the amino acid sequence of WA352b from Oryza rufipogon 8w-12 isolate and has four amino acid differences with the amino acid sequence of WA352a from Oryza rufipogon W-105 isolate.

SEQ ID NO: 129 (Chlamydomonas reinhardtii) corresponds to the amino acid sequence of an MTS from a Chlamydomonas reinhardtii mitochondrial ubiquinol-cytochrome c reductase (GenBank Accession No. CAC86460.2). Cleavage of the precursor sequence occurs after amino acid number 55.

TABLE 3
Sequence Details

| SEQ | |
|---|---|
| ID | SEQUENCE |
| 1 | LAGLIDADG |
| 2 | MFLSMVSKIRTFGWVQNPGKFENLKRVVQVFDRNSKVHNEVKNIKIPTLVKESK |
| | IQKELVAIMNQHDLIYTYKELVGTGTSIRSEAPCDAIIQATIADQGNKKGYIDNWS |
| | SDGFLRWAHALGFIEYINKSDSFVITDVGLAYSKSADGSAIEKEILIEAISSYPPAIR |
| | ILTLLEDGQHLTKFDLGKNLGFSGESGFTSLPEGILLDTLANAMPKDKGEIRNNW |
| | EGSSDKYARMIGGWLDKLGLVKQGKKEFIIPTLGKPDNKEFISHAFKITGEGLKV |
| | LRRAKGSTKFTRVPKRVYWEMLATNLTDKEYVRTRRALILEILIKAGSLKIEQIQ |
| | DNLKKLGFDEVIETIENDIKGLINTGIFIEIKGRFYQLKDHILQFVIPNRGVTKQLV |
| | KSELEEKKSELRHKLKYVPHEYIELIEIARNSTQDRILEMKVMEFFMKVYGYRGK |
| | HLGGSRKPDGAIYTVGSPIDYGVIVDTKAYSGGYNLPIGQADEMQRYVEENQTR |
| | NKHINPNEWWKVYPSSVTEFKFLFVSGHFKGNYKAQLTRLNHITNCNGAVLSVE |
| | ELLIGGEMIKAGTLTLEEVRRKFNNGEINF |
| 3 | QLVKSELEEKKSELRHKLKYVPHEYIELIEIARNSTQDRILEMKVMEFFMKVYGY |
| | RGKHLGGSRKPDGAIYTVGSPIDYGVIVDTKAYSGGYNLPIGQADEMQRYVEEN |
| | QTRNKHINPNEWWKVYPSSVTEFKFLFVSGHFKGNYKAQLTRLNHITNCNGAV |
| | LSVEELLIGGEMIKAGTLTLEEVRRKFNNGEINF |
| 4 | MNELTLHPNVYATGASKGLIHVLERVWVREHTPGDGTIFVISGFGNYNGGVRFF |
| | ESFREHKRRGGELVAVFAGSTSARLTSKQVVHEMLDCGADVHIVNRKRLLHAK |
| | CYGTSKMSGDSLIVTSGNFTGPGMSQNVEMSVLLDADTTAEMGFSWGTLLENL |
| | LDQRWNYHRPTLGDPANPAWSLLYDEQAADIRLDETEEVTMLIRLSHADTARIN |
| | ATPGTSAGLGSQYFWLSKDCYDFFPPLTIRNQRGLRATYSCLISMLYVDLNVTD |
| | DNCRVTFEAENNLDFRLGTGPLRYTRLAAAGDLAAVTRLGENNYELRIYRAGA |
| | AEYQNLMPYAVNLIGHQGKQYGYISNAEFSAITGTRFRLRR |
| 5 | MNELTLHPNVYATGASKGLIHVLERVWVREHTPGDGTIFVISGFGNYNGGVRFF |
| | ESFREHKRRGGELVAVFAGSTSARLTSKQVVHEMLDCGADVHIVNRKRLLHAK |
| | CYGTSKMSGDSLIVTSGNFTGPGMSQNVEMSVLLDADTTAEMGFSWGTLLENL |
| | L |
| 6 | MFKQASRLLSRSVAAASSKSVTTRAFSTELPSTLDSPPTKRFRIQAKNIFLTYPQC |
| | SLSKEEALEQIQRIQLSSNKKYIKIARELHEDGQPHLHVLLQLEGKVQITNIRLFDL |
| | VSPTRSAHFHPNIQRAKSSSDVKSYVDKDGDTIEWGEFQIDGRSARGGQQTAND |
| | SYAKALNATSLDQALQILKEEQPKDYFLQHHNLLNNAQKIFQRPPDPWTPLFPLS |
| | SFTNVPEEMQEWADAYFGVDAAARPLRYNSIIVEGDSRTGKTMWARSLGAHNY |
| | ITGHLDFSPRTYYDEVEYNVIDDVDPTYLKMKHWKHLIGAQKEWQTNLKYGKP |
| | RVIKGGIPCIILCNPGPESSYQQFLEKPENEALKSWTLHNSTFCKLQGPLFNNQAA |
| | ASSQGDSTL |
| 7 | TGTACTCCGATGACGTGGCTTAGCATATTAACATATCTATTGGAGTATTGGAG |
| | TATTATATATATTAGTACAACTTTCATAAGGGCCATCCGTTATAATATTACCG |
| | GATGGCCCGAAAAAAATGGGCACCCAATCAAAACGTGACACGTGGAAGGGG |
| | ACTGTTGAATGATGTGACGTTTTTGAGCGGGAAACTTCCTGAAG |
| 8 | MANLVRWLFSTTRGTNGLPYFIFGVVVGGALLFALLKYQAPLYDPALMEKIIDH |
| | NIKAGHPIEVDYSWWGTSIRVVFPK |
| 9 | MTNMVRWLFSTSRFTAFYLHFCIKFPYIYDFLLFSICLFFFLVRFISIILLPICNLFGA |
| | SFIITLPPEIQDPQALAHLAGLNFYLSLYEQDPGWVTFIQNELNHNTPLEDIPGRLK |
| | LFLMEEKLSSMRQDVIQEFVALYQRIGPYLPIEPYLVDEALRSYLDHIHATDSFTV |
| | LQASYQDLRENEGGSVFFRDAVSHNRDLLEAESSARRCLEVEQRIRWEEIPKSKA |
| | SLERAEHEHALDLFKSEDLRRELEKKRAG |
| 10 | MPQLDKLTYFSQFFWLCLLLFTFYILLFNNNNGILGISRILKLRNQLLSHRGGEIRS |
| | KDPKNLEDISRKGFSTGLSYMYSSLSEVSQWCKTVDYLGNKISSSIFLYYLRGVL |
| | CPICLLFFKFLISFAFTCALIYVFKGGGFVAMAATNGASSSFLESSGEIDALLQTTT |
| | TTRTPENADYEDDNTSVNQEFLEDGQRARQAKLRELERLILQQYKDFIRQKYPW |
| | IPKGDILLPSMKGGVVEDVMEKLELETYSSSDLTDWINHLRANPKTLNFIFKDFV |
| | A |
| 11 | MAATAKTGRKNHQRHHVLPARGRVGAAAVRCSAVSPVTPPSPAPPATPLRPWG |
| | PAEPRKGADILVEALERCGVSDVFAYPGGASMEIHQALTRSPVITNHLFRHEQGE |
| | AFAASGYARASGRVGVCVATSGPGATNLVSALADALLDSVPMVAITGQVPRRM |
| | IGTDAFQETPIVEVTRSITKHNYLVLDVEDIPRVIQEAFFLASSGRPGPVLVDIPKDI |
| | QQQMAVPVWDTSMNLPGYIARLPKPPATELLEQVLRLVGESRRPILYVGGGCSA |
| | SGDELRWFVELTGIPVTTTLMGLGNFPSDDPLSLRMLGMHGTVYANYAVDKAD |
| | LLLAFGVRFDDRVTGKIEAFASRAKIVHIDIDPAEIGKNKQPHVSICADVKLALQG |
| | LNALLQQSTTKTSSDFSAWHNELDQQKREFPLGYKTFGEEIPPQYAIQVLDELTK |
| | GEAIIATGVGQHQMWAAQYYTYKRPRQWLSSAGLGAMGFGLPAAAGASVANP |
| | GVTVVDIDGDGSFLMNIQELALIRIENLPVKVMVLNNQHLGMVVQLEDRFYKA |
| 12 | NRAHTYLGNPECESEIYPDFVTIAKGFNIPAVRVTKKSEVRAAIKKMLETPGPYL |
| | LDIIVPHQEHVLPMIPIGGAFKDMILDGDGRTVY |
| | ATGGCTGCGACtGCCAAGACCGGCCGTAAGAACCAtCAaCGACAtCAtGTCCTT |
| | CCCGCTCGAGGCCGaGTGGGGGCGGCGGCGGTCAGGTGCTCGGCGGTGTCCC |
| | CaGTCACCCCaCCaTCCCCaGCGCCaCCaGCCACtCCaCTCCGaCCaTGGGGGCCa |
| | GCCGAGCCCCGAAGGGCGCGGACATCCTCGTGGAGGCGCTGGAGCGaTGCG |
| | GCGTCAGCGACGTGTTCGCCTAtCCaGGCGGCGCGTCCATGGAGATCCAtCAaG |
| | CGCTGACtCGtTCCCCaGTCATCACCAACCAtCTCTTCCGtCAtGAGCAaGGCGAG |
| | GCGTTCGCGGCGTCCGGGTAtGCGCGtGCGTCCGGCCGtGTCGGGGTCTGCGTC |
| | GCCACCTCCGGCCCCGGGGCAACCAACCTCGTGTCCGCGCTCGCCGACGCGC |
| | TGCTCGACTCCGTCCCaATGGTCGCCATCACtGGCCAaGTCCCCCGtCGtATGAT |
| | CGGCACCGACGCCTTCCAaGAGACtCCCATAGTCGAGGTCACCCGtTCCATCAC |
| | CAAGCAtAATTAtCTTGTCCTTGATGTGGAGGACATCCCCCGtGTCATACAaGA |
| | AGCCTTCTTCCTCGCGTCCTCGGGCCGTCCTGGCCCaGTGCTGGTCGACATCC |
| | CCAAGGACATCCAaCAaCAaATGGCCGTGCCaGTCTGGGACACCTCGATGAAT |
| | CTACCAGGGTAtATCGCACGtCTGCCCAAGCCACCCGCGACAGAATTGCTTGA |
| | GCAaGTCTTGCGTCTGGTTGGCGAGTCACGaCGtCCaATTCTCTATGTCGGTGG |
| | TGGCTGCTCTGCATCTGGTGACGAATTGCGUTGGTTTGTTGAGCTGACTGGTA |
| | TCCCAGTTACAACCACTCTGATGGGCCTCGGCAATTTCCCCAGTGACGACCCa |
| | TTGTCCCTGCGtATGCTTGGGATGCATGGCACtGTGTAtGCAAATTATGCCGTG |
| | GATAAGGCTGACCTGTTGCTTGCGTTTGGTGTGCGaTTTGATGATCGTGTGAC |
| | AGGGAAAATTGAGGCTTTTGCAAGCAGGGCCAAGATTGTGCAtATTGACATT |
| | GATCCAGCAGAGATTGGAAAGAACAAGCAACCACATGTGTCAATTTGCGCA |
| | GATGTTAAGCTTGCTTTACAaGGCTTGAATGCTCTGCTACAACAaAGCACAAC |
| | AAAGACAAGTTCTGATTTTAGTGCATGGCAtAATGAGTTGGACCAaCAaAAGA |
| | GGGAGTTTCCTCTGGGGTAtAAAACTTTTGGTGAAGAGATCCCACCaCAATAT |
| | GCCATTCAaGTGCTGGATGAGCTGACtAAAGGTGAGGCAATCATCGCTACTGG |
| | TGTTGGGCAaCAtCAaATGTGGGCGGCACAATATTAtACCTAtAAGCGaCCACGa |
| | CAaTGGCTGTCTTCGGCTGGTCTGGGCGCAATGGGATTTGGGCTGCCTGCTGC |
| | AGCTGGTGCTTCTGTGGCTAACCCAGGTGTCACAGTTGTTGATATTGATGGG |
| | GATGGTAGCTTCCTCATGAACATTCAaGAGCTGGCATTGATCCGtATTGAGAA |
| | CCTCCCTGTGAAGGTGATGGTGTTGAACAACCAACATTTGGGTATGGTGGTG |
| | CAATTGGAGGATAGGTTTTAtAAGGCGAATAGGGCGCATACATAtTTGGGCAA |
| | CCCaGAATGTGAGAGCGAGATATATCCAGATTTTGTGACTATTGCTAAGGGG |
| | TTCAATATTCCTGCAGTCCGTGTAACAAAGAAGAGTGAAGTCCGTGCCGCCA |
| | TCAAGAAGATGCTCGAGACTCCAGGGCCATATTGTTGGATATCATCGTCCCa |
| | CAtCAaGAGCATGTGCTGCCTATGATCCCAATTGGGGGCGCATTCAAGGACAT |
| | GATCCTGGATGGTGATGGCAGGACTGTGTATTAA |
| 13 | MFKQASRLLSRSVAAASSKSVTTRAFSTELPSTLDSGAAAPSPATGGVAPVPPSP |
| | RGSIIKRHTLSVFVGDESGMINRIAGVFARRGYNIESLAVGLNKDKALFTIVVSGT |
| | EKILKQVVEQLNKLVNVIQVDDLSKEPQVERELMLIKLNVEPDKRPEVMGLVDI |
| | FRAKVVDLSDHTLTIEVTGDPGKIVAVQRNLSKFGIKEIARTGKIALRREKMGES |
| | APFWRFSAASYPDLEVAMPSKSHVNTAMKTANQNSEESSQGDVYPVESYENFT |
| | TNQILDAHWGVMADGDPTGLCSHSLSILVNDFPGVLNVVTGVFSRRGYNIQSLA |
| | VGPAEKEGTSRITTVVPGTDESIAKLVHQLYKLIDVYEVQDLTHLPFAARELMIIK |
| | IAVNTTARRAILDIADIFRAKTVDVSDHTVTLQLTGDLDKMVALQRMLEPYGICE |
| | VARTGRVALRRESGVDSKYLRGFSLPL |
| 14 | AKAEEIVLQPIREISGAVQLPGSKSLSNRILLLSALSEGTTVVDNLLNSEDVHYML |
| | EALKALGLSVEADKVAKRAVVVGCGGKFPVEKDAKEEVQLFLGNAGIAMRSLT |
| | AAVTAAGGNATYVLDGVPRMRERPIGDLVVGLKQLGADVDCFLGTECPPVRVK |
| | GIGGLPGGKVKLSGSISSQYLSALLMAAPLALGDVEIEIIDKLISIPYVEMTLRLME |
| | RFGVKAEHSDSWDRFYIKGGQKYKSPGNAYVEGDASSASYFLAGAAITGGTVT |
| | VQGCGTTSLQGDVKFAEVLEMMGAKVTWTDTSVTVTGPPREPYGKKHLKAVD |
| | VNMNKMPDVAMTLAVVALFADGPTAIRDVASWRVKETERMVAIRTELTKLGA |
| | SVEEGPDYCIITPPEKLNITAIDTYDDHRMAMAFSLAACADVPVTIRDPGCTRKTF |
| | PNYFDVLSTFVRN |
| 15 | GCGAAGGCGGAGGAGATCGTGCTCCAaCCCATCAGaGAGATtTCCGGGGCGGT |
| | TCAaCTGCCAGGGTCCAAGTCGCTCTCCAACAGaATCCTCCTCCTCTCCGCCCT |
| | CTCCGAGGGCACAACtGTGGTGGACAACTTGCTGAACAGTGAGGATGTTCAtT |
| | AtATGCTTGAGGCCCTGAAAGCCCTCGGGCTCTCTGTGGAAGCAGATAAAGTT |
| | GCAAAgAGAGCTGTAGTCGTTGGCTGTGGTGGCAAGTTTCCTGTTGAGAAGG |
| | ATGCGAAAGAGGAAGTGCAACTCTTCTTGGGGAACGCTGGAAtTGCAATGCG |
| | AtCATTGACtGCtGCCGTGACTGCTGCTGGTGGAAATGCAACTTATGTGCTTGA |
| | TGGAGTGCCACGAATGAGaGAGcgtCCtATTGGTGAtTTGGTTGTCGGGTTGAAA |
| | CAACTTGGTGCGGATGTUGACTGTTTCCTTGGCACTGAATGCCCACCTGTTCG |
| | TGTCAAGGGAATTGGAGGACTTCCTGGTGGCAAGGTTAAGCTCTCTGGTTCC |
| | ATCAGCAGTCAaTAtTTGAGTGCCTTGCTGATGGCTGCTCCTTTGGCCCTTGGG |
| | GATGTGGAGATCGAAATCATTGACAAACTAATCTCCATTCCTTAtGTTGAAAT |
| | GACTTGAGATTGATGGAGCGTTTTGGTGTGAAGGCAGAGCATTCTGATAGTT |
| | GGGACAGATTCTATATTAAGGGAGGGCAaAAGTAtAAATCTCCTGGAAATGC |
| | CTATGTTGAAGGTGATGCCTCAAGCGCGAGCTATTTCTTGGCTGGTGCTGCA |
| | ATCACTGGAGGCACTGTGACAGTTCAAGGTTGTGGTACaACCAGTTTGCAaGG |
| | TGATGTCAAATTTGCTGAGGTACTTGAGATGATGGGAGCAAAGGTTACATGG |
| | ACTGACACCAGTGTAACaGTAACTGGTCCACCtagaGAGCCTTATGGGAAGAAA |
| | CAtCTGAAAGCTGTTGATGTCAACATGAACAAAATGCCTGATGTTGCCATGAC |
| | CCTTGCCGTTGTTGCACTCTTCGCTGATGGTCCAACTGCTATCAGAGATGTGG |
| | CTTCCTGGAGAGTAAAGGAAACtGAAAGaATGGTTGCAATTCGaACtGAGCTA |
| | ACAAAaCTtGGAGCATCGGTTGAAGAAGGTCCTGACTAtTGCATCATCACtCCA |
| | CCtGAGAAGCTGAACATtACtGCAATCGACACtTAtGATGAcCAtAGaATGGCtAT |
| | GGCCTTCTCCCTCGCTGCCTGCGCCGACGTGCCCGTGACtATCcgtGACCCTGG |
| | TTGCACtagaAAGACaTTCCCCAACTAtTTCGACGTTCTAAGCACTTTCGTCAGaA |
| | ACTGA |
| 16 | MSSSLLTDLVNLDLSESTDKVIAEYIWVGGTGMDVRSKARTLSGPVDDPSKLPK |
| | WNFDGSGTGQATGDDSEVILHPQAIFRDPFRKGKNILVMCDCYAPNGEPIPTNN |
| | RYNAARIFSHPDVKAEEPWYGIEQEYTLLQKHINWPLGWPLGGYPGPQGPYYCA |
| | AGADKSYGRDIVDAHYKACLFAGINISGINAEVMPGQWEFQIGPVVGVSAGDHV |
| | WVARYILERITEIAGVVVSFDPKPIPGDWNGAGAHTNYSTKSMRSNGGYEVIKK |
| | AIKKLGMRHREHIAAYGDGNERRLTGRHETADINNFVWGVANRGASVRVGRDT |
| | EKDGKGYFEDRRPASNMDPYLVTAMIAETTILWEPSHGHGHGQSNGK |
| 17 | TCGTCGTCCCTGCTCACTGACCTCGTTAACCTCGACCTGTCGGAGAGCACGA |
| | CAAGGTCATCGCCGAGTAtATATGGGTTGGTGGTACTGGGATGGATGTGAGaA |
| | GCAAAGCCAGAACaTTGTCTGGACCTGTTGATGACCCAAGCAAGCTaCCAAA |
| | GTGGAACTTTGATGGCTCCgGCACtGGTCAaGCTACtGGTGACGACAGTGAAGT |
| | tATCCTCCAtCCTCAAGCCATCTTCcgtGACCCATTCAGaAAGGGGAAGAACATC |
| | CTGGTCATGTGTGACTGTTATGCGCCtAATGGCGAGCCaATTCCtACtAACAAC |
| | CGaTAtAATGCAGCAAGaATCTTCAGTCATCCTGATGTCAAGGCTGAAGAaCCt |
| | TGGTATGGGATTGAGCAaGAGTAtACCCTTCTTCAaAAGCAtATCAACTGGCCT |
| | CTTGGCTGGCCACTAGGTGGCTATCCAGGCCCTCAaGGTCCaTAtTAtTGTGCG |
| | GCGGGAGCCGATAAATCGTAtGGGCGtGACATCGTTGATGCCCATAtAAGGCC |
| | TGCCTGTTTGCCGGCATCAACATCAGCGGGATCAACGCAGAAGTCATGCCtG |
| | GGCAaTGGGAGTTCCAaATTGGCCCTGTCGTTGGCGTaTCCGCAGGGGATCAT |
| | GTCTGGGTGGCACGTAtATTCTTGAGAGaATCACTGAGATTGCTGGCGTCGTC |
| | GTGTCCTTCGACCCCAAGCCCATTCCtGGAGACTGGAATGGtGCCGGTGCTCAt |
| | ACCAATTAtAGCACCAAGTCGATGAGaAGCAATGGCGGCTACGAGGTGATCAA |
| | GAAAGCGATCAAGAAGCTcGGtATGCGCCAtCGTGAGCAtATCGCCGCCTAGG |
| | CGACGGCAACGAGCGtagaCTCACtGGtCGtCAtGAGACtGCCGACATCAACAACT |
| | TCGTCTGGGGCGTAGCGAACCGtGGCGCGTCGGTGCGTGTCGGtCGaGACACC |
| | GAGAAGGACGGCAAAGGTTACTTCGAGGACAGacgaCCaGCGTCCAACATGGA |
| | CCCaTAtCTGGTGACCGCCATGATCGCCGAGACtACCATCCTCTGGGAGCCCAG |
| | tCAtGGtCAtGGaCAtGGCCAATCCAACGGCAAGTGA |
| 18 | gaatttctctgacattccatgtttccgaaaCggatcctata |
| 19 | tgaacagtcactcacttttgacagttataCgattccagaa |
| 20 | GAATTTCTCTGTGAACAGTCACTCACTTTTGACAGTTATACGATTCCAGAAGA |
| | TGATCCAGAATTGGATCCTATA |
| 21 | DEVDFQGPCNDSSDPLVVAASIIGILHLILWILDRL |
| 22 | DEVD |
| 23 | ACUUUUGACAGUUAUACGAUUCCAGAA |
| 24 | UGGGCUGUACCUUCCUCAGGUGUCAAA |
| 25 | GCUGUACCUGGUCGUUCAAAUCUUACC |
| 26 | GGGCCCAAATTCAATTGTATATGAGCTCATATACAAGACCTCACTAGTAAGG |
| | AAGGCACTTGCTGCCGGAGTTCAACAGGCAAATATAAGAAAAGAAGTCCTG |
| | TTCACTTCATCATCTGTGGGTTGTACTGCTTGAAGGTTCTTCTGAGGGGTAGA |
| | ATTTGAATTCCTTCTTTGCTTGTGAGATAACCATTTCCAGAAACTCATATATA |
| | GAGAGCGGGTATCGGTGAAAATGGATCTTACCAGGAGTGGCATTGAATAGG |
| | CAGGCTCTGGGATGTAATCTCACTCAAGAGGTCATTTGTTGGCCCCGCCTTCA |
| | CTAGACTAGAGTTTTAGGATAGGTTGGGGAACCTATACGTCAAGCCCCTACG |
| | AAGATTGAGAAAAATCGATGCACATAAGCCATCCGAAACCAGTATTGGAAA |
| | GTGTTCAGTTTCGTTTTCCATTCTGAAATGTTCATAGTAGTATAGTATGTTTTC |
| | CGTTGGGTCGACGCCATGTGATCGCTACTAAAGATAGAGTTTCCTTGGAAAA |
| | ACCGAGGCCAGTTGAGATCAGTCTCCCTTTCTAGGAGCAGAGCTTAAAAAGA |
| | TGGGAAATTCCAATGAATTTCGATCACAATCATGTGGTAATAATGGGTTTGA |
| | ATCAGAGAGACTCGATCTGGAAACTCCTCAATGATTATAACGTGAACTCGTT |
| | GAAGAGAAGGAGACAAGCAGAAATAGACGCTTTTTTTGAACCATTTGAGAG |
| | GGCGCAGCGTATCCGTTTCAATAACTGGCAGAACGGAATAGAGTTGTTAGAT |
| | GGGGCTGAATGGAGGAACGGCGATATAGTTATCCCTGGAGGCGGCGGACCA |
| | GTAATTTCAAGCCCCTTGGATCAATTTTTCATTGATCCATTATTTGGTCTTGAT |
| | ATGGGTAACTTTTATTTATCATTCACAAATGAATCCTTGTCTATGGCGGTAAC |
| | TGTCGTTTTGGTGCCATCTTTATTTGGAGTTGTTACGAAAAAGGGGGGGGA |
| | AAGTCAGTGCCAAATGCATGGCAATCCTTGGTAGAGCTTATTTATGATTTCGT |
| | GCTGAACCTGGTAAACGAACAAATAGGTGGAAATGTTAAACAAAAGTTTTTC |
| | CCTCGCATCTCGGTCACTTTTACTTTTTCGTTATTTCGTAATCCCCAGGGTATG |
| | ATACCCTTTAGCTTCACAGTGACAAGTCATTTTCTCATTACTTTGGCTCTTTCA |
| | TTTTCCATTTTTATAGGCATTACGATCGTTGGATTTCAAAGACATGGGCTTCA |
| | TTTTTTTAGCTTCTTATTACCAGCGGGAGTCCCACTGCCATTAGCACCTTTTTT |
| | AGTACTCCTTGAGCTAATCTCTCATTGTTTTCGTGCATTAAGCTCAGGAATAC |
| | GTTTATTTGCTAATATGATGGCCGGTCATAGTTCAGTAAAGATTTTAAGTGGG |
| | TTCGCTTGGACTATGCTATTTCTGAATAATATTTTCTATTTCATAGGAGATCTT |
| | GGTCCCTTATTTATTGTATTGGCTCTTACTGGATTGGAATTAGGTGTAGCTAT |
| | ATTACAAGCTCATGTTTCTACGATCTCAATTTGTATTTACTTGAATGATGCTA |
| | TAAATCTCCATCAAAATGAGTAATTTCATAATTGAATAAAAACGAGGAGCCG |
| | AAGATTTTAGGGGGCGGGACAAACGCGGAAGTGTATTGCGTTACAAAAAAT |
| | GACAACTAGCATTTGTTTTTTCATTTCATGTTCGAATTCGTTTTTCGTTGGAAA |
| | AACCAACGCCGACCCCAAACAAGTCTCTCCAATATAAGGAGAGCGGAGCTT |
| | AAAAATATTATTTTATTGTGCTATGGCAAATCTGGTCCGATGGCTCTTCTCCA |
| | CTACCCGAGGGACTAACGGTCTTCCATATTTCATCTTCGGTGTCGTTGTAGGA |
| | GGCGCCCTGTTGTTTGCTTTGCTAAAGTATCAGGCCCCTCTGTACGACCCGGC |
| | TTTAATGGAAAAAATCATAGATCATAATATAAAAGCCGGGCACCCTATAGAG |
| | GTTGACTATTCGTGGTGGGGCACCTCTATTCGTGTAGTCTTTCCTAAGTAAGA |
| | AAGACAGGACAGTGGTGGTTTGCTCATACTTTCATTACAAAACCATACTATG |
| | GAATTAGGGATAACAGGGTAATAATCACAAGTGAGAACCACAGGTAGCAAT |
| | AGGTATTACAGAAATTTCCTCGAGTCTGCTTGAAAGCCTGCAGAGTCCAATT |
| | TTGAGTATTTTCAGTTAGAATCTAGAGTCAGCCTATTCAGTTCTTAGCCCTTA |
| | AGGGTAAGGCAGGGGGTAATATGGATAGTCTCTGTCCCTGTATTCACATTCC |
| | ACCTTCAACAAAGTGTTGATTTCCCGTAAAGCTAACTGTAGTCCTTTAAGTAA |
| | GTAGATATCTTAGGCAAGTTAGCAATCTCGTTATATTACCAAGGCCTTCCCTT |
| | CTATTGTAGAAAGAGTTCTCAGCCATCTAATTGCAGTGCCAGTTGCCAGCTAT |
| | CCAGTTTCATTTGAAGTTGCTGGGGGTCCAAACGAGCTAGTTGCTTTTATTCG |
| | TCCTATAAGTCCTTCCACAAGCGAGTCAATAGGGTGCTGGCTAGTTGTAGTT |
| | GTTGGCGTGCCTTTCCTTTCATCTTGAATATTAATAAATATTTGGATAAATTA |
| | CTTTAGAATAAGAAGTTCATGTTTTAATACGACTCACTATAGTAAGTAATAC |
| | GAATCCATACTAGGAAAATGAAAATGTGAGTCCTAGGCACTGGAATTGGTTC |
| | TCTTCTCCCTAATCCCTATAAGCCAGAAAGGGTAATAGGCTTCAGTGTAAGC |
| | ATTTCCTTCAAGCAAGTCATCTCAAGTTTTAAATTCTAGAGAATAGCTCCGAT |
| | CAACCCATTTTAGTTTGGTTCTGCAATTCATTCGCATAAATGAAAAAAAAAG |
| | CGAGATGTGCACGAAAGAAGATCATAGTTCAGCTTTAAAATGGTGGTGTCCC |
| | TGTGTTAGTAAGTGGTTGAAATAGCTCATGGGAGTGTCTGCCCCATTCGATA |
| | ATGGCATTTATGATCTAGTGGAGTGAGTGATTGTGTGGTGTTCAGTCTAAGG |
| | CTTTTTGAAAAGCGGATTTCTCCCTTCTCTCATCCATCGTCTTTGTTAAAGTTG |
| | AACAGTCACTCACTTTTGACAGTTATAcGATTCCAGAAAAGACCGGCCGTAA |
| | GAACCATCAACGACATCATGTCCTTCCCGCACGAGGCCGAGTGGGGGCG |
| | GCGGCGGTCAGGTGCTCGGCGGTGTCCCCAGTCACCCCACCATCCCCAG |
| | CGCCACCAGCCACTCCACTCCGACCATGGGGGCCAGCCGAGCCCCGTAA |
| | GGGCGCGGACATCCTCGTGGAGGCGCTGGAGCGATGCGGCGTCAGCGA |
| | CGTGTTCGCCTATCCAGGCGGCGCGTCCATGGAGATCCATCAAGCGCTG |
| | ACTCGTTCCCCAGTCATCACCAACCATCTCTTCCGTCATGAGCAAGGCG |
| | AGGCGTTCGCGGCGTCCGGGTATGCGCGTGCGTCCGGCCGTGTCGGGG |
| | TCTGCGTCGCCACCTCCGGCCCCGGGGCAACCAACCTCGTGTCCGCGCT |
| | CGCCGACGCGCTGCTCGACTCCGTCCCAATGGTCGCCATCACTGGCCAA |
| | GTCCCCCGTCGTATGATCGGCACCGACGCCTTCCAAGAGACTCCCATAG |
| | TCGAGGTCACCCGTTCCATCACCAAGCATAATTATCTTGTCCTTGATGTG |
| | GAGGACATCCCCCGTGTCATACAAGAAGCCTTCTTCCTCGCGTCCTCGG |
| | GCCGTCCTGGCCCAGTGCTGGTCGACATCCCCAAGGACATCCAACAACA |
| | AATGGCCGTGCCAGTCTGGGACACCTCGATGAATCTACCAGGGTATATC |
| | GCACGTCTGCCCAAGCCACCCGCGACAGAATTGCTTGAGCAAGTCTTGC |
| | GTCTGGTTGGCGAGTCACGACGTCCAATTCTCTATGTCGGTGGTGGCTG |
| | CTCTGCATCTGGTGACGAATTGCGTTGGTTTGTTGAGCTGACTGGTATC |
| | CCAGTTACAACCACTCTGATGGGCCTCGGCAATTTCCCCAGTGACGACC |
| | CATTGTCCCTGCGTATGCTTGGGATGCATGGCACTGTGTATGCAAATTA |
| | TGCCGTGGATAAGGCTGACCTGTTGCTTGCGTTTGGTGTGCGATTTGAT |
| | GATCGTGTGACAGGGAAAATTGAGGCTTTTGCAAGCAGGGCCAAGATTG |
| | TGCATATTGACATTGATCCAGCAGAGATTGGAAAGAACAAGCAACCACA |
| | TGTGTCAATTTGCGCAGATGTTAAACTTGCTTTACAAGGCTTGAATGCTC |
| | TGCTACAACAAAGCACAACAAAGACAAGTTCTGATTTTAGTGCATGGCA |
| | TAATGAGTTGGACCAACAAAAGAGGGAGTTTCCTCTGGGGTATAAAACT |
| | TTTGGTGAAGAGATCCCACCACAATATGCCATTCAAGTGCTGGATGAGC |
| | TGACTAAAGGTGAGGCAATCATCGCTACTGGTGTTGGGCAACATCAAAT |
| | GTGGGCGGCACAATATTATACCTATAAGCGACCACGACAATGGCTGTCT |
| | TCGGCTGGTCTGGGCGCAATGGGATTTGGGCTGCCTGCTGCAGCTGGTG |
| | CTTCTGTGGCTAACCCAGGTGTCACAGTTGTTGATATTGATGGGGATGG |
| | TAGCTTCCTCATGAACATTCAAGAGCTGGCATTGATCCGTATTGAGAAC |
| | CTCCCTGTGAAGGTGATGGTGTTGAACAACCAACATTTGGGTATGGTGG |
| | TGCAATTGGAGGATAGGTTTTATAAGGCGAATAGGGCGCATACATATTT |
| | GGGCAACCCAGAATGTGAGAGCGAGATATATCCAGATTTTGTGACTATT |
| | GCTAAGGGGTTCAATATTCCTGCAGTCCGTGTAACAAAGAAGAGTGAAG |
| | TCCGTGCCGCCATCAAGAAGATGCTTGAGACTCCAGGGCCATATTTGTT |
| | GGATATCATCGTCCCACATCAAGAGCATGTGCTGCCTATGATCCCAATT |
| | GGGGGCGCATTCAAGGACATGATCCTGGATGGTGATGGCAGGACTGTG |
| | TATTAATGAGGTACCAAGCGATCGCAAACCTAGGAAAAGATCTAGACGAGG |
| | TGTAGCGCAGTCTGGTCAGCGCATCTGTTTTGGGTACAGAGGGCCATAGGTT |
| | CGAATCCTGTCACCTTGAGTCTGGCCCCAAATTCTAATTTCTACTGTTGTAGA |
| | TTTCAATTATGAAATTACTCATGTCCCTTTCGTCCAGTGGTTAGGACATCGTC |
| | TTTTCATGTCGAAGACACGGGTTCGATTCCCGTAAGGGATAGTCTGGCCCCA |
| | AATTCTAATTTCTACTGTTGTAGATGTCACATTTCTACCGGTGCACATTCCAG |
| | CTTATTTGATACCCACTTCAAGTTTCTATCAAACCATGTCTTTTTCTTCGAACG |
| | TCAATCTCGTAGGAAAGATCTAAAAAGCTTAAGCGGCCGCAAAAACCCCTTG |
| | GGGCCTCTAAACGGGTCTTGAGGGGTTTTTTGTAGATAGACCTTTTTATTTTT |
| | CGTCATTCGATCACGAAAAGGCCGGCCAAACCTCGAGGTAATTAAAGCGGCC |
| | GAAATTAGCTAGCAAATAAGCATGCAATCACAAGTGAGAACCACAGGTAGC |
| | AATAGGTATTACAGTAGGGATAACAGGGTAATGAATTCGAAACCATCTTCTC |
| | ACTCTGACCCCCACATATCAGATCCCAGATGCATAGGAAAAGCGGTATCAAG |
| | AATAGTAGTATAAAGAAAGATAGTACAGTACTCAAGTAAATGAATTCGCCTA |
| | AGGATCGATGGAAAGATCAAGGTCCCCGTGAAAAAGTAGATACTAGATCGA |
| | TATGATACTCTCATCTCTGGAGTAACTTCTTCCATTATGCTGATCTCTAGGTC |
| | CGTTCCATCATCATCGTAATAGTATGGTCCCAGGTGTCCGAGCTATAGATCA |
| | AGATCATATCCAGTCACATTTCTACCGGTGCACTTCTCATGAAATAATTCCCT |
| | TTCCAAGGAAAGGAAAACAAGAACTCGAATACTCGTAATAGCGATCCCGAT |
| | CCACCTACTTTTTTCTATTCTTTGATTCGAAACGTGCTAAAGCACAAGCCATT |
| | TTTATGCATGGGGCATAAGAGTGGACAATCTATGTTATCGAAGGAAGTAAAT |
| | AACAACACTTCAGCGTTTAGGTCTACCTTCAGTAAACCAATAGTTTTGCAGC |
| | ATTGGAATTTGAGTTGGCCAGGTAAGGTCCTCTAAAAAGAAAAGAAGAAAC |
| | TACTTAGAATAGATAAATGCCATTGGTTTTCTCGTACTATACGATCTTTTTTT |
| | GTTTTGTTTTTTGGCCATGATTGTGCTGCTCCTGTGAAGGCTAGTGGGAAAGC |
| | TCACCGTTCGTTGTGATGAGTGGGGGCCTTGTATCTGTATTCGGATCAGCTCC |
| | TTAACAGAGTTTCCTGCTTGAACCCTGGCTGGGAGCTGGGAGAGGTGTCCCA |
| | CTACAGGTGCAAATAAACCATTTGACCTTACAGGGGAAAGGAAACAAACCA |
| | CTCAATAATCGGTAGAAATTCCTCCTACTGAACAGCTTTCCTTTTCTCGCCTT |
| | AACTACTACTTCAAAGCAAGGCGGAATATCACGGGATAGGAATGAAAGAAC |
| | TTCTTACTCAACTTTCTAGCTATATAAAAATAGTTAGCAATATGAAACGAGTA |
| | ACTTAAGCCCTAGTAAAAGGCTACTCTTTGAATCCCCTCTTTAAGGCATATAA |
| | AATTAGTACTCTTCCTGAGCTAGCTTAAGCATATCTTGAGCGAGTGAGTTGTA |
| | TTTCCCTCCATCAAGTTCTAAGCGATCAAATAAGGTCCTTGCTCTCGAGCCAA |
| | TGCCAATACCAATAGAGAGGGTCTAAACGAAGGATTCAAAGGCGCGCC |
| 27 | AACCCCTTGGGGCCTCTAAACGGGTCTTGAGGGGTTTTTTGtagatagacctttttatttttc |
| | gtcattcgatcacgaaaacagggattctggaacggccaagaatcccagcggttgttcgggtcgaaaaaccgagaacaagacat |
| | gccacaaagtggcagatgaaggcaggggggagagcctagtcctcaacctcttcttccccaaaaggtagttatgaacgtgccaa |
| | acttattggatttattcttggaatgctcataaccacctttactctttttttcattctttactcagaggaagccatgccgtttg |
| | gagaagagcaccaagtggggggagtgtggagtccccgaaagaggagctttctaaaggcaagagaaaagctccgatggagccct |
| | tggagctacagggaccaccaacccttcgcagcttggacgatttgattcttgtgccactcagccctgaggagggcgcctgctcg |
| | acccagtcaggtactactccgccgccggccccgagtaactctgcgggggtcggtgcagccctttcttctattccggagtgcat |
| | aaacaaggatcctcaaaaagcgaaatcatttcgttaatggcatttcagaaatgagtcataggcgcctgtacaatgacagaata |
| | gagagtcctttttttccagaatgaatcattctattcaaatctcacaagttctctttacgcgtcttctaggggcattgttgaac |
| | gcaatctgcaggaacaagaaatgattctttcttattttgaaacagaa |
| 28 | AACCCCTTGGGGCCTCTAAACGGGTCTTGAGGGGTTTTTTGtagatagacctttttatttttc |
| | gtcattcgatcacgaaaa |
| 29 | CAAACGCGGAAGTGTATTGCGTTACAAAAAATGACAACTAGCATTTGTTTTT |
| | TCATTTCATGTTCGAATTCGTTTTTCGTTGGAAAAACCAACGCCGACCCCAAA |
| | CAAGTCTCTCCAATATAAGGAGAGCGGAGCTTAAAAATATTATTTTATTGTG |
| | CTATGGCAAATCTGGTCCGATGGCTCTTCTCCACTACCCGAGGGACTAACGG |
| | TCTTCCATATTTCATCTTCGGTGTCGTTGTAGGAGGCGCCCTGTTGTTTGCTTT |
| | GCTAAAGTATCAGGCCCCTCTGTACGACCCGGCTTTAATGGAAAAAATCATA |
| | GATCATAATATAAAAGCCGGGCACCCTATAGAGGTTGACTATTCGTGGTGGG |
| | GCACCTCTATTCGTGTAGTCTTTCCTAAGTAAGAAAGACAGGACAGTGGTGG |
| | TTTGCTCATACTTTCATTACAAAACCATACTATGGAAT |
| 30 | agatctagacgaggtgtagcgcagtctggtcagcgcatctgttttgggtacagagggccataggttcgaatcctgtcaccttg |
| | aGTCTGGCCCCAAATTCTAATTTCTACTGTTGTAGATTTCAATTATGAAATTACT |
| | CATgtccctttcgtccagtggttaggacatcgtcttttcatgtcgaagacacgggttcgattcccgtaagggataGTCTGG |
| | CCCCAAATTCTAATTTCTACTGTTGTAGATGTCACATTTCTACCGGTGCACattc |
| | cagcttatttgatacccacttcaagtttctatcaaaccatgtctttttcttcgaacgtcaatctcgtaggaaagatct |
| 31 | MNNGTNNFQNFIGISSLQKTLRNALIPTETTQQFIVKNGIIKEDELRGENRQILKDI |
| | MDDYYRGFISETLSSIDDIDWTSLFEKMEIQLKNGDNKDTLIKEQTEYRKAIHKK |
| | FANDDRFKNMFSAKLISDILPEFVIHNNNYSASEKEEKTQVIKLFSRFATSFKDYF |
| | KNRANCFSADDISSSSCHRIVNDNAEIFFSNALVYRRIVKSLSNDDINKISGDMKD |
| | SLKEMSLEEIYSYEKYGEFITQEGISFYNDICGKVNSFMNLYCQKNKENKNLYKL |
| | QKLHKQILCIADTSYEVPYKFESDEEVYQSVNGFLDNISSKHIVERLRKIGDNYN |
| | GYNLDKIYIVSKFYESVSQKTYRDWETINTALEIHYNNILPGNGKSKADKVKKA |
| | VKNDLQKSITEINELVSNYKLCSDDNIKAETYIHEISHILNNFEAQELKYNPEIHLV |
| | ESELKASELKNVLDVIMNAFHWCSVFMTEELVDKDNNFYAELEEIYDEIYPVISL |
| | YNLVRNYVTQKPYSTKKIKLNFGIPTLADGWSKSKEYSNNAIILMRDNLYYLGIF |
| | NAKNKPDKKIIEGNTSENKGDYKKMIYNLLPGPNKMIPKVFLSSKTGVETYKPSA |
| | YILEGYKQNKHIKSSKDFDITFCHDLIDYFKNCIAIHPEWKNFGFDFSDTSTYEDIS |
| | GFYREVELQGYKIDWTYISEKDIDLLQEKGQLYLFQIYNKDFSKKSTGNDNLHT |
| | MYLKNLFSEENLKDIVLKLNGEAEIFFRKSSIKNPIIHKKGSILVNRTYEAEEKDQF |
| | GNIQIVRKNIPENIYQELYKYFNDKSDKELSDEAAKLKNVVGHHEAATNIVKDY |
| | RYTYDKYFLHMPITINFKANKTGFINDRILQYIAKEKDLHVIGIDRGERNLIYVSVI |
| | DTCGNIVEQKSFNIVNGYDYQIKLKQQEGARQIARKEWKEIGKIKEIKEGYLSLVI |
| | HEISKMVIKYNAIIAMEDLSYGFKKGRFKVERQVYQKFETMLINKLNYLVFKDIS |
| | ITENGGLLKGYQLTYIPDKLKNVGHQCGCIFYVPAAYTSKIDPTTGFVNIFKFKDL |
| | TVDAKREFIKKFDSIRYDSEKNLFCFTFDYNNFITQNTVMSKSSWSVYTYGVRIK |
| | RRFVNGRFSNESDTIDITKDMEKTLEMTDINWRDGHDLRQDIIDYEIVQHIFEIFR |
| | LTVQMRNSLSELEDRDYDRLISPVLNENNIFYDSAKAGDALPKDADANGAYCIA |
| | LKGLYEIKQITENWKEDGKFSRDKLKISNKDWFDFIQNKRYL |
| 32 | caagacctcactagtaaggaaggcacttgctgccggagttcaacaggcaaatataagaaaagaagtcctgttcacttcatcat |
| | ctgtgggttgtactgcttgaaggttcttctgaggggtagaatttgaattccttctttgcttgtgagataaccatttccagaaa |
| | ctcatatatagagagcgggtatcggtgaaaatggatcttaccaggagtggcattgaataggcaggctctgggatgtaatctca |
| | ctcaagaggtcatttgttggccccgccttcactagactagagttttaggataggttggggaacctatacgtcaagcccctacg |
| | aagattgagaaaaatcgatgcacataagccatccgaaaccagtattggaaagtgttcagtttcgttttccattctgaaatgtt |
| | catagtagtatagtatgttttccgttgggtcgacgccatgtgatcgctactaaagatagagtttccttggaaaaaccgaggcc |
| | agttgagatcagtctccctttctaggagcagagcttaaaaagatgggaaattccaatgaatttcgatcacaatcatgtggtaa |
| | taatgggtttgaatcagagagactcgatctggaaactcctcaatgattataacgtgaactcgttgaagagaaggagacaagca |
| | gaaatagacgctttttttgaaccatttgagagggcgcagcgtatccgtttcaataactggcagaacggaatagagttgttaga |
| | tggggctgaatggaggaacggcgatatagttatccctggaggcggcggaccagtaatttcaagccccttggatcaatttttca |
| | ttgatccattatttggtcttgatatgggtaacttttatttatcattcacaaatgaatccttgtctatggcggtaactgtcgtt |
| | ttggtgccatctttatttggagttgttacgaaaaagggcgggggaaagtcagtgccaaatgcatggcaatccttggtagagct |
| | tatttatgatttcgtgctgaacctggtaaacgaacaaataggtggaaatgttaaacaaaagtttttccctcgcatctcggtca |
| | cttttactttttcgttatttcgtaatccccagggtatgataccctttagcttcacagtgacaagtcattttctcattactttg |
| | gctctttcattttccatttttataggcattacgatcgttggatttcaaagacatgggcttcatttttttagcttcttattacc |
| | agcgggagtcccactgccattagcaccttttttagtactccttgagctaatctctcattgttttcgtgcattaagctcaggaa |
| | tacgtttatttgctaatatgatggccggtcatagttcagtaaagattttaagtgggttcgcttggactatgctatttctgaat |
| | aatattttctatttcataggagatcttggtcccttatttatTgtATtGgcTCTTacTggATtggaattaggtgtagctatatt |
| | acaagctcatgtttctacgatctcaatttgtatttacttgaatgatgctataaatctccatcaaaatgagtaatttcataatt |
| | gaataaaaacgaggagccgaagattttagggggcggga |
| 33 | gaattcgaaaccatcttctcactctgacccccacatatcagatcccagatgcataggaaaagcggtatcaagaatagtagtat |
| | aaagaaagatagtacagtactcaagtaaatgaattcgcctaaggatcgatggaaagatcaaggtccccgtgaaaaagtagata |
| | ctagatcgatatgatactctcatctctggagtaacttcttccattatgctgatctctaggtccgttccatcatcatcgtaata |
| | gtatggtcccaggtgtccgagctatagatcaagatcatatCCagtcacatttctaccggtgcacttctcatgaaataattccc |
| | tttccaaggaaaggaaaacaagaactcgaatactcgtaatagcgatcccgatccacctacttttttctattctttgattcgaa |
| | acgtgctaaagcacaagccatttttatgcatggggcataagagtggacaatctatgttatcgaaggaagtaaataacaacact |
| | tcagcgtttaggtctaccttcagtaaaccaatagttttgcagcattggaatttgagttggccaggtaaggtcctctaaaaaga |
| | aaagaagaaactacttagaatagataaatgccattggttttctcgtactatacgatctttttttgttttgttttttggccatg |
| | attgtgctgctcctgtgaaggctagtgggaaagctcaccgttcgttgtgatgagtgggggccttgtatctgtattcggatcag |
| | ctccttaacagagtttcctgcttgaaccctggctgggagctgggagaggtgtcccactacaggtgcaaataaaccatttgacc |
| | ttacaggggaaaggaaacaaaccactcaataatcggtagaaattcctcctactgaacagctttccttttctcgccttaactac |
| | tacttcaaagcaaggcggaatatcacgggataggaatgaaagaacttcttactcaactttctagctatataaaaatagttagc |
| | aatatgaaacgagtaacttaagccctagtaaaaggctactctttgaatcccctctttaaggcatataaaattagtactcttcc |
| | tgagctagcttaagcatatcttgagcgagtgagttgtatttccctccatcaagttctaagcgatcaaataaggtccttgctct |
| | cgagccaatgccaataccaatagagagggtctaaacgaaggattcaaa |
| 34 | ATGTTTAAACAAGCTTCTCGTCTCCTCTCCCGATCTGTCGCCGCCGCATCTTC |
| | CAAATCGGTGACGACTCGTGCCTTTTCAACGGAACTTCCATCGACGCTCGATT |
| | CCaacacgattaacatcgctaagaacgacttctctgacatcgaactggctgctatcccgttcaacactctggctgaccattac |
| | ggtgagcgtttagctcgcgaacagttggcccttgagcatgagtcttacgagatgggtgaagcacgcttccgcaagatgtttga |
| | gcgtcaacttaaagctggtgaggttgcggataacgctgccgccaagcctctcatcactaccctactccctaagatgattgcac |
| | gcatcaacgactggtttgaggaagtgaaagctaagcgcggcaagcgcccgacagccttccagttcctgcaagaaatcaagccg |
| | gaagccgtagcgtacatcaccattaagaccactctggcttgcctaaccagtgctgacaatacaaccgttcaggctgtagcaag |
| | cgcaatcggtcgggccattgaggacgaggctcgcttcggtcgtatccgtgaccttgaagctaagcacttcaagaaaaacgttg |
| | aggaacaactcaacaagcgcgtagggcacgtctacaagaaagcatttatgcaagttgtcgaggctgacatgctctctaagggt |
| | ctactcggtggcgaggcgtggtcttcgtggcataaggaagactctattcatgtaggagtacgctgcatcgagatgctcattga |
| | gtcaaccggaatggttagcttacaccgccaaaatgctggcgtagtaggtcaagactctgagactatcgaactcgcacctgaat |
| | acgctgaggctatcgcaacccgtgcaggtgcgctggctggcatctctccgatgttccaaccttgcgtagttcctcctaagccg |
| | tggactggcattactggtggtggctattgggctaacggtcgtcgtcctctggcgctggtgcgtactcacagtaagaaagcact |
| | gatgcgctacgaagacgtttacatgcctgaggtgtacaaagcgattaacattgcgcaaaacaccgcatggaaaatcaacaaga |
| | aagtcctagcggtcgccaacgtaatcaccaagtggaagcattgtccggtcgaggacatccctgcgattgagcgtgaagaactc |
| | ccgatgaaaccggaagacatcgacatgaatcctgaggctctcaccgcgtggaaacgtgctgccgctgctgtgtaccgcaagga |
| | caaggctcgcaagtctcgccgtatcagccttgagttcatgcttgagcaagccaataagtttgctaaccataaggccatctggt |
| | tcccttacaacatggactggcgcggtcgtgtttacgctgtgtcaatgttcaacccgcaaggtaacgatatgaccaaaggactg |
| | cttacgctggcgaaaggtaaaccaatcggtaaggaaggttactactggctgaaaatccacggtgcaaactgtgcgggtgtcga |
| | taaggttccgttccctgagcgcatcaagttcattgaggaaaaccacgagaacatcatggcttgcgctaagtctccactggaga |
| | acacttggtgggctgagcaagattctccgttctgcttccttgcgttctgctttgagtacgctggggtacagcaccacggcctg |
| | agctataactgctcccttccgctggcgtttgacgggtcttgctctggcatccagcacttctccgcgatgctccgagatgaggt |
| | aggtggtcgcgcggttaacttgcttcctagtgaaaccgttcaggacatctacgggattgttgctaagaaagtcaacgagattc |
| | tacaagcagacgcaatcaatgggaccgataacgaagtagttaccgtgaccgatgagaacactggtgaaatctctgagaaagtc |
| | aagctgggcactaaggcactggctggtcaatggctggcttacggtgttactcgcagtgtgactaagcgttcagtcatgacgct |
| | ggcttacgggtccaaagagttcggcttccgtcaacaagtgctggaagataccattcagccagctattgattccggcaagggtc |
| | tgatgttcactcagccgaatcaggctgctggatacatggctaagctgatttgggaatctgtgagcgtgacggtggtagctgcg |
| | gttgaagcaatgaactggcttaagtctgctgctaagctgctggctgctgaggtcaaagataagaagactggagagattcttcg |
| | caagcgttgcgctgtgcattgggtaactcctgatggtttccctgtgtggcaggaatacaagaagcctattcagacgcgcttga |
| | acctgatgttcctcggtcagttccgcttacagcctaccattaacaccaacaaagatagcgagattgatgcacacaaacaggag |
| | tctggtatcgctcctaactttgtacacagccaagacggtagccaccttcgtaagactgtagtgtgggcacacgagaagtacgg |
| | aatcgaatcttttgcactgattcacgactccttcggtaccattccggctgacgctgcgaacctgttcaaagcagtgcgcgaaa |
| | ctatggttgacacatatgagtcttgtgatgtactggctgatttctacgaccagttcgctgaccagttgcacgagtctcaattg |
| | gacaaaatgccagcacttccggctaaaggtaacttgaacctccgtgacatcttagagtcggacttcgcgttcgcgtaa |
| 35 | MFKQASRLLSRSVAAASSKSVTTRAFSTELPSTLDSNTINIAKNDFSDIELAAIPFN |
| | TLADHYGERLAREQLALEHESYEMGEARFRKMFERQLKAGEVADNAAAKPLIT |
| | TLLPKMIARINDWFEEVKAKRGKRPTAFQFLQEIKPEAVAYITIKTTLACLTSADN |
| | TTVQAVASAIGRAIEDEARFGRIRDLEAKHFKKNVEEQLNKRVGHVYKKAFMQ |
| | VVEADMLSKGLLGGEAWSSWHKEDSIHVGVRCIEMLIESTGMVSLHRQNAGVV |
| | GQDSETIELAPEYAEAIATRAGALAGISPMFQPCVVPPKPWTGITGGGYWANGR |
| | RPLALVRTHSKKALMRYEDVYMPEVYKAINIAQNTAWKINKKVLAVANVITKW |
| | KHCPVEDIPAIEREELPMKPEDIDMNPEALTAWKRAAAAVYRKDKARKSRRISL |
| | EFMLEQANKFANHKAIWFPYNMDWRGRVYAVSMFNPQGNDMTKGLLTLAKG |
| | KPIGKEGYYWLKIHGANCAGVDKVPFPERIKFIEENHENIMACAKSPLENTWWA |
| | EQDSPFCFLAFCFEYAGVQHHGLSYNCSLPLAFDGSCSGIQHFSAMLRDEVGGR |
| | AVNLLPSETVQDIYGIVAKKVNEILQADAINGTDNEVVTVTDENTGEISEKVKLG |
| | TKALAGQWLAYGVTRSVTKRSVMTLAYGSKEFGFRQQVLEDTIQPAIDSGKGL |
| | MFTQPNQAAGYMAKLIWESVSVTVVAAVEAMNWLKSAAKLLAAEVKDKKTG |
| | EILRKRCAVHWVTPDGFPVWQEYKKPIQTRLNLMFLGQFRLQPTINTNKDSEIDA |
| | HKQESGIAPNFVHSQDGSHLRKTVVWAHEKYGIESFALIHDSFGTIPADAANLFK |
| | AVRETMVDTYESCDVLADFYDQFADQLHESQLDKMPALPAKGNLNLRDILESD |
| | FAFA |
| 36 | MFKQASRLLSRSVAAASSKSVTTRAFSTELPSTLDS |
| 37 | GAAAAGGTCAAGTCTAAGCGACATATGGCACGAAAAGGAAATCCAATTTCG |
| | GTAATCCGAAGCCCACCTTACCTTAATCCATTTTCTTGAGGATCTTGCATTTG |
| | TTCGTTCTCTGCCGACCTATTGTTTCCATCACTCCTGTTGCTTCTTCCTTTCTCT |
| | ATCCAACCCGAGCTCTATCGCCCATGGCTGAACTCTTCTCTGCAGCCTCATCG |
| | CCAACAAAAGGTCCACATCCTTCAATCGGCATGTACTATGGAATGGCTTTAA |
| | TGTTTCTATATCCGGAAGTAGGGAATCTCGTGCTTGCATATCTAAATCTAAGT |
| | TTTGAGACAGACCTTTCATGGGTTCAAAGAAAAGAAGAGTACGAGTGGGTG |
| | ATGTGATTGACTAATAGGAAAGTGATAGGAGTTGTGATCGGGTTTCTTTCTTT |
| | TTTTGCCAAGGATATTCTTATTCACTGCTCAGAGAAAATGAAAAAAAACAAA |
| | CTAGGATTCTTCCGAACTCAACTCCGTGGCCA |
| 38 | CTCATCGCCAACAAAAGG |
| 39 | CCATAGTACATGCCGATT |
| 40 | ATGGCAGCGAAGATCCGGATCGTCATGAAGTCCTTCATGAGCCAGGCCAATA |
| | AGGTGGAGGGCGTCATCCCGTATGCCCAAAAAGTCGGGCTCCCAGAGTCGCG |
| | CTCTCTCTACACAGTCTTGCGGTCGCCACATATCGATAAAAAGTCCAGAGAA |
| | CAATTCTCTATGACCGGCCGCGCCACCATGGGAAAACCTATTCCTAATCCTCT |
| | GCTGGGCCTGGATTCTACCGGAGGCGTGGACCTGAGAACACTGGGATATTCT |
| | CAGCAGCAGCAGGAGAAGATCAAGCCCAAGGTGAGATCTACAGTGGCCCAG |
| | CACCACGAAGCCCTGGTGGGACACGGATTTACACACGCCCACATTGTGGCCC |
| | TGTCTCAGCACCCTGCCGCCCTGGGAACAGTGGCCGTGAAATATCAGGATAT |
| | GATTGCCGCCCTGCCTGAGGCCACACACGAAGCCATTGTGGGAGTGGGAAA |
| | ACGAGGCGCTGGAGCCAGAGCCCTGGAAGCCCTGCTGACAGTGGCCGGAGA |
| | ACTGAGAGGACCTCCTCTGCAGCTGGATACAGGACAGCTGCTGAAGATTGCC |
| | AAAAGGGGCGGAGTGACCGCGGTGGAAGCCGTGCACGCCTGGAGAAATGCC |
| | CTGACAGGAGCCCCTCTGAACCTGACCCCCGAACAGGTGGTGGCCATTGCCA |
| | GCCACGACGGCGGCAAGCAGGCCCTGGAAACCGTGCAGAGACTGCTGCCCG |
| | TGCTGTGCCAGGCCCATGGCCTGACACCTGAACAGGTGGTGGCTATCGCCTC |
| | TAACGGCGGAGGAAAACAGGCTCTGGAAACAGTGCAGCGGCTGCTGCCTGT |
| | GCTGTGTCAGGCTCACGGCTTGACTCCAGAACAGGTGGTGGCTATTGCTTCC |
| | CACGACGGGGGGAAACAGGCCCTGGAAACTGTGCAGCGCCTGCTGCCAGTG |
| | CTGTGCCAGGCTCACGGACTGACCCCCGAACAGGTGGTGGCCATTGCCAGCA |
| | ACATCGGCGGCAAGCAGGCCCTGGAAACCGTGCAGAGACTGCTGCCCGTGCT |
| | GTGCCAGGCCCATGGCCTGACACCTGAACAGGTGGTGGCTATCGCCTCTAAC |
| | GGCGGAGGAAAACAGGCTCTGGAAACAGTGCAGCGGCTGCTGCCTGTGCTG |
| | TGTCAGGCTCACGGCTTGACTCCAGAACAGGTGGTGGCTATTGCTTCCCACG |
| | ACGGGGGGAAACAGGCCCTGGAAACTGTGCAGCGCCTGCTGCCAGTGCTGT |
| | GCCAGGCTCACGGGCTGACCCCCGAACAGGTGGTGGCCATTGCCAGCAACA |
| | ACGGCGGCAAGCAGGCCCTGGAAACCGTGCAGAGACTGCTGCCCGTGCTGT |
| | GCCAGGCCCATGGCCTGACACCTGAACAGGTGGTGGCTATCGCCTCTCACGA |
| | CGGAGGAAAACAGGCTCTGGAAACAGTGCAGCGGCTGCTGCCTGTGCTGTGT |
| | CAGGCTCACGGCTTGACTCCAGAACAGGTGGTGGCTATTGCTTCCCACGACG |
| | GGGGGAAACAGGCCCTGGAAACTGTGCAGCGCCTGCTGCCAGTGCTGTGCCA |
| | GGCTCACGGCCTCACTCCCGAACAGGTGGTGGCCATTGCCAGCAACATCGGC |
| | GGCAAGCAGGCCCTGGAAACCGTGCAGAGACTGCTGCCCGTGCTGTGCCAG |
| | GCCCATGGCCTGACACCTGAACAGGTGGTGGCTATCGCCTCTAATATCGGAG |
| | GAAAACAGGCTCTGGAAACAGTGCAGCGGCTGCTGCCTGTGCTGTGTCAGGC |
| | TCACGGCTTGACTCCAGAACAGGTGGTGGCTATTGCTTCCCACGACGGGGGG |
| | AAACAGGCCCTGGAAACTGTGCAGCGCCTGCTGCCAGTGCTGTGCCAGGCTC |
| | ACGGACTGACCCCCGAACAGGTGGTGGCCATTGCCAGCAACATCGGCGGCA |
| | AGCAGGCCCTGGAAACCGTGCAGAGACTGCTGCCCGTGCTGTGCCAGGCCCA |
| | TGGCCTGACACCTGAACAGGTGGTGGCTATCGCCTCTAATATCGGAGGAAAA |
| | CAAGCACTCGAGACAGTGCAGCGGCTGCTGCCTGTGCTGTGTCAGGCTCACG |
| | GCTTGACTCCAGAACAGGTGGTGGCTATTGCTTCCAATATTGGGGGGAAACA |
| | GGCCCTGGAAACTGTGCAGCGCCTGCTGCCAGTGCTGTGCCAGGCTCACGGC |
| | CTGACCCCCGAACAGGTGGTGGCCATTGCCAGCAACATCGGCGGCAAGCAG |
| | GCCCTGGAAACCGTGCAGAGACTGCTGCCCGTGCTGTGCCAGGCCCATGGCC |
| | TGACACCTGAACAGGTGGTGGCTATCGCCTCTAACAACGGAGGAAAACAGG |
| | CTCTGGAAACAGTGCAGCGGCTGCTGCCTGTGCTGTGTCAGGCTCACGGCTT |
| | GACTCCACAGCAGGTCGTGGCAATTGCTAGCAACAACGGCGGACGGCCCGC |
| | CCTGGAGAGCATTGTGGCCCAGCTGTCTAGACCGGACCCAGCTCTCGCTGCA |
| | TTGACTAACGACCATCTCGTCGCTTTGGCTTGCCTCGGTGGAAGGCCAGCTCT |
| | TGACGCTGTAAAGAAAGGTTTGCCACATGCTCCAGCTCTTATTAAAAGGACT |
| | GGTAGCGGTAGCCAACTCGTAAAGAGCGAATTGGAGGAAAAGAAAAGCGAA |
| | TTGAGGCATAAACTAAAATATGTACCACATGAATATATTGAACTCATTGAAA |
| | TTGCTAGGAACTCAACTCAAGACAGGATTCTTGAAATGAAAGTTATGGAATT |
| | TTTCATGAAAGTCTATGGTTACAGGGGGAAACATCTTGGTGGGTCAAGGAAG |
| | CCAGATGGGGCTATCTACACGGTCGGGTCACCTATTGACTACGGAGTAATTG |
| | TGGACACCAAAGCATATTCTGGTGGGTATAACCTCCCAATCGGGCAAGCAGA |
| | CGAAATGCAAAGGTATGTAGAAGAAAATCAAACTAGAAATAAACATATTAA |
| | TCCCAATGAATGGTGGAAAGTATATCCAAGCTCTGTAACTGAATTTAAATTC |
| | TTGTTCGTATCTGGTCATTTCAAAGGAAACTACAAAGCTCAACTTACTAGACT |
| | AAATCATATTACCAACTGCAACGGTGCCGTTCTCAGCGTAGAAGAATTGCTC |
| | ATCGGTGGGGAAATGATCAAAGCTGGCACGCTTACGCTTGAGGAAGTAAGG |
| | AGAAAATTCAATAACGGAGAAATTAACTTTGGCAGCTGA |
| 41 | MAAKIRIVMKSFMSQANKVEGVIPYAQKVGLPESRSLYTVLRSPHIDKKSREQFS |
| | MTGRATMGKPIPNPLLGLDSTGGVDLRTLGYSQQQQEKIKPKVRSTVAQHHEAL |
| | VGHGFTHAHIVALSQHPAALGTVAVKYQDMIAALPEATHEAIVGVGKRGAGAR |
| | ALEALLTVAGELRGPPLQLDTGQLLKIAKRGGVTAVEAVHAWRNALTGAPLNL |
| | TPEQVVAIASHDGGKQALETVQRLLPVLCQAHGLTPEQVVAIASNGGGKQALET |
| | VQRLLPVLCQAHGLTPEQVVAIASHDGGKQALETVQRLLPVLCQAHGLTPEQV |
| | VAIASNIGGKQALETVQRLLPVLCQAHGLTPEQVVAIASNGGGKQALETVQRLL |
| | PVLCQAHGLTPEQVVAIASHDGGKQALETVQRLLPVLCQAHGLTPEQVVAIASN |
| | NGGKQALETVQRLLPVLCQAHGLTPEQVVAIASHDGGKQALETVQRLLPVLCQ |
| | AHGLTPEQVVAIASHDGGKQALETVQRLLPVLCQAHGLTPEQVVAIASNIGGKQ |
| | ALETVQRLLPVLCQAHGLTPEQVVAIASNIGGKQALETVQRLLPVLCQAHGLTP |
| | EQVVAIASHDGGKQALETVQRLLPVLCQAHGLTPEQVVAIASNIGGKQALETVQ |
| | RLLPVLCQAHGLTPEQVVAIASNIGGKQALETVQRLLPVLCQAHGLTPEQVVAIA |
| | SNIGGKQALETVQRLLPVLCQAHGLTPEQVVAIASNIGGKQALETVQRLLPVLCQ |
| | AHGLTPEQVVAIASNNGGKQALETVQRLLPVLCQAHGLTPQQVVAIASNNGGRP |
| | ALESIVAQLSRPDPALAALTNDHLVALACLGGRPALDAVKKGLPHAPALIKRTG |
| | SGSQLVKSELEEKKSELRHKLKYVPHEYIELIEIARNSTQDRILEMKVMEFFMKV |
| | YGYRGKHLGGSRKPDGAIYTVGSPIDYGVIVDTKAYSGGYNLPIGQADEMQRY |
| | VEENQTRNKHINPNEWWKVYPSSVTEFKFLFVSGHFKGNYKAQLTRLNHITNCN |
| | GAVLSVEELLIGGEMIKAGTLTLEEVRRKENNGEINFGS |
| 42 | MAAKIRIVMKSFMSQANKVEGVIPYAQKVGLPESRSLYTVLRSPHIDKKSREQFS |
| | M |
| 43 | ATGTTTAAACAAGCTTCCCGTCTTCTGTCACGCTCAGTTGCAGCTGCTTCCTC |
| | TAAGTCCGTCACCACCCGCGCTTTCTCCACCGAGCTGCCATCAACCCTGGACT |
| | CCACCGGCCGCGCCACCATGGGAAAACCGATCCCAAACCCGCTTTTGGGGCT |
| | CGACAGCACAGGAGGGGTTGATCTCAGGACCCTCGGTTACAGCCAACAGCA |
| | ACAAGAAAAAATTAAGCCTAAAGTGAGGTCCACGGTAGCTCAACATCACGA |
| | GGCTTTGGTAGGGCATGGCTTCACGCACGCTCATATCGTCGCTCTCAGCCAA |
| | CATCCCGCTGCGCTTGGGACGGTCGCTGTTAAGTACCAAGACATGATCGCTG |
| | CGCTTCCGGAAGCTACGCATGAGGCGATCGTCGGTGTAGGGAAGCGGGGAG |
| | CAGGTGCGAGGGCGCTCGAGGCGCTCTTGACCGTCGCGGGTGAGCTTAGGGG |
| | TCCGCCACTCCAATTGGACACCGGACAACTTTTGAAAATCGCTAAGAGAGGA |
| | GGCGTCACAGCCGTCGAGGCGGTGCACGCCTGGAGAAATGCCCTGACAGGA |
| | GCCCCTCTGAACCTGACCCCCGAACAGGTGGTGGCCATTGCCAGCCACGACG |
| | GCGGCAAGCAGGCCCTGGAAACCGTGCAGAGACTGCTGCCCGTGCTGTGCCA |
| | GGCCCATGGCCTGACACCTGAACAGGTGGTGGCTATCGCCTCTCACGACGGA |
| | GGAAAACAGGCTCTGGAAACAGTGCAGCGGCTGCTGCCTGTGCTGTGTCAGG |
| | CTCACGGCTTGACTCCAGAACAGGTGGTGGCTATTGCTTCCAATATTGGGGG |
| | GAAACAGGCCCTGGAAACTGTGCAGCGCCTGCTGCCAGTGCTGTGCCAGGCT |
| | CACGGACTGACCCCCGAACAGGTGGTGGCCATTGCCAGCAACGGCGGCGGC |
| | AAGCAGGCCCTGGAAACCGTGCAGAGACTGCTGCCCGTGCTGTGCCAGGCCC |
| | ATGGCCTGACACCTGAACAGGTGGTGGCTATCGCCTCTAATATCGGAGGAAA |
| | ACAGGCTCTGGAAACAGTGCAGCGGCTGCTGCCTGTGCTGTGTCAGGCTCAC |
| | GGCTTGACTCCAGAACAGGTGGTGGCTATTGCTTCCAACAACGGGGGGAAAC |
| | AGGCCCTGGAAACTGTGCAGCGCCTGCTGCCAGTGCTGTGCCAGGCTCACGG |
| | GCTGACCCCCGAACAGGTGGTGGCCATTGCCAGCAACGGCGGCGGCAAGCA |
| | GGCCCTGGAAACCGTGCAGAGACTGCTGCCCGTGCTGTGCCAGGCCCATGGC |
| | CTGACACCTGAACAGGTGGTGGCTATCGCCTCTAATATCGGAGGAAAACAGG |
| | CTCTGGAAACAGTGCAGCGGCTGCTGCCTGTGCTGTGTCAGGCTCACGGCTT |
| | GACTCCAGAACAGGTGGTGGCTATTGCTTCCCACGACGGGGGGAAACAGGC |
| | CCTGGAAACTGTGCAGCGCCTGCTGCCAGTGCTGTGCCAGGCTCACGGCCTC |
| | ACTCCCGAACAGGTGGTGGCCATTGCCAGCAACATCGGCGGCAAGCAGGCC |
| | CTGGAAACCGTGCAGAGACTGCTGCCCGTGCTGTGCCAGGCCCATGGCCTGA |
| | CACCTGAACAGGTGGTGGCTATCGCCTCTAACGGCGGAGGAAAACAGGCTCT |
| | GGAAACAGTGCAGCGGCTGCTGCCTGTGCTGTGTCAGGCTCACGGCTTGACT |
| | CCAGAACAGGTGGTGGCTATTGCTTCCAACAACGGGGGGAAACAGGCCCTG |
| | GAAACTGTGCAGCGCCTGCTGCCAGTGCTGTGCCAGGCTCACGGACTGACCC |
| | CCGAACAGGTGGTGGCCATTGCCAGCCACGACGGCGGCAAGCAGGCCCTGG |
| | AAACCGTGCAGAGACTGCTGCCCGTGCTGTGCCAGGCCCATGGCCTGACACC |
| | TGAACAGGTGGTGGCTATCGCCTCTCACGACGGAGGAAAACAAGCACTCGA |
| | GACAGTGCAGCGGCTGCTGCCTGTGCTGTGTCAGGCTCACGGCTTGACTCCA |
| | GAACAGGTGGTGGCTATTGCTTCCAACAACGGGGGGAAACAGGCCCTGGAA |
| | ACTGTGCAGCGCCTGCTGCCAGTGCTGTGCCAGGCTCACGGCCTGACCCCCG |
| | AACAGGTGGTGGCCATTGCCAGCAACATCGGCGGCAAGCAGGCCCTGGAAA |
| | CCGTGCAGAGACTGCTGCCCGTGCTGTGCCAGGCCCATGGCCTGACACCTGA |
| | ACAGGTGGTGGCTATCGCCTCTAACGGCGGAGGAAAACAGGCTCTGGAAAC |
| | AGTGCAGCGGCTGCTGCCTGTGCTGTGTCAGGCTCACGGCTTGACTCCACAG |
| | CAGGTCGTGGCAATTGCTAGCAACGGCGGCGGACGGCCCGCCCTGGAGAGC |
| | ATTGTGGCCCAGCTGTCTAGACCTGATCCTGCCCTGGCCGCCCTGACAAATG |
| | ATCACCTGGTGGCCCTGGCCTGTCTGGGAGGCAGACCTGCCCTGGATGCCGT |
| | GAAAAAAGGACTGCCTCACGCCCCTGCCCTGATCAAGAGAACAGGATCCGG |
| | ATCCCAGCTGGTGAAATCTGAGCTGGAGGAGAAGAAGTCTGAGCTGAGACA |
| | CAAGCTGAAGTACGTGCCTCACGAGTACATCGAGCTGATCGAGATCGCCAGA |
| | AATAGCACCCAGGATAGAATCCTGGAGATGAAGGTGATGGAGTTCTTCATGA |
| | AGGTGTACGGCTACAGAGGAAAGCACCTGGGAGGAAGCAGAAAACCTGACG |
| | GAGCCATTTATACAGTGGGCAGCCCTATCGATTATGGCGTGATCGTGGATAC |
| | AAAGGCCTACAGCGGAGGCTACAATCTGCCTATTGGACAGGCCGATGAGAT |
| | GCAGAGATACGTGGAGGAGAACCAGACCAGGAACAAGCACATCAACCCTAA |
| | CGAGTGGTGGAAGGTGTACCCTTCTAGCGTGACCGAGTTCAAGTTCCTGTTT |
| | GTGAGCGGCCACTTCAAGGGCAATTATAAGGCCCAGCTGACCAGGCTGAACC |
| | ACATCACAAATTGTAATGGCGCTGTGCTGTCTGTGGAGGAACTGCTGATTGG |
| | AGGAGAGATGATTAAGGCCGGAACACTGACACTGGAGGAGGTGAGAAGAAA |
| | GTTCAACAACGGCGAGATCAACTTCGGATCCTGA |
| 44 | MFKQASRLLSRSVAAASSKSVTTRAFSTELPSTLDSTGRATMGKPIPNPLLGLDST |
| | GGVDLRTLGYSQQQQEKIKPKVRSTVAQHHEALVGHGFTHAHIVALSQHPAAL |
| | GTVAVKYQDMIAALPEATHEAIVGVGKRGAGARALEALLTVAGELRGPPLQLD |
| | TGQLLKIAKRGGVTAVEAVHAWRNALTGAPLNLTPEQVVAIASHDGGKQALET |
| | VQRLLPVLCQAHGLTPEQVVAIASHDGGKQALETVQRLLPVLCQAHGLTPEQV |
| | VAIASNIGGKQALETVQRLLPVLCQAHGLTPEQVVAIASNGGGKQALETVQRLL |
| | PVLCQAHGLTPEQVVAIASNIGGKQALETVQRLLPVLCQAHGLTPEQVVAIASN |
| | NGGKQALETVQRLLPVLCQAHGLTPEQVVAIASNGGGKQALETVQRLLPVLCQ |
| | AHGLTPEQVVAIASNIGGKQALETVQRLLPVLCQAHGLTPEQVVAIASHDGGKQ |
| | ALETVQRLLPVLCQAHGLTPEQVVAIASNIGGKQALETVQRLLPVLCQAHGLTP |
| | EQVVAIASNGGGKQALETVQRLLPVLCQAHGLTPEQVVAIASNNGGKQALETV |
| | QRLLPVLCQAHGLTPEQVVAIASHDGGKQALETVQRLLPVLCQAHGLTPEQVV |
| | AIASHDGGKQALETVQRLLPVLCQAHGLTPEQVVAIASNNGGKQALETVQRLLP |
| | VLCQAHGLTPEQVVAIASNIGGKQALETVQRLLPVLCQAHGLTPEQVVAIASNG |
| | GGKQALETVQRLLPVLCQAHGLTPQQVVAIASNGGGRPALESIVAQLSRPDPAL |
| | AALTNDHLVALACLGGRPALDAVKKGLPHAPALIKRTGSGSQLVKSELEEKKSE |
| | LRHKLKYVPHEYIELIEIARNSTQDRILEMKVMEFFMKVYGYRGKHLGGSRKPD |
| | GAIYTVGSPIDYGVIVDTKAYSGGYNLPIGQADEMQRYVEENQTRNKHINPNEW |
| | WKVYPSSVTEFKFLFVSGHFKGNYKAQLTRLNHITNCNGAVLSVEELLIGGEMI |
| | KAGTLTLEEVRRKFNNGEINFGS |
| 45 | ATGTTTAAACAAGCTTCTCGTCTCCTCTCCCGATCTGTCGCCGCCGCATCTTC |
| | CAAATCGGTGACGACTCGTGCCTTTTCAACGGAACTTCCATCGACGCTCGATT |
| | CCGGCGCCGTGACGGTGTTCGGCATTCTGAATCTCACCGAGGACTCCTTCTTC |
| | GATGAGAGCCGGCGGCTGGACCCCGCCGGCGCTGTCACCGCGGCGATCGAA |
| | ATGCTGCGGGTCGGATCAGACGTCGTGGATGTCGGACCGGCCGCCAGCCATC |
| | CGGACGCGAGGCCTGTGTCGCCGGCCGATGAGATCAGACGCATTGCGCCGCT |
| | CTTGGACGCCCTGTCCGATCAGATGCACCGCGTTTCAATCGACAGCTTCCAA |
| | CCGGAAACCCAGCGCTATGCGCTCAAGCGCGGCGTGGGCTACCTGAACGATA |
| | TCCAAGGATTTCCTGACCCTGCGCTCTATCCCGATATTGCTGAGGCGGATTGC |
| | AGGCTGGTGGTTATGCACTCAGCGCAGCGGGATGGCATCGCCACCCGCACCG |
| | GTCACCTTCGGCCCGAAGACGCGCTCGACGAGATTGTGCGGTTCTTCGAGGC |
| | GCGGGTTTCCGCCTTGCGGCGGAGCGGGGTCGCTGCCGACCGGCTCATCCTC |
| | GATCCGGGGATGGGATTCTTCTTGAGCCCCGCACCGGAAACATCGCTGCACG |
| | TGCTGTCGAACCTTCAAAAGCTGAAGTCGGCGTTGGGGCTTCCGCTGTTGGT |
| | CTCGGTGTCGCGGAAATCCTTCTTGGGCGCTACCGTTGGCCTTCCTGTGAAGG |
| | ATCTGGGTCCAGCGAGCCTTGCGGCGGAACTTCACGCGATCGGCAATGGCGC |
| | TGACTACGTCCGCACCCACGCGCCTGGAGACCTGCGGAGCGCAATCACCATC |
| | TCGGAAACCCTCGCGAAATTTCGCAGCCGCGACGCCAGAGACCGGGGGTTG |
| | GATCATGCCTAG |
| 46 | MFKQASRLLSRSVAAASSKSVTTRAFSTELPSTLDSGAVTVFGILNLTEDSFFDES |
| | RRLDPAGAVTAAIEMLRVGSDVVDVGPAASHPDARPVSPADEIRRIAPLLDALSD |
| | QMHRVSIDSFQPETQRYALKRGVGYLNDIQGFPDPALYPDIAEADCRLVVMHSA |
| | QRDGIATRTGHLRPEDALDEIVRFFEARVSALRRSGVAADRLILDPGMGFFLSPA |
| | PETSLHVLSNLQKLKSALGLPLLVSVSRKSFLGATVGLPVKDLGPASLAAELHAI |
| | GNGADYVRTHAPGDLRSAITISETLAKFRSRDARDRGLDHA |
| 47 | MVTVFGILNLTEDSFFDESRRLDPAGAVTAAIEMLRVGSDVVDVGPAASHPDAR |
| | PVSPADEIRRIAPLLDALSDQMHRVSIDSFQPETQRYALKRGVGYLNDIQGFPDP |
| | ALYPDIAEADCRLVVMHSAQRDGIATRTGHLRPEDALDEIVRFFEARVSALRRSG |
| | VAADRLILDPGMGFFLSPAPETSLHVLSNLQKLKSALGLPLLVSVSRKSFLGATV |
| | GLPVKDLGPASLAAELHAIGNGADYVRTHAPGDLRSAITISETLAKFRSRDARDR |
| | GLDHA |
| 48 | ATGGCCGCCAAGATCCGCATCGTGATGAAATCTTTTATGAGCCAAGCTAACA |
| | AAGTTGAAGGGGTTATTCCATACGCGCAGAAGGTTGGATTGCCTGAATCACG |
| | ATCCTTGTATACCGTGCTACGATCGCCTCACATCGACAAGAAGTCGAGGGAG |
| | CAGTTCTCGATGACAGTGGCGTATATTGCCATCGGCAGCAATCTGGCCTCTCC |
| | GCTGGAGCAGGTCAATGCTGCCCTGAAAGCATTGGGCGATATCCCTGAAAGC |
| | CACATTCTTACCGTTTCTTCGTTTTACCGCACCCCACCGCTGGGACCGCAAGA |
| | TCAACCCGATTACTTGAACGCAGCCGTGGCGCTGGAAACCTCTCTTGCACCT |
| | GAAGAGCTGCTCAATCACACACAGCGTATTGAATTGCAGCAAGGTCGCGTCC |
| | GCAAAGCTGAACGCTGGGGACCACGCACGCTGGATCTCGACATCATGCTGTT |
| | TGGTAATGAAGTGATCAATACTGAACGCCTGACCGTTCCGCACTACGATATG |
| | AAGAATCGTGGATTTATGCTGTGGCCGCTGTTTGAAATCGCGCCGGAGTTGG |
| | TGTTTCCTGATGGGGAGATGTTGCGTCAAATCTTGCATACAAGAGCATTTGA |
| | CAAATTGAACAAATGGTAA |
| 49 | MAAKIRIVMKSFMSQANKVEGVIPYAQKVGLPESRSLYTVLRSPHIDKKSREQFS |
| | MTVAYIAIGSNLASPLEQVNAALKALGDIPESHILTVSSFYRTPPLGPQDQPDYLN |
| | AAVALETSLAPEELLNHTQRIELQQGRVRKAERWGPRTLDLDIMLFGNEVINTER |
| | LTVPHYDMKNRGFMLWPLFEIAPELVFPDGEMLRQILHTRAFDKLNKW |
| | AGACATATATATCTATCTAAATTTATTGATATCAATATGAATGTGGAAAATG |
| 50 | CTAAAATAACTTACATTGTGAAATGAAGGAAGTAATTTTCTTCACAAATTTTG |
| | GCACTTGCACTTACGGCTTGTGAAGCCCGCTCGATCTGAAGATTTTTCAAACG |
| | ATGCCATTCACCACGTGCCACTTTATATCAAACCTTATTTGTATCCAAGATTT |
| | CTGGTACTGGGCATTAGCGATGTCAAACAAACTCGACGTAAAGTTGAGGGAC |
| | AGAGTGTGAACTTATTTTATTCCCAGCAGAGAACGCCTCCAAGGTTTATGGG |
| | CTTTCTGGTCCATCGCAACGACACGGACTTTCTACAGGGCTGAGATTACGGG |
| | CCTAAATGGGCTCCCCCGTCTTGTGCTCCGCGCTGTTTCAAGTTGGGCCAAAA |
| | TGGAACTGGGCCGAATAGTCCGGTGCGGCTAACGGCATCCACAGCCAAGCC |
| | AACGGTGCCGTTCTCCCGCTCTCGCATTTCAAAAATCGAAAATAAAATCGGC |
| | AGGGCCGCCGACGACGCAACCACCCGTTGGGATCCAATCTACTCCTACGCAC |
| | CGAACAACTCATCGCACCCACCGATATGTGGGACCCAGCTGTAACGTGGGCC |
| | CACCGGCAAGCGGGACGTCTGCATCGGTCGCACCTACAACGAGGTGGCCAG |
| | GTCACCTACTGCTTTTTCTTGGAGCGACGCGTGCACTCGCTAACCGCACACGA |
| | AGGAAGCCTCTGGACCCACCTGTCAGTGAAACTAACGAGAGGCGGGTATGG |
| | ACTTCAACCGAGTGCAAACGAAATGGCCTCCTTGCGGTGGATATTATCAGAA |
| | AGCTCCCTAACTTATTAAATATTCTCGATATGATCCAACCACCCGACCTTTGC |
| | AGGCACGATGTGAGATGCGAGTGAATTTTGAAGACGTCAAAGGGGGTAAAA |
| | CGAAAAACTCCCCCATCCCTTCGCTTCCGCTCCTGAGAGGTCCAGGCTGTCTG |
| | GCCCTATAAAACGGGCGGAGTGCGGGGATCGCAAACCCTAAGCCGAGACAA |
| | CGCAGAGAAAGGCGTCTTCGTACTCGCCTCTCTCCGCGCCTCCGCGCTTTTCC |
| | TCCTCTCCCCTCTCTCCCTTCTCCGCCGCCGTCGCAGCATCAACCCAATCCGC |
| | CGCCAAGCTTCATCCAGGTACGGATCCGCGTCCCATCTCCCTCACCCCCCGTG |
| | TTCTTCGTGCCTGCTTCTGGGTCAGATCTGGGTGGATTCGCGGTTGTTGGATG |
| | TGGGGGGCTGTGTTTATTTGTCGGTGGATCTGGTTGTCTGGATCTGCGTTTTC |
| | TCTGTCGTAGTTAGCGGATCTGATGAAATGTTTAGTGTTCGTGTATACTGGTA |
| | TGGTGGATCTGGTCCTAGGATGCGTGGAATGGATATATGTAGGCGAATTGGA |
| | GGATTTATTTTGTGAATTTTGCTGAAATGATAGTTCTAAACACTGGATCTGAC |
| | CTCGGGATGCTGTTAAATGTGGAAATCATGGTCGATGCTGTCATGAACATGG |
| | TGTTCTTATGGTAGATCTGAGCAATGTATGTTTCAAAATTGTTTGTCACATGG |
| | AAATGCTATGGTTCTAGATGCAATAGAATGATACATGCCGAGATCCCCTCTA |
| | GTTGATATGATAGATCATGATGTTTTACAGCTATGTCATATGAATATGTTCAT |
| | TTGTTACCGATGTATTTGGATCTACTTAACATTTCCAAAGCACGCCGCGTTCT |
| | AATTCTAGATCTGGTAGTCATGTTTGTACACGTCATCCACCTAATACAAATAC |
| | ATATGTCTAGTGTTTGGTGACACTGCCCGTCAGATCTGTTTTTTCCAGATCTG |
| | TGGAACAAATACTCCATGCATGTATGGTAGTTTTGAAACGATCTTGTATCTTC |
| | CATTGTTGTAGTAACAACTAAATAAAGTACAATTGTTCAATTATTGGGAATC |
| | GTATTTTCTGTAGTGCCGATGTACAGCATATTCATAGATGTCTATTTAGGAAC |
| | TCAAATTTTAAATTGAGGACTAGTTATTTATTATGGGTCAGTCTTTTGAATTG |
| | TGTTATCTTGCTGTACTGAAATAATAATGTACCACTAAGGCGCTAACATGTAT |
| | TTTGTCCTTTCAGGCTGACTCGACGGATCCCGGGGGGCAAGAGCTCCTCCCC |
| | CCTCCCCCTCCGCCGCCGCCGGTAACCACCCCGCCCCTCTCCTCTTTCTTTCTC |
| | CGTTTTTTTTTTCGTCTCGGTCTCGATCTTTGGCCTTGGTAGTTTGGGTGGGCG |
| | AGAGCGGCTTCGTCGCCCAGATCGGTGCGCGGGAGGGGCGGGATCTCGCGG |
| | CTGGCGTCTCCGGGCGTGAGTCGGCCCGGATCCTCGCGGGGAATGGGGCTCT |
| | CGGATGTAGATCTTCTTTCTTTCTTCTTTTTGTGGTAGAATTTGAATCCCTCAG |
| | CATTGTTCATCGGTAGTTTTTCTTTTCATGATTTGTGACAAATGCAGCCTCGT |
| | GCGGAGCTTTTTTGTAGGTAGAAGATGGCAGCGAAGATCCGGATCGTCAT |
| | GAAGTCCTTCATGAGCCAGGCCAATAAGGTGGAGGGCGTCATCCCGTAT |
| | GCCCAAAAAGTCGGGCTCCCAGAGTCGCGCTCTCTCTACACAGTCTTGC |
| | GGTCGCCACATATCGATAAAAAGTCCAGAGAACAATTCTCTATGACCGG |
| | CCGCGCCACCATGGGAAAACCTATTCCTAATCCTCTGCTGGGCCTGGAT |
| | TCTACCGGAGGCGTGGACCTGAGAACACTGGGATATTCTCAGCAGCAGC |
| | AGGAGAAGATCAAGCCCAAGGTGAGATCTACAGTGGCCCAGCACCACG |
| | AAGCCCTGGTGGGACACGGATTTACACACGCCCACATTGTGGCCCTGTC |
| | TCAGCACCCTGCCGCCCTGGGAACAGTGGCCGTGAAATATCAGGATATG |
| | ATTGCCGCCCTGCCTGAGGCCACACACGAAGCCATTGTGGGAGTGGGAA |
| | AACGAGGCGCTGGAGCCAGAGCCCTGGAAGCCCTGCTGACAGTGGCCG |
| | GAGAACTGAGAGGACCTCCTCTGCAGCTGGATACAGGACAGCTGCTGAA |
| | GATTGCCAAAAGGGGCGGAGTGACCGCGGTGGAAGCCGTGCACGCCTG |
| | GAGAAATGCCCTGACAGGAGCCCCTCTGAACCTGACCCCCGAACAGGTG |
| | GTGGCCATTGCCAGCCACGACGGCGGCAAGCAGGCCCTGGAAACCGTG |
| | CAGAGACTGCTGCCCGTGCTGTGCCAGGCCCATGGCCTGACACCTGAAC |
| | AGGTGGTGGCTATCGCCTCTAACGGCGGAGGAAAACAGGCTCTGGAAA |
| | CAGTGCAGCGGCTGCTGCCTGTGCTGTGTCAGGCTCACGGCTTGACTCC |
| | AGAACAGGTGGTGGCTATTGCTTCCCACGACGGGGGGAAACAGGCCCT |
| | GGAAACTGTGCAGCGCCTGCTGCCAGTGCTGTGCCAGGCTCACGGACTG |
| | ACCCCCGAACAGGTGGTGGCCATTGCCAGCAACATCGGCGGCAAGCAG |
| | GCCCTGGAAACCGTGCAGAGACTGCTGCCCGTGCTGTGCCAGGCCCATG |
| | GCCTGACACCTGAACAGGTGGTGGCTATCGCCTCTAACGGCGGAGGAAA |
| | ACAGGCTCTGGAAACAGTGCAGCGGCTGCTGCCTGTGCTGTGTCAGGCT |
| | CACGGCTTGACTCCAGAACAGGTGGTGGCTATTGCTTCCCACGACGGGG |
| | GGAAACAGGCCCTGGAAACTGTGCAGCGCCTGCTGCCAGTGCTGTGCCA |
| | GGCTCACGGGCTGACCCCCGAACAGGTGGTGGCCATTGCCAGCAACAA |
| | CGGCGGCAAGCAGGCCCTGGAAACCGTGCAGAGACTGCTGCCCGTGCT |
| | GTGCCAGGCCCATGGCCTGACACCTGAACAGGTGGTGGCTATCGCCTCT |
| | CACGACGGAGGAAAACAGGCTCTGGAAACAGTGCAGCGGCTGCTGCCT |
| | GTGCTGTGTCAGGCTCACGGCTTGACTCCAGAACAGGTGGTGGCTATTG |
| | CTTCCCACGACGGGGGGAAACAGGCCCTGGAAACTGTGCAGCGCCTGC |
| | TGCCAGTGCTGTGCCAGGCTCACGGCCTCACTCCCGAACAGGTGGTGGC |
| | CATTGCCAGCAACATCGGCGGCAAGCAGGCCCTGGAAACCGTGCAGAG |
| | ACTGCTGCCCGTGCTGTGCCAGGCCCATGGCCTGACACCTGAACAGGTG |
| | GTGGCTATCGCCTCTAATATCGGAGGAAAACAGGCTCTGGAAACAGTGC |
| | AGCGGCTGCTGCCTGTGCTGTGTCAGGCTCACGGCTTGACTCCAGAACA |
| | GGTGGTGGCTATTGCTTCCCACGACGGGGGGAAACAGGCCCTGGAAAC |
| | TGTGCAGCGCCTGCTGCCAGTGCTGTGCCAGGCTCACGGACTGACCCCC |
| | GAACAGGTGGTGGCCATTGCCAGCAACATCGGCGGCAAGCAGGCCCTG |
| | GAAACCGTGCAGAGACTGCTGCCCGTGCTGTGCCAGGCCCATGGCCTGA |
| | CACCTGAACAGGTGGTGGCTATCGCCTCTAATATCGGAGGAAAACAAGC |
| | ACTCGAGACAGTGCAGCGGCTGCTGCCTGTGCTGTGTCAGGCTCACGGC |
| | TTGACTCCAGAACAGGTGGTGGCTATTGCTTCCAATATTGGGGGGAAAC |
| | AGGCCCTGGAAACTGTGCAGCGCCTGCTGCCAGTGCTGTGCCAGGCTCA |
| | CGGCCTGACCCCCGAACAGGTGGTGGCCATTGCCAGCAACATCGGCGG |
| | CAAGCAGGCCCTGGAAACCGTGCAGAGACTGCTGCCCGTGCTGTGCCA |
| | GGCCCATGGCCTGACACCTGAACAGGTGGTGGCTATCGCCTCTAACAAC |
| | GGAGGAAAACAGGCTCTGGAAACAGTGCAGCGGCTGCTGCCTGTGCTG |
| | TGTCAGGCTCACGGCTTGACTCCACAGCAGGTCGTGGCAATTGCTAGCA |
| | ACAACGGCGGACGGCCCGCCCTGGAGAGCATTGTGGCCCAGCTGTCTA |
| | GACCGGACCCAGCTCTCGCTGCATTGACTAACGACCATCTCGTCGCTTT |
| | GGCTTGCCTCGGTGGAAGGCCAGCTCTTGACGCTGTAAAGAAAGGTTTG |
| | CCACATGCTCCAGCTCTTATTAAAAGGACTGGTAGCGGTAGCCAACTCG |
| | TAAAGAGCGAATTGGAGGAAAAGAAAAGCGAATTGAGGCATAAACTAAA |
| | ATATGTACCACATGAATATATTGAACTCATTGAAATTGCTAGGAACTCAA |
| | CTCAAGACAGGATTCTTGAAATGAAAGTTATGGAATTTTTCATGAAAGTC |
| | TATGGTTACAGGGGGAAACATCTTGGTGGGTCAAGGAAGCCAGATGGG |
| | GCTATCTACACGGTCGGGTCACCTATTGACTACGGAGTAATTGTGGACA |
| | CCAAAGCATATTCTGGTGGGTATAACCTCCCAATCGGGCAAGCAGACGA |
| | AATGCAAAGGTATGTAGAAGAAAATCAAACTAGAAATAAACATATTAAT |
| | CCCAATGAATGGTGGAAAGTATATCCAAGCTCTGTAACTGAATTTAAATT |
| | CTTGTTCGTATCTGGTCATTTCAAAGGAAACTACAAAGCTCAACTTACTA |
| | GACTAAATCATATTACCAACTGCAACGGTGCCGTTCTCAGCGTAGAAGA |
| | ATTGCTCATCGGTGGGGAAATGATCAAAGCTGGCACGCTTACGCTTGAG |
| | GAAGTAAGGAGAAAATTCAATAACGGAGAAATTAACTTTGGCAGCTGAG |
| | AATTCATATGAAGATGAAGATGAAATATTTGGTGTGTCAAATAAAAAGCTTG |
| | TGTGCTTAAGTTTGTGTTTTTTTCTTGGCTTGTTGTGTTATGAATTTGTGGCTT |
| | TTTCTAATATTAAATGAATGTAAGATCTCATTATAATGAATAAACAAATGTTT |
| | CTATAATCCATTGTGAATGTTTTGTTGGATCTCTTCTGCAGCATATAACTACT |
| | GTATGTGCTATGGTATGGACTATGGAATATGATTAAAGATAAG |
| 51 | CTGCAGTGCAGCGTGACCCGGTCGTGCCCCTCTCTAGAGATAATGAGCATTG |
| | CATGTCTAAGTTATAAAAAATTACCACATATTTTTTTTGTCACACTTGTTTGA |
| | AGTGCAGTTTATCTATCTTTATACATATATTTAAACTTTACTCTACGAATAAT |
| | ATAATCTATAGTACTACAATAATATCAGTGTTTTAGAGAATCATATAAATGA |
| | ACAGTTAGACATGGTCTAAAGGACAATTGAGTATTTTGACAACAGGACTCTA |
| | CAGTTTTATCTTTTTAGTGTGCATGTGTTCTCCTTTTTTTTTGCAAATAGCTTC |
| | ACCTATATAATACTTCATCCATTTTATTAGTACATCCATTTAGGGTTTAGGGT |
| | TAATGGTTTTTATAGACTAATTTTTTTAGTACATCTATTTTATTCTATTTTAGC |
| | CTCTAAATTAAGAAAACTAAAACTCTATTTTAGTTTTTTTATTTAATAATTTA |
| | GATATAAAATAGAATAAAATAAAGTGACTAAAAATTAAACAAATACCCTTTA |
| | AGAAATTAAAAAAACTAAGGAAACATTTTTCTTGTTTCGAGTAGATAATGCC |
| | AGCCTGTTAAACGCCGTCGACGAGTCTAACGGACACCAACCAGCGAACCAG |
| | CAGCGTCGCGTCGGGCCAAGCGAAGCAGACGGCACGGCATCTCTGTCGCTGC |
| | CTCTGGACCCCTCTCGAGAGTTCCGCTCCACCGTTGGACTTGCTCCGCTGTCG |
| | GCATCCAGAAATTGCGTGGCGGAGCGGCAGACGTGAGCCGGCACGGCAGGC |
| | GGCCTCCTCCTCCTCTCACGGCACCGGCAGCTACGGGGGATTCCTTTCCCACC |
| | GCTCCTTCGCTTTCCCTTCCTCGCCCGCCGTAATAAATAGACACCCCCTCCAC |
| | ACCCTCTTTCCCCAACCTCGTGTTGTTCGGAGCGCACACACACACAACCAGA |
| | TCTCCCCCAAATCCACCCGTCGGCACCTCCGCTTCAAGGTACGCCGCTCGTCC |
| | TCCCCCCCCCCCCCTCTCTACCTTCTCTAGATCGGCGTTCCGGTCCATGGTTA |
| | GGGCCCGGTAGTTCTACTTCTGTTCATGTTTGTGTTAGATCCGTGTTTGTGTTA |
| | GATCCGTGCTGCTAGCGTTCGTACACGGATGCGACCTGTACGTCAGACACGT |
| | TCTGATTGCTAACTTGCCAGTGTTTCTCTTTGGGGAATCCTGGGATGGCTCTA |
| | GCCGTTCCGCAGACGGGATCGATTTCATGATTTTTTTTGTTTCGTTGCATAGG |
| | GTTTGGTTTGCCCTTTTCCTTTATTTCAATATATGCCGTGCACTTGTTTGTCGG |
| | GTCATCTTTTCATGCTTTTTTTTGTCTTGGTTGTGATGATGTGGTCTGGTTGGG |
| | CGGTCGTTCTAGATCGGAGTAGAATTAATTCTGTTTCAAACTACCTGGTGGAT |
| | TTATTAATTTTGGATCTGTATGTGTGTGCCATACATATTCATAGTTACGAATT |
| | GAAGATGATGGATGGAAATATCGATCTAGGATAGGTATACATGTTGATGCGG |
| | GTTTTACTGATGCATATACAGAGATGCTTTTTGTTCGCTTGGTTGTGATGATG |
| | TGGTGTGGTTGGGCGGTCGTTCATTCGTTCTAGATCGGAGTAGAATACTGTTT |
| | CAAACTACCTGGTGTATTTATTAATTTTGGAACTGTATGTGTGTGTCATACAT |
| | CTTCATAGTTACGAGTTTAAGATGGATGGAAATATCGATCTAGGATAGGTAT |
| | ACATGTTGATGTGGGTTTTACTGATGCATATACATGATGGCATATGCAGCATC |
| | TATTCATATGCTCTAACCTTGAGTACCTATCTATTATAATAAACAAGTATGTT |
| | TTATAATTATTTTGATCTTGATATACTTGGATGATGGCATATGCAGCAGCTAT |
| | ATGTGGATTTTTTTAGCCCTGCCTTCATACGCTATTTATTTGCTTGGTACTGTT |
| | TCTTTTGTCGATGCTCACCCTGTTGTTTGGTGTTACTTCTGCAGGTCGACTCTA |
| | GAGGATCCATGTTTAAACAAGCTTCCCGTCTTCTGTCACGCTCAGTTGCA |
| | GCTGCTTCCTCTAAGTCCGTCACCACCCGCGCTTTCTCCACCGAGCTGC |
| | CATCAACCCTGGACTCCACCGGCCGCGCCACCATGGGAAAACCGATCCC |
| | AAACCCGCTTTTGGGGCTCGACAGCACAGGAGGGGTTGATCTCAGGACC |
| | CTCGGTTACAGCCAACAGCAACAAGAAAAAATTAAGCCTAAAGTGAGGT |
| | CCACGGTAGCTCAACATCACGAGGCTTTGGTAGGGCATGGCTTCACGCA |
| | CGCTCATATCGTCGCTCTCAGCCAACATCCCGCTGCGCTTGGGACGGTC |
| | GCTGTTAAGTACCAAGACATGATCGCTGCGCTTCCGGAAGCTACGCATG |
| | AGGCGATCGTCGGTGTAGGGAAGCGGGGAGCAGGTGCGAGGGCGCTCG |
| | AGGCGCTCTTGACCGTCGCGGGTGAGCTTAGGGGTCCGCCACTCCAATT |
| | GGACACCGGACAACTTTTGAAAATCGCTAAGAGAGGAGGCGTCACAGCC |
| | GTCGAGGCGGTGCACGCCTGGAGAAATGCCCTGACAGGAGCCCCTCTG |
| | AACCTGACCCCCGAACAGGTGGTGGCCATTGCCAGCCACGACGGCGGC |
| | AAGCAGGCCCTGGAAACCGTGCAGAGACTGCTGCCCGTGCTGTGCCAG |
| | GCCCATGGCCTGACACCTGAACAGGTGGTGGCTATCGCCTCTCACGACG |
| | GAGGAAAACAGGCTCTGGAAACAGTGCAGCGGCTGCTGCCTGTGCTGT |
| | GTCAGGCTCACGGCTTGACTCCAGAACAGGTGGTGGCTATTGCTTCCAA |
| | TATTGGGGGGAAACAGGCCCTGGAAACTGTGCAGCGCCTGCTGCCAGT |
| | GCTGTGCCAGGCTCACGGACTGACCCCCGAACAGGTGGTGGCCATTGCC |
| | AGCAACGGCGGCGGCAAGCAGGCCCTGGAAACCGTGCAGAGACTGCTG |
| | CCCGTGCTGTGCCAGGCCCATGGCCTGACACCTGAACAGGTGGTGGCTA |
| | TCGCCTCTAATATCGGAGGAAAACAGGCTCTGGAAACAGTGCAGCGGCT |
| | GCTGCCTGTGCTGTGTCAGGCTCACGGCTTGACTCCAGAACAGGTGGTG |
| | GCTATTGCTTCCAACAACGGGGGGAAACAGGCCCTGGAAACTGTGCAGC |
| | GCCTGCTGCCAGTGCTGTGCCAGGCTCACGGGCTGACCCCCGAACAGGT |
| | GGTGGCCATTGCCAGCAACGGCGGCGGCAAGCAGGCCCTGGAAACCGT |
| | GCAGAGACTGCTGCCCGTGCTGTGCCAGGCCCATGGCCTGACACCTGAA |
| | CAGGTGGTGGCTATCGCCTCTAATATCGGAGGAAAACAGGCTCTGGAAA |
| | CAGTGCAGCGGCTGCTGCCTGTGCTGTGTCAGGCTCACGGCTTGACTCC |
| | AGAACAGGTGGTGGCTATTGCTTCCCACGACGGGGGGAAACAGGCCCT |
| | GGAAACTGTGCAGCGCCTGCTGCCAGTGCTGTGCCAGGCTCACGGCCTC |
| | ACTCCCGAACAGGTGGTGGCCATTGCCAGCAACATCGGCGGCAAGCAG |
| | GCCCTGGAAACCGTGCAGAGACTGCTGCCCGTGCTGTGCCAGGCCCATG |
| | GCCTGACACCTGAACAGGTGGTGGCTATCGCCTCTAACGGCGGAGGAAA |
| | ACAGGCTCTGGAAACAGTGCAGCGGCTGCTGCCTGTGCTGTGTCAGGCT |
| | CACGGCTTGACTCCAGAACAGGTGGTGGCTATTGCTTCCAACAACGGGG |
| | GGAAACAGGCCCTGGAAACTGTGCAGCGCCTGCTGCCAGTGCTGTGCCA |
| | GGCTCACGGACTGACCCCCGAACAGGTGGTGGCCATTGCCAGCCACGA |
| | CGGCGGCAAGCAGGCCCTGGAAACCGTGCAGAGACTGCTGCCCGTGCT |
| | GTGCCAGGCCCATGGCCTGACACCTGAACAGGTGGTGGCTATCGCCTCT |
| | CACGACGGAGGAAAACAAGCACTCGAGACAGTGCAGCGGCTGCTGCCT |
| | GTGCTGTGTCAGGCTCACGGCTTGACTCCAGAACAGGTGGTGGCTATTG |
| | CTTCCAACAACGGGGGGAAACAGGCCCTGGAAACTGTGCAGCGCCTGCT |
| | GCCAGTGCTGTGCCAGGCTCACGGCCTGACCCCCGAACAGGTGGTGGC |
| | CATTGCCAGCAACATCGGCGGCAAGCAGGCCCTGGAAACCGTGCAGAG |
| | ACTGCTGCCCGTGCTGTGCCAGGCCCATGGCCTGACACCTGAACAGGTG |
| | GTGGCTATCGCCTCTAACGGCGGAGGAAAACAGGCTCTGGAAACAGTGC |
| | AGCGGCTGCTGCCTGTGCTGTGTCAGGCTCACGGCTTGACTCCACAGCA |
| | GGTCGTGGCAATTGCTAGCAACGGCGGCGGACGGCCCGCCCTGGAGAG |
| | CATTGTGGCCCAGCTGTCTAGACCTGATCCTGCCCTGGCCGCCCTGACA |
| | AATGATCACCTGGTGGCCCTGGCCTGTCTGGGAGGCAGACCTGCCCTGG |
| | ATGCCGTGAAAAAAGGACTGCCTCACGCCCCTGCCCTGATCAAGAGAAC |
| | AGGATCCGGATCCCAGCTGGTGAAATCTGAGCTGGAGGAGAAGAAGTCT |
| | GAGCTGAGACACAAGCTGAAGTACGTGCCTCACGAGTACATCGAGCTGA |
| | TCGAGATCGCCAGAAATAGCACCCAGGATAGAATCCTGGAGATGAAGGT |
| | GATGGAGTTCTTCATGAAGGTGTACGGCTACAGAGGAAAGCACCTGGGA |
| | GGAAGCAGAAAACCTGACGGAGCCATTTATACAGTGGGCAGCCCTATCG |
| | ATTATGGCGTGATCGTGGATACAAAGGCCTACAGCGGAGGCTACAATCT |
| | GCCTATTGGACAGGCCGATGAGATGCAGAGATACGTGGAGGAGAACCA |
| | GACCAGGAACAAGCACATCAACCCTAACGAGTGGTGGAAGGTGTACCCT |
| | TCTAGCGTGACCGAGTTCAAGTTCCTGTTTGTGAGCGGCCACTTCAAGG |
| | GCAATTATAAGGCCCAGCTGACCAGGCTGAACCACATCACAAATTGTAA |
| | TGGCGCTGTGCTGTCTGTGGAGGAACTGCTGATTGGAGGAGAGATGATT |
| | AAGGCCGGAACACTGACACTGGAGGAGGTGAGAAGAAAGTTCAACAAC |
| | GGCGAGATCAACTTCGGATCCTGAGAATTCGGTACCGAGTTTGAATCAAAT |
| | CTTCACTTGTTTAATGAGATATGCGAGACGCCTATGATCGCATGATATTTGCT |
| | TTCAATTCTGTTGTGCACGTTGTAAAAAACCTGAGCATGTGTAGCTCAGATCC |
| | TTACCGCCGGTTTCGGTTCATTCTAATGAATATATCACCCGTTACTATCGTAT |
| | TTTTATGAATAATATTCTCCGTTCAATTTACTGATTGTACCCTACTACTTATAT |
| | GTACAATATTAAAATGAAAACAATATATTGTGCTGAATAGGTTTATAGCGAC |
| | ATCTATGATAGAGCGCCACAATAACAAACAATTGCGTTTTATTATTACAAAT |
| | CCAATTTTAAAAAAAGCGGCAGAACCGGTCAAACCTAAAAGACTGATTACAT |
| | AAATCTTATTCAAATTTCAAAAGTGCCCCAGGGGCTAGTATCTACGACACAC |
| | CGAGCGGCGAATTCAGTACATTAAAAACGTCCGCAATGTGTTATTAAGTTGT |
| | CTAAGCGTCAATTTGTTTACACCACAATATATCCTGCCACCAGCCAGCCAAC |
| | AGCTCCCCGACCGGCAGCTCGGCACAAAATCACCACTCGATACAGGCAGCCC |
| | ATCAGTCCGGGACGGCGTCAGCGGGA |
| 52 | TAGCTAGCATACTCGAGGTCATTCATATGCTTGAGAAGAGAGTCGGGATAGT |
| | CCAAAATAAAACAAAGGTAAGATTACCTGGTCAAAAGTGAAAACATCAGTT |
| | AAAAGGTGGTATAAGTAAAATATCGGTAATAAAAGGTGGCCCAAAGTGAAA |
| | TTTACTCTTTTCTACTATTATAAAAATTGAGGATGTTTTGTCGGTACTTTGATA |
| | CGTCATTTTTGTATGAATTGGTTTTTAAGTTTATTCGCGATTTGGAAATGCAT |
| | ATCTGTATTTGAGTCGGTTTTTAAGTTCGTTGCTTTTGTAAATACAGAGGGAT |
| | TTGTATAAGAAATATCTTTAAAAAACCCATATGCTAATTTGACATAATTTTTG |
| | AGAAAAATATATATTCAGGCGAATTCCACAATGAACAATAATAAGATTAAA |
| | ATAGCTTGCCCCCGTTGCAGCGATGGGTATTTTTTCTAGTAAAATAAAAGAT |
| | AAACTTAGACTCAAAACATTTACAAAAACAACCCCTAAAGTCCTAAAGCCCA |
| | AAGTGCTATGCACGATCCATAGCAAGCCCAGCCCAACCCAACCCAACCCAAC |
| | CCACCCCAGTGCAGCCAACTGGCAAATAGTCTCCACCCCCGGCACTATCACC |
| | GTGAGTTGTCCGCACCACCGCACGTCTCGCAGCCAAAAAAAAAAAAAGAAA |
| | GAAAAAAAAGAAAAAGAAAAACAGCAGGTGGGTCCGGGTCGTGGGGGCCG |
| | GAAAAGCGAGGAGGATCGCGAGCAGCGACGAGGCCCGGCCCTCCCTCCGCT |
| | TCCAAAGAAACGCCCCCCATCGCCACTATATACATACCCCCCCCTCTCCTCCC |
| | ATCCCCCCAACCCTACCACCACCACCACCACCACCTCCTCCCCCCTCGCTGCC |
| | GGACGACGAGCTCCTCCCCCCTCCCCCTCCGCCGCCGCCGGTAACCACCCCG |
| | CCCCTCTCCTCTTTCTTTCTCCGTTTTTTTTTTCGTCTCGGTCTCGATCTTTGGC |
| | CTTGGTAGTTTGGGTGGGCGAGAGCGGCTTCGTCGCCCAGATCGGTGCGCGG |
| | GAGGGGCGGGATCTCGCGGCTGGCGTCTCCGGGCGTGAGTCGGCCCGGATCC |
| | TCGCGGGGAATGGGGCTCTCGGATGTAGATCTTCTTTCTTTCTTCTTTTTGTG |
| | GTAGAATTTGAATCCCTCAGCATTGTTCATCGGTAGTTTTTCTTTTCATGATTT |
| | GTGACAAATGCAGCCTCGTGCGGAGCTTTTTTGTAGGTAGAAGATGTTTAAA |
| | CAAGCTTCTCGTCTCCTCTCCCGATCTGTCGCCGCCGCATCTTCCAAATC |
| | GGTGACGACTCGTGCCTTTTCAACGGAACTTCCATCGACGCTCGATTCC |
| | GGCGCCGTGACGGTGTTCGGCATTCTGAATCTCACCGAGGACTCCTTCT |
| | TCGATGAGAGCCGGCGGCTGGACCCCGCCGGCGCTGTCACCGCGGCGA |
| | TCGAAATGCTGCGGGTCGGATCAGACGTCGTGGATGTCGGACCGGCCG |
| | CCAGCCATCCGGACGCGAGGCCTGTGTCGCCGGCCGATGAGATCAGAC |
| | GCATTGCGCCGCTCTTGGACGCCCTGTCCGATCAGATGCACCGCGTTTC |
| | AATCGACAGCTTCCAACCGGAAACCCAGCGCTATGCGCTCAAGCGCGGC |
| | GTGGGCTACCTGAACGATATCCAAGGATTTCCTGACCCTGCGCTCTATC |
| | CCGATATTGCTGAGGCGGATTGCAGGCTGGTGGTTATGCACTCAGCGCA |
| | GCGGGATGGCATCGCCACCCGCACCGGTCACCTTCGGCCCGAAGACGC |
| | GCTCGACGAGATTGTGCGGTTCTTCGAGGCGCGGGTTTCCGCCTTGCGG |
| | CGGAGCGGGGTCGCTGCCGACCGGCTCATCCTCGATCCGGGGATGGGA |
| | TTCTTCTTGAGCCCCGCACCGGAAACATCGCTGCACGTGCTGTCGAACC |
| | TTCAAAAGCTGAAGTCGGCGTTGGGGCTTCCGCTGTTGGTCTCGGTGTC |
| | GCGGAAATCCTTCTTGGGCGCTACCGTTGGCCTTCCTGTGAAGGATCTG |
| | GGTCCAGCGAGCCTTGCGGCGGAACTTCACGCGATCGGCAATGGCGCT |
| | GACTACGTCCGCACCCACGCGCCTGGAGACCTGCGGAGCGCAATCACCA |
| | TCTCGGAAACCCTCGCGAAATTTCGCAGCCGCGACGCCAGAGACCGGG |
| | GGTTGGATCATGCCTAGGATCGTTCAAACATTTGGCAATAAAGTTTCTTAA |
| | GATTGAATCCTGTTGCCGGTCTTGCGATGATTATCATATAATTTCTGTTGAAT |
| | TACGTTAAGCATGTAATAATTAACATGTAATGCATGACGTTATTTATGAGAT |
| | GGGTTTTTATGATTAGAGTCCCGCAATTATACATTTAATACGCGATAGAAAA |
| | CAAAATATAGCGCGCAAACTAGGATAAATTATCGCGCGCGGTGTCATCTATG |
| | TTACTAGATC |
| 53 | ATGGTGGAGCACGACACTCTCGTCTACTCCAAGAATATCAAAGATACAGTCT |
| | CAGAAGACCAAAGGGCTATTGAGACTTTTCAACAAAGGGTAATATCGGGAA |
| | ACCTCCTCGGATTCCATTGCCCAGCTATCTGTCACTTCATCAAAAGGACAGTA |
| | GAAAAGGAAGGTGGCACCTACAAATGCCATCATTGCGATAAAGGAAAGGCT |
| | ATCGTTCAAGATGCCTCTGCCGACAGTGGTCCCAAAGATGGACCCCCACCCA |
| | CGAGGAGCATCGTGGAAAAAGAAGACGTTCCAACCACGTCTTCAAAGCAAG |
| | TGGATTGATGTGATAACATGGTGGAGCACGACACTCTCGTCTACTCCAAGAA |
| | TATCAAAGATACAGTCTCAGAAGACCAAAGGGCTATTGAGACTTTTCAACAA |
| | AGGGTAATATCGGGAAACCTCCTCGGATTCCATTGCCCAGCTATCTGTCACTT |
| | CATCAAAAGGACAGTAGAAAAGGAAGGTGGCACCTACAAATGCCATCATTG |
| | CGATAAAGGAAAGGCTATCGTTCAAGATGCCTCTGCCGACAGTGGTCCCAAA |
| | GATGGACCCCCACCCACGAGGAGCATCGTGGAAAAAGAAGACGTTCCAACC |
| | ACGTCTTCAAAGCAAGTGGATTGATGTGATATCTCCACTGACGTAAGGGATG |
| | ACGCACAATCCCACTATCCTTCGCAAGACCTTCCTCTATATAAGGAAGTTCAT |
| | TTCATTTGGAGAGGACACGCTGAAATCACCAGTCTCTCTCTACAAATCTATCT |
| | CTCTCGAGCTTTCGCAGATCCCGGGGGGCAATGAGATATGGCCGCCAAGATC |
| | CGCATCGTGATGAAATCTTTTATGAGCCAAGCTAACAAAGTTGAAGGGGTTA |
| | TTCCATACGCGCAGAAGGTTGGATTGCCTGAATCACGATCCTTGTATACCGT |
| | GCTACGATCGCCTCACATCGACAAGAAGTCGAGGGAGCAGTTCTCGATGACA |
| | GTGGCGTATATTGCCATCGGCAGCAATCTGGCCTCTCCGCTGGAGCAGGTCA |
| | ATGCTGCCCTGAAAGCATTGGGCGATATCCCTGAAAGCCACATTCTTACCGT |
| | TTCTTCGTTTTACCGCACCCCACCGCTGGGACCGCAAGATCAACCCGATTACT |
| | TGAACGCAGCCGTGGCGCTGGAAACCTCTCTTGCACCTGAAGAGCTGCTCAA |
| | TCACACACAGCGTATTGAATTGCAGCAAGGTCGCGTCCGCAAAGCTGAACGC |
| | TGGGGACCACGCACGCTGGATCTCGACATCATGCTGTTTGGTAATGAAGTGA |
| | TCAATACTGAACGCCTGACCGTTCCGCACTACGATATGAAGAATCGTGGATT |
| | TATGCTGTGGCCGCTGTTTGAAATCGCGCCGGAGTTGGTGTTTCCTGATGGGG |
| | AGATGTTGCGTCAAATCTTGCATACAAGAGCATTTGACAAATTGAACAAATG |
| | GTAAAGCAGTAGATGCCGACCGGATCTGTCGATCGACAAGCTCGAGTTTCTC |
| | CATAATAATGTGTGAGTAGTTCCCAGATAAGGGAATTAGGGTTCCTATAGGG |
| | TTTCGCTCATGTGTTGAGCATATAAGAAACCCTTAGTATGTATTTGTATTTGT |
| | AAAATACTTCTATCAATAAAATTTCTAATTCCTAAAACCAAAATCCAGTACT |
| | AAAATCCAGATCCCCCGAATTA |
| 54 | GCTACTGGTGTTGGGCAACATCAA |
| 55 | ATATGTATGCGCCCTATTCGCCTTA |
| 56 | TCGGTAATCCGAAGCCCACC |
| 57 | CCATAGTACATGCCGATTGAAGGATG |
| 58 | GGGCCCAAATTCAATTGTATATGAGCTCATATACAAGACCTCACTAGTAAGG |
| | AAGGCACTTGCTGCCGGAGTTCAACAGGCAAATATAAGAAAAGAAGTCCTG |
| | TTCACTTCATCATCTGTGGGTTGTACTGCTTGAAGGTTCTTCTGAGGGGTAGA |
| | ATTTGAATTCCTTCTTTGCTTGTGAGATAACCATTTCCAGAAACTCATATATA |
| | GAGAGCGGGTATCGGTGAAAATGGATCTTACCAGGAGTGGCATTGAATAGG |
| | CAGGCTCTGGGATGTAATCTCACTCAAGAGGTCATTTGTTGGCCCCGCCTTCA |
| | CTAGACTAGAGTTTTAGGATAGGTTGGGGAACCTATACGTCAAGCCCCTACG |
| | AAGATTGAGAAAAATCGATGCACATAAGCCATCCGAAACCAGTATTGGAAA |
| | GTGTTCAGTTTCGTTTTCCATTCTGAAATGTTCATAGTAGTATAGTATGTTTTC |
| | CGTTGGGTCGACGCCATGTGATCGCTACTAAAGATAGAGTTTCCTTGGAAAA |
| | ACCGAGGCCAGTTGAGATCAGTCTCCCTTTCTAGGAGCAGAGCTTAAAAAGA |
| | TGGGAAATTCCAATGAATTTCGATCACAATCATGTGGTAATAATGGGTTTGA |
| | ATCAGAGAGACTCGATCTGGAAACTCCTCAATGATTATAACGTGAACTCGTT |
| | GAAGAGAAGGAGACAAGCAGAAATAGACGCTTTTTTTGAACCATTTGAGAG |
| | GGCGCAGCGTATCCGTTTCAATAACTGGCAGAACGGAATAGAGTTGTTAGAT |
| | GGGGCTGAATGGAGGAACGGCGATATAGTTATCCCTGGAGGCGGCGGACCA |
| | GTAATTTCAAGCCCCTTGGATCAATTTTTCATTGATCCATTATTTGGTCTTGAT |
| | ATGGGTAACTTTTATTTATCATTCACAAATGAATCCTTGTCTATGGCGGTAAC |
| | TGTCGTTTTGGTGCCATCTTTATTTGGAGTTGTTACGAAAAAGGGCGGGGGA |
| | AAGTCAGTGCCAAATGCATGGCAATCCTTGGTAGAGCTTATTTATGATTTCGT |
| | GCTGAACCTGGTAAACGAACAAATAGGTGGAAATGTTAAACAAAAGTTTTTC |
| | CCTCGCATCTCGGTCACTTTTACTTTTTCGTTATTTCGTAATCCCCAGGGTATG |
| | ATACCCTTTAGCTTCACAGTGACAAGTCATTTTCTCATTACTTTGGCTCTTTCA |
| | TTTTCCATTTTTATAGGCATTACGATCGTTGGATTTCAAAGACATGGGCTTCA |
| | TTTTTTTAGCTTCTTATTACCAGCGGGAGTCCCACTGCCATTAGCACCTTTTTT |
| | AGTACTCCTTGAGCTAATCTCTCATTGTTTTCGTGCATTAAGCTCAGGAATAC |
| | GTTTATTTGCTAATATGATGGCCGGTCATAGTTCAGTAAAGATTTTAAGTGGG |
| | TTCGCTTGGACTATGCTATTTCTGAATAATATTTTCTATTTCATAGGAGATCTT |
| | GGTCCCTTATTTATTGTATTGGCTCTTACTGGATTGGAATTAGGTGTAGCTAT |
| | ATTACAAGCTCATGTTTCTACGATCTCAATTTGTATTTACTTGAATGATGCTA |
| | TAAATCTCCATCAAAATGAGTAATTTCATAATTGAATAAAAACGAGGAGCCG |
| | AAGATTTTAGGGGGCGGGACAAACGCGGAAGTGTATTGCGTTACAAAAAAT |
| | GACAACTAGCATTTGTTTTTTCATTTCATGTTCGAATTCGTTTTTCGTTGGAAA |
| | AACCAACGCCGACCCCAAACAAGTCTCTCCAATATAAGGAGAGCGGAGCTT |
| | AAAAATATTATTTTATTGTGCTATGGCAAATCTGGTCCGATGGCTCTTCTCCA |
| | CTACCCGAGGGACTAACGGTCTTCCATATTTCATCTTCGGTGTCGTTGTAGGA |
| | GGCGCCCTGTTGTTTGCTTTGCTAAAGTATCAGGCCCCTCTGTACGACCCGGC |
| | TTTAATGGAAAAAATCATAGATCATAATATAAAAGCCGGGCACCCTATAGAG |
| | GTTGACTATTCGTGGTGGGGCACCTCTATTCGTGTAGTCTTTCCTAAGTAAGA |
| | AAGACAGGACAGTGGTGGTTTGCTCATACTTTCATTACAAAACCATACTATG |
| | GAATTAGGGATAACAGGGTAATAATCACAAGTGAGAACCACAGGTAGCAAT |
| | AGGTATTACAGAAATTTCCTCGAGTCTGCTTGAAAGCCTGCAGAGTCCAATT |
| | TTGAGTATTTTCAGTTAGAATCTAGAGTCAGCCTATTCAGTTCTTAGCCCTTA |
| | AGGGTAAGGCAGGGGGTAATATGGATAGTCTCTGTCCCTGTATTCACATTCC |
| | ACCTTCAACAAAGTGTTGATTTCCCGTAAAGCTAACTGTAGTCCTTTAAGTAA |
| | GTAGATATCTTAGGCAAGTTAGCAATCTCGTTATATTACCAAGGCCTTCCCTT |
| | CTATTGTAGAAAGAGTTCTCAGCCATCTAATTGCAGTGCCAGTTGCCAGCTAT |
| | CCAGTTTCATTTGAAGTTGCTGGGGGTCCAAACGAGCTAGTTGCTTTTATTCG |
| | TCCTATAAGTCCTTCCACAAGCGAGTCAATAGGGTGCTGGCTAGTTGTAGTT |
| | GTTGGCGTGCCTTTCCTTTCATCTTGAATATTAATAAATATTTGGATAAATTA |
| | CTTTAGAATAAGAAGTTCATGTTTTAATACGACTCACTATAGTAAGTAATAC |
| | GAATCCATACTAGGAAAATGAAAATGTGAGTCCTAGGCACTGGAATTGGTTC |
| | TCTTCTCCCTAATCCCTATAAGCCAGAAAGGGTAATAGGCTTCAGTGTAAGC |
| | ATTTCCTTCAAGCAAGTCATCTCAAGTTTTAAATTCTAGAGAATAGCTCCGAT |
| | CAACCCATTTTAGTTTGGTTCTGCAATTCATTCGCATAAATGAAAAAAAAAG |
| | CGAGATGTGCACGAAAGAAGATCATAGTTCAGCTTTAAAATGGTGGTGTCCC |
| | TGTGTTAGTAAGTGGTTGAAATAGCTCATGGGAGTGTCTGCCCCATTCGATA |
| | ATGGCATTTATGATCTAGTGGAGTGAGTGATTGTGTGGTGTTCAGTCTAAGG |
| | CTTTTTGAAAAGCGGATTTCTCCCTTCTCTCATCCATCGTCTTTGTTAAAGTGA |
| | ATTTCTCTGACATTCCATGTTTCCGAAAcGGATCCTATAAAGACCGGCCGTA |
| | AGAACCATCAACGACATCATGTCCTTCCCGCACGAGGCCGAGTGGGGGC |
| | GGCGGCGGTCAGGTGCTCGGCGGTGTCCCCAGTCACCCCACCATCCCCA |
| | GCGCCACCAGCCACTCCACTCCGACCATGGGGGCCAGCCGAGCCCCGT |
| | AAGGGCGCGGACATCCTCGTGGAGGCGCTGGAGCGATGCGGCGTCAGC |
| | GACGTGTTCGCCTATCCAGGCGGCGCGTCCATGGAGATCCATCAAGCGC |
| | TGACTCGTTCCCCAGTCATCACCAACCATCTCTTCCGTCATGAGCAAGG |
| | CGAGGCGTTCGCGGCGTCCGGGTATGCGCGTGCGTCCGGCCGTGTCGG |
| | GGTCTGCGTCGCCACCTCCGGCCCCGGGGCAACCAACCTCGTGTCCGCG |
| | CTCGCCGACGCGCTGCTCGACTCCGTCCCAATGGTCGCCATCACTGGCC |
| | AAGTCCCCCGTCGTATGATCGGCACCGACGCCTTCCAAGAGACTCCCAT |
| | AGTCGAGGTCACCCGTTCCATCACCAAGCATAATTATCTTGTCCTTGATG |
| | TGGAGGACATCCCCCGTGTCATACAAGAAGCCTTCTTCCTCGCGTCCTC |
| | GGGCCGTCCTGGCCCAGTGCTGGTCGACATCCCCAAGGACATCCAACAA |
| | CAAATGGCCGTGCCAGTCTGGGACACCTCGATGAATCTACCAGGGTATA |
| | TCGCACGTCTGCCCAAGCCACCCGCGACAGAATTGCTTGAGCAAGTCTT |
| | GCGTCTGGTTGGCGAGTCACGACGTCCAATTCTCTATGTCGGTGGTGGC |
| | TGCTCTGCATCTGGTGACGAATTGCGTTGGTTTGTTGAGCTGACTGGTA |
| | TCCCAGTTACAACCACTCTGATGGGCCTCGGCAATTTCCCCAGTGACGA |
| | CCCATTGTCCCTGCGTATGCTTGGGATGCATGGCACTGTGTATGCAAAT |
| | TATGCCGTGGATAAGGCTGACCTGTTGCTTGCGTTTGGTGTGCGATTTG |
| | ATGATCGTGTGACAGGGAAAATTGAGGCTTTTGCAAGCAGGGCCAAGAT |
| | TGTGCATATTGACATTGATCCAGCAGAGATTGGAAAGAACAAGCAACCA |
| | CATGTGTCAATTTGCGCAGATGTTAAACTTGCTTTACAAGGCTTGAATGC |
| | TCTGCTACAACAAAGCACAACAAAGACAAGTTCTGATTTTAGTGCATGG |
| | CATAATGAGTTGGACCAACAAAAGAGGGAGTTTCCTCTGGGGTATAAAA |
| | CTTTTGGTGAAGAGATCCCACCACAATATGCCATTCAAGTGCTGGATGA |
| | GCTGACTAAAGGTGAGGCAATCATCGCTACTGGTGTTGGGCAACATCAA |
| | ATGTGGGCGGCACAATATTATACCTATAAGCGACCACGACAATGGCTGT |
| | CTTCGGCTGGTCTGGGCGCAATGGGATTTGGGCTGCCTGCTGCAGCTGG |
| | TGCTTCTGTGGCTAACCCAGGTGTCACAGTTGTTGATATTGATGGGGAT |
| | GGTAGCTTCCTCATGAACATTCAAGAGCTGGCATTGATCCGTATTGAGA |
| | ACCTCCCTGTGAAGGTGATGGTGTTGAACAACCAACATTTGGGTATGGT |
| | GGTGCAATTGGAGGATAGGTTTTATAAGGCGAATAGGGCGCATACATAT |
| | TTGGGCAACCCAGAATGTGAGAGCGAGATATATCCAGATTTTGTGACTA |
| | TTGCTAAGGGGTTCAATATTCCTGCAGTCCGTGTAACAAAGAAGAGTGA |
| | AGTCCGTGCCGCCATCAAGAAGATGCTTGAGACTCCAGGGCCATATTTG |
| | TTGGATATCATCGTCCCACATCAAGAGCATGTGCTGCCTATGATCCCAAT |
| | TGGGGGCGCATTCAAGGACATGATCCTGGATGGTGATGGCAGGACTGT |
| | GTATTAATGAGGTACCAAGCGATCGCAAACCTAGGAAAAGATCTAGACGAG |
| | GTGTAGCGCAGTCTGGTCAGCGCATCTGTTTTGGGTACAGAGGGCCATAGGT |
| | TCGAATCCTGTCACCTTGAGTCTGGCCCCAAATTCTAATTTCTACTGTTGTAG |
| | ATTTCAATTATGAAATTACTCATGTCCCTTTCGTCCAGTGGTTAGGACATCGT |
| | CTTTTCATGTCGAAGACACGGGTTCGATTCCCGTAAGGGATAGTCTGGCCCC |
| | AAATTCTAATTTCTACTGTTGTAGATGTCACATTTCTACCGGTGCACATTCCA |
| | GCTTATTTGATACCCACTTCAAGTTTCTATCAAACCATGTCTTTTTCTTCGAAC |
| | GTCAATCTCGTAGGAAAGATCTAAAAAGCTTAAGCGGCCGCAAAAACCCCTT |
| | GGGGCCTCTAAACGGGTCTTGAGGGGTTTTTTGTAGATAGACCTTTTTATTTT |
| | TCGTCATTCGATCACGAAAAGGCCGGCCAAACCTCGAGGTAATTAAAGCGGC |
| | CGAAATTAGCTAGCAAATAAGCATGCAATCACAAGTGAGAACCACAGGTAG |
| | CAATAGGTATTACAGTAGGGATAACAGGGTAATGAATTCGAAACCATCTTCT |
| | CACTCTGACCCCCACATATCAGATCCCAGATGCATAGGAAAAGCGGTATCAA |
| | GAATAGTAGTATAAAGAAAGATAGTACAGTACTCAAGTAAATGAATTCGCCT |
| | AAGGATCGATGGAAAGATCAAGGTCCCCGTGAAAAAGTAGATACTAGATCG |
| | ATATGATACTCTCATCTCTGGAGTAACTTCTTCCATTATGCTGATCTCTAGGT |
| | CCGTTCCATCATCATCGTAATAGTATGGTCCCAGGTGTCCGAGCTATAGATCA |
| | AGATCATATCCAGTCACATTTCTACCGGTGCACTTCTCATGAAATAATTCCCT |
| | TTCCAAGGAAAGGAAAACAAGAACTCGAATACTCGTAATAGCGATCCCGAT |
| | CCACCTACTTTTTTCTATTCTTTGATTCGAAACGTGCTAAAGCACAAGCCATT |
| | TTTATGCATGGGGCATAAGAGTGGACAATCTATGTTATCGAAGGAAGTAAAT |
| | AACAACACTTCAGCGTTTAGGTCTACCTTCAGTAAACCAATAGTTTTGCAGC |
| | ATTGGAATTTGAGTTGGCCAGGTAAGGTCCTCTAAAAAGAAAAGAAGAAAC |
| | TACTTAGAATAGATAAATGCCATTGGTTTTCTCGTACTATACGATCTTTTTTT |
| | GTTTTGTTTTTTGGCCATGATTGTGCTGCTCCTGTGAAGGCTAGTGGGAAAGC |
| | TCACCGTTCGTTGTGATGAGTGGGGGCCTTGTATCTGTATTCGGATCAGCTCC |
| | TTAACAGAGTTTCCTGCTTGAACCCTGGCTGGGAGCTGGGAGAGGTGTCCCA |
| | CTACAGGTGCAAATAAACCATTTGACCTTACAGGGGAAAGGAAACAAACCA |
| | CTCAATAATCGGTAGAAATTCCTCCTACTGAACAGCTTTCCTTTTCTCGCCTT |
| | AACTACTACTTCAAAGCAAGGCGGAATATCACGGGATAGGAATGAAAGAAC |
| | TTCTTACTCAACTTTCTAGCTATATAAAAATAGTTAGCAATATGAAACGAGTA |
| | ACTTAAGCCCTAGTAAAAGGCTACTCTTTGAATCCCCTCTTTAAGGCATATAA |
| | AATTAGTACTCTTCCTGAGCTAGCTTAAGCATATCTTGAGCGAGTGAGTTGTA |
| | TTTCCCTCCATCAAGTTCTAAGCGATCAAATAAGGTCCTTGCTCTCGAGCCAA |
| | TGCCAATACCAATAGAGAGGGTCTAAACGAAGGATTCAAAGGCGCGCC |
| 59 | ATGTTTAAACAAGCTTCTCGTCTCCTCTCCCGATCTGTCGCCGCCGCATCTTC |
| | CAAATCGGTGACGACTCGTGCCTTTTCAACGGAACTTCCATCGACGCTCGATT |
| | CCGGCGCCGCCGCTCCCTCTCCGGCCACCGGCGGCGTCGCGCCGGTTCCGCC |
| | AAGCCCCAGGGGCTCGATTATAAAGCGTCATACACTATCAGTTTTTGTTGGA |
| | GATGAAAGTGGGATGATTAATCGAATTGCTGGAGTTTTTGCTAGAAGAGGAT |
| | ACAACATTGAGTCATTGGCAGTTGGGCTGAACAAGGACAAGGCATTATTTAC |
| | AATAGTAGTATCTGGAACTGAGAAAATACTGAAACAGGTTGTAGAGCAACT |
| | AAACAAACTTGTTAATGTTATACAGGTTGACGATCTGTCGAAGGAACCACAA |
| | GTTGAAAGAGAGCTAATGCTTATAAAACTAAATGTTGAGCCAGATAAGCGAC |
| | CGGAGGTGATGGGTTTGGTTGACATCTTCAGAGCAAAGGTGGTTGACCTTTC |
| | TGACCACACACTAACTATTGAGGTAACTGGAGATCCTGGAAAAATTGTTGCT |
| | GTACAGAGGAACCTAAGCAAATTTGGAATCAAAGAAATTGCCAGAACTGGC |
| | AAGATAGCTTTGCGGCGTGAGAAAATGGGAGAAAGTGCTCCTTTTTGGCGGT |
| | TCTCTGCAGCTTCCTATCCTGATCTTGAAGTGGCAATGCCATCAAAATCTCAT |
| | GTCAACACTGCGATGAAAACAGCTAACCAGAATTCTGAAGAATCTTCACAAG |
| | GTGATGTCTATCCAGTGGAGTCATATGAAAACTTCACAACAAATCAAATTCT |
| | TGATGCTCATTGGGGTGTCATGGCTGACGGTGATCCAACAGGGCTTTGTTCA |
| | CATTCTTTGTCCATTTTGGTGAATGATTTCCCTGGAGTTCTCAATGTTGTAAC |
| | AGGTGTTTTCTCCCGAAGAGGCTACAATATTCAGAGTCTGGCTGTTGGCCCA |
| | GCTGAAAAAGAAGGCACTTCTCGGATCACTACTGTTGTCCCTGGAACTGATG |
| | AGTCTATTGCCAAGCTAGTACACCAACTGTATAAGCTCATTGATGTTTATGAG |
| | GTCCAGGATCTTACTCATTTACCATTTGCTGCTAGAGAGTTAATGATCATAAA |
| | GATTGCTGTAAACACCACAGCCCGCAGGGCTATCCTAGATATTGCTGATATT |
| | TTCCGGGCCAAAACTGTGGATGTATCAGATCACACCGTAACTCTTCAGCTTA |
| | CTGGAGACCTTGATAAAATGGTCGCATTACAAAGGATGCTCGAGCCCTATGG |
| | CATTTGTGAGGTTGCACGAACTGGCAGGGTTGCGTTGCGCCGTGAGTCAGGA |
| | GTCGATTCCAAATACCTCCGTGGGTTTTCCCTTCCTCTGTAG |
| 60 | MNELTLHPNVYATGASKGLIHVLERVWVREHTPGDGTIFVISGFGNYNGGVRFF |
| | ESFREHKRRGGELVAVFAGSTSARLTSKQVVHEMLDCGADVHIVNRKRLLHAK |
| | CYGTSKMSGDSLIVTSGNFTGPGMSQNVEMSVLLDADTTAEMGFSWGTLLENL |
| | LDQRWNYHRPTLGDPANPAWSLLYDEQAADIRLDETEE |
| 61 | DQRWNYHRPTLGDPANPAWSLLYDEQAADIRLDETEE |
| 62 | GCAGCCTCATCGCCAACA |
| 63 | ATGGCAGCGAAGATCCGGATCGTCATGAAGTCCTTCATGAGCCAGGCCAATA |
| | AGGTGGAGGGCGTCATCCCGTATGCCCAAAAAGTCGGGCTCCCAGAGTCGCG |
| | CTCTCTCTACACAGTCTTGCGGTCGCCACATATCGATAAAAAGTCCAGAGAA |
| | CAATTCTCTATGACCGGCCGCGCCACCATGGGAAAACCTATTCCTAATCCTCT |
| | GCTGGGCCTGGATTCTACCGGAGGCGTGGACCTGAGAACACTGGGATATTCT |
| | CAGCAGCAGCAGGAGAAGATCAAGCCCAAGGTGAGATCTACAGTGGCCCAG |
| | CACCACGAAGCCCTGGTGGGACACGGATTTACACACGCCCACATTGTGGCCC |
| | TGTCTCAGCACCCTGCCGCCCTGGGAACAGTGGCCGTGAAATATCAGGATAT |
| | GATTGCCGCCCTGCCTGAGGCCACACACGAAGCCATTGTGGGAGTGGGAAA |
| | ACGAGGCGCTGGAGCCAGAGCCCTGGAAGCCCTGCTGACAGTGGCCGGAGA |
| | ACTGAGAGGACCTCCTCTGCAGCTGGATACAGGACAGCTGCTGAAGATTGCC |
| | AAAAGGGGCGGAGTGACCGCGGTGGAAGCCGTGCACGCCTGGAGAAATGCC |
| | CTGACAGGAGCCCCTCTGAACCTGACCCCCGAACAGGTGGTGGCCATTGCCA |
| | GCAACAACGGCGGCAAGCAGGCCCTGGAAACCGTGCAGAGACTGCTGCCCG |
| | TGCTGTGCCAGGCCCATGGCCTGACACCTGAACAGGTGGTGGCTATCGCCTC |
| | TCACGACGGAGGAAAACAGGCTCTGGAAACAGTGCAGCGGCTGCTGCCTGT |
| | GCTGTGTCAGGCTCACGGCTTGACTCCAGAACAGGTGGTGGCTATTGCTTCC |
| | AATATTGGGGGGAAACAGGCCCTGGAAACTGTGCAGCGCCTGCTGCCAGTGC |
| | TGTGCCAGGCTCACGGACTGACCCCCGAACAGGTGGTGGCCATTGCCAGCAA |
| | CAACGGCGGCAAGCAGGCCCTGGAAACCGTGCAGAGACTGCTGCCCGTGCT |
| | GTGCCAGGCCCATGGCCTGACACCTGAACAGGTGGTGGCTATCGCCTCTCAC |
| | GACGGAGGAAAACAGGCTCTGGAAACAGTGCAGCGGCTGCTGCCTGTGCTG |
| | TGTCAGGCTCACGGCTTGACTCCAGAACAGGTGGTGGCTATTGCTTCCCACG |
| | ACGGGGGGAAACAGGCCCTGGAAACTGTGCAGCGCCTGCTGCCAGTGCTGT |
| | GCCAGGCTCACGGGCTGACCCCCGAACAGGTGGTGGCCATTGCCAGCAACG |
| | GCGGCGGCAAGCAGGCCCTGGAAACCGTGCAGAGACTGCTGCCCGTGCTGT |
| | GCCAGGCCCATGGCCTGACACCTGAACAGGTGGTGGCTATCGCCTCTCACGA |
| | CGGAGGAAAACAGGCTCTGGAAACAGTGCAGCGGCTGCTGCCTGTGCTGTGT |
| | CAGGCTCACGGCTTGACTCCAGAACAGGTGGTGGCTATTGCTTCCAATATTG |
| | GGGGGAAACAGGCCCTGGAAACTGTGCAGCGCCTGCTGCCAGTGCTGTGCCA |
| | GGCTCACGGCCTCACTCCCGAACAGGTGGTGGCCATTGCCAGCAACGGCGGC |
| | GGCAAGCAGGCCCTGGAAACCGTGCAGAGACTGCTGCCCGTGCTGTGCCAG |
| | GCCCATGGCCTGACACCTGAACAGGTGGTGGCTATCGCCTCTCACGACGGAG |
| | GAAAACAGGCTCTGGAAACAGTGCAGCGGCTGCTGCCTGTGCTGTGTCAGGC |
| | TCACGGCTTGACTCCAGAACAGGTGGTGGCTATTGCTTCCAACAACGGGGGG |
| | AAACAGGCCCTGGAAACTGTGCAGCGCCTGCTGCCAGTGCTGTGCCAGGCTC |
| | ACGGACTGACCCCCGAACAGGTGGTGGCCATTGCCAGCCACGACGGCGGCA |
| | AGCAGGCCCTGGAAACCGTGCAGAGACTGCTGCCCGTGCTGTGCCAGGCCCA |
| | TGGCCTGACACCTGAACAGGTGGTGGCTATCGCCTCTCACGACGGAGGAAAA |
| | CAAGCACTCGAGACAGTGCAGCGGCTGCTGCCTGTGCTGTGTCAGGCTCACG |
| | GCTTGACTCCAGAACAGGTGGTGGCTATTGCTTCCAATATTGGGGGGAAACA |
| | GGCCCTGGAAACTGTGCAGCGCCTGCTGCCAGTGCTGTGCCAGGCTCACGGC |
| | CTGACCCCCGAACAGGTGGTGGCCATTGCCAGCAACATCGGCGGCAAGCAG |
| | GCCCTGGAAACCGTGCAGAGACTGCTGCCCGTGCTGTGCCAGGCCCATGGCC |
| | TGACACCTGAACAGGTGGTGGCTATCGCCTCTCACGACGGAGGAAAACAGG |
| | CTCTGGAAACAGTGCAGCGGCTGCTGCCTGTGCTGTGTCAGGCTCACGGCTT |
| | GACTCCACAGCAGGTCGTGGCAATTGCTAGCAATATCGGCGGACGGCCCGCC |
| | CTGGAGAGCATTGTGGCCCAGCTGTCTAGACCGGACCCAGCTCTCGCTGCAT |
| | TGACTAACGACCATCTCGTCGCTTTGGCTTGCCTCGGTGGAAGGCCAGCTCTT |
| | GACGCTGTAAAGAAAGGTTTGCCACATGCTCCAGCTCTTATTAAAAGGACTG |
| | GTAGCGGTAGCATGAATGAATTAACCTTGCACCCTAACGTCTACGCAACAGG |
| | AGCCTCCAAGGGTTTGATACACGTATTGGAAAGGGTGTGGGTAAGGGAGCAT |
| | ACTCCAGGTGATGGAACAATATTTGTCATTAGCGGATTCGGAAATTATAATG |
| | GAGGTGTAAGGTTCTTTGAATCCTTCAGGGAGCATAAACGCAGGGGAGGTGA |
| | ATTGGTTGCTGTCTTTGCAGGATCAACCTCGGCTAGGTTGACTTCTAAACAAG |
| | TAGTCCATGAAATGTTGGACTGTGGTGCAGACGTACATATTGTAAATCGCAA |
| | AAGGTTGCTTCATGCAAAATGCTATGGTACGTCCAAAATGAGCGGAGACAGC |
| | TTGATAGTCACAAGCGGTAACTTTACAGGGCCTGGTATGAGCCAAAATGTCG |
| | AGATGAGCGTGCTCTTGGATGCTGACACGACTGCGGAAATGGGATTTAGCTG |
| | GGGTACACTCTTGGAAAATCTGTTGGACCAAAGGTGGAATTATCACAGACCG |
| | ACGCTCGGCGACCCTGCGAACCCTGCATGGTCGTTGCTGTACGACGAACAAG |
| | CTGCGGACATACGCCTCGACGAGACAGAGGAATGA |
| 64 | MAAKIRIVMKSFMSQANKVEGVIPYAQKVGLPESRSLYTVLRSPHIDKKSREQFS |
| | MTGRATMGKPIPNPLLGLDSTGGVDLRTLGYSQQQQEKIKPKVRSTVAQHHEAL |
| | VGHGFTHAHIVALSQHPAALGTVAVKYQDMIAALPEATHEAIVGVGKRGAGAR |
| | ALEALLTVAGELRGPPLQLDTGQLLKIAKRGGVTAVEAVHAWRNALTGAPLNL |
| | TPEQVVAIASNNGGKQALETVQRLLPVLCQAHGLTPEQVVAIASHDGGKQALET |
| | VQRLLPVLCQAHGLTPEQVVAIASNIGGKQALETVQRLLPVLCQAHGLTPEQVV |
| | AIASNNGGKQALETVQRLLPVLCQAHGLTPEQVVAIASHDGGKQALETVQRLLP |
| | VLCQAHGLTPEQVVAIASHDGGKQALETVQRLLPVLCQAHGLTPEQVVAIASNG |
| | GGKQALETVQRLLPVLCQAHGLTPEQVVAIASHDGGKQALETVQRLLPVLCQA |
| | HGLTPEQVVAIASNIGGKQALETVQRLLPVLCQAHGLTPEQVVAIASNGGGKQA |
| | LETVQRLLPVLCQAHGLTPEQVVAIASHDGGKQALETVQRLLPVLCQAHGLTPE |
| | QVVAIASNNGGKQALETVQRLLPVLCQAHGLTPEQVVAIASHDGGKQALETVQ |
| | RLLPVLCQAHGLTPEQVVAIASHDGGKQALETVQRLLPVLCQAHGLTPEQVVAI |
| | ASNIGGKQALETVQRLLPVLCQAHGLTPEQVVAIASNIGGKQALETVQRLLPVLC |
| | QAHGLTPEQVVAIASHDGGKQALETVQRLLPVLCQAHGLTPQQVVAIASNIGGR |
| | PALESIVAQLSRPDPALAALTNDHLVALACLGGRPALDAVKKGLPHAPALIKRT |
| | GSGSMNELTLHPNVYATGASKGLIHVLERVWVREHTPGDGTIFVISGFGNYNGG |
| | VRFFESFREHKRRGGELVAVFAGSTSARLTSKQVVHEMLDCGADVHIVNRKRLL |
| | HAKCYGTSKMSGDSLIVTSGNFTGPGMSQNVEMSVLLDADTTAEMGFSWGTLL |
| | ENLLDQRWNYHRPTLGDPANPAWSLLYDEQAADIRLDETEE |
| 65 | ATGTTTAAACAAGCTTCCCGTCTTCTGTCACGCTCAGTTGCAGCTGCTTCCTC |
| | TAAGTCCGTCACCACCCGCGCTTTCTCCACCGAGCTGCCATCAACCCTGGACT |
| | CCACCGGCCGCGCCACCATGGGAAAACCGATCCCAAACCCGCTTTTGGGGCT |
| | CGACAGCACAGGAGGGGTTGATCTCAGGACCCTCGGTTACAGCCAACAGCA |
| | ACAAGAAAAAATTAAGCCTAAAGTGAGGTCCACGGTAGCTCAACATCACGA |
| | GGCTTTGGTAGGGCATGGCTTCACGCACGCTCATATCGTCGCTCTCAGCCAA |
| | CATCCCGCTGCGCTTGGGACGGTCGCTGTTAAGTACCAAGACATGATCGCTG |
| | CGCTTCCGGAAGCTACGCATGAGGCGATCGTCGGTGTAGGGAAGCGGGGAG |
| | CAGGTGCGAGGGCGCTCGAGGCGCTCTTGACCGTCGCGGGTGAGCTTAGGGG |
| | TCCGCCACTCCAATTGGACACCGGACAACTTTTGAAAATCGCTAAGAGAGGA |
| | GGCGTCACAGCCGTCGAGGCGGTGCACGCCTGGAGAAATGCCCTGACAGGA |
| | GCCCCTCTGAACCTGACCCCCGAACAGGTGGTGGCCATTGCCAGCCACGACG |
| | GCGGCAAGCAGGCCCTGGAAACCGTGCAGAGACTGCTGCCCGTGCTGTGCCA |
| | GGCCCATGGCCTGACACCTGAACAGGTGGTGGCTATCGCCTCTCACGACGGA |
| | GGAAAACAGGCTCTGGAAACAGTGCAGCGGCTGCTGCCTGTGCTGTGTCANG |
| | CTCACGGCTTGACTCCAGAACAGGTGGTGGCTATTGCTTCCAATATTGGGGG |
| | GAAACAGGCCCTGGAAACTGTGCAGCGCCTGCTGCCAGTGCTGTGCCAGGCT |
| | CACGGACTGACCCCCGAACAGGTGGTGGCCATTGCCAGCAACGGCGGCGGC |
| | AAGCAGGCCCTGGAAACCGTGCAGAGACTGCTGCCCGTGCTGTGCCAGGCCC |
| | ATGGCCTGACACCTGAACAGGTGGTGGCTATCGCCTCTAATATCGGAGGAAA |
| | ACAGGCTCTGGAAACAGTGCAGCGGCTGCTGCCTGTGCTGTGTCAGGCTCAC |
| | GGCTTGACTCCAGAACAGGTGGTGGCTATTGCTTCCAACAACGGGGGGAAAC |
| | AGGCCCTGGAAACTGTGCAGCGCCTGCTGCCAGTGCTGTTCCAGGCTCACGG |
| | GCTGACCCCCGAACAGGTGGTGGCCATTGCCAGCAACGGCGGCGGCAAGCA |
| | GGCCCTGGAAACCGTGCAGAGACTGCTGCCCGTGCTGTGCCAGGCCCATGGC |
| | CTGACACCTGAACAGGTGGTGGCTATCGCCTCTAATATCGGAGGAAAACAGG |
| | CTCTGGAAACAGTGCAGCGGCTGCTGCCTGTGCTGTGTCAGGCTCACGGCTT |
| | GACTCCAGAACAGGTGGTGGCTATTGCTTCCCACGACGGGGGGAAACAGGC |
| | CCTGGAAACTGTGCAGCGCCTGCTGCCAGTGCTGTGCCAGGCTCACGGCCTC |
| | ACTCCCGAACAGGTGGTGGCCATTGCCAGCAACATCGGCGGCAAGCAGGCC |
| | CTGGAAACCGTGCAGAGACTGCTGCCCGTGCTGTGCCAGGCCCATGGCCTGA |
| | CACCTGAACAGGTGGTGGCTATCGCCTCTAACGGCGGAGGAAAACAGGCTCT |
| | GGAAACAGTGCAGCGGCTGCTGCCTGTGCTGTGTCAGGCTCACGGCTTGACT |
| | CCAGAACAGGTGGTGGCTATTGCTTCCAACAACGGGGGGAAACAGGCCCTG |
| | GAAACTGTGCAGCGCCTGCTGCCAGTGCTGTGCCAGGCTCACGGACTGACCC |
| | CCGAACAGGTGGTGGCCATTGCCAGCCACGACGGCGGCAAGCAGGCCCTGG |
| | AAACCGTGCAGAGACTGCTGCCCGTGCTGTGCCAGGCCCATGGCCTGACACC |
| | TGAACAGGTGGTGGCTATCGCCTCTCACGACGGAGGAAAACAAGCACTCGA |
| | GACAGTGCAGCGGCTGCTGCCTGTGCTGTGTCAGGCTCACGGCTTGACTCCA |
| | GAACAGGTGGTGGCTATTGCTTCCAACAACGGGGGGAAACAGGCCCTGGAA |
| | ACTGTGCAGCGCCTGCTGCCAGTGCTGTGCCAGGCTCACGGCCTGACCCCCG |
| | AACAGGTGGTGGCCATTGCCAGCAACATCGGCGGCAAGCAGGCCCTGGAAA |
| | CCGTGCAGAGACTGCTGCCCGTGCTGTGCCAGGCCCATGGCCTGACACCTGA |
| | ACAGGTGGTGGCTATCGCCTCTAACGGCGGAGGAAAACAGGCTCTGGAAAC |
| | AGTGCAGCGGCTGCTGCCTGTGCTGTGTCAGGCTCACGGCTTGACTCCACAG |
| | CAGGTCGTGGCAATTGCTAGCAACGGCGGCGGACGGCCCGCCCTGGAGAGC |
| | ATTGTGGCCCAGCTGTCTAGACCTGATCCTGCCCTGGCCGCCCTGACAAATG |
| | ATCACCTGGTGGCCCTGGCCTGTCTGGGAGGCAGACCTGCCCTGGATGCCGT |
| | GAAAAAAGGACTGCCTCACGCCCCTGCCCTGATCAAGAGAACAGGATCCGG |
| | ATCCATGAACGAGTTAACGCTGCATCCGAATGTGTATGCGACCGGCGCGAGT |
| | AAAGGGCTGATACATGTGCTGGAGCGCGTATGGGTTCGAGAACACACGCCTG |
| | GGGACGGCACGATCTTCGTGATATCTGGCTTTGGCAACTACAACGGTGGCGT |
| | TCGCTTCTTCGAGTCGTTTCGTGAACACAAGAGACGTGGCGGGGAGCTGGTA |
| | GCGGTGTTCGCTGGCAGCACAAGCGCGCGACTGACAAGCAAACAGGTTGTTC |
| | ACGAGATGCTTGATTGCGGGGCGGATGTTCACATAGTGAACAGAAAGCGACT |
| | CCTTCACGCTAAGTGTTACGGCACAAGCAAGATGTCAGGTGATTCCCTAATT |
| | GTAACCTCAGGCAATTTCACAGGTCCGGGAATGTCGCAGAACGTGGAAATGT |
| | CTGTATTGCTAGACGCAGATACAACCGCCGAGATGGGGTTCTCGTGGGGCAC |
| | GTTGCTTGAGAACTTGCTTGATCAGCGATGGAACTACCATCGCCCCACCTTA |
| | GGCGATCCAGCAAATCCAGCCTGGAGCCTGCTTTATGATGAGCAGGCAGCCG |
| | ATATTAGGTTGGATGAAACTGAAGAATGA |
| 66 | MFKQASRLLSRSVAAASSKSVTTRAFSTELPSTLDSTGRATMGKPIPNPLLGLDST |
| | GGVDLRTLGYSQQQQEKIKPKVRSTVAQHHEALVGHGFTHAHIVALSQHPAAL |
| | GTVAVKYQDMIAALPEATHEAIVGVGKRGAGARALEALLTVAGELRGPPLQLD |
| | TGQLLKIAKRGGVTAVEAVHAWRNALTGAPLNLTPEQVVAIASHDGGKQALET |
| | VQRLLPVLCQAHGLTPEQVVAIASHDGGKQALETVQRLLPVLCXAHGLTPEQV |
| | VAIASNIGGKQALETVQRLLPVLCQAHGLTPEQVVAIASNGGGKQALETVQRLL |
| | PVLCQAHGLTPEQVVAIASNIGGKQALETVQRLLPVLCQAHGLTPEQVVAIASN |
| | NGGKQALETVQRLLPVLFQAHGLTPEQVVAIASNGGGKQALETVQRLLPVLCQ |
| | AHGLTPEQVVAIASNIGGKQALETVQRLLPVLCQAHGLTPEQVVAIASHDGGKQ |
| | ALETVQRLLPVLCQAHGLTPEQVVAIASNIGGKQALETVQRLLPVLCQAHGLTP |
| | EQVVAIASNGGGKQALETVQRLLPVLCQAHGLTPEQVVAIASNNGGKQALETV |
| | QRLLPVLCQAHGLTPEQVVAIASHDGGKQALETVQRLLPVLCQAHGLTPEQVV |
| | AIASHDGGKQALETVQRLLPVLCQAHGLTPEQVVAIASNNGGKQALETVQRLLP |
| | VLCQAHGLTPEQVVAIASNIGGKQALETVQRLLPVLCQAHGLTPEQVVAIASNG |
| | GGKQALETVQRLLPVLCQAHGLTPQQVVAIASNGGGRPALESIVAQLSRPDPAL |
| | AALTNDHLVALACLGGRPALDAVKKGLPHAPALIKRTGSGSMNELTLHPNVYA |
| | TGASKGLIHVLERVWVREHTPGDGTIFVISGFGNYNGGVRFFESFREHKRRGGEL |
| | VAVFAGSTSARLTSKQVVHEMLDCGADVHIVNRKRLLHAKCYGTSKMSGDSLI |
| | VTSGNFTGPGMSQNVEMSVLLDADTTAEMGFSWGTLLENLLDQRWNYHRPTL |
| | GDPANPAWSLLYDEQAADIRLDETEE |
| 67 | AGACATATATATCTATCTAAATTTATTGATATCAATATGAATGTGGAAAATG |
| | CTAAAATAACTTACATTGTGAAATGAAGGAAGTAATTTTCTTCACAAATTTTG |
| | GCACTTGCACTTACGGCTTGTGAAGCCCGCTCGATCTGAAGATTTTTCAAACG |
| | ATGCCATTCACCACGTGCCACTTTATATCAAACCTTATTTGTATCCAAGATTT |
| | CTGGTACTGGGCATTAGCGATGTCAAACAAACTCGACGTAAAGTTGAGGGAC |
| | AGAGTGTGAACTTATTTTATTCCCAGCAGAGAACGCCTCCAAGGTTTATGGG |
| | CTTTCTGGTCCATCGCAACGACACGGACTTTCTACAGGGCTGAGATTACGGG |
| | CCTAAATGGGCTCCCCCGTCTTGTGCTCCGCGCTGTTTCAAGTTGGGCCAAAA |
| | TGGAACTGGGCCGAATAGTCCGGTGCGGCTAACGGCATCCACAGCCAAGCC |
| | AACGGTGCCGTTCTCCCGCTCTCGCATTTCAAAAATCGAAAATAAAATCGGC |
| | AGGGCCGCCGACGACGCAACCACCCGTTGGGATCCAATCTACTCCTACGCAC |
| | CGAACAACTCATCGCACCCACCGATATGTGGGACCCAGCTGTAACGTGGGCC |
| | CACCGGCAAGCGGGACGTCTGCATCGGTCGCACCTACAACGAGGTGGCCAG |
| | GTCACCTACTGCTTTTTCTTGGAGCGACGCGTGCACTCGCTAACCGCACACGA |
| | AGGAAGCCTCTGGACCCACCTGTCAGTGAAACTAACGAGAGGCGGGTATGG |
| | ACTTCAACCGAGTGCAAACGAAATGGCCTCCTTGCGGTGGATATTATCAGAA |
| | AGCTCCCTAACTTATTAAATATTCTCGATATGATCCAACCACCCGACCTTTGC |
| | AGGCACGATGTGAGATGCGAGTGAATTTTGAAGACGTCAAAGGGGGTAAAA |
| | CGAAAAACTCCCCCATCCCTTCGCTTCCGCTCCTGAGAGGTCCAGGCTGTCTG |
| | GCCCTATAAAACGGGCGGAGTGCGGGGATCGCAAACCCTAAGCCGAGACAA |
| | CGCAGAGAAAGGCGTCTTCGTACTCGCCTCTCTCCGCGCCTCCGCGCTTTTCC |
| | TCCTCTCCCCTCTCTCCCTTCTCCGCCGCCGTCGCAGCATCAACCCAATCCGC |
| | CGCCAAGCTTCATCCAGGTACGGATCCGCGTCCCATCTCCCTCACCCCCCGTG |
| | TTCTTCGTGCCTGCTTCTGGGTCAGATCTGGGTGGATTCGCGGTTGTTGGATG |
| | TGGGGGGCTGTGTTTATTTGTCGGTGGATCTGGTTGTCTGGATCTGCGTTTTC |
| | TCTGTCGTAGTTAGCGGATCTGATGAAATGTTTAGTGTTCGTGTATACTGGTA |
| | TGGTGGATCTGGTCCTAGGATGCGTGGAATGGATATATGTAGGCGAATTGGA |
| | GGATTTATTTTGTGAATTTTGCTGAAATGATAGTTCTAAACACTGGATCTGAC |
| | CTCGGGATGCTGTTAAATGTGGAAATCATGGTCGATGCTGTCATGAACATGG |
| | TGTTCTTATGGTAGATCTGAGCAATGTATGTTTCAAAATTGTTTGTCACATGG |
| | AAATGCTATGGTTCTAGATGCAATAGAATGATACATGCCGAGATCCCCTCTA |
| | GTTGATATGATAGATCATGATGTTTTACAGCTATGTCATATGAATATGTTCAT |
| | TTGTTACCGATGTATTTGGATCTACTTAACATTTCCAAAGCACGCCGCGTTCT |
| | AATTCTAGATCTGGTAGTCATGTTTGTACACGTCATCCACCTAATACAAATAC |
| | ATATGTCTAGTGTTTGGTGACACTGCCCGTCAGATCTGTTTTTTCCAGATCTG |
| | TGGAACAAATACTCCATGCATGTATGGTAGTTTTGAAACGATCTTGTATCTTC |
| | CATTGTTGTAGTAACAACTAAATAAAGTACAATTGTTCAATTATTGGGAATC |
| | GTATTTTCTGTAGTGCCGATGTACAGCATATTCATAGATGTCTATTTAGGAAC |
| | TCAAATTTTAAATTGAGGACTAGTTATTTATTATGGGTCAGTCTTTTGAATTG |
| | TGTTATCTTGCTGTACTGAAATAATAATGTACCACTAAGGCGCTAACATGTAT |
| | TTTGTCCTTTCAGGCTGACTCGACGGATCCCGGGGGGCAAGAGCTCCTCCCC |
| | CCTCCCCCTCCGCCGCCGCCGGTAACCACCCCGCCCCTCTCCTCTTTCTTTCTC |
| | CGTTTTTTTTTTCGTCTCGGTCTCGATCTTTGGCCTTGGTAGTTTGGGTGGGCG |
| | AGAGCGGCTTCGTCGCCCAGATCGGTGCGCGGGAGGGGCGGGATCTCGCGG |
| | CTGGCGTCTCCGGGCGTGAGTCGGCCCGGATCCTCGCGGGGAATGGGGCTCT |
| | CGGATGTAGATCTTCTTTCTTTCTTCTTTTTGTGGTAGAATTTGAATCCCTCAG |
| | CATTGTTCATCGGTAGTTTTTCTTTTCATGATTTGTGACAAATGCAGCCTCGT |
| | GCGGAGCTTTTTTGTAGGTAGAAGATGGCAGCGAAGATCCGGATCGTCAT |
| | GAAGTCCTTCATGAGCCAGGCCAATAAGGTGGAGGGCGTCATCCCGTAT |
| | GCCCAAAAAGTCGGGCTCCCAGAGTCGCGCTCTCTCTACACAGTCTTGC |
| | GGTCGCCACATATCGATAAAAAGTCCAGAGAACAATTCTCTATGACCGG |
| | CCGCGCCACCATGGGAAAACCTATTCCTAATCCTCTGCTGGGCCTGGAT |
| | TCTACCGGAGGCGTGGACCTGAGAACACTGGGATATTCTCAGCAGCAGC |
| | AGGAGAAGATCAAGCCCAAGGTGAGATCTACAGTGGCCCAGCACCACG |
| | AAGCCCTGGTGGGACACGGATTTACACACGCCCACATTGTGGCCCTGTC |
| | TCAGCACCCTGCCGCCCTGGGAACAGTGGCCGTGAAATATCAGGATATG |
| | ATTGCCGCCCTGCCTGAGGCCACACACGAAGCCATTGTGGGAGTGGGAA |
| | AACGAGGCGCTGGAGCCAGAGCCCTGGAAGCCCTGCTGACAGTGGCCG |
| | GAGAACTGAGAGGACCTCCTCTGCAGCTGGATACAGGACAGCTGCTGAA |
| | GATTGCCAAAAGGGGCGGAGTGACCGCGGTGGAAGCCGTGCACGCCTG |
| | GAGAAATGCCCTGACAGGAGCCCCTCTGAACCTGACCCCCGAACAGGTG |
| | GTGGCCATTGCCAGCAACAACGGCGGCAAGCAGGCCCTGGAAACCGTG |
| | CAGAGACTGCTGCCCGTGCTGTGCCAGGCCCATGGCCTGACACCTGAAC |
| | AGGTGGTGGCTATCGCCTCTCACGACGGAGGAAAACAGGCTCTGGAAAC |
| | AGTGCAGCGGCTGCTGCCTGTGCTGTGTCAGGCTCACGGCTTGACTCCA |
| | GAACAGGTGGTGGCTATTGCTTCCAATATTGGGGGGAAACAGGCCCTGG |
| | AAACTGTGCAGCGCCTGCTGCCAGTGCTGTGCCAGGCTCACGGACTGAC |
| | CCCCGAACAGGTGGTGGCCATTGCCAGCAACAACGGCGGCAAGCAGGC |
| | CCTGGAAACCGTGCAGAGACTGCTGCCCGTGCTGTGCCAGGCCCATGGC |
| | CTGACACCTGAACAGGTGGTGGCTATCGCCTCTCACGACGGAGGAAAAC |
| | AGGCTCTGGAAACAGTGCAGCGGCTGCTGCCTGTGCTGTGTCAGGCTCA |
| | CGGCTTGACTCCAGAACAGGTGGTGGCTATTGCTTCCCACGACGGGGGG |
| | AAACAGGCCCTGGAAACTGTGCAGCGCCTGCTGCCAGTGCTGTGCCAGG |
| | CTCACGGGCTGACCCCCGAACAGGTGGTGGCCATTGCCAGCAACGGCG |
| | GCGGCAAGCAGGCCCTGGAAACCGTGCAGAGACTGCTGCCCGTGCTGT |
| | GCCAGGCCCATGGCCTGACACCTGAACAGGTGGTGGCTATCGCCTCTCA |
| | CGACGGAGGAAAACAGGCTCTGGAAACAGTGCAGCGGCTGCTGCCTGT |
| | GCTGTGTCAGGCTCACGGCTTGACTCCAGAACAGGTGGTGGCTATTGCT |
| | TCCAATATTGGGGGGAAACAGGCCCTGGAAACTGTGCAGCGCCTGCTGC |
| | CAGTGCTGTGCCAGGCTCACGGCCTCACTCCCGAACAGGTGGTGGCCAT |
| | TGCCAGCAACGGCGGCGGCAAGCAGGCCCTGGAAACCGTGCAGAGACT |
| | GCTGCCCGTGCTGTGCCAGGCCCATGGCCTGACACCTGAACAGGTGGTG |
| | GCTATCGCCTCTCACGACGGAGGAAAACAGGCTCTGGAAACAGTGCAGC |
| | GGCTGCTGCCTGTGCTGTGTCAGGCTCACGGCTTGACTCCAGAACAGGT |
| | GGTGGCTATTGCTTCCAACAACGGGGGGAAACAGGCCCTGGAAACTGTG |
| | CAGCGCCTGCTGCCAGTGCTGTGCCAGGCTCACGGACTGACCCCCGAAC |
| | AGGTGGTGGCCATTGCCAGCCACGACGGCGGCAAGCAGGCCCTGGAAA |
| | CCGTGCAGAGACTGCTGCCCGTGCTGTGCCAGGCCCATGGCCTGACACC |
| | TGAACAGGTGGTGGCTATCGCCTCTCACGACGGAGGAAAACAAGCACTC |
| | GAGACAGTGCAGCGGCTGCTGCCTGTGCTGTGTCAGGCTCACGGCTTGA |
| | CTCCAGAACAGGTGGTGGCTATTGCTTCCAATATTGGGGGGAAACAGGC |
| | CCTGGAAACTGTGCAGCGCCTGCTGCCAGTGCTGTGCCAGGCTCACGGC |
| | CTGACCCCCGAACAGGTGGTGGCCATTGCCAGCAACATCGGCGGCAAG |
| | CAGGCCCTGGAAACCGTGCAGAGACTGCTGCCCGTGCTGTGCCAGGCC |
| | CATGGCCTGACACCTGAACAGGTGGTGGCTATCGCCTCTCACGACGGAG |
| | GAAAACAGGCTCTGGAAACAGTGCAGCGGCTGCTGCCTGTGCTGTGTCA |
| | GGCTCACGGCTTGACTCCACAGCAGGTCGTGGCAATTGCTAGCAATATC |
| | GGCGGACGGCCCGCCCTGGAGAGCATTGTGGCCCAGCTGTCTAGACCG |
| | GACCCAGCTCTCGCTGCATTGACTAACGACCATCTCGTCGCTTTGGCTT |
| | GCCTCGGTGGAAGGCCAGCTCTTGACGCTGTAAAGAAAGGTTTGCCACA |
| | TGCTCCAGCTCTTATTAAAAGGACTGGTAGCGGTAGCATGAATGAATTA |
| | ACCTTGCACCCTAACGTCTACGCAACAGGAGCCTCCAAGGGTTTGATAC |
| | ACGTATTGGAAAGGGTGTGGGTAAGGGAGCATACTCCAGGTGATGGAA |
| | CAATATTTGTCATTAGCGGATTCGGAAATTATAATGGAGGTGTAAGGTT |
| | CTTTGAATCCTTCAGGGAGCATAAACGCAGGGGAGGTGAATTGGTTGCT |
| | GTCTTTGCAGGATCAACCTCGGCTAGGTTGACTTCTAAACAAGTAGTCC |
| | ATGAAATGTTGGACTGTGGTGCAGACGTACATATTGTAAATCGCAAAAG |
| | GTTGCTTCATGCAAAATGCTATGGTACGTCCAAAATGAGCGGAGACAGC |
| | TTGATAGTCACAAGCGGTAACTTTACAGGGCCTGGTATGAGCCAAAATG |
| | TCGAGATGAGCGTGCTCTTGGATGCTGACACGACTGCGGAAATGGGATT |
| | TAGCTGGGGTACACTCTTGGAAAATCTGTTGGACCAAAGGTGGAATTAT |
| | CACAGACCGACGCTCGGCGACCCTGCGAACCCTGCATGGTCGTTGCTGT |
| | ACGACGAACAAGCTGCGGACATACGCCTCGACGAGACAGAGGAATGAG |
| | AATTCATATGAAGATGAAGATGAAATATTTGGTGTGTCAAATAAAAAGCTTG |
| | TGTGCTTAAGTTTGTGTTTTTTTCTTGGCTTGTTGTGTTATGAATTTGTGGCTT |
| | TTTCTAATATTAAATGAATGTAAGATCTCATTATAATGAATAAACAAATGTTT |
| | CTATAATCCATTGTGAATGTTTTGTTGGATCTCTTCTGCAGCATATAACTACT |
| | GTATGTGCTATGGTATGGACTATGGAATATGATTAAAGATAAG |
| 68 | CTGCAGTGCAGCGTGACCCGGTCGTGCCCCTCTCTAGAGATAATGAGCATTG |
| | CATGTCTAAGTTATAAAAAATTACCACATATTTTTTTTGTCACACTTGTTTGA |
| | AGTGCAGTTTATCTATCTTTATACATATATTTAAACTTTACTCTACGAATAAT |
| | ATAATCTATAGTACTACAATAATATCAGTGTTTTAGAGAATCATATAAATGA |
| | ACAGTTAGACATGGTCTAAAGGACAATTGAGTATTTTGACAACAGGACTCTA |
| | CAGTTTTATCTTTTTAGTGTGCATGTGTTCTCCTTTTTTTTTGCAAATAGCTTC |
| | ACCTATATAATACTTCATCCATTTTATTAGTACATCCATTTAGGGTTTAGGGT |
| | TAATGGTTTTTATAGACTAATTTTTTTAGTACATCTATTTTATTCTATTTTAGC |
| | CTCTAAATTAAGAAAACTAAAACTCTATTTTAGTTTTTTTATTTAATAATTTA |
| | GATATAAAATAGAATAAAATAAAGTGACTAAAAATTAAACAAATACCCTTTA |
| | AGAAATTAAAAAAACTAAGGAAACATTTTTCTTGTTTCGAGTAGATAATGCC |
| | AGCCTGTTAAACGCCGTCGACGAGTCTAACGGACACCAACCAGCGAACCAG |
| | CAGCGTCGCGTCGGGCCAAGCGAAGCAGACGGCACGGCATCTCTGTCGCTGC |
| | CTCTGGACCCCTCTCGAGAGTTCCGCTCCACCGTTGGACTTGCTCCGCTGTCG |
| | GCATCCAGAAATTGCGTGGCGGAGCGGCAGACGTGAGCCGGCACGGCAGGC |
| | GGCCTCCTCCTCCTCTCACGGCACCGGCAGCTACGGGGGATTCCTTTCCCACC |
| | GCTCCTTCGCTTTCCCTTCCTCGCCCGCCGTAATAAATAGACACCCCCTCCAC |
| | ACCCTCTTTCCCCAACCTCGTGTTGTTCGGAGCGCACACACACACAACCAGA |
| | TCTCCCCCAAATCCACCCGTCGGCACCTCCGCTTCAAGGTACGCCGCTCGTCC |
| | TCCCCCCCCCCCCCTCTCTACCTTCTCTAGATCGGCGTTCCGGTCCATGGTTA |
| | GGGCCCGGTAGTTCTACTTCTGTTCATGTTTGTGTTAGATCCGTGTTTGTGTTA |
| | GATCCGTGCTGCTAGCGTTCGTACACGGATGCGACCTGTACGTCAGACACGT |
| | TCTGATTGCTAACTTGCCAGTGTTTCTCTTTGGGGAATCCTGGGATGGCTCTA |
| | GCCGTTCCGCAGACGGGATCGATTTCATGATTTTTTTTGTTTCGTTGCATAGG |
| | GTTTGGTTTGCCCTTTTCCTTTATTTCAATATATGCCGTGCACTTGTTTGTCGG |
| | GTCATCTTTTCATGCTTTTTTTTGTCTTGGTTGTGATGATGTGGTCTGGTTGGG |
| | CGGTCGTTCTAGATCGGAGTAGAATTAATTCTGTTTCAAACTACCTGGTGGAT |
| | TTATTAATTTTGGATCTGTATGTGTGTGCCATACATATTCATAGTTACGAATT |
| | GAAGATGATGGATGGAAATATCGATCTAGGATAGGTATACATGTTGATGCGG |
| | GTTTTACTGATGCATATACAGAGATGCTTTTTGTTCGCTTGGTTGTGATGATG |
| | TGGTGTGGTTGGGCGGTCGTTCATTCGTTCTAGATCGGAGTAGAATACTGTTT |
| | CAAACTACCTGGTGTATTTATTAATTTTGGAACTGTATGTGTGTGTCATACAT |
| | CTTCATAGTTACGAGTTTAAGATGGATGGAAATATCGATCTAGGATAGGTAT |
| | ACATGTTGATGTGGGTTTTACTGATGCATATACATGATGGCATATGCAGCATC |
| | TATTCATATGCTCTAACCTTGAGTACCTATCTATTATAATAAACAAGTATGTT |
| | TTATAATTATTTTGATCTTGATATACTTGGATGATGGCATATGCAGCAGCTAT |
| | ATGTGGATTTTTTTAGCCCTGCCTTCATACGCTATTTATTTGCTTGGTACTGTT |
| | TCTTTTGTCGATGCTCACCCTGTTGTTTGGTGTTACTTCTGCAGGTCGACTCTA |
| | GAGGATCCATGTTTAAACAAGCTTCCCGTCTTCTGTCACGCTCAGTTGCA |
| | GCTGCTTCCTCTAAGTCCGTCACCACCCGCGCTTTCTCCACCGAGCTGC |
| | CATCAACCCTGGACTCCACCGGCCGCGCCACCATGGGAAAACCGATCCC |
| | AAACCCGCTTTTGGGGCTCGACAGCACAGGAGGGGTTGATCTCAGGACC |
| | CTCGGTTACAGCCAACAGCAACAAGAAAAAATTAAGCCTAAAGTGAGGT |
| | CCACGGTAGCTCAACATCACGAGGCTTTGGTAGGGCATGGCTTCACGCA |
| | CGCTCATATCGTCGCTCTCAGCCAACATCCCGCTGCGCTTGGGACGGTC |
| | GCTGTTAAGTACCAAGACATGATCGCTGCGCTTCCGGAAGCTACGCATG |
| | AGGCGATCGTCGGTGTAGGGAAGCGGGGAGCAGGTGCGAGGGCGCTCG |
| | AGGCGCTCTTGACCGTCGCGGGTGAGCTTAGGGGTCCGCCACTCCAATT |
| | GGACACCGGACAACTTTTGAAAATCGCTAAGAGAGGAGGCGTCACAGCC |
| | GTCGAGGCGGTGCACGCCTGGAGAAATGCCCTGACAGGAGCCCCTCTG |
| | AACCTGACCCCCGAACAGGTGGTGGCCATTGCCAGCCACGACGGCGGC |
| | AAGCAGGCCCTGGAAACCGTGCAGAGACTGCTGCCCGTGCTGTGCCAG |
| | GCCCATGGCCTGACACCTGAACAGGTGGTGGCTATCGCCTCTCACGACG |
| | GAGGAAAACAGGCTCTGGAAACAGTGCAGCGGCTGCTGCCTGTGCTGT |
| | GTCANGCTCACGGCTTGACTCCAGAACAGGTGGTGGCTATTGCTTCCAA |
| | TATTGGGGGGAAACAGGCCCTGGAAACTGTGCAGCGCCTGCTGCCAGT |
| | GCTGTGCCAGGCTCACGGACTGACCCCCGAACAGGTGGTGGCCATTGCC |
| | AGCAACGGCGGCGGCAAGCAGGCCCTGGAAACCGTGCAGAGACTGCTG |
| | CCCGTGCTGTGCCAGGCCCATGGCCTGACACCTGAACAGGTGGTGGCTA |
| | TCGCCTCTAATATCGGAGGAAAACAGGCTCTGGAAACAGTGCAGCGGCT |
| | GCTGCCTGTGCTGTGTCAGGCTCACGGCTTGACTCCAGAACAGGTGGTG |
| | GCTATTGCTTCCAACAACGGGGGGAAACAGGCCCTGGAAACTGTGCAGC |
| | GCCTGCTGCCAGTGCTGTTCCAGGCTCACGGGCTGACCCCCGAACAGGT |
| | GGTGGCCATTGCCAGCAACGGCGGCGGCAAGCAGGCCCTGGAAACCGT |
| | GCAGAGACTGCTGCCCGTGCTGTGCCAGGCCCATGGCCTGACACCTGAA |
| | CAGGTGGTGGCTATCGCCTCTAATATCGGAGGAAAACAGGCTCTGGAAA |
| | CAGTGCAGCGGCTGCTGCCTGTGCTGTGTCAGGCTCACGGCTTGACTCC |
| | AGAACAGGTGGTGGCTATTGCTTCCCACGACGGGGGGAAACAGGCCCT |
| | GGAAACTGTGCAGCGCCTGCTGCCAGTGCTGTGCCAGGCTCACGGCCTC |
| | ACTCCCGAACAGGTGGTGGCCATTGCCAGCAACATCGGCGGCAAGCAG |
| | GCCCTGGAAACCGTGCAGAGACTGCTGCCCGTGCTGTGCCAGGCCCATG |
| | GCCTGACACCTGAACAGGTGGTGGCTATCGCCTCTAACGGCGGAGGAAA |
| | ACAGGCTCTGGAAACAGTGCAGCGGCTGCTGCCTGTGCTGTGTCAGGCT |
| | CACGGCTTGACTCCAGAACAGGTGGTGGCTATTGCTTCCAACAACGGGG |
| | GGAAACAGGCCCTGGAAACTGTGCAGCGCCTGCTGCCAGTGCTGTGCCA |
| | GGCTCACGGACTGACCCCCGAACAGGTGGTGGCCATTGCCAGCCACGA |
| | CGGCGGCAAGCAGGCCCTGGAAACCGTGCAGAGACTGCTGCCCGTGCT |
| | GTGCCAGGCCCATGGCCTGACACCTGAACAGGTGGTGGCTATCGCCTCT |
| | CACGACGGAGGAAAACAAGCACTCGAGACAGTGCAGCGGCTGCTGCCT |
| | GTGCTGTGTCAGGCTCACGGCTTGACTCCAGAACAGGTGGTGGCTATTG |
| | CTTCCAACAACGGGGGGAAACAGGCCCTGGAAACTGTGCAGCGCCTGCT |
| | GCCAGTGCTGTGCCAGGCTCACGGCCTGACCCCCGAACAGGTGGTGGC |
| | CATTGCCAGCAACATCGGCGGCAAGCAGGCCCTGGAAACCGTGCAGAG |
| | ACTGCTGCCCGTGCTGTGCCAGGCCCATGGCCTGACACCTGAACAGGTG |
| | GTGGCTATCGCCTCTAACGGCGGAGGAAAACAGGCTCTGGAAACAGTGC |
| | AGCGGCTGCTGCCTGTGCTGTGTCAGGCTCACGGCTTGACTCCACAGCA |
| | GGTCGTGGCAATTGCTAGCAACGGCGGCGGACGGCCCGCCCTGGAGAG |
| | CATTGTGGCCCAGCTGTCTAGACCTGATCCTGCCCTGGCCGCCCTGACA |
| | AATGATCACCTGGTGGCCCTGGCCTGTCTGGGAGGCAGACCTGCCCTGG |
| | ATGCCGTGAAAAAAGGACTGCCTCACGCCCCTGCCCTGATCAAGAGAAC |
| | AGGATCCGGATCCATGAACGAGTTAACGCTGCATCCGAATGTGTATGCG |
| | ACCGGCGCGAGTAAAGGGCTGATACATGTGCTGGAGCGCGTATGGGTT |
| | CGAGAACACACGCCTGGGGACGGCACGATCTTCGTGATATCTGGCTTTG |
| | GCAACTACAACGGTGGCGTTCGCTTCTTCGAGTCGTTTCGTGAACACAA |
| | GAGACGTGGCGGGGAGCTGGTAGCGGTGTTCGCTGGCAGCACAAGCGC |
| | GCGACTGACAAGCAAACAGGTTGTTCACGAGATGCTTGATTGCGGGGCG |
| | GATGTTCACATAGTGAACAGAAAGCGACTCCTTCACGCTAAGTGTTACG |
| | GCACAAGCAAGATGTCAGGTGATTCCCTAATTGTAACCTCAGGCAATTT |
| | CACAGGTCCGGGAATGTCGCAGAACGTGGAAATGTCTGTATTGCTAGAC |
| | GCAGATACAACCGCCGAGATGGGGTTCTCGTGGGGCACGTTGCTTGAGA |
| | ACTTGCTTGATCAGCGATGGAACTACCATCGCCCCACCTTAGGCGATCC |
| | AGCAAATCCAGCCTGGAGCCTGCTTTATGATGAGCAGGCAGCCGATATT |
| | AGGTTGGATGAAACTGAAGAATGAGAATTCGGTACCGAGTTTGAATCAAA |
| | TCTTCACTTGTTTAATGAGATATGCGAGACGCCTATGATCGCATGATATTTGC |
| | TTTCAATTCTGTTGTGCACGTTGTAAAAAACCTGAGCATGTGTAGCTCAGATC |
| | CTTACCGCCGGTTTCGGTTCATTCTAATGAATATATCACCCGTTACTATCGTA |
| | TTTTTATGAATAATATTCTCCGTTCAATTTACTGATTGTACCCTACTACTTATA |
| | TGTACAATATTAAAATGAAAACAATATATTGTGCTGAATAGGTTTATAGCGA |
| | CATCTATGATAGAGCGCCACAATAACAAACAATTGCGTTTTATTATTACAAA |
| | TCCAATTTTAAAAAAAGCGGCAGAACCGGTCAAACCTAAAAGACTGATTACA |
| | TAAATCTTATTCAAATTTCAAAAGTGCCCCAGGGGCTAGTATCTACGACACA |
| | CCGAGCGGCGAATTCAGTACATTAAAAACGTCCGCAATGTGTTATTAAGTTG |
| | TCTAAGCGTCAATTTGTTTACACCACAATATATCCTGCCACCAGCCAGCCAAC |
| | AGCTCCCCGACCGGCAGCTCGGCACAAAATCACCACTCGATACAGGCAGCCC |
| | ATCAGTCCGGGACGGCGTCAGCGGGA |
| 69 | CCACCTGTCTCTCTTTTTTTTTCTTTGCTTTCTCAGATATTTCATAAAAAGGAC |
| | TCTCTACGGATCCCCACCAATGATCAATTCTCGCATGAATAGCTAAAGATCC |
| | AGTAAGTCCAACGTTGATTCCTTCGGACGTGTCAATTGGACAAATACGCCCA |
| | TAGTGACTCGGATGGATATCTCGGCTCCGAAAACTTGCAGTTCTCCCCGTCA |
| | ATCCTCCAGGACCCAAACAACTCACTTTTCGCCCATGAACCGTTTGTGTCAAT |
| | GGATTGGTTTGATCAAAAACTTGAGATAAGGGATATGTACCAAAGAAGGTCT |
| | CGTAAGTAGTTATTAATAAAATCGAGGTTGAAGTTGGAGTTACCAAAGTTTG |
| | TGGAGTCGGTTTTGATTGACGTATGAATACTCTACGGATAGTTTTTTGAACCG |
| | CATGTTGTAAACGACCAAGAGCCAGTCCGAATTGATCTTGTAACAGATCCGC |
| | AACCGAACGAATACGTTTATTTTTCAAGTGATTCATATCGTCATCGTCAAGTA |
| | TACCCGTTTCAAATTTCATTCCAATCAAATGATCCGTAGCGGCCAATACATCT |
| | CGCGGTAACAAGAAGGTGCTGTTCTGAGGTATATCAAGATTCAGTCTTCGAT |
| | TCATATTTCGTCGACCAATCCTTCCTAATTCACATTTTTGTTGAAAAAATTTCT |
| | TTTGTAATTCCTCACATAAGGATTCCGAAAATACCAGGTCCCCACCTACACA |
| | AGCAAATTGTTGATAAAACTCCAAAATAGCTTTTTCTTTTGACTCAATCCTCT |
| | TCTTCTCCTTAGCATTAGGGAAAGACAAGAAAATTTCAGGGTAGGAAACATT |
| | ATCTAGAATTTCTTTTAGATTTGAACCCATAGCTGATGATAGAACTAGAATA |
| | GATATCTTTTGTTTTCTACTCACGCGAGCCCATATCCTTTCTTTTTTATCAATT |
| | GCTAATTCCGATCTTCCTCCCCAATCTGATATTATAGTCCCGGTGTAGATAGA |
| | AATTCCTTTATGGTCTAATTCCGAGCGGTAGTAAATACCAGGACTTAGCAAT |
| | ATTTGATTGATCACAATTCGGTATATTCCATTTATTATAAAGGTTCCTAAGGA |
| | ATTCATTATAGGAATGTTTCCAATAGAAATGGTTTGCTTTTGCACATCGAAAC |
| | CAAAAATTAATCGCGCGGATACGTATAATTCGGAAGAATAGGTGAGTGATTC |
| | ATACACAGCATCCCTTTCTTTTATCGAAGGTTCTAGCAATTGATATCCTTTCG |
| | CAAATAATTGAAATGAAATTTCGTGATCTGGATCTTTAATTGTTGGAAACTTC |
| | TCAAGTTCTTCTGCCAAGCCTTGATTAATGAACCTACAAAATCCCTCGAATTG |
| | GATCTGACTAAATCCGGGTATTGTGGACATTCCCTCATTTCCATTCCGGAGCA |
| | TCTTAATCTTAAGTTTCCTGTTTATCGAAGAAAAATTCCATTATCAGCTCACT |
| | CTTCATCAATCCCTACAGATCGATCTAGCAATTATGGAATCTATATTCTGTTT |
| | ACTAAATCACATGAAATTCTCGGGAACTCCACATACGTATTTCATATATGTAT |
| | TTCATACATATGAATAGAGACAATTTCAACGAAAGTTCGAATTTCCCAGGGG |
| | TCCAAATGGAATGAAAATGAAGTGATAAAACATTCCTAGAACCTCGAAAAA |
| | AATTCTGCCACTTACAGTTTATGATATTCTTAGATTGGACGGCAAAGATTTCT |
| | CATTTTTTCTCCTTTCTATGAATGGGATAAAACCAAAATATAATAATTTATAA |
| | GCACTAGAAAGTTTGACTTTAGTTCGATTGAGAATAGCACGCTTAGTTTAATT |
| | TCAAAAAAGAATTTGAAGGTTCTTTTTTGTTTCGTAGTAGTAGAATTTCTATA |
| | AAATATAAGATATAAGATTTCGTTGTAGAATCCTAAAATAAAGTAAGTACTT |
| | TTTTAAGTACATGCCAATCTAGTTCTGGACCTCCACATAGCAAAGTCGAAGA |
| | GCTCAACCCAATGTGAGTTTTTTCTATTTTGACTTACTCCCCCCGCCACGATC |
| | GAACGGGAATGGATAAGAGGCTTGTGGGATTGACGTGATAGGGTAGGGTTG |
| | GCTATACTGCTGGTGGTAATACGTGTGCTTGGGAGTCCTTGCAATTTGAATAAAC |
| | CAAGATCTTACCATGAAGACCGGCCGTAAGAACCATCAACGACATCATGTC |
| | CTTCCCGCACGAGGCCGAGTGGGGGCGGCGGCGGTCAGGTGCTCGGCG |
| | GTGTCCCCAGTCACCCCACCATCCCCAGCGCCACCAGCCACTCCACTCC |
| | GACCATGGGGGCCAGCCGAGCCCCGTAAGGGCGCGGACATCCTCGTGG |
| | AGGCGCTGGAGCGATGCGGCGTCAGCGACGTGTTCGCCTATCCAGGCG |
| | GCGCGTCCATGGAGATCCATCAAGCGCTGACTCGTTCCCCAGTCATCAC |
| | CAACCATCTCTTCCGTCATGAGCAAGGCGAGGCGTTCGCGGCGTCCGGG |
| | TATGCGCGTGCGTCCGGCCGTGTCGGGGTCTGCGTCGCCACCTCCGGCC |
| | CCGGGGCAACCAACCTCGTGTCCGCGCTCGCCGACGCGCTGCTCGACTC |
| | CGTCCCAATGGTCGCCATCACTGGCCAAGTCCCCCGTCGTATGATCGGC |
| | ACCGACGCCTTCCAAGAGACTCCCATAGTCGAGGTCACCCGTTCCATCA |
| | CCAAGCATAATTATCTTGTCCTTGATGTGGAGGACATCCCCCGTGTCATA |
| | CAAGAAGCCTTCTTCCTCGCGTCCTCGGGCCGTCCTGGCCCAGTGCTGG |
| | TCGACATCCCCAAGGACATCCAACAACAAATGGCCGTGCCAGTCTGGGA |
| | CACCTCGATGAATCTACCAGGGTATATCGCACGTCTGCCCAAGCCACCC |
| | GCGACAGAATTGCTTGAGCAAGTCTTGCGTCTGGTTGGCGAGTCACGAC |
| | GTCCAATTCTCTATGTCGGTGGTGGCTGCTCTGCATCTGGTGACGAATT |
| | GCGTTGGTTTGTTGAGCTGACTGGTATCCCAGTTACAACCACTCTGATG |
| | GGCCTCGGCAATTTCCCCAGTGACGACCCATTGTCCCTGCGTATGCTTG |
| | GGATGCATGGCACTGTGTATGCAAATTATGCCGTGGATAAGGCTGACCT |
| | GTTGCTTGCGTTTGGTGTGCGATTTGATGATCGTGTGACAGGGAAAATT |
| | GAGGCTTTTGCAAGCAGGGCCAAGATTGTGCATATTGACATTGATCCAG |
| | CAGAGATTGGAAAGAACAAGCAACCACATGTGTCAATTTGCGCAGATGT |
| | TAAACTTGCTTTACAAGGCTTGAATGCTCTGCTACAACAAAGCACAACAA |
| | AGACAAGTTCTGATTTTAGTGCATGGCATAATGAGTTGGACCAACAAAA |
| | GAGGGAGTTTCCTCTGGGGTATAAAACTTTTGGTGAAGAGATCCCACCA |
| | CAATATGCCATTCAAGTGCTGGATGAGCTGACTAAAGGTGAGGCAATCA |
| | TCGCTACTGGTGTTGGGCAACATCAAATGTGGGCGGCACAATATTATAC |
| | CTATAAGCGACCACGACAATGGCTGTCTTCGGCTGGTCTGGGCGCAATG |
| | GGATTTGGGCTGCCTGCTGCAGCTGGTGCTTCTGTGGCTAACCCAGGTG |
| | TCACAGTTGTTGATATTGATGGGGATGGTAGCTTCCTCATGAACATTCAA |
| | GAGCTGGCATTGATCCGTATTGAGAACCTCCCTGTGAAGGTGATGGTGT |
| | TGAACAACCAACATTTGGGTATGGTGGTGCAATTGGAGGATAGGTTTTA |
| | TAAGGCGAATAGGGCGCATACATATTTGGGCAACCCAGAATGTGAGAGC |
| | GAGATATATCCAGATTTTGTGACTATTGCTAAGGGGTTCAATATTCCTGC |
| | AGTCCGTGTAACAAAGAAGAGTGAAGTCCGTGCCGCCATCAAGAAGATG |
| | CTTGAGACTCCAGGGCCATATTTGTTGGATATCATCGTCCCACATCAAG |
| | AGCATGTGCTGCCTATGATCCCAATTGGGGGCGCATTCAAGGACATGAT |
| | CCTGGATGGTGATGGCAGGACTGTGTATTAAGGTACCTTTTTCTCTAAGCT |
| | TGGCTTTTCTGCTAACATATAGCAATTTTTGAAGAAAGGAAAGCTAGAAATACCCAA |
| | TATCTTGCTGAAGCAAGATATTGGGTATTTCTTTTTTTTTTATTTTGAATCTTTCTATT |
| | CTGAATTATCCAAAAAAAGCATGAATAGATTTTTTGAAAAAGATTGGAAAAA |
| | AGTAAGTAGAATTAGATTTAAGATAAAAGAAAGTTAGTTTTAGTAAGAAACG |
| | TGGATGAATACTTGCTCTTTTCTCGATTTTAGATCGTATTTTTTGGCGGCATG |
| | GCCAAGCGGTAAGGCAGGGGACTGCAAATCCTTTATCCCCAGTTCAAATCTG |
| | GGTGCCGCCTGATCAATAAAATACTTAGGCTTATAGTACTGATCCAAATCGA |
| | CAAATTTGCACCCCGCATAAGTTGGGGCAAGAGAATAACAAGGCTCGGCGA |
| | TACTGGATTTTGAGAACCCACCGTTCTTTCCTCAAACTGGGGTTTGTTTTAGC |
| | AGTAGTGGCCCAAACCGATACCAATAGGGATGAGATAGCGTTTCCTTATTCT |
| | TTTATTAATAAGAAAGGGCTATGTATCATATTTATACTTAATGCTCCCCAATT |
| | CTACCAGAAATACTCTAAGAATTTGTTAAGAGTAAATTCTTTGAACTGATTCA |
| | TCCATAGCCTTAGGTCAGAACCCCTTTTTGACTCTGTACCCTTGATTCTACTA |
| | TGATTATTCATCAATAGTAGAATAATTCCTTCATAGAAATAGGAGACATAAT |
| | TTACATGGATATAGTAAGTCTCGCTTGGGCTGCTTTAATGGTAGTCTTTACAT |
| | TTTCTCTTTCACTAGTAGTATGGGGGAGGAGTGGACTCTAGGGATACTACTA |
| | ATTGATTGAGTAGTCGAATTGTTGTATCAACTCTTTTCTTTAATTGACCGTTTT |
| | GCATTAATCATTTTGCCGAACTTTATTCTTTCTCTCTTTCTTTAATTTTGTTTCA |
| | CTCCTGTAAGAAACTCTTGTAAGAAAGAGTCTTTCTATTCTTTCTATTTCTATT |
| | TCTATATAGTAGAATAGAAATAGAAAGAATAGAAAATAGAAATAGAAAGAG |
| | CGGTGAGAGCAATTCATAATCGCTACTAGAAGCGACCCATGGTTAAAAAAGT |
| | TCTATACATAAGAACTTTAGATTTTCTTCCGCGGAGCTATTCCCAACATATCG |
| | CACATTTTCCATTTGTTTTATATTCTTTTCTTATTCTTAGTTCTTAAAATAAATT |
| | TTATGCTCTTTTGAAACATTTCGCGACATGTTTAAGCATGAAAGATTCGCTGG |
| | TTTTTTCCTTTTACTTTTTTTCTTTTACTATAGTCTATTCTCATATTATTTTTCTC |
| | TCCCTCCATGGATTTTTTCATTTTGACTTGATTGAAATTAAGTGGATGCAGTG |
| | AAAAAAGAAATTGAATTAGACTACTATTTCAATCTAGTAATCTATTCTATGTA |
| | GATTCAAGATAATATAATAGAAAGACTCTAAAGTGTGGTAGAAAAAACTAT |
| | ATGTAGTTTTTTCTACCACACTTTATGATTCCAACCGGATCCTTTCATTTAAG |
| | ACTTGGATTTTTATTTTATTTAATCCCTTTTTCATTTCTTCAATGATTAATTGA |
| | ACTTCCAATTAATCATTTTGGCTGACTGTTTTTACATAAATAATAAGTAAAAA |
| | GGCAGTAGGAACTAGAATGAACAATGCAGTAGCAATAAATGCGAGAATATT |
| | GACTTCCATAACCTCTTTTTTTTCGCAATAACTCGGGATGTAATCCCATGGAG |
| | AGGGAAAAGGGTATCCTGTAAATTCAAGGGGAGGACTTGCATTCTGATAATA |
| | CTGAATCAATTCAATATTATGAATATCGGATCTATCAAATCAATTCATGGTTG |
| | AGAGTGGAATAGTATAACATAGGAGGATCCCTTCCACGTATGATATGTATGT |
| | CTTTCGCTATCAACCAGAAGTAGTGATAAAAAAATTCCTATACTCTCCTCTCT |
| | TTACTGAGAAACGCGAACTAAAAAAAGTGAAATAGGGGTACCCCATAGGTA |
| | TTTGATCTTGCTTCCTGCCCCCCTTTTTTCATGGAAAAAGGAAAGATGAATTT |
| | CTCCATTCATTGAACCCTAGTT |
| 70 | AACCCAATGTGAGTTTTTTCTATTTTGACTTACTCCCCCCGCCACGATCGAAC |
| | GGGAATGGATAAGAGGCTTGTGGGATTGACGTGATAGGGTAGGGTTGGCTAT |
| | ACTGCTGGTGG |
| 71 | TAATACGTGTGCTTGGGAGTCCTTGCAATTTGAATAAACCAAGATCTTACC |
| 72 | GGCTTTTCTGCTAACATATAGCAATTTTTGAAGAAAGGAAAGCTAGAAATAC |
| | CCAATATCTTGCTGAAGCAAGATATTGGGTATTTCTTTTTTTTTTATTTTGAAT |
| | CTTTCTATTCTGAATT |
| 73 | CCACCTGTCTCTCTTTTTTTTTCTTTGCTTTCTCAGATATTTCATAAAAAGGAC |
| | TCTCTACGGATCCCCACCAATGATCAATTCTCGCATGAATAGCTAAAGATCC |
| | AGTAAGTCCAACGTTGATTCCTTCGGACGTGTCAATTGGACAAATACGCCCA |
| | TAGTGACTCGGATGGATATCTCGGCTCCGAAAACTTGCAGTTCTCCCCGTCA |
| | ATCCTCCAGGACCCAAACAACTCACTTTTCGCCCATGAACCGTTTGTGTCAAT |
| | GGATTGGTTTGATCAAAAACTTGAGATAAGGGATATGTACCAAAGAAGGTCT |
| | CGTAAGTAGTTATTAATAAAATCGAGGTTGAAGTTGGAGTTACCAAAGTTTG |
| | TGGAGTCGGTTTTGATTGACGTATGAATACTCTACGGATAGTTTTTTGAACCG |
| | CATGTTGTAAACGACCAAGAGCCAGTCCGAATTGATCTTGTAACAGATCCGC |
| | AACCGAACGAATACGTTTATTTTTCAAGTGATTCATATCGTCATCGTCAAGTA |
| | TACCCGTTTCAAATTTCATTCCAATCAAATGATCCGTAGCGGCCAATACATCT |
| | CGCGGTAACAAGAAGGTGCTGTTCTGAGGTATATCAAGATTCAGTCTTCGAT |
| | TCATATTTCGTCGACCAATCCTTCCTAATTCACATTTTTGTTGAAAAAATTTCT |
| | TTTGTAATTCCTCACATAAGGATTCCGAAAATACCAGGTCCCCACCTACACA |
| | AGCAAATTGTTGATAAAACTCCAAAATAGCTTTTTCTTTTGACTCAATCCTCT |
| | TCTTCTCCTTAGCATTAGGGAAAGACAAGAAAATTTCAGGGTAGGAAACATT |
| | ATCTAGAATTTCTTTTAGATTTGAACCCATAGCTGATGATAGAACTAGAATA |
| | GATATCTTTTGTTTTCTACTCACGCGAGCCCATATCCTTTCTTTTTTATCAATT |
| | GCTAATTCCGATCTTCCTCCCCAATCTGATATTATAGTCCCGGTGTAGATAGA |
| | AATTCCTTTATGGTCTAATTCCGAGCGGTAGTAAATACCAGGACTTAGCAAT |
| | ATTTGATTGATCACAATTCGGTATATTCCATTTATTATAAAGGTTCCTAAGGA |
| | ATTCATTATAGGAATGTTTCCAATAGAAATGGTTTGCTTTTGCACATCGAAAC |
| | CAAAAATTAATCGCGCGGATACGTATAATTCGGAAGAATAGGTGAGTGATTC |
| | ATACACAGCATCCCTTTCTTTTATCGAAGGTTCTAGCAATTGATATCCTTTCG |
| | CAAATAATTGAAATGAAATTTCGTGATCTGGATCTTTAATTGTTGGAAACTTC |
| | TCAAGTTCTTCTGCCAAGCCTTGATTAATGAACCTACAAAATCCCTCGAATTG |
| | GATCTGACTAAATCCGGGTATTGTGGACATTCCCTCATTTCCATTCCGGAGCA |
| | TCTTAATCTTAAGTTTCCTGTTTATCGAAGAAAAATTCCATTATCAGCTCACT |
| | CTTCATCAATCCCTACAGATCGATCTAGCAATTATGGAATCTATATTCTGTTT |
| | ACTAAATCACATGAAATTCTCGGGAACTCCACATACGTATTTCATATATGTAT |
| | TTCATACATATGAATAGAGACAATTTCAACGAAAGTTCGAATTTCCCAGGGG |
| | TCCAAATGGAATGAAAATGAAGTGATAAAACATTCCTAGAACCTCGAAAAA |
| | AATTCTGCCACTTACAGTTTATGATATTCTTAGATTGGACGGCAAAGATTTCT |
| | CATTTTTTCTCCTTTCTATGAATGGGATAAAACCAAAATATAATAATTTATAA |
| | GCACTAGAAAGTTTGACTTTAGTTCGATTGAGAATAGCACGCTTAGTTTAATT |
| | TCAAAAAAGAATTTGAAGGTTCTTTTTTGTTTCGTAGTAGTAGAATTTCTATA |
| | AAATATAAGATATAAGATTTCGTTGTAGAATCCTAAAATAAAGTAAGTACTT |
| | TTTTAAGTACATGCCAATCTAGTTCTGGACCTCCACATAGCAAAGTCGAA |
| 74 | ATCCAAAAAAAGCATGAATAGATTTTTTGAAAAAGATTGGAAAAAAGTAAG |
| | TAGAATTAGATTTAAGATAAAAGAAAGTTAGTTTTAGTAAGAAACGTGGATG |
| | AATACTTGCTCTTTTCTCGATTTTAGATCGTATTTTTTGGCGGCATGGCCAAG |
| | CGGTAAGGCAGGGGACTGCAAATCCTTTATCCCCAGTTCAAATCTGGGTGCC |
| | GCCTGATCAATAAAATACTTAGGCTTATAGTACTGATCCAAATCGACAAATT |
| | TGCACCCCGCATAAGTTGGGGCAAGAGAATAACAAGGCTCGGCGATACTGG |
| | ATTTTGAGAACCCACCGTTCTTTCCTCAAACTGGGGTTTGTTTTAGCAGTAGT |
| | GGCCCAAACCGATACCAATAGGGATGAGATAGCGTTTCCTTATTCTTTTATTA |
| | ATAAGAAAGGGCTATGTATCATATTTATACTTAATGCTCCCCAATTCTACCAG |
| | AAATACTCTAAGAATTTGTTAAGAGTAAATTCTTTGAACTGATTCATCCATAG |
| | CCTTAGGTCAGAACCCCTTTTTGACTCTGTACCCTTGATTCTACTATGATTATT |
| | CATCAATAGTAGAATAATTCCTTCATAGAAATAGGAGACATAATTTACATGG |
| | ATATAGTAAGTCTCGCTTGGGCTGCTTTAATGGTAGTCTTTACATTTTCTCTTT |
| | CACTAGTAGTATGGGGGAGGAGTGGACTCTAGGGATACTACTAATTGATTGA |
| | GTAGTCGAATTGTTGTATCAACTCTTTTCTTTAATTGACCGTTTTGCATTAATC |
| | ATTTTGCCGAACTTTATTCTTTCTCTCTTTCTTTAATTTTGTTTCACTCCTGTAA |
| | GAAACTCTTGTAAGAAAGAGTCTTTCTATTCTTTCTATTTCTATTTCTATATAG |
| | TAGAATAGAAATAGAAAGAATAGAAAATAGAAATAGAAAGAGCGGTGAGA |
| | GCAATTCATAATCGCTACTAGAAGCGACCCATGGTTAAAAAAGTTCTATACA |
| | TAAGAACTTTAGATTTTCTTCCGCGGAGCTATTCCCAACATATCGCACATTTT |
| | CCATTTGTTTTATATTCTTTTCTTATTCTTAGTTCTTAAAATAAATTTTATGCTC |
| | TTTTGAAACATTTCGCGACATGTTTAAGCATGAAAGATTCGCTGGTTTTTTCC |
| | TTTTACTTTTTTTCTTTTACTATAGTCTATTCTCATATTATTTTTCTCTCCCTCC |
| | ATGGATTTTTTCATTTTGACTTGATTGAAATTAAGTGGATGCAGTGAAAAAA |
| | GAAATTGAATTAGACTACTATTTCAATCTAGTAATCTATTCTATGTAGATTCA |
| | AGATAATATAATAGAAAGACTCTAAAGTGTGGTAGAAAAAACTATATGTAGT |
| | TTTTTCTACCACACTTTATGATTCCAACCGGATCCTTTCATTTAAGACTTGGAT |
| | TTTTATTTTATTTAATCCCTTTTTCATTTCTTCAATGATTAATTGAACTTCCAA |
| | TTAATCATTTTGGCTGACTGTTTTTACATAAATAATAAGTAAAAAGGCAGTA |
| | GGAACTAGAATGAACAATGCAGTAGCAATAAATGCGAGAATATTGACTTCC |
| | ATAACCTCTTTTTTTTCGCAATAACTCGGGATGTAATCCCATGGAGAGGGAA |
| | AAGGGTATCCTGTAAATTCAAGGGGAGGACTTGCATTCTGATAATACTGAAT |
| | CAATTCAATATTATGAATATCGGATCTATCAAATCAATTCATGGTTGAGAGT |
| | GGAATAGTATAACATAGGAGGATCCCTTCCACGTATGATATGTATGTCTTTC |
| | GCTATCAACCAGAAGTAGTGATAAAAAAATTCCTATACTCTCCTCTCTTTACT |
| | GAGAAACGCGAACTAAAAAAAGTGAAATAGGGGTACCCCATAGGTATTTGA |
| | TCTTGCTTCCTGCCCCCCTTTTTTCATGGAAAAAGGAAAGATGAATTTCTCCA |
| | TTCATTGAACCCTAGTT |
| 75 | ATGGTGGAGCACGACACTCTCGTCTACTCCAAGAATATCAAAGATACAGTCT |
| | CAGAAGACCAAAGGGCTATTGAGACTTTTCAACAAAGGGTAATATCGGGAA |
| | ACCTCCTCGGATTCCATTGCCCAGCTATCTGTCACTTCATCAAAAGGACAGTA |
| | GAAAAGGAAGGTGGCACCTACAAATGCCATCATTGCGATAAAGGAAAGGCT |
| | ATCGTTCAAGATGCCTCTGCCGACAGTGGTCCCAAAGATGGACCCCCACCCA |
| | CGAGGAGCATCGTGGAAAAAGAAGACGTTCCAACCACGTCTTCAAAGCAAG |
| | TGGATTGATGTGATAACATGGTGGAGCACGACACTCTCGTCTACTCCAAGAA |
| | TATCAAAGATACAGTCTCAGAAGACCAAAGGGCTATTGAGACTTTTCAACAA |
| | AGGGTAATATCGGGAAACCTCCTCGGATTCCATTGCCCAGCTATCTGTCACTT |
| | CATCAAAAGGACAGTAGAAAAGGAAGGTGGCACCTACAAATGCCATCATTG |
| | CGATAAAGGAAAGGCTATCGTTCAAGATGCCTCTGCCGACAGTGGTCCCAAA |
| | GATGGACCCCCACCCACGAGGAGCATCGTGGAAAAAGAAGACGTTCCAACC |
| | ACGTCTTCAAAGCAAGTGGATTGATGTGATATCTCCACTGACGTAAGGGATG |
| | ACGCACAATCCCACTATCCTTCGCAAGACCTTCCTCTATATAAGGAAGTTCAT |
| | TTCATTTGGAGAGGACACGCTGAAATCACCAGTCTCTCTCTACAAATCTATCT |
| | CTCTCGAGCTTTCGCAGATCCCGGGGGGCAATGAGATATGAAAAAGCCTGAA |
| | CTCACCGCGACGTCTGTCGAGAAGTTTCTGATCGAAAAGTTCGACAGCGTCT |
| | CCGACCTGATGCAGCTCTCGGAGGGCGAAGAATCTCGTGCTTTCAGCTTCGA |
| | TGTAGGAGGGCGTGGATATGTCCTGCGGGTAAATAGCTGCGCCGATGGTTTC |
| | TACAAAGATCGTTATGTTTATCGGCACTTTGCATCGGCCGCGCTCCCGATTCC |
| | GGAAGTGCTTGACATTGGGGAGTTTAGCGAGAGCCTGACCTATTGCATCTCC |
| | CGCCGTGCACAGGGTGTCACGTTGCAAGACCTGCCTGAAACCGAACTGCCCG |
| | CTGTTCTACAACCGGTCGCGGAGGCTATGGATGCGATCGCTGCGGCCGATCT |
| | TAGCCAGACGAGCGGGTTCGGCCCATTCGGACCGCAAGGAATCGGTCAATAC |
| | ACTACATGGCGTGATTTCATATGCGCGATTGCTGATCCCCATGTGTATCACTG |
| | GCAAACTGTGATGGACGACACCGTCAGTGCGTCCGTCGCGCAGGCTCTCGAT |
| | GAGCTGATGCTTTGGGCCGAGGACTGCCCCGAAGTCCGGCACCTCGTGCACG |
| | CGGATTTCGGCTCCAACAATGTCCTGACGGACAATGGCCGCATAACAGCGGT |
| | CATTGACTGGAGCGAGGCGATGTTCGGGGATTCCCAATACGAGGTCGCCAAC |
| | ATCTTCTTCTGGAGGCCGTGGTTGGCTTGTATGGAGCAGCAGACGCGCTACTT |
| | CGAGCGGAGGCATCCGGAGCTTGCAGGATCGCCACGACTCCGGGCGTATATG |
| | CTCCGCATTGGTCTTGACCAACTCTATCAGAGCTTGGTTGACGGCAATTTCGA |
| | TGATGCAGCTTGGGCGCAGGGTCGATGCGACGCAATCGTCCGATCCGGAGCC |
| | GGGACTGTCGGGCGTACACAAATCGCCCGCAGAAGCGCGGCCGTCTGGACC |
| | GATGGCTGTGTAGAAGTACTCGCCGATAGTGGAAACCGACGCCCCAGCACTC |
| | GTCCGAGGGCAAAGAAATAGAGTAGATGCCGACCGGATCTGTCGATCGACA |
| | AGCTCGAGTTTCTCCATAATAATGTGTGAGTAGTTCCCAGATAAGGGAATTA |
| | GGGTTCCTATAGGGTTTCGCTCATGTGTTGAGCATATAAGAAACCCTTAGTAT |
| | GTATTTGTATTTGTAAAATACTTCTATCAATAAAATTTCTAATTCCTAAAACC |
| | AAAATCCAGTACTAAAATCCAGATCCCCCGAATTA |
| 76 | CTAACATGAATCTGTTCCCATGCAATAGTC |
| 77 | ACGTCAATCCCACAAGCCTCTTATCC |
| 78 | GCGGGGATTGTAGTTCAATTGGTCAG |
| 79 | GATGGCAGGACTGTGTATTAAggtacc |
| 80 | TTCCTGAATACCCCGATTCAAGGACAAAGAATTTCCTGCTGCTATCATATAAT |
| | ACTCATCTCTATTTGGTGATAAATAAACCACCTGTCTCTCTTTTTTTTTCTTTG |
| | CTTTCTCAGATATTTCATAAAAAGGACTCTCTACGGATCCCCACCAATGATCA |
| 81 | TTGTAGAATCCTAAAATAAAGTAAGTACTTTTTTAAGTACATGCCAATCTAGT |
| | TCTGGACCTCCACATAGCAAAGTCGAAGAGCTCAACCCAATGTGAGTTTTTT |
| | CTATTTTGACTTACTCCCCCCGCC |
| 82 | CTAGAAATACCCAATATCTTGCTGAAGCAAGATATTGGGTATTTCTTTTTTTT |
| | TTATTTTGAATCTTTCTATTCTGAATTATCCAAAAAAAGCATGAATAGATTTT |
| | TTGAAAAAGATTGGAAAAAAGTAAGTA |
| 83 | TAGGTATTTGATCTTGCTTCCTGCCCCCCTTTTTTCATGGAAAAAGGAAAGAT |
| | GAATTTCTCCATTCATTGAACCCTAGTTCGGGACTGACGGGGCTCGAACCCG |
| | CAGCT |
| 84 | CCGAACAGCATTATTATC |
| 85 | CTCCACAGAGATATGTAC |
| 86 | ATGGCCCCCTCCGTGATGGCGTCGTCGGCCACCACCGTCGCTCCCTTCCAGG |
| | GCTCAAGTCCACCGCCGGCTTGCCGTCGCCCGCCGTCCGAACTCCAGCTTCG |
| | GCAACGTCAGCATGGCGGCAGGATCAGATGCACCGGCCGCGCCACCATGGG |
| | AAAACCTATTCCTAATCCTCTGCTGGGCCTGGATTCTACCGGAGGCGTGGAC |
| | CTGAGAACACTGGGATATTCTCAGCAGCAGCAGGAGAAGATCAAGCCCAAG |
| | GTGAGATCTACAGTGGCCCAGCACCACGAAGCCCTGGTGGGACACGGATTTA |
| | CACACGCCCACATTGTGGCCCTGTCTCAGCACCCTGCCGCCCTGGGAACAGT |
| | GGCCGTGAAATATCAGGATATGATTGCCGCCCTGCCTGAGGCCACACACGAA |
| | GCCATTGTGGGAGTGGGAAAACGAGGCGCTGGAGCCAGAGCCCTGGAAGCC |
| | CTGCTGACAGTGGCCGGAGAACTGAGAGGACCTCCTCTGCAGCTGGATACAG |
| | GACAGCTGCTGAAGATTGCCAAAAGGGGCGGAGTGACCGCGGTGGAAGCCG |
| | TGCACGCCTGGAGAAATGCCCTGACAGGAGCCCCTCTGAACCTGACCCCCGA |
| | ACAGGTGGTGGCCATTGCCAGCCACGACGGCGGCAAGCAGGCCCTGGAAAC |
| | CGTGCAGAGACTGCTGCCCGTGCTGTGCCAGGCCCATGGCCTGACACCTGAA |
| | CAGGTGGTGGCTATCGCCTCTCACGACGGAGGAAAACAGGCTCTGGAAACA |
| | GTGCAGCGGCTGCTGCCTGTGCTGTGTCAGGCTCACGGCTTGACTCCAGAAC |
| | AGGTGGTGGCTATTGCTTCCAACAACGGGGGGAAACAGGCCCTGGAAACTGT |
| | GCAGCGCCTGCTGCCAGTGCTGTGCCAGGCTCACGGACTGACCCCCGAACAG |
| | GTGGTGGCCATTGCCAGCAACATCGGCGGCAAGCAGGCCCTGGAAACCGTG |
| | CAGAGACTGCTGCCCGTGCTGTGCCAGGCCCATGGCCTGACACCTGAACAGG |
| | TGGTGGCTATCGCCTCTAATATCGGAGGAAAACAGGCTCTGGAAACAGTGCA |
| | GCGGCTGCTGCCTGTGCTGTGTCANGCTCACGGCTTGACTCCAGAACAGGTG |
| | GTGGCTATTGCTTCCCACGACGGGGGGAAACAGGCCCTGGGAACTGTGCAGC |
| | GCCTGCTGCCAGTGCTGTGCCAGGCTCACGGGCTGACCCCCGAACAGGTGGT |
| | GGCCATTGCCAGCAACATCGGCGGCAAGCAGGCCCTGGAAACCGTGCAGAG |
| | ACTGCTGCCCGTGCTGTGCCAGGCCCATGGCCTGACACCTGAACAGGTGGTG |
| | GCTATCGCCTCTAACAACGGANGAAAACNGCTCTGGAAACAGTGCAGCGGC |
| | TGCTGCCTGTGCTGTGTCNNNTCACGGCTTTTGACTCCANAACAGGTGGTGG |
| | CTATTGCTTCCCACNACGGGGGGAAACAGGCCCTGGAAACTGTGCAGCNCCT |
| | GCTGCCAGTGCTGTGCCANGCTCACGGGCTGACCCCCGAACAGGTGGTGGCC |
| | ATTGCCAGCAACATCGGCGGCAAGCAGGCCCTGGAAACCGTGCAGAGACTG |
| | CTGCCCGTGCTGTGCCAGGCCCATGGCCTGACACCTGAACAGGTGGTGGCTA |
| | TCGCCTCTAACGGCGGAGGAAAACAGGCTCTGGAAACAGTGCAGCGGCTGC |
| | TGCCTGTGCTGTGTCAGGCTCACGGCTTGACTCCAGAACAGGTGGTGGCTAT |
| | TGCTTCCAACGGCGGGGGGAAACAGGCCCTGGAAACTGTGCAGCGCCTGCTG |
| | CCAGTGCTGTGCCAGGCTCACGGACTGACCCCCGAACAGGTGGTGGCCATTG |
| | CCAGCAACATCGGCGGCAAGCAGGCCCTGGAAACCGTGCAGAGACTGCTGC |
| | CCGTGCTGTGCCAGGCCCATGGCCTGACACCTGAACAGGTGGTGGCTATCGC |
| | CTCTAACGGCGGAGGAAAACAAGCACTCGAGACAGTGCAGCGGCTGCTGCC |
| | TGTGCTGTGTCAGGCTCACGGCTTGACTCCAGAACAGGTGGTGGCTATTGCTT |
| | CCAACGGCGGGGGGAAACAGGCCCTGGAAACTGTGCAGCGCCTGCTGCCAG |
| | TGCTGTGCCAGGCTCACGGCCTGACCCCCGAACAGGTGGTGGCCATTGCCAG |
| | CAACATCGGCGGCAAGCAGGCCCTGGAAACCGTGCAGAGACTGCTGCCCGT |
| | GCTGTGCCAGGCCCATGGCCTGACACCTGAACAGGTGGTGGCTATCGCCTCT |
| | AACGGCGGAGGAAAACAGGCTCTGGAAACAGTGCAGCGGCTGCTGCCTGTG |
| | CTGTGTCAGGCTCACGGCTTGACTCCACAGCAGGTCGTGGCAATTGCTAGCC |
| | ACGACGGCGGACGGCCCGCCCTGGAGAGCATTGTGGCCCAGCTGTCTAGACC |
| | GGACCCAGCTCTCGCTGCATTGACTAACGACCATCTCGTCGCTTTGGCTTGCC |
| | TCGGTGGAAGGCCAGCTCTTGACGCTGTAAAGAAAGGTTTGCCACATGCTCC |
| | AGCTCTTATTAAAAGGACTGGTAGCGGTAGCATGAATGAATTAACCTTGCAC |
| | CCTAACGTCTACGCAACAGGAGCCTCCAAGGGTTTGATACACGTATTGGAAA |
| | GGGTGTGGGTAAGGGAGCATACTCCAGGTGATGGAACAATATTTGTCATTAG |
| | CGGATTCGGAAATTATAATGGAGGTGTAAGGTTCTTTGAATCCTTCAGGGAG |
| | CATAAACGCAGGGGAGGTGAATTGGTTGCTGTCTTTGCAGGATCAACCTCGG |
| | CTAGGTTGACTTCTAAACAAGTAGTCCATGAAATGTTGGACTGTGGTGCAGA |
| | CGTACATATTGTAAATCGCAAAAGGTTGCTTCATGCAAAATGCTATGGTACG |
| | TCCAAAATGAGCGGAGACAGCTTGATAGTCACAAGCGGTAACTTTACAGGGC |
| | CTGGTATGAGCCAAAATGTCGAGATGAGCGTGCTCTTGGATGCTGACACGAC |
| | TGCGGAAATGGGATTTAGCTGGGGTACACTCTTGGAAAATCTGTTGGACCAA |
| | AGGTGGAATTATCACAGACCGACGCTCGGCGACCCTGCGAACCCTGCATGGT |
| | CGTTGCTGTACGACGAACAAGCTGCGGACATACGCCTCGACGAGACAGAGG |
| | AATGA |
| 87 | MAPSVMASSATTVAPFQGSSPPPACRRPPSELQLRQRQHGGRIRCTGRATMGKPI |
| | PNPLLGLDSTGGVDLRTLGYSQQQQEKIKPKVRSTVAQHHEALVGHGFTHAHIV |
| | ALSQHPAALGTVAVKYQDMIAALPEATHEAIVGVGKRGAGARALEALLTVAGE |
| | LRGPPLQLDTGQLLKIAKRGGVTAVEAVHAWRNALTGAPLNLTPEQVVAIASH |
| | DGGKQALETVQRLLPVLCQAHGLTPEQVVAIASHDGGKQALETVQRLLPVLCQ |
| | AHGLTPEQVVAIASNNGGKQALETVQRLLPVLCQAHGLTPEQVVAIASNIGGKQ |
| | ALETVQRLLPVLCQAHGLTPEQVVAIASNIGGKQALETVQRLLPVLCXAHGLTP |
| | EQVVAIASHDGGKQALGTVQRLLPVLCQAHGLTPEQVVAIASNIGGKQALETVQ |
| | RLLPVLCQAHGLTPEQVVAIASNNGXKXLWKQCSGCCLCCVXSRLLTPXQVVAI |
| | ASHXGGKQALETVQXLLPVLCXAHGLTPEQVVAIASNIGGKQALETVQRLLPVL |
| | CQAHGLTPEQVVAIASNGGGKQALETVQRLLPVLCQAHGLTPEQVVAIASNGG |
| | GKQALETVQRLLPVLCQAHGLTPEQVVAIASNIGGKQALETVQRLLPVLCQAHG |
| | LTPEQVVAIASNGGGKQALETVQRLLPVLCQAHGLTPEQVVAIASNGGGKQALE |
| | TVQRLLPVLCQAHGLTPEQVVAIASNIGGKQALETVQRLLPVLCQAHGLTPEQV |
| | VAIASNGGGKQALETVQRLLPVLCQAHGLTPQQVVAIAXHDGXXPALXXIXXLL |
| | SIPDPALASRPDPALAALTNDHLVALACLGGRPALDAVKKGLPHAPALIKRTGSG |
| | SMNELTLHPNVYATGASKGLIHVLERVWVREHTPGDGTIFVISGFGNYNGGVRF |
| | FESFREHKRRGGELVAVFAGSTSARLTSKQVVHEMLDCGADVHIVNRKRLLHA |
| | KCYGTSKMSGDSLIVTSGNFTGPGMSQNVEMSVLLDADTTAEMGFSWGTLLEN |
| | LLDQRWNYHRPTLGDPANPAWSLLYDEQAADIRLDETEE |
| 88 | MAPSVMASSATTVAPFQGSSPPPACRRPPSELQLRQRQHGGRIRC |
| 89 | ATGGCTCCCAGCGTCATGGCCTCCAGCGCGACAACTGTGGCCCCGTTTCAGG |
| | GTTCGAGCCCGCCACCTGCTTGCCGCCGGCCACCTAGCGAGCTGCAACTCCG |
| | CCAGCGCCAACACGGAGGGAGAATTAGATGCACCGGCCGCGCCACCATGGG |
| | AAAACCGATCCCAAACCCGCTTTTGGGGCTCGACAGCACAGGAGGGGTTGAT |
| | CTCAGGACCCTCGGTTACAGCCAACAGCAACAAGAAAAAATTAAGCCTAAA |
| | GTGAGGTCCACGGTAGCTCAACATCACGAGGCTTTGGTAGGGCATGGCTTCA |
| | CGCACGCTCATATCGTCGCTCTCAGCCAACATCCCGCTGCGCTTGGGACGGT |
| | CGCTGTTAAGTACCAAGACATGATCGCTGCGCTTCCGGAAGCTACGCATGAG |
| | GCGATCGTCGGTGTAGGGAAGCGGGGAGCAGGTGCGAGGGCGCTCGAGGCG |
| | CTCTTGACCGTCGCGGGTGAGCTTAGGGGTCCGCCACTCCAATTGGACACCG |
| | GACAACTTTTGAAAATCGCTAAGAGAGGAGGCGTCACAGCCGTCGAGGCGG |
| | TGCACGCCTGGAGAAATGCCCTGACAGGAGCCCCTCTGAACCTGACCCCCGA |
| | ACAGGTGGTGGCCATTGCCAGCCACGACGGCGGCAAGCAGGCCCTGGAAAC |
| | CGTGCAGAGACTGCTGCCCGTGCTGTGCCAGGCCCATGGCCTGACACCTGAA |
| | CAGGTGGTGGCTATCGCCTCTAACGGCGGAGGAAAACAGGCTCTGGAAACA |
| | GTGCAGCGGCTGCTGCCTGTGCTGTGTCAGGCTCACGGCTTGACTCCAGAAC |
| | AGGTGGTGGCTATTGCTTCCCACGACGGGGGGAAACAGGCCCTGGAAACTGT |
| | GCAGCGCCTGCTGCCAGTGCTGTGCCAGGCTCACGGACTGACCCCCGAACAG |
| | GTGGTGGCCATTGCCAGCCACGACGGCGGCAAGCAGGCCCTGGAAACCGTG |
| | CAGAGACTGCTGCCCGTGCTGTGCCAGGCCCATGGCCTGACACCTGAACAGG |
| | TGGTGGCTATCGCCTCTAATATCGGAGGAAAACAGGCTCTGGAAACAGTGCA |
| | GCGGCTGCTGCCTGTGCTGTGTCAGGCTCACGGCTTGACTCCAGAACAGGTG |
| | GTGGCTATTGCTTCCCACGACGGGGGGAAACAGGCCCTGGAAACTGTGCAGC |
| | GCCTGCTGCCAGTGCTGTGCCAGGCTCACGGGCTGACCCCCGAACAGGTGGT |
| | GGCCATTGCCAGCAACATCGGCGGCAAGCAGGCCCTGGAAACCGTGCAGAG |
| | ACTGCTGCCCGTGCTGTGCCAGGCCCATGGCCTGACACCTGAACAGGTGGTG |
| | GCTATCGCCTCTAACAACGGAGGAAAACAGGCTCTGGAAACAGTGCAGCGG |
| | CTGCTGCCTGTGCTGTGTCAGGCTCACGGCTTGACTCCAGAACAGGTGGTGG |
| | CTATTGCTTCCAATATTGGGGGGAAACAGGCCCTGGAAACTGTGCAGCGCCT |
| | GCTGCCAGTGCTGTGCCAGGCTCACGGCCTCACTCCCGAACAGGTGGTGGCC |
| | ATTGCCAGCAACAACGGCGGCAAGCAGGCCCTGGAAACCGTGCAGAGACTG |
| | CTGCCCGTGCTGTGCCAGGCCCATGGCCTGACACCTGAACAGGTGGTGGCTA |
| | TCGCCTCTAATATCGGAGGAAAACAGGCTCTGGAAACAGTGCAGCGGCTGCT |
| | GCCTGTGCTGTGTCAGGCTCACGGCTTGACTCCAGAACAGGTGGTGGCTATT |
| | GCTTCCAACGGCGGGGGGAAACAGGCCCTGGAAACTGTGCAGCGCCTGCTG |
| | CCAGTGCTGTGCCAGGCTCACGGACTGACCCCCGAACAGGTGGTGGCCATTG |
| | CCAGCAACATCGGCGGCAAGCAGGCCCTGGAAACCGTGCAGAGACTGCTGC |
| | CCGTGCTGTGCCAGGCCCATGGCCTGACACCTGAACAGGTGGTGGCTATCGC |
| | CTCTAACGGCGGAGGAAAACAAGCACTCGAGACAGTGCAGCGGCTGCTGCC |
| | TGTGCTGTGTCAGGCTCACGGCTTGACTCCAGAACAGGTGGTGGCTATTGCTT |
| | CCAACAACGGGGGGAAACAGGCCCTGGAAACTGTGCAGCGCCTGCTGCCAG |
| | TGCTGTGCCAGGCTCACGGCCTGACCCCCGAACAGGTGGTGGCCATTGCCAG |
| | CAACGGCGGCGGCAAGCAGGCCCTGGAAACCGTGCAGAGACTGCTGCCCGT |
| | GCTGTGCCAGGCCCATGGCCTGACACCTGAACAGGTGGTGGCTATCGCCTCT |
| | AATATCGGAGGAAAACAGGCTCTGGAAACAGTGCAGCGGCTGCTGCCTGTG |
| | CTGTGTCAGGCTCACGGCTTGACTCCACAGCAGGTCGTGGCAATTGCTAGCC |
| | ACGACGGCGGACGGCCCGCCCTGGAGAGCATTGTGGCCCAGCTGTCTAGACC |
| | TGATCCTGCCCTGGCCGCCCTGACAAATGATCACCTGGTGGCCCTGGCCTGTC |
| | TGGGAGGCAGACCTGCCCTGGATGCCGTGAAAAAAGGACTGCCTCACGCCCC |
| | TGCCCTGATCAAGAGAACAGGATCCGGATCCATGAACGAGTTAACGCTGCAT |
| | CCGAATGTGTATGCGACCGGCGCGAGTAAAGGGCTGATACATGTGCTGGAGC |
| | GCGTATGGGTTCGAGAACACACGCCTGGGGACGGCACGATCTTCGTGATATC |
| | TGGCTTTGGCAACTACAACGGTGGCGTTCGCTTCTTCGAGTCGTTTCGTGAAC |
| | ACAAGAGACGTGGCGGGGAGCTGGTAGCGGTGTTCGCTGGCAGCACAAGCG |
| | CGCGACTGACAAGCAAACAGGTTGTTCACGAGATGCTTGATTGCGGGGCGGA |
| | TGTTCACATAGTGAACAGAAAGCGACTCCTTCACGCTAAGTGTTACGGCACA |
| | AGCAAGATGTCAGGTGATTCCCTAATTGTAACCTCAGGCAATTTCACAGGTC |
| | CGGGAATGTCGCAGAACGTGGAAATGTCTGTATTGCTAGACGCAGATACAAC |
| | CGCCGAGATGGGGTTCTCGTGGGGCACGTTGCTTGAGAACTTGCTTGATCAG |
| | CGATGGAACTACCATCGCCCCACCTTAGGCGATCCAGCAAATCCAGCCTGGA |
| | GCCTGCTTTATGATGAGCAGGCAGCCGATATTAGGTTGGATGAAACTGAAGA |
| | ATGA |
| 90 | MAPSVMASSATTVAPFQGSSPPPACRRPPSELQLRQRQHGGRIRCTGRATMGKPI |
| | PNPLLGLDSTGGVDLRTLGYSQQQQEKIKPKVRSTVAQHHEALVGHGFTHAHIV |
| | ALSQHPAALGTVAVKYQDMIAALPEATHEAIVGVGKRGAGARALEALLTVAGE |
| | LRGPPLQLDTGQLLKIAKRGGVTAVEAVHAWRNALTGAPLNLTPEQVVAIASH |
| | DGGKQALETVQRLLPVLCQAHGLTPEQVVAIASNGGGKQALETVQRLLPVLCQ |
| | AHGLTPEQVVAIASHDGGKQALETVQRLLPVLCQAHGLTPEQVVAIASHDGGK |
| | QALETVQRLLPVLCQAHGLTPEQVVAIASNIGGKQALETVQRLLPVLCQAHGLT |
| | PEQVVAIASHDGGKQALETVQRLLPVLCQAHGLTPEQVVAIASNIGGKQALETV |
| | QRLLPVLCQAHGLTPEQVVAIASNNGGKQALETVQRLLPVLCQAHGLTPEQVV |
| | AIASNIGGKQALETVQRLLPVLCQAHGLTPEQVVAIASNNGGKQALETVQRLLP |
| | VLCQAHGLTPEQVVAIASNIGGKQALETVQRLLPVLCQAHGLTPEQVVAIASNG |
| | GGKQALETVQRLLPVLCQAHGLTPEQVVAIASNIGGKQALETVQRLLPVLCQAH |
| | GLTPEQVVAIASNGGGKQALETVQRLLPVLCQAHGLTPEQVVAIASNNGGKQAL |
| | ETVQRLLPVLCQAHGLTPEQVVAIASNGGGKQALETVQRLLPVLCQAHGLTPEQ |
| | VVAIASNIGGKQALETVQRLLPVLCQAHGLTPQQVVAIASHDGGRPALESIVAQL |
| | SRPDPALAALTNDHLVALACLGGRPALDAVKKGLPHAPALIKRTGSGSMNELTL |
| | HPNVYATGASKGLIHVLERVWVREHTPGDGTIFVISGFGNYNGGVRFFESFREH |
| | KRRGGELVAVFAGSTSARLTSKQVVHEMLDCGADVHIVNRKRLLHAKCYGTSK |
| | MSGDSLIVTSGNFTGPGMSQNVEMSVLLDADTTAEMGFSWGTLLENLLDQRWN |
| | YHRPTLGDPANPAWSLLYDEQAADIRLDETEE |
| 91 | AGACATATATATCTATCTAAATTTATTGATATCAATATGAATGTGGAAAATG |
| | CTAAAATAACTTACATTGTGAAATGAAGGAAGTAATTTTCTTCACAAATTTTG |
| | GCACTTGCACTTACGGCTTGTGAAGCCCGCTCGATCTGAAGATTTTTCAAACG |
| | ATGCCATTCACCACGTGCCACTTTATATCAAACCTTATTTGTATCCAAGATTT |
| | CTGGTACTGGGCATTAGCGATGTCAAACAAACTCGACGTAAAGTTGAGGGAC |
| | AGAGTGTGAACTTATTTTATTCCCAGCAGAGAACGCCTCCAAGGTTTATGGG |
| | CTTTCTGGTCCATCGCAACGACACGGACTTTCTACAGGGCTGAGATTACGGG |
| | CCTAAATGGGCTCCCCCGTCTTGTGCTCCGCGCTGTTTCAAGTTGGGCCAAAA |
| | TGGAACTGGGCCGAATAGTCCGGTGCGGCTAACGGCATCCACAGCCAAGCC |
| | AACGGTGCCGTTCTCCCGCTCTCGCATTTCAAAAATCGAAAATAAAATCGGC |
| | AGGGCCGCCGACGACGCAACCACCCGTTGGGATCCAATCTACTCCTACGCAC |
| | CGAACAACTCATCGCACCCACCGATATGTGGGACCCAGCTGTAACGTGGGCC |
| | CACCGGCAAGCGGGACGTCTGCATCGGTCGCACCTACAACGAGGTGGCCAG |
| | GTCACCTACTGCTTTTTCTTGGAGCGACGCGTGCACTCGCTAACCGCACACGA |
| | AGGAAGCCTCTGGACCCACCTGTCAGTGAAACTAACGAGAGGCGGGTATGG |
| | ACTTCAACCGAGTGCAAACGAAATGGCCTCCTTGCGGTGGATATTATCAGAA |
| | AGCTCCCTAACTTATTAAATATTCTCGATATGATCCAACCACCCGACCTTTGC |
| | AGGCACGATGTGAGATGCGAGTGAATTTTGAAGACGTCAAAGGGGGTAAAA |
| | CGAAAAACTCCCCCATCCCTTCGCTTCCGCTCCTGAGAGGTCCAGGCTGTCTG |
| | GCCCTATAAAACGGGCGGAGTGCGGGGATCGCAAACCCTAAGCCGAGACAA |
| | CGCAGAGAAAGGCGTCTTCGTACTCGCCTCTCTCCGCGCCTCCGCGCTTTTCC |
| | TCCTCTCCCCTCTCTCCCTTCTCCGCCGCCGTCGCAGCATCAACCCAATCCGC |
| | CGCCAAGCTTCATCCAGGTACGGATCCGCGTCCCATCTCCCTCACCCCCCGTG |
| | TTCTTCGTGCCTGCTTCTGGGTCAGATCTGGGTGGATTCGCGGTTGTTGGATG |
| | TGGGGGGCTGTGTTTATTTGTCGGTGGATCTGGTTGTCTGGATCTGCGTTTTC |
| | TCTGTCGTAGTTAGCGGATCTGATGAAATGTTTAGTGTTCGTGTATACTGGTA |
| | TGGTGGATCTGGTCCTAGGATGCGTGGAATGGATATATGTAGGCGAATTGGA |
| | GGATTTATTTTGTGAATTTTGCTGAAATGATAGTTCTAAACACTGGATCTGAC |
| | CTCGGGATGCTGTTAAATGTGGAAATCATGGTCGATGCTGTCATGAACATGG |
| | TGTTCTTATGGTAGATCTGAGCAATGTATGTTTCAAAATTGTTTGTCACATGG |
| | AAATGCTATGGTTCTAGATGCAATAGAATGATACATGCCGAGATCCCCTCTA |
| | GTTGATATGATAGATCATGATGTTTTACAGCTATGTCATATGAATATGTTCAT |
| | TTGTTACCGATGTATTTGGATCTACTTAACATTTCCAAAGCACGCCGCGTTCT |
| | AATTCTAGATCTGGTAGTCATGTTTGTACACGTCATCCACCTAATACAAATAC |
| | ATATGTCTAGTGTTTGGTGACACTGCCCGTCAGATCTGTTTTTTCCAGATCTG |
| | TGGAACAAATACTCCATGCATGTATGGTAGTTTTGAAACGATCTTGTATCTTC |
| | CATTGTTGTAGTAACAACTAAATAAAGTACAATTGTTCAATTATTGGGAATC |
| | GTATTTTCTGTAGTGCCGATGTACAGCATATTCATAGATGTCTATTTAGGAAC |
| | TCAAATTTTAAATTGAGGACTAGTTATTTATTATGGGTCAGTCTTTTGAATTG |
| | TGTTATCTTGCTGTACTGAAATAATAATGTACCACTAAGGCGCTAACATGTAT |
| | TTTGTCCTTTCAGGCTGACTCGACGGATCCCGGGGGGCAAGAGCTCCTCCCC |
| | CCTCCCCCTCCGCCGCCGCCGGTAACCACCCCGCCCCTCTCCTCTTTCTTTCTC |
| | CGTTTTTTTTTTCGTCTCGGTCTCGATCTTTGGCCTTGGTAGTTTGGGTGGGCG |
| | AGAGCGGCTTCGTCGCCCAGATCGGTGCGCGGGAGGGGCGGGATCTCGCGG |
| | CTGGCGTCTCCGGGCGTGAGTCGGCCCGGATCCTCGCGGGGAATGGGGCTCT |
| | CGGATGTAGATCTTCTTTCTTTCTTCTTTTTGTGGTAGAATTTGAATCCCTCAG |
| | CATTGTTCATCGGTAGTTTTTCTTTTCATGATTTGTGACAAATGCAGCCTCGT |
| | GCGGAGCTTTTTTGTAGGTAGAAGATGGCCCCCTCCGTGATGGCGTCGTCGG |
| | CCACCACCGTCGCTCCCTTCCAGGGCTCAAGTCCACCGCCGGCTTGCCGTCGC |
| | CCGCCGTCCGAACTCCAGCTTCGGCAACGTCAGCATGGCGGCAGGATCAGAT |
| | GCACCGGCCGCGCCACCATGGGAAAACCTATTCCTAATCCTCTGCTGGGCCT |
| | GGATTCTACCGGAGGCGTGGACCTGAGAACACTGGGATATTCTCAGCAGCAG |
| | CAGGAGAAGATCAAGCCCAAGGTGAGATCTACAGTGGCCCAGCACCACGAA |
| | GCCCTGGTGGGACACGGATTTACACACGCCCACATTGTGGCCCTGTCTCAGC |
| | ACCCTGCCGCCCTGGGAACAGTGGCCGTGAAATATCAGGATATGATTGCCGC |
| | CCTGCCTGAGGCCACACACGAAGCCATTGTGGGAGTGGGAAAACGAGGCGC |
| | TGGAGCCAGAGCCCTGGAAGCCCTGCTGACAGTGGCCGGAGAACTGAGAGG |
| | ACCTCCTCTGCAGCTGGATACAGGACAGCTGCTGAAGATTGCCAAAAGGGGC |
| | GGAGTGACCGCGGTGGAAGCCGTGCACGCCTGGAGAAATGCCCTGACAGGA |
| | GCCCCTCTGAACCTGACCCCCGAACAGGTGGTGGCCATTGCCAGCCACGACG |
| | GCGGCAAGCAGGCCCTGGAAACCGTGCAGAGACTGCTGCCCGTGCTGTGCCA |
| | GGCCCATGGCCTGACACCTGAACAGGTGGTGGCTATCGCCTCTCACGACGGA |
| | GGAAAACAGGCTCTGGAAACAGTGCAGCGGCTGCTGCCTGTGCTGTGTCAGG |
| | CTCACGGCTTGACTCCAGAACAGGTGGTGGCTATTGCTTCCAACAACGGGGG |
| | GAAACAGGCCCTGGAAACTGTGCAGCGCCTGCTGCCAGTGCTGTGCCAGGCT |
| | CACGGACTGACCCCCGAACAGGTGGTGGCCATTGCCAGCAACATCGGCGGC |
| | AAGCAGGCCCTGGAAACCGTGCAGAGACTGCTGCCCGTGCTGTGCCAGGCCC |
| | ATGGCCTGACACCTGAACAGGTGGTGGCTATCGCCTCTAATATCGGAGGAAA |
| | ACAGGCTCTGGAAACAGTGCAGCGGCTGCTGCCTGTGCTGTGTCANGCTCAC |
| | GGCTTGACTCCAGAACAGGTGGTGGCTATTGCTTCCCACGACGGGGGGAAAC |
| | AGGCCCTGGGAACTGTGCAGCGCCTGCTGCCAGTGCTGTGCCAGGCTCACGG |
| | GCTGACCCCCGAACAGGTGGTGGCCATTGCCAGCAACATCGGCGGCAAGCA |
| | GGCCCTGGAAACCGTGCAGAGACTGCTGCCCGTGCTGTGCCAGGCCCATGGC |
| | CTGACACCTGAACAGGTGGTGGCTATCGCCTCTAACAACGGANGAAAACNG |
| | CTCTGGAAACAGTGCAGCGGCTGCTGCCTGTGCTGTGTCNNNTCACGGCTTTT |
| | GACTCCANAACAGGTGGTGGCTATTGCTTCCCACNACGGGGGGAAACAGGC |
| | CCTGGAAACTGTGCAGCNCCTGCTGCCAGTGCTGTGCCANGCTCACGGGCTG |
| | ACCCCCGAACAGGTGGTGGCCATTGCCAGCAACATCGGCGGCAAGCAGGCC |
| | CTGGAAACCGTGCAGAGACTGCTGCCCGTGCTGTGCCAGGCCCATGGCCTGA |
| | CACCTGAACAGGTGGTGGCTATCGCCTCTAACGGCGGAGGAAAACAGGCTCT |
| | GGAAACAGTGCAGCGGCTGCTGCCTGTGCTGTGTCAGGCTCACGGCTTGACT |
| | CCAGAACAGGTGGTGGCTATTGCTTCCAACGGCGGGGGGAAACAGGCCCTG |
| | GAAACTGTGCAGCGCCTGCTGCCAGTGCTGTGCCAGGCTCACGGACTGACCC |
| | CCGAACAGGTGGTGGCCATTGCCAGCAACATCGGCGGCAAGCAGGCCCTGG |
| | AAACCGTGCAGAGACTGCTGCCCGTGCTGTGCCAGGCCCATGGCCTGACACC |
| | TGAACAGGTGGTGGCTATCGCCTCTAACGGCGGAGGAAAACAAGCACTCGA |
| | GACAGTGCAGCGGCTGCTGCCTGTGCTGTGTCAGGCTCACGGCTTGACTCCA |
| | GAACAGGTGGTGGCTATTGCTTCCAACGGCGGGGGGAAACAGGCCCTGGAA |
| | ACTGTGCAGCGCCTGCTGCCAGTGCTGTGCCAGGCTCACGGCCTGACCCCCG |
| | AACAGGTGGTGGCCATTGCCAGCAACATCGGCGGCAAGCAGGCCCTGGAAA |
| | CCGTGCAGAGACTGCTGCCCGTGCTGTGCCAGGCCCATGGCCTGACACCTGA |
| | ACAGGTGGTGGCTATCGCCTCTAACGGCGGAGGAAAACAGGCTCTGGAAAC |
| | AGTGCAGCGGCTGCTGCCTGTGCTGTGTCAGGCTCACGGCTTGACTCCACAA |
| | CAGGTCGTGGCAATTGCTANCCACGACGGCNGANNGCCCGCCCTGGANANC |
| | ATTNTGGNCCTGCTGTCNATACCGGACCCAGCTCTCGCTTCTAGACCGGACC |
| | CAGCTCTCGCTGCATTGACTAACGACCATCTCGTCGCTTTGGCTTGCCTCGGT |
| | GGAAGGCCAGCTCTTGACGCTGTAAAGAAAGGTTTGCCACATGCTCCAGCTC |
| | TTATTAAAAGGACTGGTAGCGGTAGCATGAATGAATTAACCTTGCACCCTAA |
| | CGTCTACGCAACAGGAGCCTCCAAGGGTTTGATACACGTATTGGAAAGGGTG |
| | TGGGTAAGGGAGCATACTCCAGGTGATGGAACAATATTTGTCATTAGCGGAT |
| | TCGGAAATTATAATGGAGGTGTAAGGTTCTTTGAATCCTTCAGGGAGCATAA |
| | ACGCAGGGGAGGTGAATTGGTTGCTGTCTTTGCAGGATCAACCTCGGCTAGG |
| | TTGACTTCTAAACAAGTAGTCCATGAAATGTTGGACTGTGGTGCAGACGTAC |
| | ATATTGTAAATCGCAAAAGGTTGCTTCATGCAAAATGCTATGGTACGTCCAA |
| | AATGAGCGGAGACAGCTTGATAGTCACAAGCGGTAACTTTACAGGGCCTGGT |
| | ATGAGCCAAAATGTCGAGATGAGCGTGCTCTTGGATGCTGACACGACTGCGG |
| | AAATGGGATTTAGCTGGGGTACACTCTTGGAAAATCTGTTGGACCAAAGGTG |
| | GAATTATCACAGACCGACGCTCGGCGACCCTGCGAACCCTGCATGGTCGTTG |
| | CTGTACGACGAACAAGCTGCGGACATACGCCTCGACGAGACAGAGGAATGA |
| | GAATTCATATGAAGATGAAGATGAAATATTTGGTGTGTCAAATAAAAAGCTT |
| | GTGTGCTTAAGTTTGTGTTTTTTTCTTGGCTTGTTGTGTTATGAATTTGTGGCT |
| | TTTTCTAATATTAAATGAATGTAAGATCTCATTATAATGAATAAACAAATGTT |
| | TCTATAATCCATTGTGAATGTTTTGTTGGATCTCTTCTGCAGCATATAACTAC |
| | TGTATGTGCTATGGTATGGACTATGGAATATGATTAAAGATAAG |
| 92 | CTGCAGTGCAGCGTGACCCGGTCGTGCCCCTCTCTAGAGATAATGAGCATTG |
| | CATGTCTAAGTTATAAAAAATTACCACATATTTTTTTTGTCACACTTGTTTGA |
| | AGTGCAGTTTATCTATCTTTATACATATATTTAAACTTTACTCTACGAATAAT |
| | ATAATCTATAGTACTACAATAATATCAGTGTTTTAGAGAATCATATAAATGA |
| | ACAGTTAGACATGGTCTAAAGGACAATTGAGTATTTTGACAACAGGACTCTA |
| | CAGTTTTATCTTTTTAGTGTGCATGTGTTCTCCTTTTTTTTTGCAAATAGCTTC |
| | ACCTATATAATACTTCATCCATTTTATTAGTACATCCATTTAGGGTTTAGGGT |
| | TAATGGTTTTTATAGACTAATTTTTTTAGTACATCTATTTTATTCTATTTTAGC |
| | CTCTAAATTAAGAAAACTAAAACTCTATTTTAGTTTTTTTATTTAATAATTTA |
| | GATATAAAATAGAATAAAATAAAGTGACTAAAAATTAAACAAATACCCTTTA |
| | AGAAATTAAAAAAACTAAGGAAACATTTTTCTTGTTTCGAGTAGATAATGCC |
| | AGCCTGTTAAACGCCGTCGACGAGTCTAACGGACACCAACCAGCGAACCAG |
| | CAGCGTCGCGTCGGGCCAAGCGAAGCAGACGGCACGGCATCTCTGTCGCTGC |
| | CTCTGGACCCCTCTCGAGAGTTCCGCTCCACCGTTGGACTTGCTCCGCTGTCG |
| | GCATCCAGAAATTGCGTGGCGGAGCGGCAGACGTGAGCCGGCACGGCAGGC |
| | GGCCTCCTCCTCCTCTCACGGCACCGGCAGCTACGGGGGATTCCTTTCCCACC |
| | GCTCCTTCGCTTTCCCTTCCTCGCCCGCCGTAATAAATAGACACCCCCTCCAC |
| | ACCCTCTTTCCCCAACCTCGTGTTGTTCGGAGCGCACACACACACAACCAGA |
| | TCTCCCCCAAATCCACCCGTCGGCACCTCCGCTTCAAGGTACGCCGCTCGTCC |
| | TCCCCCCCCCCCCCTCTCTACCTTCTCTAGATCGGCGTTCCGGTCCATGGTTA |
| | GGGCCCGGTAGTTCTACTTCTGTTCATGTTTGTGTTAGATCCGTGTTTGTGTTA |
| | GATCCGTGCTGCTAGCGTTCGTACACGGATGCGACCTGTACGTCAGACACGT |
| | TCTGATTGCTAACTTGCCAGTGTTTCTCTTTGGGGAATCCTGGGATGGCTCTA |
| | GCCGTTCCGCAGACGGGATCGATTTCATGATTTTTTTTGTTTCGTTGCATAGG |
| | GTTTGGTTTGCCCTTTTCCTTTATTTCAATATATGCCGTGCACTTGTTTGTCGG |
| | GTCATCTTTTCATGCTTTTTTTTGTCTTGGTTGTGATGATGTGGTCTGGTTGGG |
| | CGGTCGTTCTAGATCGGAGTAGAATTAATTCTGTTTCAAACTACCTGGTGGAT |
| | TTATTAATTTTGGATCTGTATGTGTGTGCCATACATATTCATAGTTACGAATT |
| | GAAGATGATGGATGGAAATATCGATCTAGGATAGGTATACATGTTGATGCGG |
| | GTTTTACTGATGCATATACAGAGATGCTTTTTGTTCGCTTGGTTGTGATGATG |
| | TGGTGTGGTTGGGCGGTCGTTCATTCGTTCTAGATCGGAGTAGAATACTGTTT |
| | CAAACTACCTGGTGTATTTATTAATTTTGGAACTGTATGTGTGTGTCATACAT |
| | CTTCATAGTTACGAGTTTAAGATGGATGGAAATATCGATCTAGGATAGGTAT |
| | ACATGTTGATGTGGGTTTTACTGATGCATATACATGATGGCATATGCAGCATC |
| | TATTCATATGCTCTAACCTTGAGTACCTATCTATTATAATAAACAAGTATGTT |
| | TTATAATTATTTTGATCTTGATATACTTGGATGATGGCATATGCAGCAGCTAT |
| | ATGTGGATTTTTTTAGCCCTGCCTTCATACGCTATTTATTTGCTTGGTACTGTT |
| | TCTTTTGTCGATGCTCACCCTGTTGTTTGGTGTTACTTCTGCAGGTCGACTCTA |
| | GAGGATCCATGGCTCCCAGCGTCATGGCCTCCAGCGCGACAACTGTGGCCCC |
| | GTTTCAGGGTTCGAGCCCGCCACCTGCTTGCCGCCGGCCACCTAGCGAGCTG |
| | CAACTCCGCCAGCGCCAACACGGAGGGAGAATTAGATGCACCGGCCGCGCC |
| | ACCATGGGAAAACCGATCCCAAACCCGCTTTTGGGGCTCGACAGCACAGGA |
| | GGGGTTGATCTCAGGACCCTCGGTTACAGCCAACAGCAACAAGAAAAAATT |
| | AAGCCTAAAGTGAGGTCCACGGTAGCTCAACATCACGAGGCTTTGGTAGGGC |
| | ATGGCTTCACGCACGCTCATATCGTCGCTCTCAGCCAACATCCCGCTGCGCTT |
| | GGGACGGTCGCTGTTAAGTACCAAGACATGATCGCTGCGCTTCCGGAAGCTA |
| | CGCATGAGGCGATCGTCGGTGTAGGGAAGCGGGGAGCAGGTGCGAGGGCGC |
| | TCGAGGCGCTCTTGACCGTCGCGGGTGAGCTTAGGGGTCCGCCACTCCAATT |
| | GGACACCGGACAACTTTTGAAAATCGCTAAGAGAGGAGGCGTCACAGCCGT |
| | CGAGGCGGTGCACGCCTGGAGAAATGCCCTGACAGGAGCCCCTCTGAACCTG |
| | ACCCCCGAACAGGTGGTGGCCATTGCCAGCCACGACGGCGGCAAGCAGGCC |
| | CTGGAAACCGTGCAGAGACTGCTGCCCGTGCTGTGCCAGGCCCATGGCCTGA |
| | CACCTGAACAGGTGGTGGCTATCGCCTCTAACGGCGGAGGAAAACAGGCTCT |
| | GGAAACAGTGCAGCGGCTGCTGCCTGTGCTGTGTCAGGCTCACGGCTTGACT |
| | CCAGAACAGGTGGTGGCTATTGCTTCCCACGACGGGGGGAAACAGGCCCTG |
| | GAAACTGTGCAGCGCCTGCTGCCAGTGCTGTGCCAGGCTCACGGACTGACCC |
| | CCGAACAGGTGGTGGCCATTGCCAGCCACGACGGCGGCAAGCAGGCCCTGG |
| | AAACCGTGCAGAGACTGCTGCCCGTGCTGTGCCAGGCCCATGGCCTGACACC |
| | TGAACAGGTGGTGGCTATCGCCTCTAATATCGGAGGAAAACAGGCTCTGGAA |
| | ACAGTGCAGCGGCTGCTGCCTGTGCTGTGTCAGGCTCACGGCTTGACTCCAG |
| | AACAGGTGGTGGCTATTGCTTCCCACGACGGGGGGAAACAGGCCCTGGAAA |
| | CTGTGCAGCGCCTGCTGCCAGTGCTGTGCCAGGCTCACGGGCTGACCCCCGA |
| | ACAGGTGGTGGCCATTGCCAGCAACATCGGCGGCAAGCAGGCCCTGGAAAC |
| | CGTGCAGAGACTGCTGCCCGTGCTGTGCCAGGCCCATGGCCTGACACCTGAA |
| | CAGGTGGTGGCTATCGCCTCTAACAACGGAGGAAAACAGGCTCTGGAAACA |
| | GTGCAGCGGCTGCTGCCTGTGCTGTGTCAGGCTCACGGCTTGACTCCAGAAC |
| | AGGTGGTGGCTATTGCTTCCAATATTGGGGGGAAACAGGCCCTGGAAACTGT |
| | GCAGCGCCTGCTGCCAGTGCTGTGCCAGGCTCACGGCCTCACTCCCGAACAG |
| | GTGGTGGCCATTGCCAGCAACAACGGCGGCAAGCAGGCCCTGGAAACCGTG |
| | CAGAGACTGCTGCCCGTGCTGTGCCAGGCCCATGGCCTGACACCTGAACAGG |
| | TGGTGGCTATCGCCTCTAATATCGGAGGAAAACAGGCTCTGGAAACAGTGCA |
| | GCGGCTGCTGCCTGTGCTGTGTCAGGCTCACGGCTTGACTCCAGAACAGGTG |
| | GTGGCTATTGCTTCCAACGGCGGGGGGAAACAGGCCCTGGAAACTGTGCAGC |
| | GCCTGCTGCCAGTGCTGTGCCAGGCTCACGGACTGACCCCCGAACAGGTGGT |
| | GGCCATTGCCAGCAACATCGGCGGCAAGCAGGCCCTGGAAACCGTGCAGAG |
| | ACTGCTGCCCGTGCTGTGCCAGGCCCATGGCCTGACACCTGAACAGGTGGTG |
| | GCTATCGCCTCTAACGGCGGAGGAAAACAAGCACTCGAGACAGTGCAGCGG |
| | CTGCTGCCTGTGCTGTGTCAGGCTCACGGCTTGACTCCAGAACAGGTGGTGG |
| | CTATTGCTTCCAACAACGGGGGGAAACAGGCCCTGGAAACTGTGCAGCGCCT |
| | GCTGCCAGTGCTGTGCCAGGCTCACGGCCTGACCCCCGAACAGGTGGTGGCC |
| | ATTGCCAGCAACGGCGGCGGCAAGCAGGCCCTGGAAACCGTGCAGAGACTG |
| | CTGCCCGTGCTGTGCCAGGCCCATGGCCTGACACCTGAACAGGTGGTGGCTA |
| | TCGCCTCTAATATCGGAGGAAAACAGGCTCTGGAAACAGTGCAGCGGCTGCT |
| | GCCTGTGCTGTGTCAGGCTCACGGCTTGACTCCACAGCAGGTCGTGGCAATT |
| | GCTAGCCACGACGGCGGACGGCCCGCCCTGGAGAGCATTGTGGCCCAGCTGT |
| | CTAGACCTGATCCTGCCCTGGCCGCCCTGACAAATGATCACCTGGTGGCCCT |
| | GGCCTGTCTGGGAGGCAGACCTGCCCTGGATGCCGTGAAAAAAGGACTGCCT |
| | CACGCCCCTGCCCTGATCAAGAGAACAGGATCCGGATCCATGAACGAGTTAA |
| | CGCTGCATCCGAATGTGTATGCGACCGGCGCGAGTAAAGGGCTGATACATGT |
| | GCTGGAGCGCGTATGGGTTCGAGAACACACGCCTGGGGACGGCACGATCTTC |
| | GTGATATCTGGCTTTGGCAACTACAACGGTGGCGTTCGCTTCTTCGAGTCGTT |
| | TCGTGAACACAAGAGACGTGGCGGGGAGCTGGTAGCGGTGTTCGCTGGCAG |
| | CACAAGCGCGCGACTGACAAGCAAACAGGTTGTTCACGAGATGCTTGATTGC |
| | GGGGCGGATGTTCACATAGTGAACAGAAAGCGACTCCTTCACGCTAAGTGTT |
| | ACGGCACAAGCAAGATGTCAGGTGATTCCCTAATTGTAACCTCAGGCAATTT |
| | CACAGGTCCGGGAATGTCGCAGAACGTGGAAATGTCTGTATTGCTAGACGCA |
| | GATACAACCGCCGAGATGGGGTTCTCGTGGGGCACGTTGCTTGAGAACTTGC |
| | TTGATCAGCGATGGAACTACCATCGCCCCACCTTAGGCGATCCAGCAAATCC |
| | AGCCTGGAGCCTGCTTTATGATGAGCAGGCAGCCGATATTAGGTTGGATGAA |
| | ACTGAAGAATGAGAATTCGGTACCGAGTTTGAATCAAATCTTCACTTGTTTA |
| | ATGAGATATGCGAGACGCCTATGATCGCATGATATTTGCTTTCAATTCTGTTG |
| | TGCACGTTGTAAAAAACCTGAGCATGTGTAGCTCAGATCCTTACCGCCGGTT |
| | TCGGTTCATTCTAATGAATATATCACCCGTTACTATCGTATTTTTATGAATAA |
| | TATTCTCCGTTCAATTTACTGATTGTACCCTACTACTTATATGTACAATATTAA |
| | AATGAAAACAATATATTGTGCTGAATAGGTTTATAGCGACATCTATGATAGA |
| | GCGCCACAATAACAAACAATTGCGTTTTATTATTACAAATCCAATTTTAAAA |
| | AAAGCGGCAGAACCGGTCAAACCTAAAAGACTGATTACATAAATCTTATTCA |
| | AATTTCAAAAGTGCCCCAGGGGCTAGTATCTACGACACACCGAGCGGCGAAT |
| | TCAGTACATTAAAAACGTCCGCAATGTGTTATTAAGTTGTCTAAGCGTCAATT |
| | TGTTTACACCACAATATATCCTGCCACCAGCCAGCCAACAGCTCCCCGACCG |
| | GCAGCTCGGCACAAAATCACCACTCGATACAGGCAGCCCATCAGTCCGGGAC |
| | GGCGTCAGCGGGA |
| 93 | CCAGTTTCGATTATTCTCCTCAAAGGATTCTATCTTAAAACACACATTGCTAA |
| | CACTAGAAAACTTCCCTTTTCTTTCTATTGAAATGAAAAAGGAGAAAAGTGG |
| | ATTTCTAGTCACAAGCTACAAAATTAAGAACAAATTGGAACCATTAACTAGA |
| | ATTCTCTTTTTATTTTGAATTTCGGTATTCTCTCCCGCCTGCCATTTAATGTCA |
| | TAATAAAAGACAATGGATTTATGCCTAATCCGTATATAGGTAAACTCCAGGT |
| | CCGAACAGCATTATTATCTATGGATCCCCCTTATGTACATATCTCTGTGGAGA |
| | ATCGTTCTTTAATTTTTCATTGCATTAAATATCTTGAATAAAAAAAAGAAAA |
| 94 | ACACACATTGCTAACACTAGAAAACTTCC |
| 95 | CGGACCTGGAGTTTACCTATATACGGA |
| 96 | MDPIKTGRKNHQRHHVLPARGRVGAAAVRCSAVSPVTPPSPAPPATPLRPWGPA |
| | EPRKGADILVEALERCGVSDVFAYPGGASMEIHQALTRSPVITNHLFRHEQGEAF |
| | AASGYARASGRVGVCVATSGPGATNLVSALADALLDSVPMVAITGQVPRRMIG |
| | TDAFQETPIVEVTRSITKHNYLVLDVEDIPRVIQEAFFLASSGRPGPVLVDIPKDIQ |
| | QQMAVPVWDTSMNLPGYIARLPKPPATELLEQVLRLVGESRRPILYVGGGCSAS |
| | GDELRWFVELTGIPVTTTLMGLGNFPSDDPLSLRMLGMHGTVYANYAVDKADL |
| | LLAFGVRFDDRVTGKIEAFASRAKIVHIDIDPAEIGKNKQPHVSICADVKLALQGL |
| | NALLQQSTTKTSSDFSAWHNELDQQKREFPLGYKTFGEEIPPQYAIQVLDELTKG |
| | EAIIATGVGQHQMWAAQYYTYKRPRQWLSSAGLGAMGFGLPAAAGASVANPG |
| | VTVVDIDGDGSFLMNIQELALIRIENLPVKVMVLNNQHLGMVVQLEDRFYKAN |
| | RAHTYLGNPECESEIYPDFVTIAKGFNIPAVRVTKKSEVRAAIKKMLETPGPYLL |
| | DIIVPHQEHVLPMIPIGGAFKDMILDGDGRTVY |
| 97 | MIPEKTGRKNHQRHHVLPARGRVGAAAVRCSAVSPVTPPSPAPPATPLRPWGPA |
| | EPRKGADILVEALERCGVSDVFAYPGGASMEIHQALTRSPVITNHLFRHEQGEAF |
| | AASGYARASGRVGVCVATSGPGATNLVSALADALLDSVPMVAITGQVPRRMIG |
| | TDAFQETPIVEVTRSITKHNYLVLDVEDIPRVIQEAFFLASSGRPGPVLVDIPKDIQ |
| | QQMAVPVWDTSMNLPGYIARLPKPPATELLEQVLRLVGESRRPILYVGGGCSAS |
| | GDELRWFVELTGIPVTTTLMGLGNFPSDDPLSLRMLGMHGTVYANYAVDKADL |
| | LLAFGVRFDDRVTGKIEAFASRAKIVHIDIDPAEIGKNKQPHVSICADVKLALQGL |
| | NALLQQSTTKTSSDFSAWHNELDQQKREFPLGYKTFGEEIPPQYAIQVLDELTKG |
| | EAIIATGVGQHQMWAAQYYTYKRPRQWLSSAGLGAMGFGLPAAAGASVANPG |
| | VTVVDIDGDGSFLMNIQELALIRIENLPVKVMVLNNQHLGMVVQLEDRFYKAN |
| | RAHTYLGNPECESEIYPDFVTIAKGFNIPAVRVTKKSEVRAAIKKMLETPGPYLL |
| | DIIVPHQEHVLPMIPIGGAFKDMILDGDGRTVY |
| 98 | MKTGRKNHQRHHVLPARGRVGAAAVRCSAVSPVTPPSPAPPATPLRPWGPAEP |
| | RKGADILVEALERCGVSDVFAYPGGASMEIHQALTRSPVITNHLFRHEQGEAFA |
| | ASGYARASGRVGVCVATSGPGATNLVSALADALLDSVPMVAITGQVPRRMIGT |
| | DAFQETPIVEVTRSITKHNYLVLDVEDIPRVIQEAFFLASSGRPGPVLVDIPKDIQQ |
| | QMAVPVWDTSMNLPGYIARLPKPPATELLEQVLRLVGESRRPILYVGGGCSASG |
| | DELRWFVELTGIPVTTTLMGLGNFPSDDPLSLRMLGMHGTVYANYAVDKADLL |
| | LAFGVRFDDRVTGKIEAFASRAKIVHIDIDPAEIGKNKQPHVSICADVKLALQGL |
| | NALLQQSTTKTSSDFSAWHNELDQQKREFPLGYKTFGEEIPPQYAIQVLDELTKG |
| | EAIIATGVGQHQMWAAQYYTYKRPRQWLSSAGLGAMGFGLPAAAGASVANPG |
| | VTVVDIDGDGSFLMNIQELALIRIENLPVKVMVLNNQHLGMVVQLEDRFYKAN |
| | RAHTYLGNPECESEIYPDFVTIAKGFNIPAVRVTKKSEVRAAIKKMLETPGPYLL |
| | DIIVPHQEHVLPMIPIGGAFKDMILDGDGRTVY |
| 99 | TCGGTAATCCGAAGCCCA |
| 100 | GCAAGATCCTCAAGAAAA |
| 101 | ATGGCAGCGAAGATCCGGATCGTCATGAAGTCCTTCATGAGCCAGGCCAATA |
| | AGGTGGAGGGCGTCATCCCGTATGCCCAAAAAGTCGGGCTCCCAGAGTCGCG |
| | CTCTCTCTACACAGTCTTGCGGTCGCCACATATCGATAAAAAGTCCAGAGAA |
| | CAATTCTCTATGACCGGCCGCGCCACCATGGGAAAACCTATTCCTAATCCTCT |
| | GCTGGGCCTGGATTCTACCGGAGGCGTGGACCTGAGAACACTGGGATATTCT |
| | CAGCAGCAGCAGGAGAAGATCAAGCCCAAGGTGAGATCTACAGTGGCCCAG |
| | CACCACGAAGCCCTGGTGGGACACGGATTTACACACGCCCACATTGTGGCCC |
| | TGTCTCAGCACCCTGCCGCCCTGGGAACAGTGGCCGTGAAATATCAGGATAT |
| | GATTGCCGCCCTGCCTGAGGCCACACACGAAGCCATTGTGGGAGTGGGAAA |
| | ACGAGGCGCTGGAGCCAGAGCCCTGGAAGCCCTGCTGACAGTGGCCGGAGA |
| | ACTGAGAGGACCTCCTCTGCAGCTGGATACAGGACAGCTGCTGAAGATTGCC |
| | AAAAGGGGCGGAGTGACCGCGGTGGAAGCCGTGCACGCCTGGAGAAATGCC |
| | CTGACAGGAGCCCCTCTGAACCTGACCCCCGAACAGGTGGTGGCCATTGCCA |
| | GCAACGGCGGCGGCAAGCAGGCCCTGGAAACCGTGCAGAGACTGCTGCCCG |
| | TGCTGTGCCAGGCCCATGGCCTGACACCTGAACAGGTGGTGGCTATCGCCTC |
| | TCACGACGGAGGAAAACAGGCTCTGGAAACAGTGCAGCGGCTGCTGCCTGT |
| | GCTGTGTCAGGCTCACGGCTTGACTCCAGAACAGGTGGTGGCTATTGCTTCC |
| | AACAACGGGGGGAAACAGGCCCTGGAAACTGTGCAGCGCCTGCTGCCAGTG |
| | CTGTGCCAGGCTCACGGACTGACCCCCGAACAGGTGGTGGCCATTGCCAGCA |
| | ACAACGGCGGCAAGCAGGCCCTGGAAACCGTGCAGAGACTGCTGCCCGTGC |
| | TGTGCCAGGCCCATGGCCTGACACCTGAACAGGTGGTGGCTATCGCCTCTAA |
| | CGGCGGAGGAAAACAGGCTCTGGAAACAGTGCAGCGGCTGCTGCCTGTGCT |
| | GTGTCAGGCTCACGGCTTGACTCCAGAACAGGTGGTGGCTATTGCTTCCAAT |
| | ATTGGGGGGAAACAGGCCCTGGAAACTGTGCAGCGCCTGCTGCCAGTGCTGT |
| | GCCAGGCTCACGGGCTGACCCCCGAACAGGTGGTGGCCATTGCCAGCAACAT |
| | CGGCGGCAAGCAGGCCCTGGAAACCGTGCAGAGACTGCTGCCCGTGCTGTGC |
| | CAGGCCCATGGCCTGACACCTGAACAGGTGGTGGCTATCGCCTCTAACGGCG |
| | GAGGAAAACAGGCTCTGGAAACAGTGCAGCGGCTGCTGCCTGTGCTGTGTCA |
| | GGCTCACGGCTTGACTCCAGAACAGGTGGTGGCTATTGCTTCCCACGACGGG |
| | GGGAAACAGGCCCTGGAAACTGTGCAGCGCCTGCTGCCAGTGCTGTGCCAGG |
| | CTCACGGCCTCACTCCCGAACAGGTGGTGGCCATTGCCAGCCACGACGGCGG |
| | CAAGCAGGCCCTGGAAACCGTGCAGAGACTGCTGCCCGTGCTGTGCCAGGCC |
| | CATGGCCTGACACCTGAACAGGTGGTGGCTATCGCCTCTAACAACGGAGGAA |
| | AACAGGCTCTGGAAACAGTGCAGCGGCTGCTGCCTGTGCTGTGTCAGGCTCA |
| | CGGCTTGACTCCAGAACAGGTGGTGGCTATTGCTTCCAATATTGGGGGGAAA |
| | CAGGCCCTGGAAACTGTGCAGCGCCTGCTGCCAGTGCTGTGCCAGGCTCACG |
| | GACTGACCCCCGAACAGGTGGTGGCCATTGCCAGCAACATCGGCGGCAAGC |
| | AGGCCCTGGAAACCGTGCAGAGACTGCTGCCCGTGCTGTGCCAGGCCCATGG |
| | CCTGACACCTGAACAGGTGGTGGCTATCGCCTCTAACAACGGAGGAAAACA |
| | AGCACTCGAGACAGTGCAGCGGCTGCTGCCTGTGCTGTGTCAGGCTCACGGC |
| | TTGACTCCAGAACAGGTGGTGGCTATTGCTTCCCACGACGGGGGGAAACAGG |
| | CCCTGGAAACTGTGCAGCGCCTGCTGCCAGTGCTGTGCCAGGCTCACGGCCT |
| | GACCCCCGAACAGGTGGTGGCCATTGCCAGCCACGACGGCGGCAAGCAGGC |
| | CCTGGAAACCGTGCAGAGACTGCTGCCCGTGCTGTGCCAGGCCCATGGCCTG |
| | ACACCTGAACAGGTGGTGGCTATCGCCTCTCACGACGGAGGAAAACAGGCTC |
| | TGGAAACAGTGCAGCGGCTGCTGCCTGTGCTGTGTCAGGCTCACGGCTTGAC |
| | TCCACAGCAGGTCGTGGCAATTGCTAGCAATATCGGCGGACGGCCCGCCCTG |
| | GAGAGCATTGTGGCCCAGCTGTCTAGACCGGACCCAGCTCTCGCTGCATTGA |
| | CTAACGACCATCTCGTCGCTTTGGCTTGCCTCGGTGGAAGGCCAGCTCTTGAC |
| | GCTGTAAAGAAAGGTTTGCCACATGCTCCAGCTCTTATTAAAAGGACTGGTA |
| | GCGGTAGCATGAATGAATTAACCTTGCACCCTAACGTCTACGCAACAGGAGC |
| | CTCCAAGGGTTTGATACACGTATTGGAAAGGGTGTGGGTAAGGGAGCATACT |
| | CCAGGTGATGGAACAATATTTGTCATTAGCGGATTCGGAAATTATAATGGAG |
| | GTGTAAGGTTCTTTGAATCCTTCAGGGAGCATAAACGCAGGGGAGGTGAATT |
| | GGTTGCTGTCTTTGCAGGATCAACCTCGGCTAGGTTGACTTCTAAACAAGTA |
| | GTCCATGAAATGTTGGACTGTGGTGCAGACGTACATATTGTAAATCGCAAAA |
| | GGTTGCTTCATGCAAAATGCTATGGTACGTCCAAAATGAGCGGAGACAGCTT |
| | GATAGTCACAAGCGGTAACTTTACAGGGCCTGGTATGAGCCAAAATGTCGAG |
| | ATGAGCGTGCTCTTGGATGCTGACACGACTGCGGAAATGGGATTTAGCTGGG |
| | GTACACTCTTGGAAAATCTGTTGGACCAAAGGTGGAATTATCACAGACCGAC |
| | GCTCGGCGACCCTGCGAACCCTGCATGGTCGTTGCTGTACGACGAACAAGCT |
| | GCGGACATACGCCTCGACGAGACAGAGGAATGA |
| 102 | MAAKIRIVMKSFMSQANKVEGVIPYAQKVGLPESRSLYTVLRSPHIDKKSREQFS |
| | MTGRATMGKPIPNPLLGLDSTGGVDLRTLGYSQQQQEKIKPKVRSTVAQHHEAL |
| | VGHGFTHAHIVALSQHPAALGTVAVKYQDMIAALPEATHEAIVGVGKRGAGAR |
| | ALEALLTVAGELRGPPLQLDTGQLLKIAKRGGVTAVEAVHAWRNALTGAPLNL |
| | TPEQVVAIASNGGGKQALETVQRLLPVLCQAHGLTPEQVVAIASHDGGKQALET |
| | VQRLLPVLCQAHGLTPEQVVAIASNNGGKQALETVQRLLPVLCQAHGLTPEQV |
| | VAIASNNGGKQALETVQRLLPVLCQAHGLTPEQVVAIASNGGGKQALETVQRLL |
| | PVLCQAHGLTPEQVVAIASNIGGKQALETVQRLLPVLCQAHGLTPEQVVAIASNI |
| | GGKQALETVQRLLPVLCQAHGLTPEQVVAIASNGGGKQALETVQRLLPVLCQA |
| | HGLTPEQVVAIASHDGGKQALETVQRLLPVLCQAHGLTPEQVVAIASHDGGKQ |
| | ALETVQRLLPVLCQAHGLTPEQVVAIASNNGGKQALETVQRLLPVLCQAHGLTP |
| | EQVVAIASNIGGKQALETVQRLLPVLCQAHGLTPEQVVAIASNIGGKQALETVQ |
| | RLLPVLCQAHGLTPEQVVAIASNNGGKQALETVQRLLPVLCQAHGLTPEQVVAI |
| | ASHDGGKQALETVQRLLPVLCQAHGLTPEQVVAIASHDGGKQALETVQRLLPV |
| | LCQAHGLTPEQVVAIASHDGGKQALETVQRLLPVLCQAHGLTPQQVVAIASNIG |
| | GRPALESIVAQLSRPDPALAALTNDHLVALACLGGRPALDAVKKGLPHAPALIK |
| | RTGSGSMNELTLHPNVYATGASKGLIHVLERVWVREHTPGDGTIFVISGFGNYN |
| | GGVRFFESFREHKRRGGELVAVFAGSTSARLTSKQVVHEMLDCGADVHIVNRK |
| | RLLHAKCYGTSKMSGDSLIVTSGNFTGPGMSQNVEMSVLLDADTTAEMGFSWG |
| | TLLENLLDQRWNYHRPTLGDPANPAWSLLYDEQAADIRLDETEE |
| 103 | AGACATATATATCTATCTAAATTTATTGATATCAATATGAATGTGGAAAATG |
| | CTAAAATAACTTACATTGTGAAATGAAGGAAGTAATTTTCTTCACAAATTTTG |
| | GCACTTGCACTTACGGCTTGTGAAGCCCGCTCGATCTGAAGATTTTTCAAACG |
| | ATGCCATTCACCACGTGCCACTTTATATCAAACCTTATTTGTATCCAAGATTT |
| | CTGGTACTGGGCATTAGCGATGTCAAACAAACTCGACGTAAAGTTGAGGGAC |
| | AGAGTGTGAACTTATTTTATTCCCAGCAGAGAACGCCTCCAAGGTTTATGGG |
| | CTTTCTGGTCCATCGCAACGACACGGACTTTCTACAGGGCTGAGATTACGGG |
| | CCTAAATGGGCTCCCCCGTCTTGTGCTCCGCGCTGTTTCAAGTTGGGCCAAAA |
| | TGGAACTGGGCCGAATAGTCCGGTGCGGCTAACGGCATCCACAGCCAAGCC |
| | AACGGTGCCGTTCTCCCGCTCTCGCATTTCAAAAATCGAAAATAAAATCGGC |
| | AGGGCCGCCGACGACGCAACCACCCGTTGGGATCCAATCTACTCCTACGCAC |
| | CGAACAACTCATCGCACCCACCGATATGTGGGACCCAGCTGTAACGTGGGCC |
| | CACCGGCAAGCGGGACGTCTGCATCGGTCGCACCTACAACGAGGTGGCCAG |
| | GTCACCTACTGCTTTTTCTTGGAGCGACGCGTGCACTCGCTAACCGCACACGA |
| | AGGAAGCCTCTGGACCCACCTGTCAGTGAAACTAACGAGAGGCGGGTATGG |
| | ACTTCAACCGAGTGCAAACGAAATGGCCTCCTTGCGGTGGATATTATCAGAA |
| | AGCTCCCTAACTTATTAAATATTCTCGATATGATCCAACCACCCGACCTTTGC |
| | AGGCACGATGTGAGATGCGAGTGAATTTTGAAGACGTCAAAGGGGGTAAAA |
| | CGAAAAACTCCCCCATCCCTTCGCTTCCGCTCCTGAGAGGTCCAGGCTGTCTG |
| | GCCCTATAAAACGGGCGGAGTGCGGGGATCGCAAACCCTAAGCCGAGACAA |
| | CGCAGAGAAAGGCGTCTTCGTACTCGCCTCTCTCCGCGCCTCCGCGCTTTTCC |
| | TCCTCTCCCCTCTCTCCCTTCTCCGCCGCCGTCGCAGCATCAACCCAATCCGC |
| | CGCCAAGCTTCATCCAGGTACGGATCCGCGTCCCATCTCCCTCACCCCCCGTG |
| | TTCTTCGTGCCTGCTTCTGGGTCAGATCTGGGTGGATTCGCGGTTGTTGGATG |
| | TGGGGGGCTGTGTTTATTTGTCGGTGGATCTGGTTGTCTGGATCTGCGTTTTC |
| | TCTGTCGTAGTTAGCGGATCTGATGAAATGTTTAGTGTTCGTGTATACTGGTA |
| | TGGTGGATCTGGTCCTAGGATGCGTGGAATGGATATATGTAGGCGAATTGGA |
| | GGATTTATTTTGTGAATTTTGCTGAAATGATAGTTCTAAACACTGGATCTGAC |
| | CTCGGGATGCTGTTAAATGTGGAAATCATGGTCGATGCTGTCATGAACATGG |
| | TGTTCTTATGGTAGATCTGAGCAATGTATGTTTCAAAATTGTTTGTCACATGG |
| | AAATGCTATGGTTCTAGATGCAATAGAATGATACATGCCGAGATCCCCTCTA |
| | GTTGATATGATAGATCATGATGTTTTACAGCTATGTCATATGAATATGTTCAT |
| | TTGTTACCGATGTATTTGGATCTACTTAACATTTCCAAAGCACGCCGCGTTCT |
| | AATTCTAGATCTGGTAGTCATGTTTGTACACGTCATCCACCTAATACAAATAC |
| | ATATGTCTAGTGTTTGGTGACACTGCCCGTCAGATCTGTTTTTTCCAGATCTG |
| | TGGAACAAATACTCCATGCATGTATGGTAGTTTTGAAACGATCTTGTATCTTC |
| | CATTGTTGTAGTAACAACTAAATAAAGTACAATTGTTCAATTATTGGGAATC |
| | GTATTTTCTGTAGTGCCGATGTACAGCATATTCATAGATGTCTATTTAGGAAC |
| | TCAAATTTTAAATTGAGGACTAGTTATTTATTATGGGTCAGTCTTTTGAATTG |
| | TGTTATCTTGCTGTACTGAAATAATAATGTACCACTAAGGCGCTAACATGTAT |
| | TTTGTCCTTTCAGGCTGACTCGACGGATCCCGGGGGGCAAGAGCTCCTCCCC |
| | CCTCCCCCTCCGCCGCCGCCGGTAACCACCCCGCCCCTCTCCTCTTTCTTTCTC |
| | CGTTTTTTTTTTCGTCTCGGTCTCGATCTTTGGCCTTGGTAGTTTGGGTGGGCG |
| | AGAGCGGCTTCGTCGCCCAGATCGGTGCGCGGGAGGGGCGGGATCTCGCGG |
| | CTGGCGTCTCCGGGCGTGAGTCGGCCCGGATCCTCGCGGGGAATGGGGCTCT |
| | CGGATGTAGATCTTCTTTCTTTCTTCTTTTTGTGGTAGAATTTGAATCCCTCAG |
| | CATTGTTCATCGGTAGTTTTTCTTTTCATGATTTGTGACAAATGCAGCCTCGT |
| | GCGGAGCTTTTTTGTAGGTAGAAGATGGCAGCGAAGATCCGGATCGTCAT |
| | GAAGTCCTTCATGAGCCAGGCCAATAAGGTGGAGGGCGTCATCCCGTAT |
| | GCCCAAAAAGTCGGGCTCCCAGAGTCGCGCTCTCTCTACACAGTCTTGC |
| | GGTCGCCACATATCGATAAAAAGTCCAGAGAACAATTCTCTATGACCGG |
| | CCGCGCCACCATGGGAAAACCTATTCCTAATCCTCTGCTGGGCCTGGAT |
| | TCTACCGGAGGCGTGGACCTGAGAACACTGGGATATTCTCAGCAGCAGC |
| | AGGAGAAGATCAAGCCCAAGGTGAGATCTACAGTGGCCCAGCACCACG |
| | AAGCCCTGGTGGGACACGGATTTACACACGCCCACATTGTGGCCCTGTC |
| | TCAGCACCCTGCCGCCCTGGGAACAGTGGCCGTGAAATATCAGGATATG |
| | ATTGCCGCCCTGCCTGAGGCCACACACGAAGCCATTGTGGGAGTGGGAA |
| | AACGAGGCGCTGGAGCCAGAGCCCTGGAAGCCCTGCTGACAGTGGCCG |
| | GAGAACTGAGAGGACCTCCTCTGCAGCTGGATACAGGACAGCTGCTGAA |
| | GATTGCCAAAAGGGGCGGAGTGACCGCGGTGGAAGCCGTGCACGCCTG |
| | GAGAAATGCCCTGACAGGAGCCCCTCTGAACCTGACCCCCGAACAGGTG |
| | GTGGCCATTGCCAGCAACGGCGGCGGCAAGCAGGCCCTGGAAACCGTG |
| | CAGAGACTGCTGCCCGTGCTGTGCCAGGCCCATGGCCTGACACCTGAAC |
| | AGGTGGTGGCTATCGCCTCTCACGACGGAGGAAAACAGGCTCTGGAAAC |
| | AGTGCAGCGGCTGCTGCCTGTGCTGTGTCAGGCTCACGGCTTGACTCCA |
| | GAACAGGTGGTGGCTATTGCTTCCAACAACGGGGGGAAACAGGCCCTG |
| | GAAACTGTGCAGCGCCTGCTGCCAGTGCTGTGCCAGGCTCACGGACTGA |
| | CCCCCGAACAGGTGGTGGCCATTGCCAGCAACAACGGCGGCAAGCAGG |
| | CCCTGGAAACCGTGCAGAGACTGCTGCCCGTGCTGTGCCAGGCCCATGG |
| | CCTGACACCTGAACAGGTGGTGGCTATCGCCTCTAACGGCGGAGGAAAA |
| | CAGGCTCTGGAAACAGTGCAGCGGCTGCTGCCTGTGCTGTGTCAGGCTC |
| | ACGGCTTGACTCCAGAACAGGTGGTGGCTATTGCTTCCAATATTGGGGG |
| | GAAACAGGCCCTGGAAACTGTGCAGCGCCTGCTGCCAGTGCTGTGCCAG |
| | GCTCACGGGCTGACCCCCGAACAGGTGGTGGCCATTGCCAGCAACATCG |
| | GCGGCAAGCAGGCCCTGGAAACCGTGCAGAGACTGCTGCCCGTGCTGT |
| | GCCAGGCCCATGGCCTGACACCTGAACAGGTGGTGGCTATCGCCTCTAA |
| | CGGCGGAGGAAAACAGGCTCTGGAAACAGTGCAGCGGCTGCTGCCTGT |
| | GCTGTGTCAGGCTCACGGCTTGACTCCAGAACAGGTGGTGGCTATTGCT |
| | TCCCACGACGGGGGGAAACAGGCCCTGGAAACTGTGCAGCGCCTGCTG |
| | CCAGTGCTGTGCCAGGCTCACGGCCTCACTCCCGAACAGGTGGTGGCCA |
| | TTGCCAGCCACGACGGCGGCAAGCAGGCCCTGGAAACCGTGCAGAGAC |
| | TGCTGCCCGTGCTGTGCCAGGCCCATGGCCTGACACCTGAACAGGTGGT |
| | GGCTATCGCCTCTAACAACGGAGGAAAACAGGCTCTGGAAACAGTGCAG |
| | CGGCTGCTGCCTGTGCTGTGTCAGGCTCACGGCTTGACTCCAGAACAGG |
| | TGGTGGCTATTGCTTCCAATATTGGGGGGAAACAGGCCCTGGAAACTGT |
| | GCAGCGCCTGCTGCCAGTGCTGTGCCAGGCTCACGGACTGACCCCCGAA |
| | CAGGTGGTGGCCATTGCCAGCAACATCGGCGGCAAGCAGGCCCTGGAA |
| | ACCGTGCAGAGACTGCTGCCCGTGCTGTGCCAGGCCCATGGCCTGACAC |
| | CTGAACAGGTGGTGGCTATCGCCTCTAACAACGGAGGAAAACAAGCACT |
| | CGAGACAGTGCAGCGGCTGCTGCCTGTGCTGTGTCAGGCTCACGGCTTG |
| | ACTCCAGAACAGGTGGTGGCTATTGCTTCCCACGACGGGGGGAAACAG |
| | GCCCTGGAAACTGTGCAGCGCCTGCTGCCAGTGCTGTGCCAGGCTCACG |
| | GCCTGACCCCCGAACAGGTGGTGGCCATTGCCAGCCACGACGGCGGCA |
| | AGCAGGCCCTGGAAACCGTGCAGAGACTGCTGCCCGTGCTGTGCCAGG |
| | CCCATGGCCTGACACCTGAACAGGTGGTGGCTATCGCCTCTCACGACGG |
| | AGGAAAACAGGCTCTGGAAACAGTGCAGCGGCTGCTGCCTGTGCTGTGT |
| | CAGGCTCACGGCTTGACTCCACAGCAGGTCGTGGCAATTGCTAGCAATA |
| | TCGGCGGACGGCCCGCCCTGGAGAGCATTGTGGCCCAGCTGTCTAGAC |
| | CGGACCCAGCTCTCGCTGCATTGACTAACGACCATCTCGTCGCTTTGGC |
| | TTGCCTCGGTGGAAGGCCAGCTCTTGACGCTGTAAAGAAAGGTTTGCCA |
| | CATGCTCCAGCTCTTATTAAAAGGACTGGTAGCGGTAGCATGAATGAAT |
| | TAACCTTGCACCCTAACGTCTACGCAACAGGAGCCTCCAAGGGTTTGAT |
| | ACACGTATTGGAAAGGGTGTGGGTAAGGGAGCATACTCCAGGTGATGG |
| | AACAATATTTGTCATTAGCGGATTCGGAAATTATAATGGAGGTGTAAGG |
| | TTCTTTGAATCCTTCAGGGAGCATAAACGCAGGGGAGGTGAATTGGTTG |
| | CTGTCTTTGCAGGATCAACCTCGGCTAGGTTGACTTCTAAACAAGTAGT |
| | CCATGAAATGTTGGACTGTGGTGCAGACGTACATATTGTAAATCGCAAA |
| | AGGTTGCTTCATGCAAAATGCTATGGTACGTCCAAAATGAGCGGAGACA |
| | GCTTGATAGTCACAAGCGGTAACTTTACAGGGCCTGGTATGAGCCAAAA |
| | TGTCGAGATGAGCGTGCTCTTGGATGCTGACACGACTGCGGAAATGGGA |
| | TTTAGCTGGGGTACACTCTTGGAAAATCTGTTGGACCAAAGGTGGAATT |
| | ATCACAGACCGACGCTCGGCGACCCTGCGAACCCTGCATGGTCGTTGCT |
| | GTACGACGAACAAGCTGCGGACATACGCCTCGACGAGACAGAGGAATG |
| | AGAATTCATATGAAGATGAAGATGAAATATTTGGTGTGTCAAATAAAAAGCT |
| | TGTGTGCTTAAGTTTGTGTTTTTTTCTTGGCTTGTTGTGTTATGAATTTGTGGC |
| | TTTTTCTAATATTAAATGAATGTAAGATCTCATTATAATGAATAAACAAATGT |
| | TTCTATAATCCATTGTGAATGTTTTGTTGGATCTCTTCTGCAGCATATAACTA |
| | CTGTATGTGCTATGGTATGGACTATGGAATATGATTAAAGATAAG |
| 104 | ATGTTTAAACAAGCTTCCCGTCTTCTGTCACGCTCAGTTGCAGCTGCTTCCTC |
| | TAAGTCCGTCACCACCCGCGCTTTCTCCACCGAGCTGCCATCAACCCTGGACT |
| | CCACCGGCCGCGCCACCATGGGAAAACCGATCCCAAACCCGCTTTTGGGGCT |
| | CGACAGCACAGGAGGGGTTGATCTCAGGACCCTCGGTTACAGCCAACAGCA |
| | ACAAGAAAAAATTAAGCCTAAAGTGAGGTCCACGGTAGCTCAACATCACGA |
| | GGCTTTGGTAGGGCATGGCTTCACGCACGCTCATATCGTCGCTCTCAGCCAA |
| | CATCCCGCTGCGCTTGGGACGGTCGCTGTTAAGTACCAAGACATGATCGCTG |
| | CGCTTCCGGAAGCTACGCATGAGGCGATCGTCGGTGTAGGGAAGCGGGGAG |
| | CAGGTGCGAGGGCGCTCGAGGCGCTCTTGACCGTCGCGGGTGAGCTTAGGGG |
| | TCCGCCACTCCAATTGGACACCGGACAACTTTTGAAAATCGCTAAGAGAGGA |
| | GGCGTCACAGCCGTCGAGGCGGTGCACGCCTGGAGAAATGCCCTGACAGGA |
| | GCCCCTCTGAACCTGACCCCCGAACAGGTGGTGGCCATTGCCAGCAACAACG |
| | GCGGCAAGCAGGCCCTGGAAACCGTGCAGAGACTGCTGCCCGTGCTGTGCCA |
| | GGCCCATGGCCTGACACCTGAACAGGTGGTGGCTATCGCCTCTCACGACGGA |
| | GGAAAACAGGCTCTGGAAACAGTGCAGCGGCTGCTGCCTGTGCTGTGTCAGG |
| | CTCACGGCTTGACTCCAGAACAGGTGGTGGCTATTGCTTCCAATATTGGGGG |
| | GAAACAGGCCCTGGAAACTGTGCAGCGCCTGCTGCCAGTGCTGTGCCAGGCT |
| | CACGGACTGACCCCCGAACAGGTGGTGGCCATTGCCAGCAACATCGGCGGC |
| | AAGCAGGCCCTGGAAACCGTGCAGAGACTGCTGCCCGTGCTGTGCCAGGCCC |
| | ATGGCCTGACACCTGAACAGGTGGTGGCTATCGCCTCTAACAACGGAGGAAA |
| | ACAGGCTCTGGAAACAGTGCAGCGGCTGCTGCCTGTGCTGTGTCAGGCTCAC |
| | GGCTTGACTCCAGAACAGGTGGTGGCTATTGCTTCCAATATTGGGGGGAAAC |
| | AGGCCCTGGAAACTGTGCAGCGCCTGCTGCCAGTGCTGTGCCAGGCTCACGG |
| | GCTGACCCCCGAACAGGTGGTGGCCATTGCCAGCAACGGCGGCGGCAAGCA |
| | GGCCCTGGAAACCGTGCAGAGACTGCTGCCCGTGCTGTGCCAGGCCCATGGC |
| | CTGACACCTGAACAGGTGGTGGCTATCGCCTCTCACGACGGAGGAAAACAG |
| | GCTCTGGAAACAGTGCAGCGGCTGCTGCCTGTGCTGTGTCAGGCTCACGGCT |
| | TGACTCCAGAACAGGTGGTGGCTATTGCTTCCCACGACGGGGGGAAACAGGC |
| | CCTGGAAACTGTGCAGCGCCTGCTGCCAGTGCTGTGCCAGGCTCACGGCCTC |
| | ACTCCCGAACAGGTGGTGGCCATTGCCAGCAACGGCGGCGGCAAGCAGGCC |
| | CTGGAAACCGTGCAGAGACTGCTGCCCGTGCTGTGCCAGGCCCATGGCCTGA |
| | CACCTGAACAGGTGGTGGCTATCGCCTCTCACGACGGAGGAAAACAGGCTCT |
| | GGAAACAGTGCAGCGGCTGCTGCCTGTGCTGTGTCAGGCTCACGGCTTGACT |
| | CCAGAACAGGTGGTGGCTATTGCTTCCAATATTGGGGGGAAACAGGCCCTGG |
| | AAACTGTGCAGCGCCTGCTGCCAGTGCTGTGCCAGGCTCACGGACTGACCCC |
| | CGAACAGGTGGTGGCCATTGCCAGCAACATCGGCGGCAAGCAGGCCCTGGA |
| | AACCGTGCAGAGACTGCTGCCCGTGCTGTGCCAGGCCCATGGCCTGACACCT |
| | GAACAGGTGGTGGCTATCGCCTCTAACAACGGAGGAAAACAAGCACTCGAG |
| | ACAGTGCAGCGGCTGCTGCCTGTGCTGTGTCAGGCTCACGGCTTGACTCCAG |
| | AACAGGTGGTGGCTATTGCTTCCAATATTGGGGGGAAACAGGCCCTGGAAAC |
| | TGTGCAGCGCCTGCTGCCAGTGCTGTGCCAGGCTCACGGCCTGACCCCCGAA |
| | CAGGTGGTGGCCATTGCCAGCAACATCGGCGGCAAGCAGGCCCTGGAAACC |
| | GTGCAGAGACTGCTGCCCGTGCTGTGCCAGGCCCATGGCCTGACACCTGAAC |
| | AGGTGGTGGCTATCGCCTCTAATATCGGAGGAAAACAGGCTCTGGAAACAGT |
| | GCAGCGGCTGCTGCCTGTGCTGTGTCAGGCTCACGGCTTGACTCCACAGCAG |
| | GTCGTGGCAATTGCTAGCAATATCGGCGGACGGCCCGCCCTGGAGAGCATTG |
| | TGGCCCAGCTGTCTAGACCTGATCCTGCCCTGGCCGCCCTGACAAATGATCA |
| | CCTGGTGGCCCTGGCCTGTCTGGGAGGCAGACCTGCCCTGGATGCCGTGAAA |
| | AAAGGACTGCCTCACGCCCCTGCCCTGATCAAGAGAACAGGATCCGGATCCA |
| | TGAACGAGTTAACGCTGCATCCGAATGTGTATGCGACCGGCGCGAGTAAAGG |
| | GCTGATACATGTGCTGGAGCGCGTATGGGTTCGAGAACACACGCCTGGGGAC |
| | GGCACGATCTTCGTGATATCTGGCTTTGGCAACTACAACGGTGGCGTTCGCTT |
| | CTTCGAGTCGTTTCGTGAACACAAGAGACGTGGCGGGGAGCTGGTAGCGGTG |
| | TTCGCTGGCAGCACAAGCGCGCGACTGACAAGCAAACAGGTTGTTCACGAG |
| | ATGCTTGATTGCGGGGCGGATGTTCACATAGTGAACAGAAAGCGACTCCTTC |
| | ACGCTAAGTGTTACGGCACAAGCAAGATGTCAGGTGATTCCCTAATTGTAAC |
| | CTCAGGCAATTTCACAGGTCCGGGAATGTCGCAGAACGTGGAAATGTCTGTA |
| | TTGCTAGACGCAGATACAACCGCCGAGATGGGGTTCTCGTGGGGCACGTTGC |
| | TTGAGAACTTGCTTGATCAGCGATGGAACTACCATCGCCCCACCTTAGGCGA |
| | TCCAGCAAATCCAGCCTGGAGCCTGCTTTATGATGAGCAGGCAGCCGATATT |
| | AGGTTGGATGAAACTGAAGAATGA |
| 105 | MFKQASRLLSRSVAAASSKSVTTRAFSTELPSTLDSTGRATMGKPIPNPLLGLDST |
| | GGVDLRTLGYSQQQQEKIKPKVRSTVAQHHEALVGHGFTHAHIVALSQHPAAL |
| | GTVAVKYQDMIAALPEATHEAIVGVGKRGAGARALEALLTVAGELRGPPLQLD |
| | TGQLLKIAKRGGVTAVEAVHAWRNALTGAPLNLTPEQVVAIASNNGGKQALET |
| | VQRLLPVLCQAHGLTPEQVVAIASHDGGKQALETVQRLLPVLCQAHGLTPEQV |
| | VAIASNIGGKQALETVQRLLPVLCQAHGLTPEQVVAIASNIGGKQALETVQRLLP |
| | VLCQAHGLTPEQVVAIASNNGGKQALETVQRLLPVLCQAHGLTPEQVVAIASNI |
| | GGKQALETVQRLLPVLCQAHGLTPEQVVAIASNGGGKQALETVQRLLPVLCQA |
| | HGLTPEQVVAIASHDGGKQALETVQRLLPVLCQAHGLTPEQVVAIASHDGGKQ |
| | ALETVQRLLPVLCQAHGLTPEQVVAIASNGGGKQALETVQRLLPVLCQAHGLTP |
| | EQVVAIASHDGGKQALETVQRLLPVLCQAHGLTPEQVVAIASNIGGKQALETVQ |
| | RLLPVLCQAHGLTPEQVVAIASNIGGKQALETVQRLLPVLCQAHGLTPEQVVAIA |
| | SNNGGKQALETVQRLLPVLCQAHGLTPEQVVAIASNIGGKQALETVQRLLPVLC |
| | QAHGLTPEQVVAIASNIGGKQALETVQRLLPVLCQAHGLTPEQVVAIASNIGGK |
| | QALETVQRLLPVLCQAHGLTPQQVVAIASNIGGRPALESIVAQLSRPDPALAALT |
| | NDHLVALACLGGRPALDAVKKGLPHAPALIKRTGSGSMNELTLHPNVYATGAS |
| | KGLIHVLERVWVREHTPGDGTIFVISGFGNYNGGVRFFESFREHKRRGGELVAVF |
| | AGSTSARLTSKQVVHEMLDCGADVHIVNRKRLLHAKCYGTSKMSGDSLIVTSG |
| | NFTGPGMSQNVEMSVLLDADTTAEMGFSWGTLLENLLDQRWNYHRPTLGDPA |
| | NPAWSLLYDEQAADIRLDETEE |
| 106 | CTGCAGTGCAGCGTGACCCGGTCGTGCCCCTCTCTAGAGATAATGAGCATTG |
| | CATGTCTAAGTTATAAAAAATTACCACATATTTTTTTTGTCACACTTGTTTGA |
| | AGTGCAGTTTATCTATCTTTATACATATATTTAAACTTTACTCTACGAATAAT |
| | ATAATCTATAGTACTACAATAATATCAGTGTTTTAGAGAATCATATAAATGA |
| | ACAGTTAGACATGGTCTAAAGGACAATTGAGTATTTTGACAACAGGACTCTA |
| | CAGTTTTATCTTTTTAGTGTGCATGTGTTCTCCTTTTTTTTTGCAAATAGCTTC |
| | ACCTATATAATACTTCATCCATTTTATTAGTACATCCATTTAGGGTTTAGGGT |
| | TAATGGTTTTTATAGACTAATTTTTTTAGTACATCTATTTTATTCTATTTTAGC |
| | CTCTAAATTAAGAAAACTAAAACTCTATTTTAGTTTTTTTATTTAATAATTTA |
| | GATATAAAATAGAATAAAATAAAGTGACTAAAAATTAAACAAATACCCTTTA |
| | AGAAATTAAAAAAACTAAGGAAACATTTTTCTTGTTTCGAGTAGATAATGCC |
| | AGCCTGTTAAACGCCGTCGACGAGTCTAACGGACACCAACCAGCGAACCAG |
| | CAGCGTCGCGTCGGGCCAAGCGAAGCAGACGGCACGGCATCTCTGTCGCTGC |
| | CTCTGGACCCCTCTCGAGAGTTCCGCTCCACCGTTGGACTTGCTCCGCTGTCG |
| | GCATCCAGAAATTGCGTGGCGGAGCGGCAGACGTGAGCCGGCACGGCAGGC |
| | GGCCTCCTCCTCCTCTCACGGCACCGGCAGCTACGGGGGATTCCTTTCCCACC |
| | GCTCCTTCGCTTTCCCTTCCTCGCCCGCCGTAATAAATAGACACCCCCTCCAC |
| | ACCCTCTTTCCCCAACCTCGTGTTGTTCGGAGCGCACACACACACAACCAGA |
| | TCTCCCCCAAATCCACCCGTCGGCACCTCCGCTTCAAGGTACGCCGCTCGTCC |
| | TCCCCCCCCCCCCCTCTCTACCTTCTCTAGATCGGCGTTCCGGTCCATGCATG |
| | GTTAGGGCCCGGTAGTTCTACTTCTGTTCATGTTTGTGTTAGATCCGTGTTTGT |
| | GTTAGATCCGTGCTGCTAGCGTTCGTACACGGATGCGACCTGTACGTCAGAC |
| | ACGTTCTGATTGCTAACTTGCCAGTGTTTCTCTTTGGGGAATCCTGGGATGGC |
| | TCTAGCCGTTCCGCAGACGGGATCGATTTCATGATTTTTTTTGTTTCGTTGCAT |
| | AGGGTTTGGTTTGCCCTTTTCCTTTATTTCAATATATGCCGTGCACTTGTTTGT |
| | CGGGTCATCTTTTCATGCTTTTTTTTGTCTTGGTTGTGATGATGTGGTCTGGTT |
| | GGGCGGTCGTTCTAGATCGGAGTAGAATTAATTCTGTTTCAAACTACCTGGT |
| | GGATTTATTAATTTTGGATCTGTATGTGTGTGCCATACATATTCATAGTTACG |
| | AATTGAAGATGATGGATGGAAATATCGATCTAGGATAGGTATACATGTTGAT |
| | GCGGGTTTTACTGATGCATATACAGAGATGCTTTTTGTTCGCTTGGTTGTGAT |
| | GATGTGGTGTGGTTGGGCGGTCGTTCATTCGTTCTAGATCGGAGTAGAATAC |
| | TGTTTCAAACTACCTGGTGTATTTATTAATTTTGGAACTGTATGTGTGTGTCA |
| | TACATCTTCATAGTTACGAGTTTAAGATGGATGGAAATATCGATCTAGGATA |
| | GGTATACATGTTGATGTGGGTTTTACTGATGCATATACATGATGGCATATGCA |
| | GCATCTATTCATATGCTCTAACCTTGAGTACCTATCTATTATAATAAACAAGT |
| | ATGTTTTATAATTATTTTGATCTTGATATACTTGGATGATGGCATATGCAGCA |
| | GCTATATGTGGATTTTTTTAGCCCTGCCTTCATACGCTATTTATTTGCTTGGTA |
| | CTGTTTCTTTTGTCGATGCTCACCCTGTTGTTTGGTGTTACTTCTGCAGGTCGA |
| | CTCTAGAGGATCCATGTTTAAACAAGCTTCCCGTCTTCTGTCACGCTCAG |
| | TTGCAGCTGCTTCCTCTAAGTCCGTCACCACCCGCGCTTTCTCCACCGA |
| | GCTGCCATCAACCCTGGACTCCACCGGCCGCGCCACCATGGGAAAACCG |
| | ATCCCAAACCCGCTTTTGGGGCTCGACAGCACAGGAGGGGTTGATCTCA |
| | GGACCCTCGGTTACAGCCAACAGCAACAAGAAAAAATTAAGCCTAAAGT |
| | GAGGTCCACGGTAGCTCAACATCACGAGGCTTTGGTAGGGCATGGCTTC |
| | ACGCACGCTCATATCGTCGCTCTCAGCCAACATCCCGCTGCGCTTGGGA |
| | CGGTCGCTGTTAAGTACCAAGACATGATCGCTGCGCTTCCGGAAGCTAC |
| | GCATGAGGCGATCGTCGGTGTAGGGAAGCGGGGAGCAGGTGCGAGGGC |
| | GCTCGAGGCGCTCTTGACCGTCGCGGGTGAGCTTAGGGGTCCGCCACTC |
| | CAATTGGACACCGGACAACTTTTGAAAATCGCTAAGAGAGGAGGCGTCA |
| | CAGCCGTCGAGGCGGTGCACGCCTGGAGAAATGCCCTGACAGGAGCCC |
| | CTCTGAACCTGACCCCCGAACAGGTGGTGGCCATTGCCAGCAACAACGG |
| | CGGCAAGCAGGCCCTGGAAACCGTGCAGAGACTGCTGCCCGTGCTGTG |
| | CCAGGCCCATGGCCTGACACCTGAACAGGTGGTGGCTATCGCCTCTCAC |
| | GACGGAGGAAAACAGGCTCTGGAAACAGTGCAGCGGCTGCTGCCTGTG |
| | CTGTGTCAGGCTCACGGCTTGACTCCAGAACAGGTGGTGGCTATTGCTT |
| | CCAATATTGGGGGGAAACAGGCCCTGGAAACTGTGCAGCGCCTGCTGCC |
| | AGTGCTGTGCCAGGCTCACGGACTGACCCCCGAACAGGTGGTGGCCATT |
| | GCCAGCAACATCGGCGGCAAGCAGGCCCTGGAAACCGTGCAGAGACTG |
| | CTGCCCGTGCTGTGCCAGGCCCATGGCCTGACACCTGAACAGGTGGTGG |
| | CTATCGCCTCTAACAACGGAGGAAAACAGGCTCTGGAAACAGTGCAGCG |
| | GCTGCTGCCTGTGCTGTGTCAGGCTCACGGCTTGACTCCAGAACAGGTG |
| | GTGGCTATTGCTTCCAATATTGGGGGGAAACAGGCCCTGGAAACTGTGC |
| | AGCGCCTGCTGCCAGTGCTGTGCCAGGCTCACGGGCTGACCCCCGAACA |
| | GGTGGTGGCCATTGCCAGCAACGGCGGCGGCAAGCAGGCCCTGGAAAC |
| | CGTGCAGAGACTGCTGCCCGTGCTGTGCCAGGCCCATGGCCTGACACCT |
| | GAACAGGTGGTGGCTATCGCCTCTCACGACGGAGGAAAACAGGCTCTG |
| | GAAACAGTGCAGCGGCTGCTGCCTGTGCTGTGTCAGGCTCACGGCTTGA |
| | CTCCAGAACAGGTGGTGGCTATTGCTTCCCACGACGGGGGGAAACAGG |
| | CCCTGGAAACTGTGCAGCGCCTGCTGCCAGTGCTGTGCCAGGCTCACGG |
| | CCTCACTCCCGAACAGGTGGTGGCCATTGCCAGCAACGGCGGCGGCAA |
| | GCAGGCCCTGGAAACCGTGCAGAGACTGCTGCCCGTGCTGTGCCAGGC |
| | CCATGGCCTGACACCTGAACAGGTGGTGGCTATCGCCTCTCACGACGGA |
| | GGAAAACAGGCTCTGGAAACAGTGCAGCGGCTGCTGCCTGTGCTGTGTC |
| | AGGCTCACGGCTTGACTCCAGAACAGGTGGTGGCTATTGCTTCCAATAT |
| | TGGGGGGAAACAGGCCCTGGAAACTGTGCAGCGCCTGCTGCCAGTGCT |
| | GTGCCAGGCTCACGGACTGACCCCCGAACAGGTGGTGGCCATTGCCAG |
| | CAACATCGGCGGCAAGCAGGCCCTGGAAACCGTGCAGAGACTGCTGCC |
| | CGTGCTGTGCCAGGCCCATGGCCTGACACCTGAACAGGTGGTGGCTATC |
| | GCCTCTAACAACGGAGGAAAACAAGCACTCGAGACAGTGCAGCGGCTG |
| | CTGCCTGTGCTGTGTCAGGCTCACGGCTTGACTCCAGAACAGGTGGTGG |
| | CTATTGCTTCCAATATTGGGGGGAAACAGGCCCTGGAAACTGTGCAGCG |
| | CCTGCTGCCAGTGCTGTGCCAGGCTCACGGCCTGACCCCCGAACAGGTG |
| | GTGGCCATTGCCAGCAACATCGGCGGCAAGCAGGCCCTGGAAACCGTG |
| | CAGAGACTGCTGCCCGTGCTGTGCCAGGCCCATGGCCTGACACCTGAAC |
| | AGGTGGTGGCTATCGCCTCTAATATCGGAGGAAAACAGGCTCTGGAAAC |
| | AGTGCAGCGGCTGCTGCCTGTGCTGTGTCAGGCTCACGGCTTGACTCCA |
| | CAGCAGGTCGTGGCAATTGCTAGCAATATCGGCGGACGGCCCGCCCTG |
| | GAGAGCATTGTGGCCCAGCTGTCTAGACCTGATCCTGCCCTGGCCGCCC |
| | TGACAAATGATCACCTGGTGGCCCTGGCCTGTCTGGGAGGCAGACCTGC |
| | CCTGGATGCCGTGAAAAAAGGACTGCCTCACGCCCCTGCCCTGATCAAG |
| | AGAACAGGATCCGGATCCATGAACGAGTTAACGCTGCATCCGAATGTGT |
| | ATGCGACCGGCGCGAGTAAAGGGCTGATACATGTGCTGGAGCGCGTAT |
| | GGGTTCGAGAACACACGCCTGGGGACGGCACGATCTTCGTGATATCTGG |
| | CTTTGGCAACTACAACGGTGGCGTTCGCTTCTTCGAGTCGTTTCGTGAA |
| | CACAAGAGACGTGGCGGGGAGCTGGTAGCGGTGTTCGCTGGCAGCACA |
| | AGCGCGCGACTGACAAGCAAACAGGTTGTTCACGAGATGCTTGATTGCG |
| | GGGCGGATGTTCACATAGTGAACAGAAAGCGACTCCTTCACGCTAAGTG |
| | TTACGGCACAAGCAAGATGTCAGGTGATTCCCTAATTGTAACCTCAGGC |
| | AATTTCACAGGTCCGGGAATGTCGCAGAACGTGGAAATGTCTGTATTGC |
| | TAGACGCAGATACAACCGCCGAGATGGGGTTCTCGTGGGGCACGTTGCT |
| | TGAGAACTTGCTTGATCAGCGATGGAACTACCATCGCCCCACCTTAGGC |
| | GATCCAGCAAATCCAGCCTGGAGCCTGCTTTATGATGAGCAGGCAGCCG |
| | ATATTAGGTTGGATGAAACTGAAGAATGAGAATTCGGTACCGAGTTTGAA |
| | TCAAATCTTCACTTGTTTAATGAGATATGCGAGACGCCTATGATCGCATGATA |
| | TTTGCTTTCAATTCTGTTGTGCACGTTGTAAAAAACCTGAGCATGTGTAGCTC |
| | AGATCCTTACCGCCGGTTTCGGTTCATTCTAATGAATATATCACCCGTTACTA |
| | TCGTATTTTTATGAATAATATTCTCCGTTCAATTTACTGATTGTACCCTACTAC |
| | TTATATGTACAATATTAAAATGAAAACAATATATTGTGCTGAATAGGTTTAT |
| | AGCGACATCTATGATAGAGCGCCACAATAACAAACAATTGCGTTTTATTATT |
| | ACAAATCCAATTTTAAAAAAAGCGGCAGAACCGGTCAAACCTAAAAGACTG |
| | ATTACATAAATCTTATTCAAATTTCAAAAGTGCCCCAGGGGCTAGTATCTAC |
| | GACACACCGAGCGGCGAATTCAGTACATTAAAAACGTCCGCAATGTGTTATT |
| | AAGTTGTCTAAGCGTCAATTTGTTTACACCACAATATATCCTGCCACCAGCCA |
| | GCCAACAGCTCCCCGACCGGCAGCTCGGCACAAAATCACCACTCGATACAGG |
| | CAGCCCATCAGTCCGGGACGGCGTCAGCGGGA |
| 107 | AAGTCGAGGTATTTCGAATTGTTTGTTGACACGGACAAAGTAAGGGAAAACC |
| | CCTGAAAGAATTTCCATATTGACCTTGGACATATAAGAGTTCCGAATCGAAT |
| | CTCTTTAGAAAGAAGATCTTTTGTCTCATGGTAGCCTGCTCCAGTCCCCTTAC |
| | GAAACTTTCGTTATTGGGTTAGCCATACACTTCACATGTTTCTAGCGATTCAC |
| | ATGGCATCATCAAATGATACAAGTCTTGGATAAGAATCTACAACGCACTAGA |
| | ACGCCCTTGTTGACGATTCTTTACTGCGACAGCATCTAGGGCTCCTCGAATAA |
| | TGCGATATCTCACACCGGGTAAATCCTTAACCCTTCCTCCTCTTACTAATACT |
| | ACAGAATGTTCTTGTAAATTATGGCCAATACCAGGTATATAAGCAGTGATTT |
| | CAAATCCAGAGGTTAATCGTACTCTGGCAACTTTACGTAAGGCAGAGTTGGG |
| | TTTTTTGGGGTTGATAGTGGAAAAGTCGACAGATAAGTCACCCTTCCTGTCCC |
| | TCTACAGAATCGTACATGAGATTTTCACCTAATACGGCTCCTCGTTCAATTCT |
| | TTCGAAGGGATCCTTTTCCTCGTTCGAGAGTCTTCGCCCTTTCTTCCACTCCGT |
| | CCCGAAGACTAACTAAGACCAATTGAGTCACGTTTTCATGTTCTAATTGAAC |
| | ACTTTCCATTTATGATTAAAGGAGAAGATTGTTCTTTTACCAAACATATGCGG |
| | ATCAAATCACGTCTTATAATAAGAAGAAATCTTTCTCGGTATCAATCCCCTTG |
| | CCCCTCATTCTTTGAGAATCAGAAGGATCCTTTTCGAGTTTCCATTTCTTCATT |
| | TGGAATCTGGGCTCTTCTATCTTCGACTTATTTTTTTTTTGCTTTATTCTTTATT |
| | TATTTCATTTCGATTTTTCCCTCTTCCTCTATCCCTATCCTCTAGGTACAGCGT |
| | TTGCATCAATAGAGAACCTTTTCCTCTGTATGAATCTATATTATTCCATTCCA |
| | ATTTCTTCCCGAAACTTCCCAAGAAAAATCCCGAATTGGATCCAAAATTGAC |
| | GGGTTAATGTGAGCTTATCCATGCGGTTAGGCACTCTTCAAATAGGAATCCA |
| | TTTTCTAACTGGCTTTCGTGCTTTGGTGAGTCGTCCGAGATCCTTTCGATGAC |
| | CTATGTTGTGTTGAAGGGATATCTATATGATCCGATCGATTGCATAAGACCC |
| | GCGGTAGCAATAGAACGGGGAAAGTATACAGAAAAGACAGTTCTTTTCAATT |
| | TCGATTATCTATATATTAGTTGTTTCTATTTCTAGATATCTATTTCTATATATT |
| | AGTATTAGTTAGTAGTACTATTCTATTAGTTAGCGATCCCGGCTCTGTGAGTT |
| | CTTTCTTCCGTGATGAACTGTCGGCACCAGTCCTCCATTTTTTCTCTGTGGACC |
| | GAGGAGAAAGGGGGCTCAGCAGGAAGAGGATTGTACCATGAGAGAAGCAC |
| | AGAGGTCAACCCTCTTCAAATATGGAACATGGATTCTGCAATGCAACGTAGT |
| | TGGGTCCTCATATCGATCCGAATGAATCATTCTTTCTACAGAGGTCAATCTTT |
| | GCCTATTAGGCAAGAGGATAGCAAGTTCGAAATTCTGTCTCGGTAGGACATG |
| | GATTTCTATTACTATGAAATTCATAAATGAAATGAAGTAGTTAATGGGAGGG |
| | CTACCATTATCCTTTTTCTTGTATGTGTTCCTAAGAGAAGGAATTTGTCCATTT |
| | CATGTTTCGAGGTCTCAAAAAAGGGCGTGGAAACAGATAAGAAACTCTTGA |
| | ATGGAAATTGAAAAGAAATGTAGCCCCAGTTCCTTCGGAAATGGTAAGATCT |
| | TTGGCGCAAGAAGAAGGGGCGACCCATATCATCTTGACTTGGTTCTGCTTCC |
| | CCTCTTTTTTTAAGAATACCGAGTCGGGTTCTTCTCCTACCAGTATCGAATAG |
| | AACATGCTGAACAAGATCTTCTTCATGGAAACCTGCTCGATTTAGATCGGGA |
| | CTAGTTTCTTTGAGCTCAACCCAATGTGAGTTTTTTCTATTTTGACTTACTCCC |
| | CCCGCCACGATCGAACGGGAATGGATAAGAGGCTTGTGGGATTGACGTGAT |
| | AGGGTAGGGTTGGCTATACTGCTGGTGGTAATACGTGTGCTTGGGAGTCCTT |
| | GCAATTTGAATAAACCAAGATCTTACCATGAAGACCGGCCGTAAGAACCA |
| | TCAACGACATCATGTCCTTCCCGCACGAGGCCGAGTGGGGGCGGCGGC |
| | GGTCAGGTGCTCGGCGGTGTCCCCAGTCACCCCACCATCCCCAGCGCCA |
| | CCAGCCACTCCACTCCGACCATGGGGGCCAGCCGAGCCCCGTAAGGGC |
| | GCGGACATCCTCGTGGAGGCGCTGGAGCGATGCGGCGTCAGCGACGTG |
| | TTCGCCTATCCAGGCGGCGCGTCCATGGAGATCCATCAAGCGCTGACTC |
| | GTTCCCCAGTCATCACCAACCATCTCTTCCGTCATGAGCAAGGCGAGGC |
| | GTTCGCGGCGTCCGGGTATGCGCGTGCGTCCGGCCGTGTCGGGGTCTG |
| | CGTCGCCACCTCCGGCCCCGGGGCAACCAACCTCGTGTCCGCGCTCGCC |
| | GACGCGCTGCTCGACTCCGTCCCAATGGTCGCCATCACTGGCCAAGTCC |
| | CCCGTCGTATGATCGGCACCGACGCCTTCCAAGAGACTCCCATAGTCGA |
| | GGTCACCCGTTCCATCACCAAGCATAATTATCTTGTCCTTGATGTGGAG |
| | GACATCCCCCGTGTCATACAAGAAGCCTTCTTCCTCGCGTCCTCGGGCC |
| | GTCCTGGCCCAGTGCTGGTCGACATCCCCAAGGACATCCAACAACAAAT |
| | GGCCGTGCCAGTCTGGGACACCTCGATGAATCTACCAGGGTATATCGCA |
| | CGTCTGCCCAAGCCACCCGCGACAGAATTGCTTGAGCAAGTCTTGCGTC |
| | TGGTTGGCGAGTCACGACGTCCAATTCTCTATGTCGGTGGTGGCTGCTC |
| | TGCATCTGGTGACGAATTGCGTTGGTTTGTTGAGCTGACTGGTATCCCA |
| | GTTACAACCACTCTGATGGGCCTCGGCAATTTCCCCAGTGACGACCCAT |
| | TGTCCCTGCGTATGCTTGGGATGCATGGCACTGTGTATGCAAATTATGC |
| | CGTGGATAAGGCTGACCTGTTGCTTGCGTTTGGTGTGCGATTTGATGAT |
| | CGTGTGACAGGGAAAATTGAGGCTTTTGCAAGCAGGGCCAAGATTGTGC |
| | ATATTGACATTGATCCAGCAGAGATTGGAAAGAACAAGCAACCACATGT |
| | GTCAATTTGCGCAGATGTTAAACTTGCTTTACAAGGCTTGAATGCTCTGC |
| | TACAACAAAGCACAACAAAGACAAGTTCTGATTTTAGTGCATGGCATAA |
| | TGAGTTGGACCAACAAAAGAGGGAGTTTCCTCTGGGGTATAAAACTTTT |
| | GGTGAAGAGATCCCACCACAATATGCCATTCAAGTGCTGGATGAGCTGA |
| | CTAAAGGTGAGGCAATCATCGCTACTGGTGTTGGGCAACATCAAATGTG |
| | GGCGGCACAATATTATACCTATAAGCGACCACGACAATGGCTGTCTTCG |
| | GCTGGTCTGGGCGCAATGGGATTTGGGCTGCCTGCTGCAGCTGGTGCTT |
| | CTGTGGCTAACCCAGGTGTCACAGTTGTTGATATTGATGGGGATGGTAG |
| | CTTCCTCATGAACATTCAAGAGCTGGCATTGATCCGTATTGAGAACCTCC |
| | CTGTGAAGGTGATGGTGTTGAACAACCAACATTTGGGTATGGTGGTGCA |
| | ATTGGAGGATAGGTTTTATAAGGCGAATAGGGCGCATACATATTTGGGC |
| | AACCCAGAATGTGAGAGCGAGATATATCCAGATTTTGTGACTATTGCTA |
| | AGGGGTTCAATATTCCTGCAGTCCGTGTAACAAAGAAGAGTGAAGTCCG |
| | TGCCGCCATCAAGAAGATGCTTGAGACTCCAGGGCCATATTTGTTGGAT |
| | ATCATCGTCCCACATCAAGAGCATGTGCTGCCTATGATCCCAATTGGGG |
| | GCGCATTCAAGGACATGATCCTGGATGGTGATGGCAGGACTGTGTATTA |
| | AGGTACCTTTTTCTCTAAGCTTGGCTTTTCTGCTAACATATAGCAATTTTTGA |
| | AGAAAGGAAAGCTAGAAATACCCAATATCTTGCTGAAGCAAGATATTGGGT |
| | ATTTCTTTTTTTTTTATTTTGAATCTTTCTATTCTGAATTGGCGCCAAGGGTGG |
| | CCTTGAGTTTCTCGCCCCTTTGCCTTAGGATTCGGTAATTCTCTTTCTCGAGGG |
| | GACGGGGAAGGGATATAACTCAGCGGTAGAGTGTCACCTTGACGTGGTGGA |
| | AGTCATCAGTTCGAGCCTGATTATCCCTAAACCCAATGTGAGTTTTTTCTATT |
| | TTGACTTACTCCCCCCGCCACGATCGAACGGGAATGGATAAGAGGCTTGTGG |
| | GATTGACGTGATAGGGTAGGGTTGGCTATACTGCTGGTGGCGAACTCCAGGC |
| | TAATAATCTGAAGCGCATGGATACAAGTTATCCTTGGAAGGAAAGACAATTC |
| | CGAATCCGCTTTGTCTACGAATAAGGAAGCTATAAGTAATGCAACTATGAAT |
| | CTCATGGAGAGTTCGATCCTGGCTCAGGATGAACGCTGGCGGCATGCTTAAC |
| | ACATGCAAGTCGAACGGGAAGTGGTGTTTCCAGTGGCGAACGGGTGAGTAA |
| | CGCGTAAGAACCTGCCCTTGGGAGGGGAACAACAACTGGAAACGGTTGCTA |
| | ATACCCCGTAGGCTGAGGAGCAAAAGGAGAAATCCGCCCAAGGAGGGGCTC |
| | GCGTCTGATTAGCTAGTTGGTGAGGCAATAGCTTACCAAGGCGATGATCAGT |
| | AGCTGGTCCGAGAGGATGATCAGCCACACTGGGACTGAGACACGGCCCAGA |
| | CTCCTACGGGAGGCAGCAGTGGGGAATTTTCCGCAATGGGCGAAAGCCTGAC |
| | GGAGCAATGCCGCGTGGAGGTGGAAGGCCCACGGGTCGTCAACTTCTTTTCT |
| | CGGAGAAGAAACAATGACGGTATCTGAGGAATAAGCATCGGCTAACTCTGT |
| | GCCAGCAGCCGCGGTAAGACAGAGGATGCAAGCGTTATCCGGAATGATTGG |
| | GCGTAAAGCGTCTGTAGGTGGCTTTTCAAGTCCGCCGTCAAATCCCAGGGCT |
| | CAACCCTGGACAGGCGGTGGAAACTACCAAGCTGGAGTACGGTAGGGGCAG |
| | AGGGAATTTCCGGTGGAGCGGTGAAATGCATTGAGATCGGAAAGAACACCA |
| | ACGGCGAAAGCACTCTGCTGGGCCGACACTGACACTGAGAGACGAAAGCTA |
| | GGGGAGCAAATGGGATTAGAGACCCCAGTAGTCCTAGCCGTAAACGATGGA |
| | TACTAGGTGCTGTGCGACTCGACCCGTGCAGTGCTGTAGCTAACGCGTTAAG |
| | TATCCCGCCTGGGGAGTACGTTCGCAAGAATGAAACTCAAAGGAATTGACGG |
| | GGGCCCGCACAAGCGGTGGAGCATGTGGTTTAATTCGATGCAAAGCGAAGA |
| | ACCTTACCAGGGCTTGACATGCCGCGAATCCTCTTGAAAGAGAGGGGTGCCC |
| | TCGGGAACGCGGACACAGGTGGTGCATGGCTGTCGTCAGCTCGTGCCGTAAG |
| | GTGTTGGGTTAAGTCTCGCAACGAGCGCAACCCTCGTGTTTAGTTGCCACTAT |
| | GAGTTTGGAACCCTGAACAGACCGCCGGTGTTAAGCCGGAGGAAGGAGAGG |
| | ATGAGGCCAAGTCATCATGCCCCTTATGCCCTGGGCGACACACGTGCTACAA |
| | TGGGCGGGACAAAGGGTCGCGATCTCGCGAGGGTGAGCTAACTCCAAAAAC |
| | CCGTCCTCAGTTCGGATTGCAGGCTGCAACTCGCCTGCATGAAGCAGGAATC |
| | GCTAGTAATCGCCGGTCAGCCATACGGCGGTGAATCCGTTCCCGGGCCTTGT |
| | ACACACCGCCCGTCACACTATAGGAGCTGGCCATGTTTGAAGTCATTACCCT |
| | TAACCGTAAGGAGGGGGATGCCTAAGGCTAGGCTTGCGACTGGAGTGAAGT |
| | CGTAACAAGGTAGCCGTACTGGAAGGTGCGGCTGGATCACCTCCTTTTCAGG |
| | GAGAGCTAATGCTTATGCTTATTGGGTATTTTGGTTTGACACTGCTTCACGCC |
| | CAAAAAGAAGGCAGCTACGTCTGAGCTAAACTTGGATATGGAAGTCTTCTTT |
| | CGTTTAGGGTGAAGTAAGACCAAGCT |
| 108 | AAGTCGAGGTATTTCGAATTGTTTGTTGACACGGACAAAGTAAGGGAAAACC |
| | CCTGAAAGAATTTCCATATTGACCTTGGACATATAAGAGTTCCGAATCGAAT |
| | CTCTTTAGAAAGAAGATCTTTTGTCTCATGGTAGCCTGCTCCAGTCCCCTTAC |
| | GAAACTTTCGTTATTGGGTTAGCCATACACTTCACATGTTTCTAGCGATTCAC |
| | ATGGCATCATCAAATGATACAAGTCTTGGATAAGAATCTACAACGCACTAGA |
| | ACGCCCTTGTTGACGATTCTTTACTGCGACAGCATCTAGGGCTCCTCGAATAA |
| | TGCGATATCTCACACCGGGTAAATCCTTAACCCTTCCTCCTCTTACTAATACT |
| | ACAGAATGTTCTTGTAAATTATGGCCAATACCAGGTATATAAGCAGTGATTT |
| | CAAATCCAGAGGTTAATCGTACTCTGGCAACTTTACGTAAGGCAGAGTTGGG |
| | TTTTTTGGGGTTGATAGTGGAAAAGTCGACAGATAAGTCACCCTTCCTGTCCC |
| | TCTACAGAATCGTACATGAGATTTTCACCTAATACGGCTCCTCGTTCAATTCT |
| | TTCGAAGGGATCCTTTTCCTCGTTCGAGAGTCTTCGCCCTTTCTTCCACTCCGT |
| | CCCGAAGACTAACTAAGACCAATTGAGTCACGTTTTCATGTTCTAATTGAAC |
| | ACTTTCCATTTATGATTAAAGGAGAAGATTGTTCTTTTACCAAACATATGCGG |
| | ATCAAATCACGTCTTATAATAAGAAGAAATCTTTCTCGGTATCAATCCCCTTG |
| | CCCCTCATTCTTTGAGAATCAGAAGGATCCTTTTCGAGTTTCCATTTCTTCATT |
| | TGGAATCTGGGCTCTTCTATCTTCGACTTATTTTTTTTTTGCTTTATTCTTTATT |
| | TATTTCATTTCGATTTTTCCCTCTTCCTCTATCCCTATCCTCTAGGTACAGCGT |
| | TTGCATCAATAGAGAACCTTTTCCTCTGTATGAATCTATATTATTCCATTCCA |
| | ATTTCTTCCCGAAACTTCCCAAGAAAAATCCCGAATTGGATCCAAAATTGAC |
| | GGGTTAATGTGAGCTTATCCATGCGGTTAGGCACTCTTCAAATAGGAATCCA |
| | TTTTCTAACTGGCTTTCGTGCTTTGGTGAGTCGTCCGAGATCCTTTCGATGAC |
| | CTATGTTGTGTTGAAGGGATATCTATATGATCCGATCGATTGCATAAGACCC |
| | GCGGTAGCAATAGAACGGGGAAAGTATACAGAAAAGACAGTTCTTTTCAATT |
| | TCGATTATCTATATATTAGTTGTTTCTATTTCTAGATATCTATTTCTATATATT |
| | AGTATTAGTTAGTAGTACTATTCTATTAGTTAGCGATCCCGGCTCTGTGAGTT |
| | CTTTCTTCCGTGATGAACTGTCGGCACCAGTCCTCCATTTTTTCTCTGTGGACC |
| | GAGGAGAAAGGGGGCTCAGCAGGAAGAGGATTGTACCATGAGAGAAGCAC |
| | AGAGGTCAACCCTCTTCAAATATGGAACATGGATTCTGCAATGCAACGTAGT |
| | TGGGTCCTCATATCGATCCGAATGAATCATTCTTTCTACAGAGGTCAATCTTT |
| | GCCTATTAGGCAAGAGGATAGCAAGTTCGAAATTCTGTCTCGGTAGGACATG |
| | GATTTCTATTACTATGAAATTCATAAATGAAATGAAGTAGTTAATGGGAGGG |
| | CTACCATTATCCTTTTTCTTGTATGTGTTCCTAAGAGAAGGAATTTGTCCATTT |
| | CATGTTTCGAGGTCTCAAAAAAGGGCGTGGAAACAGATAAGAAACTCTTGA |
| | ATGGAAATTGAAAAGAAATGTAGCCCCAGTTCCTTCGGAAATGGTAAGATCT |
| | TTGGCGCAAGAAGAAGGGGCGACCCATATCATCTTGACTTGGTTCTGCTTCC |
| | CCTCTTTTTTTAAGAATACCGAGTCGGGTTCTTCTCCTACCAGTATCGAATAG |
| | AACATGCTGAACAAGATCTTCTTCATGGAAACCTGCTCGATTTAGATCGGG |
| 109 | AAGGGTGGCCTTGAGTTTCTCGCCCCTTTGCCTTAGGATTCGGTAATTCTCTT |
| | TCTCGAGGGGACGGGGAAGGGATATAACTCAGCGGTAGAGTGTCACCTTGA |
| | CGTGGTGGAAGTCATCAGTTCGAGCCTGATTATCCCTAAACCCAATGTGAGT |
| | TTTTTCTATTTTGACTTACTCCCCCCGCCACGATCGAACGGGAATGGATAAGA |
| | GGCTTGTGGGATTGACGTGATAGGGTAGGGTTGGCTATACTGCTGGTGGCGA |
| | ACTCCAGGCTAATAATCTGAAGCGCATGGATACAAGTTATCCTTGGAAGGAA |
| | AGACAATTCCGAATCCGCTTTGTCTACGAATAAGGAAGCTATAAGTAATGCA |
| | ACTATGAATCTCATGGAGAGTTCGATCCTGGCTCAGGATGAACGCTGGCGGC |
| | ATGCTTAACACATGCAAGTCGAACGGGAAGTGGTGTTTCCAGTGGCGAACGG |
| | GTGAGTAACGCGTAAGAACCTGCCCTTGGGAGGGGAACAACAACTGGAAAC |
| | GGTTGCTAATACCCCGTAGGCTGAGGAGCAAAAGGAGAAATCCGCCCAAGG |
| | AGGGGCTCGCGTCTGATTAGCTAGTTGGTGAGGCAATAGCTTACCAAGGCGA |
| | TGATCAGTAGCTGGTCCGAGAGGATGATCAGCCACACTGGGACTGAGACAC |
| | GGCCCAGACTCCTACGGGAGGCAGCAGTGGGGAATTTTCCGCAATGGGCGA |
| | AAGCCTGACGGAGCAATGCCGCGTGGAGGTGGAAGGCCCACGGGTCGTCAA |
| | CTTCTTTTCTCGGAGAAGAAACAATGACGGTATCTGAGGAATAAGCATCGGC |
| | TAACTCTGTGCCAGCAGCCGCGGTAAGACAGAGGATGCAAGCGTTATCCGGA |
| | ATGATTGGGCGTAAAGCGTCTGTAGGTGGCTTTTCAAGTCCGCCGTCAAATC |
| | CCAGGGCTCAACCCTGGACAGGCGGTGGAAACTACCAAGCTGGAGTACGGT |
| | AGGGGCAGAGGGAATTTCCGGTGGAGCGGTGAAATGCATTGAGATCGGAAA |
| | GAACACCAACGGCGAAAGCACTCTGCTGGGCCGACACTGACACTGAGAGAC |
| | GAAAGCTAGGGGAGCAAATGGGATTAGAGACCCCAGTAGTCCTAGCCGTAA |
| | ACGATGGATACTAGGTGCTGTGCGACTCGACCCGTGCAGTGCTGTAGCTAAC |
| | GCGTTAAGTATCCCGCCTGGGGAGTACGTTCGCAAGAATGAAACTCAAAGGA |
| | ATTGACGGGGGCCCGCACAAGCGGTGGAGCATGTGGTTTAATTCGATGCAAA |
| | GCGAAGAACCTTACCAGGGCTTGACATGCCGCGAATCCTCTTGAAAGAGAGG |
| | GGTGCCCTCGGGAACGCGGACACAGGTGGTGCATGGCTGTCGTCAGCTCGTG |
| | CCGTAAGGTGTTGGGTTAAGTCTCGCAACGAGCGCAACCCTCGTGTTTAGTT |
| | GCCACTATGAGTTTGGAACCCTGAACAGACCGCCGGTGTTAAGCCGGAGGAA |
| | GGAGAGGATGAGGCCAAGTCATCATGCCCCTTATGCCCTGGGCGACACACGT |
| | GCTACAATGGGCGGGACAAAGGGTCGCGATCTCGCGAGGGTGAGCTAACTC |
| | CAAAAACCCGTCCTCAGTTCGGATTGCAGGCTGCAACTCGCCTGCATGAAGC |
| | AGGAATCGCTAGTAATCGCCGGTCAGCCATACGGCGGTGAATCCGTTCCCGG |
| | GCCTTGTACACACCGCCCGTCACACTATAGGAGCTGGCCATGTTTGAAGTCA |
| | TTACCCTTAACCGTAAGGAGGGGGATGCCTAAGGCTAGGCTTGCGACTGGAG |
| | TGAAGTCGTAACAAGGTAGCCGTACTGGAAGGTGCGGCTGGATCACCTCCTT |
| | TTCAGGGAGAGCTAATGCTTATGCTTATTGGGTATTTTGGTTTGACACTGCTT |
| | CACGCCCAAAAAGAAGGCAGCTACGTCTGAGCTAAACTTGGATATGGAAGT |
| | CTTCTTTCGTTTAGGGTGAAGTAAGACCAAGCT |
| 110 | TCACATTGGGTTGAGCTCAAAGAAACTAGT |
| 111 | CATTGCTATTTGACTCGGACCTGTTCTG |
| 112 | TGTTCCGACCTAGGATAATAAGCTCATG |
| 113 | ATGGTGGTGCAATTGGAGGATAGGTTTTAT |
| 114 | CAATTGTCAACCGGTCCT |
| 115 | ATGGCTCGAATCCATAGT |
| 116 | ATGGCCCCCTCCGTGATGGCGTCGTCGGCCACCACCGTCGCTCCCTTCCAGG |
| | GCTCAAGTCCACCGCCGGCTTGCCGTCGCCCGCCGTCCGAACTCCAGCTTCG |
| | GCAACGTCAGCATGGCGGCAGGATCAGATGCACCGGCCGCGCCACCATGGG |
| | AAAACCTATTCCTAATCCTCTGCTGGGCCTGGATTCTACCGGAGGCGTGGAC |
| | CTGAGAACACTGGGATATTCTCAGCAGCAGCAGGAGAAGATCAAGCCCAAG |
| | GTGAGATCTACAGTGGCCCAGCACCACGAAGCCCTGGTGGGACACGGATTTA |
| | CACACGCCCACATTGTGGCCCTGTCTCAGCACCCTGCCGCCCTGGGAACAGT |
| | GGCCGTGAAATATCAGGATATGATTGCCGCCCTGCCTGAGGCCACACACGAA |
| | GCCATTGTGGGAGTGGGAAAACGAGGCGCTGGAGCCAGAGCCCTGGAAGCC |
| | CTGCTGACAGTGGCCGGAGAACTGAGAGGACCTCCTCTGCAGCTGGATACAG |
| | GACAGCTGCTGAAGATTGCCAAAAGGGGCGGAGTGACCGCGGTGGAAGCCG |
| | TGCACACCTGGAGAAATGCCCTGACAGGAGCCCCTCTGAACCTGACCCCCGA |
| | ACAGGTGGTGGCCATTGCCAGCCACGACGGCGGCAAGCAGGCCCTGGAAAC |
| | CGTGCAGAGACTGCTGCCCGTGCTGTGCCAGGCCCATGGCCTGACACCTGAA |
| | CAGGTGGTGGCTATCGCCTCTAATATCGGAGGAAAACAGGCTCTGGAAACAG |
| | TGCAGCGGCTGCTGCCTGTGCTGTGTCAGGCTCACGGCTTGACTCCAGAACA |
| | GGTGGTGGCTATTGCTTCCAATATTGGGGGGAAACAGGCCCTGGAAACTGTG |
| | CAGCGCCTGCTGCCAGTGCTGTGCCAGGCTCACGGACTGACCCCCGAACAGG |
| | TGGTGGCCATTGCCAGCAACGGCGGCGGCAAGCAGGCCCTGGAAACCGTGC |
| | AGAGACTGCTGCCCGTGCTGTGCCAGGCCCATGGCCTGACACCTGAACAGGT |
| | GGTGGCTATCGCCTCTAACGGCGGAGGAAAACAGGCTCTGGAAACAGTGCA |
| | GCGGCTGCTGCCTGTGCTGTGTCAGGCTCACGGCTTGACTCCAGAACAGGTG |
| | GTGGCTATTGCTTCCAACAACGGGGGGAAACAGGCCCTGGAAACTGTGCAGC |
| | GCCTGCTGCCAGTGCTGTGCCAGGCTCACGGGCTGACCCCCGAACAGGTGGT |
| | GGCCATTGCCAGCAACGGCGGCGGCAAGCAGGCCCTGGAAACCGTGCAGAG |
| | ACTGCTGCCCGTGCTGTGCCAGGCCCATGGCCTGACACCTGAACAGGTGGTG |
| | GCTATCGCCTCTCACGACGGAGGAAAACAGGCTCTGGAAACAGTGCAGCGG |
| | CTGCTGCCTGTGCTGTGTCAGGCTCACGGCTTGACTCCAGAACAGGTGGTGG |
| | CTATTGCTTCCAACATCGGGGGGAAACAGGCCCTGGAAACTGTGCAGCGCCT |
| | GCTGCCAGTGCTGTGCCAGGCTCACGGCCTCACTCCCGAACAGGTGGTGGCC |
| | ATTGCCAGCAACATCGGCGGCAAGCAGGCCCTGGAAACCGTGCAGAGACTG |
| | CTGCCCGTGCTGTGCCAGGCCCATGGCCTGACACCTGAACAGGTGGTGGCTA |
| | TCGCCTCTCACGACGGAGGAAAACAGGCTCTGGAAACAGTGCAGCGGCTGCT |
| | GCCTGTGCTGTGTCAGGCTCACGGCTTGACTCCAGAACAGGTGGTGGCTATT |
| | GCTTCCCACGACGGGGGGAAACAGGCCCTGGAAACTGTGCAGCGCNTGCTG |
| | CCAGTGCTGTGCCAGGCTCACGGACTGACCCCCGAACAGGTGGTGGCCATTG |
| | CCAGCAACAACGGCGGCAAGCAGGCCCTGGAAACCGTGCAGAGACTGCTGC |
| | CCGTGCTGTGCCAGGCCCATGGCCTGACACCTGAACAGGTGGTGGCTATCGC |
| | CTCTAACAACGGAGGAAAACAAGCACTCGAGACAGTGCAGCGGCTGCTGCC |
| | TGTGCTGTGTCAGGCTCACGGCTTGACTCCAGAACAGGTGGTGGCTATTGCTT |
| | CCAACGGCGGGGGGAAACAGGCCCTGGAAACTGTGCAGCGCCTGCTGCCAG |
| | TGCTATGCCAGGCTCACGGCCTGACCCCCGAACAGGTGGTGGCCATTGCCAG |
| | CCACGACGGCGGCAAGCAGGCCCTGGAAACCGTGCAGAGACTGCTGCCCGT |
| | GCTGTGCCAGGCCCATGGCCTGACACCTGAACAGGTGGTGGCTATCGCCTCT |
| | CACGACGGAGGAAAACAGGCTCTGGAAACAGTGCAGCGGCTGCTGCCTGTG |
| | CTGTGTCAGGCTCACGGCTTGACTCCACAGCAGGTCGTGGCAATTGCTAGCA |
| | ACGGCGGCGGACGGCCCGCCCTGGAGAGCATTGTGGCCCAGCTGTCTAGACC |
| | GGACCCAGCTCTCGCTGCATTGACTAACGACCATCTCGTCGCTTTGGCTTGCC |
| | TCGGTGGAAGGCCAGCTCTTGACGCTGTAAAGAAAGGTTTGCCACATGCTCC |
| | AGCTCTTATTAAAAGGACTGGTAGCGGTAGCATGAATGAATTAACCTTGCA |
| | CCCTAACGTCTACGCAACAGGAGCCTCCAAGGGTTTGATACACGTATTG |
| | GAAAGGGTGTGGGTAAGGGAGCATACTCCAGGTGATGGAACAATATTTG |
| | TCATTAGCGGATTCGGAAATTATAATGGAGGTGTAAGGTTCTTTGAATC |
| | CTTCAGGGAGCATAAACGCAGGGGAGGTGAATTGGTTGCTGTCTTTGCA |
| | GGATCAACCTCGGCTAGGTTGACTTCTAAACAAGTAGTCCATGAAATGT |
| | TGGACTGTGGTGCAGACGTACATATTGTAAATCGCAAAAGGTTGCTTCA |
| | TGCAAAATGCTATGGTACGTCCAAAATGAGCGGAGACAGCTTGATAGTC |
| | ACAAGCGGTAACTTTACAGGGCCTGGTATGAGCCAAAATGTCGAGATGA |
| | GCGTGCTCTTGGATGCTGACACGACTGCGGAAATGGGATTTAGCTGGGG |
| | TACACTCTTGGAAAATCTGTTGGACCAAAGGTGGAATTATCACAGACCG |
| | ACGCTCGGCGACCCTGCGAACCCTGCATGGTCGTTGCTGTACGACGAAC |
| | AAGCTGCGGACATACGCCTCGACGAGACAGAGGAATGA |
| 117 | MAPSVMASSATTVAPFQGSSPPPACRRPPSELQLRQRQHGGRIRCTGRATMGKPI |
| | PNPLLGLDSTGGVDLRTLGYSQQQQEKIKPKVRSTVAQHHEALVGHGFTHAHIV |
| | ALSQHPAALGTVAVKYQDMIAALPEATHEAIVGVGKRGAGARALEALLTVAGE |
| | LRGPPLQLDTGQLLKIAKRGGVTAVEAVHTWRNALTGAPLNLTPEQVVAIASHD |
| | GGKQALETVQRLLPVLCQAHGLTPEQVVAIASNIGGKQALETVQRLLPVLCQAH |
| | GLTPEQVVAIASNIGGKQALETVQRLLPVLCQAHGLTPEQVVAIASNGGGKQAL |
| | ETVQRLLPVLCQAHGLTPEQVVAIASNGGGKQALETVQRLLPVLCQAHGLTPEQ |
| | VVAIASNNGGKQALETVQRLLPVLCQAHGLTPEQVVAIASNGGGKQALETVQR |
| | LLPVLCQAHGLTPEQVVAIASHDGGKQALETVQRLLPVLCQAHGLTPEQVVAIA |
| | SNIGGKQALETVQRLLPVLCQAHGLTPEQVVAIASNIGGKQALETVQRLLPVLCQ |
| | AHGLTPEQVVAIASHDGGKQALETVQRLLPVLCQAHGLTPEQVVAIASHDGGK |
| | QALETVQRXLPVLCQAHGLTPEQVVAIASNNGGKQALETVQRLLPVLCQAHGL |
| | TPEQVVAIASNNGGKQALETVQRLLPVLCQAHGLTPEQVVAIASNGGGKQALET |
| | VQRLLPVLCQAHGLTPEQVVAIASHDGGKQALETVQRLLPVLCQAHGLTPEQV |
| | VAIASHDGGKQALETVQRLLPVLCQAHGLTPQQVVAIASNGGGRPALESIVAQL |
| | SRPDPALAALTNDHLVALACLGGRPALDAVKKGLPHAPALIKRTGSGSMNELT |
| | LHPNVYATGASKGLIHVLERVWVREHTPGDGTIFVISGFGNYNGGVRFFESF |
| | REHKRRGGELVAVFAGSTSARLTSKQVVHEMLDCGADVHIVNRKRLLHAK |
| | CYGTSKMSGDSLIVTSGNFTGPGMSQNVEMSVLLDADTTAEMGFSWGTLL |
| | ENLLDQRWNYHRPTLGDPANPAWSLLYDEQAADIRLDETEE |
| 118 | ATGGCTCCCAGCGTCATGGCCTCCAGCGCGACAACTGTGGCCCCGTTTCAGG |
| | GTTCGAGCCCGCCACCTGCTTGCCGCCGGCCACCTAGCGAGCTGCAACTCCG |
| | CCAGCGCCAACACGGAGGGAGAATTAGATGCACCGGCCGCGCCACCATGGG |
| | AAAACCGATCCCAAACCCGCTTTTGGGGCTCGACAGCACAGGAGGGGTTGAT |
| | CTCAGGACCCTCGGTTACAGCCAACAGCAACAAGAAAAAATTAAGCCTAAA |
| | GTGAGGTCCACGGTAGCTCAACATCACGAGGCTTTGGTAGGGCATGGCTTCA |
| | CGCACGCTCATATCGTCGCTCTCAGCCAACATCCCGCTGCGCTTGGGACGGT |
| | CGCTGTTAAGTACCAAGACATGATCGCTGCGCTTCCGGAAGCTACGCATGAG |
| | GCGATCGTCGGTGTAGGGAAGCGGGGAGCAGGTGCGAGGGCGCTCGAGGCG |
| | CTCTTGACCGTCGCGGGTGAGCTTAGGGGTCCGCCACTCCAATTGGACACCG |
| | GACAACTTTTGAAAATCGCTAAGAGAGGAGGCGTCACAGCCGTCGAGGCGG |
| | TGCACGCCTGGAGAAATGCCCTGACAGGAGCCCCTCTGAACCTGACCCCCGA |
| | ACAGGTGGTGGCCATTGCCAGCAACATCGGCGGCAAGCAGGCCCTGGAAAC |
| | CGTGCAGAGACTGCTGCCCGTGCTGTGCCAGGCCCATGGCCTGACACCTGAA |
| | CAGGTGGTGGCTATCGCCTCTAACGGCGGAGGAAAACAGGCTCTGGAAACA |
| | GTGCAGCGGCTGCTGCCTGTGCTGTGTCAGGCTCACGGCTTGACTCCAGAAC |
| | AGGTGGTGGCTATTGCTTCCAACAACGGGGGGAAACAGGCCCTGGAAACTGT |
| | GCAGCGCCTGCTGCCAGTGCTGTGCCAGGCTCACGGACTGACCCCCGAACAG |
| | GTGGTGGCCATTGCCAGCAACAACGGCGGCAAGCAGGCCCTGGAAACCGTG |
| | CAGAGACTGCTGCCCGTGCTGTGCCAGGCCCATGGCCTGACACCTGAACAGG |
| | TGGTGGCTATCGCCTCTCACGACGGAGGAAAACAGGCTCTGGAAACAGTGCA |
| | GCGGCTGCTGCCTGTGCTGTGTCAGGCTCACGGCTTGACTCCAGAACAGGTG |
| | GTGGCTATTGCTTCCAACGGCGGGGGGAAACAGGCCCTGGAAACTGTGCAGC |
| | GCCTGCTGCCAGTGCTGTGCCAGGCTCACGGGCTGACCCCCGAACAGGTGGT |
| | GGCCATTGCCAGCCACGACGGCGGCAAGCAGGCCCTGGAAACCGTGCAGAG |
| | ACTGCTGCCCGTGCTGTGCCAGGCCCATGGCCTGACACCTGAACAGGTGGTG |
| | GCTATCGCCTCTAACAACGGAGGAAAACAGGCTCTGGAAACAGTGCAGCGG |
| | CTGCTGCCTGTGCTGTGTCAGGCTCACGGCTTGACTCCAGAACAGGTGGTGG |
| | CTATTGCTTCCAATATTGGGGGGAAACAGGCCCTGGAAACTGTGCAGCGCCT |
| | GCTGCCAGTGCTGTGCCAGGCTCACGGCCTCACTCCCGAACAGGTGGTGGCC |
| | ATTGCCAGCAACATCGGCGGCAAGCAGGCCCTGGAAACCGTGCAGAGACTG |
| | CTGCCCGTGCTGTGCCAGGCCCATGGCCTGACACCTGAACAGGTGGTGGCTA |
| | TCGCCTCTAACGGCGGAGGAAAACAGGCTCTGGAAACAGTGCAGCGGCTGC |
| | TGCCTGTGCTGTGTCAGGCTCACGGCTTGACTCCAGAACAGGTGGTGGCTAT |
| | TGCTTCCCACGACGGGGGGAAACAGGCCCTGGAAACTGTGCAGCGCCTGCTG |
| | CCAGTGCTGTGCCAGGCTCACGGACTGACCCCCGAACAGGTGGTGGCCATTG |
| | CCAGCCACGACGGCGGCAAGCAGGCCCTGGAAACCGTGCAGAGACTGCTGC |
| | CCGTGCTGTGCCAGGCCCATGGCCTGACACCTGAACAGGTGGTGGCTATCGC |
| | CTCTAATATCGGAGGAAAACAAGCACTCGAGACAGTGCAGCGGCTGCTGCCT |
| | GTGCTGTGTCAGGCTCACGGCTTGACTCCAGAACAGGTGGTGGCTATTGCTT |
| | CCAACGGCGGGGGGAAACAGGCCCTGGAAACTGTGCAGCGCCTGCTGCCAG |
| | TGCTGTGCCAGGCTCACGGCCTGACCCCCGAACAGGTGGTGGCCATTGCCAG |
| | CAACATCGGCGGCAAGCAGGCCCTGGAAACCGTGCAGAGACTGCTGCCCGT |
| | GCTGTGCCAGGCCCATGGCCTGACACCTGAACAGGTGGTGGCTATCGCCTCT |
| | AACAACGGAGGAAAACAGGCTCTGGAAACAGTGCAGCGGCTGCTGCCTGTG |
| | CTGTGTCAGGCTCACGGCTTGACTCCACAGCAGGTCGTGGCAATTGCTAGCA |
| | ACGGCGGCGGACGGCCCGCCCTGGAGAGCATTGTGGCCCAGCTGTCTAGACC |
| | TGATCCTGCCCTGGCCGCCCTGACAAATGATCACCTGGTGGCCCTGGCCTGTC |
| | TGGGAGGCAGACCTGCCCTGGATGCCGTGAAAAAAGGACTGCCTCACGCCCC |
| | TGCCCTGATCAAGAGAACAGGATCCGGATCCATGAACGAGTTAACGCTGCA |
| | TCCGAATGTGTATGCGACCGGCGCGAGTAAAGGGCTGATACATGTGCTG |
| | GAGCGCGTATGGGTTCGAGAACACACGCCTGGGGACGGCACGATCTTC |
| | GTGATATCTGGCTTTGGCAACTACAACGGTGGCGTTCGCTTCTTCGAGT |
| | CGTTTCGTGAACACAAGAGACGTGGCGGGGAGCTGGTAGCGGTGTTCG |
| | CTGGCAGCACAAGCGCGCGACTGACAAGCAAACAGGTTGTTCACGAGAT |
| | GCTTGATTGCGGGGCGGATGTTCACATAGTGAACAGAAAGCGACTCCTT |
| | CACGCTAAGTGTTACGGCACAAGCAAGATGTCAGGTGATTCCCTAATTG |
| | TAACCTCAGGCAATTTCACAGGTCCGGGAATGTCGCAGAACGTGGAAAT |
| | GTCTGTATTGCTAGACGCAGATACAACCGCCGAGATGGGGTTCTCGTGG |
| | GGCACGTTGCTTGAGAACTTGCTTGATCAGCGATGGAACTACCATCGCC |
| | CCACCTTAGGCGATCCAGCAAATCCAGCCTGGAGCCTGCTTTATGATGA |
| | GCAGGCAGCCGATATTAGGTTGGATGAAACTGAAGAATGA |
| 119 | MAPSVMASSATTVAPFQGSSPPPACRRPPSELQLRQRQHGGRIRCTGRATMGKPI |
| | PNPLLGLDSTGGVDLRTLGYSQQQQEKIKPKVRSTVAQHHEALVGHGFTHAHIV |
| | ALSQHPAALGTVAVKYQDMIAALPEATHEAIVGVGKRGAGARALEALLTVAGE |
| | LRGPPLQLDTGQLLKIAKRGGVTAVEAVHAWRNALTGAPLNLTPEQVVAIASNI |
| | GGKQALETVQRLLPVLCQAHGLTPEQVVAIASNGGGKQALETVQRLLPVLCQA |
| | HGLTPEQVVAIASNNGGKQALETVQRLLPVLCQAHGLTPEQVVAIASNNGGKQ |
| | ALETVQRLLPVLCQAHGLTPEQVVAIASHDGGKQALETVQRLLPVLCQAHGLTP |
| | EQVVAIASNGGGKQALETVQRLLPVLCQAHGLTPEQVVAIASHDGGKQALETV |
| | QRLLPVLCQAHGLTPEQVVAIASNNGGKQALETVQRLLPVLCQAHGLTPEQVV |
| | AIASNIGGKQALETVQRLLPVLCQAHGLTPEQVVAIASNIGGKQALETVQRLLPV |
| | LCQAHGLTPEQVVAIASNGGGKQALETVQRLLPVLCQAHGLTPEQVVAIASHDG |
| | GKQALETVQRLLPVLCQAHGLTPEQVVAIASHDGGKQALETVQRLLPVLCQAH |
| | GLTPEQVVAIASNIGGKQALETVQRLLPVLCQAHGLTPEQVVAIASNGGGKQAL |
| | ETVQRLLPVLCQAHGLTPEQVVAIASNIGGKQALETVQRLLPVLCQAHGLTPEQ |
| | VVAIASNNGGKQALETVQRLLPVLCQAHGLTPQQVVAIASNGGGRPALESIVAQ |
| | LSRPDPALAALTNDHLVALACLGGRPALDAVKKGLPHAPALIKRTGSGSMNEL |
| | TLHPNVYATGASKGLIHVLERVWVREHTPGDGTIFVISGFGNYNGGVRFFES |
| | FREHKRRGGELVAVFAGSTSARLTSKQVVHEMLDCGADVHIVNRKRLLHA |
| | KCYGTSKMSGDSLIVTSGNFTGPGMSQNVEMSVLLDADTTAEMGFSWGTL |
| | LENLLDQRWNYHRPTLGDPANPAWSLLYDEQAADIRLDETEE |
| 120 | AGACATATATATCTATCTAAATTTATTGATATCAATATGAATGTGGAAAATG |
| | CTAAAATAACTTACATTGTGAAATGAAGGAAGTAATTTTCTTCACAAATTTTG |
| | GCACTTGCACTTACGGCTTGTGAAGCCCGCTCGATCTGAAGATTTTTCAAACG |
| | ATGCCATTCACCACGTGCCACTTTATATCAAACCTTATTTGTATCCAAGATTT |
| | CTGGTACTGGGCATTAGCGATGTCAAACAAACTCGACGTAAAGTTGAGGGAC |
| | AGAGTGTGAACTTATTTTATTCCCAGCAGAGAACGCCTCCAAGGTTTATGGG |
| | CTTTCTGGTCCATCGCAACGACACGGACTTTCTACAGGGCTGAGATTACGGG |
| | CCTAAATGGGCTCCCCCGTCTTGTGCTCCGCGCTGTTTCAAGTTGGGCCAAAA |
| | TGGAACTGGGCCGAATAGTCCGGTGCGGCTAACGGCATCCACAGCCAAGCC |
| | AACGGTGCCGTTCTCCCGCTCTCGCATTTCAAAAATCGAAAATAAAATCGGC |
| | AGGGCCGCCGACGACGCAACCACCCGTTGGGATCCAATCTACTCCTACGCAC |
| | CGAACAACTCATCGCACCCACCGATATGTGGGACCCAGCTGTAACGTGGGCC |
| | CACCGGCAAGCGGGACGTCTGCATCGGTCGCACCTACAACGAGGTGGCCAG |
| | GTCACCTACTGCTTTTTCTTGGAGCGACGCGTGCACTCGCTAACCGCACACGA |
| | AGGAAGCCTCTGGACCCACCTGTCAGTGAAACTAACGAGAGGCGGGTATGG |
| | ACTTCAACCGAGTGCAAACGAAATGGCCTCCTTGCGGTGGATATTATCAGAA |
| | AGCTCCCTAACTTATTAAATATTCTCGATATGATCCAACCACCCGACCTTTGC |
| | AGGCACGATGTGAGATGCGAGTGAATTTTGAAGACGTCAAAGGGGGTAAAA |
| | CGAAAAACTCCCCCATCCCTTCGCTTCCGCTCCTGAGAGGTCCAGGCTGTCTG |
| | GCCCTATAAAACGGGCGGAGTGCGGGGATCGCAAACCCTAAGCCGAGACAA |
| | CGCAGAGAAAGGCGTCTTCGTACTCGCCTCTCTCCGCGCCTCCGCGCTTTTCC |
| | TCCTCTCCCCTCTCTCCCTTCTCCGCCGCCGTCGCAGCATCAACCCAATCCGC |
| | CGCCAAGCTTCATCCAGGTACGGATCCGCGTCCCATCTCCCTCACCCCCCGTG |
| | TTCTTCGTGCCTGCTTCTGGGTCAGATCTGGGTGGATTCGCGGTTGTTGGATG |
| | TGGGGGGCTGTGTTTATTTGTCGGTGGATCTGGTTGTCTGGATCTGCGTTTTC |
| | TCTGTCGTAGTTAGCGGATCTGATGAAATGTTTAGTGTTCGTGTATACTGGTA |
| | TGGTGGATCTGGTCCTAGGATGCGTGGAATGGATATATGTAGGCGAATTGGA |
| | GGATTTATTTTGTGAATTTTGCTGAAATGATAGTTCTAAACACTGGATCTGAC |
| | CTCGGGATGCTGTTAAATGTGGAAATCATGGTCGATGCTGTCATGAACATGG |
| | TGTTCTTATGGTAGATCTGAGCAATGTATGTTTCAAAATTGTTTGTCACATGG |
| | AAATGCTATGGTTCTAGATGCAATAGAATGATACATGCCGAGATCCCCTCTA |
| | GTTGATATGATAGATCATGATGTTTTACAGCTATGTCATATGAATATGTTCAT |
| | TTGTTACCGATGTATTTGGATCTACTTAACATTTCCAAAGCACGCCGCGTTCT |
| | AATTCTAGATCTGGTAGTCATGTTTGTACACGTCATCCACCTAATACAAATAC |
| | ATATGTCTAGTGTTTGGTGACACTGCCCGTCAGATCTGTTTTTTCCAGATCTG |
| | TGGAACAAATACTCCATGCATGTATGGTAGTTTTGAAACGATCTTGTATCTTC |
| | CATTGTTGTAGTAACAACTAAATAAAGTACAATTGTTCAATTATTGGGAATC |
| | GTATTTTCTGTAGTGCCGATGTACAGCATATTCATAGATGTCTATTTAGGAAC |
| | TCAAATTTTAAATTGAGGACTAGTTATTTATTATGGGTCAGTCTTTTGAATTG |
| | TGTTATCTTGCTGTACTGAAATAATAATGTACCACTAAGGCGCTAACATGTAT |
| | TTTGTCCTTTCAGGCTGACTCGACGGATCCCGGGGGGCAAGAGCTCCTCCCC |
| | CCTCCCCCTCCGCCGCCGCCGGTAACCACCCCGCCCCTCTCCTCTTTCTTTCTC |
| | CGTTTTTTTTTTCGTCTCGGTCTCGATCTTTGGCCTTGGTAGTTTGGGTGGGCG |
| | AGAGCGGCTTCGTCGCCCAGATCGGTGCGCGGGAGGGGCGGGATCTCGCGG |
| | CTGGCGTCTCCGGGCGTGAGTCGGCCCGGATCCTCGCGGGGAATGGGGCTCT |
| | CGGATGTAGATCTTCTTTCTTTCTTCTTTTTGTGGTAGAATTTGAATCCCTCAG |
| | CATTGTTCATCGGTAGTTTTTCTTTTCATGATTTGTGACAAATGCAGCCTCGT |
| | GCGGAGCTTTTTTGTAGGTAGAAGATGGCCCCCTCCGTGATGGCGTCGTC |
| | GGCCACCACCGTCGCTCCCTTCCAGGGCTCAAGTCCACCGCCGGCTTGC |
| | CGTCGCCCGCCGTCCGAACTCCAGCTTCGGCAACGTCAGCATGGCGGCA |
| | GGATCAGATGCACCGGCCGCGCCACCATGGGAAAACCTATTCCTAATCC |
| | TCTGCTGGGCCTGGATTCTACCGGAGGCGTGGACCTGAGAACACTGGGA |
| | TATTCTCAGCAGCAGCAGGAGAAGATCAAGCCCAAGGTGAGATCTACAG |
| | TGGCCCAGCACCACGAAGCCCTGGTGGGACACGGATTTACACACGCCCA |
| | CATTGTGGCCCTGTCTCAGCACCCTGCCGCCCTGGGAACAGTGGCCGTG |
| | AAATATCAGGATATGATTGCCGCCCTGCCTGAGGCCACACACGAAGCCA |
| | TTGTGGGAGTGGGAAAACGAGGCGCTGGAGCCAGAGCCCTGGAAGCCC |
| | TGCTGACAGTGGCCGGAGAACTGAGAGGACCTCCTCTGCAGCTGGATAC |
| | AGGACAGCTGCTGAAGATTGCCAAAAGGGGCGGAGTGACCGCGGTGGA |
| | AGCCGTGCACACCTGGAGAAATGCCCTGACAGGAGCCCCTCTGAACCTG |
| | ACCCCCGAACAGGTGGTGGCCATTGCCAGCCACGACGGCGGCAAGCAG |
| | GCCCTGGAAACCGTGCAGAGACTGCTGCCCGTGCTGTGCCAGGCCCATG |
| | GCCTGACACCTGAACAGGTGGTGGCTATCGCCTCTAATATCGGAGGAAA |
| | ACAGGCTCTGGAAACAGTGCAGCGGCTGCTGCCTGTGCTGTGTCAGGCT |
| | CACGGCTTGACTCCAGAACAGGTGGTGGCTATTGCTTCCAATATTGGGG |
| | GGAAACAGGCCCTGGAAACTGTGCAGCGCCTGCTGCCAGTGCTGTGCCA |
| | GGCTCACGGACTGACCCCCGAACAGGTGGTGGCCATTGCCAGCAACGG |
| | CGGCGGCAAGCAGGCCCTGGAAACCGTGCAGAGACTGCTGCCCGTGCT |
| | GTGCCAGGCCCATGGCCTGACACCTGAACAGGTGGTGGCTATCGCCTCT |
| | AACGGCGGAGGAAAACAGGCTCTGGAAACAGTGCAGCGGCTGCTGCCT |
| | GTGCTGTGTCAGGCTCACGGCTTGACTCCAGAACAGGTGGTGGCTATTG |
| | CTTCCAACAACGGGGGGAAACAGGCCCTGGAAACTGTGCAGCGCCTGCT |
| | GCCAGTGCTGTGCCAGGCTCACGGGCTGACCCCCGAACAGGTGGTGGC |
| | CATTGCCAGCAACGGCGGCGGCAAGCAGGCCCTGGAAACCGTGCAGAG |
| | ACTGCTGCCCGTGCTGTGCCAGGCCCATGGCCTGACACCTGAACAGGTG |
| | GTGGCTATCGCCTCTCACGACGGAGGAAAACAGGCTCTGGAAACAGTGC |
| | AGCGGCTGCTGCCTGTGCTGTGTCAGGCTCACGGCTTGACTCCAGAACA |
| | GGTGGTGGCTATTGCTTCCAACATCGGGGGGAAACAGGCCCTGGAAACT |
| | GTGCAGCGCCTGCTGCCAGTGCTGTGCCAGGCTCACGGCCTCACTCCCG |
| | AACAGGTGGTGGCCATTGCCAGCAACATCGGCGGCAAGCAGGCCCTGG |
| | AAACCGTGCAGAGACTGCTGCCCGTGCTGTGCCAGGCCCATGGCCTGAC |
| | ACCTGAACAGGTGGTGGCTATCGCCTCTCACGACGGAGGAAAACAGGCT |
| | CTGGAAACAGTGCAGCGGCTGCTGCCTGTGCTGTGTCAGGCTCACGGCT |
| | TGACTCCAGAACAGGTGGTGGCTATTGCTTCCCACGACGGGGGGAAACA |
| | GGCCCTGGAAACTGTGCAGCGCNTGCTGCCAGTGCTGTGCCAGGCTCAC |
| | GGACTGACCCCCGAACAGGTGGTGGCCATTGCCAGCAACAACGGCGGC |
| | AAGCAGGCCCTGGAAACCGTGCAGAGACTGCTGCCCGTGCTGTGCCAG |
| | GCCCATGGCCTGACACCTGAACAGGTGGTGGCTATCGCCTCTAACAACG |
| | GAGGAAAACAAGCACTCGAGACAGTGCAGCGGCTGCTGCCTGTGCTGT |
| | GTCAGGCTCACGGCTTGACTCCAGAACAGGTGGTGGCTATTGCTTCCAA |
| | CGGCGGGGGGAAACAGGCCCTGGAAACTGTGCAGCGCCTGCTGCCAGT |
| | GCTATGCCAGGCTCACGGCCTGACCCCCGAACAGGTGGTGGCCATTGCC |
| | AGCCACGACGGCGGCAAGCAGGCCCTGGAAACCGTGCAGAGACTGCTG |
| | CCCGTGCTGTGCCAGGCCCATGGCCTGACACCTGAACAGGTGGTGGCTA |
| | TCGCCTCTCACGACGGAGGAAAACAGGCTCTGGAAACAGTGCAGCGGCT |
| | GCTGCCTGTGCTGTGTCAGGCTCACGGCTTGACTCCACAGCAGGTCGTG |
| | GCAATTGCTAGCAACGGCGGCGGACGGCCCGCCCTGGAGAGCATTGTG |
| | GCCCAGCTGTCTAGACCGGACCCAGCTCTCGCTGCATTGACTAACGACC |
| | ATCTCGTCGCTTTGGCTTGCCTCGGTGGAAGGCCAGCTCTTGACGCTGT |
| | AAAGAAAGGTTTGCCACATGCTCCAGCTCTTATTAAAAGGACTGGTAGC |
| | GGTAGCATGAATGAATTAACCTTGCACCCTAACGTCTACGCAACAGGAG |
| | CCTCCAAGGGTTTGATACACGTATTGGAAAGGGTGTGGGTAAGGGAGCA |
| | TACTCCAGGTGATGGAACAATATTTGTCATTAGCGGATTCGGAAATTATA |
| | ATGGAGGTGTAAGGTTCTTTGAATCCTTCAGGGAGCATAAACGCAGGGG |
| | AGGTGAATTGGTTGCTGTCTTTGCAGGATCAACCTCGGCTAGGTTGACT |
| | TCTAAACAAGTAGTCCATGAAATGTTGGACTGTGGTGCAGACGTACATA |
| | TTGTAAATCGCAAAAGGTTGCTTCATGCAAAATGCTATGGTACGTCCAA |
| | AATGAGCGGAGACAGCTTGATAGTCACAAGCGGTAACTTTACAGGGCCT |
| | GGTATGAGCCAAAATGTCGAGATGAGCGTGCTCTTGGATGCTGACACGA |
| | CTGCGGAAATGGGATTTAGCTGGGGTACACTCTTGGAAAATCTGTTGGA |
| | CCAAAGGTGGAATTATCACAGACCGACGCTCGGCGACCCTGCGAACCCT |
| | GCATGGTCGTTGCTGTACGACGAACAAGCTGCGGACATACGCCTCGACG |
| | AGACAGAGGAATGAGAATTCATATGAAGATGAAGATGAAATATTTGGTGTG |
| | TCAAATAAAAAGCTTGTGTGCTTAAGTTTGTGTTTTTTTCTTGGCTTGTTGTGT |
| | TATGAATTTGTGGCTTTTTCTAATATTAAATGAATGTAAGATCTCATTATAAT |
| | GAATAAACAAATGTTTCTATAATCCATTGTGAATGTTTTGTTGGATCTCTTCT |
| | GCAGCATATAACTACTGTATGTGCTATGGTATGGACTATGGAATATGATTAA |
| | AGATAAG |
| 121 | CTGCAGTGCAGCGTGACCCGGTCGTGCCCCTCTCTAGAGATAATGAGCATTG |
| | CATGTCTAAGTTATAAAAAATTACCACATATTTTTTTTGTCACACTTGTTTGA |
| | AGTGCAGTTTATCTATCTTTATACATATATTTAAACTTTACTCTACGAATAAT |
| | ATAATCTATAGTACTACAATAATATCAGTGTTTTAGAGAATCATATAAATGA |
| | ACAGTTAGACATGGTCTAAAGGACAATTGAGTATTTTGACAACAGGACTCTA |
| | CAGTTTTATCTTTTTAGTGTGCATGTGTTCTCCTTTTTTTTTGCAAATAGCTTC |
| | ACCTATATAATACTTCATCCATTTTATTAGTACATCCATTTAGGGTTTAGGGT |
| | TAATGGTTTTTATAGACTAATTTTTTTAGTACATCTATTTTATTCTATTTTAGC |
| | CTCTAAATTAAGAAAACTAAAACTCTATTTTAGTTTTTTTATTTAATAATTTA |
| | GATATAAAATAGAATAAAATAAAGTGACTAAAAATTAAACAAATACCCTTTA |
| | AGAAATTAAAAAAACTAAGGAAACATTTTTCTTGTTTCGAGTAGATAATGCC |
| | AGCCTGTTAAACGCCGTCGACGAGTCTAACGGACACCAACCAGCGAACCAG |
| | CAGCGTCGCGTCGGGCCAAGCGAAGCAGACGGCACGGCATCTCTGTCGCTGC |
| | CTCTGGACCCCTCTCGAGAGTTCCGCTCCACCGTTGGACTTGCTCCGCTGTCG |
| | GCATCCAGAAATTGCGTGGCGGAGCGGCAGACGTGAGCCGGCACGGCAGGC |
| | GGCCTCCTCCTCCTCTCACGGCACCGGCAGCTACGGGGGATTCCTTTCCCACC |
| | GCTCCTTCGCTTTCCCTTCCTCGCCCGCCGTAATAAATAGACACCCCCTCCAC |
| | ACCCTCTTTCCCCAACCTCGTGTTGTTCGGAGCGCACACACACACAACCAGA |
| | TCTCCCCCAAATCCACCCGTCGGCACCTCCGCTTCAAGGTACGCCGCTCGTCC |
| | TCCCCCCCCCCCCCTCTCTACCTTCTCTAGATCGGCGTTCCGGTCCATGGTTA |
| | GGGCCCGGTAGTTCTACTTCTGTTCATGTTTGTGTTAGATCCGTGTTTGTGTTA |
| | GATCCGTGCTGCTAGCGTTCGTACACGGATGCGACCTGTACGTCAGACACGT |
| | TCTGATTGCTAACTTGCCAGTGTTTCTCTTTGGGGAATCCTGGGATGGCTCTA |
| | GCCGTTCCGCAGACGGGATCGATTTCATGATTTTTTTTGTTTCGTTGCATAGG |
| | GTTTGGTTTGCCCTTTTCCTTTATTTCAATATATGCCGTGCACTTGTTTGTCGG |
| | GTCATCTTTTCATGCTTTTTTTTGTCTTGGTTGTGATGATGTGGTCTGGTTGGG |
| | CGGTCGTTCTAGATCGGAGTAGAATTAATTCTGTTTCAAACTACCTGGTGGAT |
| | TTATTAATTTTGGATCTGTATGTGTGTGCCATACATATTCATAGTTACGAATT |
| | GAAGATGATGGATGGAAATATCGATCTAGGATAGGTATACATGTTGATGCGG |
| | GTTTTACTGATGCATATACAGAGATGCTTTTTGTTCGCTTGGTTGTGATGATG |
| | TGGTGTGGTTGGGCGGTCGTTCATTCGTTCTAGATCGGAGTAGAATACTGTTT |
| | CAAACTACCTGGTGTATTTATTAATTTTGGAACTGTATGTGTGTGTCATACAT |
| | CTTCATAGTTACGAGTTTAAGATGGATGGAAATATCGATCTAGGATAGGTAT |
| | ACATGTTGATGTGGGTTTTACTGATGCATATACATGATGGCATATGCAGCATC |
| | TATTCATATGCTCTAACCTTGAGTACCTATCTATTATAATAAACAAGTATGTT |
| | TTATAATTATTTTGATCTTGATATACTTGGATGATGGCATATGCAGCAGCTAT |
| | ATGTGGATTTTTTTAGCCCTGCCTTCATACGCTATTTATTTGCTTGGTACTGTT |
| | TCTTTTGTCGATGCTCACCCTGTTGTTTGGTGTTACTTCTGCAGGTCGACTCTA |
| | GAGGATCCATGGCTCCCAGCGTCATGGCCTCCAGCGCGACAACTGTGGC |
| | CCCGTTTCAGGGTTCGAGCCCGCCACCTGCTTGCCGCCGGCCACCTAGC |
| | GAGCTGCAACTCCGCCAGCGCCAACACGGAGGGAGAATTAGATGCACC |
| | GGCCGCGCCACCATGGGAAAACCGATCCCAAACCCGCTTTTGGGGCTCG |
| | ACAGCACAGGAGGGGTTGATCTCAGGACCCTCGGTTACAGCCAACAGCA |
| | ACAAGAAAAAATTAAGCCTAAAGTGAGGTCCACGGTAGCTCAACATCAC |
| | GAGGCTTTGGTAGGGCATGGCTTCACGCACGCTCATATCGTCGCTCTCA |
| | GCCAACATCCCGCTGCGCTTGGGACGGTCGCTGTTAAGTACCAAGACAT |
| | GATCGCTGCGCTTCCGGAAGCTACGCATGAGGCGATCGTCGGTGTAGG |
| | GAAGCGGGGAGCAGGTGCGAGGGCGCTCGAGGCGCTCTTGACCGTCGC |
| | GGGTGAGCTTAGGGGTCCGCCACTCCAATTGGACACCGGACAACTTTTG |
| | AAAATCGCTAAGAGAGGAGGCGTCACAGCCGTCGAGGCGGTGCACGCC |
| | TGGAGAAATGCCCTGACAGGAGCCCCTCTGAACCTGACCCCCGAACAGG |
| | TGGTGGCCATTGCCAGCAACATCGGCGGCAAGCAGGCCCTGGAAACCG |
| | TGCAGAGACTGCTGCCCGTGCTGTGCCAGGCCCATGGCCTGACACCTGA |
| | ACAGGTGGTGGCTATCGCCTCTAACGGCGGAGGAAAACAGGCTCTGGA |
| | AACAGTGCAGCGGCTGCTGCCTGTGCTGTGTCAGGCTCACGGCTTGACT |
| | CCAGAACAGGTGGTGGCTATTGCTTCCAACAACGGGGGGAAACAGGCC |
| | CTGGAAACTGTGCAGCGCCTGCTGCCAGTGCTGTGCCAGGCTCACGGAC |
| | TGACCCCCGAACAGGTGGTGGCCATTGCCAGCAACAACGGCGGCAAGC |
| | AGGCCCTGGAAACCGTGCAGAGACTGCTGCCCGTGCTGTGCCAGGCCC |
| | ATGGCCTGACACCTGAACAGGTGGTGGCTATCGCCTCTCACGACGGAGG |
| | AAAACAGGCTCTGGAAACAGTGCAGCGGCTGCTGCCTGTGCTGTGTCAG |
| | GCTCACGGCTTGACTCCAGAACAGGTGGTGGCTATTGCTTCCAACGGCG |
| | GGGGGAAACAGGCCCTGGAAACTGTGCAGCGCCTGCTGCCAGTGCTGT |
| | GCCAGGCTCACGGGCTGACCCCCGAACAGGTGGTGGCCATTGCCAGCC |
| | ACGACGGCGGCAAGCAGGCCCTGGAAACCGTGCAGAGACTGCTGCCCG |
| | TGCTGTGCCAGGCCCATGGCCTGACACCTGAACAGGTGGTGGCTATCGC |
| | CTCTAACAACGGAGGAAAACAGGCTCTGGAAACAGTGCAGCGGCTGCTG |
| | CCTGTGCTGTGTCAGGCTCACGGCTTGACTCCAGAACAGGTGGTGGCTA |
| | TTGCTTCCAATATTGGGGGGAAACAGGCCCTGGAAACTGTGCAGCGCCT |
| | GCTGCCAGTGCTGTGCCAGGCTCACGGCCTCACTCCCGAACAGGTGGTG |
| | GCCATTGCCAGCAACATCGGCGGCAAGCAGGCCCTGGAAACCGTGCAG |
| | AGACTGCTGCCCGTGCTGTGCCAGGCCCATGGCCTGACACCTGAACAGG |
| | TGGTGGCTATCGCCTCTAACGGCGGAGGAAAACAGGCTCTGGAAACAGT |
| | GCAGCGGCTGCTGCCTGTGCTGTGTCAGGCTCACGGCTTGACTCCAGAA |
| | CAGGTGGTGGCTATTGCTTCCCACGACGGGGGGAAACAGGCCCTGGAA |
| | ACTGTGCAGCGCCTGCTGCCAGTGCTGTGCCAGGCTCACGGACTGACCC |
| | CCGAACAGGTGGTGGCCATTGCCAGCCACGACGGCGGCAAGCAGGCCC |
| | TGGAAACCGTGCAGAGACTGCTGCCCGTGCTGTGCCAGGCCCATGGCCT |
| | GACACCTGAACAGGTGGTGGCTATCGCCTCTAATATCGGAGGAAAACAA |
| | GCACTCGAGACAGTGCAGCGGCTGCTGCCTGTGCTGTGTCAGGCTCACG |
| | GCTTGACTCCAGAACAGGTGGTGGCTATTGCTTCCAACGGCGGGGGGAA |
| | ACAGGCCCTGGAAACTGTGCAGCGCCTGCTGCCAGTGCTGTGCCAGGCT |
| | CACGGCCTGACCCCCGAACAGGTGGTGGCCATTGCCAGCAACATCGGC |
| | GGCAAGCAGGCCCTGGAAACCGTGCAGAGACTGCTGCCCGTGCTGTGC |
| | CAGGCCCATGGCCTGACACCTGAACAGGTGGTGGCTATCGCCTCTAACA |
| | ACGGAGGAAAACAGGCTCTGGAAACAGTGCAGCGGCTGCTGCCTGTGC |
| | TGTGTCAGGCTCACGGCTTGACTCCACAGCAGGTCGTGGCAATTGCTAG |
| | CAACGGCGGCGGACGGCCCGCCCTGGAGAGCATTGTGGCCCAGCTGTC |
| | TAGACCTGATCCTGCCCTGGCCGCCCTGACAAATGATCACCTGGTGGCC |
| | CTGGCCTGTCTGGGAGGCAGACCTGCCCTGGATGCCGTGAAAAAAGGA |
| | CTGCCTCACGCCCCTGCCCTGATCAAGAGAACAGGATCCGGATCCATGA |
| | ACGAGTTAACGCTGCATCCGAATGTGTATGCGACCGGCGCGAGTAAAGG |
| | GCTGATACATGTGCTGGAGCGCGTATGGGTTCGAGAACACACGCCTGGG |
| | GACGGCACGATCTTCGTGATATCTGGCTTTGGCAACTACAACGGTGGCG |
| | TTCGCTTCTTCGAGTCGTTTCGTGAACACAAGAGACGTGGCGGGGAGCT |
| | GGTAGCGGTGTTCGCTGGCAGCACAAGCGCGCGACTGACAAGCAAACA |
| | GGTTGTTCACGAGATGCTTGATTGCGGGGCGGATGTTCACATAGTGAAC |
| | AGAAAGCGACTCCTTCACGCTAAGTGTTACGGCACAAGCAAGATGTCAG |
| | GTGATTCCCTAATTGTAACCTCAGGCAATTTCACAGGTCCGGGAATGTC |
| | GCAGAACGTGGAAATGTCTGTATTGCTAGACGCAGATACAACCGCCGAG |
| | ATGGGGTTCTCGTGGGGCACGTTGCTTGAGAACTTGCTTGATCAGCGAT |
| | GGAACTACCATCGCCCCACCTTAGGCGATCCAGCAAATCCAGCCTGGAG |
| | CCTGCTTTATGATGAGCAGGCAGCCGATATTAGGTTGGATGAAACTGAA |
| | GAATGAGAATTCGGTACCGAGTTTGAATCAAATCTTCACTTGTTTAATGAGA |
| | TATGCGAGACGCCTATGATCGCATGATATTTGCTTTCAATTCTGTTGTGCACG |
| | TTGTAAAAAACCTGAGCATGTGTAGCTCAGATCCTTACCGCCGGTTTCGGTTC |
| | ATTCTAATGAATATATCACCCGTTACTATCGTATTTTTATGAATAATATTCTC |
| | CGTTCAATTTACTGATTGTACCCTACTACTTATATGTACAATATTAAAATGAA |
| | AACAATATATTGTGCTGAATAGGTTTATAGCGACATCTATGATAGAGCGCCA |
| | CAATAACAAACAATTGCGTTTTATTATTACAAATCCAATTTTAAAAAAAGCG |
| | GCAGAACCGGTCAAACCTAAAAGACTGATTACATAAATCTTATTCAAATTTC |
| | AAAAGTGCCCCAGGGGCTAGTATCTACGACACACCGAGCGGCGAATTCAGT |
| | ACATTAAAAACGTCCGCAATGTGTTATTAAGTTGTCTAAGCGTCAATTTGTTT |
| | ACACCACAATATATCCTGCCACCAGCCAGCCAACAGCTCCCCGACCGGCAGC |
| | TCGGCACAAAATCACCACTCGATACAGGCAGCCCATCAGTCCGGGACGGCGT |
| | CAGCGGGA |
| 122 | CCTCCGGGCTTTCTTTCCACACTATTATGGATAGTCAAATAATGGGAAAAATT |
| | GGATTCAATTGTCAACCGGTCCTATCGAAAATAGGATTGACTATGGATTCGA |
| | GCCATAGCACATGGTTTCATAAAATCTGTACGATTTT |
| 123 | CTCCGGGCTTTCTTTCCACAC |
| 124 | AATCGTACAGATTTTATGAAACCATGTGCT |
| 125 | GAAAAGGTCAAGTCTAAGCGACATATGGCACGAAAAGGAAATCCAATTTCG |
| | GTAATCCGAAGCCCACCTTACCTTAATCCATTTTCTTGAGGATCTTGCATTTG |
| | TTCGTTCTCTGCCGACCTATTGTTTCCATCACTCCTGTTGCTTCTTCCTTTCTCT |
| | ATCCAACCCGAGCTCTATCGCCCATGGCTGAACTCTTCTCTGCAGCCTCATCG |
| | CCAACAAAAGGTCCACATCCTTCAATCGGCATGTACTATGGAATGGCTTTAA |
| | TGTTTCTATATCCGGAAGTAGGGAATCTCGTGCTTGCATAATATGCCATTCA |
| | AGTGCTGGATGAGCTGACTAAAGGTGAGGCAATCATCGCTACTGGTGTT |
| | GGGCAACATCAAATGTGGGCGGCACAATATTATACCTATAAGCGACCAC |
| | GACAATGGCTGTCTTCGGCTGGTCTGGGCGCAATGGGATTTGGGCTGCC |
| | TGCTGCAGCTGGTGCTTCTGTGGCTAACCCAGGTGTCACAGTTGTTGAT |
| | ATTGATGGGGATGGTAGCTTCCTCATGAACATTCAAGAGCTGGCATTGA |
| | TCCGTATTGAGAACCTCCCTGTGAAGGTGATGGTGTTGAACAACCAACA |
| | TTTGGGTATGGTGGTGCAATTGGAGGATAGGTTTTATAAGGCGAATAGG |
| | GCGCATACATATTTGGGCAACCCAGAATGTGAGAGCGAGATATATCCAG |
| | ATTTTGTGACTAT |
| 126 | MTRDRMRQSIKGRALCRAFSSRSAGGVIFKVVLKSLQERKKRISSEISSTIYFYML |
| | CIPYLIFFLKISILVFLVFYRIVLPIVQMFSLLTCCSFLILILLFCLILTCLILFRLKKKE |
| | DFASPPESIISIFSSLFAILMAVLASGPSVAYAMENPGPSEAGPEGSAAPPSSSFQES |
| | TGEMDALMASTTPSAPRTPSGGEPSVNQPLPGEQAMPPALPVMQEAANRAPPYA |
| | PYPYPVDEIIGGDSVQSIQRRLLGTNWNPSAHDIKMSRIQAEDLFELKVEIIRKMA |
| | GLHPSGDWMGWGARALDNPRTATGEEDLARLHQMLDDLQSRNEQSATFWRLV |
| | ERVRLRADEDQNSAS |
| 127 | MTRDRMRQSIKGRALCRAFSSRSAGGVIFKVVLKSLQERKKRISSEISSTIYFYML |
| | CIPYLIFFLKISILVFLVFYRIVLPIVQMFSLLTCCSFLILILLFCLILTCLILFRLKKKE |
| | DFASPPESIISIFSSIFAILMAVLASGPSVAYAMENPGPSEAGPEGSAAPPSSSFQES |
| | TGEMDALMASTTPSAPRTPSGGEPSVNQPLPGEQAMPPALPVMQEAANRSPPYA |
| | PYPYPVDEIIGGDSVQSIQRRLLGTNWNPSAHDMQMSRIQAEDLFELKVEIIRKM |
| | AGLHPSGDWMGWGARALDNPRTATGEEDLARLHQMLDDLQSRNEQSATFWRL |
| | VERVRLRADEDQNSAS |
| 128 | MTRDRMRQSIKGRALCRAFSSRSAGGVIFKVVLKSLQERKKRISSEISSTIYFYML |
| | CIPYLIFFLKISILVFLVFYRIVLPIVQMFSLLTCCSFLILILLFCLILTCLILFRLKKKE |
| | DFASPPESIISIFSSIFAILMAVLASGPSVAYAMENPGPSEAGPEGSAAPPSSSFQES |
| | TGEMDALMASTTPSAPRTPSGGEPSVNQPLPGEQAMPPALPVMQEAANRSPPYA |
| | PYPYPVDEIIGGDSVQSIQRRLLGTNWNPSAHDMQMSRIQAEDLFELKVEIIRKM |
| | AGLHPSGDWMGWGARALDNPRTATGEEDLARLHQMLDDLQSRNEQSATFWRL |
| | VERVRLRADEDQNSAS |
| 129 | MALRRAVASFLPKLAGAAETLPAASHAASSFSQLICTPLDVVERQQQPSGFRSFA |
| | SDAV |

What is claimed is:

1. A method for replacing an endogenous target polynucleotide present in a genome of a mitochondrion or a plastid of a cell with an internal polynucleotide, the method comprising:
(a) introducing into the mitochondrion or the plastid of the cell a replacement nucleic acid comprising the internal polynucleotide,
wherein the internal polynucleotide comprises a sequence heterologous to the genome of the mitochondrion or the plastid;
(b) selecting an altered cell comprising at least one altered mitochondrion genome or at least one altered plastid genome, wherein the at least one altered mitochondrion genome or the at least one altered plastid genome comprises the internal polynucleotide;
(c) introducing into the altered cell a polynucleotide encoding a site-directed nuclease (SDN), wherein the SDN binds to a target sequence present in the endogenous target polynucleotide; and
(d) selecting a further altered cell, wherein at least about 50% of mitochondrial genomes of a plurality of mitochondrial genomes or at least about 50% of plastid genomes of a plurality of plastid genomes in the further altered cell comprise the internal polynucleotide.

2. The method of claim 1, wherein the replacement nucleic acid further comprises a 5′-homologous region (5′-HR) polynucleotide and a 3′-homologous region (3′-HR) polynucleotide, and wherein the replacement nucleic acid comprises, in 5′ to 3′ direction,
(i) the 5′-HR polynucleotide;
(ii) the internal polynucleotide; and
(iii) the 3′-HR polynucleotide.

3. The method of claim 1, wherein the target sequence or an additional target sequence or both are not present in the replacement nucleic acid.

4. The method of claim 1, wherein the SDN comprises an organelle targeting amino acid sequence (OTS), and wherein the OTS is a mitochondrion targeting amino acid sequence (MTS), a plastid targeting amino acid sequence (PTS), or an amino acid sequence that targets the mitochondrion and the plastid.

5. The method of claim 4, wherein the OTS is the MTS, and wherein the MTS comprises an amino acid sequence that is at least about 80% identical to an amino acid sequence of SEQ ID NO: 42, SEQ ID NO: 36, or SEQ ID NO: 129.

6. The method of claim 4, wherein the OTS is the PTS, and wherein the PTS comprises an amino acid sequence that is at least about 80% identical to an amino acid sequence of SEQ ID NO: 88.

7. The method of claim 1, wherein the SDN is selected from the group consisting of: a polynucleotide guided nuclease, a transcription activator-like effector nuclease (TALEN), a zinc-finger nuclease, a meganuclease, a restriction enzyme, and any combination thereof.

8. The method of claim 7, wherein the SDNS is the TALEN, wherein the TALEN comprises a first polypeptide and a second polypeptide, and wherein each of the first polypeptide and the second polypeptide comprise a nuclease domain.

9. The method of claim 8, wherein the nuclease domain comprises an amino acid sequence that is at least about 80% identical to an amino sequence of SEQ ID NO: 3, SEQ ID NO: 5, or SEQ ID NO: 60.

10. The method of claim 1, wherein the SDN comprises a single-chain TALEN comprising two copies of a nuclease domain.

11. The method of claim 10, wherein the nuclease domain comprises an amino acid sequence that is at least about 80% identical to an amino sequence of SEQ ID NO: 3, SEQ ID NO: 5, or SEQ ID NO: 60.

12. The method of claim 1, wherein the internal polynucleotide encodes a selectable marker polypeptide that provides resistance to an herbicide, and wherein the method further comprises introducing into a nucleus of the cell an additional polynucleotide encoding an additional selectable marker polypeptide, wherein the additional selectable marker polypeptide provides tolerance to an additional selective agent, and selecting a cell that grows in the presence of the additional selective agent.

13. The method of claim 12, wherein the cell is grown (a) simultaneously in a presence of the additional selective agent and in a presence of the herbicide, (b) sequentially first in a presence of the additional selective agent and subsequently in a presence of the herbicide, or (c) sequentially first in a presence of the herbicide and subsequently in a presence of the additional selective agent, wherein the additional selective agent and the herbicide are each present at an effective concentration.

14. The method of claim 12, wherein the additional selectable marker polypeptide is a polypeptide with hygromycin phosphotransferase (HPT) activity, and wherein the additional selective agent is hygromycin.

15. The method of claim 1, wherein the internal polynucleotide encodes a polypeptide selected from the group consisting of a cytoplasmic male sterility (CMS) polypeptide, a polypeptide with an herbicide-resistant enzyme activity, a polypeptide with an antibiotic-resistance enzyme activity, a polypeptide with phosphite dehydrogenase activity, and any combination thereof.

16. The method of claim 15, wherein the internal polynucleotide encodes the CMS polypeptide, wherein the CMS polypeptide comprises a rice orf79 polypeptide, a rice WA352 polypeptide, or both, and wherein the plant cell is a rice cell, and wherein the CMS polypeptide comprises a sequence having at least about 80% sequence identity to SEQ ID NO: 8 or SEQ ID NO: 128.

17. The method of claim 15, wherein the internal polynucleotide encodes the CMS polypeptide, wherein the CMS polypeptide comprises a wheat orf256 polypeptide, a wheat orf279 polypeptide, or both, and wherein the plant cell is a wheat cell, and wherein the CMS polypeptide comprises a sequence having at least about 80% sequence identity to SEQ ID NO: 9 or SEQ ID NO: 10.

18. A plant comprising a cell having an altered mitochondrion genome or an altered plastid genome, wherein the altered mitochondrion genome or the altered plastid genome comprises a heterologous polynucleotide comprising an internal polynucleotide in place of an endogenous polynucleotide,
wherein the plant comprises a polynucleotide encoding a site-directed nuclease (SDN), wherein the SDN binds to a target sequence present in the endogenous polynucleotide, and
wherein at least about 50% of mitochondrial genomes of a plurality of mitochondrial genomes or at least about 50% of the plastid genomes of a plurality of plastid genomes in the cell comprise the internal polynucleotide.

19. The plant of claim 18, wherein the SDN is selected from the group consisting of: a polynucleotide guided nuclease, a TALEN, a zinc-finger nuclease, a meganuclease, a restriction enzyme, and any combination thereof.

20. A polypeptide comprising a transcription activator-like (TAL) effector DNA-binding domain and a functional nuclease domain, wherein the functional nuclease domain comprises or consists of an amino acid sequence that is at least about 70% identical to an amino acid sequence of SEQ ID NO: 5 or SEQ ID NO: 60.


SEQUENCE LISTING (129 sequences)

SEQ ID NO: 1 - protein, 9 aa, synthetic construct
LAGLIDADG

SEQ ID NO: 2 - protein, 583 aa, Planomicrobium okeanokoites
MFLSMVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 3 - protein, 196 aa, Planomicrobium okeanokoites
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 4 - protein, 362 aa, Lysobacter sp.
MNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEEVTMLIRLSHADTARINATPGTSAGLGSQYFWLSKDCYDFFPPLTIRNQRGLRATYSCLISMLYVDLNVTDDNCRVTFEAENNLDFRLGTGPLRYTRLAAAGDLAAVTRLGENNYELRIYRAGAAEYQNLMPYAVNLIGHQGKQYGYISNAEFSAITGTRFRLRR

SEQ ID NO: 5 - protein, 161 aa, Lysobacter sp.
MNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLL

SEQ ID NO: 6 - protein, 393 aa, synthetic construct
MFKQASRLLSRSVAAASSKSVTTRAFSTELPSTLDSPPTKRFRIQAKNIFLTYPQCSLSKEEALEQIQRIQLSSNKKYIKIARELHEDGQPHLHVLLQLEGKVQITNIRLFDLVSPTRSAHFHPNIQRAKSSSDVKSYVDKDGDTIEWGEFQIDGRSARGGQQTANDSYAKALNATSLDQALQILKEEQPKDYFLQHHNLLNNAQKIFQRPPDPWTPLFPLSSFTNVPEEMQEWADAYFGVDAAARPLRYNSIIVEGDSRTGKTMWARSLGAHNYITGHLDFSPRTYYDEVEYNVIDDVDPTYLKMKHWKHLIGAQKEWQTNLKYGKPRVIKGGIPCIILCNPGPESSYQQFLEKPENEALKSWTLHNSTFCKLQGPLFNNQAAASSQGDSTL

SEQ ID NO: 7 - DNA, 201 bp, Beet curly top virus
  source 1..201: mol_type = other DNA
tgtactccgatgacgtggcttagcatattaacatatctattggagtattggagtattatatatattagtacaactttcataagggccatccgttataatattaccggatggcccgaaaaaaatgggcacccaatcaaaacgtgacacgtggaaggggactgttgaatgatgtgacgtttttgagcgggaaacttcctgaag

SEQ ID NO: 8 - protein, 79 aa, Oryza sativa
MANLVRWLFSTTRGTNGLPYFIFGVVVGGALLFALLKYQAPLYDPALMEKIIDHNIKAGHPIEVDYSWWGTSIRVVFPK

SEQ ID NO: 9 - protein, 256 aa, Triticum aestivum
MTNMVRWLFSTSRFTAFYLHFCIKFPYIYDFLLFSICLFFFLVRFISIILLPICNLFGASFIITLPPEIQDPQALAHLAGLNFYLSLYEQDPGWVTFIQNELNHNTPLEDIPGRLKLFLMEEKLSSMRQDVIQEFVALYQRIGPYLPIEPYLVDEALRSYLDHIHATDSFTVLQASYQDLRENEGGSVFFRDAVSHNRDLLEAESSARRCLEVEQRIRWEEIPKSKASLERAEHEHALDLFKSEDLRRELEKKRAG

SEQ ID NO: 10 - protein, 279 aa, Triticum aestivum
MPQLDKLTYFSQFFWLCLLLFTFYILLFNNNNGILGISRILKLRNQLLSHRGGEIRSKDPKNLEDISRKGFSTGLSYMYSSLSEVSQWCKTVDYLGNKISSSIFLYYLRGVLCPICLLFFKFLISFAFTCALIYVFKGGGFVAMAATNGASSSFLESSGEIDALLQTTTTTRTPENADYEDDNTSVNQEFLEDGQRARQAKLRELERLILQQYKDFIRQKYPWIPKGDILLPSMKGGVVEDVMEKLELETYSSSDLTDWINHLRANPKTLNFIFKDFVA

SEQ ID NO: 11 - protein, 631 aa, synthetic construct
MAATAKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVY

SEQ ID NO: 12 - DNA, 1896 bp, synthetic construct
  source 1..1896: mol_type = other DNA
atggctgcgactgccaagaccggccgtaagaaccatcaacgacatcatgtccttcccgctcgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaagcttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgctcgagactccagggccatatttgttggatatcatcgtcccacatcaagagcatgtgctgcctatgatcccaattgggggcgcattcaaggacatgatcctggatggtgatggcaggactgtgtattaa

SEQ ID NO: 13 - protein, 465 aa, synthetic construct
MFKQASRLLSRSVAAASSKSVTTRAFSTELPSTLDSGAAAPSPATGGVAPVPPSPRGSIIKRHTLSVFVGDESGMINRIAGVFARRGYNIESLAVGLNKDKALFTIVVSGTEKILKQVVEQLNKLVNVIQVDDLSKEPQVERELMLIKLNVEPDKRPEVMGLVDIFRAKVVDLSDHTLTIEVTGDPGKIVAVQRNLSKFGIKEIARTGKIALRREKMGESAPFWRFSAASYPDLEVAMPSKSHVNTAMKTANQNSEESSQGDVYPVESYENFTTNQILDAHWGVMADGDPTGLCSHSLSILVNDFPGVLNVVTGVFSRRGYNIQSLAVGPAEKEGTSRITTVVPGTDESIAKLVHQLYKLIDVYEVQDLTHLPFAARELMIIKIAVNTTARRAILDIADIFRAKTVDVSDHTVTLQLTGDLDKMVALQRMLEPYGICEVARTGRVALRRESGVDSKYLRGFSLPL

SEQ ID NO: 14 - protein, 445 aa, synthetic construct
AKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGIAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 15 - DNA, 1338 bp, synthetic construct
  source 1..1338: mol_type = other DNA
gcgaaggcggaggagatcgtgctccaacccatcagagagatttccggggcggttcaactgccagggtccaagtcgctctccaacagaatcctcctcctctccgccctctccgagggcacaactgtggtggacaacttgctgaacagtgaggatgttcattatatgcttgaggccctgaaagccctcgggctctctgtggaagcagataaagttgcaaagagagctgtagtcgttggctgtggtggcaagtttcctgttgagaaggatgcgaaagaggaagtgcaactcttcttggggaacgctggaattgcaatgcgatcattgactgctgccgtgactgctgctggtggaaatgcaacttatgtgcttgatggagtgccacgaatgagagagcgtcctattggtgatttggttgtcgggttgaaacaacttggtgcggatgttgactgtttccttggcactgaatgcccacctgttcgtgtcaagggaattggaggacttcctggtggcaaggttaagctctctggttccatcagcagtcaatatttgagtgccttgctgatggctgctcctttggcccttggggatgtggagatcgaaatcattgacaaactaatctccattccttatgttgaaatgactttgagattgatggagcgttttggtgtgaaggcagagcattctgatagttgggacagattctatattaagggagggcaaaagtataaatctcctggaaatgcctatgttgaaggtgatgcctcaagcgcgagctatttcttggctggtgctgcaatcactggaggcactgtgacagttcaaggttgtggtacaaccagtttgcaaggtgatgtcaaatttgctgaggtacttgagatgatgggagcaaaggttacatggactgacaccagtgtaacagtaactggtccacctagagagccttatgggaagaaacatctgaaagctgttgatgtcaacatgaacaaaatgcctgatgttgccatgacccttgccgttgttgcactcttcgctgatggtccaactgctatcagagatgtggcttcctggagagtaaaggaaactgaaagaatggttgcaattcgaactgagctaacaaaacttggagcatcggttgaagaaggtcctgactattgcatcatcactccacctgagaagctgaacattactgcaatcgacacttatgatgaccatagaatggctatggccttctccctcgctgcctgcgccgacgtgcccgtgactatccgtgaccctggttgcactagaaagacattccccaactatttcgacgttctaagcactttcgtcagaaactga

SEQ ID NO: 16 - protein, 370 aa, synthetic construct
MSSSLLTDLVNLDLSESTDKVIAEYIWVGGTGMDVRSKARTLSGPVDDPSKLPKWNFDGSGTGQATGDDSEVILHPQAIFRDPFRKGKNILVMCDCYAPNGEPIPTNNRYNAARIFSHPDVKAEEPWYGIEQEYTLLQKHINWPLGWPLGGYPGPQGPYYCAAGADKSYGRDIVDAHYKACLFAGINISGINAEVMPGQWEFQIGPVVGVSAGDHVWVARYILERITEIAGVVVSFDPKPIPGDWNGAGAHTNYSTKSMRSNGGYEVIKKAIKKLGMRHREHIAAYGDGNERRLTGRHETADINNFVWGVANRGASVRVGRDTEKDGKGYFEDRRPASNMDPYLVTAMIAETTILWEPSHGHGHGQSNGK

SEQ ID NO: 17 - DNA, 1110 bp, synthetic construct
  source 1..1110: mol_type = other DNA
tcgtcgtccctgctcactgacctcgttaacctcgacctgtcggagagcactgacaaggtcatcgccgagtatatatgggttggtggtactgggatggatgtgagaagcaaagccagaacattgtctggacctgttgatgacccaagcaagctaccaaagtggaactttgatggctccggcactggtcaagctactggtgacgacagtgaagttatcctccatcctcaagccatcttccgtgacccattcagaaaggggaagaacatcctggtcatgtgtgactgttatgcgcctaatggcgagccaattcctactaacaaccgatataatgcagcaagaatcttcagtcatcctgatgtcaaggctgaagaaccttggtatgggattgagcaagagtatacccttcttcaaaagcatatcaactggcctcttggctggccactaggtggctatccaggccctcaaggtccatattattgtgcggcgggagccgataaatcgtatgggcgtgacatcgttgatgcccattataaggcctgcctgtttgccggcatcaacatcagcgggatcaacgcagaagtcatgcctgggcaatgggagttccaaattggccctgtcgttggcgtatccgcaggggatcatgtctgggtggcacgttatattcttgagagaatcactgagattgctggcgtcgtcgtgtccttcgaccccaagcccattcctggagactggaatggtgccggtgctcataccaattatagcaccaagtcgatgagaagcaatggcggctacgaggtgatcaagaaagcgatcaagaagctcggtatgcgccatcgtgagcatatcgccgcctatggcgacggcaacgagcgtagactcactggtcgtcatgagactgccgacatcaacaacttcgtctggggcgtagcgaaccgtggcgcgtcggtgcgtgtcggtcgagacaccgagaaggacggcaaaggttacttcgaggacagacgaccagcgtccaacatggacccatatctggtgaccgccatgatcgccgagactaccatcctctgggagcccagtcatggtcatggacatggccaatccaacggcaagtga

SEQ ID NO: 18 - DNA, 41 bp, Oryza sativa
  source 1..41: mol_type = other DNA
gaatttctctgacattccatgtttccgaaacggatcctata

SEQ ID NO: 19 - DNA, 40 bp, Oryza sativa
  source 1..40: mol_type = other DNA
tgaacagtcactcacttttgacagttatacgattccagaa

SEQ ID NO: 20 - DNA, 75 bp, synthetic construct
  source 1..75: mol_type = other DNA
gaatttctctgtgaacagtcactcacttttgacagttatacgattccagaagatgatccagaattggatcctata

SEQ ID NO: 21 - protein, 36 aa, synthetic construct
DEVDFQGPCNDSSDPLVVAASIIGILHLILWILDRL

SEQ ID NO: 22 - protein, 4 aa, synthetic construct
DEVD

SEQ ID NO: 23 - RNA, 27 nt, Triticum aestivum
  source 1..27: mol_type = other RNA
acttttgacagttatacgattccagaa

SEQ ID NO: 24 - RNA, 27 nt, Triticum aestivum
  source 1..27: mol_type = other RNA
tgggctgtaccttcctcaggtgtcaaa

SEQ ID NO: 25 - RNA, 27 nt, Triticum aestivum
  source 1..27: mol_type = other RNA
gctgtacctggtcgttcaaatcttacc

SEQ ID NO: 26 - DNA, 6836 bp, synthetic construct
  source 1..6836: mol_type = other DNA
gggcccaaattcaattgtatatgagctcatatacaagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaattagggataacagggtaataatcacaagtgagaaccacaggtagcaataggtattacagaaatttcctcgagtctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaatacgactcactatagtaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagttgaacagtcactcacttttgacagttatacgattccagaaaagaccggccgtaagaaccatcaacgacatcatgtccttcccgcacgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaaacttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgcttgagactccagggccatatttgttggatatcatcgtcccacatcaagagcatgtgctgcctatgatcccaattgggggcgcattcaaggacatgatcctggatggtgatggcaggactgtgtattaatgaggtaccaagcgatcgcaaacctaggaaaagatctagacgaggtgtagcgcagtctggtcagcgcatctgttttgggtacagagggccataggttcgaatcctgtcaccttgagtctggccccaaattctaatttctactgttgtagatttcaattatgaaattactcatgtccctttcgtccagtggttaggacatcgtcttttcatgtcgaagacacgggttcgattcccgtaagggatagtctggccccaaattctaatttctactgttgtagatgtcacatttctaccggtgcacattccagcttatttgatacccacttcaagtttctatcaaaccatgtctttttcttcgaacgtcaatctcgtaggaaagatctaaaaagcttaagcggccgcaaaaaccccttggggcctctaaacgggtcttgaggggttttttgtagatagacctttttatttttcgtcattcgatcacgaaaaggccggccaaacctcgaggtaattaaagcggccgaaattagctagcaaataagcatgcaatcacaagtgagaaccacaggtagcaataggtattacagtagggataacagggtaatgaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaaggcgcgcc

SEQ ID NO: 27 - DNA, 774 bp, synthetic construct
  source 1..774: mol_type = other DNA
aaccccttggggcctctaaacgggtcttgaggggttttttgtagatagacctttttatttttcgtcattcgatcacgaaaacagggattctggaacggccaagaatcccagcggttgttcgggtcgaaaaaccgagaacaagacatgccacaaagtggcagatgaaggcaggggggagagcctagtcctcaacctcttcttccccaaaaggtagttatgaacgtgccaaacttattggatttattcttggaatgctcataaccacctttactctttttttcattctttactcagaggaagccatgccgtttggagaagagcaccaagtggggggagtgtggagtccccgaaagaggagctttctaaaggcaagagaaaagctccgatggagcccttggagctacagggaccaccaacccttcgcagcttggacgatttgattcttgtgccactcagccctgaggagggcgcctgctcgacccagtcaggtactactccgccgccggccccgagtaactctgcgggggtcggtgcagccctttcttctattccggagtgcataaacaaggatcctcaaaaagcgaaatcatttcgttaatggcatttcagaaatgagtcataggcgcctgtacaatgacagaatagagagtcctttttttccagaatgaatcattctattcaaatctcacaagttctctttacgcgtcttctaggggcattgttgaacgcaatctgcaggaacaagaaatgattctttcttattttgaaacagaa

SEQ ID NO: 28 - DNA, 81 bp, synthetic construct
  source 1..81: mol_type = other DNA
aaccccttggggcctctaaacgggtcttgaggggttttttgtagatagacctttttatttttcgtcattcgatcacgaaaa

SEQ ID NO: 29 - DNA, 457 bp, Oryza sativa
  source 1..457: mol_type = other DNA
caaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaat

SEQ ID NO: 30 - DNA, 352 bp, synthetic construct
  source 1..352: mol_type = other DNA
agatctagacgaggtgtagcgcagtctggtcagcgcatctgttttgggtacagagggccataggttcgaatcctgtcaccttgagtctggccccaaattctaatttctactgttgtagatttcaattatgaaattactcatgtccctttcgtccagtggttaggacatcgtcttttcatgtcgaagacacgggttcgattcccgtaagggatagtctggccccaaattctaatttctactgttgtagatgtcacatttctaccggtgcacattccagcttatttgatacccacttcaagtttctatcaaaccatgtctttttcttcgaacgtcaatctcgtaggaaagatct

SEQ ID NO: 31 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 32 - DNA, 1615 bp, synthetic construct
  source 1..1615: mol_type = other DNA
caagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcggga

SEQ ID NO: 33 - DNA, 1210 bp, synthetic construct
  source 1..1210: mol_type = other DNA
gaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaa

SEQ ID NO: 34 - DNA, 2757 bp, synthetic construct
  source 1..2757: mol_type = other DNA
atgtttaaacaagcttctcgtctcctctcccgatctgtcgccgccgcatcttccaaatcggtgacgactcgtgccttttcaacggaacttccatcgacgctcgattccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 35 - protein, 918 aa, synthetic construct
MFKQASRLLSRSVAAASSKSVTTRAFSTELPSTLDSNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 36 - protein, 36 aa, Arabidopsis thaliana
MFKQASRLLSRSVAAASSKSVTTRAFSTELPSTLDS

SEQ ID NO: 37 - DNA, 505 bp, Oryza sativa
  source 1..505: mol_type = other DNA
gaaaaggtcaagtctaagcgacatatggcacgaaaaggaaatccaatttcggtaatccgaagcccaccttaccttaatccattttcttgaggatcttgcatttgttcgttctctgccgacctattgtttccatcactcctgttgcttcttcctttctctatccaacccgagctctatcgcccatggctgaactcttctctgcagcctcatcgccaacaaaaggtccacatccttcaatcggcatgtactatggaatggctttaatgtttctatatccggaagtagggaatctcgtgcttgcatatctaaatctaagttttgagacagacctttcatgggttcaaagaaaagaagagtacgagtgggtgatgtgattgactaataggaaagtgataggagttgtgatcgggtttctttctttttttgccaaggatattcttattcactgctcagagaaaatgaaaaaaaacaaactaggattcttccgaactcaactccgtggcca

SEQ ID NO: 38 - DNA, 18 bp, Oryza sativa
  source 1..18: mol_type = other DNA
ctcatcgccaacaaaagg

SEQ ID NO: 39 - DNA, 18 bp, Oryza sativa
  source 1..18: mol_type = other DNA
ccatagtacatgccgatt

SEQ ID NO: 40 - DNA, 3195 bp, synthetic construct
  source 1..3195: mol_type = other DNA
atggcagcgaagatccggatcgtcatgaagtccttcatgagccaggccaataaggtggagggcgtcatcccgtatgcccaaaaagtcgggctcccagagtcgcgctctctctacacagtcttgcggtcgccacatatcgataaaaagtccagagaacaattctctatgaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaagcactcgagacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccacagcaggtcgtggcaattgctagcaacaacggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagccaactcgtaaagagcgaattggaggaaaagaaaagcgaattgaggcataaactaaaatatgtaccacatgaatatattgaactcattgaaattgctaggaactcaactcaagacaggattcttgaaatgaaagttatggaatttttcatgaaagtctatggttacagggggaaacatcttggtgggtcaaggaagccagatggggctatctacacggtcgggtcacctattgactacggagtaattgtggacaccaaagcatattctggtgggtataacctcccaatcgggcaagcagacgaaatgcaaaggtatgtagaagaaaatcaaactagaaataaacatattaatcccaatgaatggtggaaagtatatccaagctctgtaactgaatttaaattcttgttcgtatctggtcatttcaaaggaaactacaaagctcaacttactagactaaatcatattaccaactgcaacggtgccgttctcagcgtagaagaattgctcatcggtggggaaatgatcaaagctggcacgcttacgcttgaggaagtaaggagaaaattcaataacggagaaattaactttggcagctga

SEQ ID NO: 41 - protein, 1064 aa, synthetic construct
MAAKIRIVMKSFMSQANKVEGVIPYAQKVGLPESRSLYTVLRSPHIDKKSREQFSMTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPQQVVAIASNNGGRPALESIVAQLSRPDPALAALTNDHLVALACLGGRPALDAVKKGLPHAPALIKRTGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGS

SEQ ID NO: 42 - protein, 56 aa, Oryza sativa
MAAKIRIVMKSFMSQANKVEGVIPYAQKVGLPESRSLYTVLRSPHIDKKSREQFSM

SEQ ID NO: 43 - DNA, 3135 bp, synthetic construct
  source 1..3135: mol_type = other DNA
atgtttaaacaagcttcccgtcttctgtcacgctcagttgcagctgcttcctctaagtccgtcaccacccgcgctttctccaccgagctgccatcaaccctggactccaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaagcactcgagacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatcccagctggtgaaatctgagctggaggagaagaagtctgagctgagacacaagctgaagtacgtgcctcacgagtacatcgagctgatcgagatcgccagaaatagcacccaggatagaatcctggagatgaaggtgatggagttcttcatgaaggtgtacggctacagaggaaagcacctgggaggaagcagaaaacctgacggagccatttatacagtgggcagccctatcgattatggcgtgatcgtggatacaaaggcctacagcggaggctacaatctgcctattggacaggccgatgagatgcagagatacgtggaggagaaccagaccaggaacaagcacatcaaccctaacgagtggtggaaggtgtacccttctagcgtgaccgagttcaagttcctgtttgtgagcggccacttcaagggcaattataaggcccagctgaccaggctgaaccacatcacaaattgtaatggcgctgtgctgtctgtggaggaactgctgattggaggagagatgattaaggccggaacactgacactggaggaggtgagaagaaagttcaacaacggcgagatcaacttcggatcctga

SEQ ID NO: 44 - protein, 1044 aa, synthetic construct
MFKQASRLLSRSVAAASSKSVTTRAFSTELPSTLDSTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPQQVVAIASNGGGRPALESIVAQLSRPDPALAALTNDHLVALACLGGRPALDAVKKGLPHAPALIKRTGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGS

SEQ ID NO: 45 - DNA, 951 bp, synthetic construct
  source 1..951: mol_type = other DNA
atgtttaaacaagcttctcgtctcctctcccgatctgtcgccgccgcatcttccaaatcggtgacgactcgtgccttttcaacggaacttccatcgacgctcgattccggcgccgtgacggtgttcggcattctgaatctcaccgaggactccttcttcgatgagagccggcggctggaccccgccggcgctgtcaccgcggcgatcgaaatgctgcgggtcggatcagacgtcgtggatgtcggaccggccgccagccatccggacgcgaggcctgtgtcgccggccgatgagatcagacgcattgcgccgctcttggacgccctgtccgatcagatgcaccgcgtttcaatcgacagcttccaaccggaaacccagcgctatgcgctcaagcgcggcgtgggctacctgaacgatatccaaggatttcctgaccctgcgctctatcccgatattgctgaggcggattgcaggctggtggttatgcactcagcgcagcgggatggcatcgccacccgcaccggtcaccttcggcccgaagacgcgctcgacgagattgtgcggttcttcgaggcgcgggtttccgccttgcggcggagcggggtcgctgccgaccggctcatcctcgatccggggatgggattcttcttgagccccgcaccggaaacatcgctgcacgtgctgtcgaaccttcaaaagctgaagtcggcgttggggcttccgctgttggtctcggtgtcgcggaaatccttcttgggcgctaccgttggccttcctgtgaaggatctgggtccagcgagccttgcggcggaacttcacgcgatcggcaatggcgctgactacgtccgcacccacgcgcctggagacctgcggagcgcaatcaccatctcggaaaccctcgcgaaatttcgcagccgcgacgccagagaccgggggttggatcatgcctag

SEQ ID NO: 46 - protein, 316 aa, synthetic construct
MFKQASRLLSRSVAAASSKSVTTRAFSTELPSTLDSGAVTVFGILNLTEDSFFDESRRLDPAGAVTAAIEMLRVGSDVVDVGPAASHPDARPVSPADEIRRIAPLLDALSDQMHRVSIDSFQPETQRYALKRGVGYLNDIQGFPDPALYPDIAEADCRLVVMHSAQRDGIATRTGHLRPEDALDEIVRFFEARVSALRRSGVAADRLILDPGMGFFLSPAPETSLHVLSNLQKLKSALGLPLLVSVSRKSFLGATVGLPVKDLGPASLAAELHAIGNGADYVRTHAPGDLRSAITISETLAKFRSRDARDRGLDHA

SEQ ID NO: 47 - protein, 279 aa, Escherichia coli
MVTVFGILNLTEDSFFDESRRLDPAGAVTAAIEMLRVGSDVVDVGPAASHPDARPVSPADEIRRIAPLLDALSDQMHRVSIDSFQPETQRYALKRGVGYLNDIQGFPDPALYPDIAEADCRLVVMHSAQRDGIATRTGHLRPEDALDEIVRFFEARVSALRRSGVAADRLILDPGMGFFLSPAPETSLHVLSNLQKLKSALGLPLLVSVSRKSFLGATVGLPVKDLGPASLAAELHAIGNGADYVRTHAPGDLRSAITISETLAKFRSRDARDRGLDHA

SEQ ID NO: 48 - DNA, 645 bp, synthetic construct
  source 1..645: mol_type = other DNA
atggccgccaagatccgcatcgtgatgaaatcttttatgagccaagctaacaaagttgaaggggttattccatacgcgcagaaggttggattgcctgaatcacgatccttgtataccgtgctacgatcgcctcacatcgacaagaagtcgagggagcagttctcgatgacagtggcgtatattgccatcggcagcaatctggcctctccgctggagcaggtcaatgctgccctgaaagcattgggcgatatccctgaaagccacattcttaccgtttcttcgttttaccgcaccccaccgctgggaccgcaagatcaacccgattacttgaacgcagccgtggcgctggaaacctctcttgcacctgaagagctgctcaatcacacacagcgtattgaattgcagcaaggtcgcgtccgcaaagctgaacgctggggaccacgcacgctggatctcgacatcatgctgtttggtaatgaagtgatcaatactgaacgcctgaccgttccgcactacgatatgaagaatcgtggatttatgctgtggccgctgtttgaaatcgcgccggagttggtgtttcctgatggggagatgttgcgtcaaatcttgcatacaagagcatttgacaaattgaacaaatggtaa

SEQ ID NO: 49 - protein, 214 aa, synthetic construct
MAAKIRIVMKSFMSQANKVEGVIPYAQKVGLPESRSLYTVLRSPHIDKKSREQFSMTVAYIAIGSNLASPLEQVNAALKALGDIPESHILTVSSFYRTPPLGPQDQPDYLNAAVALETSLAPEELLNHTQRIELQQGRVRKAERWGPRTLDLDIMLFGNEVINTERLTVPHYDMKNRGFMLWPLFEIAPELVFPDGEMLRQILHTRAFDKLNKW

SEQ ID NO: 50 - DNA, 5943 bp, synthetic construct
  source 1..5943: mol_type = other DNA
agacatatatatctatctaaatttattgatatcaatatgaatgtggaaaatgctaaaataacttacattgtgaaatgaaggaagtaattttcttcacaaattttggcacttgcacttacggcttgtgaagcccgctcgatctgaagatttttcaaacgatgccattcaccacgtgccactttatatcaaaccttatttgtatccaagatttctggtactgggcattagcgatgtcaaacaaactcgacgtaaagttgagggacagagtgtgaacttattttattcccagcagagaacgcctccaaggtttatgggctttctggtccatcgcaacgacacggactttctacagggctgagattacgggcctaaatgggctcccccgtcttgtgctccgcgctgtttcaagttgggccaaaatggaactgggccgaatagtccggtgcggctaacggcatccacagccaagccaacggtgccgttctcccgctctcgcatttcaaaaatcgaaaataaaatcggcagggccgccgacgacgcaaccacccgttgggatccaatctactcctacgcaccgaacaactcatcgcacccaccgatatgtgggacccagctgtaacgtgggcccaccggcaagcgggacgtctgcatcggtcgcacctacaacgaggtggccaggtcacctactgctttttcttggagcgacgcgtgcactcgctaaccgcacacgaaggaagcctctggacccacctgtcagtgaaactaacgagaggcgggtatggacttcaaccgagtgcaaacgaaatggcctccttgcggtggatattatcagaaagctccctaacttattaaatattctcgatatgatccaaccacccgacctttgcaggcacgatgtgagatgcgagtgaattttgaagacgtcaaagggggtaaaacgaaaaactcccccatcccttcgcttccgctcctgagaggtccaggctgtctggccctataaaacgggcggagtgcggggatcgcaaaccctaagccgagacaacgcagagaaaggcgtcttcgtactcgcctctctccgcgcctccgcgcttttcctcctctcccctctctcccttctccgccgccgtcgcagcatcaacccaatccgccgccaagcttcatccaggtacggatccgcgtcccatctccctcaccccccgtgttcttcgtgcctgcttctgggtcagatctgggtggattcgcggttgttggatgtggggggctgtgtttatttgtcggtggatctggttgtctggatctgcgttttctctgtcgtagttagcggatctgatgaaatgtttagtgttcgtgtatactggtatggtggatctggtcctaggatgcgtggaatggatatatgtaggcgaattggaggatttattttgtgaattttgctgaaatgatagttctaaacactggatctgacctcgggatgctgttaaatgtggaaatcatggtcgatgctgtcatgaacatggtgttcttatggtagatctgagcaatgtatgtttcaaaattgtttgtcacatggaaatgctatggttctagatgcaatagaatgatacatgccgagatcccctctagttgatatgatagatcatgatgttttacagctatgtcatatgaatatgttcatttgttaccgatgtatttggatctacttaacatttccaaagcacgccgcgttctaattctagatctggtagtcatgtttgtacacgtcatccacctaatacaaatacatatgtctagtgtttggtgacactgcccgtcagatctgttttttccagatctgtggaacaaatactccatgcatgtatggtagttttgaaacgatcttgtatcttccattgttgtagtaacaactaaataaagtacaattgttcaattattgggaatcgtattttctgtagtgccgatgtacagcatattcatagatgtctatttaggaactcaaattttaaattgaggactagttatttattatgggtcagtcttttgaattgtgttatcttgctgtactgaaataataatgtaccactaaggcgctaacatgtattttgtcctttcaggctgactcgacggatcccggggggcaagagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaagatggcagcgaagatccggatcgtcatgaagtccttcatgagccaggccaataaggtggagggcgtcatcccgtatgcccaaaaagtcgggctcccagagtcgcgctctctctacacagtcttgcggtcgccacatatcgataaaaagtccagagaacaattctctatgaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaagcactcgagacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccacagcaggtcgtggcaattgctagcaacaacggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagccaactcgtaaagagcgaattggaggaaaagaaaagcgaattgaggcataaactaaaatatgtaccacatgaatatattgaactcattgaaattgctaggaactcaactcaagacaggattcttgaaatgaaagttatggaatttttcatgaaagtctatggttacagggggaaacatcttggtgggtcaaggaagccagatggggctatctacacggtcgggtcacctattgactacggagtaattgtggacaccaaagcatattctggtgggtataacctcccaatcgggcaagcagacgaaatgcaaaggtatgtagaagaaaatcaaactagaaataaacatattaatcccaatgaatggtggaaagtatatccaagctctgtaactgaatttaaattcttgttcgtatctggtcatttcaaaggaaactacaaagctcaacttactagactaaatcatattaccaactgcaacggtgccgttctcagcgtagaagaattgctcatcggtggggaaatgatcaaagctggcacgcttacgcttgaggaagtaaggagaaaattcaataacggagaaattaactttggcagctgagaattcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 51 - DNA, 5780 bp, synthetic construct
  source 1..5780: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgtttaaacaagcttcccgtcttctgtcacgctcagttgcagctgcttcctctaagtccgtcaccacccgcgctttctccaccgagctgccatcaaccctggactccaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaagcactcgagacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatcccagctggtgaaatctgagctggaggagaagaagtctgagctgagacacaagctgaagtacgtgcctcacgagtacatcgagctgatcgagatcgccagaaatagcacccaggatagaatcctggagatgaaggtgatggagttcttcatgaaggtgtacggctacagaggaaagcacctgggaggaagcagaaaacctgacggagccatttatacagtgggcagccctatcgattatggcgtgatcgtggatacaaaggcctacagcggaggctacaatctgcctattggacaggccgatgagatgcagagatacgtggaggagaaccagaccaggaacaagcacatcaaccctaacgagtggtggaaggtgtacccttctagcgtgaccgagttcaagttcctgtttgtgagcggccacttcaagggcaattataaggcccagctgaccaggctgaaccacatcacaaattgtaatggcgctgtgctgtctgtggaggaactgctgattggaggagagatgattaaggccggaacactgacactggaggaggtgagaagaaagttcaacaacggcgagatcaacttcggatcctgagaattcggtaccgagtttgaatcaaatcttcacttgtttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcggga

SEQ ID NO: 52 - DNA, 2449 bp, synthetic construct
  source 1..2449: mol_type = other DNA
tagctagcatactcgaggtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgatttggaaatgcatatctgtatttgagtcggtttttaagttcgttgcttttgtaaatacagagggatttgtataagaaatatctttaaaaaacccatatgctaatttgacataatttttgagaaaaatatatattcaggcgaattccacaatgaacaataataagattaaaatagcttgcccccgttgcagcgatgggtattttttctagtaaaataaaagataaacttagactcaaaacatttacaaaaacaacccctaaagtcctaaagcccaaagtgctatgcacgatccatagcaagcccagcccaacccaacccaacccaacccaccccagtgcagccaactggcaaatagtctccacccccggcactatcaccgtgagttgtccgcaccaccgcacgtctcgcagccaaaaaaaaaaaaagaaagaaaaaaaagaaaaagaaaaacagcaggtgggtccgggtcgtgggggccggaaaagcgaggaggatcgcgagcagcgacgaggcccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaagatgtttaaacaagcttctcgtctcctctcccgatctgtcgccgccgcatcttccaaatcggtgacgactcgtgccttttcaacggaacttccatcgacgctcgattccggcgccgtgacggtgttcggcattctgaatctcaccgaggactccttcttcgatgagagccggcggctggaccccgccggcgctgtcaccgcggcgatcgaaatgctgcgggtcggatcagacgtcgtggatgtcggaccggccgccagccatccggacgcgaggcctgtgtcgccggccgatgagatcagacgcattgcgccgctcttggacgccctgtccgatcagatgcaccgcgtttcaatcgacagcttccaaccggaaacccagcgctatgcgctcaagcgcggcgtgggctacctgaacgatatccaaggatttcctgaccctgcgctctatcccgatattgctgaggcggattgcaggctggtggttatgcactcagcgcagcgggatggcatcgccacccgcaccggtcaccttcggcccgaagacgcgctcgacgagattgtgcggttcttcgaggcgcgggtttccgccttgcggcggagcggggtcgctgccgaccggctcatcctcgatccggggatgggattcttcttgagccccgcaccggaaacatcgctgcacgtgctgtcgaaccttcaaaagctgaagtcggcgttggggcttccgctgttggtctcggtgtcgcggaaatccttcttgggcgctaccgttggccttcctgtgaaggatctgggtccagcgagccttgcggcggaacttcacgcgatcggcaatggcgctgactacgtccgcacccacgcgcctggagacctgcggagcgcaatcaccatctcggaaaccctcgcgaaatttcgcagccgcgacgccagagaccgggggttggatcatgcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 53 - DNA, 1688 bp, synthetic construct
  source 1..1688: mol_type = other DNA
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatggccgccaagatccgcatcgtgatgaaatcttttatgagccaagctaacaaagttgaaggggttattccatacgcgcagaaggttggattgcctgaatcacgatccttgtataccgtgctacgatcgcctcacatcgacaagaagtcgagggagcagttctcgatgacagtggcgtatattgccatcggcagcaatctggcctctccgctggagcaggtcaatgctgccctgaaagcattgggcgatatccctgaaagccacattcttaccgtttcttcgttttaccgcaccccaccgctgggaccgcaagatcaacccgattacttgaacgcagccgtggcgctggaaacctctcttgcacctgaagagctgctcaatcacacacagcgtattgaattgcagcaaggtcgcgtccgcaaagctgaacgctggggaccacgcacgctggatctcgacatcatgctgtttggtaatgaagtgatcaatactgaacgcctgaccgttccgcactacgatatgaagaatcgtggatttatgctgtggccgctgtttgaaatcgcgccggagttggtgtttcctgatggggagatgttgcgtcaaatcttgcatacaagagcatttgacaaattgaacaaatggtaaagcagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 54 - DNA, 24 bp, synthetic construct
  source 1..24: mol_type = other DNA
gctactggtgttgggcaacatcaa

SEQ ID NO: 55 - DNA, 25 bp, synthetic construct
  source 1..25: mol_type = other DNA
atatgtatgcgccctattcgcctta

SEQ ID NO: 56 - DNA, 20 bp, Oryza sativa
  source 1..20: mol_type = other DNA
tcggtaatccgaagcccacc

SEQ ID NO: 57 - DNA, 26 bp, Oryza sativa
  source 1..26: mol_type = other DNA
ccatagtacatgccgattgaaggatg

SEQ ID NO: 58 - DNA, 6837 bp, synthetic construct
  source 1..6837: mol_type = other DNA
gggcccaaattcaattgtatatgagctcatatacaagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaattagggataacagggtaataatcacaagtgagaaccacaggtagcaataggtattacagaaatttcctcgagtctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaatacgactcactatagtaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagtgaatttctctgacattccatgtttccgaaacggatcctataaagaccggccgtaagaaccatcaacgacatcatgtccttcccgcacgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaaacttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgcttgagactccagggccatatttgttggatatcatcgtcccacatcaagagcatgtgctgcctatgatcccaattgggggcgcattcaaggacatgatcctggatggtgatggcaggactgtgtattaatgaggtaccaagcgatcgcaaacctaggaaaagatctagacgaggtgtagcgcagtctggtcagcgcatctgttttgggtacagagggccataggttcgaatcctgtcaccttgagtctggccccaaattctaatttctactgttgtagatttcaattatgaaattactcatgtccctttcgtccagtggttaggacatcgtcttttcatgtcgaagacacgggttcgattcccgtaagggatagtctggccccaaattctaatttctactgttgtagatgtcacatttctaccggtgcacattccagcttatttgatacccacttcaagtttctatcaaaccatgtctttttcttcgaacgtcaatctcgtaggaaagatctaaaaagcttaagcggccgcaaaaaccccttggggcctctaaacgggtcttgaggggttttttgtagatagacctttttatttttcgtcattcgatcacgaaaaggccggccaaacctcgaggtaattaaagcggccgaaattagctagcaaataagcatgcaatcacaagtgagaaccacaggtagcaataggtattacagtagggataacagggtaatgaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaaggcgcgcc

SEQ ID NO: 59 - DNA, 1398 bp, synthetic construct
  source 1..1398: mol_type = other DNA
atgtttaaacaagcttctcgtctcctctcccgatctgtcgccgccgcatcttccaaatcggtgacgactcgtgccttttcaacggaacttccatcgacgctcgattccggcgccgccgctccctctccggccaccggcggcgtcgcgccggttccgccaagccccaggggctcgattataaagcgtcatacactatcagtttttgttggagatgaaagtgggatgattaatcgaattgctggagtttttgctagaagaggatacaacattgagtcattggcagttgggctgaacaaggacaaggcattatttacaatagtagtatctggaactgagaaaatactgaaacaggttgtagagcaactaaacaaacttgttaatgttatacaggttgacgatctgtcgaaggaaccacaagttgaaagagagctaatgcttataaaactaaatgttgagccagataagcgaccggaggtgatgggtttggttgacatcttcagagcaaaggtggttgacctttctgaccacacactaactattgaggtaactggagatcctggaaaaattgttgctgtacagaggaacctaagcaaatttggaatcaaagaaattgccagaactggcaagatagctttgcggcgtgagaaaatgggagaaagtgctcctttttggcggttctctgcagcttcctatcctgatcttgaagtggcaatgccatcaaaatctcatgtcaacactgcgatgaaaacagctaaccagaattctgaagaatcttcacaaggtgatgtctatccagtggagtcatatgaaaacttcacaacaaatcaaattcttgatgctcattggggtgtcatggctgacggtgatccaacagggctttgttcacattctttgtccattttggtgaatgatttccctggagttctcaatgttgtaacaggtgttttctcccgaagaggctacaatattcagagtctggctgttggcccagctgaaaaagaaggcacttctcggatcactactgttgtccctggaactgatgagtctattgccaagctagtacaccaactgtataagctcattgatgtttatgaggtccaggatcttactcatttaccatttgctgctagagagttaatgatcataaagattgctgtaaacaccacagcccgcagggctatcctagatattgctgatattttccgggccaaaactgtggatgtatcagatcacaccgtaactcttcagcttactggagaccttgataaaatggtcgcattacaaaggatgctcgagccctatggcatttgtgaggttgcacgaactggcagggttgcgttgcgccgtgagtcaggagtcgattccaaatacctccgtgggttttcccttcctctgtag

SEQ ID NO: 60 - protein, 198 aa, Lysobacter sp.
MNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 61 - protein, 37 aa, Lysobacter sp.
DQRWNYHRPTLGDPANPAWSLLYDEQAADIRLDETEE

SEQ ID NO: 62 - DNA, 18 bp, Oryza sativa
  source 1..18: mol_type = other DNA
gcagcctcatcgccaaca

SEQ ID NO: 63 - DNA, 3195 bp, synthetic construct
  source 1..3195: mol_type = other DNA
atggcagcgaagatccggatcgtcatgaagtccttcatgagccaggccaataaggtggagggcgtcatcccgtatgcccaaaaagtcgggctcccagagtcgcgctctctctacacagtcttgcggtcgccacatatcgataaaaagtccagagaacaattctctatgaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctcactcccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaagcactcgagacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccacagcaggtcgtggcaattgctagcaatatcggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagcatgaatgaattaaccttgcaccctaacgtctacgcaacaggagcctccaagggtttgatacacgtattggaaagggtgtgggtaagggagcatactccaggtgatggaacaatatttgtcattagcggattcggaaattataatggaggtgtaaggttctttgaatccttcagggagcataaacgcaggggaggtgaattggttgctgtctttgcaggatcaacctcggctaggttgacttctaaacaagtagtccatgaaatgttggactgtggtgcagacgtacatattgtaaatcgcaaaaggttgcttcatgcaaaatgctatggtacgtccaaaatgagcggagacagcttgatagtcacaagcggtaactttacagggcctggtatgagccaaaatgtcgagatgagcgtgctcttggatgctgacacgactgcggaaatgggatttagctggggtacactcttggaaaatctgttggaccaaaggtggaattatcacagaccgacgctcggcgaccctgcgaaccctgcatggtcgttgctgtacgacgaacaagctgcggacatacgcctcgacgagacagaggaatga

SEQ ID NO: 64 - protein, 1064 aa, synthetic construct
MAAKIRIVMKSFMSQANKVEGVIPYAQKVGLPESRSLYTVLRSPHIDKKSREQFSMTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPQQVVAIASNIGGRPALESIVAQLSRPDPALAALTNDHLVALACLGGRPALDAVKKGLPHAPALIKRTGSGSMNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 65 - DNA, 3135 bp, synthetic construct
  source 1..3135: mol_type = other DNA
atgtttaaacaagcttcccgtcttctgtcacgctcagttgcagctgcttcctctaagtccgtcaccacccgcgctttctccaccgagctgccatcaaccctggactccaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcangctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgttccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaagcactcgagacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatccatgaacgagttaacgctgcatccgaatgtgtatgcgaccggcgcgagtaaagggctgatacatgtgctggagcgcgtatgggttcgagaacacacgcctggggacggcacgatcttcgtgatatctggctttggcaactacaacggtggcgttcgcttcttcgagtcgtttcgtgaacacaagagacgtggcggggagctggtagcggtgttcgctggcagcacaagcgcgcgactgacaagcaaacaggttgttcacgagatgcttgattgcggggcggatgttcacatagtgaacagaaagcgactccttcacgctaagtgttacggcacaagcaagatgtcaggtgattccctaattgtaacctcaggcaatttcacaggtccgggaatgtcgcagaacgtggaaatgtctgtattgctagacgcagatacaaccgccgagatggggttctcgtggggcacgttgcttgagaacttgcttgatcagcgatggaactaccatcgccccaccttaggcgatccagcaaatccagcctggagcctgctttatgatgagcaggcagccgatattaggttggatgaaactgaagaatga

SEQ ID NO: 66 - protein, 1044 aa, synthetic construct
MFKQASRLLSRSVAAASSKSVTTRAFSTELPSTLDSTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCXAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLFQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPQQVVAIASNGGGRPALESIVAQLSRPDPALAALTNDHLVALACLGGRPALDAVKKGLPHAPALIKRTGSGSMNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 67 - DNA, 5943 bp, synthetic construct
  source 1..5943: mol_type = other DNA
agacatatatatctatctaaatttattgatatcaatatgaatgtggaaaatgctaaaataacttacattgtgaaatgaaggaagtaattttcttcacaaattttggcacttgcacttacggcttgtgaagcccgctcgatctgaagatttttcaaacgatgccattcaccacgtgccactttatatcaaaccttatttgtatccaagatttctggtactgggcattagcgatgtcaaacaaactcgacgtaaagttgagggacagagtgtgaacttattttattcccagcagagaacgcctccaaggtttatgggctttctggtccatcgcaacgacacggactttctacagggctgagattacgggcctaaatgggctcccccgtcttgtgctccgcgctgtttcaagttgggccaaaatggaactgggccgaatagtccggtgcggctaacggcatccacagccaagccaacggtgccgttctcccgctctcgcatttcaaaaatcgaaaataaaatcggcagggccgccgacgacgcaaccacccgttgggatccaatctactcctacgcaccgaacaactcatcgcacccaccgatatgtgggacccagctgtaacgtgggcccaccggcaagcgggacgtctgcatcggtcgcacctacaacgaggtggccaggtcacctactgctttttcttggagcgacgcgtgcactcgctaaccgcacacgaaggaagcctctggacccacctgtcagtgaaactaacgagaggcgggtatggacttcaaccgagtgcaaacgaaatggcctccttgcggtggatattatcagaaagctccctaacttattaaatattctcgatatgatccaaccacccgacctttgcaggcacgatgtgagatgcgagtgaattttgaagacgtcaaagggggtaaaacgaaaaactcccccatcccttcgcttccgctcctgagaggtccaggctgtctggccctataaaacgggcggagtgcggggatcgcaaaccctaagccgagacaacgcagagaaaggcgtcttcgtactcgcctctctccgcgcctccgcgcttttcctcctctcccctctctcccttctccgccgccgtcgcagcatcaacccaatccgccgccaagcttcatccaggtacggatccgcgtcccatctccctcaccccccgtgttcttcgtgcctgcttctgggtcagatctgggtggattcgcggttgttggatgtggggggctgtgtttatttgtcggtggatctggttgtctggatctgcgttttctctgtcgtagttagcggatctgatgaaatgtttagtgttcgtgtatactggtatggtggatctggtcctaggatgcgtggaatggatatatgtaggcgaattggaggatttattttgtgaattttgctgaaatgatagttctaaacactggatctgacctcgggatgctgttaaatgtggaaatcatggtcgatgctgtcatgaacatggtgttcttatggtagatctgagcaatgtatgtttcaaaattgtttgtcacatggaaatgctatggttctagatgcaatagaatgatacatgccgagatcccctctagttgatatgatagatcatgatgttttacagctatgtcatatgaatatgttcatttgttaccgatgtatttggatctacttaacatttccaaagcacgccgcgttctaattctagatctggtagtcatgtttgtacacgtcatccacctaatacaaatacatatgtctagtgtttggtgacactgcccgtcagatctgttttttccagatctgtggaacaaatactccatgcatgtatggtagttttgaaacgatcttgtatcttccattgttgtagtaacaactaaataaagtacaattgttcaattattgggaatcgtattttctgtagtgccgatgtacagcatattcatagatgtctatttaggaactcaaattttaaattgaggactagttatttattatgggtcagtcttttgaattgtgttatcttgctgtactgaaataataatgtaccactaaggcgctaacatgtattttgtcctttcaggctgactcgacggatcccggggggcaagagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaagatggcagcgaagatccggatcgtcatgaagtccttcatgagccaggccaataaggtggagggcgtcatcccgtatgcccaaaaagtcgggctcccagagtcgcgctctctctacacagtcttgcggtcgccacatatcgataaaaagtccagagaacaattctctatgaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctcactcccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaagcactcgagacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccacagcaggtcgtggcaattgctagcaatatcggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagcatgaatgaattaaccttgcaccctaacgtctacgcaacaggagcctccaagggtttgatacacgtattggaaagggtgtgggtaagggagcatactccaggtgatggaacaatatttgtcattagcggattcggaaattataatggaggtgtaaggttctttgaatccttcagggagcataaacgcaggggaggtgaattggttgctgtctttgcaggatcaacctcggctaggttgacttctaaacaagtagtccatgaaatgttggactgtggtgcagacgtacatattgtaaatcgcaaaaggttgcttcatgcaaaatgctatggtacgtccaaaatgagcggagacagcttgatagtcacaagcggtaactttacagggcctggtatgagccaaaatgtcgagatgagcgtgctcttggatgctgacacgactgcggaaatgggatttagctggggtacactcttggaaaatctgttggaccaaaggtggaattatcacagaccgacgctcggcgaccctgcgaaccctgcatggtcgttgctgtacgacgaacaagctgcggacatacgcctcgacgagacagaggaatgagaattcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 68 - DNA, 5780 bp, synthetic construct
  source 1..5780: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgtttaaacaagcttcccgtcttctgtcacgctcagttgcagctgcttcctctaagtccgtcaccacccgcgctttctccaccgagctgccatcaaccctggactccaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcangctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgttccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaagcactcgagacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatccatgaacgagttaacgctgcatccgaatgtgtatgcgaccggcgcgagtaaagggctgatacatgtgctggagcgcgtatgggttcgagaacacacgcctggggacggcacgatcttcgtgatatctggctttggcaactacaacggtggcgttcgcttcttcgagtcgtttcgtgaacacaagagacgtggcggggagctggtagcggtgttcgctggcagcacaagcgcgcgactgacaagcaaacaggttgttcacgagatgcttgattgcggggcggatgttcacatagtgaacagaaagcgactccttcacgctaagtgttacggcacaagcaagatgtcaggtgattccctaattgtaacctcaggcaatttcacaggtccgggaatgtcgcagaacgtggaaatgtctgtattgctagacgcagatacaaccgccgagatggggttctcgtggggcacgttgcttgagaacttgcttgatcagcgatggaactaccatcgccccaccttaggcgatccagcaaatccagcctggagcctgctttatgatgagcaggcagccgatattaggttggatgaaactgaagaatgagaattcggtaccgagtttgaatcaaatcttcacttgtttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcggga

SEQ ID NO: 69 - DNA, 6164 bp, synthetic construct
  source 1..6164: mol_type = other DNA
ccacctgtctctctttttttttctttgctttctcagatatttcataaaaaggactctctacggatccccaccaatgatcaattctcgcatgaatagctaaagatccagtaagtccaacgttgattccttcggacgtgtcaattggacaaatacgcccatagtgactcggatggatatctcggctccgaaaacttgcagttctccccgtcaatcctccaggacccaaacaactcacttttcgcccatgaaccgtttgtgtcaatggattggtttgatcaaaaacttgagataagggatatgtaccaaagaaggtctcgtaagtagttattaataaaatcgaggttgaagttggagttaccaaagtttgtggagtcggttttgattgacgtatgaatactctacggatagttttttgaaccgcatgttgtaaacgaccaagagccagtccgaattgatcttgtaacagatccgcaaccgaacgaatacgtttatttttcaagtgattcatatcgtcatcgtcaagtatacccgtttcaaatttcattccaatcaaatgatccgtagcggccaatacatctcgcggtaacaagaaggtgctgttctgaggtatatcaagattcagtcttcgattcatatttcgtcgaccaatccttcctaattcacatttttgttgaaaaaatttcttttgtaattcctcacataaggattccgaaaataccaggtccccacctacacaagcaaattgttgataaaactccaaaatagctttttcttttgactcaatcctcttcttctccttagcattagggaaagacaagaaaatttcagggtaggaaacattatctagaatttcttttagatttgaacccatagctgatgatagaactagaatagatatcttttgttttctactcacgcgagcccatatcctttcttttttatcaattgctaattccgatcttcctccccaatctgatattatagtcccggtgtagatagaaattcctttatggtctaattccgagcggtagtaaataccaggacttagcaatatttgattgatcacaattcggtatattccatttattataaaggttcctaaggaattcattataggaatgtttccaatagaaatggtttgcttttgcacatcgaaaccaaaaattaatcgcgcggatacgtataattcggaagaataggtgagtgattcatacacagcatccctttcttttatcgaaggttctagcaattgatatcctttcgcaaataattgaaatgaaatttcgtgatctggatctttaattgttggaaacttctcaagttcttctgccaagccttgattaatgaacctacaaaatccctcgaattggatctgactaaatccgggtattgtggacattccctcatttccattccggagcatcttaatcttaagtttcctgtttatcgaagaaaaattccattatcagctcactcttcatcaatccctacagatcgatctagcaattatggaatctatattctgtttactaaatcacatgaaattctcgggaactccacatacgtatttcatatatgtatttcatacatatgaatagagacaatttcaacgaaagttcgaatttcccaggggtccaaatggaatgaaaatgaagtgataaaacattcctagaacctcgaaaaaaattctgccacttacagtttatgatattcttagattggacggcaaagatttctcattttttctcctttctatgaatgggataaaaccaaaatataataatttataagcactagaaagtttgactttagttcgattgagaatagcacgcttagtttaatttcaaaaaagaatttgaaggttcttttttgtttcgtagtagtagaatttctataaaatataagatataagatttcgttgtagaatcctaaaataaagtaagtacttttttaagtacatgccaatctagttctggacctccacatagcaaagtcgaagagctcaacccaatgtgagttttttctattttgacttactccccccgccacgatcgaacgggaatggataagaggcttgtgggattgacgtgatagggtagggttggctatactgctggtggtaatacgtgtgcttgggagtccttgcaatttgaataaaccaagatcttaccatgaagaccggccgtaagaaccatcaacgacatcatgtccttcccgcacgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaaacttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgcttgagactccagggccatatttgttggatatcatcgtcccacatcaagagcatgtgctgcctatgatcccaattgggggcgcattcaaggacatgatcctggatggtgatggcaggactgtgtattaaggtacctttttctctaagcttggcttttctgctaacatatagcaatttttgaagaaaggaaagctagaaatacccaatatcttgctgaagcaagatattgggtatttcttttttttttattttgaatctttctattctgaattatccaaaaaaagcatgaatagattttttgaaaaagattggaaaaaagtaagtagaattagatttaagataaaagaaagttagttttagtaagaaacgtggatgaatacttgctcttttctcgattttagatcgtattttttggcggcatggccaagcggtaaggcaggggactgcaaatcctttatccccagttcaaatctgggtgccgcctgatcaataaaatacttaggcttatagtactgatccaaatcgacaaatttgcaccccgcataagttggggcaagagaataacaaggctcggcgatactggattttgagaacccaccgttctttcctcaaactggggtttgttttagcagtagtggcccaaaccgataccaatagggatgagatagcgtttccttattcttttattaataagaaagggctatgtatcatatttatacttaatgctccccaattctaccagaaatactctaagaatttgttaagagtaaattctttgaactgattcatccatagccttaggtcagaacccctttttgactctgtacccttgattctactatgattattcatcaatagtagaataattccttcatagaaataggagacataatttacatggatatagtaagtctcgcttgggctgctttaatggtagtctttacattttctctttcactagtagtatgggggaggagtggactctagggatactactaattgattgagtagtcgaattgttgtatcaactcttttctttaattgaccgttttgcattaatcattttgccgaactttattctttctctctttctttaattttgtttcactcctgtaagaaactcttgtaagaaagagtctttctattctttctatttctatttctatatagtagaatagaaatagaaagaatagaaaatagaaatagaaagagcggtgagagcaattcataatcgctactagaagcgacccatggttaaaaaagttctatacataagaactttagattttcttccgcggagctattcccaacatatcgcacattttccatttgttttatattcttttcttattcttagttcttaaaataaattttatgctcttttgaaacatttcgcgacatgtttaagcatgaaagattcgctggttttttccttttactttttttcttttactatagtctattctcatattatttttctctccctccatggattttttcattttgacttgattgaaattaagtggatgcagtgaaaaaagaaattgaattagactactatttcaatctagtaatctattctatgtagattcaagataatataatagaaagactctaaagtgtggtagaaaaaactatatgtagttttttctaccacactttatgattccaaccggatcctttcatttaagacttggatttttattttatttaatccctttttcatttcttcaatgattaattgaacttccaattaatcattttggctgactgtttttacataaataataagtaaaaaggcagtaggaactagaatgaacaatgcagtagcaataaatgcgagaatattgacttccataacctcttttttttcgcaataactcgggatgtaatcccatggagagggaaaagggtatcctgtaaattcaaggggaggacttgcattctgataatactgaatcaattcaatattatgaatatcggatctatcaaatcaattcatggttgagagtggaatagtataacataggaggatcccttccacgtatgatatgtatgtctttcgctatcaaccagaagtagtgataaaaaaattcctatactctcctctctttactgagaaacgcgaactaaaaaaagtgaaataggggtaccccataggtatttgatcttgcttcctgccccccttttttcatggaaaaaggaaagatgaatttctccattcattgaaccctagtt

SEQ ID NO: 70 - DNA, 116 bp, Oryza sativa
  source 1..116: mol_type = other DNA
aacccaatgtgagttttttctattttgacttactccccccgccacgatcgaacgggaatggataagaggcttgtgggattgacgtgatagggtagggttggctatactgctggtgg

SEQ ID NO: 71 - DNA, 51 bp, Oryza sativa
  source 1..51: mol_type = other DNA
taatacgtgtgcttgggagtccttgcaatttgaataaaccaagatcttacc

SEQ ID NO: 72 - DNA, 122 bp, Oryza sativa
  source 1..122: mol_type = other DNA
ggcttttctgctaacatatagcaatttttgaagaaaggaaagctagaaatacccaatatcttgctgaagcaagatattgggtatttcttttttttttattttgaatctttctattctgaatt

SEQ ID NO: 73 - DNA, 1998 bp, Oryza sativa
  source 1..1998: mol_type = other DNA
ccacctgtctctctttttttttctttgctttctcagatatttcataaaaaggactctctacggatccccaccaatgatcaattctcgcatgaatagctaaagatccagtaagtccaacgttgattccttcggacgtgtcaattggacaaatacgcccatagtgactcggatggatatctcggctccgaaaacttgcagttctccccgtcaatcctccaggacccaaacaactcacttttcgcccatgaaccgtttgtgtcaatggattggtttgatcaaaaacttgagataagggatatgtaccaaagaaggtctcgtaagtagttattaataaaatcgaggttgaagttggagttaccaaagtttgtggagtcggttttgattgacgtatgaatactctacggatagttttttgaaccgcatgttgtaaacgaccaagagccagtccgaattgatcttgtaacagatccgcaaccgaacgaatacgtttatttttcaagtgattcatatcgtcatcgtcaagtatacccgtttcaaatttcattccaatcaaatgatccgtagcggccaatacatctcgcggtaacaagaaggtgctgttctgaggtatatcaagattcagtcttcgattcatatttcgtcgaccaatccttcctaattcacatttttgttgaaaaaatttcttttgtaattcctcacataaggattccgaaaataccaggtccccacctacacaagcaaattgttgataaaactccaaaatagctttttcttttgactcaatcctcttcttctccttagcattagggaaagacaagaaaatttcagggtaggaaacattatctagaatttcttttagatttgaacccatagctgatgatagaactagaatagatatcttttgttttctactcacgcgagcccatatcctttcttttttatcaattgctaattccgatcttcctccccaatctgatattatagtcccggtgtagatagaaattcctttatggtctaattccgagcggtagtaaataccaggacttagcaatatttgattgatcacaattcggtatattccatttattataaaggttcctaaggaattcattataggaatgtttccaatagaaatggtttgcttttgcacatcgaaaccaaaaattaatcgcgcggatacgtataattcggaagaataggtgagtgattcatacacagcatccctttcttttatcgaaggttctagcaattgatatcctttcgcaaataattgaaatgaaatttcgtgatctggatctttaattgttggaaacttctcaagttcttctgccaagccttgattaatgaacctacaaaatccctcgaattggatctgactaaatccgggtattgtggacattccctcatttccattccggagcatcttaatcttaagtttcctgtttatcgaagaaaaattccattatcagctcactcttcatcaatccctacagatcgatctagcaattatggaatctatattctgtttactaaatcacatgaaattctcgggaactccacatacgtatttcatatatgtatttcatacatatgaatagagacaatttcaacgaaagttcgaatttcccaggggtccaaatggaatgaaaatgaagtgataaaacattcctagaacctcgaaaaaaattctgccacttacagtttatgatattcttagattggacggcaaagatttctcattttttctcctttctatgaatgggataaaaccaaaatataataatttataagcactagaaagtttgactttagttcgattgagaatagcacgcttagtttaatttcaaaaaagaatttgaaggttcttttttgtttcgtagtagtagaatttctataaaatataagatataagatttcgttgtagaatcctaaaataaagtaagtacttttttaagtacatgccaatctagttctggacctccacatagcaaagtcgaa

SEQ ID NO: 74 - DNA, 1966 bp, Oryza sativa
  source 1..1966: mol_type = other DNA
atccaaaaaaagcatgaatagattttttgaaaaagattggaaaaaagtaagtagaattagatttaagataaaagaaagttagttttagtaagaaacgtggatgaatacttgctcttttctcgattttagatcgtattttttggcggcatggccaagcggtaaggcaggggactgcaaatcctttatccccagttcaaatctgggtgccgcctgatcaataaaatacttaggcttatagtactgatccaaatcgacaaatttgcaccccgcataagttggggcaagagaataacaaggctcggcgatactggattttgagaacccaccgttctttcctcaaactggggtttgttttagcagtagtggcccaaaccgataccaatagggatgagatagcgtttccttattcttttattaataagaaagggctatgtatcatatttatacttaatgctccccaattctaccagaaatactctaagaatttgttaagagtaaattctttgaactgattcatccatagccttaggtcagaacccctttttgactctgtacccttgattctactatgattattcatcaatagtagaataattccttcatagaaataggagacataatttacatggatatagtaagtctcgcttgggctgctttaatggtagtctttacattttctctttcactagtagtatgggggaggagtggactctagggatactactaattgattgagtagtcgaattgttgtatcaactcttttctttaattgaccgttttgcattaatcattttgccgaactttattctttctctctttctttaattttgtttcactcctgtaagaaactcttgtaagaaagagtctttctattctttctatttctatttctatatagtagaatagaaatagaaagaatagaaaatagaaatagaaagagcggtgagagcaattcataatcgctactagaagcgacccatggttaaaaaagttctatacataagaactttagattttcttccgcggagctattcccaacatatcgcacattttccatttgttttatattcttttcttattcttagttcttaaaataaattttatgctcttttgaaacatttcgcgacatgtttaagcatgaaagattcgctggttttttccttttactttttttcttttactatagtctattctcatattatttttctctccctccatggattttttcattttgacttgattgaaattaagtggatgcagtgaaaaaagaaattgaattagactactatttcaatctagtaatctattctatgtagattcaagataatataatagaaagactctaaagtgtggtagaaaaaactatatgtagttttttctaccacactttatgattccaaccggatcctttcatttaagacttggatttttattttatttaatccctttttcatttcttcaatgattaattgaacttccaattaatcattttggctgactgtttttacataaataataagtaaaaaggcagtaggaactagaatgaacaatgcagtagcaataaatgcgagaatattgacttccataacctcttttttttcgcaataactcgggatgtaatcccatggagagggaaaagggtatcctgtaaattcaaggggaggacttgcattctgataatactgaatcaattcaatattatgaatatcggatctatcaaatcaattcatggttgagagtggaatagtataacataggaggatcccttccacgtatgatatgtatgtctttcgctatcaaccagaagtagtgataaaaaaattcctatactctcctctctttactgagaaacgcgaactaaaaaaagtgaaataggggtaccccataggtatttgatcttgcttcctgccccccttttttcatggaaaaaggaaagatgaatttctccattcattgaaccctagtt

SEQ ID NO: 75 - DNA, 2066 bp, synthetic construct
  source 1..2066: mol_type = other DNA
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 76 - DNA, 30 bp, Oryza sativa
  source 1..30: mol_type = other DNA
ctaacatgaatctgttcccatgcaatagtc

SEQ ID NO: 77 - DNA, 26 bp, synthetic construct
  source 1..26: mol_type = other DNA
acgtcaatcccacaagcctcttatcc

SEQ ID NO: 78 - DNA, 26 bp, Oryza sativa
  source 1..26: mol_type = other DNA
gcggggattgtagttcaattggtcag

SEQ ID NO: 79 - DNA, 27 bp, synthetic construct
  source 1..27: mol_type = other DNA
gatggcaggactgtgtattaaggtacc

SEQ ID NO: 80 - DNA, 160 bp, synthetic construct
  source 1..160: mol_type = other DNA
ttcctgaataccccgattcaaggacaaagaatttcctgctgctatcatataatactcatctctatttggtgataaataaaccacctgtctctctttttttttctttgctttctcagatatttcataaaaaggactctctacggatccccaccaatgatca

SEQ ID NO: 81 - DNA, 129 bp, synthetic construct
  source 1..129: mol_type = other DNA
ttgtagaatcctaaaataaagtaagtacttttttaagtacatgccaatctagttctggacctccacatagcaaagtcgaagagctcaacccaatgtgagttttttctattttgacttactccccccgcc

SEQ ID NO: 82 - DNA, 133 bp, synthetic construct
  source 1..133: mol_type = other DNA
ctagaaatacccaatatcttgctgaagcaagatattgggtatttcttttttttttattttgaatctttctattctgaattatccaaaaaaagcatgaatagattttttgaaaaagattggaaaaaagtaagta

SEQ ID NO: 83 - DNA, 110 bp, synthetic construct
  source 1..110: mol_type = other DNA
taggtatttgatcttgcttcctgccccccttttttcatggaaaaaggaaagatgaatttctccattcattgaaccctagttcgggactgacggggctcgaacccgcagct

SEQ ID NO: 84 - DNA, 18 bp, Oryza sativa
  source 1..18: mol_type = other DNA
ccgaacagcattattatc

SEQ ID NO: 85 - DNA, 18 bp, Oryza sativa
  source 1..18: mol_type = other DNA
ctccacagagatatgtac

SEQ ID NO: 86 - DNA, 3162 bp, synthetic construct
  source 1..3162: mol_type = other DNA
atggccccctccgtgatggcgtcgtcggccaccaccgtcgctcccttccagggctcaagtccaccgccggcttgccgtcgcccgccgtccgaactccagcttcggcaacgtcagcatggcggcaggatcagatgcaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcangctcacggcttgactccagaacaggtggtggctattgcttcccacgacggggggaaacaggccctgggaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggangaaaacngctctggaaacagtgcagcggctgctgcctgtgctgtgtcnnntcacggcttttgactccanaacaggtggtggctattgcttcccacnacggggggaaacaggccctggaaactgtgcagcncctgctgccagtgctgtgccangctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaagcactcgagacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccacagcaggtcgtggcaattgctagccacgacggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagcatgaatgaattaaccttgcaccctaacgtctacgcaacaggagcctccaagggtttgatacacgtattggaaagggtgtgggtaagggagcatactccaggtgatggaacaatatttgtcattagcggattcggaaattataatggaggtgtaaggttctttgaatccttcagggagcataaacgcaggggaggtgaattggttgctgtctttgcaggatcaacctcggctaggttgacttctaaacaagtagtccatgaaatgttggactgtggtgcagacgtacatattgtaaatcgcaaaaggttgcttcatgcaaaatgctatggtacgtccaaaatgagcggagacagcttgatagtcacaagcggtaactttacagggcctggtatgagccaaaatgtcgagatgagcgtgctcttggatgctgacacgactgcggaaatgggatttagctggggtacactcttggaaaatctgttggaccaaaggtggaattatcacagaccgacgctcggcgaccctgcgaaccctgcatggtcgttgctgtacgacgaacaagctgcggacatacgcctcgacgagacagaggaatga

SEQ ID NO: 87 - protein, 1061 aa, synthetic construct
MAPSVMASSATTVAPFQGSSPPPACRRPPSELQLRQRQHGGRIRCTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCXAHGLTPEQVVAIASHDGGKQALGTVQRLLPVLCQAHGLTPEQVVAIASNIGGKQALETVQRLLPVLCQAHGLTPEQVVAIASNNGXKXLWKQCSGCCLCCVXSRLLTPXQVVAIASHXGGKQALETVQXLLPVLCXAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPQQVVAIAXHDGXXPALXXIXXLLSIPDPALASRPDPALAALTNDHLVALACLGGRPALDAVKKGLPHAPALIKRTGSGSMNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 88 - protein, 45 aa, synthetic construct
MAPSVMASSATTVAPFQGSSPPPACRRPPSELQLRQRQHGGRIRC

SEQ ID NO: 89 - DNA, 3162 bp, synthetic construct
  source 1..3162: mol_type = other DNA
atggctcccagcgtcatggcctccagcgcgacaactgtggccccgtttcagggttcgagcccgccacctgcttgccgccggccacctagcgagctgcaactccgccagcgccaacacggagggagaattagatgcaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctcactcccgaacaggtggtggccattgccagcaacaacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaagcactcgagacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggcctgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccacagcaggtcgtggcaattgctagccacgacggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatccatgaacgagttaacgctgcatccgaatgtgtatgcgaccggcgcgagtaaagggctgatacatgtgctggagcgcgtatgggttcgagaacacacgcctggggacggcacgatcttcgtgatatctggctttggcaactacaacggtggcgttcgcttcttcgagtcgtttcgtgaacacaagagacgtggcggggagctggtagcggtgttcgctggcagcacaagcgcgcgactgacaagcaaacaggttgttcacgagatgcttgattgcggggcggatgttcacatagtgaacagaaagcgactccttcacgctaagtgttacggcacaagcaagatgtcaggtgattccctaattgtaacctcaggcaatttcacaggtccgggaatgtcgcagaacgtggaaatgtctgtattgctagacgcagatacaaccgccgagatggggttctcgtggggcacgttgcttgagaacttgcttgatcagcgatggaactaccatcgccccaccttaggcgatccagcaaatccagcctggagcctgctttatgatgagcaggcagccgatattaggttggatgaaactgaagaatga

SEQ ID NO: 90 - protein, 1053 aa, synthetic construct
MAPSVMASSATTVAPFQGSSPPPACRRPPSELQLRQRQHGGRIRCTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPQQVVAIASHDGGRPALESIVAQLSRPDPALAALTNDHLVALACLGGRPALDAVKKGLPHAPALIKRTGSGSMNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 91 - DNA, 5934 bp, synthetic construct
  source 1..5934: mol_type = other DNA
agacatatatatctatctaaatttattgatatcaatatgaatgtggaaaatgctaaaataacttacattgtgaaatgaaggaagtaattttcttcacaaattttggcacttgcacttacggcttgtgaagcccgctcgatctgaagatttttcaaacgatgccattcaccacgtgccactttatatcaaaccttatttgtatccaagatttctggtactgggcattagcgatgtcaaacaaactcgacgtaaagttgagggacagagtgtgaacttattttattcccagcagagaacgcctccaaggtttatgggctttctggtccatcgcaacgacacggactttctacagggctgagattacgggcctaaatgggctcccccgtcttgtgctccgcgctgtttcaagttgggccaaaatggaactgggccgaatagtccggtgcggctaacggcatccacagccaagccaacggtgccgttctcccgctctcgcatttcaaaaatcgaaaataaaatcggcagggccgccgacgacgcaaccacccgttgggatccaatctactcctacgcaccgaacaactcatcgcacccaccgatatgtgggacccagctgtaacgtgggcccaccggcaagcgggacgtctgcatcggtcgcacctacaacgaggtggccaggtcacctactgctttttcttggagcgacgcgtgcactcgctaaccgcacacgaaggaagcctctggacccacctgtcagtgaaactaacgagaggcgggtatggacttcaaccgagtgcaaacgaaatggcctccttgcggtggatattatcagaaagctccctaacttattaaatattctcgatatgatccaaccacccgacctttgcaggcacgatgtgagatgcgagtgaattttgaagacgtcaaagggggtaaaacgaaaaactcccccatcccttcgcttccgctcctgagaggtccaggctgtctggccctataaaacgggcggagtgcggggatcgcaaaccctaagccgagacaacgcagagaaaggcgtcttcgtactcgcctctctccgcgcctccgcgcttttcctcctctcccctctctcccttctccgccgccgtcgcagcatcaacccaatccgccgccaagcttcatccaggtacggatccgcgtcccatctccctcaccccccgtgttcttcgtgcctgcttctgggtcagatctgggtggattcgcggttgttggatgtggggggctgtgtttatttgtcggtggatctggttgtctggatctgcgttttctctgtcgtagttagcggatctgatgaaatgtttagtgttcgtgtatactggtatggtggatctggtcctaggatgcgtggaatggatatatgtaggcgaattggaggatttattttgtgaattttgctgaaatgatagttctaaacactggatctgacctcgggatgctgttaaatgtggaaatcatggtcgatgctgtcatgaacatggtgttcttatggtagatctgagcaatgtatgtttcaaaattgtttgtcacatggaaatgctatggttctagatgcaatagaatgatacatgccgagatcccctctagttgatatgatagatcatgatgttttacagctatgtcatatgaatatgttcatttgttaccgatgtatttggatctacttaacatttccaaagcacgccgcgttctaattctagatctggtagtcatgtttgtacacgtcatccacctaatacaaatacatatgtctagtgtttggtgacactgcccgtcagatctgttttttccagatctgtggaacaaatactccatgcatgtatggtagttttgaaacgatcttgtatcttccattgttgtagtaacaactaaataaagtacaattgttcaattattgggaatcgtattttctgtagtgccgatgtacagcatattcatagatgtctatttaggaactcaaattttaaattgaggactagttatttattatgggtcagtcttttgaattgtgttatcttgctgtactgaaataataatgtaccactaaggcgctaacatgtattttgtcctttcaggctgactcgacggatcccggggggcaagagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaagatggccccctccgtgatggcgtcgtcggccaccaccgtcgctcccttccagggctcaagtccaccgccggcttgccgtcgcccgccgtccgaactccagcttcggcaacgtcagcatggcggcaggatcagatgcaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcangctcacggcttgactccagaacaggtggtggctattgcttcccacgacggggggaaacaggccctgggaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggangaaaacngctctggaaacagtgcagcggctgctgcctgtgctgtgtcnnntcacggcttttgactccanaacaggtggtggctattgcttcccacnacggggggaaacaggccctggaaactgtgcagcncctgctgccagtgctgtgccangctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaagcactcgagacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccacaacaggtcgtggcaattgctanccacgacggcnganngcccgccctgganancattntggncctgctgtcnataccggacccagctctcgcttctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagcatgaatgaattaaccttgcaccctaacgtctacgcaacaggagcctccaagggtttgatacacgtattggaaagggtgtgggtaagggagcatactccaggtgatggaacaatatttgtcattagcggattcggaaattataatggaggtgtaaggttctttgaatccttcagggagcataaacgcaggggaggtgaattggttgctgtctttgcaggatcaacctcggctaggttgacttctaaacaagtagtccatgaaatgttggactgtggtgcagacgtacatattgtaaatcgcaaaaggttgcttcatgcaaaatgctatggtacgtccaaaatgagcggagacagcttgatagtcacaagcggtaactttacagggcctggtatgagccaaaatgtcgagatgagcgtgctcttggatgctgacacgactgcggaaatgggatttagctggggtacactcttggaaaatctgttggaccaaaggtggaattatcacagaccgacgctcggcgaccctgcgaaccctgcatggtcgttgctgtacgacgaacaagctgcggacatacgcctcgacgagacagaggaatgagaattcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 92 - DNA, 5807 bp, synthetic construct
  source 1..5807: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggctcccagcgtcatggcctccagcgcgacaactgtggccccgtttcagggttcgagcccgccacctgcttgccgccggccacctagcgagctgcaactccgccagcgccaacacggagggagaattagatgcaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctcactcccgaacaggtggtggccattgccagcaacaacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaagcactcgagacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggcctgacccccgaacaggtggtggccattgccagcaacggcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccacagcaggtcgtggcaattgctagccacgacggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatccatgaacgagttaacgctgcatccgaatgtgtatgcgaccggcgcgagtaaagggctgatacatgtgctggagcgcgtatgggttcgagaacacacgcctggggacggcacgatcttcgtgatatctggctttggcaactacaacggtggcgttcgcttcttcgagtcgtttcgtgaacacaagagacgtggcggggagctggtagcggtgttcgctggcagcacaagcgcgcgactgacaagcaaacaggttgttcacgagatgcttgattgcggggcggatgttcacatagtgaacagaaagcgactccttcacgctaagtgttacggcacaagcaagatgtcaggtgattccctaattgtaacctcaggcaatttcacaggtccgggaatgtcgcagaacgtggaaatgtctgtattgctagacgcagatacaaccgccgagatggggttctcgtggggcacgttgcttgagaacttgcttgatcagcgatggaactaccatcgccccaccttaggcgatccagcaaatccagcctggagcctgctttatgatgagcaggcagccgatattaggttggatgaaactgaagaatgagaattcggtaccgagtttgaatcaaatcttcacttgtttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcggga

SEQ ID NO: 93 - DNA, 368 bp, Oryza sativa
  source 1..368: mol_type = other DNA
ccagtttcgattattctcctcaaaggattctatcttaaaacacacattgctaacactagaaaacttcccttttctttctattgaaatgaaaaaggagaaaagtggatttctagtcacaagctacaaaattaagaacaaattggaaccattaactagaattctctttttattttgaatttcggtattctctcccgcctgccatttaatgtcataataaaagacaatggatttatgcctaatccgtatataggtaaactccaggtccgaacagcattattatctatggatcccccttatgtacatatctctgtggagaatcgttctttaatttttcattgcattaaatatcttgaataaaaaaaagaaaa

SEQ ID NO: 94 - DNA, 29 bp, Oryza sativa
  source 1..29: mol_type = other DNA
acacacattgctaacactagaaaacttcc

SEQ ID NO: 95 - DNA, 27 bp, Oryza sativa
  source 1..27: mol_type = other DNA
cggacctggagtttacctatatacgga

SEQ ID NO: 96 - protein, 630 aa, synthetic construct
MDPIKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVY

SEQ ID NO: 97 - protein, 630 aa, synthetic construct
MIPEKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVY

SEQ ID NO: 98 - protein, 627 aa, synthetic construct
MKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVY

SEQ ID NO: 99 - DNA, 18 bp, Oryza sativa
  source 1..18: mol_type = other DNA
tcggtaatccgaagccca

SEQ ID NO: 100 - DNA, 18 bp, Oryza sativa
  source 1..18: mol_type = other DNA
gcaagatcctcaagaaaa

SEQ ID NO: 101 - DNA, 3195 bp, synthetic construct
  source 1..3195: mol_type = other DNA
atggcagcgaagatccggatcgtcatgaagtccttcatgagccaggccaataaggtggagggcgtcatcccgtatgcccaaaaagtcgggctcccagagtcgcgctctctctacacagtcttgcggtcgccacatatcgataaaaagtccagagaacaattctctatgaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagccacgacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccacagcaggtcgtggcaattgctagcaatatcggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagcatgaatgaattaaccttgcaccctaacgtctacgcaacaggagcctccaagggtttgatacacgtattggaaagggtgtgggtaagggagcatactccaggtgatggaacaatatttgtcattagcggattcggaaattataatggaggtgtaaggttctttgaatccttcagggagcataaacgcaggggaggtgaattggttgctgtctttgcaggatcaacctcggctaggttgacttctaaacaagtagtccatgaaatgttggactgtggtgcagacgtacatattgtaaatcgcaaaaggttgcttcatgcaaaatgctatggtacgtccaaaatgagcggagacagcttgatagtcacaagcggtaactttacagggcctggtatgagccaaaatgtcgagatgagcgtgctcttggatgctgacacgactgcggaaatgggatttagctggggtacactcttggaaaatctgttggaccaaaggtggaattatcacagaccgacgctcggcgaccctgcgaaccctgcatggtcgttgctgtacgacgaacaagctgcggacatacgcctcgacgagacagaggaatga

SEQ ID NO: 102 - protein, 1064 aa, synthetic construct
MAAKIRIVMKSFMSQANKVEGVIPYAQKVGLPESRSLYTVLRSPHIDKKSREQFSMTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNIGGRPALESIVAQLSRPDPALAALTNDHLVALACLGGRPALDAVKKGLPHAPALIKRTGSGSMNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 103 - DNA, 5943 bp, synthetic construct
  source 1..5943: mol_type = other DNA
agacatatatatctatctaaatttattgatatcaatatgaatgtggaaaatgctaaaataacttacattgtgaaatgaaggaagtaattttcttcacaaattttggcacttgcacttacggcttgtgaagcccgctcgatctgaagatttttcaaacgatgccattcaccacgtgccactttatatcaaaccttatttgtatccaagatttctggtactgggcattagcgatgtcaaacaaactcgacgtaaagttgagggacagagtgtgaacttattttattcccagcagagaacgcctccaaggtttatgggctttctggtccatcgcaacgacacggactttctacagggctgagattacgggcctaaatgggctcccccgtcttgtgctccgcgctgtttcaagttgggccaaaatggaactgggccgaatagtccggtgcggctaacggcatccacagccaagccaacggtgccgttctcccgctctcgcatttcaaaaatcgaaaataaaatcggcagggccgccgacgacgcaaccacccgttgggatccaatctactcctacgcaccgaacaactcatcgcacccaccgatatgtgggacccagctgtaacgtgggcccaccggcaagcgggacgtctgcatcggtcgcacctacaacgaggtggccaggtcacctactgctttttcttggagcgacgcgtgcactcgctaaccgcacacgaaggaagcctctggacccacctgtcagtgaaactaacgagaggcgggtatggacttcaaccgagtgcaaacgaaatggcctccttgcggtggatattatcagaaagctccctaacttattaaatattctcgatatgatccaaccacccgacctttgcaggcacgatgtgagatgcgagtgaattttgaagacgtcaaagggggtaaaacgaaaaactcccccatcccttcgcttccgctcctgagaggtccaggctgtctggccctataaaacgggcggagtgcggggatcgcaaaccctaagccgagacaacgcagagaaaggcgtcttcgtactcgcctctctccgcgcctccgcgcttttcctcctctcccctctctcccttctccgccgccgtcgcagcatcaacccaatccgccgccaagcttcatccaggtacggatccgcgtcccatctccctcaccccccgtgttcttcgtgcctgcttctgggtcagatctgggtggattcgcggttgttggatgtggggggctgtgtttatttgtcggtggatctggttgtctggatctgcgttttctctgtcgtagttagcggatctgatgaaatgtttagtgttcgtgtatactggtatggtggatctggtcctaggatgcgtggaatggatatatgtaggcgaattggaggatttattttgtgaattttgctgaaatgatagttctaaacactggatctgacctcgggatgctgttaaatgtggaaatcatggtcgatgctgtcatgaacatggtgttcttatggtagatctgagcaatgtatgtttcaaaattgtttgtcacatggaaatgctatggttctagatgcaatagaatgatacatgccgagatcccctctagttgatatgatagatcatgatgttttacagctatgtcatatgaatatgttcatttgttaccgatgtatttggatctacttaacatttccaaagcacgccgcgttctaattctagatctggtagtcatgtttgtacacgtcatccacctaatacaaatacatatgtctagtgtttggtgacactgcccgtcagatctgttttttccagatctgtggaacaaatactccatgcatgtatggtagttttgaaacgatcttgtatcttccattgttgtagtaacaactaaataaagtacaattgttcaattattgggaatcgtattttctgtagtgccgatgtacagcatattcatagatgtctatttaggaactcaaattttaaattgaggactagttatttattatgggtcagtcttttgaattgtgttatcttgctgtactgaaataataatgtaccactaaggcgctaacatgtattttgtcctttcaggctgactcgacggatcccggggggcaagagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaagatggcagcgaagatccggatcgtcatgaagtccttcatgagccaggccaataaggtggagggcgtcatcccgtatgcccaaaaagtcgggctcccagagtcgcgctctctctacacagtcttgcggtcgccacatatcgataaaaagtccagagaacaattctctatgaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagccacgacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccacagcaggtcgtggcaattgctagcaatatcggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagcatgaatgaattaaccttgcaccctaacgtctacgcaacaggagcctccaagggtttgatacacgtattggaaagggtgtgggtaagggagcatactccaggtgatggaacaatatttgtcattagcggattcggaaattataatggaggtgtaaggttctttgaatccttcagggagcataaacgcaggggaggtgaattggttgctgtctttgcaggatcaacctcggctaggttgacttctaaacaagtagtccatgaaatgttggactgtggtgcagacgtacatattgtaaatcgcaaaaggttgcttcatgcaaaatgctatggtacgtccaaaatgagcggagacagcttgatagtcacaagcggtaactttacagggcctggtatgagccaaaatgtcgagatgagcgtgctcttggatgctgacacgactgcggaaatgggatttagctggggtacactcttggaaaatctgttggaccaaaggtggaattatcacagaccgacgctcggcgaccctgcgaaccctgcatggtcgttgctgtacgacgaacaagctgcggacatacgcctcgacgagacagaggaatgagaattcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 104 - DNA, 3135 bp, synthetic construct
  source 1..3135: mol_type = other DNA
atgtttaaacaagcttcccgtcttctgtcacgctcagttgcagctgcttcctctaagtccgtcaccacccgcgctttctccaccgagctgccatcaaccctggactccaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccacagcaggtcgtggcaattgctagcaatatcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatccatgaacgagttaacgctgcatccgaatgtgtatgcgaccggcgcgagtaaagggctgatacatgtgctggagcgcgtatgggttcgagaacacacgcctggggacggcacgatcttcgtgatatctggctttggcaactacaacggtggcgttcgcttcttcgagtcgtttcgtgaacacaagagacgtggcggggagctggtagcggtgttcgctggcagcacaagcgcgcgactgacaagcaaacaggttgttcacgagatgcttgattgcggggcggatgttcacatagtgaacagaaagcgactccttcacgctaagtgttacggcacaagcaagatgtcaggtgattccctaattgtaacctcaggcaatttcacaggtccgggaatgtcgcagaacgtggaaatgtctgtattgctagacgcagatacaaccgccgagatggggttctcgtggggcacgttgcttgagaacttgcttgatcagcgatggaactaccatcgccccaccttaggcgatccagcaaatccagcctggagcctgctttatgatgagcaggcagccgatattaggttggatgaaactgaagaatga

SEQ ID NO: 105 - protein, 1044 aa, synthetic construct
MFKQASRLLSRSVAAASSKSVTTRAFSTELPSTLDSTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPQQVVAIASNIGGRPALESIVAQLSRPDPALAALTNDHLVALACLGGRPALDAVKKGLPHAPALIKRTGSGSMNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 106 - DNA, 5784 bp, synthetic construct
  source 1..5784: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgtttaaacaagcttcccgtcttctgtcacgctcagttgcagctgcttcctctaagtccgtcaccacccgcgctttctccaccgagctgccatcaaccctggactccaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggcctcactcccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccacagcaggtcgtggcaattgctagcaatatcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatccatgaacgagttaacgctgcatccgaatgtgtatgcgaccggcgcgagtaaagggctgatacatgtgctggagcgcgtatgggttcgagaacacacgcctggggacggcacgatcttcgtgatatctggctttggcaactacaacggtggcgttcgcttcttcgagtcgtttcgtgaacacaagagacgtggcggggagctggtagcggtgttcgctggcagcacaagcgcgcgactgacaagcaaacaggttgttcacgagatgcttgattgcggggcggatgttcacatagtgaacagaaagcgactccttcacgctaagtgttacggcacaagcaagatgtcaggtgattccctaattgtaacctcaggcaatttcacaggtccgggaatgtcgcagaacgtggaaatgtctgtattgctagacgcagatacaaccgccgagatggggttctcgtggggcacgttgcttgagaacttgcttgatcagcgatggaactaccatcgccccaccttaggcgatccagcaaatccagcctggagcctgctttatgatgagcaggcagccgatattaggttggatgaaactgaagaatgagaattcggtaccgagtttgaatcaaatcttcacttgtttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcggga

SEQ ID NO: 107 - DNA, 6218 bp, synthetic construct
  source 1..6218: mol_type = other DNA
aagtcgaggtatttcgaattgtttgttgacacggacaaagtaagggaaaacccctgaaagaatttccatattgaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgattctttactgcgacagcatctagggctcctcgaataatgcgatatctcacaccgggtaaatccttaacccttcctcctcttactaatactacagaatgttcttgtaaattatggccaataccaggtatataagcagtgatttcaaatccagaggttaatcgtactctggcaactttacgtaaggcagagttgggttttttggggttgatagtggaaaagtcgacagataagtcacccttcctgtccctctacagaatcgtacatgagattttcacctaatacggctcctcgttcaattctttcgaagggatccttttcctcgttcgagagtcttcgccctttcttccactccgtcccgaagactaactaagaccaattgagtcacgttttcatgttctaattgaacactttccatttatgattaaaggagaagattgttcttttaccaaacatatgcggatcaaatcacgtcttataataagaagaaatctttctcggtatcaatccccttgcccctcattctttgagaatcagaaggatccttttcgagtttccatttcttcatttggaatctgggctcttctatcttcgacttattttttttttgctttattctttatttatttcatttcgatttttccctcttcctctatccctatcctctaggtacagcgtttgcatcaatagagaaccttttcctctgtatgaatctatattattccattccaatttcttcccgaaacttcccaagaaaaatcccgaattggatccaaaattgacgggttaatgtgagcttatccatgcggttaggcactcttcaaataggaatccattttctaactggctttcgtgctttggtgagtcgtccgagatcctttcgatgacctatgttgtgttgaagggatatctatatgatccgatcgattgcataagacccgcggtagcaatagaacggggaaagtatacagaaaagacagttcttttcaatttcgattatctatatattagttgtttctatttctagatatctatttctatatattagtattagttagtagtactattctattagttagcgatcccggctctgtgagttctttcttccgtgatgaactgtcggcaccagtcctccattttttctctgtggaccgaggagaaagggggctcagcaggaagaggattgtaccatgagagaagcacagaggtcaaccctcttcaaatatggaacatggattctgcaatgcaacgtagttgggtcctcatatcgatccgaatgaatcattctttctacagaggtcaatctttgcctattaggcaagaggatagcaagttcgaaattctgtctcggtaggacatggatttctattactatgaaattcataaatgaaatgaagtagttaatgggagggctaccattatcctttttcttgtatgtgttcctaagagaaggaatttgtccatttcatgtttcgaggtctcaaaaaagggcgtggaaacagataagaaactcttgaatggaaattgaaaagaaatgtagccccagttccttcggaaatggtaagatctttggcgcaagaagaaggggcgacccatatcatcttgacttggttctgcttcccctctttttttaagaataccgagtcgggttcttctcctaccagtatcgaatagaacatgctgaacaagatcttcttcatggaaacctgctcgatttagatcgggactagtttctttgagctcaacccaatgtgagttttttctattttgacttactccccccgccacgatcgaacgggaatggataagaggcttgtgggattgacgtgatagggtagggttggctatactgctggtggtaatacgtgtgcttgggagtccttgcaatttgaataaaccaagatcttaccatgaagaccggccgtaagaaccatcaacgacatcatgtccttcccgcacgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaaacttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgcttgagactccagggccatatttgttggatatcatcgtcccacatcaagagcatgtgctgcctatgatcccaattgggggcgcattcaaggacatgatcctggatggtgatggcaggactgtgtattaaggtacctttttctctaagcttggcttttctgctaacatatagcaatttttgaagaaaggaaagctagaaatacccaatatcttgctgaagcaagatattgggtatttcttttttttttattttgaatctttctattctgaattggcgccaagggtggccttgagtttctcgcccctttgccttaggattcggtaattctctttctcgaggggacggggaagggatataactcagcggtagagtgtcaccttgacgtggtggaagtcatcagttcgagcctgattatccctaaacccaatgtgagttttttctattttgacttactccccccgccacgatcgaacgggaatggataagaggcttgtgggattgacgtgatagggtagggttggctatactgctggtggcgaactccaggctaataatctgaagcgcatggatacaagttatccttggaaggaaagacaattccgaatccgctttgtctacgaataaggaagctataagtaatgcaactatgaatctcatggagagttcgatcctggctcaggatgaacgctggcggcatgcttaacacatgcaagtcgaacgggaagtggtgtttccagtggcgaacgggtgagtaacgcgtaagaacctgcccttgggaggggaacaacaactggaaacggttgctaataccccgtaggctgaggagcaaaaggagaaatccgcccaaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtggaaggcccacgggtcgtcaacttcttttctcggagaagaaacaatgacggtatctgaggaataagcatcggctaactctgtgccagcagccgcggtaagacagaggatgcaagcgttatccggaatgattgggcgtaaagcgtctgtaggtggcttttcaagtccgccgtcaaatcccagggctcaaccctggacaggcggtggaaactaccaagctggagtacggtaggggcagagggaatttccggtggagcggtgaaatgcattgagatcggaaagaacaccaacggcgaaagcactctgctgggccgacactgacactgagagacgaaagctaggggagcaaatgggattagagaccccagtagtcctagccgtaaacgatggatactaggtgctgtgcgactcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccctcgggaacgcggacacaggtggtgcatggctgtcgtcagctcgtgccgtaaggtgttgggttaagtctcgcaacgagcgcaaccctcgtgtttagttgccactatgagtttggaaccctgaacagaccgccggtgttaagccggaggaaggagaggatgaggccaagtcatcatgccccttatgccctgggcgacacacgtgctacaatgggcgggacaaagggtcgcgatctcgcgagggtgagctaactccaaaaacccgtcctcagttcggattgcaggctgcaactcgcctgcatgaagcaggaatcgctagtaatcgccggtcagccatacggcggtgaatccgttcccgggccttgtacacaccgcccgtcacactataggagctggccatgtttgaagtcattacccttaaccgtaaggagggggatgcctaaggctaggcttgcgactggagtgaagtcgtaacaaggtagccgtactggaaggtgcggctggatcacctccttttcagggagagctaatgcttatgcttattgggtattttggtttgacactgcttcacgcccaaaaagaaggcagctacgtctgagctaaacttggatatggaagtcttctttcgtttagggtgaagtaagaccaagct

SEQ ID NO: 108 - DNA, 2000 bp, synthetic construct
  source 1..2000: mol_type = other DNA
aagtcgaggtatttcgaattgtttgttgacacggacaaagtaagggaaaacccctgaaagaatttccatattgaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgattctttactgcgacagcatctagggctcctcgaataatgcgatatctcacaccgggtaaatccttaacccttcctcctcttactaatactacagaatgttcttgtaaattatggccaataccaggtatataagcagtgatttcaaatccagaggttaatcgtactctggcaactttacgtaaggcagagttgggttttttggggttgatagtggaaaagtcgacagataagtcacccttcctgtccctctacagaatcgtacatgagattttcacctaatacggctcctcgttcaattctttcgaagggatccttttcctcgttcgagagtcttcgccctttcttccactccgtcccgaagactaactaagaccaattgagtcacgttttcatgttctaattgaacactttccatttatgattaaaggagaagattgttcttttaccaaacatatgcggatcaaatcacgtcttataataagaagaaatctttctcggtatcaatccccttgcccctcattctttgagaatcagaaggatccttttcgagtttccatttcttcatttggaatctgggctcttctatcttcgacttattttttttttgctttattctttatttatttcatttcgatttttccctcttcctctatccctatcctctaggtacagcgtttgcatcaatagagaaccttttcctctgtatgaatctatattattccattccaatttcttcccgaaacttcccaagaaaaatcccgaattggatccaaaattgacgggttaatgtgagcttatccatgcggttaggcactcttcaaataggaatccattttctaactggctttcgtgctttggtgagtcgtccgagatcctttcgatgacctatgttgtgttgaagggatatctatatgatccgatcgattgcataagacccgcggtagcaatagaacggggaaagtatacagaaaagacagttcttttcaatttcgattatctatatattagttgtttctatttctagatatctatttctatatattagtattagttagtagtactattctattagttagcgatcccggctctgtgagttctttcttccgtgatgaactgtcggcaccagtcctccattttttctctgtggaccgaggagaaagggggctcagcaggaagaggattgtaccatgagagaagcacagaggtcaaccctcttcaaatatggaacatggattctgcaatgcaacgtagttgggtcctcatatcgatccgaatgaatcattctttctacagaggtcaatctttgcctattaggcaagaggatagcaagttcgaaattctgtctcggtaggacatggatttctattactatgaaattcataaatgaaatgaagtagttaatgggagggctaccattatcctttttcttgtatgtgttcctaagagaaggaatttgtccatttcatgtttcgaggtctcaaaaaagggcgtggaaacagataagaaactcttgaatggaaattgaaaagaaatgtagccccagttccttcggaaatggtaagatctttggcgcaagaagaaggggcgacccatatcatcttgacttggttctgcttcccctctttttttaagaataccgagtcgggttcttctcctaccagtatcgaatagaacatgctgaacaagatcttcttcatggaaacctgctcgatttagatcggg

SEQ ID NO: 109 - DNA, 2000 bp, synthetic construct
  source 1..2000: mol_type = other DNA
aagggtggccttgagtttctcgcccctttgccttaggattcggtaattctctttctcgaggggacggggaagggatataactcagcggtagagtgtcaccttgacgtggtggaagtcatcagttcgagcctgattatccctaaacccaatgtgagttttttctattttgacttactccccccgccacgatcgaacgggaatggataagaggcttgtgggattgacgtgatagggtagggttggctatactgctggtggcgaactccaggctaataatctgaagcgcatggatacaagttatccttggaaggaaagacaattccgaatccgctttgtctacgaataaggaagctataagtaatgcaactatgaatctcatggagagttcgatcctggctcaggatgaacgctggcggcatgcttaacacatgcaagtcgaacgggaagtggtgtttccagtggcgaacgggtgagtaacgcgtaagaacctgcccttgggaggggaacaacaactggaaacggttgctaataccccgtaggctgaggagcaaaaggagaaatccgcccaaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtggaaggcccacgggtcgtcaacttcttttctcggagaagaaacaatgacggtatctgaggaataagcatcggctaactctgtgccagcagccgcggtaagacagaggatgcaagcgttatccggaatgattgggcgtaaagcgtctgtaggtggcttttcaagtccgccgtcaaatcccagggctcaaccctggacaggcggtggaaactaccaagctggagtacggtaggggcagagggaatttccggtggagcggtgaaatgcattgagatcggaaagaacaccaacggcgaaagcactctgctgggccgacactgacactgagagacgaaagctaggggagcaaatgggattagagaccccagtagtcctagccgtaaacgatggatactaggtgctgtgcgactcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccctcgggaacgcggacacaggtggtgcatggctgtcgtcagctcgtgccgtaaggtgttgggttaagtctcgcaacgagcgcaaccctcgtgtttagttgccactatgagtttggaaccctgaacagaccgccggtgttaagccggaggaaggagaggatgaggccaagtcatcatgccccttatgccctgggcgacacacgtgctacaatgggcgggacaaagggtcgcgatctcgcgagggtgagctaactccaaaaacccgtcctcagttcggattgcaggctgcaactcgcctgcatgaagcaggaatcgctagtaatcgccggtcagccatacggcggtgaatccgttcccgggccttgtacacaccgcccgtcacactataggagctggccatgtttgaagtcattacccttaaccgtaaggagggggatgcctaaggctaggcttgcgactggagtgaagtcgtaacaaggtagccgtactggaaggtgcggctggatcacctccttttcagggagagctaatgcttatgcttattgggtattttggtttgacactgcttcacgcccaaaaagaaggcagctacgtctgagctaaacttggatatggaagtcttctttcgtttagggtgaagtaagaccaagct

SEQ ID NO: 110 - DNA, 30 bp, synthetic construct
  source 1..30: mol_type = other DNA
tcacattgggttgagctcaaagaaactagt

SEQ ID NO: 111 - DNA, 28 bp, synthetic construct
  source 1..28: mol_type = other DNA
cattgctatttgactcggacctgttctg

SEQ ID NO: 112 - DNA, 28 bp, synthetic construct
  source 1..28: mol_type = other DNA
tgttccgacctaggataataagctcatg

SEQ ID NO: 113 - DNA, 30 bp, synthetic construct
  source 1..30: mol_type = other DNA
atggtggtgcaattggaggataggttttat

SEQ ID NO: 114 - DNA, 18 bp, Oryza sativa
  source 1..18: mol_type = other DNA
caattgtcaaccggtcct

SEQ ID NO: 115 - DNA, 18 bp, Oryza sativa
  source 1..18: mol_type = other DNA
atggctcgaatccatagt

SEQ ID NO: 116 - DNA, 3162 bp, synthetic construct
  source 1..3162: mol_type = other DNA
atggccccctccgtgatggcgtcgtcggccaccaccgtcgctcccttccagggctcaagtccaccgccggcttgccgtcgcccgccgtccgaactccagcttcggcaacgtcagcatggcggcaggatcagatgcaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacacctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacatcggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcntgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttccaacggcggggggaaacaggccctggaaactgtgcagcgcctgctgccagtgctatgccaggctcacggcctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagcatgaatgaattaaccttgcaccctaacgtctacgcaacaggagcctccaagggtttgatacacgtattggaaagggtgtgggtaagggagcatactccaggtgatggaacaatatttgtcattagcggattcggaaattataatggaggtgtaaggttctttgaatccttcagggagcataaacgcaggggaggtgaattggttgctgtctttgcaggatcaacctcggctaggttgacttctaaacaagtagtccatgaaatgttggactgtggtgcagacgtacatattgtaaatcgcaaaaggttgcttcatgcaaaatgctatggtacgtccaaaatgagcggagacagcttgatagtcacaagcggtaactttacagggcctggtatgagccaaaatgtcgagatgagcgtgctcttggatgctgacacgactgcggaaatgggatttagctggggtacactcttggaaaatctgttggaccaaaggtggaattatcacagaccgacgctcggcgaccctgcgaaccctgcatggtcgttgctgtacgacgaacaagctgcggacatacgcctcgacgagacagaggaatga

SEQ ID NO: 117 - protein, 1053 aa, synthetic construct
MAPSVMASSATTVAPFQGSSPPPACRRPPSELQLRQRQHGGRIRCTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHTWRNALTGAPLNLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRXLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLPHAPALIKRTGSGSMNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 118 - DNA, 3162 bp, synthetic construct
  source 1..3162: mol_type = other DNA
atggctcccagcgtcatggcctccagcgcgacaactgtggccccgtttcagggttcgagcccgccacctgcttgccgccggccacctagcgagctgcaactccgccagcgccaacacggagggagaattagatgcaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacgggctgacccccgaacaggtggtggccattgccagccacgacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaagcactcgagacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatccatgaacgagttaacgctgcatccgaatgtgtatgcgaccggcgcgagtaaagggctgatacatgtgctggagcgcgtatgggttcgagaacacacgcctggggacggcacgatcttcgtgatatctggctttggcaactacaacggtggcgttcgcttcttcgagtcgtttcgtgaacacaagagacgtggcggggagctggtagcggtgttcgctggcagcacaagcgcgcgactgacaagcaaacaggttgttcacgagatgcttgattgcggggcggatgttcacatagtgaacagaaagcgactccttcacgctaagtgttacggcacaagcaagatgtcaggtgattccctaattgtaacctcaggcaatttcacaggtccgggaatgtcgcagaacgtggaaatgtctgtattgctagacgcagatacaaccgccgagatggggttctcgtggggcacgttgcttgagaacttgcttgatcagcgatggaactaccatcgccccaccttaggcgatccagcaaatccagcctggagcctgctttatgatgagcaggcagccgatattaggttggatgaaactgaagaatga

SEQ ID NO: 119 - protein, 1053 aa, synthetic construct
MAPSVMASSATTVAPFQGSSPPPACRRPPSELQLRQRQHGGRIRCTGRATMGKPIPNPLLGLDSTGGVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPQQVVAIASNGGGRPALESIVAQLSRPDPALAALTNDHLVALACLGGRPALDAVKKGLPHAPALIKRTGSGSMNELTLHPNVYATGASKGLIHVLERVWVREHTPGDGTIFVISGFGNYNGGVRFFESFREHKRRGGELVAVFAGSTSARLTSKQVVHEMLDCGADVHIVNRKRLLHAKCYGTSKMSGDSLIVTSGNFTGPGMSQNVEMSVLLDADTTAEMGFSWGTLLENLLDQRWNYHRPTLGDPANPAWSLLYDEQAADIRLDETEE

SEQ ID NO: 120 - DNA, 5910 bp, synthetic construct
  source 1..5910: mol_type = other DNA
agacatatatatctatctaaatttattgatatcaatatgaatgtggaaaatgctaaaataacttacattgtgaaatgaaggaagtaattttcttcacaaattttggcacttgcacttacggcttgtgaagcccgctcgatctgaagatttttcaaacgatgccattcaccacgtgccactttatatcaaaccttatttgtatccaagatttctggtactgggcattagcgatgtcaaacaaactcgacgtaaagttgagggacagagtgtgaacttattttattcccagcagagaacgcctccaaggtttatgggctttctggtccatcgcaacgacacggactttctacagggctgagattacgggcctaaatgggctcccccgtcttgtgctccgcgctgtttcaagttgggccaaaatggaactgggccgaatagtccggtgcggctaacggcatccacagccaagccaacggtgccgttctcccgctctcgcatttcaaaaatcgaaaataaaatcggcagggccgccgacgacgcaaccacccgttgggatccaatctactcctacgcaccgaacaactcatcgcacccaccgatatgtgggacccagctgtaacgtgggcccaccggcaagcgggacgtctgcatcggtcgcacctacaacgaggtggccaggtcacctactgctttttcttggagcgacgcgtgcactcgctaaccgcacacgaaggaagcctctggacccacctgtcagtgaaactaacgagaggcgggtatggacttcaaccgagtgcaaacgaaatggcctccttgcggtggatattatcagaaagctccctaacttattaaatattctcgatatgatccaaccacccgacctttgcaggcacgatgtgagatgcgagtgaattttgaagacgtcaaagggggtaaaacgaaaaactcccccatcccttcgcttccgctcctgagaggtccaggctgtctggccctataaaacgggcggagtgcggggatcgcaaaccctaagccgagacaacgcagagaaaggcgtcttcgtactcgcctctctccgcgcctccgcgcttttcctcctctcccctctctcccttctccgccgccgtcgcagcatcaacccaatccgccgccaagcttcatccaggtacggatccgcgtcccatctccctcaccccccgtgttcttcgtgcctgcttctgggtcagatctgggtggattcgcggttgttggatgtggggggctgtgtttatttgtcggtggatctggttgtctggatctgcgttttctctgtcgtagttagcggatctgatgaaatgtttagtgttcgtgtatactggtatggtggatctggtcctaggatgcgtggaatggatatatgtaggcgaattggaggatttattttgtgaattttgctgaaatgatagttctaaacactggatctgacctcgggatgctgttaaatgtggaaatcatggtcgatgctgtcatgaacatggtgttcttatggtagatctgagcaatgtatgtttcaaaattgtttgtcacatggaaatgctatggttctagatgcaatagaatgatacatgccgagatcccctctagttgatatgatagatcatgatgttttacagctatgtcatatgaatatgttcatttgttaccgatgtatttggatctacttaacatttccaaagcacgccgcgttctaattctagatctggtagtcatgtttgtacacgtcatccacctaatacaaatacatatgtctagtgtttggtgacactgcccgtcagatctgttttttccagatctgtggaacaaatactccatgcatgtatggtagttttgaaacgatcttgtatcttccattgttgtagtaacaactaaataaagtacaattgttcaattattgggaatcgtattttctgtagtgccgatgtacagcatattcatagatgtctatttaggaactcaaattttaaattgaggactagttatttattatgggtcagtcttttgaattgtgttatcttgctgtactgaaataataatgtaccactaaggcgctaacatgtattttgtcctttcaggctgactcgacggatcccggggggcaagagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaagatggccccctccgtgatggcgtcgtcggccaccaccgtcgctcccttccagggctcaagtccaccgccggcttgccgtcgcccgccgtccgaactccagcttcggcaacgtcagcatggcggcaggatcagatgcaccggccgcgccaccatgggaaaacctattcctaatcctctgctgggcctggattctaccggaggcgtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacgaggcgctggagccagagccctggaagccctgctgacagtggccggagaactgagaggacctcctctgcagctggatacaggacagctgctgaagattgccaaaaggggcggagtgaccgcggtggaagccgtgcacacctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagcaacggcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacatcggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcntgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctaacaacggaggaaaacaagcactcgagacagtgcagcggctgctgcctgtgctgtgtcaggctcacggcttgactccagaacaggtggtggctattgcttccaacggcggggggaaacaggccctggaaactgtgcagcgcctgctgccagtgctatgccaggctcacggcctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagaccggacccagctctcgctgcattgactaacgaccatctcgtcgctttggcttgcctcggtggaaggccagctcttgacgctgtaaagaaaggtttgccacatgctccagctcttattaaaaggactggtagcggtagcatgaatgaattaaccttgcaccctaacgtctacgcaacaggagcctccaagggtttgatacacgtattggaaagggtgtgggtaagggagcatactccaggtgatggaacaatatttgtcattagcggattcggaaattataatggaggtgtaaggttctttgaatccttcagggagcataaacgcaggggaggtgaattggttgctgtctttgcaggatcaacctcggctaggttgacttctaaacaagtagtccatgaaatgttggactgtggtgcagacgtacatattgtaaatcgcaaaaggttgcttcatgcaaaatgctatggtacgtccaaaatgagcggagacagcttgatagtcacaagcggtaactttacagggcctggtatgagccaaaatgtcgagatgagcgtgctcttggatgctgacacgactgcggaaatgggatttagctggggtacactcttggaaaatctgttggaccaaaggtggaattatcacagaccgacgctcggcgaccctgcgaaccctgcatggtcgttgctgtacgacgaacaagctgcggacatacgcctcgacgagacagaggaatgagaattcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 121 - DNA, 5807 bp, synthetic construct
  source 1..5807: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggctcccagcgtcatggcctccagcgcgacaactgtggccccgtttcagggttcgagcccgccacctgcttgccgccggccacctagcgagctgcaactccgccagcgccaacacggagggagaattagatgcaccggccgcgccaccatgggaaaaccgatcccaaacccgcttttggggctcgacagcacaggaggggttgatctcaggaccctcggttacagccaacagcaacaagaaaaaattaagcctaaagtgaggtccacggtagctcaacatcacgaggctttggtagggcatggcttcacgcacgctcatatcgtcgctctcagccaacatcccgctgcgcttgggacggtcgctgttaagtaccaagacatgatcgctgcgcttccggaagctacgcatgaggcgatcgtcggtgtagggaagcggggagcaggtgcgagggcgctcgaggcgctcttgaccgtcgcgggtgagcttaggggtccgccactccaattggacaccggacaacttttgaaaatcgctaagagaggaggcgtcacagccgtcgaggcggtgcacgcctggagaaatgccctgacaggagcccctctgaacctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacgggctgacccccgaacaggtggtggccattgccagccacgacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggcctcactcccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaagcactcgagacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggcctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaggatccggatccatgaacgagttaacgctgcatccgaatgtgtatgcgaccggcgcgagtaaagggctgatacatgtgctggagcgcgtatgggttcgagaacacacgcctggggacggcacgatcttcgtgatatctggctttggcaactacaacggtggcgttcgcttcttcgagtcgtttcgtgaacacaagagacgtggcggggagctggtagcggtgttcgctggcagcacaagcgcgcgactgacaagcaaacaggttgttcacgagatgcttgattgcggggcggatgttcacatagtgaacagaaagcgactccttcacgctaagtgttacggcacaagcaagatgtcaggtgattccctaattgtaacctcaggcaatttcacaggtccgggaatgtcgcagaacgtggaaatgtctgtattgctagacgcagatacaaccgccgagatggggttctcgtggggcacgttgcttgagaacttgcttgatcagcgatggaactaccatcgccccaccttaggcgatccagcaaatccagcctggagcctgctttatgatgagcaggcagccgatattaggttggatgaaactgaagaatgagaattcggtaccgagtttgaatcaaatcttcacttgtttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcggga

SEQ ID NO: 122 - DNA, 142 bp, Oryza sativa
  source 1..142: mol_type = other DNA
cctccgggctttctttccacactattatggatagtcaaataatgggaaaaattggattcaattgtcaaccggtcctatcgaaaataggattgactatggattcgagccatagcacatggtttcataaaatctgtacgatttt

SEQ ID NO: 123 - DNA, 21 bp, Oryza sativa
  source 1..21: mol_type = other DNA
ctccgggctttctttccacac

SEQ ID NO: 124 - DNA, 30 bp, Oryza sativa
  source 1..30: mol_type = other DNA
aatcgtacagattttatgaaaccatgtgct

SEQ ID NO: 125 - DNA, 721 bp, synthetic construct
  source 1..721: mol_type = other DNA
gaaaaggtcaagtctaagcgacatatggcacgaaaaggaaatccaatttcggtaatccgaagcccaccttaccttaatccattttcttgaggatcttgcatttgttcgttctctgccgacctattgtttccatcactcctgttgcttcttcctttctctatccaacccgagctctatcgcccatggctgaactcttctctgcagcctcatcgccaacaaaaggtccacatccttcaatcggcatgtactatggaatggctttaatgtttctatatccggaagtagggaatctcgtgcttgcataatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactat

SEQ ID NO: 126 - protein, 352 aa, Oryza rufipogon
MTRDRMRQSIKGRALCRAFSSRSAGGVIFKVVLKSLQERKKRISSEISSTIYFYMLCIPYLIFFLKISILVFLVFYRIVLPIVQMFSLLTCCSFLILILLFCLILTCLILFRLKKKEDFASPPESIISIFSSLFAILMAVLASGPSVAYAMENPGPSEAGPEGSAAPPSSSFQESTGEMDALMASTTPSAPRTPSGGEPSVNQPLPGEQAMPPALPVMQEAANRAPPYAPYPYPVDEIIGGDSVQSIQRRLLGTNWNPSAHDIKMSRIQAEDLFELKVEIIRKMAGLHPSGDWMGWGARALDNPRTATGEEDLARLHQMLDDLQSRNEQSATFWRLVERVRLRADEDQNSAS

SEQ ID NO: 127 - protein, 352 aa, Oryza rufipogon
MTRDRMRQSIKGRALCRAFSSRSAGGVIFKVVLKSLQERKKRISSEISSTIYFYMLCIPYLIFFLKISILVFLVFYRIVLPIVQMFSLLTCCSFLILILLFCLILTCLILFRLKKKEDFASPPESIISIFSSIFAILMAVLASGPSVAYAMENPGPSEAGPEGSAAPPSSSFQESTGEMDALMASTTPSAPRTPSGGEPSVNQPLPGEQAMPPALPVMQEAANRSPPYAPYPYPVDEIIGGDSVQSIQRRLLGTNWNPSAHDMQMSRIQAEDLFELKVEIIRKMAGLHPSGDWMGWGARALDNPRTATGEEDLARLHQMLDDLQSRNEQSATFWRLVERVRLRADEDQNSAS

SEQ ID NO: 128 - protein, 352 aa, Oryza sativa; identical to SEQ ID NO: 127

SEQ ID NO: 129 - protein, 59 aa, Chlamydomonas reinhardtii
MALRRAVASFLPKLAGAAETLPAASHAASSFSQLICTPLDVVERQQQPSGFRSFASDAV